(19) United States
(12) Patent Application Publication
(10) Pub. No.: US 2026/0151375 A1
(43) Pub. Date: Jun. 4, 2026

(54) CDK2 INHIBITORS AND METHODS OF USING THE SAME

(71) Applicant: Cedilla Therapeutics, Inc., Foxboro, MA (US)

(72) Inventors: Louise Clare KIRMAN, Foxboro, MA (US); Carl Eric SCHWARTZ, Foxboro, MA (US); Thomas P. BLAISDELL, Foxboro, MA (US)

(21) Appl. No.: 18/995,329

(22) PCT Filed: Jul. 28, 2023
(86) PCT No.: PCT/EP2023/071252

§ 371 Date: Jan. 16, 2025

Related U.S. Application Data
(60) Provisional application No. 63/393,714, filed on Jul. 29, 2022.

(51) Int. Cl. A61K 31/422; A61K 31/4196; A61K 31/4245; A61K 31/427; A61K 31/428; A61K 31/438; A61K 31/4439; A61K 31/454; A61K 31/4725; A61K 31/497; A61K 31/498; A61K 31/501; A61K 31/506; A61K 31/519; A61P 35/00; C07D 487/10; C07D 519/00

(52) U.S. Cl. CPC A61K 31/422; A61K 31/4196; A61K 31/4245; A61K 31/427; A61K 31/428; A61K 31/438; A61K 31/4439; A61K 31/454; A61K 31/4725; A61K 31/497; A61K 31/498; A61K 31/501; A61K 31/506; A61K 31/519; A61P 35/00; C07D 487/10; C07D 519/00

(57) ABSTRACT

The present disclosure provides compounds, compositions thereof, and methods of using the same for the inhibition of CDK2, and the treatment of CDK2 related diseases and disorders.

### CROSS REFERENCE TO RELATED APPLICATIONS

This application claims the benefit of priority to U.S. Provisional Application No. 63/393,714, filed Jul. 29, 2022, the entire contents of which is herein incorporated by reference.

### FIELD

The present disclosure relates generally to Cyclin-dependent kinase 2 (CDK2) inhibiting chemical compounds and uses thereof in the inhibition of the activity of CDK2. The disclosure also provides pharmaceutically acceptable compositions comprising compounds disclosed herein and methods of using said compounds and compositions in the treatment of various disorders related to CDK2 activity.

### BACKGROUND

Cell cycle dysregulation, including uncontrolled cell growth, impaired cell differentiation and abnormal apoptosis have been shown to be caused by over activity of Cyclin-dependent kinases (CDKs). CDKs are important serine/threonine protein kinases that become active when combined with a specific cyclin partner. There are various subtypes of CDKs, each having a different role during the cell cycle, with varying levels of activity during each of the phases. CDK1, CDK2, CDK4 and CDK6 have been found to be specifically important subtypes, where over activity of one or more of these subtypes may lead to dysregulation of the cell cycle and the development of a variety of cancers. The S phase of the cell cycle is responsible for DNA replication and is the phase where aberrant DNA replication may occur. The CDK2/cyclin E complex is required for the cell cycle transition from the G1 phase to the S phase and the CDK2/cyclin A complex is required for the cell cycle transition from the S phase to the G2 phase. Therefore, selective inhibition of the CDK2/cyclin E and/or CDK2/cyclin A complexes can prevent aberrant DNA replication and can be used to treat certain cancers.

Accordingly, there is a need for the development of compounds capable of inhibiting the activity of CDK2/cyclin complexes, and pharmaceutical compositions thereof, for the prevention, and treatment of CDK2 related diseases or disorders.

### SUMMARY

The present disclosure is based at least in part on the identification of compounds that bind and inhibit Cyclin-dependent kinase 2 (CDK2) and/or CDK2/cyclin complexes and methods of using the same to treat diseases associated with CDK2 activity. Disclosed herein is a compound according to Formula I or a pharmaceutically acceptable salt thereof:

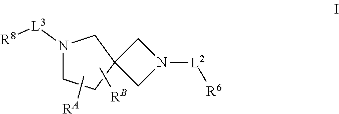

wherein each variable is as defined and described herein.

Compounds of the present disclosure, and pharmaceutically acceptable compositions thereof, are useful for treating a variety of diseases, disorders or conditions, associated with CDK2 activity. Such diseases, disorders, or conditions include those described herein.

### DETAILED DESCRIPTION OF CERTAIN EMBODIMENTS

### 1. General Description of Compounds of the Disclosure

The present disclosure provides compounds capable of inhibiting Cyclin-dependent kinase 2 (CDK2) and/or CDK2/cyclin complexes.

In certain embodiments, the present disclosure provides inhibitors of CDK2 activity. In some embodiments, the inhibitors of CDK2 include compounds of Formula I:

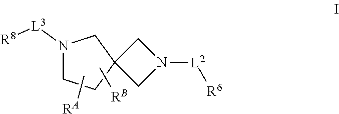

or a pharmaceutically acceptable salt thereof, wherein:
is N or CR^{B};
R^{A} is

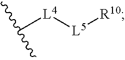

each R^{B} is independently a hydrogen, an optionally substituted C₁₋₆ aliphatic group, or a halogen;
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —C(R)₂—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy¹ group, or Cy¹;
L³ is a covalent bond or a saturated or unsaturated, straight or branched, optionally substituted bivalent C₁₋₄ hydrocarbon chain, wherein 0-4 methylene units of L³ are independently replaced by —O—, —NR—, —S—, —OC(O)—, —C(O)O—, —C(O)—, —S(O)—, —S(O)₂—, —C(S)—, —C(R)₂—, —NRS(O)₂—, —S(O)₂NR—, —NRC(O)—, —C(O)NR—, —OC(O)NR—, —NRC(O)O—, —NRC(O)NR—, or -Cy²-;
L⁴ is optionally substituted phenylene, an optionally substituted bivalent 5-6 membered monocyclic heteroarylene ring (having 1-4 heteroatoms independently selected from nitrogen, oxygen, and sulfur), or an optionally substituted bivalent 8-10 membered bicyclic heteroarylene ring (having 1-5 heteroatoms independently selected from nitrogen, oxygen, and sulfur);
L⁸ is a covalent bond or a saturated or unsaturated, straight or branched, optionally substituted bivalent C₁₋₄ hydrocarbon chain, wherein 0-2 methylene units of L⁵ are independently replaced by —O—, —NR—, —S—, —C(R)₂—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy²-, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted saturated or unsaturated C₁₋₆ aliphatic group, an optionally substituted C₁₋₆ aliphatic-Cy¹ group, or Cy¹;
R¹⁰ is hydrogen or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each Cy¹ is independently an optionally substituted and cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur);
each -Cy²- is independently an optionally substituted and bivalent cyclic group selected from a 3-8 membered saturated or partially unsaturated monocyclic carbocyclene, phenylene, a 3-8 membered saturated or partially unsaturated monocyclic heterocyclene ring (having 1-2 heteroatoms independently selected from nitrogen, oxygen, and sulfur), and a 5-6 membered monocyclic heteroarylene ring (having 1-4 heteroatoms independently selected from nitrogen, oxygen, and sulfur); and
each R is independently hydrogen, halogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), an optionally substituted 5-6 membered heteroaryl ring (having 1-4 heteroatoms independently selected from nitrogen, oxygen, and sulfur), two R groups on the same nitrogen atom or carbon atom are taken together with the nitrogen atom or carbon atom to form an optionally substituted 3-7 membered saturated, partially unsaturated, or heteroaryl ring (having 0-3 heteroatoms independently selected from nitrogen, oxygen, and sulfur), or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur).

Overexpression of CDK2 is associated with abnormal regulation of the cell-cycle. The cyclin E/CDK2 complex plays an important role in regulation of the G1/S transition, histone biosynthesis and centrosome duplication. Progressive phosphorylation of retinoblastoma (Rb) by cyclin D/Cdk4/6 and cyclin E/Cdk2 releases the G1 transcription factor, E2F, and promotes S-phase entry. Activation of cyclin A/CDK2 during early S-phase promotes phosphorylation of endogenous substrates that permit DNA replication and inactivation of E2F, for S-phase completion. (Asghar et al., Nat. Rev. Drug. Discov. 2015; 14(2): 130-146).

Cyclin E, the regulatory cyclin for CDK2, is frequently overexpressed in cancer. Cyclin E amplification or overexpression has long been associated with poor outcomes in breast cancer. (Keyomarsi et al., Cyclin E and survival in patients with breast cancer. N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Mol. Cancer Ther. (2012) 11:1488-99; Herrera-Abreu et al., Cancer Res. (2016) 76: 2301-2313). Cyclin E amplification also reportedly contributes to trastuzumab resistance in HER2+ breast cancer. (Scaltriti et al., Proc Natl Acad Sci. (2011) 108: 3761-6). Cyclin E overexpression has also been reported to play a role in basal-like and triple negative breast cancer (TNBC), as well as inflammatory breast cancer. (Elsawaf & Sinn, Breast Care (2011) 6:273-278; Alexander et al., Oncotarget (2017) 8: 14897-14911.)

Amplification or overexpression of cyclin E1 (CCNE1) is also associated with poor outcomes in ovarian, gastric, endometrial and other cancers. (Nakayama et al., Gene amplification CCNE1 is related to poor survival and potential therapeutic target in ovarian cancer, Cancer (2010) 116: 2621-34; Etemadmoghadam et al., Clin Cancer Res (2013) 19: 5960-71; Au-Yeung et al., Clin. Cancer Res. (2017) 23:1862-1874; Ayhan et al., Modern Pathology (2017) 30: 297-303; Ooi et al., Hum Pathol. (2017) 61: 58-67; Noske et al., Oncotarget (2017) 8: 14794-14805).

There remains a need in the art for CDK inhibitors, especially selective CDK2 inhibitors, which may be useful for the treatment of cancer or other proliferative diseases or conditions. In particular, CDK2 inhibitors may be useful in treating CCNE1 or CCNE2 amplified tumors.

### 2. Compounds and Definitions

Compounds of this present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1 to 6 aliphatic carbon atoms. In some embodiments, aliphatic groups contain 1 to 5 aliphatic carbon atoms. In other embodiments, aliphatic groups contain 1 to 4 aliphatic carbon atoms. In still other embodiments, aliphatic groups contain 1 to 3 aliphatic carbon atoms, and in yet other embodiments, aliphatic groups contain 1 to 2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Bridged bicyclic groups and spirocyclic groups are within the scope of “bicyclic” groups. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

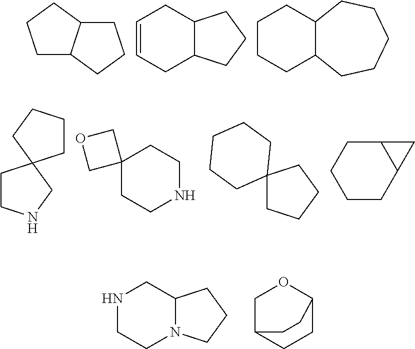

Exemplary bridged bicyclics include:

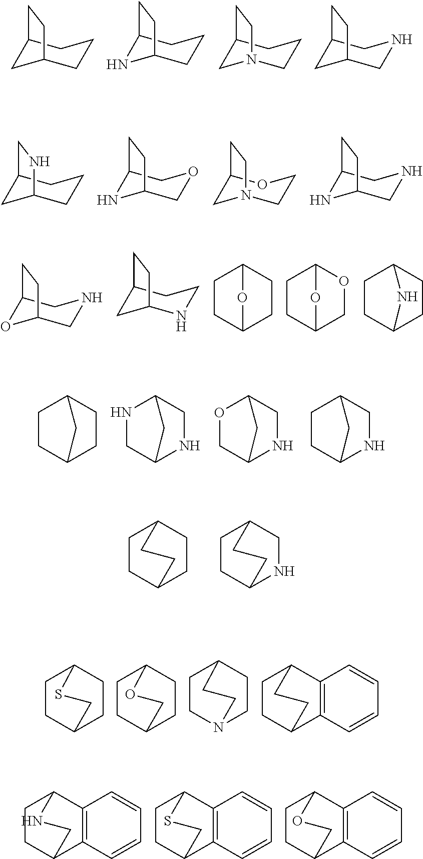

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon including, any oxidized form of nitrogen, sulfur, phosphorus, or silicon; the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7 to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring having 0 to 3 heteroatoms selected from oxygen, sulfur and nitrogen.

A heterocyclic ring can be attached to a provided compound at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic or bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₆R^{◯}; —(CH₂)₀₋₆OR^{◯}; —O(CH₂)₀₋₆R^{◯}, —O—(CH₂)₀₋₆C(O)OR^{◯}; —(CH₂)O—CH(OR^{◯})₂; —(CH₂)₀₋₆SR^{◯}; —(CH₂)₀₋₆Ph, which Ph may be substituted with R^{◯}; —(CH₂)₀₋₄₆O(CH₂)₀₋₁Ph which Ph may be substituted with R^{◯}; —CH═CHPh, which Ph may be substituted with R^{◯}; —(CH₂)₀₋₆O(CH₂)₀₋₁-pyridyl which pyridyl may be substituted with R^{◯}; —NO₂; —CN; —N₃; —(CH₂)₀₋₆N(R^{◯})₂; —(CH₂)₀₋₆N(R^{◯})C(O)R^{◯}; —N(R^{◯})C(S)R^{◯}; —(CH₂)_{0- 6}N(R^{◯})C(O)NR^{◯}₂; —N(R^{◯})C(S)NR^{◯}₂; —(CH₂)₀₋₆N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₆C(O)R^{◯}; —C(S)R^{◯}; —(CH₂)₀₋₆C(O)OR^{◯}; —(CH₂)₀₋₆—C(O)SR^{◯}; —(CH₂)₀₋₆C(O)OSiR^{◯}₃; —(CH₂)₀₋₆OC(O)R^{◯}; —OC(O)(CH₂)₀₋₆SR^{◯}, —(CH₂)₀₋₆SC(O)R^{◯}; —(CH₂)₀₋₆C(O)NR^{◯}₂; —C(S)NR^{◯}₂; —C(S)SR^{◯}; —SC(S)SR^{◯}, —(CH₂)₀₋₆OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₆SSR^{◯}; —(CH₂)₀₋₆S(O)₂R^{◯}; —(CH₂)₀₋₆S(O)₂OR^{◯}; —(CH₂)₀₋₆OS(O)₂R^{◯}; —S(O)₂NR^{◯}₂; —(CH₂)₀₋₆S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂, —P(O)₂R^{◯}; —P(O)R^{◯}₂, —P(O)(OR^{◯})₂; —OP(O)(R^{◯})OR^{◯}; —OP(O)R^{◯}₂; —OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, saturated or unsaturated C₁₋₆ aliphatic, —CH₂Ph, —O(CH₂)₀₋₁Ph, —CH₂-(5- to 6-membered heteroaryl ring), a 3- to 6-membered saturated, partially unsaturated, or aryl ring (having 0 to 4 heteroatoms independently selected from nitrogen, oxygen, and sulfur), an 8 to 12-membered saturated, partially unsaturated, or aryl bicyclic ring (having 0 to 4 heteroatoms independently selected from nitrogen, oxygen, and sulfur), or, notwithstanding the definition above, two independent occurrences of R^{◯}, taken together with their intervening atom(s), form a 3- to 12-membered saturated, partially unsaturated, or aryl mono- or bicyclic ring (having 0 to 4 heteroatoms independently selected from nitrogen, oxygen, and sulfur), which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted, is substituted with one or more methyl, —CO₂H, oxo, or hydroxyl, or where preceded by “halo” is substituted with one or more halogens, and is independently selected from C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur), and said “haloR^{●}” may be further substituted with hydroxyl, amino, or C₁₋₄ alkoxy. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur). Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur).

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5 to 6-membered saturated, partially unsaturated, or aryl ring (having 0 to 4 heteroatoms independently selected from nitrogen, oxygen, and sulfur), or, notwithstanding the definition above, two independent occurrences of R^{†}, taken together with their intervening atom(s) form an unsubstituted 3 to 12-membered saturated, partially unsaturated, or aryl mono- or bicyclic ring (having 0 to 4 heteroatoms independently selected from nitrogen, oxygen, and sulfur).

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

As used herein, the term “provided compound” or “compound of the present disclosure” refers to any genus, subgenus, and/or species set forth herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

As used herein, the term “inhibitor” is defined as a compound that binds to and/or inhibits CDK2 with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM, when measured in an appropriate assay.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily or degratorily active metabolite or residue thereof.

As used herein, the term “inhibitorily active metabolite or residue thereof” means that a metabolite or residue thereof is also an inhibitor of a CDK2 protein, or a mutant thereof. 3. Description of Exemplary Embodiments:

In certain embodiments, the present disclosure provides inhibitors of CDK2 activity. In some embodiments, the inhibitors of CDK2 include compounds of Formula I:

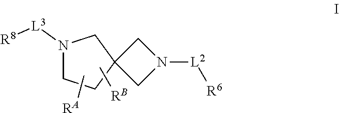

or a pharmaceutically acceptable salt thereof, wherein:
X is N or CR;
R^{A} is

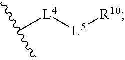

each R^{B} is independently a hydrogen, an optionally substituted C₁₋₆ aliphatic group, or a halogen;
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —C(R)₂—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy¹ group, or Cy¹;
L³ is a covalent bond or a saturated or unsaturated, straight or branched, optionally substituted bivalent C₁₋₄ hydrocarbon chain, wherein 0-4 methylene units of L³ are independently replaced by —O—, —NR—, —S—, —OC(O)—, —C(O)O—, —C(O)—, —S(O)—, —S(O)₂—, —C(S)—, —C(R)₂—, —NRS(O)₂—, —S(O)₂NR—, —NRC(O)—, —C(O)NR—, —OC(O)NR—, —NRC(O)O—, —NRC(O)NR—, or -Cy²-;
L⁴ is optionally substituted phenylene, an optionally substituted bivalent 5-6 membered monocyclic heteroarylene ring (having 1-4 heteroatoms independently selected from nitrogen, oxygen, and sulfur), or an optionally substituted bivalent 8-10 membered bicyclic heteroarylene ring (having 1-5 heteroatoms independently selected from nitrogen, oxygen, and sulfur);
L⁵ is a covalent bond or a saturated or unsaturated, straight or branched, optionally substituted bivalent C₁₋₄ hydrocarbon chain, wherein 0-2 methylene units of L⁵ are independently replaced by —O—, —NR—, —S—, —C(R)₂—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy²-, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted saturated or unsaturated C₁₋₆ aliphatic group, an optionally substituted C₁₋₆ aliphatic-Cy¹ group, or Cy¹;
R¹⁰ is hydrogen or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each Cy¹ is independently an optionally substituted and cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur);
each -Cy²- is independently an optionally substituted and bivalent cyclic group selected from a 3-8 membered saturated or partially unsaturated monocyclic carbocyclene, phenylene, a 3-8 membered saturated or partially unsaturated monocyclic heterocyclene ring (having 1-2 heteroatoms independently selected from nitrogen, oxygen, and sulfur), and a 5-6 membered monocyclic heteroarylene ring (having 1-4 heteroatoms independently selected from nitrogen, oxygen, and sulfur); and
each R is independently hydrogen, halogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), an optionally substituted 5-6 membered heteroaryl ring (having 1-4 heteroatoms independently selected from nitrogen, oxygen, and sulfur), two R groups on the same nitrogen atom or carbon atom are taken together with the nitrogen atom or carbon atom to form an optionally substituted 3-7 membered saturated, partially unsaturated, or heteroaryl ring (having 0-3 heteroatoms independently selected from nitrogen, oxygen, and sulfur), or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur).

As defined generally above, X is N or CR^{B}. In some embodiments, X is N. In some embodiments, X is CR^{B}. In some embodiments, X is CH.

As defined generally above, R^{A} is

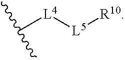

In some embodiments, R^{A} is selected from Table 1, below. In some embodiments, R^{A} is selected from those depicted in the compounds of Table 8, below.

TABLE 1
Exemplary R^{A} substituents

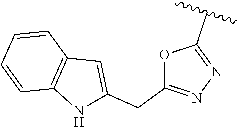 [SMILES: *c1nnc(Cc2cc3ccccc3[nH]2)o1]

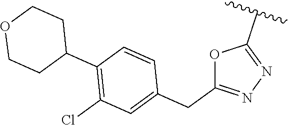 [SMILES: *c1nnc(Cc2ccc(C3CCOCC3)c(Cl)c2)o1]

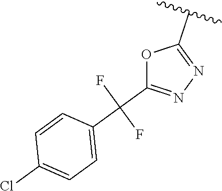 [SMILES: *c1nnc(C(F)(F)c2ccc(Cl)cc2)o1]

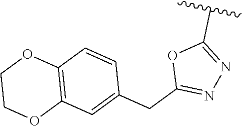 [SMILES: *c1nnc(Cc2ccc3c(c2)OCCO3)o1]

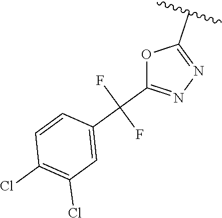 [SMILES: *c1nnc(C(F)(F)c2ccc(Cl)c(Cl)c2)o1]

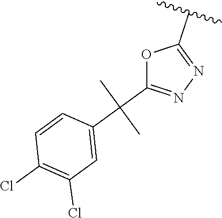 [SMILES: *c1nnc(C(C)(C)c2ccc(Cl)c(Cl)c2)o1]

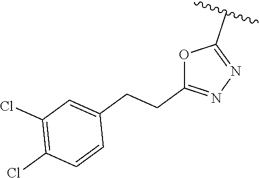 [SMILES: *c1nnc(CCc2ccc(Cl)c(Cl)c2)o1]

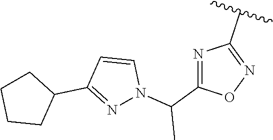 [SMILES: *c1noc(C(C)n2ccc(C3CCCC3)n2)n1]

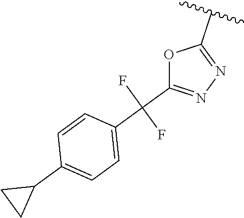 [SMILES: *c1nnc(C(F)(F)c2ccc(C3CC3)cc2)o1]

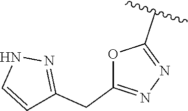 [SMILES: *c1nnc(Cc2cc[nH]n2)o1]

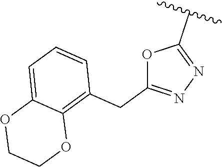 [SMILES: *c1nnc(Cc2cccc3c2OCCO3)o1]

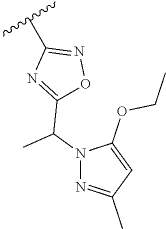 [SMILES: *c1noc(C(C)n2nc(C)cc2OCC)n1]

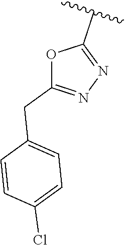 [SMILES: *c1nnc(Cc2ccc(Cl)cc2)o1]

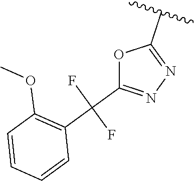 [SMILES: *c1nnc(C(F)(F)c2ccccc2OC)o1]

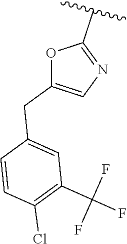 [SMILES: *c1ncc(Cc2ccc(Cl)c(C(F)(F)F)c2)o1]

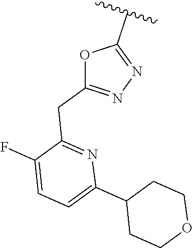 [SMILES: *c1nnc(Cc2nc(C3CCOCC3)ccc2F)o1]

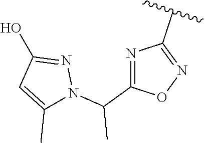 [SMILES: *c1noc(C(C)n2nc(O)cc2C)n1]

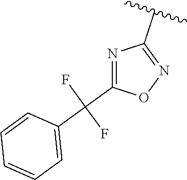 [SMILES: *c1noc(C(F)(F)c2ccccc2)n1]

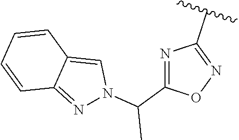 [SMILES: *c1noc(C(C)n2cc3ccccc3n2)n1]

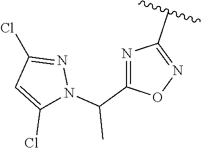 [SMILES: *c1noc(C(C)n2nc(Cl)cc2Cl)n1]

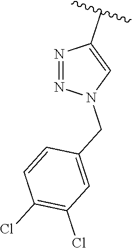 [SMILES: *c1cn(Cc2ccc(Cl)c(Cl)c2)nn1]

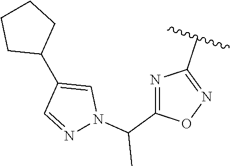 [SMILES: *c1noc(C(C)n2cc(C3CCCC3)cn2)n1]

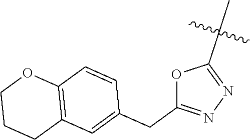 [SMILES: *c1nnc(Cc2ccc3c(c2)CCCO3)o1]

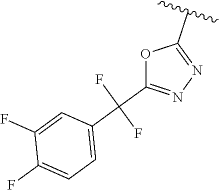 [SMILES: *c1nnc(C(F)(F)c2ccc(F)c(F)c2)o1]

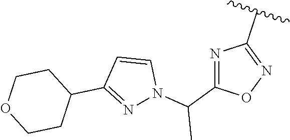 [SMILES: *c1noc(C(C)n2ccc(C3CCOCC3)n2)n1]

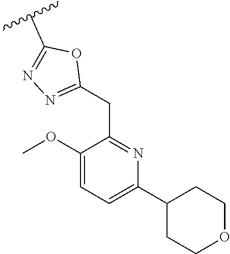 [SMILES: *c1nnc(Cc2nc(C3CCOCC3)ccc2OC)o1]

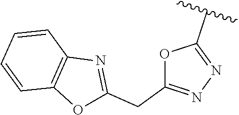 [SMILES: *c1nnc(Cc2nc3ccccc3o2)o1]

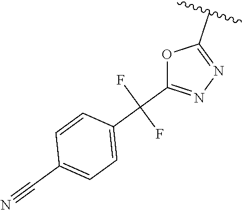 [SMILES: *c1nnc(C(F)(F)c2ccc(C#N)cc2)o1]

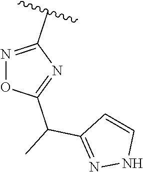 [SMILES: *c1noc(C(C)c2cc[nH]n2)n1]

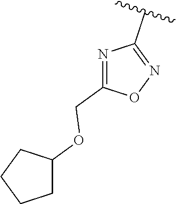 [SMILES: *c1noc(COC2CCCC2)n1]

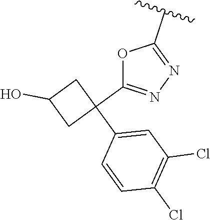 [SMILES: *c1nnc(C2(c3ccc(Cl)c(Cl)c3)CC(O)C2)o1]

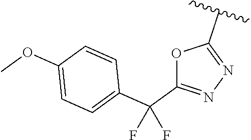 [SMILES: *c1nnc(C(F)(F)c2ccc(OC)cc2)o1]

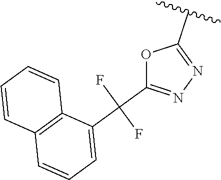 [SMILES: *c1nnc(C(F)(F)c2cccc3ccccc23)o1]

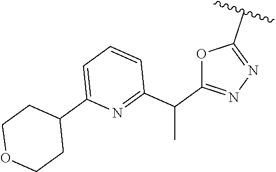 [SMILES: *c1nnc(C(C)c2cccc(C3CCOCC3)n2)o1]

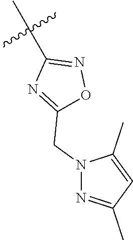 [SMILES: *c1noc(Cn2nc(C)cc2C)n1]

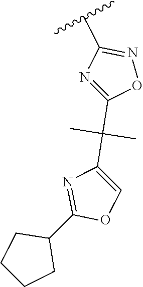 [SMILES: *c1noc(C(C)(C)c2coc(C3CCCC3)n2)n1]

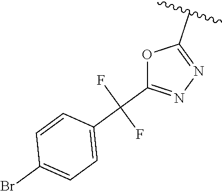 [SMILES: *c1nnc(C(F)(F)c2ccc(Br)cc2)o1]

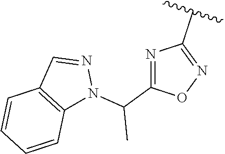 [SMILES: *c1noc(C(C)n2ncc3ccccc32)n1]

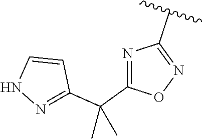 [SMILES: *c1noc(C(C)(C)c2cc[nH]n2)n1]

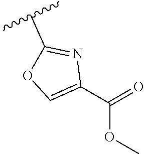 [SMILES: *c1nc(C(=O)OC)co1]

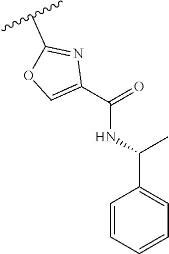 [SMILES: *c1nc(C(=O)N[C@H](C)c2ccccc2)co1]

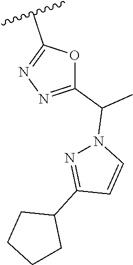 [SMILES: *c1nnc(C(C)n2ccc(C3CCCC3)n2)o1]

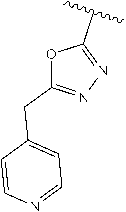 [SMILES: *c1nnc(Cc2ccncc2)o1]

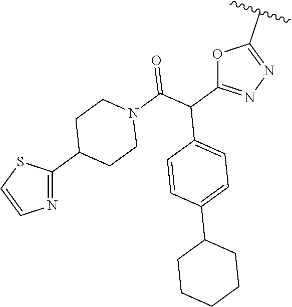 [SMILES: *c1nnc(C(C(=O)N2CCC(c3nccs3)CC2)c2ccc(C3CCCCC3)cc2)o1]

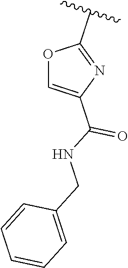 [SMILES: *c1nc(C(=O)NCc2ccccc2)co1]

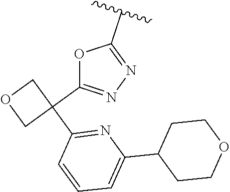 [SMILES: *c1nnc(C2(c3cccc(C4CCOCC4)n3)COC2)o1]

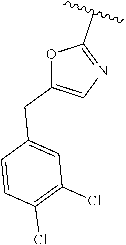 [SMILES: *c1ncc(Cc2ccc(Cl)c(Cl)c2)o1]

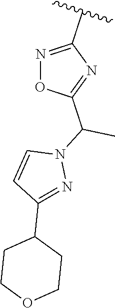 [SMILES: *c1noc(C(C)n2ccc(C3CCOCC3)n2)n1]

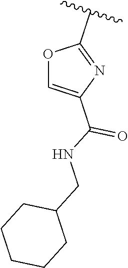 [SMILES: *c1nc(C(=O)NCC2CCCCC2)co1]

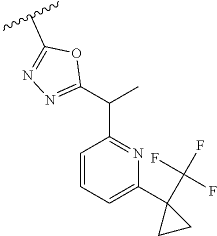 [SMILES: *c1nnc(C(C)c2cccc(C3(C(F)(F)F)CC3)n2)o1]

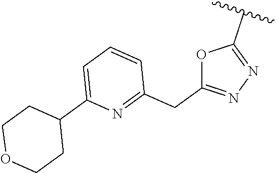 [SMILES: *c1nnc(Cc2cccc(C3CCOCC3)n2)o1]

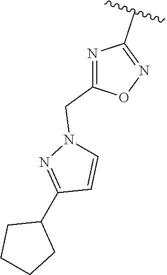 [SMILES: *c1noc(Cn2ccc(C3CCCC3)n2)n1]

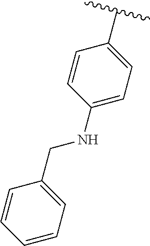 [SMILES: *c1ccc(NCc2ccccc2)cc1]

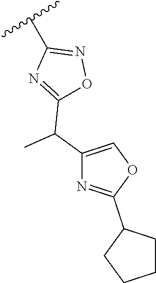 [SMILES: *c1noc(C(C)c2coc(C3CCCC3)n2)n1]

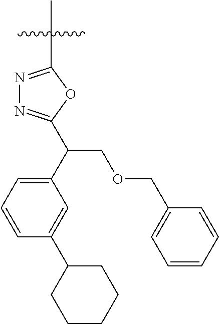 [SMILES: *c1nnc(C(COCc2ccccc2)c2cccc(C3CCCCC3)c2)o1]

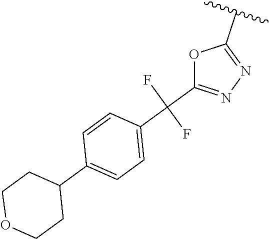 [SMILES: *c1nnc(C(F)(F)c2ccc(C3CCOCC3)cc2)o1]

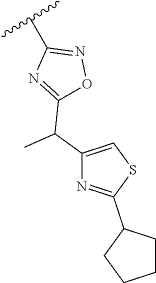 [SMILES: *c1noc(C(C)c2csc(C3CCCC3)n2)n1]

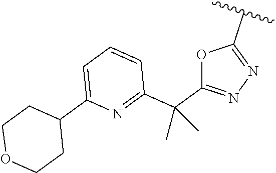 [SMILES: *c1nnc(C(C)(C)c2cccc(C3CCOCC3)n2)o1]

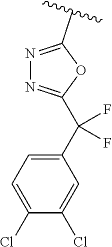 [SMILES: *c1nnc(C(F)(F)c2ccc(Cl)c(Cl)c2)o1]

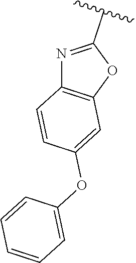 [SMILES: *c1nc2ccc(Oc3ccccc3)cc2o1]

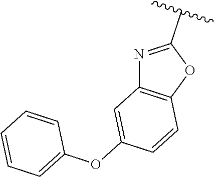 [SMILES: *c1nc2cc(Oc3ccccc3)ccc2o1]

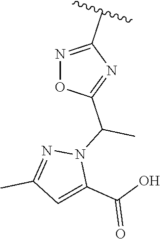 [SMILES: *c1noc(C(C)n2nc(C)cc2C(=O)O)n1]

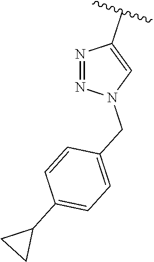 [SMILES: *c1cn(Cc2ccc(C3CC3)cc2)nn1]

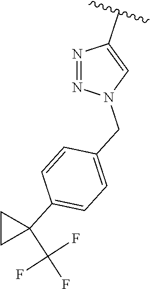 [SMILES: *c1cn(Cc2ccc(C3(C(F)(F)F)CC3)cc2)nn1]

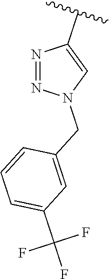 [SMILES: *c1cn(Cc2cccc(C(F)(F)F)c2)nn1]

As defined generally above, R^{B} is a hydrogen, an optionally substituted C₁₋₆ aliphatic group, or a halogen. In some embodiments, R^{B} is a hydrogen. In some embodiments, R^{B} is an optionally substituted C₁₋₆ aliphatic group or a halogen. In some embodiments, R^{B} is an optionally substituted C₁₋₆ aliphatic group. In some embodiments, R^{B} is an optionally substituted methyl group. In some embodiments, R^{B} is a methyl group. In some embodiments, R^{B} is a halogen. In some embodiments, R^{B} is a F. In some embodiments, R^{B} is selected from those depicted in the compounds of Table 8, below.

In some embodiments, R^{A} and R^{B} are geminally attached to the same carbon.

As defined generally above, L⁴ is optionally substituted phenylene, an optionally substituted bivalent 5-6 membered monocyclic heteroarylene ring (having 1-4 heteroatoms independently selected from nitrogen, oxygen, and sulfur), or an optionally substituted bivalent 8-10 membered bicyclic heteroarylene ring (having 1-5 heteroatoms independently selected from nitrogen, oxygen, and sulfur).

In some embodiments, L⁴ is an optionally substituted phenylene. In some embodiments, L⁴ is an optionally substituted bivalent 5-6 membered monocyclic heteroarylene ring (having 1-4 heteroatoms independently selected from nitrogen, oxygen, and sulfur). In certain embodiments, L⁴ is an optionally substituted 5 membered monocyclic heteroarylene ring (having 1-4 heteroatoms independently selected from nitrogen, oxygen, and sulfur). In certain embodiments, L⁴ is an optionally substituted 6 membered monocyclic heteroarylene ring (having 1-4 heteroatoms independently selected from nitrogen, oxygen, and sulfur). In some embodiments, L⁴ is an optionally substituted bivalent 8-10 membered bicyclic heteroarylene ring (having 1-5 heteroatoms independently selected from nitrogen, oxygen, and sulfur).

In some embodiments, L⁴ is isoxazolylene, oxadiazolylene, 1,2,4-oxadiazolylene, oxazolylene, 1,3,4-oxadiazolylene, 4H-1,2,4-triazolylene, 1,2,3-triazolylene, phenylene, pyrrolylene, furanylene, thiopheneylene, pyridinylene, pyrazinylene, pyrimidinylene, pyridazinyl, thiadiazolylene, 1,3,4-thiadiazolylene, thiazolylene, isothiazolylene, or benzo[d]oxazolylene.

In some embodiments, L⁴ is a substituent of Table 2 below, wherein the [symbol] on the left signifies the [symbol] in

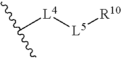

(i.e., the point of attachment of R^{A} to the 2,6-diazaspiro[3.4]octane moiety of Formula I) and the [symbol] on the right signifies the point of attachment of L⁴ onto L⁵. In some embodiments, L⁴ is selected from those depicted in the compounds of Table 8, below. In some embodiments, L⁴ is selected from those depicted in Table 2.

TABLE 2
Exemplary L⁴ substituents

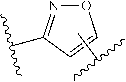

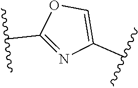 [SMILES: *c1coc(*)n1]

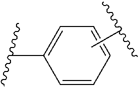

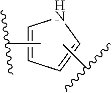

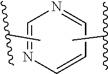

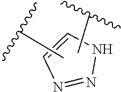

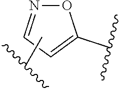

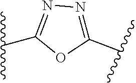 [SMILES: *c1nnc(*)o1]

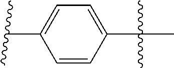 [SMILES: *c1ccc(*)cc1]

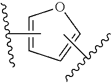

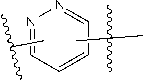

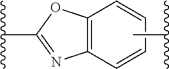

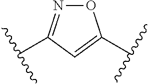 [SMILES: *c1cc(*)on1]

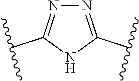 [SMILES: *c1nnc(*)[nH]1]

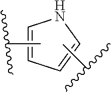

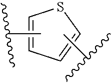

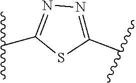 [SMILES: *c1nnc(*)s1]

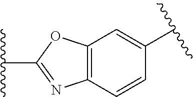 [SMILES: *c1ccc2nc(*)oc2c1]

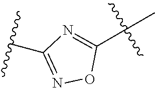 [SMILES: *c1noc(*)n1]

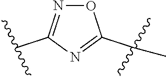 [SMILES: *c1noc(*)n1]

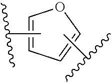

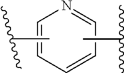

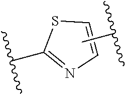

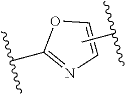

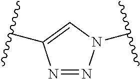 [SMILES: *c1cn(*)nn1]

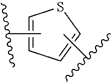

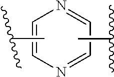

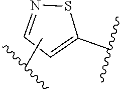

As defined generally above, L⁵ is a covalent bond or a saturated or unsaturated, straight or branched, optionally substituted bivalent C₁₋₄ hydrocarbon chain, wherein 0-2 methylene units of L⁵ are independently replaced by —O—, —NR—, —S—, —C(R)₂—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy²-, or —NRC(O)NR—. In some embodiments, L⁵ is a covalent bond. In some embodiments, L⁵ is a saturated or unsaturated, straight or branched, optionally substituted bivalent C₁₋₄ hydrocarbon chain, wherein 0-2 methylene units of L⁵ are independently replaced by —O—, —NR—, —S—, —C(R)₂—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L⁵ is selected from the group consisting of —CH₂—, —C(CH₃)H—, —NH— —C(O)—, —NH—, —CH₂CH₂—, —CF₂—, —C(CH₃)₂—, —CH₂O—, —OCH₂—, —C(O)O—CH₂—, —C(O)NH—, and

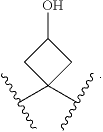 [SMILES: *C1(*)CC(O)C1]

In some embodiments, L⁵ is

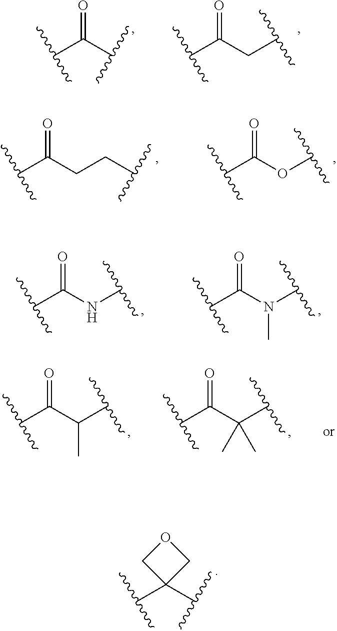

In some embodiments, L⁵ is

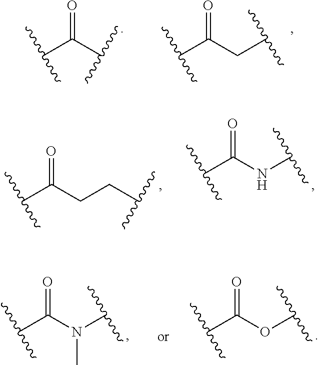

In some embodiments, L⁵ is

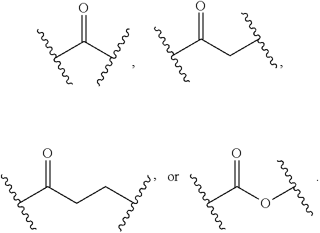

In some embodiments, L⁵ is

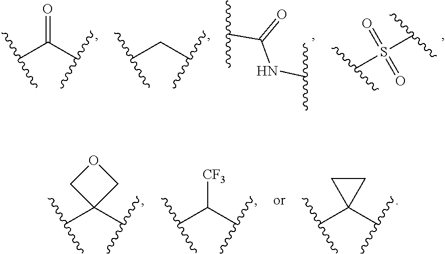

In some embodiments, L⁵ is

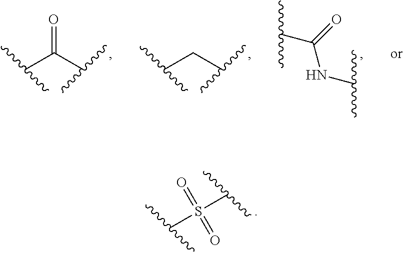

In some embodiments, L⁵ is

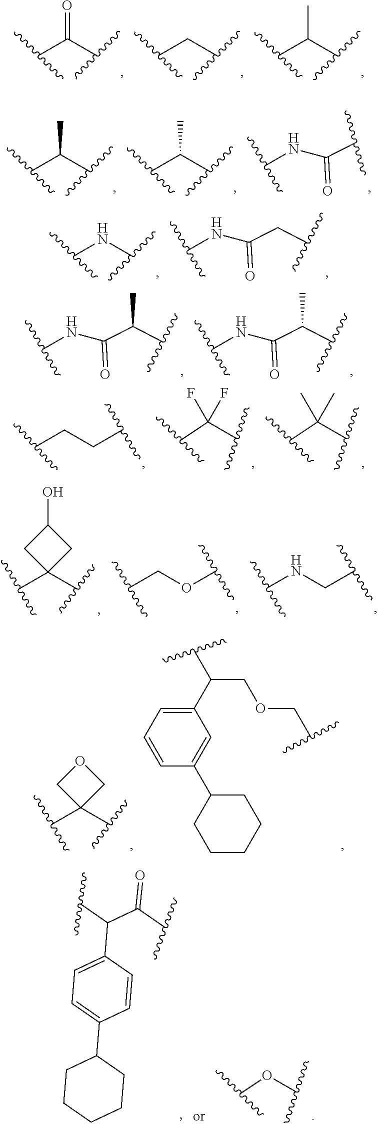

In some embodiments, L⁵ is a substituent depicted in the compounds of Table 8 below.

In some embodiments, the [symbol] on the left of L⁵ signifies the point of attachment to L⁴ and the [symbol] on the right of L⁵ signifies the point of attachment to R¹⁰.

As defined generally above, L² is a covalent bond, a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —C(R)₂—, —NRS(O)₂—, —S(O)₂NR—, —NRC(O)—, —C(O)NR—, —OC(O)NR—, —NRC(O)O—, or —NRC(O)NR—.

In some embodiments, L² is a saturated or unsaturated, straight or branched, optionally substituted bivalent C₁ hydrocarbon chain, wherein 0-1 methylene units of L² are independently replaced by —O—, —NR—, —S—, —C(O)—, —S(O)—, —S(O)₂—, or —C(S)—.

In some embodiments, L² is a covalent bond. In some embodiments, L² is a saturated or unsaturated, straight or branched, optionally substituted bivalent C₁₋₄ hydrocarbon chain, wherein 0-2 methylene units of L² are independently replaced by —C(O)O—, —C(O)—, or —C(O)NR—. In some embodiments, L² is a C₁₋₄ alkylene chain, wherein 1-2 methylene units of L² are independently replaced by —C(O)O—, —C(O)—, or —C(O)NR—. In some embodiments, L² is C₁₋₄ alkylene chain, wherein 1 methylene unit of L² is replaced by —C(O)O—, —C(O)—, or —C(O)NR—. In some embodiments, L² is a saturated optionally substituted bivalent C₁₋₄ hydrocarbon chain. In some embodiments, L² is a saturated bivalent C₁₋₄ hydrocarbon chain, substituted on a single methylene unit by two substituents, which together with the intervening carbon atom form a 3-7 membered carbocyclic ring or heterocyclic ring (having 1-2 heteroatoms independently selected from nitrogen, oxygen, and sulfur). In some embodiments, L² is

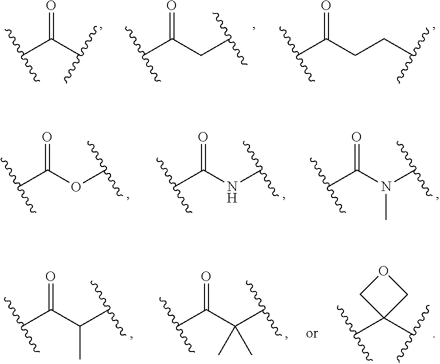

In some embodiments,

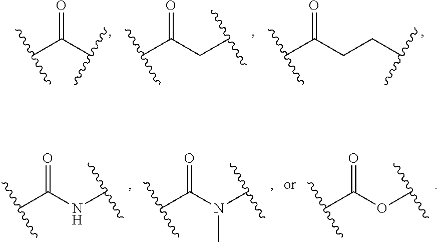

In some embodiments, L² is

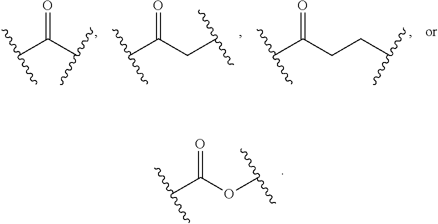

In some embodiments, L² is

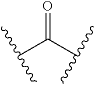 [SMILES: *C(*)=O]

or —CH₂—. In some embodiments, L² is selected from those depicted in the compounds of Table 8, below.

As defined generally above, R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷.

In some embodiments, R⁶ is an optionally substituted C₁₋₆ aliphatic group. In some embodiments, R⁶ is an optionally substituted methyl, ethyl, isopropyl, or tert-butyl group.

In some embodiments, R⁶ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷. In some embodiments, R⁶ is a 3-8 membered saturated or partially unsaturated monocyclic carbocyclic ring, optionally substituted with one or more instances of R⁷. In some embodiments, R⁶ is a phenyl group, optionally substituted with one or more instances of R⁷. In some embodiments, R⁶ is a cyclic group selected from cyclopropyl, cyclobutyl, cyclohexyl and phenyl, wherein the cyclic group is optionally substituted with one or more instances of R⁷. In some embodiments, R⁶ is a cyclopropyl group, optionally substituted with one or more instances of R⁷. In some embodiments, R⁶ is selected from those depicted in the compounds of Table 8, below.

As defined generally above, each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy¹ group, or Cy¹. In some embodiments, each instance of R⁷ is independently halogen, —OR, —CN, an optionally substituted C₁₋₆ aliphatic group, an optionally substituted C₁₋₆ aliphatic-Cy¹ group, or Cy¹. In some embodiments, each instance of R⁷ is independently —F, methyl, ethyl, isopropyl, isobutyl, —CN, optionally substituted phenyl, optionally substituted benzyl, —CF₃, —CH₂OH, —CH₂OCH₃, —CH₂CH₂OCH₃, —CH₂CH₂F, cyclopropyl or —CH₂-(cyclopropyl). In some embodiments, each instance of R⁷ is independently a C₁₋₆ aliphatic group.

In some embodiments, the R⁶ is a cyclic group substituted with 1 instance of R⁷. In some embodiments, the R⁶ is a cyclic group substituted with 2 instances of R⁷. In some embodiments, the R⁶ is a cyclic group substituted with 3 instances of R⁷. In some embodiments, the R⁶ is a cyclic group substituted with 4 instances of R⁷. In some embodiments, the R⁶ is a cyclic group substituted with 5 instances of R⁷.

In some embodiments, -L²-R⁶ is a substituent of Table 3 or Table 4. In some embodiments, -L²-R⁶ or R⁶ is a substituent of Table 4. In some embodiments, -L²-R⁶ is selected from those depicted in the compounds of Table 8, below.

TABLE 3
Exemplary —L²—R⁶ substituents

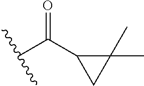 [SMILES: *C(=O)C1CC1(C)C]

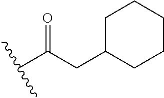 [SMILES: *C(=O)CC1CCCCC1]

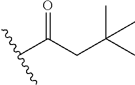 [SMILES: *C(=O)CC(C)(C)C]

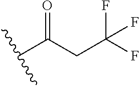 [SMILES: *C(=O)CC(F)(F)F]

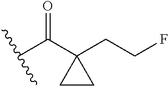 [SMILES: *C(=O)C1(CCF)CC1]

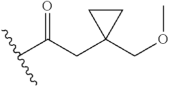 [SMILES: *C(=O)CC1(COC)CC1]

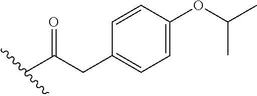 [SMILES: *C(=O)Cc1ccc(OC(C)C)cc1]

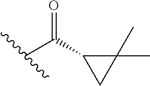 [SMILES: *C(=O)[C@H]1CC1(C)C]

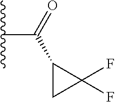 [SMILES: *C(=O)[C@H]1CC1(F)F]

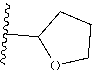 [SMILES: *C1CCCO1]

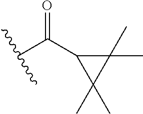 [SMILES: *C(=O)C1C(C)(C)C1(C)C]

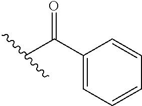 [SMILES: *C(=O)c1ccccc1]

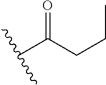 [SMILES: *C(=O)CCC]

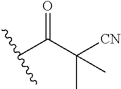 [SMILES: *C(=O)C(C)(C)C#N]

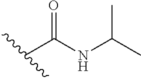 [SMILES: *C(=O)NC(C)C]

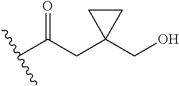 [SMILES: *C(=O)CC1(CO)CC1]

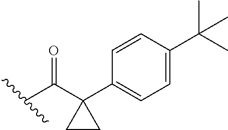 [SMILES: *C(=O)C1(c2ccc(C(C)(C)C)cc2)CC1]

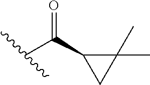 [SMILES: *C(=O)[C@@H]1CC1(C)C]

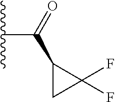 [SMILES: *C(=O)[C@@H]1CC1(F)F]

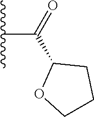 [SMILES: *C(=O)[C@@H]1CCCO1]

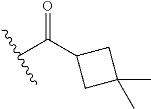 [SMILES: *C(=O)C1CC(C)(C)C1]

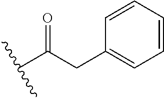 [SMILES: *C(=O)Cc1ccccc1]

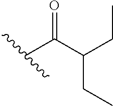 [SMILES: *C(=O)C(CC)CC]

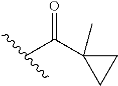 [SMILES: *C(=O)C1(C)CC1]

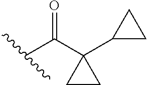 [SMILES: *C(=O)C1(C2CC2)CC1]

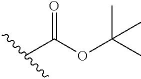 [SMILES: *C(=O)OC(C)(C)C]

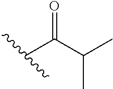 [SMILES: *C(=O)C(C)C]

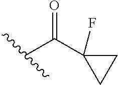 [SMILES: *C(=O)C1(F)CC1]

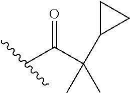 [SMILES: *C(=O)C(C)(C)C1CC1]

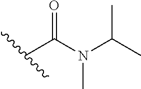 [SMILES: *C(=O)N(C)C(C)C]

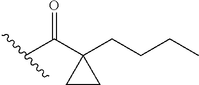 [SMILES: *C(=O)C1(CCCC)CC1]

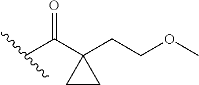 [SMILES: *C(=O)C1(CCOC)CC1]

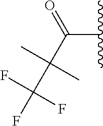 [SMILES: *C(=O)C(C)(C)C(F)(F)F]

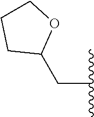 [SMILES: *CC1CCCO1]

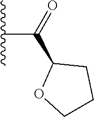 [SMILES: *C(=O)[C@H]1CCCO1]

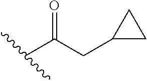 [SMILES: *C(=O)CC1CC1]

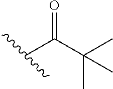 [SMILES: *C(=O)C(C)(C)C]

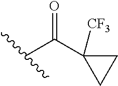 [SMILES: *C(=O)C1(C(F)(F)F)CC1]

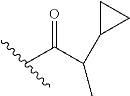 [SMILES: *C(=O)C(C)C1CC1]

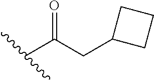 [SMILES: *C(=O)CC1CCC1]

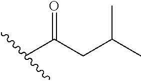 [SMILES: *C(=O)CC(C)C]

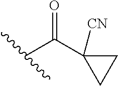 [SMILES: *C(=O)C1(C#N)CC1]

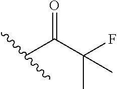 [SMILES: *C(=O)C(C)(C)F]

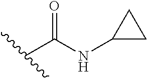 [SMILES: *C(=O)NC1CC1]

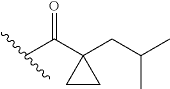 [SMILES: *C(=O)C1(CC(C)C)CC1]

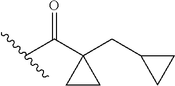 [SMILES: *C(=O)C1(CC2CC2)CC1]

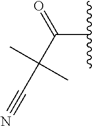 [SMILES: *C(=O)C(C)(C)C#N]

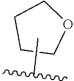

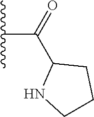 [SMILES: *C(=O)C1CCCN1]

TABLE 4
Exemplary —L²—R⁶ or R⁶ substituents

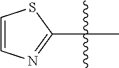 [SMILES: *c1nccs1]

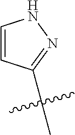 [SMILES: *c1cc[nH]n1]

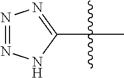 [SMILES: *c1nnn[nH]1]

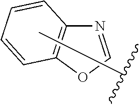

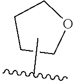

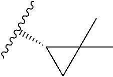 [SMILES: *[C@H]1CC1(C)C]

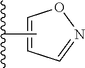

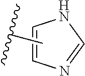

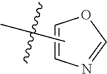

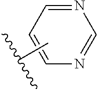

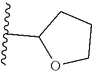 [SMILES: *C1CCCO1]

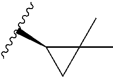 [SMILES: *[C@@H]1CC1(C)C]

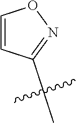 [SMILES: *c1ccon1]

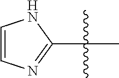 [SMILES: *c1ncc[nH]1]

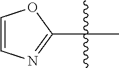 [SMILES: *c1ncco1]

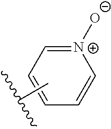

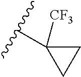 [SMILES: *C1(C(F)(F)F)CC1]

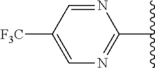 [SMILES: *c1ncc(C(F)(F)F)cn1]

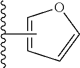

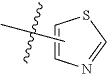

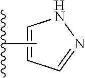

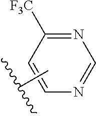

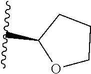 [SMILES: *[C@H]1CCCO1]

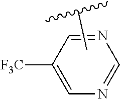

In some embodiments, -L²-R⁶ is

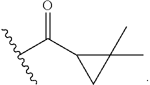 [SMILES: *C(=O)C1CC1(C)C]

In some embodiments, L² is —C(O)— and R⁶ is a cyclopropyl group substituted with —CF₃. In some such embodiments, R⁶ is

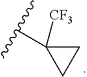 [SMILES: *C1(C(F)(F)F)CC1]

As defined generally above, L³ is a covalent bond, a saturated or unsaturated, straight or branched, optionally substituted bivalent C₁₋₄ hydrocarbon chain, wherein 0-4 methylene units of L³ are independently replaced by —O—, —NR—, —S—, —OC(O)—, —C(O)O—, —C(O)—, —S(O)—, —S(O)₂—, —C(S)—, —C(R)₂—, —NRS(O)₂—, —S(O)₂NR—, —NRC(O)—, —C(O)NR—, —OC(O)NR—, —NRC(O)O—, —NRC(O)NR—, or -Cy²-.

In some embodiments, L³ is a covalent bond. In some embodiments, L³ is a saturated or unsaturated, straight or branched, optionally substituted bivalent C₁₋₄ hydrocarbon chain, wherein 0-4 methylene units of L³ are independently replaced by —S(O)₂—, —C(O)NR—, -Cy²-, or —C(O)—. In some embodiments, L³ is a C₁₋₄ alkylene chain, wherein 1-2 methylene units of L³ are independently replaced by —S(O)₂—, —C(O)NR—, -Cy²-, or —C(O)—. In some embodiments, L³ is C₁₋₄ alkylene chain, wherein 1 methylene unit of L³ is replaced by —S(O)₂—, —C(O)NR—, or —C(O)—. In some embodiments, L³ is a saturated or unsaturated, straight or branched, optionally substituted bivalent C₁₋₄ alkylene chain, wherein 0-2 methylene units of L³ are independently replaced by —C(O)O—, or —C(O)—. In some embodiments, L³ is a C₁₋₄ alkylene chain, wherein 1-2 methylene units of L³ are independently replaced by —C(O)O—, or —C(O)—. In some embodiments, L³ is C₁₋₄ alkylene chain, wherein 1 methylene unit of L³ is replaced by —C(O)O—, or —C(O)—. In some embodiments, L³ is a saturated optionally substituted bivalent C₁₋₄ hydrocarbon chain. In some embodiments, L³ is a saturated bivalent C₁₋₄ hydrocarbon chain, substituted on a single methylene unit by two substituents, which together with the intervening carbon atom form a 3-7 membered carbocyclic ring or heterocyclic ring (having 1-2 heteroatoms independently selected from nitrogen, oxygen, and sulfur). In some embodiments, L³ is

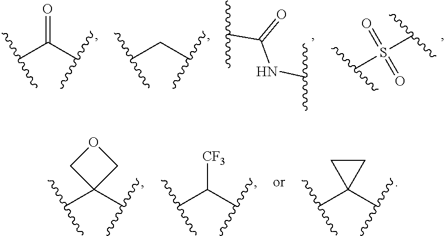

In some embodiments, L³ is

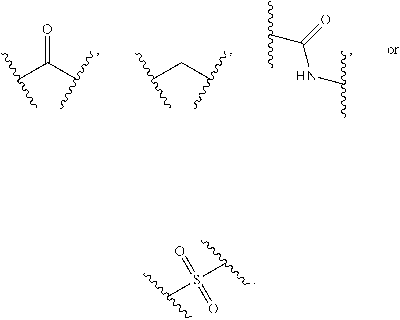

In some embodiments, L³ is

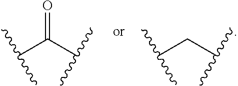

In some embodiments, L³ is selected from those depicted in the compounds of Table 8, below.

As defined generally above, R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments, R⁸ is a cyclic group selected from a 3-8 membered saturated or partially unsaturated monocyclic heterocyclic ring (having 1-2 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments, R⁸ is a 3-8 membered saturated or partially unsaturated monocyclic heterocyclic ring (having 1-2 heteroatoms independently selected from nitrogen, oxygen, and sulfur), optionally substituted with one or more instances of R⁹. In some embodiments, R⁸ is a 5-6 membered monocyclic heteroaromatic ring (having 1-4 heteroatoms independently selected from nitrogen, oxygen, and sulfur), optionally substituted with one or more instances of R⁹. In some embodiments, R⁸ is an 8-10 membered bicyclic heteroaromatic ring (having 1-5 heteroatoms independently selected from nitrogen, oxygen, and sulfur), optionally substituted with one or more instances of R². In some embodiments, R⁸ is a cyclic group selected from pyrazolyl, oxazolyl, thiazolyl, pyrrolidinyl, tetrahydropyranyl, pyridinyl, imidazolyl, indolyl, 1,2,4-triazolyl, 1,2,4-thiadiazolyl, piperidinyl, and indazolyl, wherein the cyclic group is optionally substituted with one or more instances of R⁹. In some embodiments, R⁸ is a pyrazolyl or thiazolyl group, optionally substituted with one or more instances of R⁹. In some embodiments, R⁸ is a pyrazolyl or thiazolyl group. In some embodiments, R⁸ is phenyl, optionally substituted with one or more instances of R⁹. In some embodiments, R⁸ is phenyl, optionally substituted with one or more instances of R⁹, wherein one instance of R⁹ is C₁₋₆-Cy¹. In some embodiments, R⁸ is selected from those depicted in the compounds of Table 8, below. In some embodiments, R⁸ is selected from Table 6, below.

In some embodiments, the R⁸ is a cyclic group substituted with 1 instance of R⁹. In some embodiments, the R⁸ is a cyclic group substituted with 2 instances of R⁹. In some embodiments, the R⁸ is a cyclic group substituted with 3 instances of R⁹. In some embodiments, the R⁸ is a cyclic group substituted with 4 instances of R⁹. In some embodiments, the R⁸ is a cyclic group substituted with 5 instances of R⁹.

As defined generally above, each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy¹ group, or Cy¹.

In some embodiments, each instance of R⁹ is independently halogen, an optionally substituted C₁₋₆ aliphatic group, an optionally substituted C₁₋₆ aliphatic-Cy¹ group, or Cy¹. In some embodiments, each instance of R⁹ is independently an optionally substituted C₁₋₆ aliphatic-Cy¹ group, wherein the Cy¹ is an optionally substituted group selected from phenyl, cyclohexyl, pyridinyl, piperidinyl, cyclopropyl, or tetrahydropyranyl. In some embodiments, R⁹ is a benzylic group. In some embodiments, each instance of R⁹ is independently halogen or an optionally substituted C₁₋₆ aliphatic group. In some embodiments, R⁹ is selected from those depicted in the compounds of Table 8, below.

In some embodiments, -L³-R⁸ is a substituent of Table 5. In some embodiments, -L³-R⁸ or R⁸ is a substituent of Table 6.

TABLE 5
Exemplary —L³—R⁸ substituents

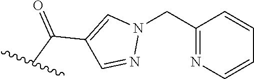 [SMILES: *C(=O)c1cnn(Cc2ccccn2)c1]

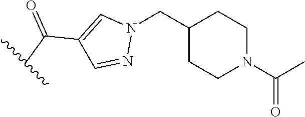 [SMILES: *C(=O)c1cnn(CC2CCN(C(C)=O)CC2)c1]

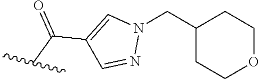 [SMILES: *C(=O)c1cnn(CC2CCOCC2)c1]

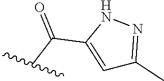 [SMILES: *C(=O)c1cc(C)n[nH]1]

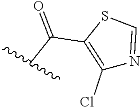 [SMILES: *C(=O)c1scnc1Cl]

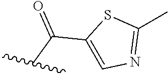 [SMILES: *C(=O)c1cnc(C)s1]

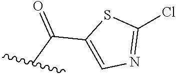 [SMILES: *C(=O)c1cnc(Cl)s1]

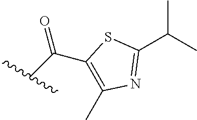 [SMILES: *C(=O)c1sc(C(C)C)nc1C]

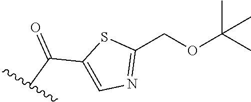 [SMILES: *C(=O)c1cnc(COC(C)(C)C)s1]

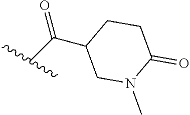 [SMILES: *C(=O)C1CCC(=O)N(C)C1]

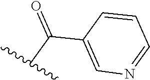 [SMILES: *C(=O)c1cccnc1]

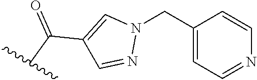 [SMILES: *C(=O)c1cnn(Cc2ccncc2)c1]

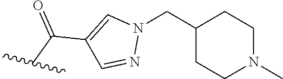 [SMILES: *C(=O)c1cnn(CC2CCN(C)CC2)c1]

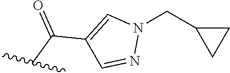 [SMILES: *C(=O)c1cnn(CC2CC2)c1]

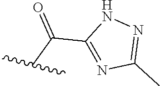 [SMILES: *C(=O)c1nc(C)n[nH]1]

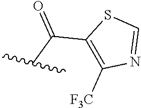 [SMILES: *C(=O)c1scnc1C(F)(F)F]

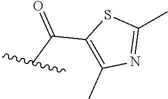 [SMILES: *C(=O)c1sc(C)nc1C]

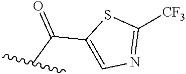 [SMILES: *C(=O)c1cnc(C(F)(F)F)s1]

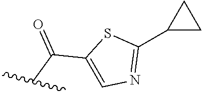 [SMILES: *C(=O)c1cnc(C2CC2)s1]

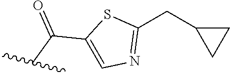 [SMILES: *C(=O)c1cnc(CC2CC2)s1]

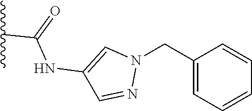 [SMILES: *C(=O)Nc1cnn(Cc2ccccc2)c1]

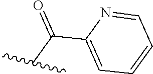 [SMILES: *C(=O)c1ccccn1]

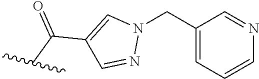 [SMILES: *C(=O)c1cnn(Cc2cccnc2)c1]

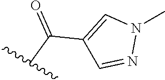 [SMILES: *C(=O)c1cnn(C)c1]

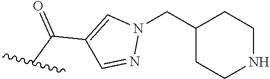 [SMILES: *C(=O)c1cnn(CC2CCNCC2)c1]

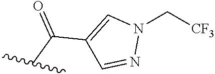 [SMILES: *C(=O)c1cnn(CC(F)(F)F)c1]

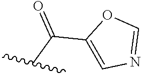 [SMILES: *C(=O)c1cnco1]

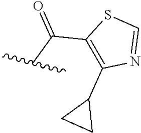 [SMILES: *C(=O)c1scnc1C1CC1]

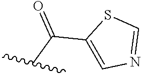 [SMILES: *C(=O)c1cncs1]

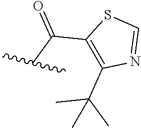 [SMILES: *C(=O)c1scnc1C(C)(C)C]

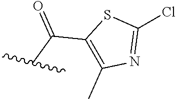 [SMILES: *C(=O)c1sc(Cl)nc1C]

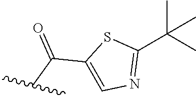 [SMILES: *C(=O)c1cnc(C(C)(C)C)s1]

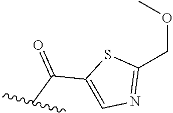 [SMILES: *C(=O)c1cnc(COC)s1]

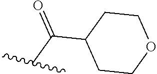 [SMILES: *C(=O)C1CCOCC1]

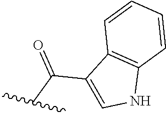 [SMILES: *C(=O)c1c[nH]c2ccccc12]

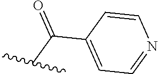 [SMILES: *C(=O)c1ccncc1]

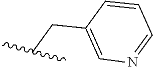 [SMILES: *Cc1cccnc1]

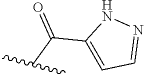 [SMILES: *C(=O)c1ccn[nH]1]

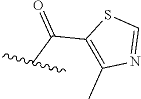 [SMILES: *C(=O)c1scnc1C]

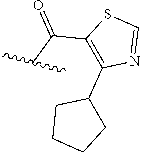 [SMILES: *C(=O)c1scnc1C1CCCC1]

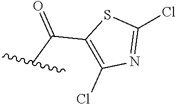 [SMILES: *C(=O)c1sc(Cl)nc1Cl]

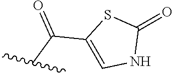 [SMILES: *C(=O)c1c[nH]c(=O)s1]

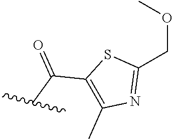 [SMILES: *C(=O)c1sc(COC)nc1C]

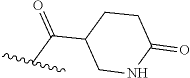 [SMILES: *C(=O)C1CCC(=O)NC1]

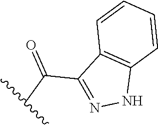 [SMILES: *C(=O)c1n[nH]c2ccccc12]

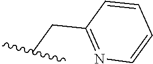 [SMILES: *Cc1ccccn1]

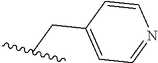 [SMILES: *Cc1ccncc1]

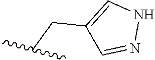 [SMILES: *Cc1cn[nH]c1]

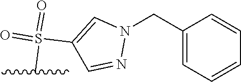 [SMILES: *S(=O)(=O)c1cnn(Cc2ccccc2)c1]

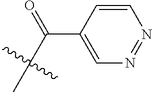 [SMILES: *C(=O)c1ccnnc1]

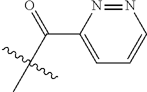 [SMILES: *C(=O)c1cccnn1]

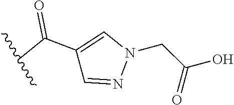 [SMILES: *C(=O)c1cnn(CC(=O)O)c1]

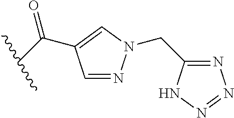 [SMILES: *C(=O)c1cnn(Cc2nnn[nH]2)c1]

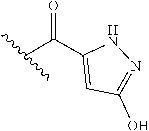 [SMILES: *C(=O)c1cc(O)n[nH]1]

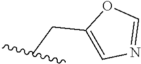 [SMILES: *Cc1cnco1]

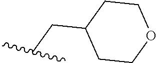 [SMILES: *CC1CCOCC1]

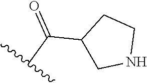 [SMILES: *C(=O)C1CCNC1]

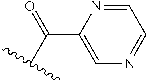 [SMILES: *C(=O)c1cnccn1]

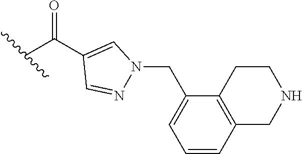 [SMILES: *C(=O)c1cnn(Cc2cccc3c2CCNC3)c1]

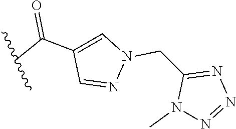 [SMILES: *C(=O)c1cnn(Cc2nnnn2C)c1]

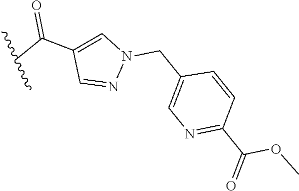 [SMILES: *C(=O)c1cnn(Cc2ccc(C(=O)OC)nc2)c1]

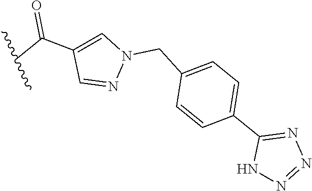 [SMILES: *C(=O)c1cnn(Cc2ccc(-c3nnn[nH]3)cc2)c1]

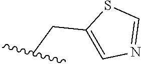 [SMILES: *Cc1cncs1]

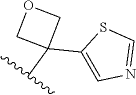 [SMILES: *C1(c2cncs2)COC1]

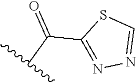 [SMILES: *C(=O)c1nncs1]

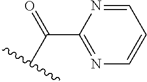 [SMILES: *C(=O)c1ncccn1]

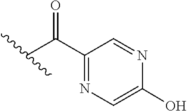 [SMILES: *C(=O)c1cnc(O)cn1]

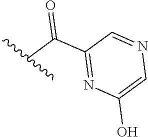 [SMILES: *C(=O)c1cncc(O)n1]

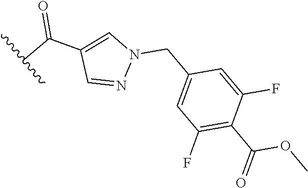 [SMILES: *C(=O)c1cnn(Cc2cc(F)c(C(=O)OC)c(F)c2)c1]

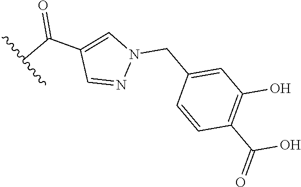 [SMILES: *C(=O)c1cnn(Cc2ccc(C(=O)O)c(O)c2)c1]

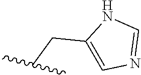 [SMILES: *Cc1cnc[nH]1]

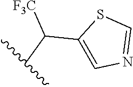 [SMILES: *C(c1cncs1)C(F)(F)F]

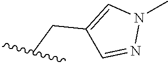 [SMILES: *Cc1cnn(C)c1]

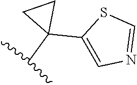 [SMILES: *C1(c2cncs2)CC1]

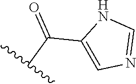 [SMILES: *C(=O)c1cnc[nH]1]

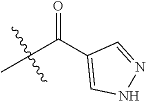 [SMILES: *C(=O)c1cn[nH]c1]

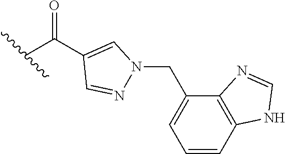 [SMILES: *C(=O)c1cnn(Cc2cccc3[nH]cnc23)c1]

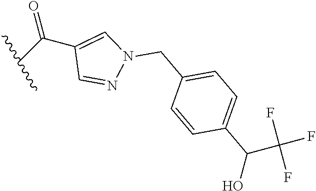 [SMILES: *C(=O)c1cnn(Cc2ccc(C(O)C(F)(F)F)cc2)c1]

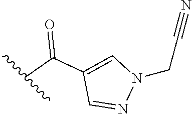 [SMILES: *C(=O)c1cnn(CC#N)c1]

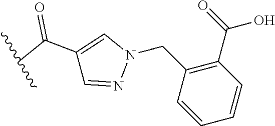 [SMILES: *C(=O)c1cnn(Cc2ccccc2C(=O)O)c1]

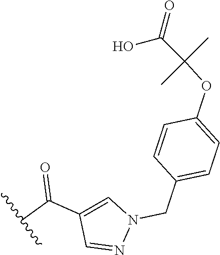 [SMILES: *C(=O)c1cnn(Cc2ccc(OC(C)(C)C(=O)O)cc2)c1]

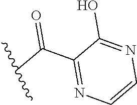 [SMILES: *C(=O)c1nccnc1O]

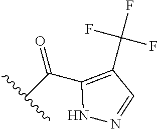 [SMILES: *C(=O)c1[nH]ncc1C(F)(F)F]

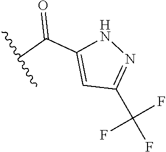 [SMILES: *C(=O)c1cc(C(F)(F)F)n[nH]1]

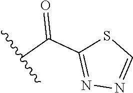 [SMILES: *C(=O)c1nncs1]

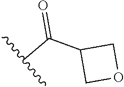 [SMILES: *C(=O)C1COC1]

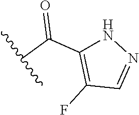 [SMILES: *C(=O)c1[nH]ncc1F]

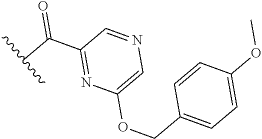 [SMILES: *C(=O)c1cncc(OCc2ccc(OC)cc2)n1]

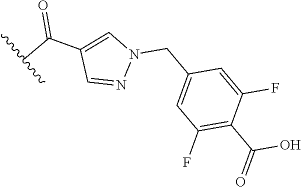 [SMILES: *C(=O)c1cnn(Cc2cc(F)c(C(=O)O)c(F)c2)c1]

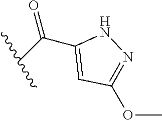 [SMILES: *C(=O)c1cc(OC)n[nH]1]

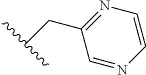 [SMILES: *Cc1cnccn1]

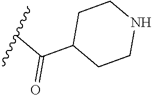 [SMILES: *C(=O)C1CCNCC1]

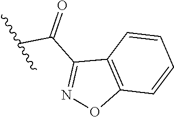 [SMILES: *C(=O)c1noc2ccccc12]

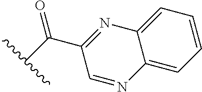 [SMILES: *C(=O)c1cnc2ccccc2n1]

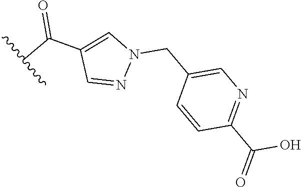 [SMILES: *C(=O)c1cnn(Cc2ccc(C(=O)O)nc2)c1]

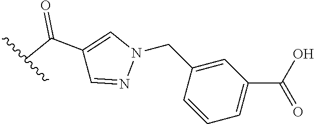 [SMILES: *C(=O)c1cnn(Cc2cccc(C(=O)O)c2)c1]

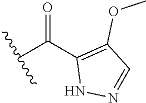 [SMILES: *C(=O)c1[nH]ncc1OC]

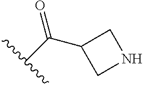 [SMILES: *C(=O)C1CNC1]

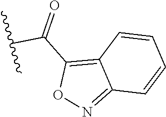 [SMILES: *C(=O)c1onc2ccccc12]

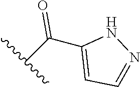 [SMILES: *C(=O)c1ccn[nH]1]

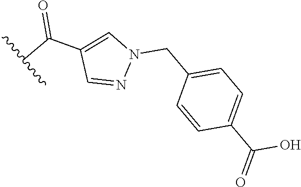 [SMILES: *C(=O)c1cnn(Cc2ccc(C(=O)O)cc2)c1]

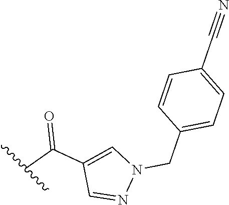 [SMILES: *C(=O)c1cnn(Cc2ccc(C#N)cc2)c1]

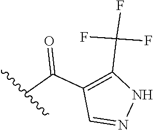 [SMILES: *C(=O)c1cn[nH]c1C(F)(F)F]

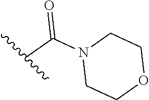 [SMILES: *C(=O)N1CCOCC1]

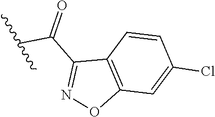 [SMILES: *C(=O)c1noc2cc(Cl)ccc12]

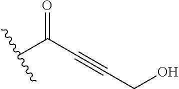 [SMILES: *C(=O)C#CCO]

TABLE 6
Exemplary —L³—R⁸ or R⁸ substituents

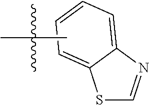

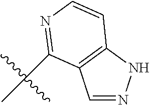 [SMILES: *c1nccc2[nH]ncc12]

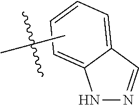

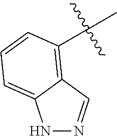 [SMILES: *c1cccc2[nH]ncc12]

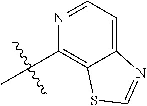 [SMILES: *c1nccc2ncsc12]

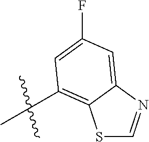 [SMILES: *c1cc(F)cc2ncsc12]

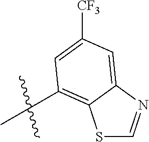 [SMILES: *c1cc(C(F)(F)F)cc2ncsc12]

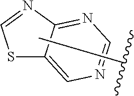

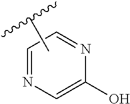

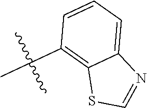 [SMILES: *c1cccc2ncsc12]

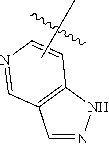

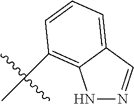 [SMILES: *c1cccc2cn[nH]c12]

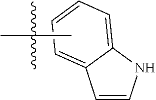

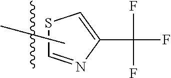

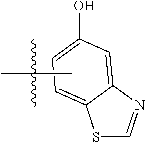

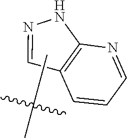

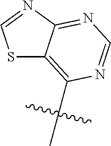 [SMILES: *c1ncnc2ncsc12]

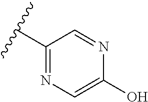 [SMILES: *c1cnc(O)cn1]

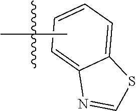

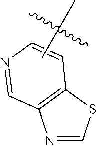

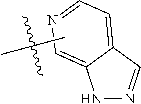

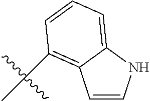 [SMILES: *c1cccc2[nH]ccc12]

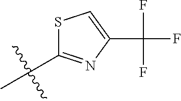 [SMILES: *c1nc(C(F)(F)F)cs1]

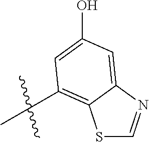 [SMILES: *c1cc(O)cc2ncsc12]

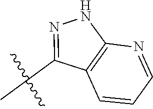 [SMILES: *c1n[nH]c2ncccc12]

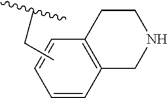

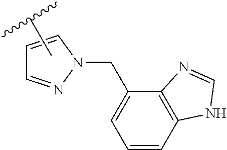

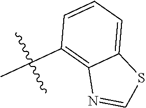 [SMILES: *c1cccc2scnc12]

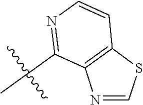 [SMILES: *c1nccc2scnc12]

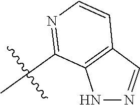 [SMILES: *c1nccc2cn[nH]c12]

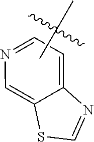

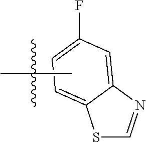

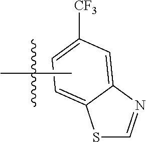

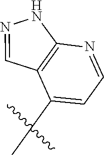 [SMILES: *c1ccnc2[nH]ncc12]

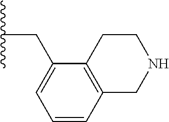 [SMILES: *Cc1cccc2c1CCNC2]

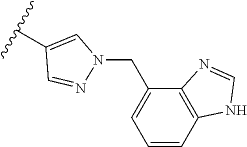 [SMILES: *c1cnn(Cc2cccc3[nH]cnc23)c1]

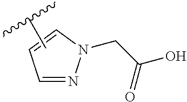

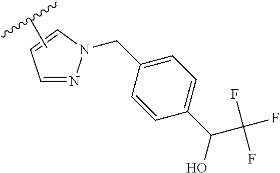

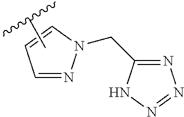

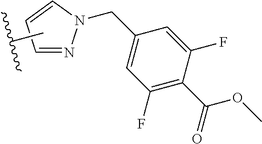

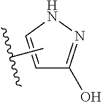

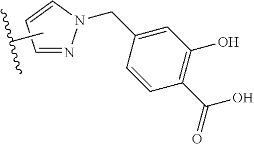

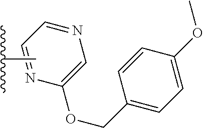

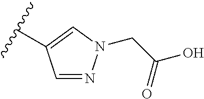 [SMILES: *c1cnn(CC(=O)O)c1]

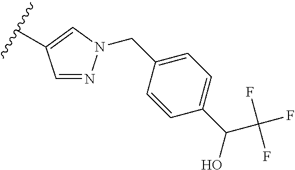 [SMILES: *c1cnn(Cc2ccc(C(O)C(F)(F)F)cc2)c1]

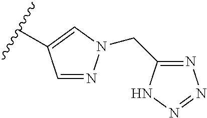 [SMILES: *c1cnn(Cc2nnn[nH]2)c1]

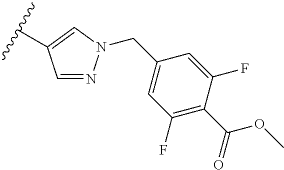 [SMILES: *c1cnn(Cc2cc(F)c(C(=O)OC)c(F)c2)c1]

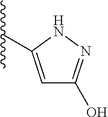 [SMILES: *c1cc(O)n[nH]1]

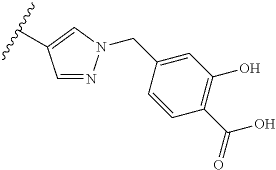 [SMILES: *c1cnn(Cc2ccc(C(=O)O)c(O)c2)c1]

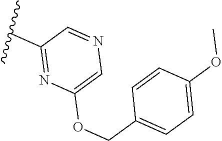 [SMILES: *c1cncc(OCc2ccc(OC)cc2)n1]

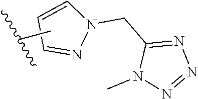

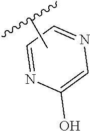

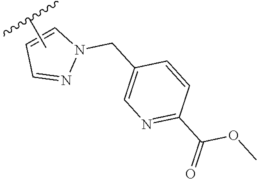

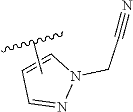

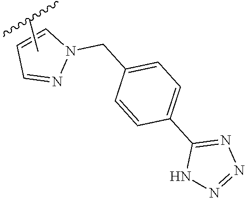

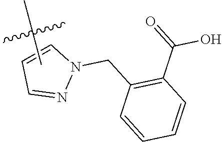

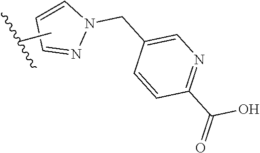

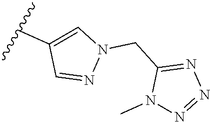 [SMILES: *c1cnn(Cc2nnnn2C)c1]

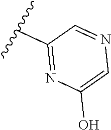 [SMILES: *c1cncc(O)n1]

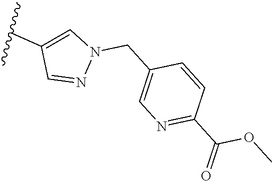 [SMILES: *c1cnn(Cc2ccc(C(=O)OC)nc2)c1]

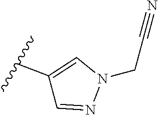 [SMILES: *c1cnn(CC#N)c1]

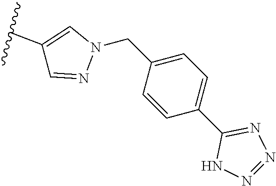 [SMILES: *c1cnn(Cc2ccc(-c3nnn[nH]3)cc2)c1]

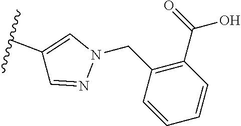 [SMILES: *c1cnn(Cc2ccccc2C(=O)O)c1]

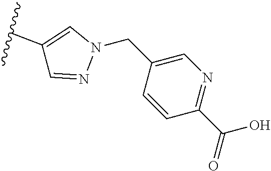 [SMILES: *c1cnn(Cc2ccc(C(=O)O)nc2)c1]

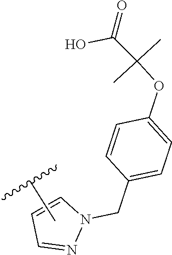

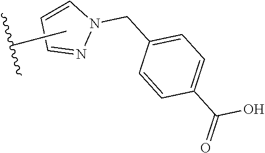

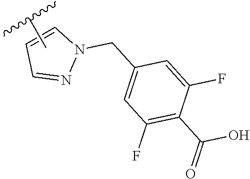

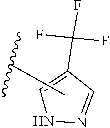

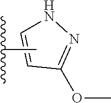

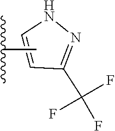

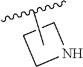

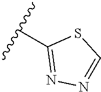 [SMILES: *c1nncs1]

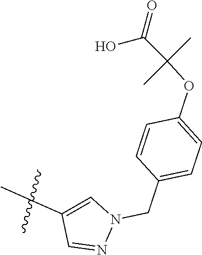 [SMILES: *c1cnn(Cc2ccc(OC(C)(C)C(=O)O)cc2)c1]

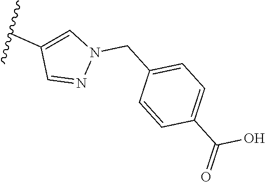 [SMILES: *c1cnn(Cc2ccc(C(=O)O)cc2)c1]

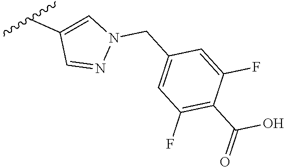 [SMILES: *c1cnn(Cc2cc(F)c(C(=O)O)c(F)c2)c1]

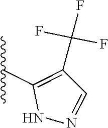 [SMILES: *c1[nH]ncc1C(F)(F)F]

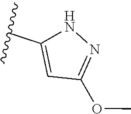 [SMILES: *c1cc(OC)n[nH]1]

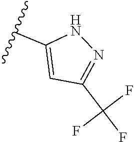 [SMILES: *c1cc(C(F)(F)F)n[nH]1]

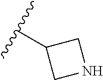 [SMILES: *C1CNC1]

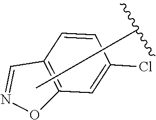

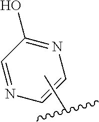

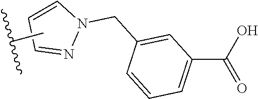

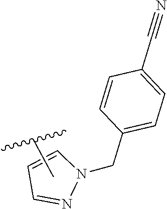

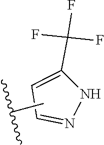

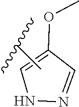

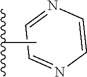

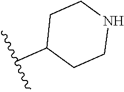 [SMILES: *C1CCNCC1]

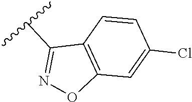 [SMILES: *c1noc2cc(Cl)ccc12]

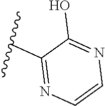 [SMILES: *c1nccnc1O]

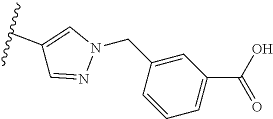 [SMILES: *c1cnn(Cc2cccc(C(=O)O)c2)c1]

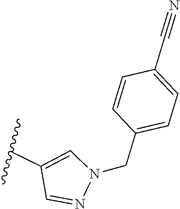 [SMILES: *c1cnn(Cc2ccc(C#N)cc2)c1]

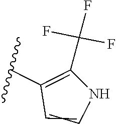 [SMILES: *c1cc[nH]c1C(F)(F)F]

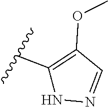 [SMILES: *c1[nH]ncc1OC]

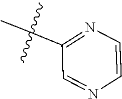 [SMILES: *c1cnccn1]

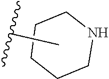

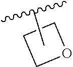

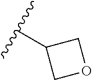 [SMILES: *C1COC1]

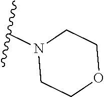 [SMILES: *N1CCOCC1]

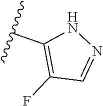 [SMILES: *c1[nH]ncc1F]

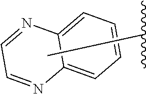

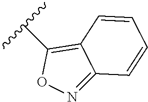 [SMILES: *c1onc2ccccc12]

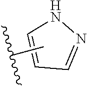

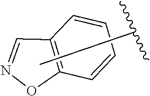

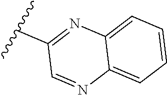 [SMILES: *c1cnc2ccccc2n1]

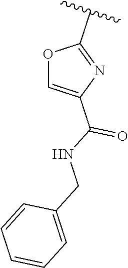 [SMILES: *c1nc(C(=O)NCc2ccccc2)co1]

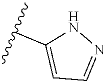 [SMILES: *c1ccn[nH]1]

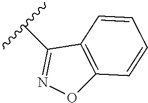 [SMILES: *c1noc2ccccc12]

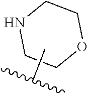

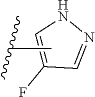

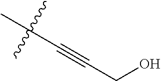 [SMILES: *C#CCO]

In some embodiments, -L³-R⁸ is

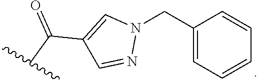 [SMILES: *C(=O)c1cnn(Cc2ccccc2)c1]

In some embodiments, -L³-R⁸ is

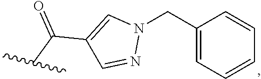

wherein R⁸ is substituted with one or more R⁹, wherein one R⁹ is an optionally substituted C₁₋₆ aliphatic-Cy¹ group. In some such embodiments, R⁹ is an optionally substituted C₁₋₂ aliphatic-Cy¹ group wherein Cy¹ is phenyl.

As defined generally above, R¹⁰ is hydrogen or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, R¹⁰ is hydrogen. In some embodiments, R¹⁰ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, R¹⁰ is a 3-8 membered saturated or partially unsaturated monocyclic carbocyclic ring. In some embodiments, R¹⁰ is a 7-12 membered saturated or partially unsaturated bicyclic carbocyclic ring. In some embodiments, R¹⁰ is phenyl. In some embodiments, R¹⁰ is an 8-10 membered bicyclic aromatic carbocyclic ring. In some embodiments, R¹⁰ is a 3-8 membered saturated or partially unsaturated monocyclic heterocyclic ring (having 1-3 heteroatoms independently selected from nitrogen, oxygen, and sulfur). In some embodiments, R¹⁰ is a 7-12 membered saturated or partially unsaturated bicyclic heterocyclic ring (having 1-4 heteroatoms independently selected from nitrogen, oxygen, and sulfur). In some embodiments, R¹⁰ is a 5-6 membered monocyclic heteroaromatic ring (having 1-4 heteroatoms independently selected from nitrogen, oxygen, and sulfur). In some embodiments, R¹⁰ is an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, the R¹⁰ is a cyclic group substituted with 1 instance of R⁹. In some embodiments, the R¹⁰ is a cyclic group substituted with 2 instances of R⁹. In some embodiments, the R¹⁰ is a cyclic group substituted with 3 instances of R⁹. In some embodiments, the R¹⁰ is a cyclic group substituted with 4 instances of R⁹. In some embodiments, the R¹⁰ is a cyclic group substituted with 5 instances of R⁹.

In some embodiments, R¹⁰ is selected from those depicted in the compounds of Table 8, below.

In some embodiments, R¹⁰ is a substituent of Table 7.

TABLE 7
Exemplary R¹⁰ substituents

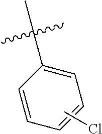

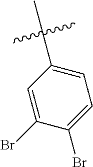 [SMILES: *c1ccc(Br)c(Br)c1]

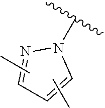

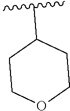 [SMILES: *C1CCOCC1]

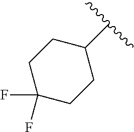 [SMILES: *C1CCC(F)(F)CC1]

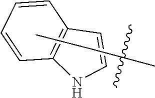

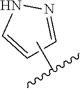

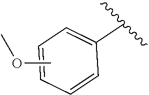

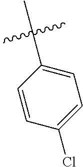 [SMILES: *c1ccc(Cl)cc1]

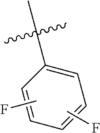

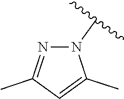

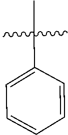 [SMILES: *c1ccccc1]

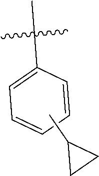

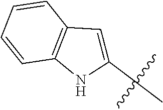 [SMILES: *c1cc2ccccc2[nH]1]

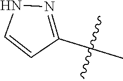 [SMILES: *c1cc[nH]n1]

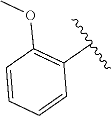 [SMILES: *c1ccccc1OC]

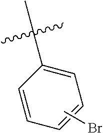

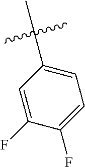 [SMILES: *c1ccc(F)c(F)c1]

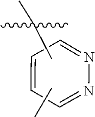

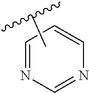

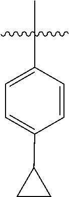 [SMILES: *c1ccc(C2CC2)cc1]

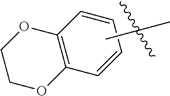

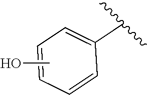

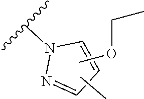

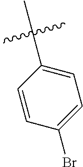 [SMILES: *c1ccc(Br)cc1]

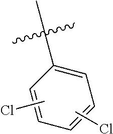

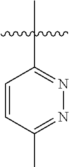 [SMILES: *c1ccc(C)nn1]

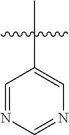 [SMILES: *c1cncnc1]

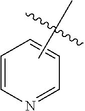

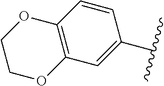 [SMILES: *c1ccc2c(c1)OCCO2]

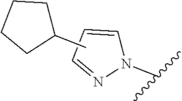

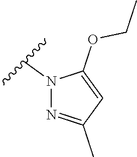 [SMILES: *n1nc(C)cc1OCC]

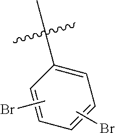

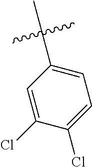 [SMILES: *c1ccc(Cl)c(Cl)c1]

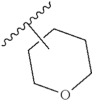

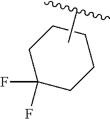

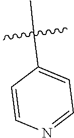 [SMILES: *c1ccncc1]

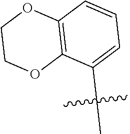 [SMILES: *c1cccc2c1OCCO2]

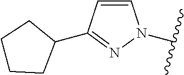 [SMILES: *n1ccc(C2CCCC2)n1]

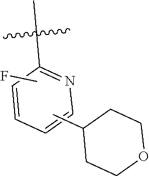

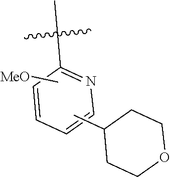

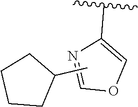

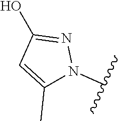 [SMILES: *n1nc(O)cc1C]

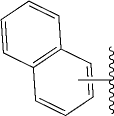

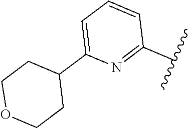 [SMILES: *c1cccc(C2CCOCC2)n1]

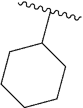 [SMILES: *C1CCCCC1]

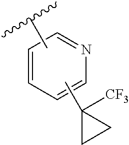

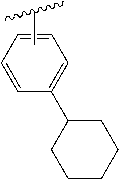

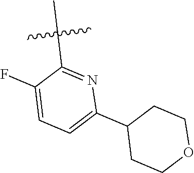 [SMILES: *c1nc(C2CCOCC2)ccc1F]

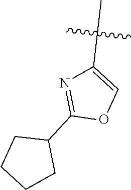 [SMILES: *c1coc(C2CCCC2)n1]

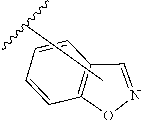

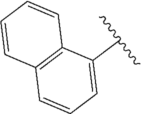 [SMILES: *c1cccc2ccccc12]

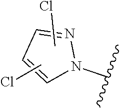

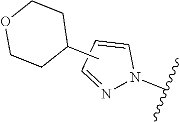

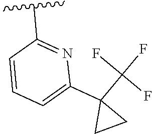 [SMILES: *c1cccc(C2(C(F)(F)F)CC2)n1]

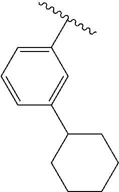 [SMILES: *c1cccc(C2CCCCC2)c1]

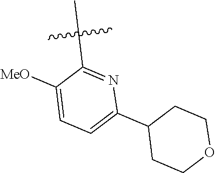 [SMILES: *c1nc(C2CCOCC2)ccc1OC]

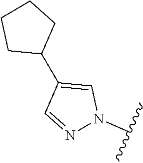 [SMILES: *n1cc(C2CCCC2)cn1]

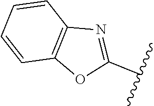 [SMILES: *c1nc2ccccc2o1]

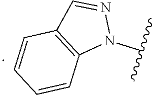 [SMILES: *n1ncc2ccccc21]

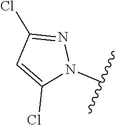 [SMILES: *n1nc(Cl)cc1Cl]

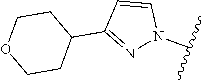 [SMILES: *n1ccc(C2CCOCC2)n1]

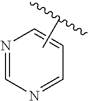

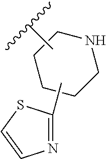

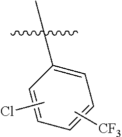

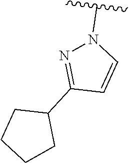 [SMILES: *n1ccc(C2CCCC2)n1]

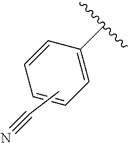

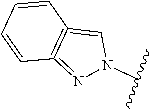

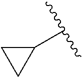 [SMILES: *C1CC1]

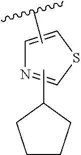

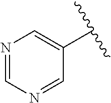 [SMILES: *c1cncnc1]

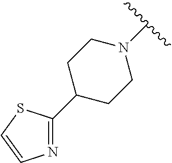 [SMILES: *N1CCC(c2nccs2)CC1]

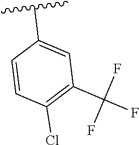 [SMILES: *c1ccc(Cl)c(C(F)(F)F)c1]

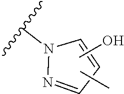

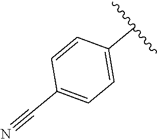 [SMILES: *c1ccc(C#N)cc1]

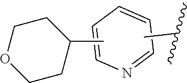

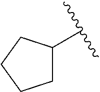 [SMILES: *C1CCCC1]

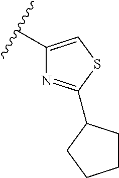 [SMILES: *c1csc(C2CCCC2)n1]

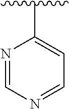 [SMILES: *c1ccncn1]

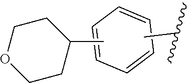

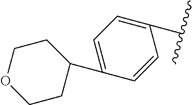 [SMILES: *c1ccc(C2CCOCC2)cc1]

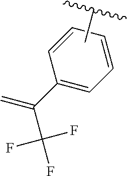

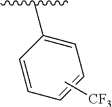

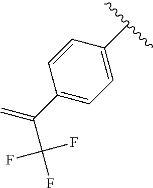 [SMILES: *c1ccc(C(=C)C(F)(F)F)cc1]

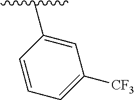 [SMILES: *c1cccc(C(F)(F)F)c1]

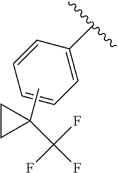

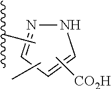

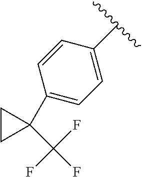 [SMILES: *c1ccc(C2(C(F)(F)F)CC2)cc1]

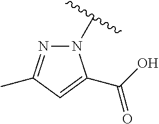 [SMILES: *n1nc(C)cc1C(=O)O]

As defined generally above, each Cy¹ is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur). In some embodiments, each Cy¹ is independently a 3-8 membered saturated or partially unsaturated monocyclic carbocyclic ring or phenyl. In some embodiments, each Cy¹ is independently an optionally substituted cyclic group selected from a 3-8 membered saturated or partially unsaturated monocyclic carbocyclic ring. In some embodiments, Cy¹ is phenyl. In some embodiments, each Cy¹ is independently a 3-8 membered saturated or partially unsaturated monocyclic heterocyclic ring (having 1-2 heteroatoms independently selected from nitrogen, oxygen, and sulfur) or a 5-6 membered monocyclic heteroaromatic ring (having 1-4 heteroatoms independently selected from nitrogen, oxygen, and sulfur). In some embodiments, each Cy¹ is independently a 3-8 membered saturated or partially unsaturated monocyclic heterocyclic ring (having 1-2 heteroatoms independently selected from nitrogen, oxygen, and sulfur). In some embodiments, each Cy¹ is independently a 5-6 membered monocyclic heteroaromatic ring (having 1-4 heteroatoms independently selected from nitrogen, oxygen, and sulfur).

In some embodiments, Cy¹ is selected from those depicted in the compounds of Table 8, below.

As defined generally above, each -Cy²- is independently an optionally substituted and bivalent cyclic group selected from a 3-8 membered saturated or partially unsaturated monocyclic carbocyclene, phenylene, a 3-8 membered saturated or partially unsaturated monocyclic heterocyclene ring (having 1-2 heteroatoms independently selected from nitrogen, oxygen, and sulfur), and a 5-6 membered monocyclic heteroarylene ring (having 1-4 heteroatoms independently selected from nitrogen, oxygen, and sulfur).

In some embodiments, each -Cy²- is independently a 3-8 membered saturated or partially unsaturated monocyclic carbocyclene. In some embodiments, each -Cy²- is independently phenylene. In some embodiments, each -Cy²- is independently a 3-8 membered saturated or partially unsaturated monocyclic heterocyclene ring (having 1-2 heteroatoms independently selected from nitrogen, oxygen, and sulfur). In some embodiments, each -Cy²- is independently a 5-6 membered monocyclic heteroarylene ring (having 1-4 heteroatoms independently selected from nitrogen, oxygen, and sulfur).

As defined generally above, each R is independently hydrogen, halogen, an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), an optionally substituted 5-6 membered heteroaryl ring (having 1-4 heteroatoms independently selected from nitrogen, oxygen, and sulfur), two R groups on the same nitrogen atom or carbon atom are taken together with the nitrogen atom or carbon atom to form an optionally substituted 3-7 membered saturated, partially unsaturated, or heteroaryl ring (having 0-3 heteroatoms independently selected from nitrogen, oxygen, and sulfur), or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur).

In some embodiments, R is hydrogen. In some embodiments, each R is independently halogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur). In some embodiments, R is halogen. In some embodiments, each R is independently an optionally substituted C₁₋₆ aliphatic group. In some embodiments, each R is independently an optionally substituted phenyl. In some embodiments, each R is independently an optionally substituted 3-7 membered saturated or partially unsaturated carbocyclic ring. In some embodiments, each R is independently an optionally substituted 3-7 membered saturated or partially unsaturated heterocyclic ring (having 1-2 heteroatoms independently selected from nitrogen, oxygen, and sulfur). In some embodiments, each R is independently an optionally substituted 5-6 membered heteroaryl ring (having 1-4 heteroatoms independently selected from nitrogen, oxygen, and sulfur).

In some embodiments, two R groups on the same nitrogen atom or carbon atom are taken together with the nitrogen atom or carbon atom to form an optionally substituted 4-7 membered saturated, partially unsaturated, or heteroaryl ring (having 0-3 heteroatoms, in addition to the nitrogen, independently selected from nitrogen, oxygen, and sulfur); or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur). In some embodiments, two R groups on the same nitrogen atom or carbon atom are taken together with the nitrogen atom or carbon atom to form an optionally substituted 4-7 membered saturated, partially unsaturated, or heteroaryl ring (having 0-3 heteroatoms, in addition to the nitrogen, independently selected from nitrogen, oxygen, and sulfur). In some embodiments, two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur). In some embodiments, two R groups on the same nitrogen atom are taken together with the nitrogen atom to form an optionally substituted moiety selected from the group consisting of

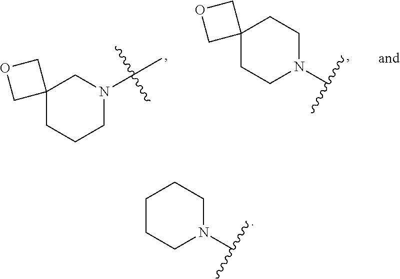 [SMILES: *N1CCCC2(COC2)C1 ; *N1CCC2(CC1)COC2 ; *N1CCCCC1]

In some embodiments, two R groups on the same nitrogen atom are taken together with the nitrogen atom to form

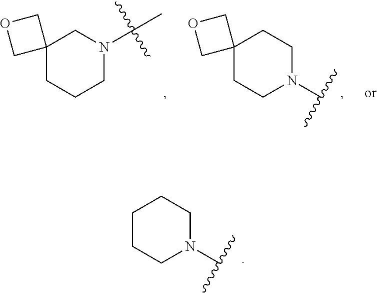 [SMILES: *N1CCCC2(COC2)C1 ; *N1CCC2(CC1)COC2 ; *N1CCCCC1]

In some embodiments, two R groups on the same nitrogen atom are taken together with the nitrogen atom to form optionally substituted

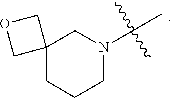 [SMILES: *N1CCCC2(COC2)C1]

In some embodiments, two R groups on the same nitrogen atom are taken together with the nitrogen atom to form

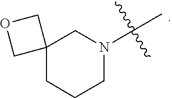 [SMILES: *N1CCCC2(COC2)C1]

In some embodiments, two R groups on the same nitrogen atom are taken together with the nitrogen atom to form optionally substituted

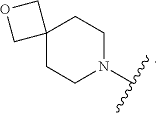 [SMILES: *N1CCC2(CC1)COC2]

In some embodiments, two R groups on the same nitrogen atom are taken together with the nitrogen atom to form

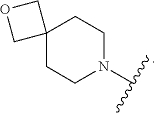 [SMILES: *N1CCC2(CC1)COC2]

In some embodiments, two R groups on the same nitrogen atom are taken together with the nitrogen atom to form optionally substituted

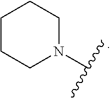 [SMILES: *N1CCCCC1]

In some embodiments, two R groups on the same nitrogen atom are taken together with the nitrogen atom to form

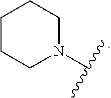 [SMILES: *N1CCCCC1]

In some embodiments, each R is independently selected from those depicted in the compounds of Table 8, below.

In some embodiments, one or both of L² and L³ is a covalent bond. In certain embodiments, L² is a covalent bond and L³ is a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In certain embodiments, L³ is a covalent bond and L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In certain embodiments, L² and L³ are each both a covalent bond. In some embodiments, L² and L³ are selected from those depicted in the compounds of Table 8, below.

In some embodiments, the compound of Formula I is a compound of Formula II:

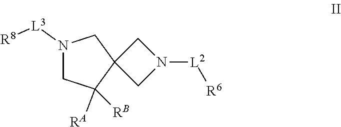

or a pharmaceutically acceptable salt thereof, wherein R^{A}, R^{B}, L², R⁶, L³ and R⁸, and their constituent groups, are each as defined and described herein. In some embodiments, R^{A} is a substituent from Table 1. In some embodiments, -L²-R⁶ is a substituent from Table 3 or Table 4. In some embodiments, -L³-R⁸ is a substituent from Table 5 or Table 6. In some embodiments, R^{A} is a substituent from Table 1, and -L²-R⁶ is a substituent from Table 3 or Table 4. In some embodiments, R^{A} is a substituent from Table 1, and -L³-R⁸ is a substituent from Table 5 or Table 6. In some embodiments, -L²-R⁶ is a substituent from Table 3 or Table 4, and -L³-R⁸ is a substituent from Table 5 or Table 6.

In some embodiments, the compound of Formula I is a compound of Formula IIIa, IIIb, IIIa′, IIIb′, IIIa″, or IIIb″:

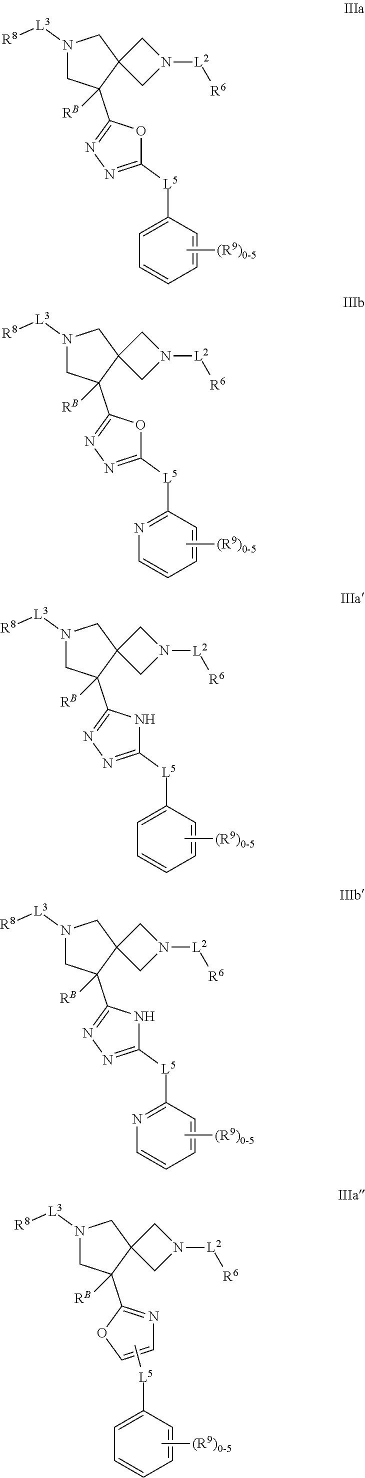

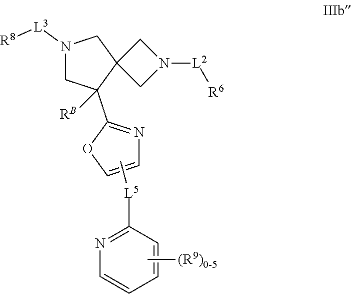

or a pharmaceutically acceptable salt thereof, wherein R^{B}, L², R⁶, R⁹, L³, L⁵, R⁸, R⁹ and their constituent groups, are each as defined and described herein. In some embodiments, L² is a methylene. In some embodiments, L³ is a methylene. In some embodiments, both L² and L³ are methylenes. In some embodiments, -L²-R⁶ is a substituent from Table 3 or Table 4. In some embodiments, -L³-R⁸ is a substituent from Table 5 or Table 6.

In some embodiments, the compound of Formula I is a compound of Formula IIIc, IIId, IIIe, IIIf, IIIg, IIIh, IIIi, IIIj, IIIk, IIIl, IIIm, IIIn, IIIo, IIIp, IIIq, IIIr, IIIs, IIIt, IIIu, IIIv, IIIw, IIIx, IIIy, or IIIz:

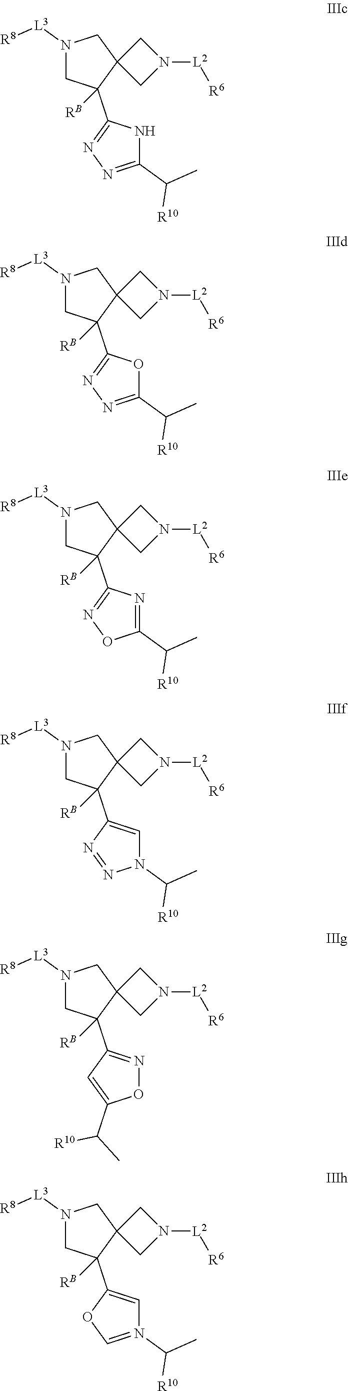

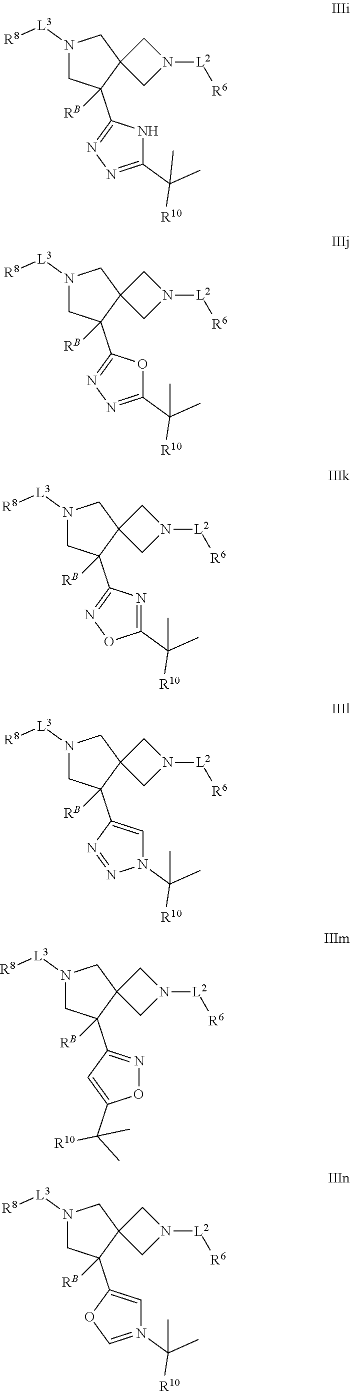

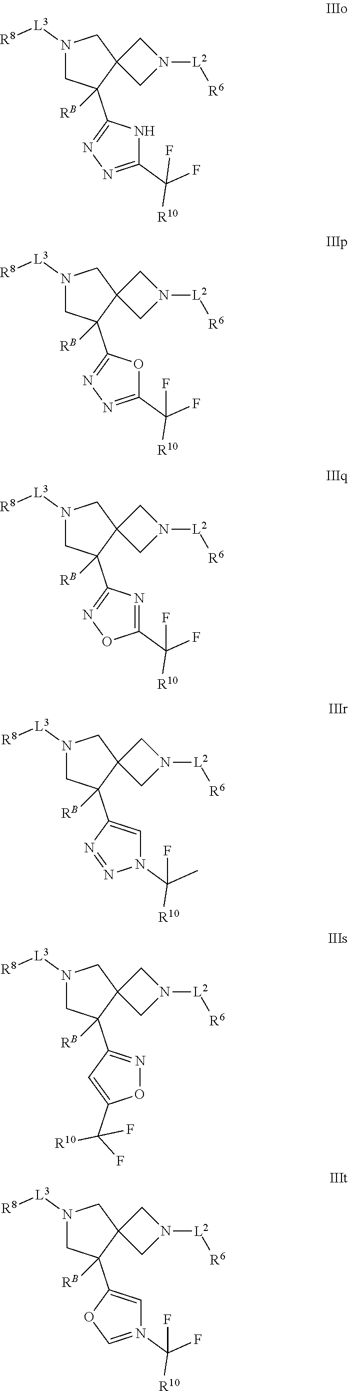

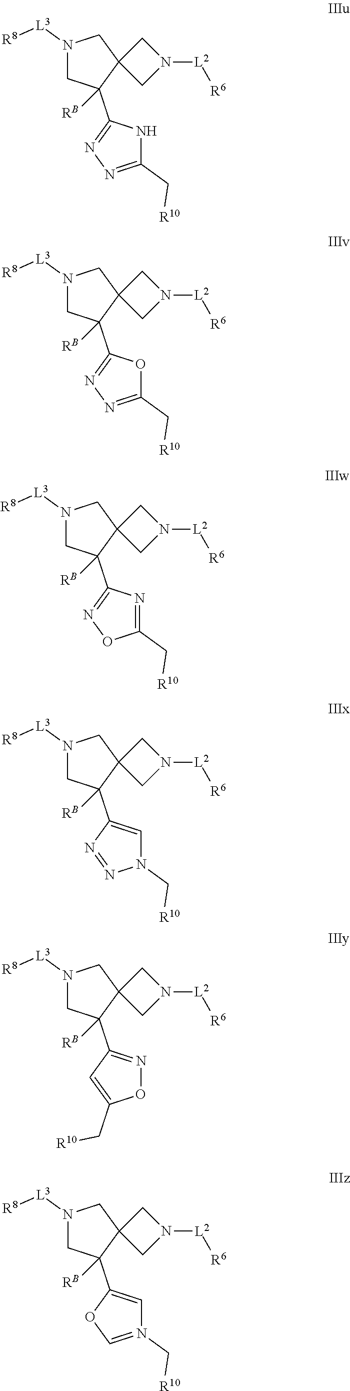

or a pharmaceutically acceptable salt thereof, wherein L², R⁶, L³, R⁸, R^{B}, R¹⁰, and their constituent groups, are each as defined and described herein. In some embodiments, L² is a methylene. In some embodiments, L³ is a methylene. In some embodiments, both L² and L³ are methylenes. In some embodiments, -L²-R⁶ is a substituent from Table 3 or Table 4. In some embodiments, -L³-R⁸ is a substituent from Table 5 or Table 6.

In some embodiments, the compound of Formula I is a compound of Formula IVa:

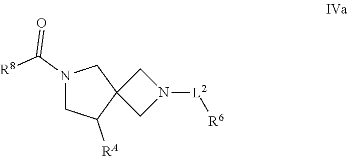

or a pharmaceutically acceptable salt thereof, wherein R^{A}, L², R⁶, and R⁸, and their constituent groups, are each as defined and described herein. In some embodiments, R^{A} is a substituent from Table 1. In some embodiments, -L²-R⁶ is a substituent from Table 3 or Table 4.

In some embodiments, the compound of Formula I is a compound of Formula IVb:

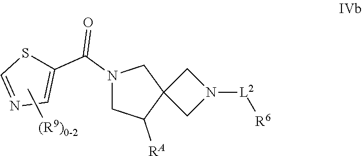

or a pharmaceutically acceptable salt thereof, wherein R^{A}, L², R⁶, and R⁹, and their constituent groups, are each as defined and described herein. In some embodiments, the thiazolyl group is not substituted with R⁹. In some embodiments, R^{A} is a substituent from Table 1. In some embodiments, -L²-R⁶ is a substituent from Table 3 or Table 4.

In some embodiments, the compound of Formula I is a compound of Formula IVc:

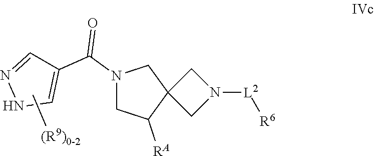

or a pharmaceutically acceptable salt thereof, wherein R^{A}, L², R⁶, and R⁹, and their constituent groups, are each as defined and described herein. In some embodiments, the pyrazolyl group is not substituted with R⁹. In some embodiments, the pyrazolyl group is substituted with one instance of R⁹, which is a benzyl group. In some embodiments, R^{A} is a substituent from Table 1. In some embodiments, -L²-R⁶ is a substituent from Table 3 or Table 4.

In some embodiments, the compound of Formula I is a compound of Formula Va:

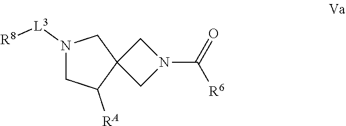

or a pharmaceutically acceptable salt thereof, wherein R^{A}, R⁶, L³ and R⁸, and their constituent groups, are each as defined and described herein. In some embodiments, R⁶ is an optionally substituted cyclopropyl group. In some embodiments, R^{A} is a substituent from Table 1. In some embodiments, -L³-R⁸ is a substituent from Table 5 or Table 6.

In some embodiments, the compound of Formula I is a compound of Formula Vb:

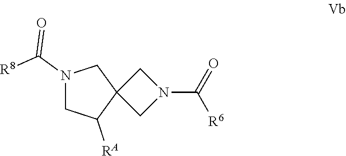

or a pharmaceutically acceptable salt thereof, wherein R^{A}, R⁶, and R⁸, and their constituent groups, are each as defined and described herein. In some embodiments, R⁶ is an optionally substituted cyclopropyl group. In some embodiments, R^{A} is a substituent from Table 1.

In some embodiments, the compound of Formula I is a compound of Formula VIa, VIb, VIc, VId, VIe, VIf, VIg, VIh, VIi, VIj, VIk, VIl, VIm, VIn, VIo, VIp, VIq, VIr, VIs, VIt, VIu, VIv, VIw, VIx, VIy, VIz, VIaa, VIbb, VIcc, or VIdd:

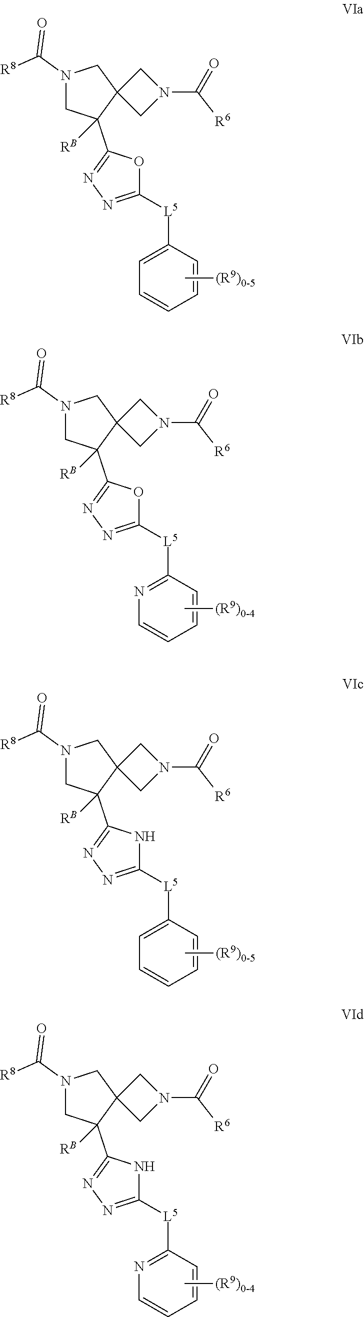

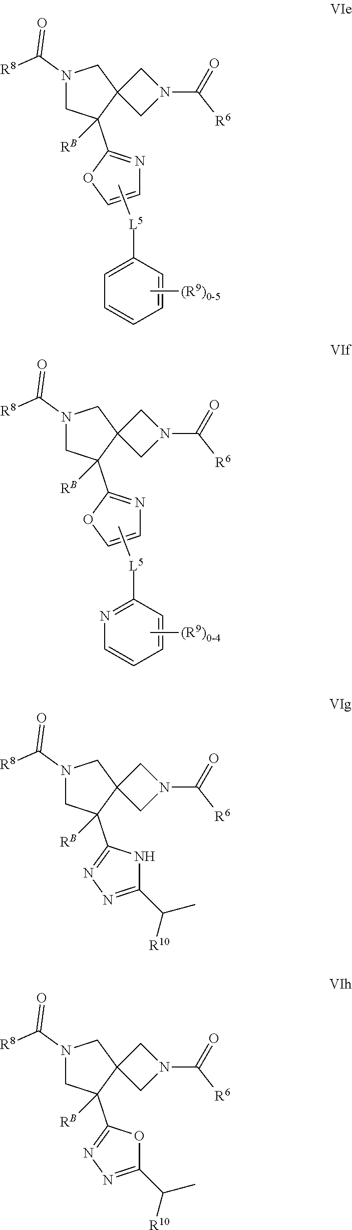

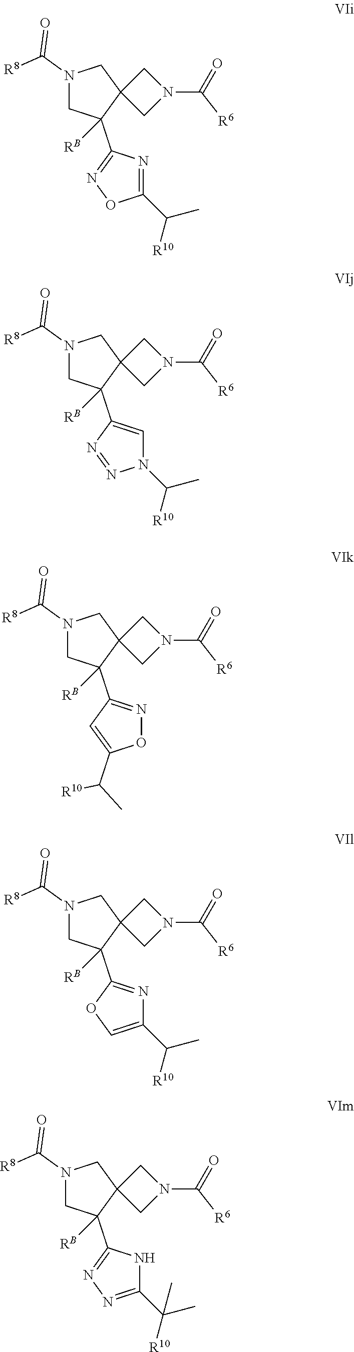

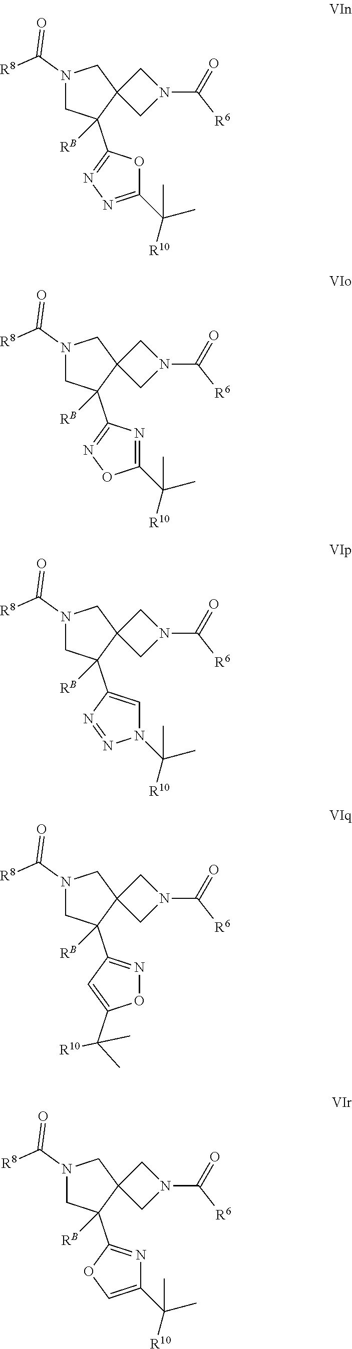

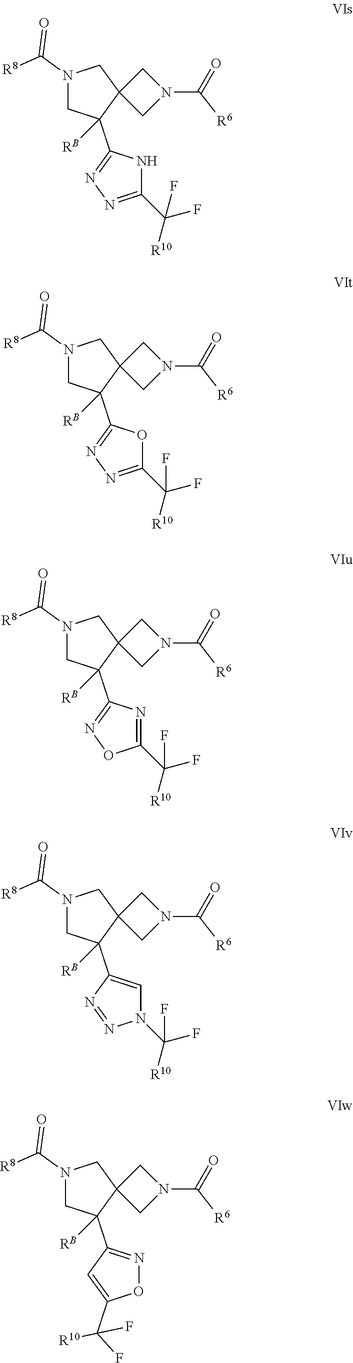

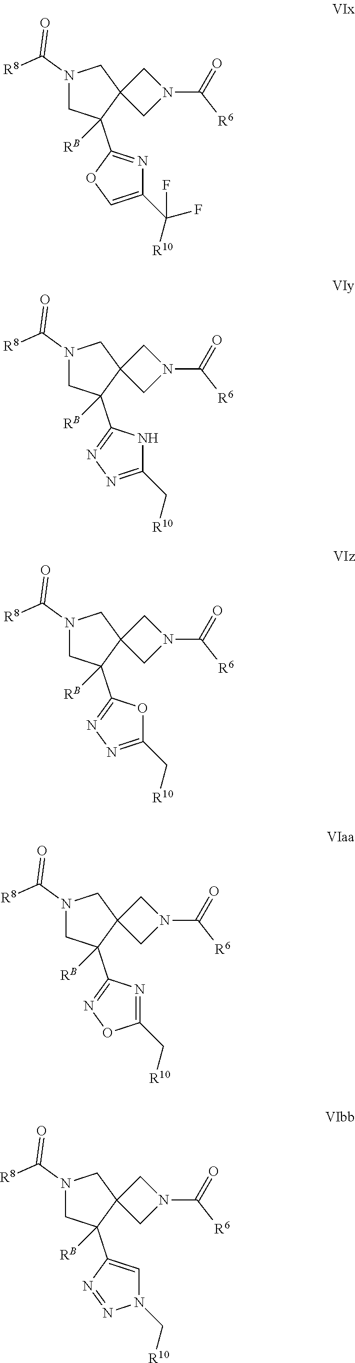

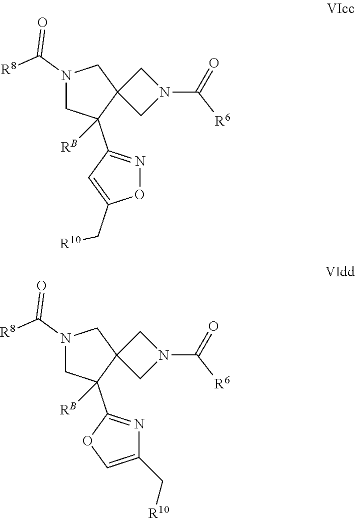

or a pharmaceutically acceptable salt thereof, wherein L⁵, R^{B}, R⁶, R⁸, R⁹, and R¹⁰, and their constituent groups, are each as defined and described herein. In some embodiments, R⁶ is an optionally substituted cyclopropyl group.

In some embodiments, the compound of Formula I is a compound of Formula VIIa, VIIb, VIIc, VIId, VIIe, VIIf, VIIg, VIIh, VIIi, VIIj, VIIk, VIIl, VIIIm, VIIn, VIIo, VIIp, VIIq, VIIr, VIIs, VIIt, VIIu, VIIv, VIIw, VIIx, VIIy, VIIz, VIIaa, VIIbb, VIIcc, or VIIdd:

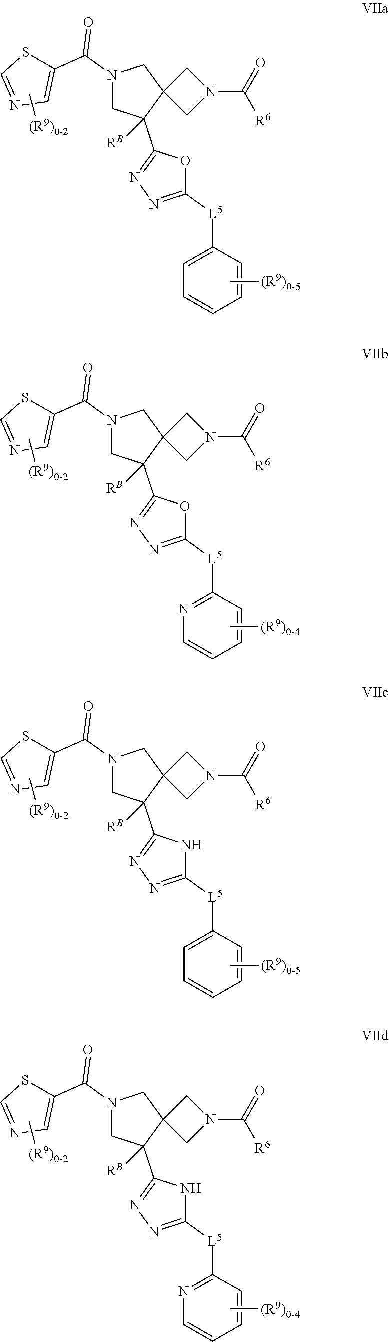

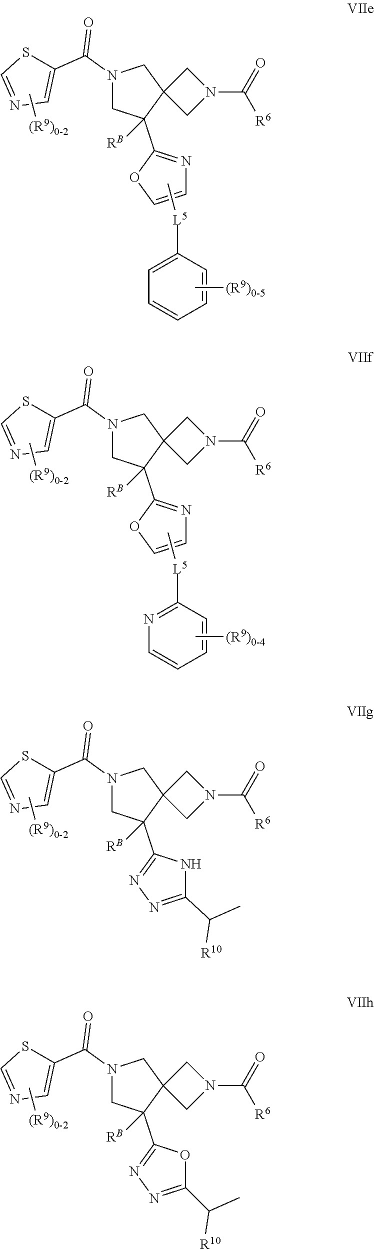

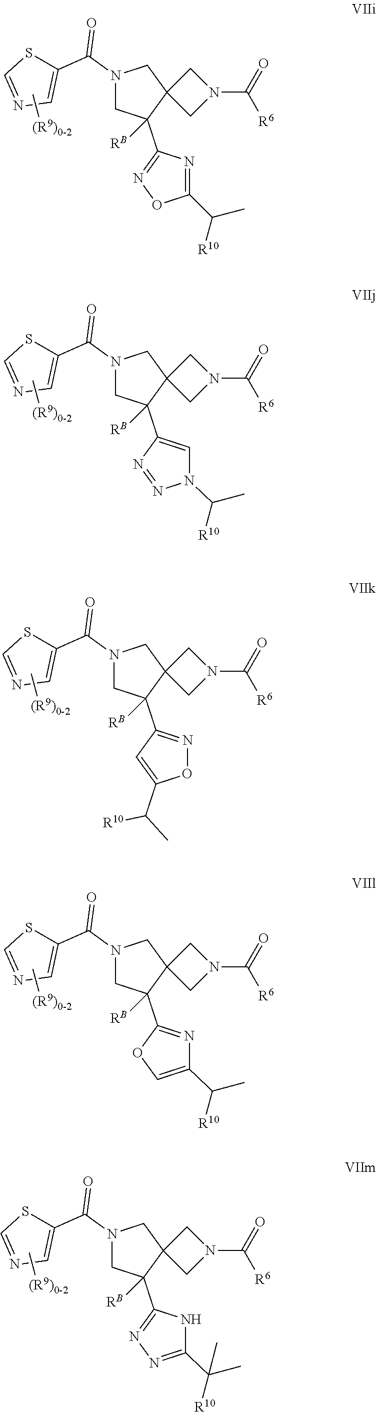

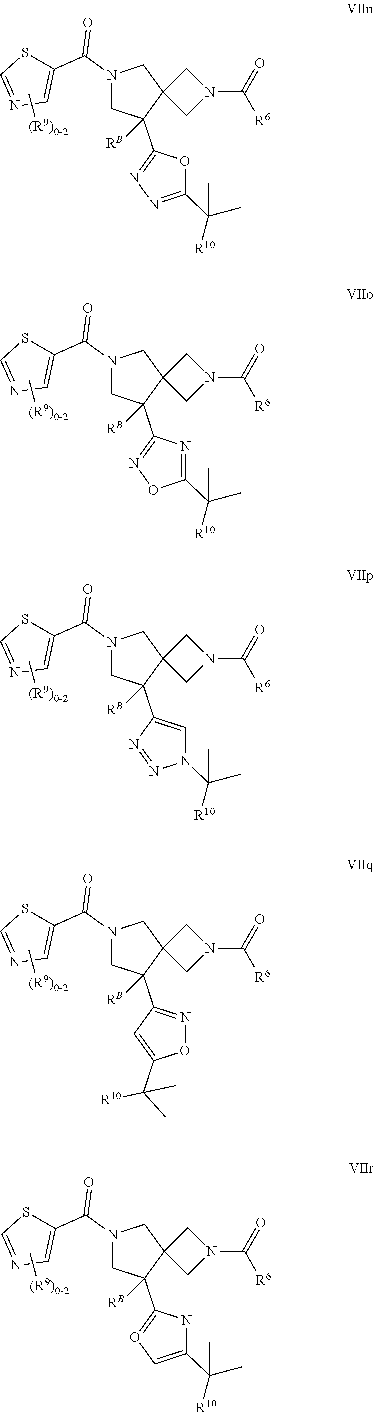

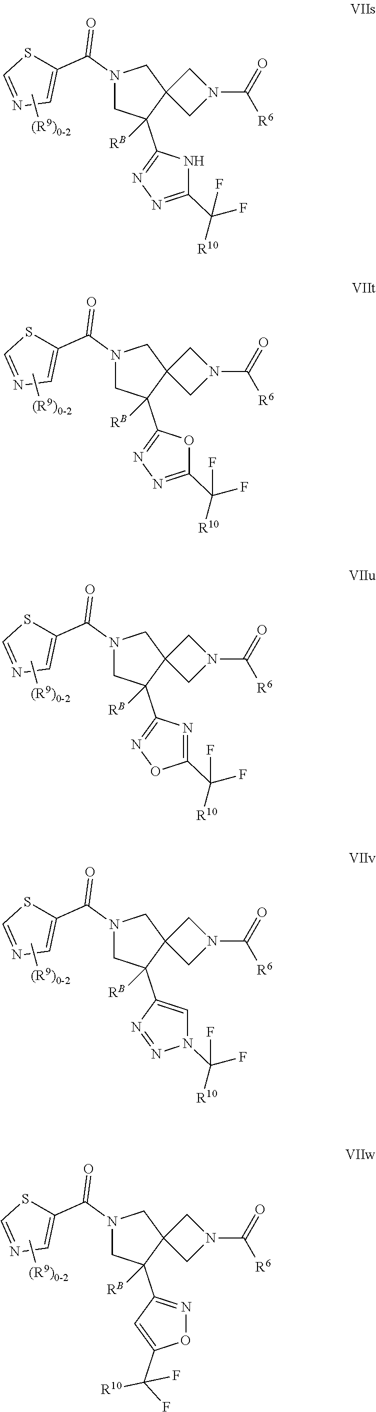

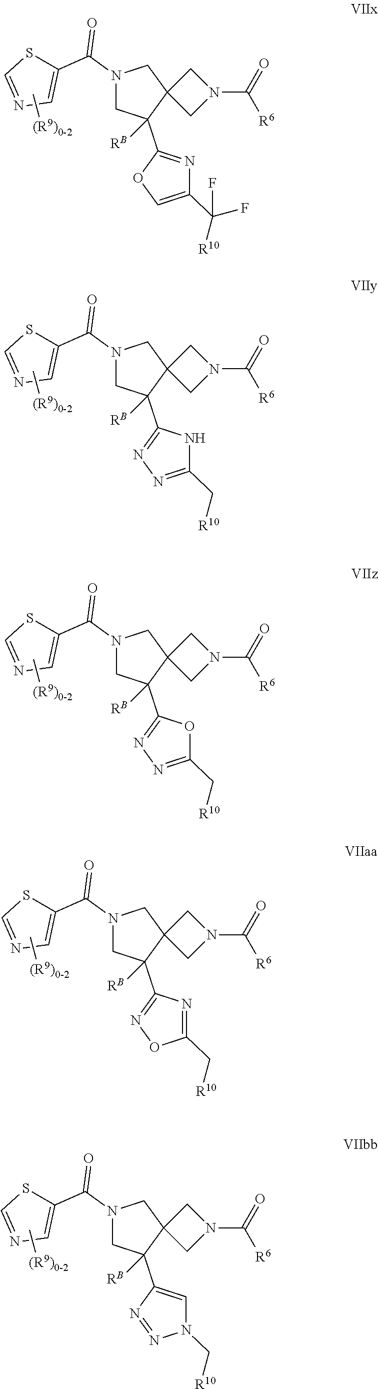

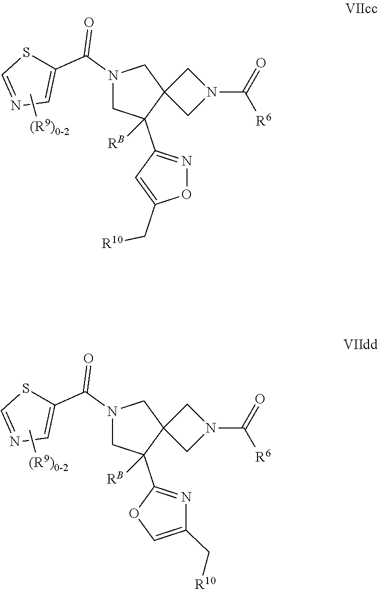

or a pharmaceutically acceptable salt thereof, wherein L⁵, R^{B}, R⁶, independently each R⁹, and R¹⁰, and their constituent groups, are each as defined and described herein. In some embodiments, R⁶ is an optionally substituted cyclopropyl group. In some embodiments, the thiazolyl group is not substituted with R⁹.

In some embodiments, the compound of Formula I is a compound of Formula VIIIa, VIIIb VIIIc, VIIId, VIIIe, VIIIf, VIIIg, VIIIh, VIIIi, VIIIj, VIIIk, VIIIl, VIIIm, VIIIn, VIIIo, VIIIp, VIIIq, VIIIr, VIIIs, VIIIt, VIIIu, VIIIv, VIIIw, VIIIx, VIIIy, VIIIz, VIIIaa, VIIIbb, VIIIcc, or VIIIdd:

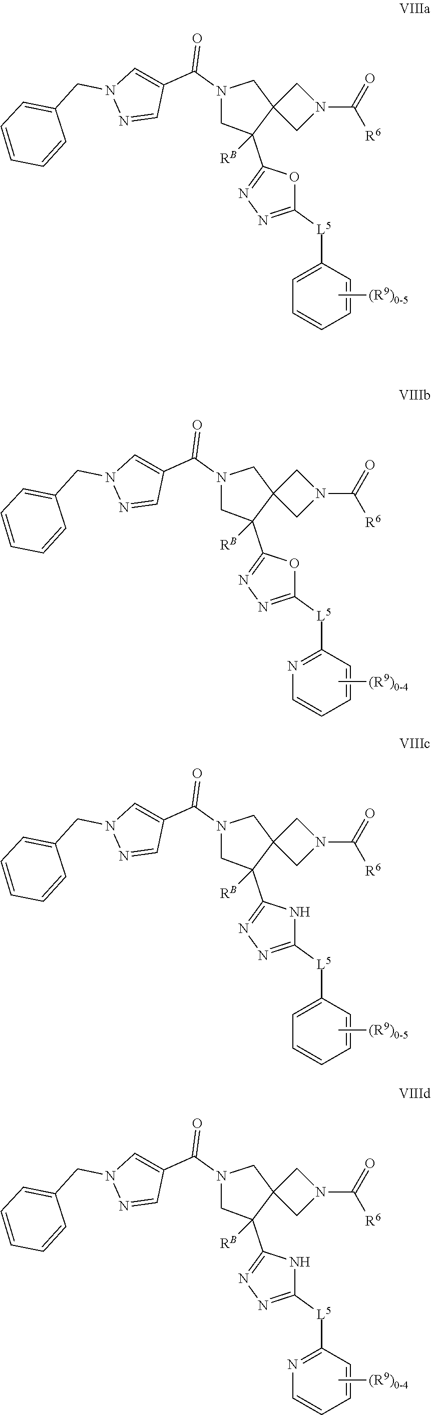

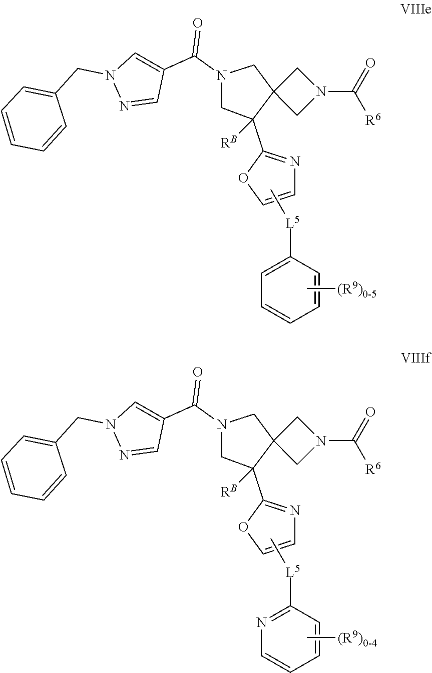

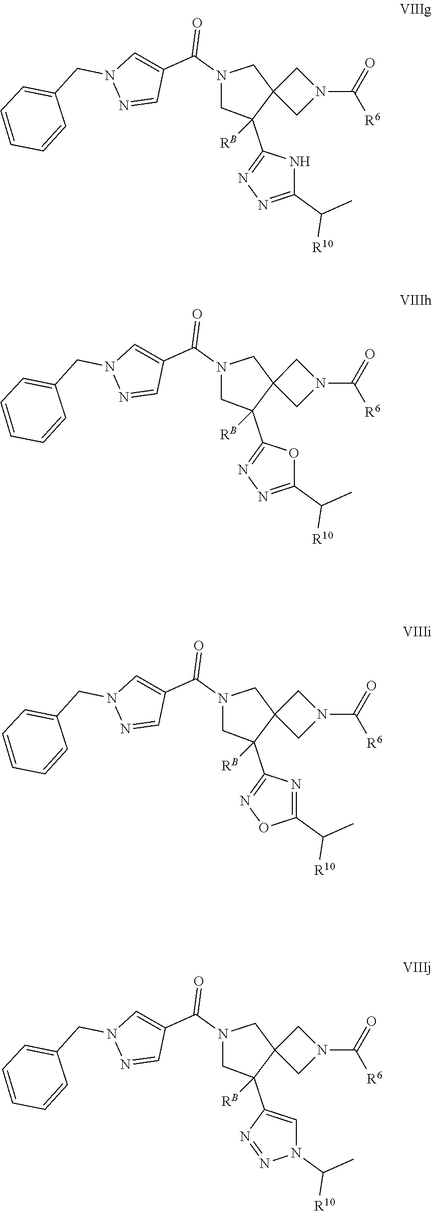

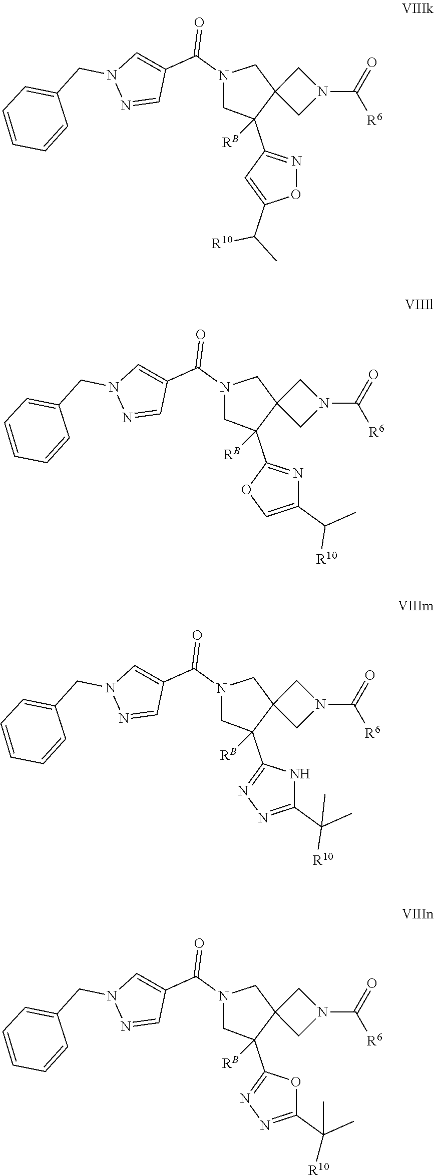

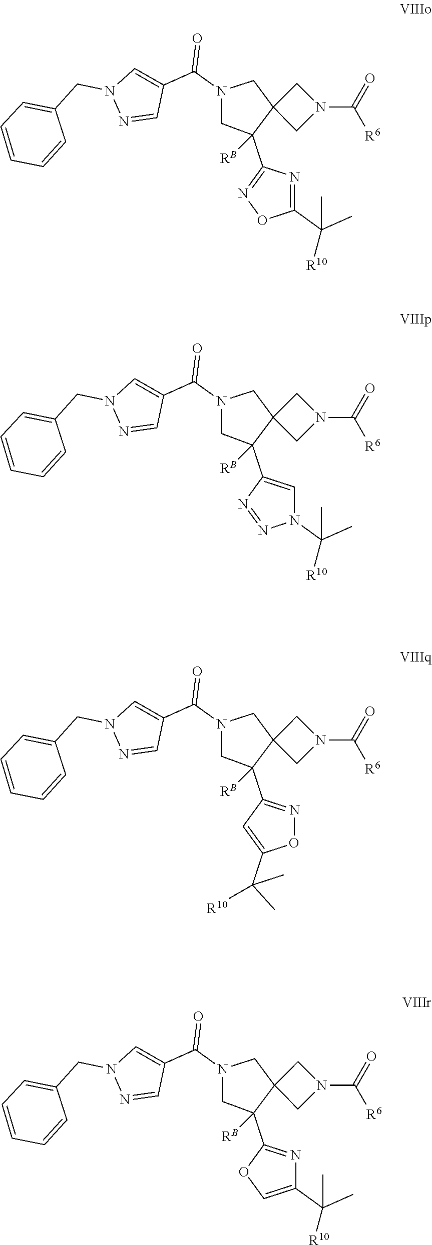

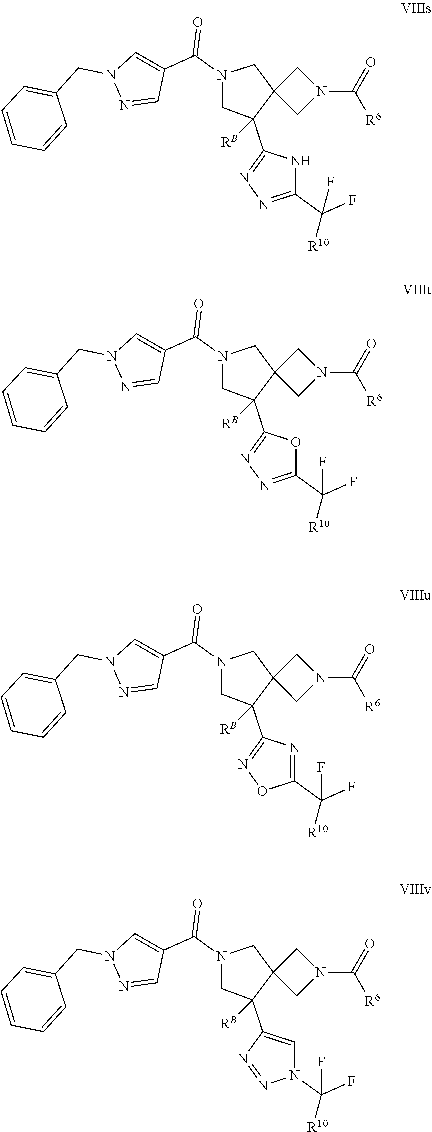

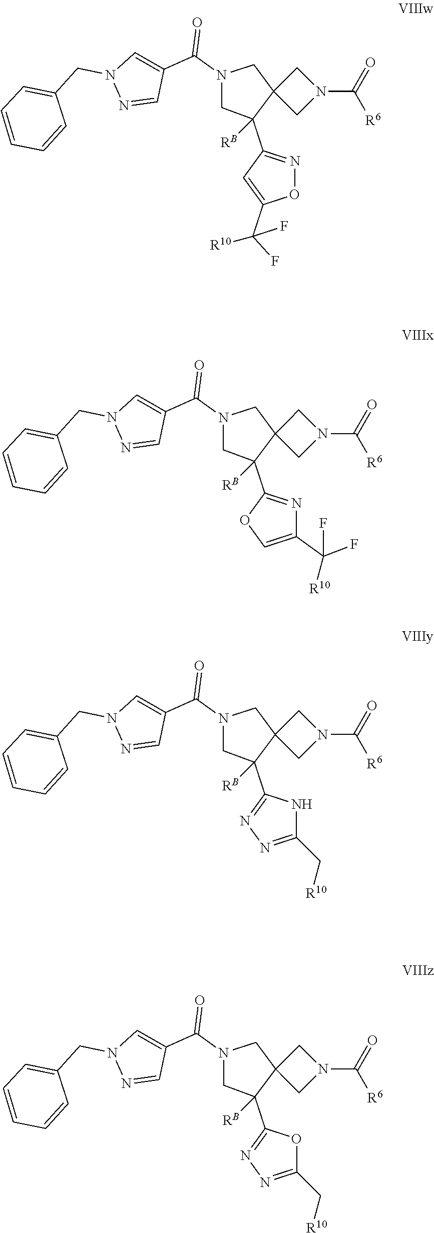

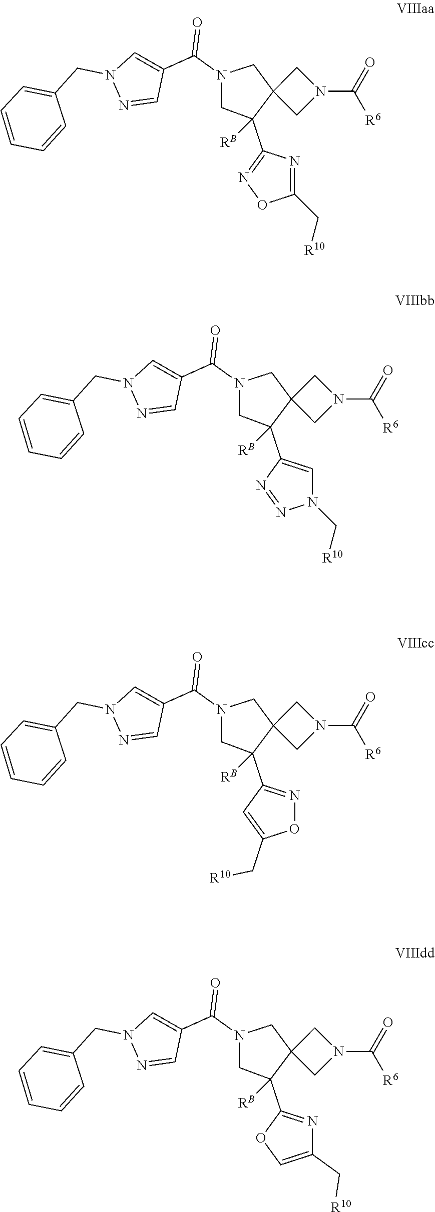

or a pharmaceutically acceptable salt thereof, wherein L⁵, R^{B}, R⁶, R¹⁰, and their constituent groups, are each as defined and described herein. In some embodiments, R⁶ is an optionally substituted cyclopropyl group.

In some embodiments, the compound of Formula I is a compound of Formula IXa, IXb, IXc, IXd, IXe, IXf, IXg, IXh, IXi, IXj, IXk, IXl, IXm, IXn, IXo, IXp, IXq, IXr, IXs, IXt, IXu, IXv, IXw, IXx, IXy, IXz, IXaa, IXbb, IXcc, or IXdd:

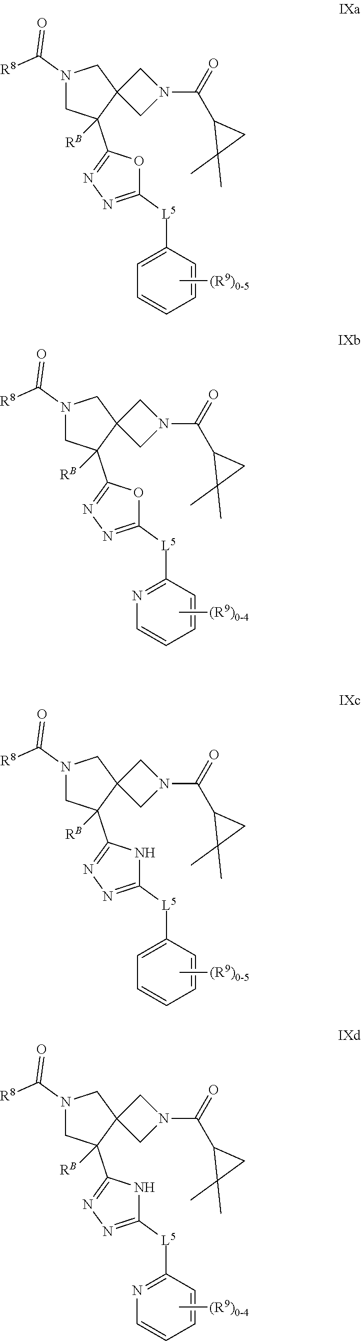

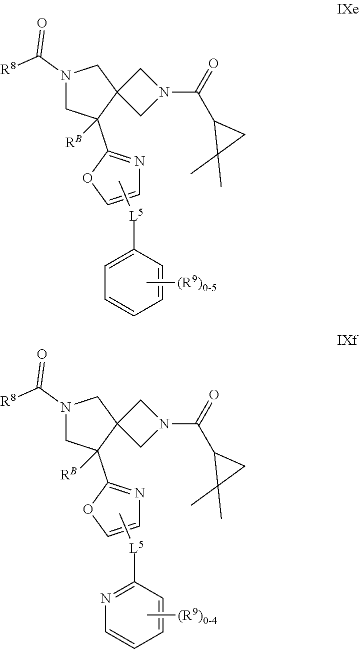

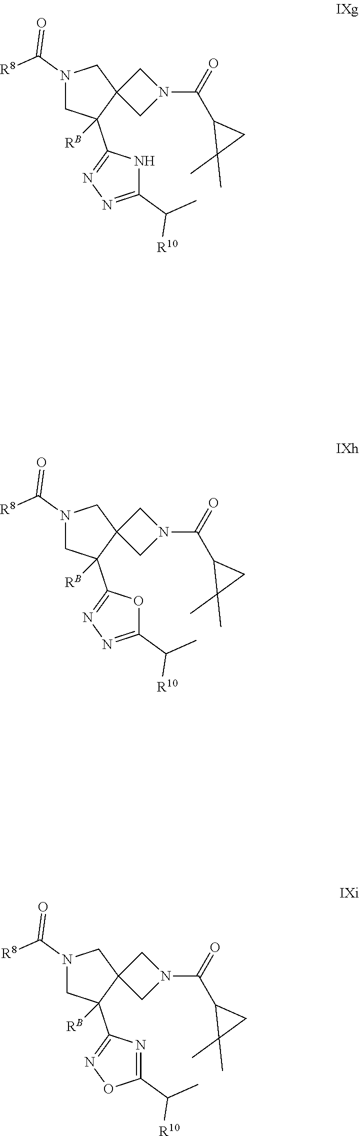

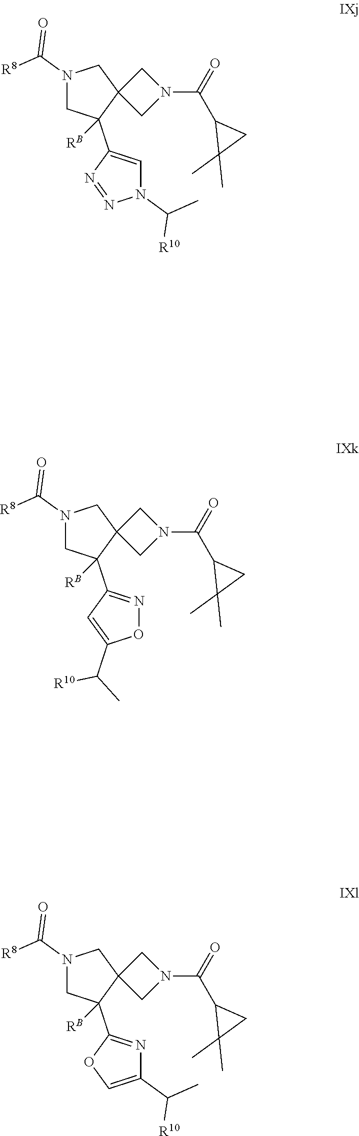

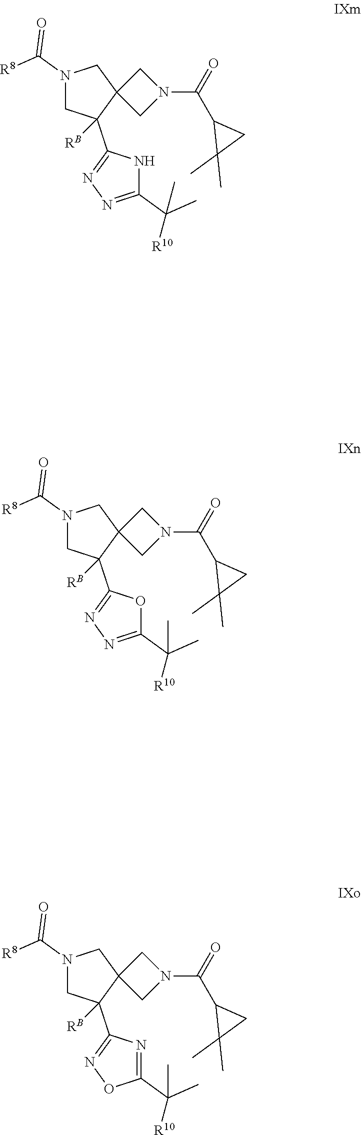

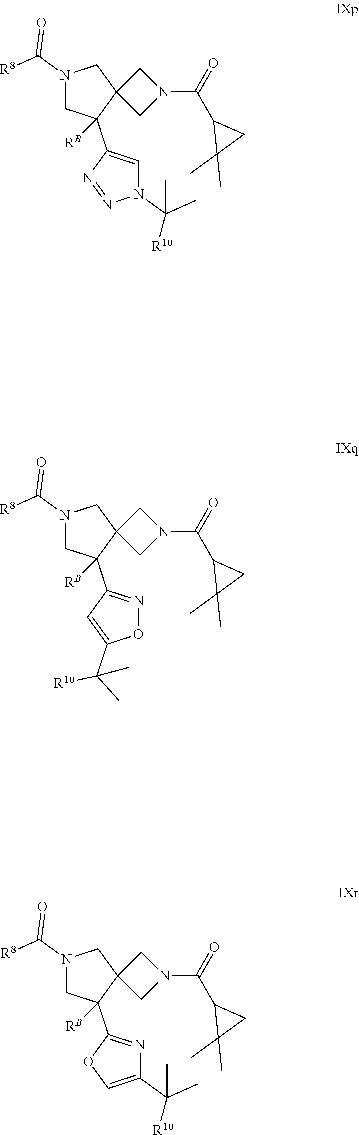

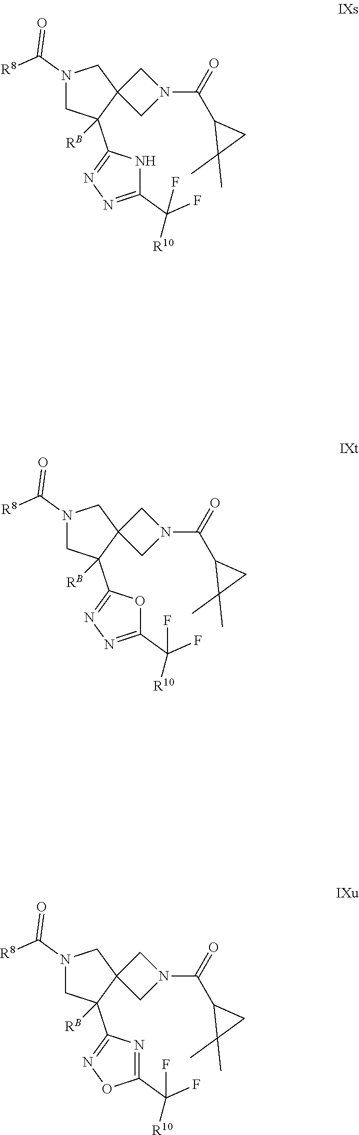

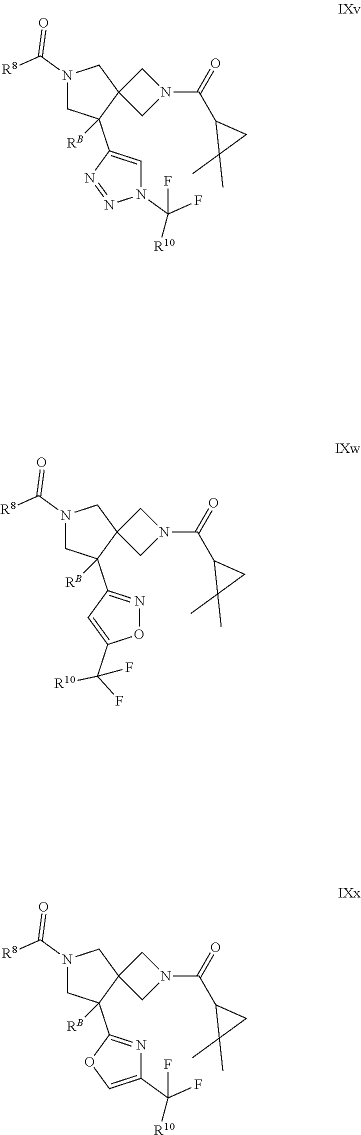

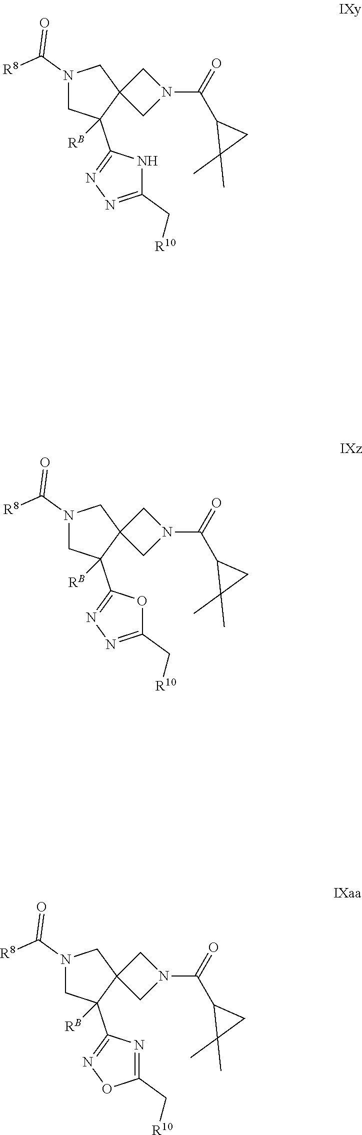

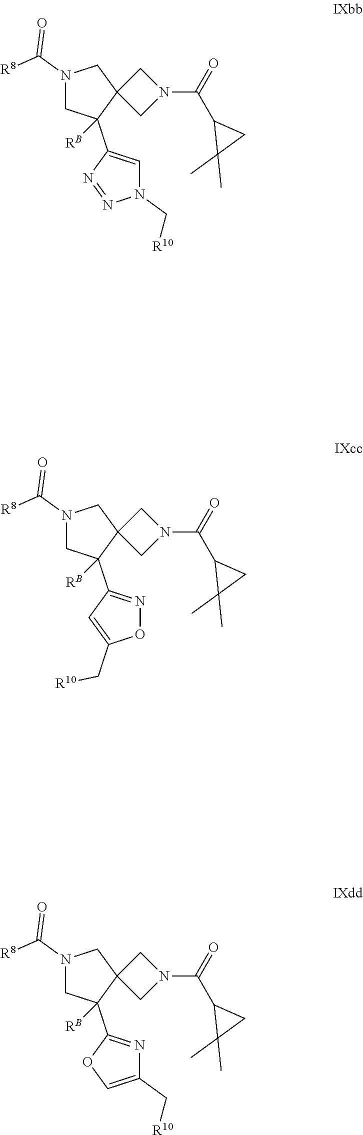

or a pharmaceutically acceptable salt thereof, wherein L⁵, R^{B}, R⁸, and R¹⁰, and their constituent groups, are each as defined and described herein. In some embodiments, R⁸ is an optionally substituted 5-6 membered heteroaryl group.

In some embodiments, the compound of Formula I is a compound of Formula Xa, Xb, Xc, Xd, Xe, Xf, Xg, Xh, Xi, Xj, Xk, or Xl:

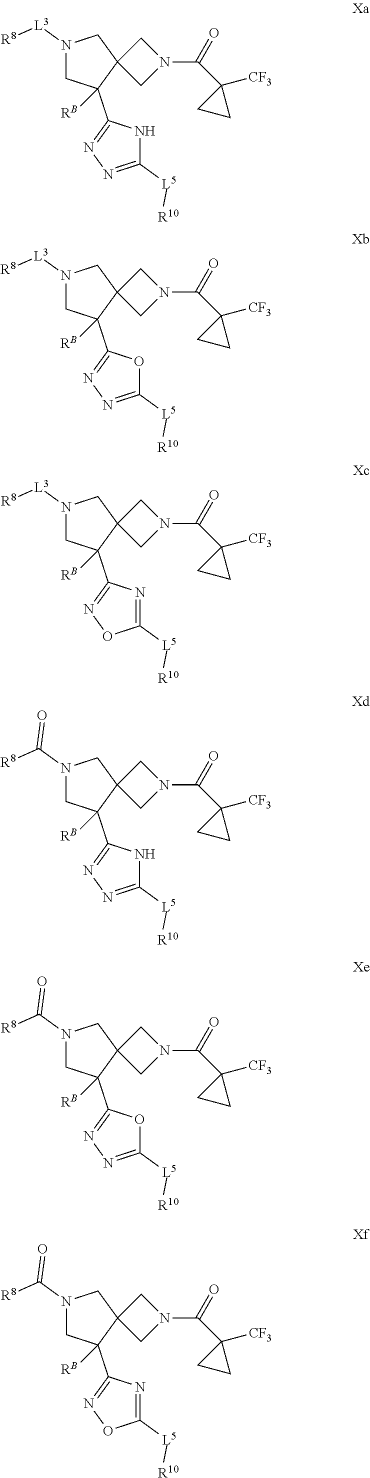

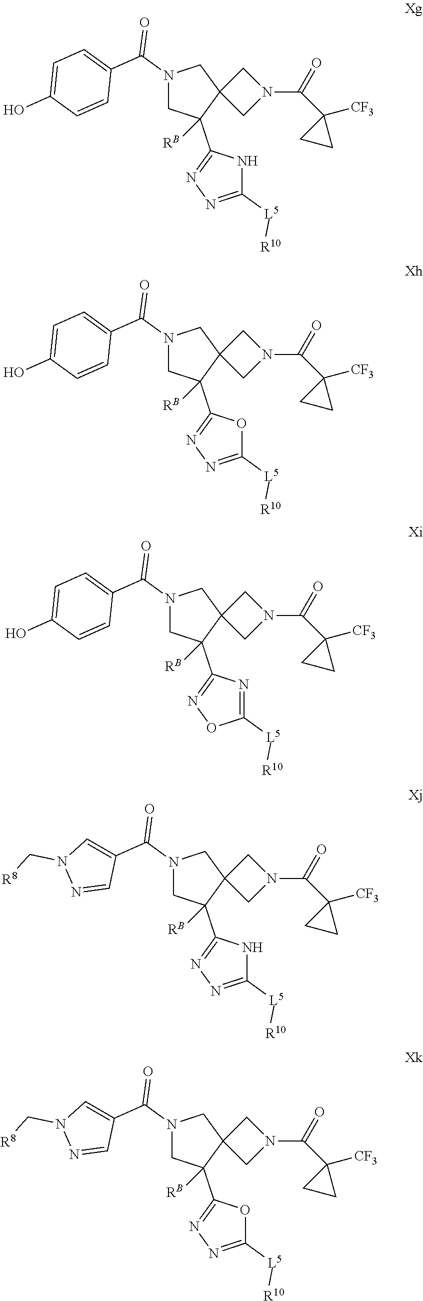

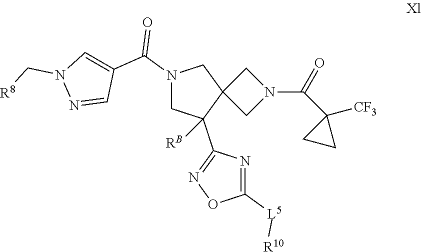

or a pharmaceutically acceptable salt thereof, wherein, when present, L³, L⁵, R^{B}, R⁸, and R¹⁰, and their constituent groups, are each as defined and described herein. In some embodiments, the compound of Formula I is a compound of Formula Xa, Xb, Xc, Xd, Xe, Xf, Xg, Xh, Xi, Xj, Xk, or Xl, wherein -L⁵-R¹⁰ is selected from

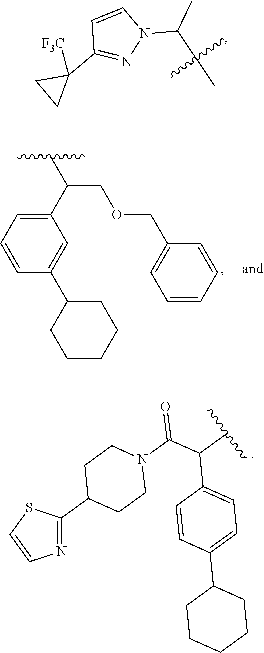 [SMILES: *C(C)n1ccc(C2(C(F)(F)F)CC2)n1 ; *C(COCc1ccccc1)c1cccc(C2CCCCC2)c1 ; *C(C(=O)N1CCC(c2nccs2)CC1)c1ccc(C2CCCCC2)cc1]

Exemplary compounds of the present disclosure are set forth in Table 8, below.

TABLE 8
Exemplary Compounds

| Compound | |
|---|---|
| # | Structure |
| I-1 | 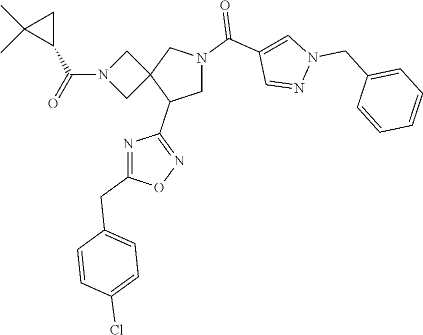 [SMILES: CC1(C)C[C@@H]1C(=O)N1CC2(CN(C(=O)c3cnn(Cc4ccccc4)c3)CC2c2noc(Cc3ccc(Cl)cc3)n2)C1] |
| I-2 | 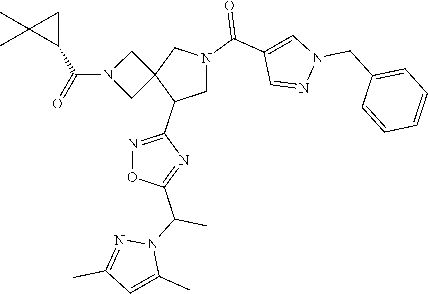 [SMILES: Cc1cc(C)n(C(C)c2nc(C3CN(C(=O)c4cnn(Cc5ccccc5)c4)CC34CN(C(=O)[C@H]3CC3(C)C)C4)no2)n1] |
| | (A) |
| | 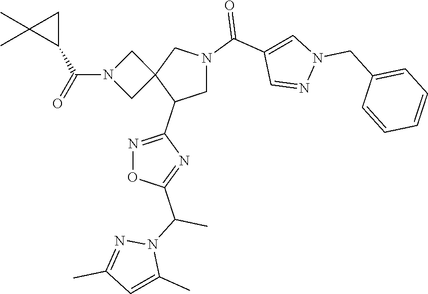 [SMILES: Cc1cc(C)n(C(C)c2nc(C3CN(C(=O)c4cnn(Cc5ccccc5)c4)CC34CN(C(=O)[C@H]3CC3(C)C)C4)no2)n1] |
| | (B) |
| | 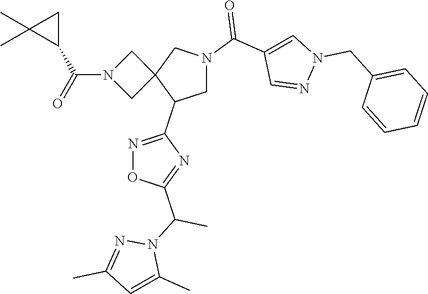 [SMILES: Cc1cc(C)n(C(C)c2nc(C3CN(C(=O)c4cnn(Cc5ccccc5)c4)CC34CN(C(=O)[C@H]3CC3(C)C)C4)no2)n1] |
| | (C) |
| | 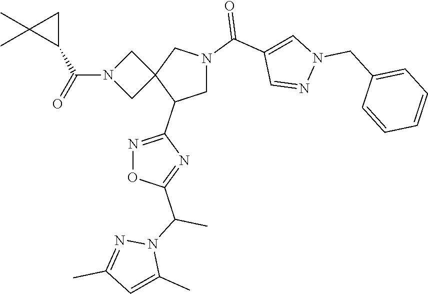 [SMILES: Cc1cc(C)n(C(C)c2nc(C3CN(C(=O)c4cnn(Cc5ccccc5)c4)CC34CN(C(=O)[C@H]3CC3(C)C)C4)no2)n1] |
| | (D) |
| I-3 | 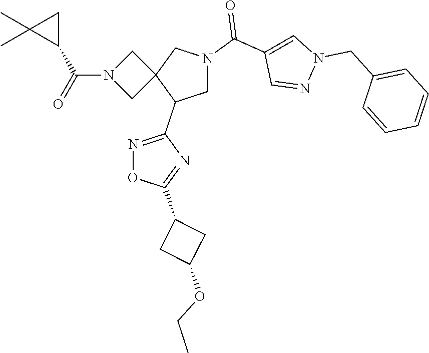 [SMILES: CCO[C@H]1C[C@@H](c2nc(C3CN(C(=O)c4cnn(Cc5ccccc5)c4)CC34CN(C(=O)[C@H]3CC3(C)C)C4)no2)C1] |
| I-7 | 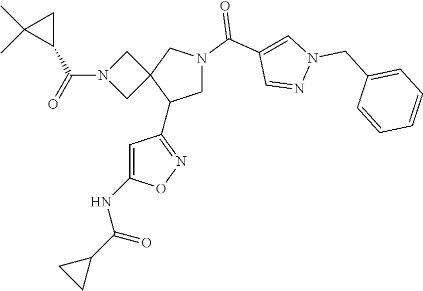 [SMILES: CC1(C)C[C@@H]1C(=O)N1CC2(CN(C(=O)c3cnn(Cc4ccccc4)c3)CC2c2cc(NC(=O)C3CC3)on2)C1] |
| I-5 | 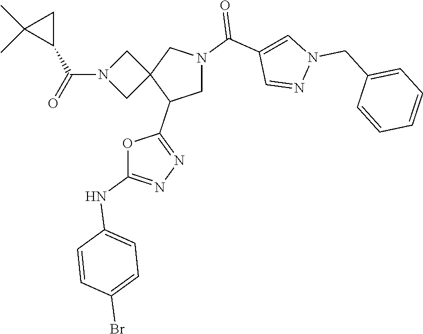 [SMILES: CC1(C)C[C@@H]1C(=O)N1CC2(CN(C(=O)c3cnn(Cc4ccccc4)c3)CC2c2nnc(Nc3ccc(Br)cc3)o2)C1] |
| I-6 | 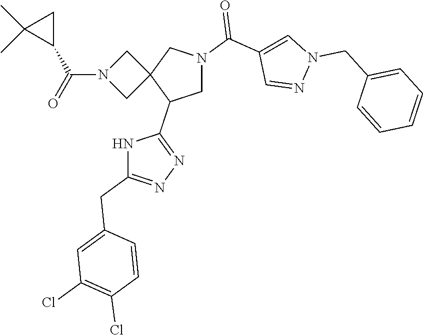 [SMILES: CC1(C)C[C@@H]1C(=O)N1CC2(CN(C(=O)c3cnn(Cc4ccccc4)c3)CC2c2nnc(Cc3ccc(Cl)c(Cl)c3)[nH]2)C1] |
| I-4 | 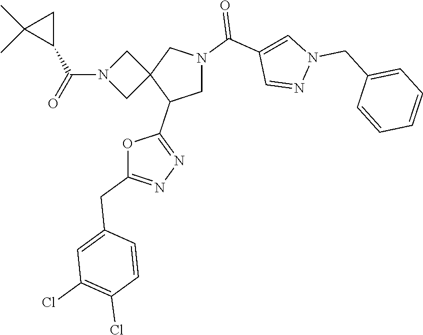 [SMILES: CC1(C)C[C@@H]1C(=O)N1CC2(CN(C(=O)c3cnn(Cc4ccccc4)c3)CC2c2nnc(Cc3ccc(Cl)c(Cl)c3)o2)C1] |
| I-8 | 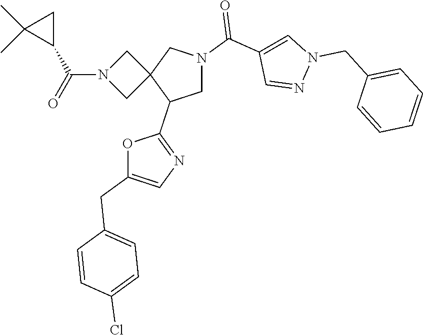 [SMILES: CC1(C)C[C@@H]1C(=O)N1CC2(CN(C(=O)c3cnn(Cc4ccccc4)c3)CC2c2ncc(Cc3ccc(Cl)cc3)o2)C1] |
| I-9 | 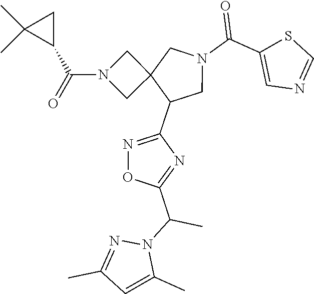 [SMILES: Cc1cc(C)n(C(C)c2nc(C3CN(C(=O)c4cncs4)CC34CN(C(=O)[C@H]3CC3(C)C)C4)no2)n1] |
| I-10 | 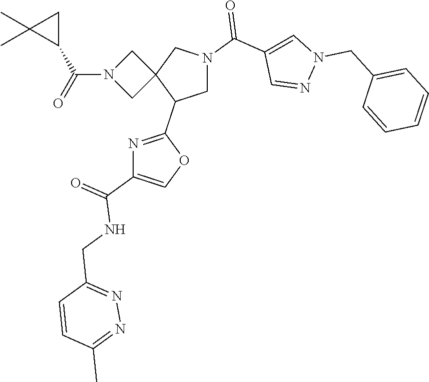 [SMILES: Cc1ccc(CNC(=O)c2coc(C3CN(C(=O)c4cnn(Cc5ccccc5)c4)CC34CN(C(=O)[C@H]3CC3(C)C)C4)n2)nn1] |
| I-11 | 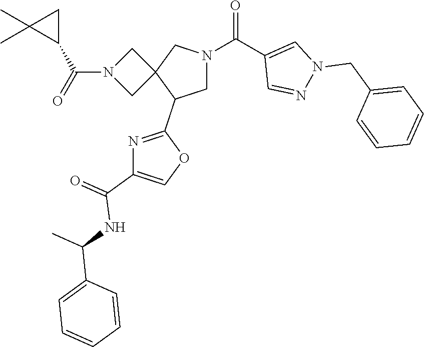 [SMILES: C[C@@H](NC(=O)c1coc(C2CN(C(=O)c3cnn(Cc4ccccc4)c3)CC23CN(C(=O)[C@H]2CC2(C)C)C3)n1)c1ccccc1] |
| I-12 | 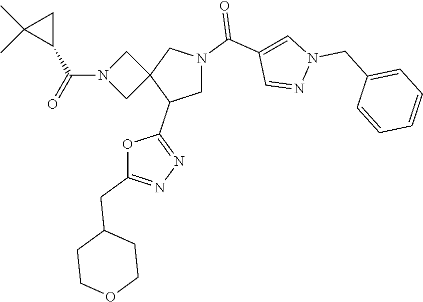 [SMILES: CC1(C)C[C@@H]1C(=O)N1CC2(CN(C(=O)c3cnn(Cc4ccccc4)c3)CC2c2nnc(CC3CCOCC3)o2)C1] |
| I-13 | 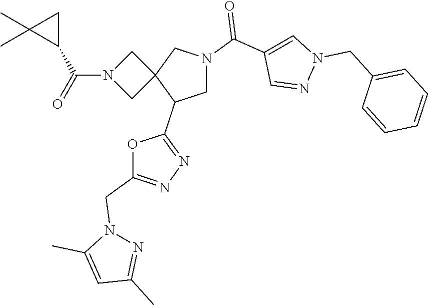 [SMILES: Cc1cc(C)n(Cc2nnc(C3CN(C(=O)c4cnn(Cc5ccccc5)c4)CC34CN(C(=O)[C@H]3CC3(C)C)C4)o2)n1] |
| I-14 | 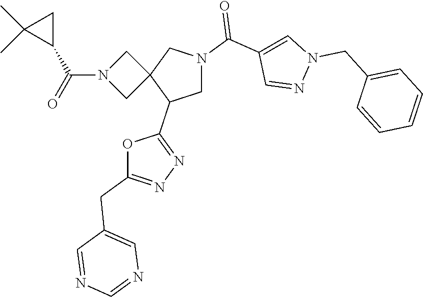 [SMILES: CC1(C)C[C@@H]1C(=O)N1CC2(CN(C(=O)c3cnn(Cc4ccccc4)c3)CC2c2nnc(Cc3cncnc3)o2)C1] |
| I-15 | 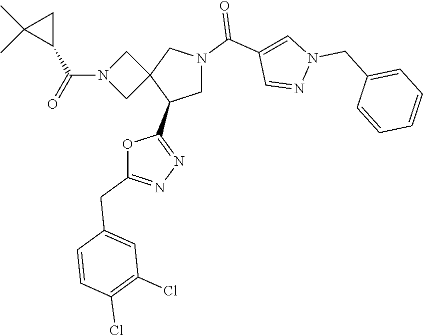 [SMILES: CC1(C)C[C@@H]1C(=O)N1CC2(CN(C(=O)c3cnn(Cc4ccccc4)c3)C[C@H]2c2nnc(Cc3ccc(Cl)c(Cl)c3)o2)C1] |
| I-16 | 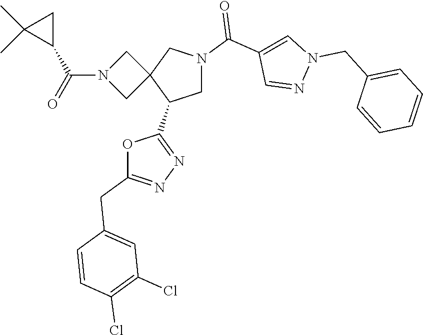 [SMILES: CC1(C)C[C@@H]1C(=O)N1CC2(CN(C(=O)c3cnn(Cc4ccccc4)c3)C[C@@H]2c2nnc(Cc3ccc(Cl)c(Cl)c3)o2)C1] |
| I-17 | 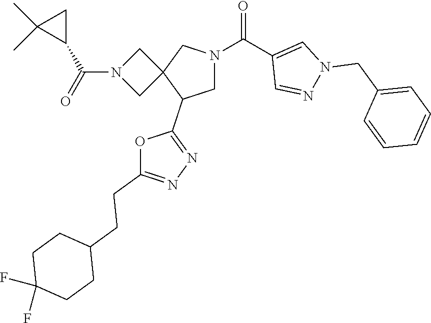 [SMILES: CC1(C)C[C@@H]1C(=O)N1CC2(CN(C(=O)c3cnn(Cc4ccccc4)c3)CC2c2nnc(CCC3CCC(F)(F)CC3)o2)C1] |
| I-18 | 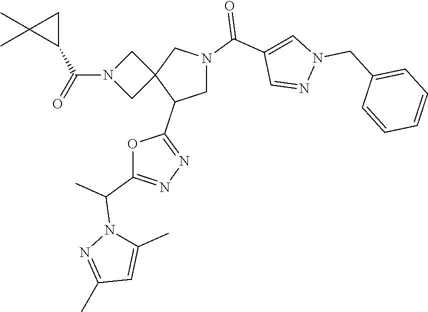 [SMILES: Cc1cc(C)n(C(C)c2nnc(C3CN(C(=O)c4cnn(Cc5ccccc5)c4)CC34CN(C(=O)[C@H]3CC3(C)C)C4)o2)n1] |
| I-19 | 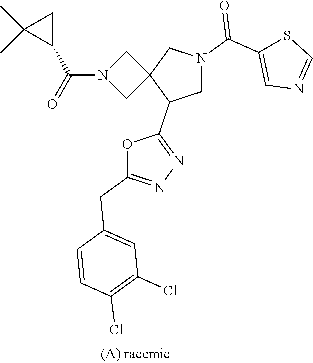 [SMILES: CC1(C)C[C@@H]1C(=O)N1CC2(CN(C(=O)c3cncs3)CC2c2nnc(Cc3ccc(Cl)c(Cl)c3)o2)C1] |
| | 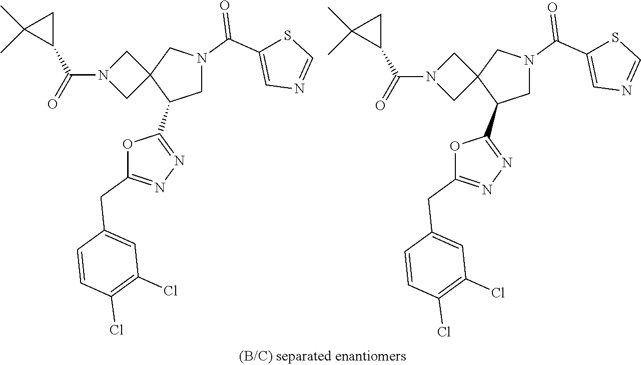 |
| I-20 | 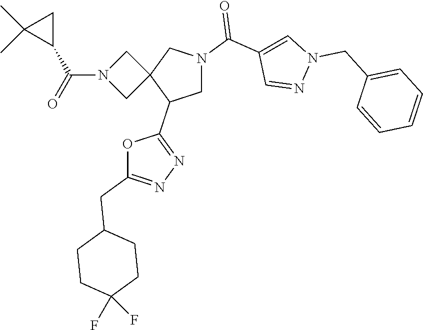 [SMILES: CC1(C)C[C@@H]1C(=O)N1CC2(CN(C(=O)c3cnn(Cc4ccccc4)c3)CC2c2nnc(CC3CCC(F)(F)CC3)o2)C1] |
| I-21 | 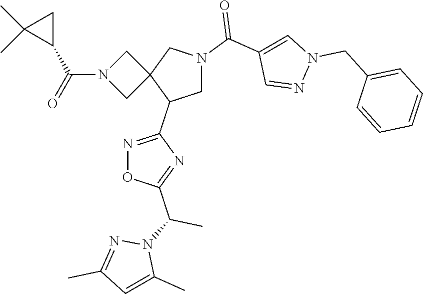 [SMILES: Cc1cc(C)n([C@@H](C)c2nc(C3CN(C(=O)c4cnn(Cc5ccccc5)c4)CC34CN(C(=O)[C@H]3CC3(C)C)C4)no2)n1] |
| I-22 | 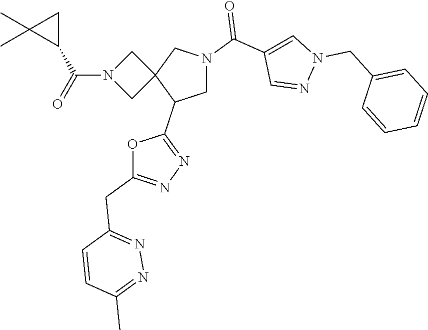 [SMILES: Cc1ccc(Cc2nnc(C3CN(C(=O)c4cnn(Cc5ccccc5)c4)CC34CN(C(=O)[C@H]3CC3(C)C)C4)o2)nn1] |
| I-23 | 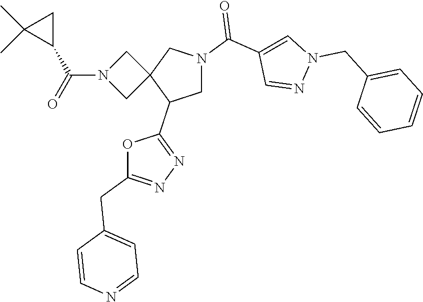 [SMILES: CC1(C)C[C@@H]1C(=O)N1CC2(CN(C(=O)c3cnn(Cc4ccccc4)c3)CC2c2nnc(Cc3ccncc3)o2)C1] |
| I-24 | 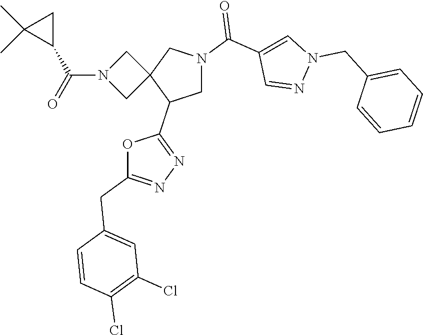 [SMILES: CC1(C)C[C@@H]1C(=O)N1CC2(CN(C(=O)c3cnn(Cc4ccccc4)c3)CC2c2nnc(Cc3ccc(Cl)c(Cl)c3)o2)C1] |
| I-25 | 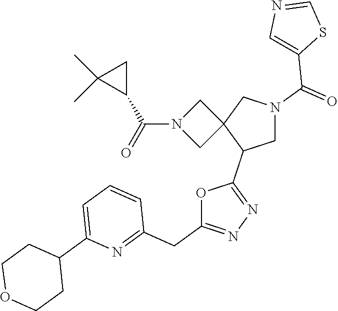 [SMILES: CC1(C)C[C@@H]1C(=O)N1CC2(CN(C(=O)c3cncs3)CC2c2nnc(Cc3cccc(C4CCOCC4)n3)o2)C1] |
| I-26 | 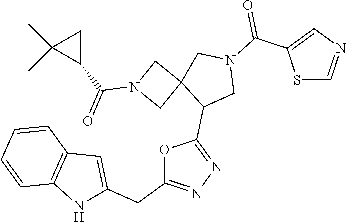 [SMILES: CC1(C)C[C@@H]1C(=O)N1CC2(CN(C(=O)c3cncs3)CC2c2nnc(Cc3cc4ccccc4[nH]3)o2)C1] |
| I-27 | 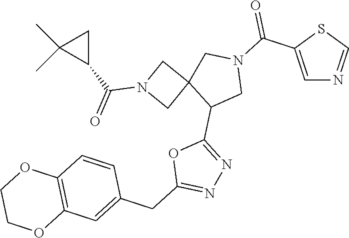 [SMILES: CC1(C)C[C@@H]1C(=O)N1CC2(CN(C(=O)c3cncs3)CC2c2nnc(Cc3ccc4c(c3)OCCO4)o2)C1] |
| I-28 | 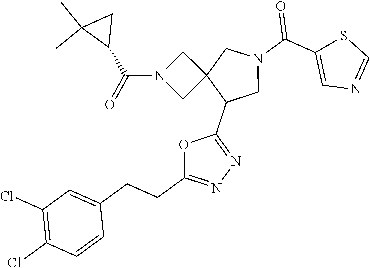 [SMILES: CC1(C)C[C@@H]1C(=O)N1CC2(CN(C(=O)c3cncs3)CC2c2nnc(CCc3ccc(Cl)c(Cl)c3)o2)C1] |
| I-29 | 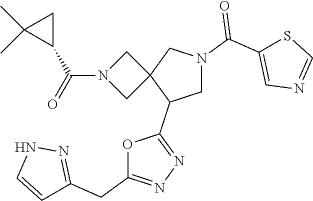 [SMILES: CC1(C)C[C@@H]1C(=O)N1CC2(CN(C(=O)c3cncs3)CC2c2nnc(Cc3cc[nH]n3)o2)C1] |
| I-30 | 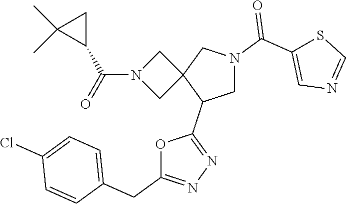 [SMILES: CC1(C)C[C@@H]1C(=O)N1CC2(CN(C(=O)c3cncs3)CC2c2nnc(Cc3ccc(Cl)cc3)o2)C1] |
| I-31 | 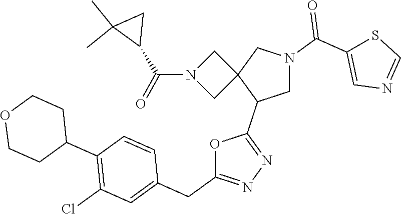 [SMILES: CC1(C)C[C@@H]1C(=O)N1CC2(CN(C(=O)c3cncs3)CC2c2nnc(Cc3ccc(C4CCOCC4)c(Cl)c3)o2)C1] |
| I-32 | 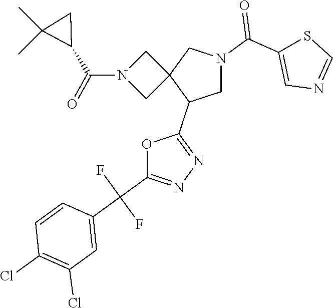 [SMILES: CC1(C)C[C@@H]1C(=O)N1CC2(CN(C(=O)c3cncs3)CC2c2nnc(C(F)(F)c3ccc(Cl)c(Cl)c3)o2)C1] |
| I-33 | 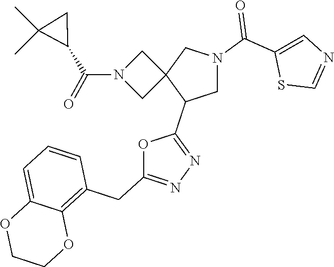 [SMILES: CC1(C)C[C@@H]1C(=O)N1CC2(CN(C(=O)c3cncs3)CC2c2nnc(Cc3cccc4c3OCCO4)o2)C1] |
| I-34 | 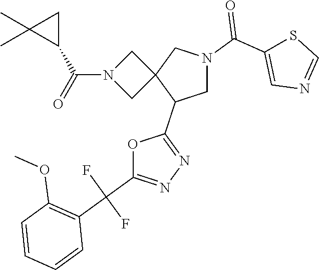 [SMILES: COc1ccccc1C(F)(F)c1nnc(C2CN(C(=O)c3cncs3)CC23CN(C(=O)[C@H]2CC2(C)C)C3)o1] |
| I-35 | 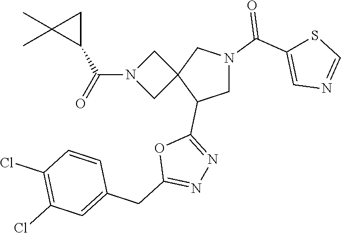 [SMILES: CC1(C)C[C@@H]1C(=O)N1CC2(CN(C(=O)c3cncs3)CC2c2nnc(Cc3ccc(Cl)c(Cl)c3)o2)C1] |
| I-36 | 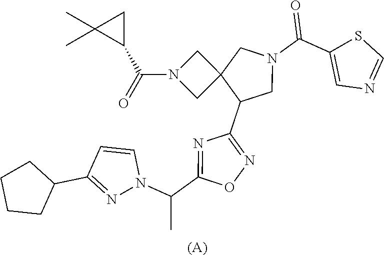 |
| | 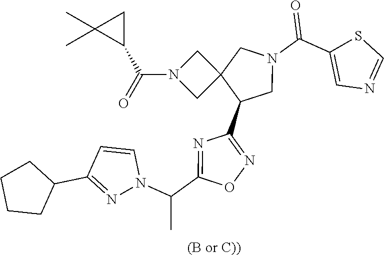 |
| | 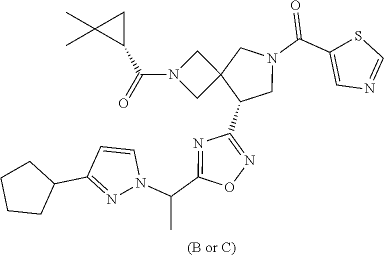 |
| | 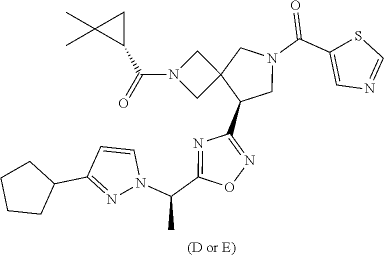 |
| | 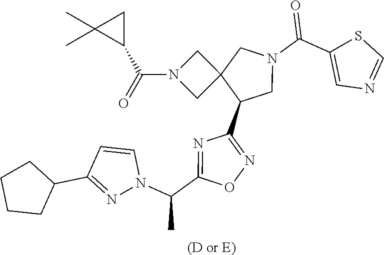 |
| I-37 | 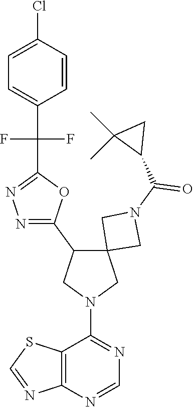 [SMILES: CC1(C)C[C@@H]1C(=O)N1CC2(C1)CN(c1ncnc3ncsc13)CC2c1nnc(C(F)(F)c2ccc(Cl)cc2)o1] |
| I-38 | 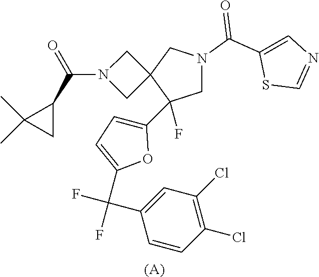 |
| | 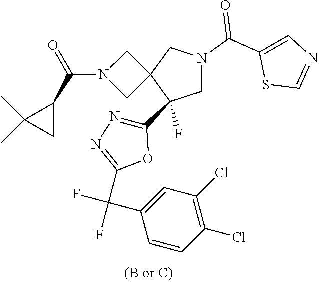 [SMILES: CC1(C)C[C@@H]1C(=O)N1CC2(CN(C(=O)c3cncs3)C[C@@]2(F)c2nnc(C(F)(F)c3ccc(Cl)c(Cl)c3)o2)C1] |
| | 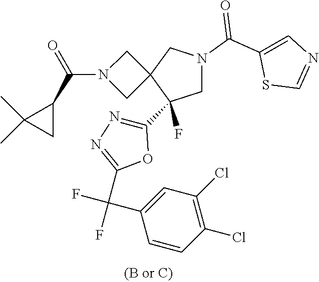 [SMILES: CC1(C)C[C@@H]1C(=O)N1CC2(CN(C(=O)c3cncs3)C[C@]2(F)c2nnc(C(F)(F)c3ccc(Cl)c(Cl)c3)o2)C1] |
| I-39 | 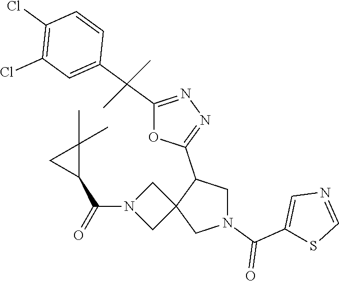 [SMILES: CC(C)(c1ccc(Cl)c(Cl)c1)c1nnc(C2CN(C(=O)c3cncs3)CC23CN(C(=O)[C@H]2CC2(C)C)C3)o1] |
| I-40 | 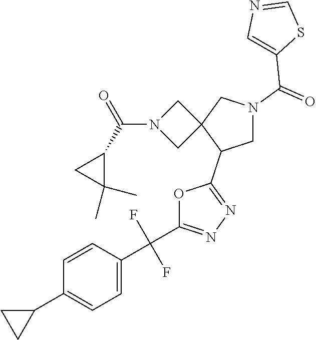 [SMILES: CC1(C)C[C@@H]1C(=O)N1CC2(CN(C(=O)c3cncs3)CC2c2nnc(C(F)(F)c3ccc(C4CC4)cc3)o2)C1] |
| I-41 | 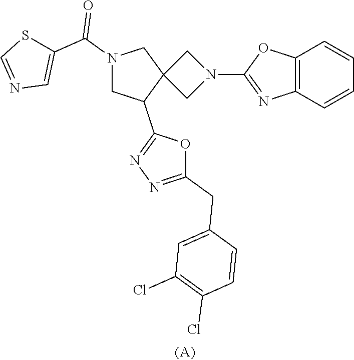 |
| | 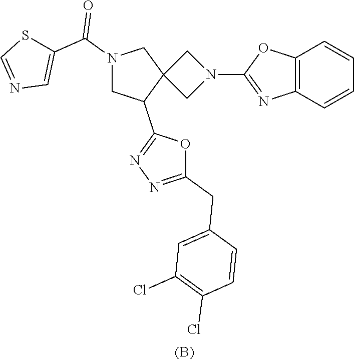 |
| I-42 | 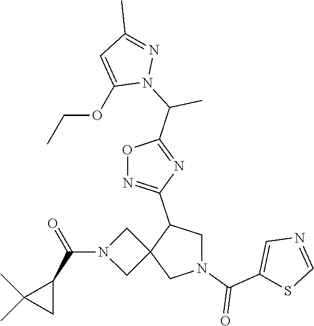 [SMILES: CCOc1cc(C)nn1C(C)c1nc(C2CN(C(=O)c3cncs3)CC23CN(C(=O)[C@H]2CC2(C)C)C3)no1] |
| I-43 | 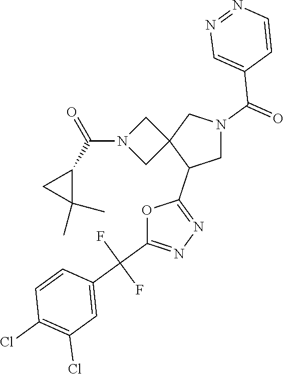 [SMILES: CC1(C)C[C@@H]1C(=O)N1CC2(CN(C(=O)c3ccnnc3)CC2c2nnc(C(F)(F)c3ccc(Cl)c(Cl)c3)o2)C1] |
| I-44 | 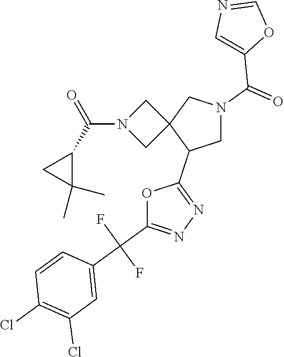 [SMILES: CC1(C)C[C@@H]1C(=O)N1CC2(CN(C(=O)c3cnco3)CC2c2nnc(C(F)(F)c3ccc(Cl)c(Cl)c3)o2)C1] |
| I-45 | 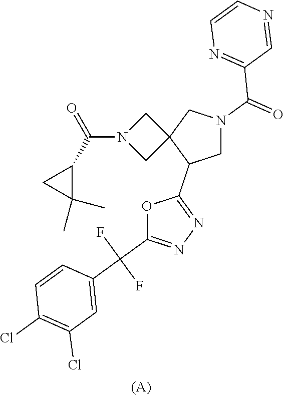 [SMILES: CC1(C)C[C@@H]1C(=O)N1CC2(CN(C(=O)c3cnccn3)CC2c2nnc(C(F)(F)c3ccc(Cl)c(Cl)c3)o2)C1 ; "(A)"] |
| | 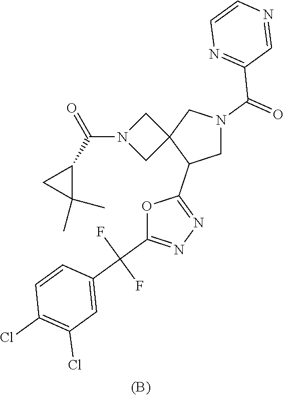 [SMILES: CC1(C)C[C@@H]1C(=O)N1CC2(CN(C(=O)c3cnccn3)CC2c2nnc(C(F)(F)c3ccc(Cl)c(Cl)c3)o2)C1 ; "(B)"] |
| I-46 | 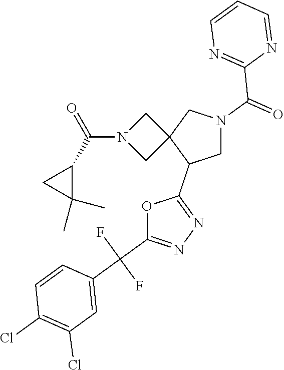 [SMILES: CC1(C)C[C@@H]1C(=O)N1CC2(CN(C(=O)c3ncccn3)CC2c2nnc(C(F)(F)c3ccc(Cl)c(Cl)c3)o2)C1] |
| I-47 | 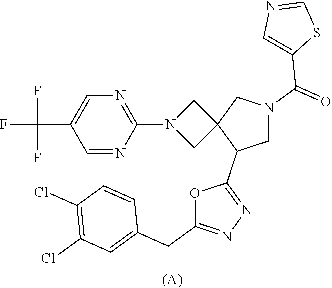 |
| | 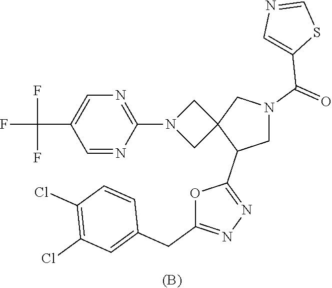 |
| I-48 | 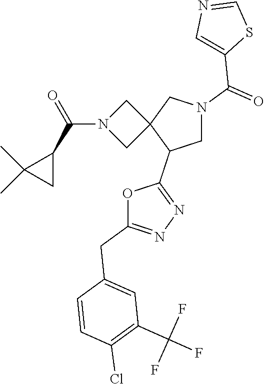 [SMILES: CC1(C)C[C@@H]1C(=O)N1CC2(CN(C(=O)c3cncs3)CC2c2nnc(Cc3ccc(Cl)c(C(F)(F)F)c3)o2)C1] |
| I-49 | 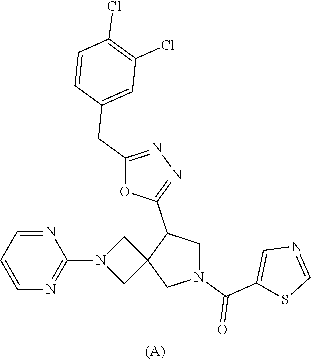 [SMILES: O=C(c1cncs1)N1CC(c2nnc(Cc3ccc(Cl)c(Cl)c3)o2)C2(C1)CN(c1ncccn1)C2 ; "(A)"] |
| | 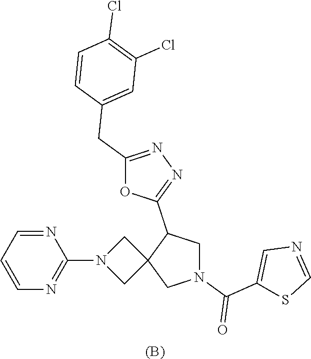 [SMILES: O=C(c1cncs1)N1CC(c2nnc(Cc3ccc(Cl)c(Cl)c3)o2)C2(C1)CN(c1ncccn1)C2 ; "(B)"] |
| I-50 | 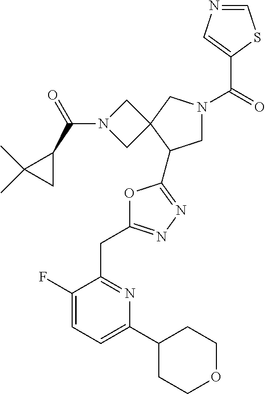 [SMILES: CC1(C)C[C@@H]1C(=O)N1CC2(CN(C(=O)c3cncs3)CC2c2nnc(Cc3nc(C4CCOCC4)ccc3F)o2)C1] |
| I-51 | 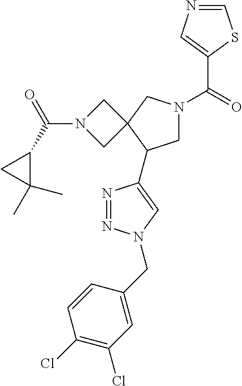 [SMILES: CC1(C)C[C@@H]1C(=O)N1CC2(CN(C(=O)c3cncs3)CC2c2cn(Cc3ccc(Cl)c(Cl)c3)nn2)C1] |
| I-52 | 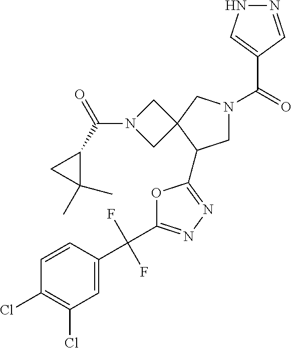 [SMILES: CC1(C)C[C@@H]1C(=O)N1CC2(CN(C(=O)c3cn[nH]c3)CC2c2nnc(C(F)(F)c3ccc(Cl)c(Cl)c3)o2)C1] |
| I-53 | 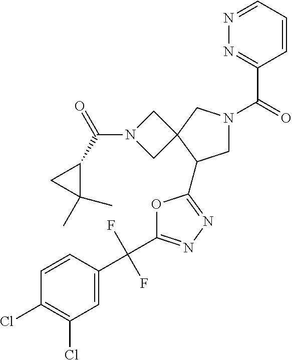 [SMILES: CC1(C)C[C@@H]1C(=O)N1CC2(CN(C(=O)c3cccnn3)CC2c2nnc(C(F)(F)c3ccc(Cl)c(Cl)c3)o2)C1] |
| I-54 | 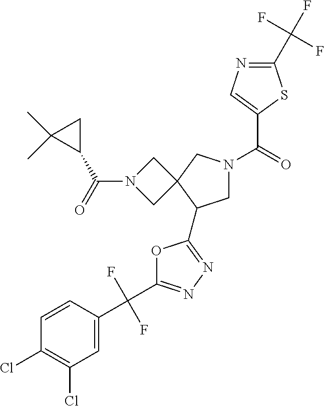 [SMILES: CC1(C)C[C@@H]1C(=O)N1CC2(CN(C(=O)c3cnc(C(F)(F)F)s3)CC2c2nnc(C(F)(F)c3ccc(Cl)c(Cl)c3)o2)C1] |
| I-55 | 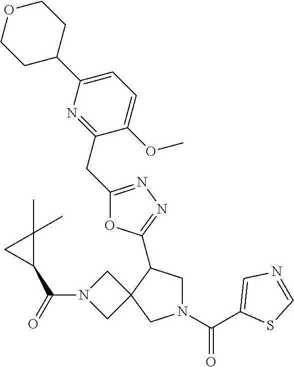 [SMILES: COc1ccc(C2CCOCC2)nc1Cc1nnc(C2CN(C(=O)c3cncs3)CC23CN(C(=O)[C@H]2CC2(C)C)C3)o1] |
| I-56 | 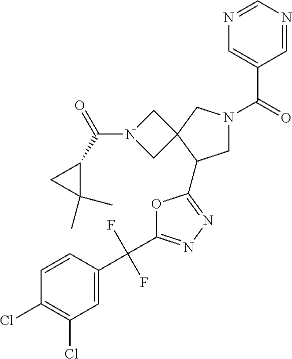 [SMILES: CC1(C)C[C@@H]1C(=O)N1CC2(CN(C(=O)c3cncnc3)CC2c2nnc(C(F)(F)c3ccc(Cl)c(Cl)c3)o2)C1] |
| I-57 | 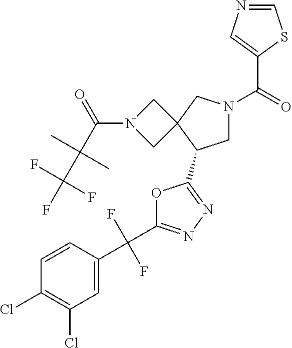 [SMILES: CC(C)(C(=O)N1CC2(CN(C(=O)c3cncs3)C[C@@H]2c2nnc(C(F)(F)c3ccc(Cl)c(Cl)c3)o2)C1)C(F)(F)F] |
| I-58 | 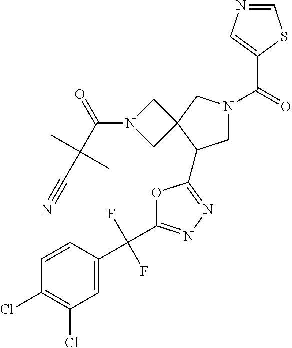 [SMILES: CC(C)(C#N)C(=O)N1CC2(CN(C(=O)c3cncs3)CC2c2nnc(C(F)(F)c3ccc(Cl)c(Cl)c3)o2)C1] |
| | (A) |
| | 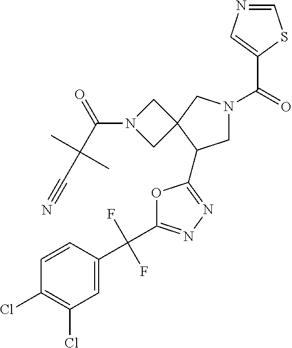 [SMILES: CC(C)(C#N)C(=O)N1CC2(CN(C(=O)c3cncs3)CC2c2nnc(C(F)(F)c3ccc(Cl)c(Cl)c3)o2)C1] |
| | (B) |
| I-59 | 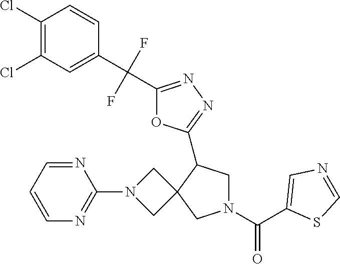 [SMILES: O=C(c1cncs1)N1CC(c2nnc(C(F)(F)c3ccc(Cl)c(Cl)c3)o2)C2(C1)CN(c1ncccn1)C2] |
| I-60 | 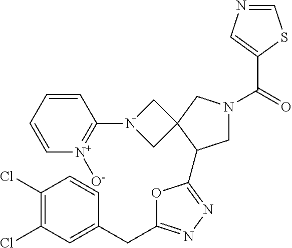 [SMILES: O=C(c1cncs1)N1CC(c2nnc(Cc3ccc(Cl)c(Cl)c3)o2)C2(C1)CN(c1cccc[n+]1[O-])C2] |
| I-61 | 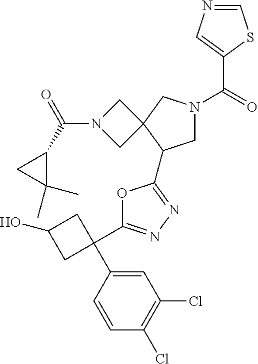 [SMILES: CC1(C)C[C@@H]1C(=O)N1CC2(CN(C(=O)c3cncs3)CC2c2nnc(C3(c4ccc(Cl)c(Cl)c4)CC(O)C3)o2)C1] |
| I-62 | 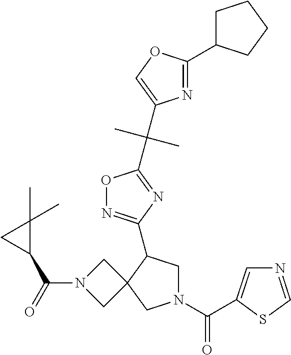 [SMILES: CC(C)(c1coc(C2CCCC2)n1)c1nc(C2CN(C(=O)c3cncs3)CC23CN(C(=O)[C@H]2CC2(C)C)C3)no1] |
| I-63 | 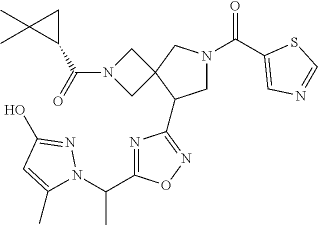 [SMILES: Cc1cc(O)nn1C(C)c1nc(C2CN(C(=O)c3cncs3)CC23CN(C(=O)[C@H]2CC2(C)C)C3)no1] |
| I-64 | 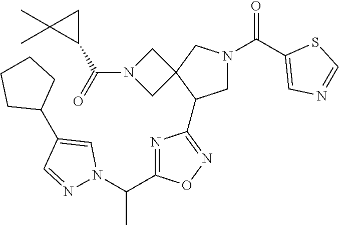 [SMILES: CC(c1nc(C2CN(C(=O)c3cncs3)CC23CN(C(=O)[C@H]2CC2(C)C)C3)no1)n1cc(C2CCCC2)cn1] |
| I-65 | 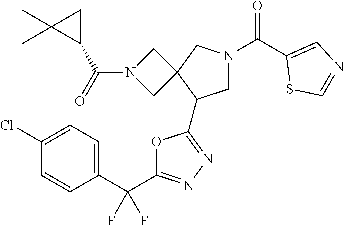 [SMILES: CC1(C)C[C@@H]1C(=O)N1CC2(CN(C(=O)c3cncs3)CC2c2nnc(C(F)(F)c3ccc(Cl)cc3)o2)C1] |
| I-66 | 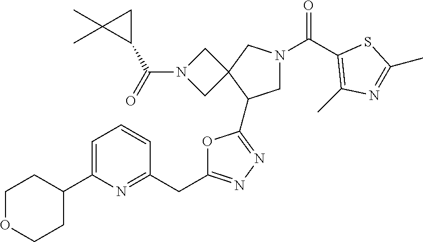 [SMILES: Cc1nc(C)c(C(=O)N2CC(c3nnc(Cc4cccc(C5CCOCC5)n4)o3)C3(C2)CN(C(=O)[C@H]2CC2(C)C)C3)s1] |
| I-67 | 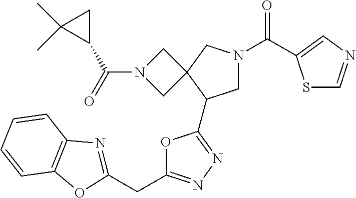 [SMILES: CC1(C)C[C@@H]1C(=O)N1CC2(CN(C(=O)c3cncs3)CC2c2nnc(Cc3nc4ccccc4o3)o2)C1] |
| I-68 | 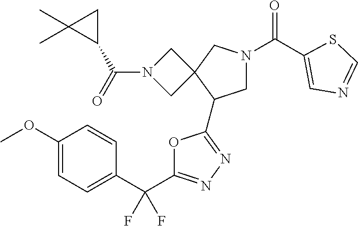 [SMILES: COc1ccc(C(F)(F)c2nnc(C3CN(C(=O)c4cncs4)CC34CN(C(=O)[C@H]3CC3(C)C)C4)o2)cc1] |
| I-69 | 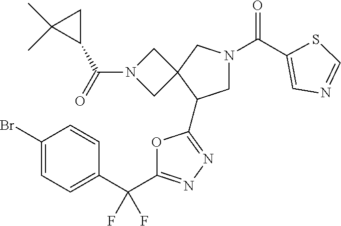 [SMILES: CC1(C)C[C@@H]1C(=O)N1CC2(CN(C(=O)c3cncs3)CC2c2nnc(C(F)(F)c3ccc(Br)cc3)o2)C1] |
| I-70 | 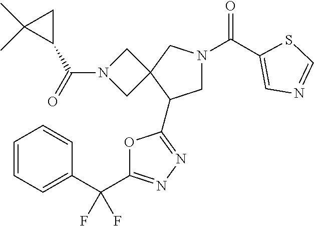 [SMILES: CC1(C)C[C@@H]1C(=O)N1CC2(CN(C(=O)c3cncs3)CC2c2nnc(C(F)(F)c3ccccc3)o2)C1] |
| I-71 | 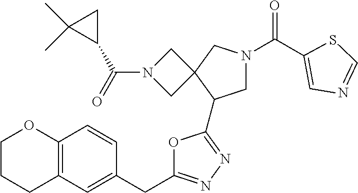 [SMILES: CC1(C)C[C@@H]1C(=O)N1CC2(CN(C(=O)c3cncs3)CC2c2nnc(Cc3ccc4c(c3)CCCO4)o2)C1] |
| I-72 | 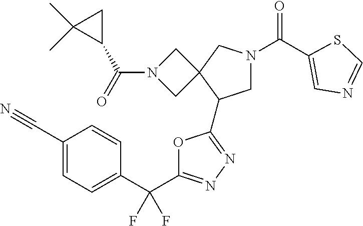 [SMILES: CC1(C)C[C@@H]1C(=O)N1CC2(CN(C(=O)c3cncs3)CC2c2nnc(C(F)(F)c3ccc(C#N)cc3)o2)C1] |
| I-73 | 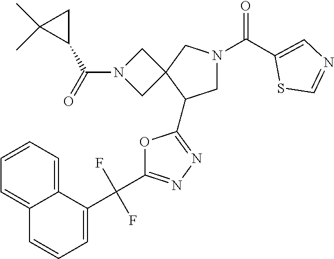 [SMILES: CC1(C)C[C@@H]1C(=O)N1CC2(CN(C(=O)c3cncs3)CC2c2nnc(C(F)(F)c3cccc4ccccc34)o2)C1] |
| I-74 | 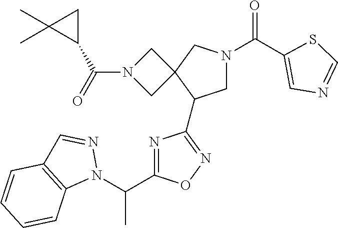 [SMILES: CC(c1nc(C2CN(C(=O)c3cncs3)CC23CN(C(=O)[C@H]2CC2(C)C)C3)no1)n1ncc2ccccc21] |
| I-75 | 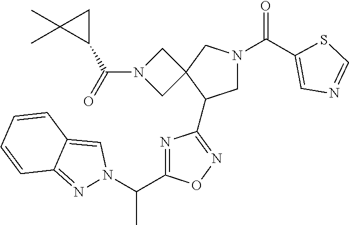 [SMILES: CC(c1nc(C2CN(C(=O)c3cncs3)CC23CN(C(=O)[C@H]2CC2(C)C)C3)no1)n1cc2ccccc2n1] |
| I-76 | 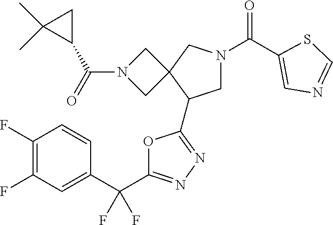 [SMILES: CC1(C)C[C@@H]1C(=O)N1CC2(CN(C(=O)c3cncs3)CC2c2nnc(C(F)(F)c3ccc(F)c(F)c3)o2)C1] |
| I-77 | 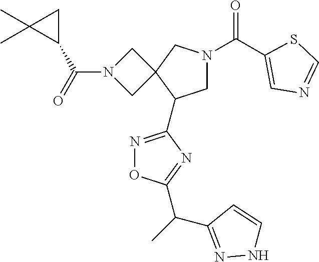 [SMILES: CC(c1cc[nH]n1)c1nc(C2CN(C(=O)c3cncs3)CC23CN(C(=O)[C@H]2CC2(C)C)C3)no1] |
| I-78 | 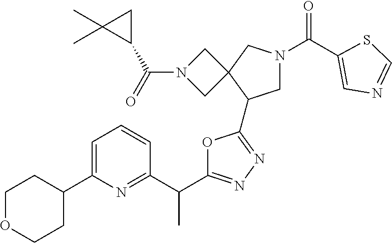 [SMILES: CC(c1cccc(C2CCOCC2)n1)c1nnc(C2CN(C(=O)c3cncs3)CC23CN(C(=O)[C@H]2CC2(C)C)C3)o1] |
| I-79 | 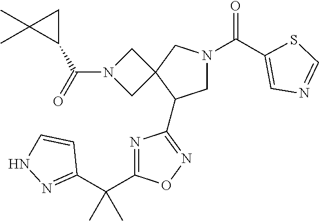 [SMILES: CC(C)(c1cc[nH]n1)c1nc(C2CN(C(=O)c3cncs3)CC23CN(C(=O)[C@H]2CC2(C)C)C3)no1] |
| I-80 | 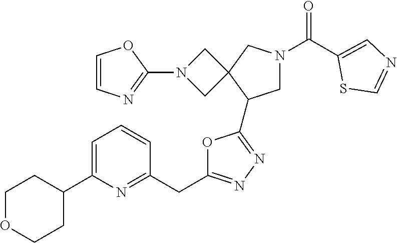 [SMILES: O=C(c1cncs1)N1CC(c2nnc(Cc3cccc(C4CCOCC4)n3)o2)C2(C1)CN(c1ncco1)C2] |
| | (A) |
| | 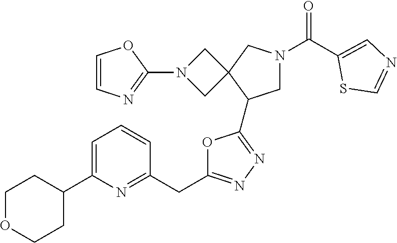 [SMILES: O=C(c1cncs1)N1CC(c2nnc(Cc3cccc(C4CCOCC4)n3)o2)C2(C1)CN(c1ncco1)C2] |
| | (B) |
| I-81 | 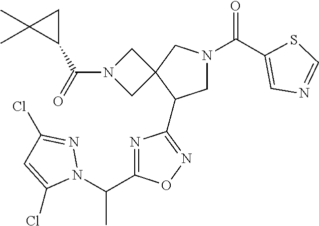 [SMILES: CC(c1nc(C2CN(C(=O)c3cncs3)CC23CN(C(=O)[C@H]2CC2(C)C)C3)no1)n1nc(Cl)cc1Cl] |
| I-82 | 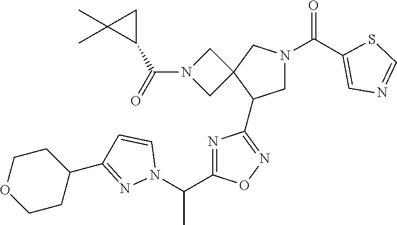 [SMILES: CC(c1nc(C2CN(C(=O)c3cncs3)CC23CN(C(=O)[C@H]2CC2(C)C)C3)no1)n1ccc(C2CCOCC2)n1] |
| I-83 | 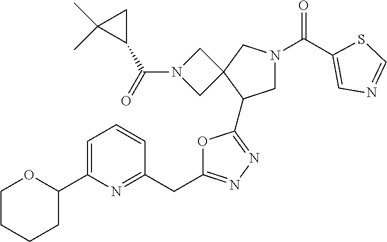 [SMILES: CC1(C)C[C@@H]1C(=O)N1CC2(CN(C(=O)c3cncs3)CC2c2nnc(Cc3cccc(C4CCCCO4)n3)o2)C1] |
| I-84 | 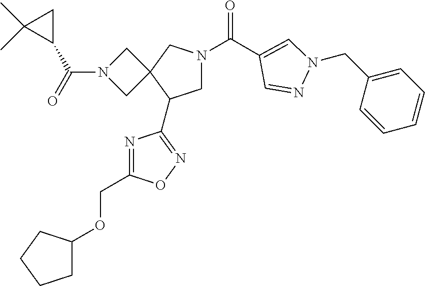 [SMILES: CC1(C)C[C@@H]1C(=O)N1CC2(CN(C(=O)c3cnn(Cc4ccccc4)c3)CC2c2noc(COC3CCCC3)n2)C1] |
| I-85 | 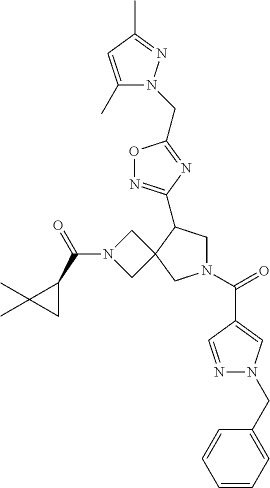 [SMILES: Cc1cc(C)n(Cc2nc(C3CN(C(=O)c4cnn(Cc5ccccc5)c4)CC34CN(C(=O)[C@H]3CC3(C)C)C4)no2)n1] |
| I-86 | 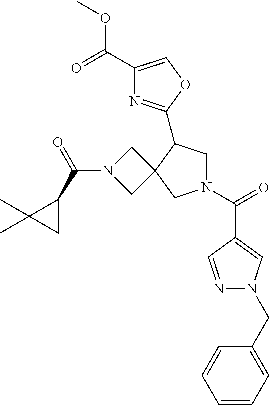 [SMILES: COC(=O)c1coc(C2CN(C(=O)c3cnn(Cc4ccccc4)c3)CC23CN(C(=O)[C@H]2CC2(C)C)C3)n1] |
| I-87 | 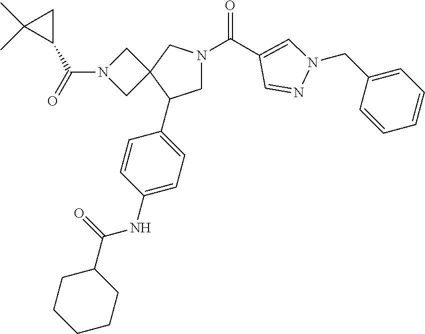 [SMILES: CC1(C)C[C@@H]1C(=O)N1CC2(CN(C(=O)c3cnn(Cc4ccccc4)c3)CC2c2ccc(NC(=O)C3CCCCC3)cc2)C1] |
| I-88 | 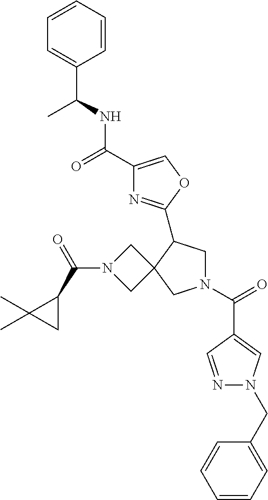 [SMILES: C[C@H](NC(=O)c1coc(C2CN(C(=O)c3cnn(Cc4ccccc4)c3)CC23CN(C(=O)[C@H]2CC2(C)C)C3)n1)c1ccccc1] |
| I-89 | 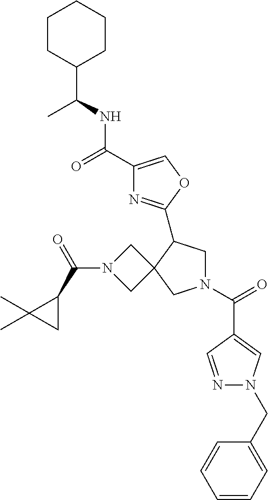 [SMILES: C[C@H](NC(=O)c1coc(C2CN(C(=O)c3cnn(Cc4ccccc4)c3)CC23CN(C(=O)[C@H]2CC2(C)C)C3)n1)C1CCCCC1] |
| I-90 | 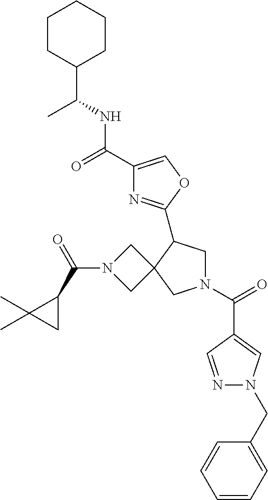 [SMILES: C[C@@H](NC(=O)c1coc(C2CN(C(=O)c3cnn(Cc4ccccc4)c3)CC23CN(C(=O)[C@H]2CC2(C)C)C3)n1)C1CCCCC1] |
| I-91 | 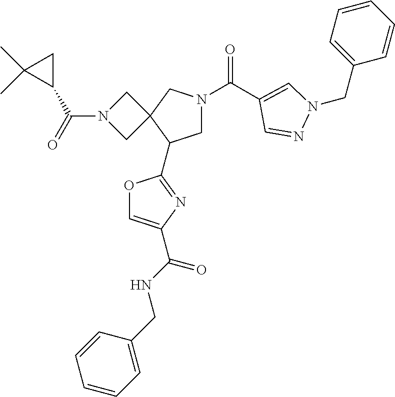 [SMILES: CC1(C)C[C@@H]1C(=O)N1CC2(CN(C(=O)c3cnn(Cc4ccccc4)c3)CC2c2nc(C(=O)NCc3ccccc3)co2)C1] |
| I-92 | 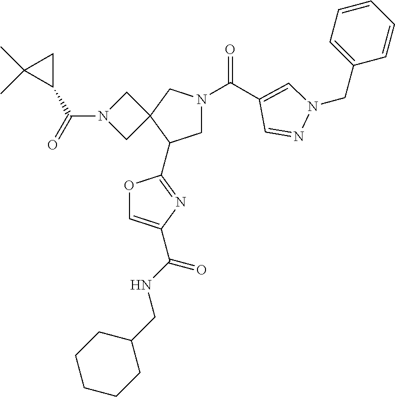 [SMILES: CC1(C)C[C@@H]1C(=O)N1CC2(CN(C(=O)c3cnn(Cc4ccccc4)c3)CC2c2nc(C(=O)NCC3CCCCC3)co2)C1] |
| I-93 | 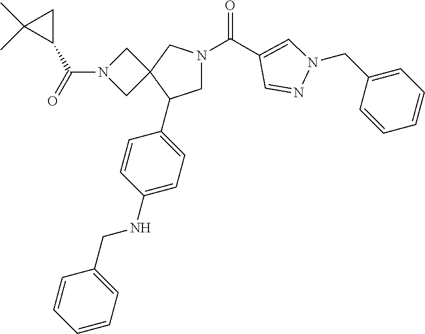 [SMILES: CC1(C)C[C@@H]1C(=O)N1CC2(CN(C(=O)c3cnn(Cc4ccccc4)c3)CC2c2ccc(NCc3ccccc3)cc2)C1] |
| I-94 | 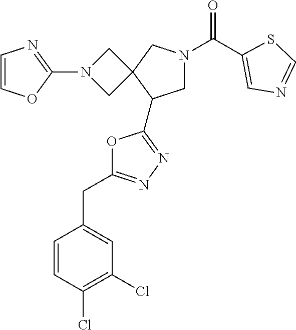 [SMILES: O=C(c1cncs1)N1CC(c2nnc(Cc3ccc(Cl)c(Cl)c3)o2)C2(C1)CN(c1ncco1)C2] |
| | (A) racemic |
| | 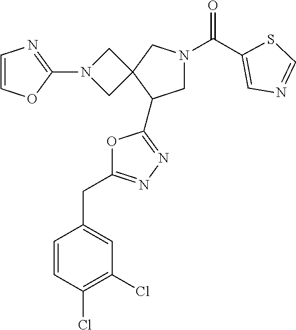 [SMILES: O=C(c1cncs1)N1CC(c2nnc(Cc3ccc(Cl)c(Cl)c3)o2)C2(C1)CN(c1ncco1)C2] |
| | (B) single enantiomer |
| | 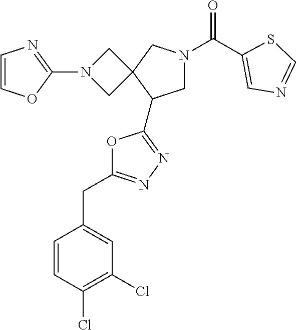 [SMILES: O=C(c1cncs1)N1CC(c2nnc(Cc3ccc(Cl)c(Cl)c3)o2)C2(C1)CN(c1ncco1)C2] |
| | (C) single enantiomer |
| I-95 | 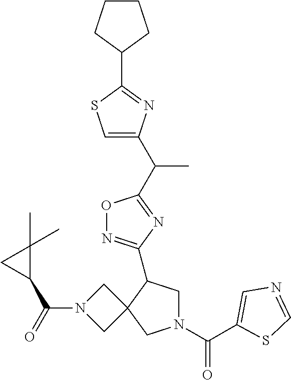 [SMILES: CC(c1csc(C2CCCC2)n1)c1nc(C2CN(C(=O)c3cncs3)CC23CN(C(=O)[C@H]2CC2(C)C)C3)no1] |
| I-96 | 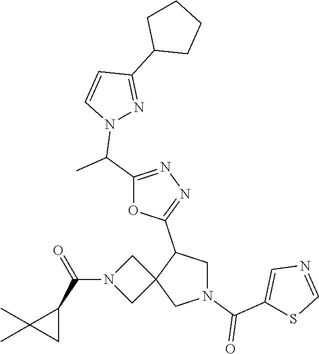 [SMILES: CC(c1nnc(C2CN(C(=O)c3cncs3)CC23CN(C(=O)[C@H]2CC2(C)C)C3)o1)n1ccc(C2CCCC2)n1] |
| I-97 | 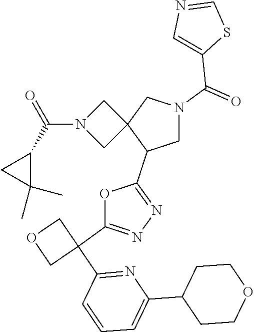 [SMILES: CC1(C)C[C@@H]1C(=O)N1CC2(CN(C(=O)c3cncs3)CC2c2nnc(C3(c4cccc(C5CCOCC5)n4)COC3)o2)C1] |
| I-98 | 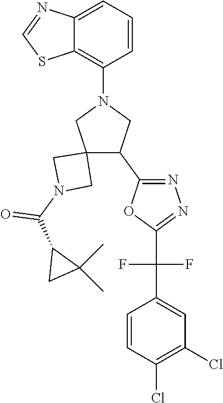 [SMILES: CC1(C)C[C@@H]1C(=O)N1CC2(C1)CN(c1cccc3ncsc13)CC2c1nnc(C(F)(F)c2ccc(Cl)c(Cl)c2)o1] |
| I-99 | 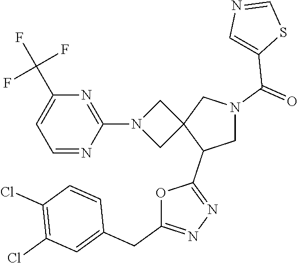 [SMILES: O=C(c1cncs1)N1CC(c2nnc(Cc3ccc(Cl)c(Cl)c3)o2)C2(C1)CN(c1nccc(C(F)(F)F)n1)C2] |
| | (A) |
| | 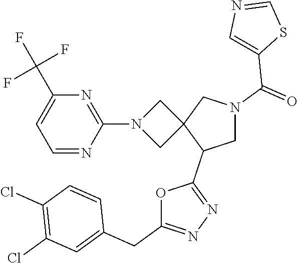 [SMILES: O=C(c1cncs1)N1CC(c2nnc(Cc3ccc(Cl)c(Cl)c3)o2)C2(C1)CN(c1nccc(C(F)(F)F)n1)C2] |
| | (B) |
| I-100 | 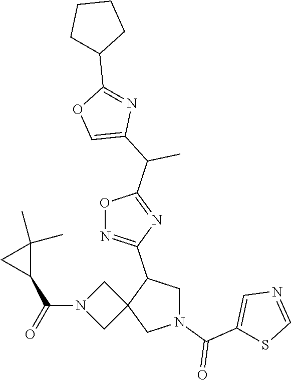 [SMILES: CC(c1coc(C2CCCC2)n1)c1nc(C2CN(C(=O)c3cncs3)CC23CN(C(=O)[C@H]2CC2(C)C)C3)no1] |
| I-101 | 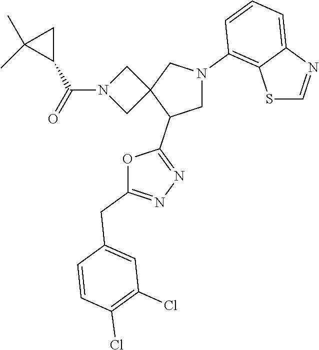 [SMILES: CC1(C)C[C@@H]1C(=O)N1CC2(C1)CN(c1cccc3ncsc13)CC2c1nnc(Cc2ccc(Cl)c(Cl)c2)o1] |
| I-102 | 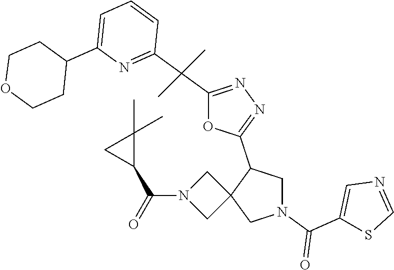 [SMILES: CC(C)(c1cccc(C2CCOCC2)n1)c1nnc(C2CN(C(=O)c3cncs3)CC23CN(C(=O)[C@H]2CC2(C)C)C3)o1] |
| I-103 | 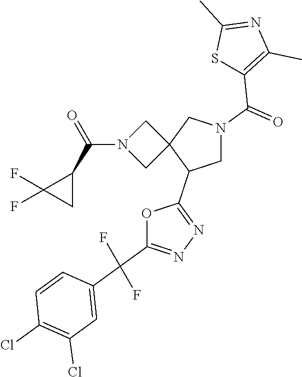 [SMILES: Cc1nc(C)c(C(=O)N2CC(c3nnc(C(F)(F)c4ccc(Cl)c(Cl)c4)o3)C3(C2)CN(C(=O)[C@H]2CC2(F)F)C3)s1] |
| I-104 | 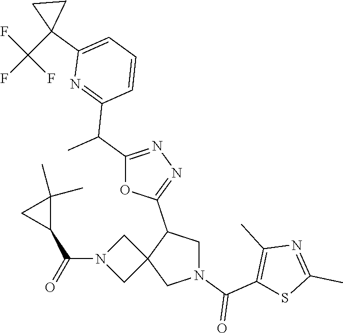 [SMILES: Cc1nc(C)c(C(=O)N2CC(c3nnc(C(C)c4cccc(C5(C(F)(F)F)CC5)n4)o3)C3(C2)CN(C(=O)[C@H]2CC2(C)C)C3)s1] |
| I-105 | 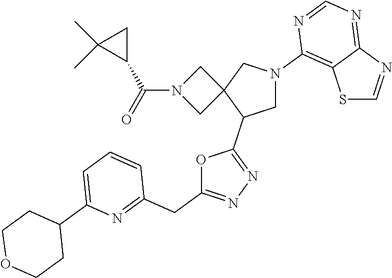 [SMILES: CC1(C)C[C@@H]1C(=O)N1CC2(C1)CN(c1ncnc3ncsc13)CC2c1nnc(Cc2cccc(C3CCOCC3)n2)o1] |
| I-106 | 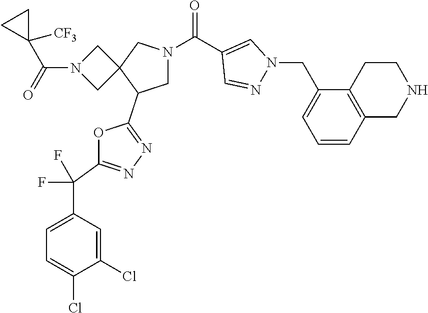 [SMILES: O=C(c1cnn(Cc2cccc3c2CCNC3)c1)N1CC(c2nnc(C(F)(F)c3ccc(Cl)c(Cl)c3)o2)C2(C1)CN(C(=O)C1(C(F)(F)F)CC1)C2] |
| I-107 | 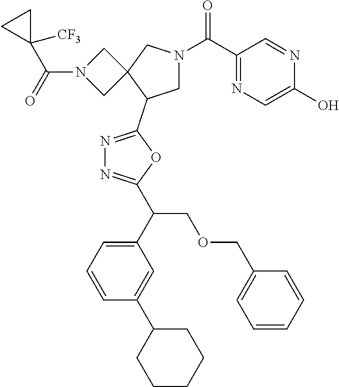 [SMILES: O=C(c1cnc(O)cn1)N1CC(c2nnc(C(COCc3ccccc3)c3cccc(C4CCCCC4)c3)o2)C2(C1)CN(C(=O)C1(C(F)(F)F)CC1)C2] |
| I-108 | 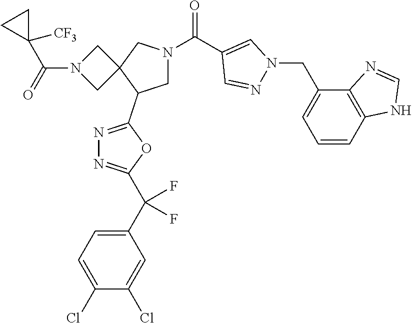 [SMILES: O=C(c1cnn(Cc2cccc3[nH]cnc23)c1)N1CC(c2nnc(C(F)(F)c3ccc(Cl)c(Cl)c3)o2)C2(C1)CN(C(=O)C1(C(F)(F)F)CC1)C2] |
| I-109 | 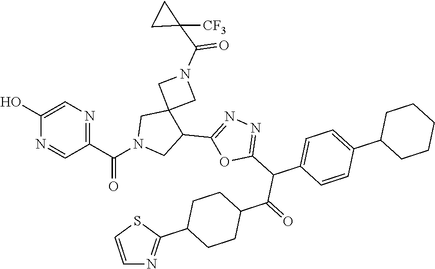 [SMILES: O=C(C1CCC(c2nccs2)CC1)C(c1ccc(C2CCCCC2)cc1)c1nnc(C2CN(C(=O)c3cnc(O)cn3)CC23CN(C(=O)C2(C(F)(F)F)CC2)C3)o1] |
| I-110 | 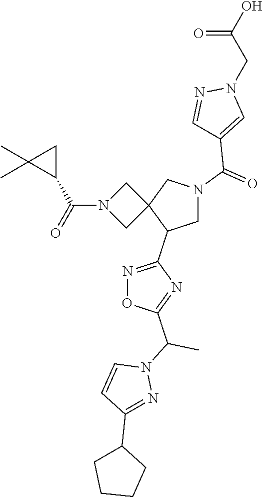 [SMILES: CC(c1nc(C2CN(C(=O)c3cnn(CC(=O)O)c3)CC23CN(C(=O)[C@H]2CC2(C)C)C3)no1)n1ccc(C2CCCC2)n1] |
| I-111 | 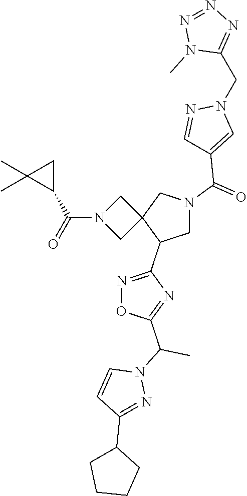 [SMILES: CC(c1nc(C2CN(C(=O)c3cnn(Cc4nnnn4C)c3)CC23CN(C(=O)[C@H]2CC2(C)C)C3)no1)n1ccc(C2CCCC2)n1] |
| I-112 | 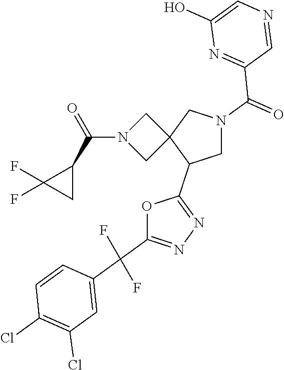 [SMILES: O=C(c1cncc(O)n1)N1CC(c2nnc(C(F)(F)c3ccc(Cl)c(Cl)c3)o2)C2(C1)CN(C(=O)[C@H]1CC1(F)F)C2] |
| I-113 | 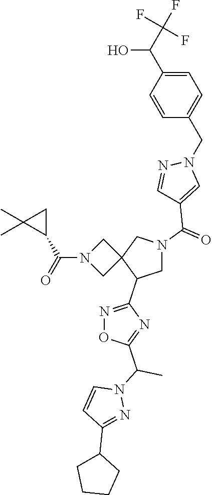 [SMILES: CC(c1nc(C2CN(C(=O)c3cnn(Cc4ccc(C(O)C(F)(F)F)cc4)c3)CC23CN(C(=O)[C@H]2CC2(C)C)C3)no1)n1ccc(C2CCCC2)n1] |
| | (A) |
| | 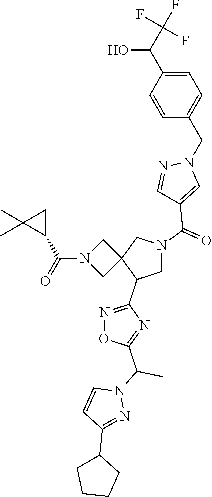 [SMILES: CC(c1nc(C2CN(C(=O)c3cnn(Cc4ccc(C(O)C(F)(F)F)cc4)c3)CC23CN(C(=O)[C@H]2CC2(C)C)C3)no1)n1ccc(C2CCCC2)n1] |
| | (B) |
| I-114 | 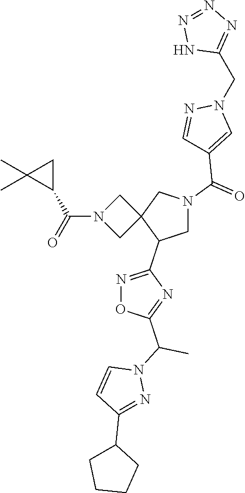 [SMILES: CC(c1nc(C2CN(C(=O)c3cnn(Cc4nnn[nH]4)c3)CC23CN(C(=O)[C@H]2CC2(C)C)C3)no1)n1ccc(C2CCCC2)n1] |
| I-115 | 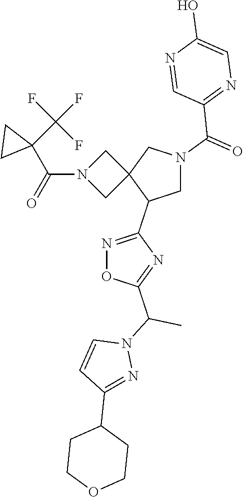 [SMILES: CC(c1nc(C2CN(C(=O)c3cnc(O)cn3)CC23CN(C(=O)C2(C(F)(F)F)CC2)C3)no1)n1ccc(C2CCOCC2)n1] |
| I-116 | 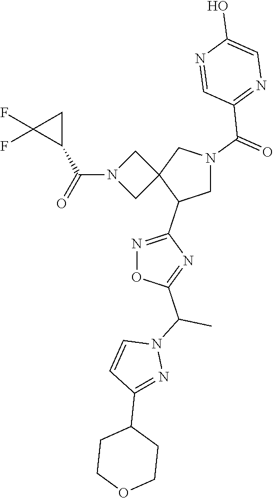 [SMILES: CC(c1nc(C2CN(C(=O)c3cnc(O)cn3)CC23CN(C(=O)[C@H]2CC2(F)F)C3)no1)n1ccc(C2CCOCC2)n1] |
| I-117 | 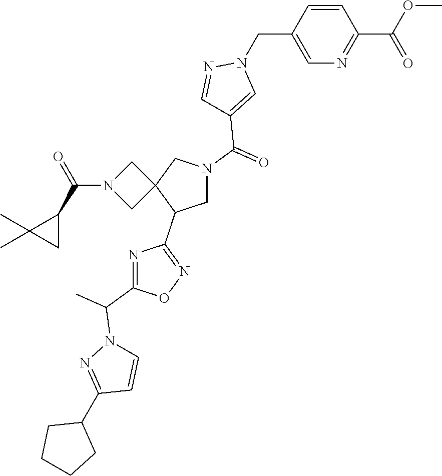 |
| I-118 | 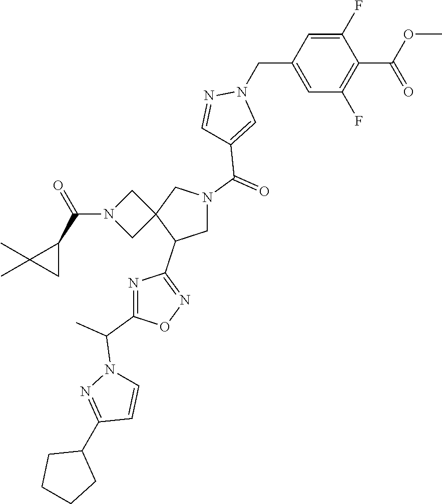 |
| I-119 | 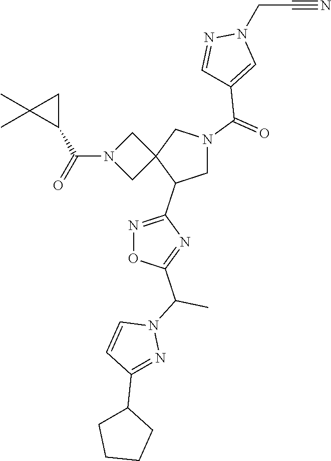 [SMILES: CC(c1nc(C2CN(C(=O)c3cnn(CC#N)c3)CC23CN(C(=O)[C@H]2CC2(C)C)C3)no1)n1ccc(C2CCCC2)n1] |
| I-120 | 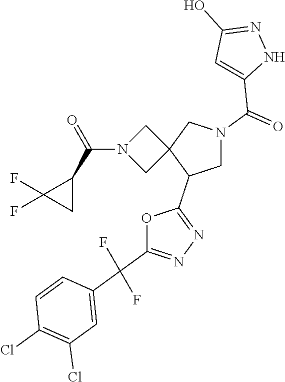 [SMILES: O=C(c1cc(O)n[nH]1)N1CC(c2nnc(C(F)(F)c3ccc(Cl)c(Cl)c3)o2)C2(C1)CN(C(=O)[C@H]1CC1(F)F)C2] |
| I-121 | 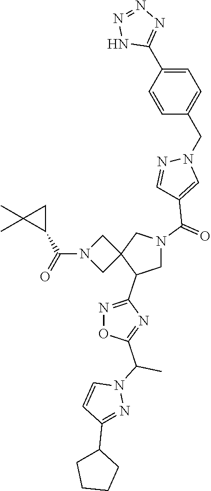 [SMILES: CC(c1nc(C2CN(C(=O)c3cnn(Cc4ccc(-c5nnn[nH]5)cc4)c3)CC23CN(C(=O)[C@H]2CC2(C)C)C3)no1)n1ccc(C2CCCC2)n1] |
| I-122 | 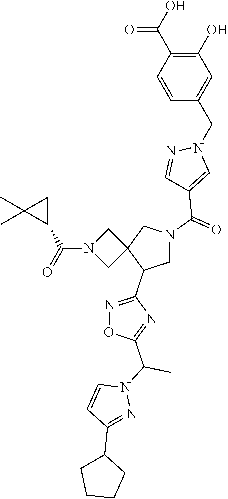 [SMILES: CC(c1nc(C2CN(C(=O)c3cnn(Cc4ccc(C(=O)O)c(O)c4)c3)CC23CN(C(=O)[C@H]2CC2(C)C)C3)no1)n1ccc(C2CCCC2)n1] |
| I-123 | 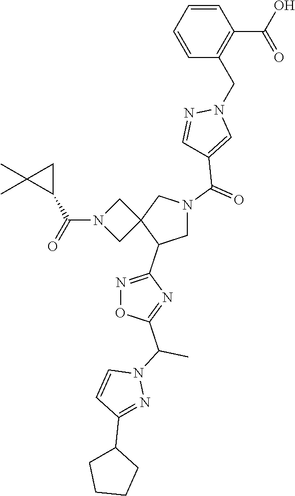 [SMILES: CC(c1nc(C2CN(C(=O)c3cnn(Cc4ccccc4C(=O)O)c3)CC23CN(C(=O)[C@H]2CC2(C)C)C3)no1)n1ccc(C2CCCC2)n1] |
| I-124 | 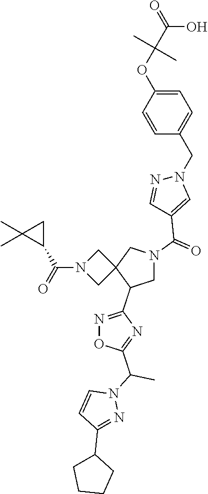 [SMILES: CC(c1nc(C2CN(C(=O)c3cnn(Cc4ccc(OC(C)(C)C(=O)O)cc4)c3)CC23CN(C(=O)[C@H]2CC2(C)C)C3)no1)n1ccc(C2CCCC2)n1] |
| I-125 | 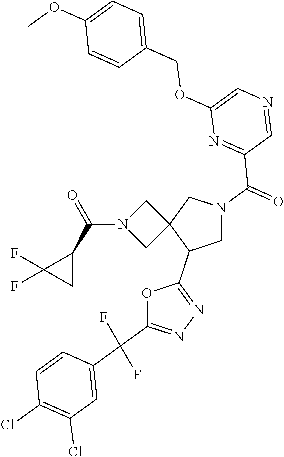 [SMILES: COc1ccc(COc2cncc(C(=O)N3CC(c4nnc(C(F)(F)c5ccc(Cl)c(Cl)c5)o4)C4(C3)CN(C(=O)[C@H]3CC3(F)F)C4)n2)cc1] |
| I-126 | 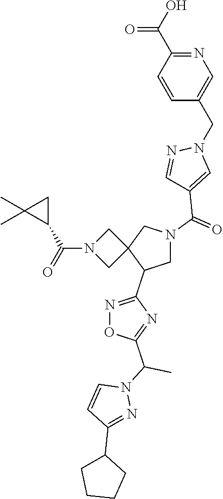 [SMILES: CC(c1nc(C2CN(C(=O)c3cnn(Cc4ccc(C(=O)O)nc4)c3)CC23CN(C(=O)[C@H]2CC2(C)C)C3)no1)n1ccc(C2CCCC2)n1] |
| I-127 | 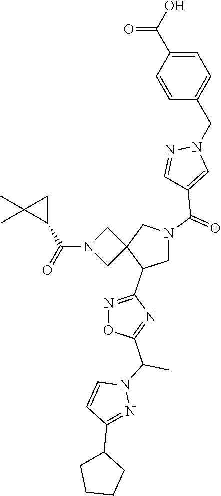 [SMILES: CC(c1nc(C2CN(C(=O)c3cnn(Cc4ccc(C(=O)O)cc4)c3)CC23CN(C(=O)[C@H]2CC2(C)C)C3)no1)n1ccc(C2CCCC2)n1] |
| I-128 | 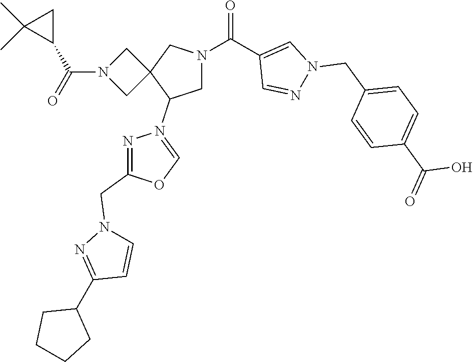 |
| I-129 | 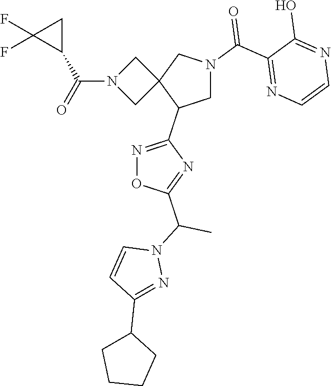 [SMILES: CC(c1nc(C2CN(C(=O)c3nccnc3O)CC23CN(C(=O)[C@H]2CC2(F)F)C3)no1)n1ccc(C2CCCC2)n1] |
| I-130 | 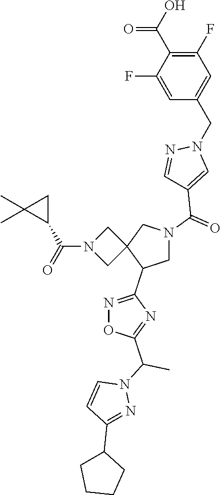 [SMILES: CC(c1nc(C2CN(C(=O)c3cnn(Cc4cc(F)c(C(=O)O)c(F)c4)c3)CC23CN(C(=O)[C@H]2CC2(C)C)C3)no1)n1ccc(C2CCCC2)n1] |
| I-131 | 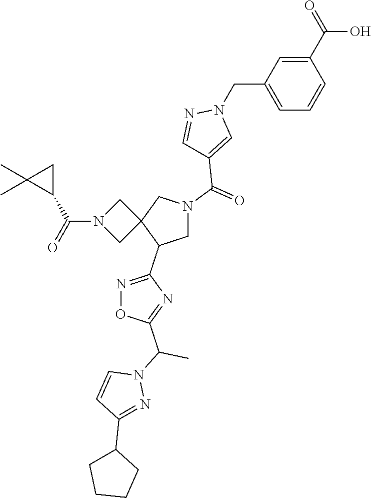 [SMILES: CC(c1nc(C2CN(C(=O)c3cnn(Cc4cccc(C(=O)O)c4)c3)CC23CN(C(=O)[C@H]2CC2(C)C)C3)no1)n1ccc(C2CCCC2)n1] |
| I-132 | 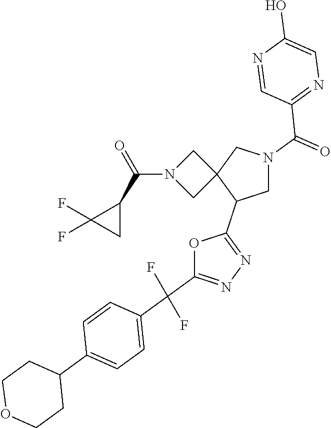 [SMILES: O=C(c1cnc(O)cn1)N1CC(c2nnc(C(F)(F)c3ccc(C4CCOCC4)cc3)o2)C2(C1)CN(C(=O)[C@H]1CC1(F)F)C2] |
| I-133 | 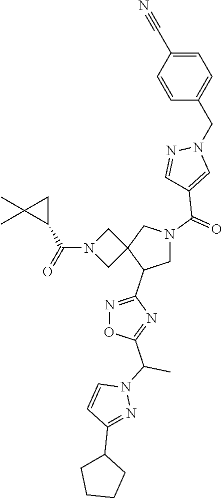 [SMILES: CC(c1nc(C2CN(C(=O)c3cnn(Cc4ccc(C#N)cc4)c3)CC23CN(C(=O)[C@H]2CC2(C)C)C3)no1)n1ccc(C2CCCC2)n1] |
| I-134 | 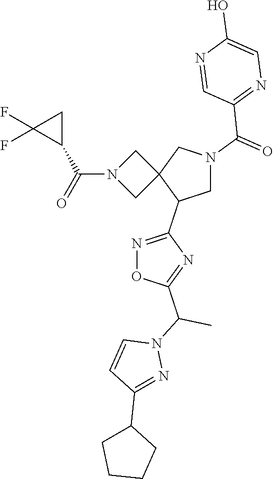 [SMILES: CC(c1nc(C2CN(C(=O)c3cnc(O)cn3)CC23CN(C(=O)[C@H]2CC2(F)F)C3)no1)n1ccc(C2CCCC2)n1] |
| I-135 | 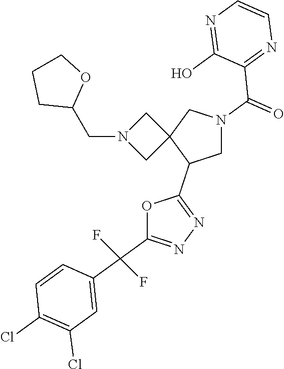 [SMILES: O=C(c1nccnc1O)N1CC(c2nnc(C(F)(F)c3ccc(Cl)c(Cl)c3)o2)C2(CN(CC3CCCO3)C2)C1] |
| I-136 | 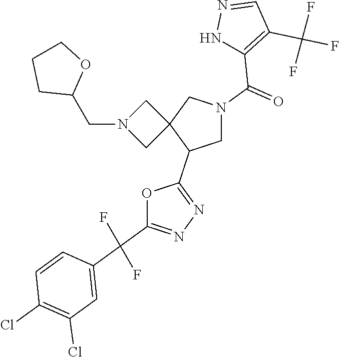 [SMILES: O=C(c1[nH]ncc1C(F)(F)F)N1CC(c2nnc(C(F)(F)c3ccc(Cl)c(Cl)c3)o2)C2(CN(CC3CCCO3)C2)C1] |
| I-137 | 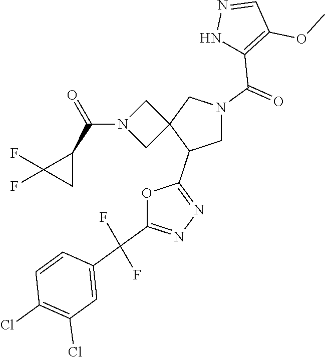 [SMILES: COc1cn[nH]c1C(=O)N1CC(c2nnc(C(F)(F)c3ccc(Cl)c(Cl)c3)o2)C2(C1)CN(C(=O)[C@H]1CC1(F)F)C2] |
| I-138 | 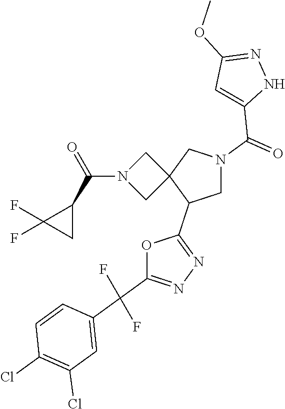 [SMILES: COc1cc(C(=O)N2CC(c3nnc(C(F)(F)c4ccc(Cl)c(Cl)c4)o3)C3(C2)CN(C(=O)[C@H]2CC2(F)F)C3)[nH]n1] |
| I-139 | 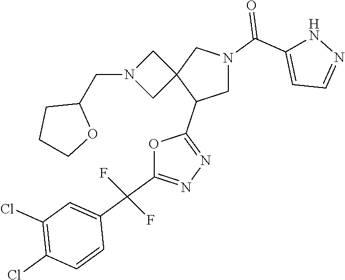 [SMILES: O=C(c1ccn[nH]1)N1CC(c2nnc(C(F)(F)c3ccc(Cl)c(Cl)c3)o2)C2(CN(CC3CCCO3)C2)C1] |
| I-140 | 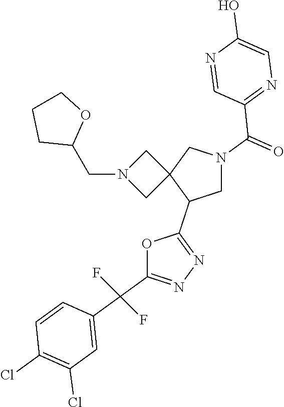 [SMILES: O=C(c1cnc(O)cn1)N1CC(c2nnc(C(F)(F)c3ccc(Cl)c(Cl)c3)o2)C2(CN(CC3CCCO3)C2)C1] |
| I-141 | 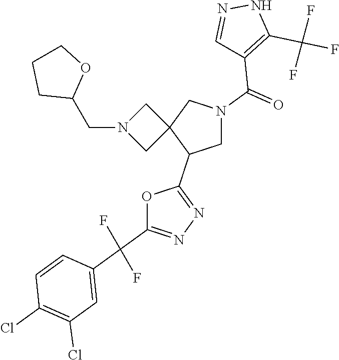 [SMILES: O=C(c1cn[nH]c1C(F)(F)F)N1CC(c2nnc(C(F)(F)c3ccc(Cl)c(Cl)c3)o2)C2(CN(CC3CCCO3)C2)C1] |
| I-142 | 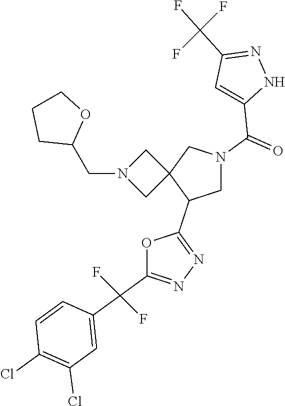 [SMILES: O=C(c1cc(C(F)(F)F)n[nH]1)N1CC(c2nnc(C(F)(F)c3ccc(Cl)c(Cl)c3)o2)C2(CN(CC3CCCO3)C2)C1] |
| I-143 | 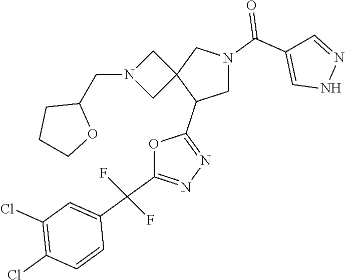 [SMILES: O=C(c1cn[nH]c1)N1CC(c2nnc(C(F)(F)c3ccc(Cl)c(Cl)c3)o2)C2(CN(CC3CCCO3)C2)C1] |
| I-144 | 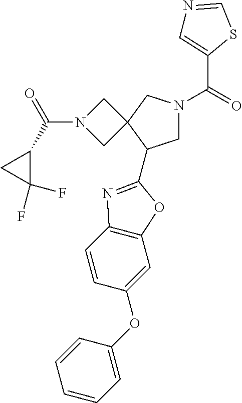 [SMILES: O=C(c1cncs1)N1CC(c2nc3ccc(Oc4ccccc4)cc3o2)C2(C1)CN(C(=O)[C@H]1CC1(F)F)C2] |
| I-145 | 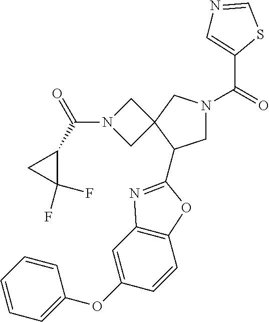 [SMILES: O=C(c1cncs1)N1CC(c2nc3cc(Oc4ccccc4)ccc3o2)C2(C1)CN(C(=O)[C@H]1CC1(F)F)C2] |
| I-146 | 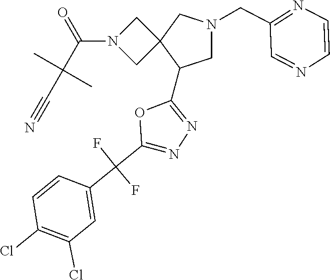 [SMILES: CC(C)(C#N)C(=O)N1CC2(CN(Cc3cnccn3)CC2c2nnc(C(F)(F)c3ccc(Cl)c(Cl)c3)o2)C1] |
| I-147 | 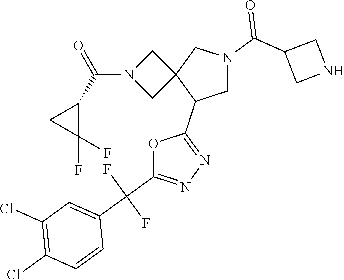 [SMILES: O=C(C1CNC1)N1CC(c2nnc(C(F)(F)c3ccc(Cl)c(Cl)c3)o2)C2(C1)CN(C(=O)[C@H]1CC1(F)F)C2] |
| I-148 | 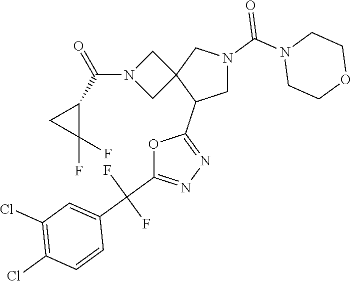 [SMILES: O=C([C@H]1CC1(F)F)N1CC2(C1)CN(C(=O)N1CCOCC1)CC2c1nnc(C(F)(F)c2ccc(Cl)c(Cl)c2)o1] |
| I-149 | 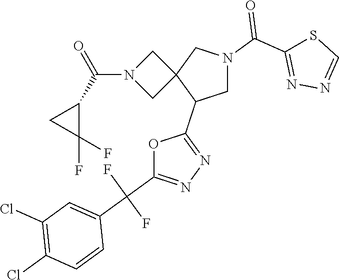 [SMILES: O=C(c1nncs1)N1CC(c2nnc(C(F)(F)c3ccc(Cl)c(Cl)c3)o2)C2(C1)CN(C(=O)[C@H]1CC1(F)F)C2] |
| I-150 | 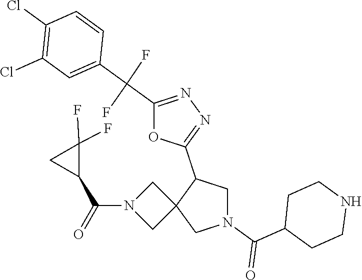 [SMILES: O=C(C1CCNCC1)N1CC(c2nnc(C(F)(F)c3ccc(Cl)c(Cl)c3)o2)C2(C1)CN(C(=O)[C@H]1CC1(F)F)C2] |
| I-151 | 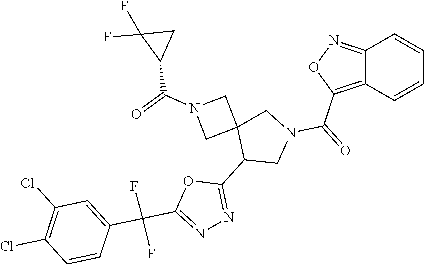 [SMILES: O=C(c1onc2ccccc12)N1CC(c2nnc(C(F)(F)c3ccc(Cl)c(Cl)c3)o2)C2(C1)CN(C(=O)[C@H]1CC1(F)F)C2] |
| | (A) |
| | 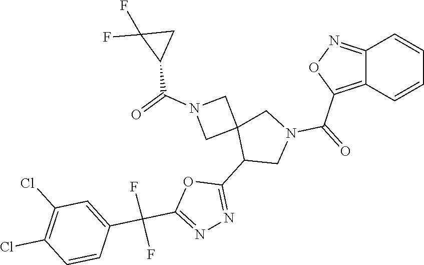 [SMILES: O=C(c1onc2ccccc12)N1CC(c2nnc(C(F)(F)c3ccc(Cl)c(Cl)c3)o2)C2(C1)CN(C(=O)[C@H]1CC1(F)F)C2] |
| | (B) |
| I-152 | 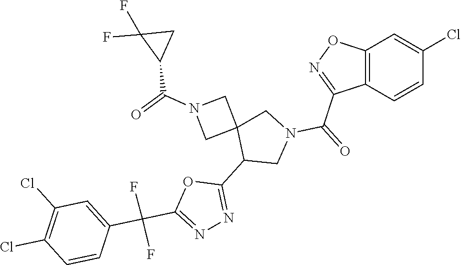 [SMILES: O=C(c1noc2cc(Cl)ccc12)N1CC(c2nnc(C(F)(F)c3ccc(Cl)c(Cl)c3)o2)C2(C1)CN(C(=O)[C@H]1CC1(F)F)C2] |
| I-153 | 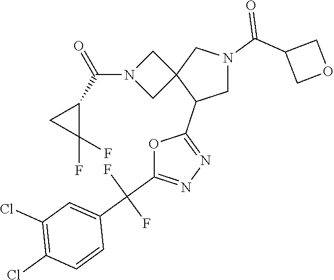 [SMILES: O=C(C1COC1)N1CC(c2nnc(C(F)(F)c3ccc(Cl)c(Cl)c3)o2)C2(C1)CN(C(=O)[C@H]1CC1(F)F)C2] |
| I-154 | 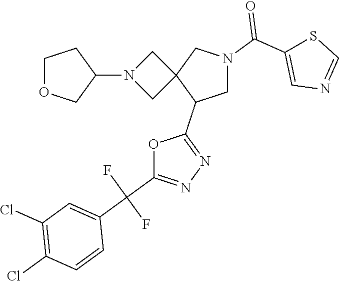 [SMILES: O=C(c1cncs1)N1CC(c2nnc(C(F)(F)c3ccc(Cl)c(Cl)c3)o2)C2(C1)CN(C1CCOC1)C2] |
| I-155 | 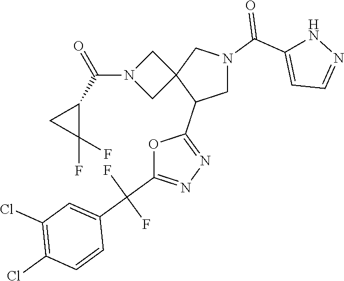 [SMILES: O=C(c1ccn[nH]1)N1CC(c2nnc(C(F)(F)c3ccc(Cl)c(Cl)c3)o2)C2(C1)CN(C(=O)[C@H]1CC1(F)F)C2] |
| I-156 | 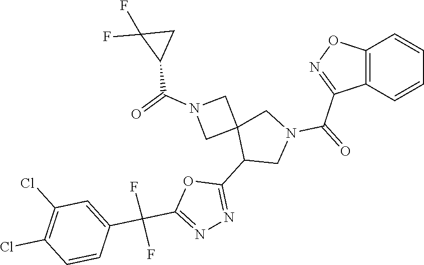 [SMILES: O=C(c1noc2ccccc12)N1CC(c2nnc(C(F)(F)c3ccc(Cl)c(Cl)c3)o2)C2(C1)CN(C(=O)[C@H]1CC1(F)F)C2] |
| I-157 | 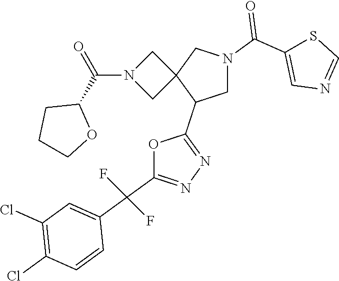 [SMILES: O=C(c1cncs1)N1CC(c2nnc(C(F)(F)c3ccc(Cl)c(Cl)c3)o2)C2(C1)CN(C(=O)[C@H]1CCCO1)C2] |
| I-158 | 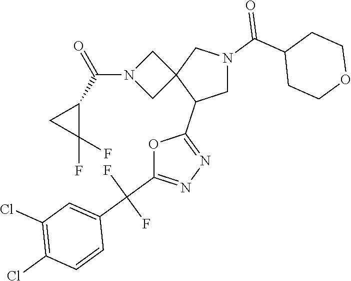 [SMILES: O=C(C1CCOCC1)N1CC(c2nnc(C(F)(F)c3ccc(Cl)c(Cl)c3)o2)C2(C1)CN(C(=O)[C@H]1CC1(F)F)C2] |
| I-159 | 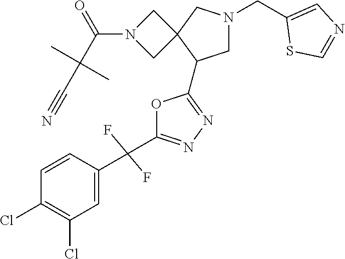 [SMILES: CC(C)(C#N)C(=O)N1CC2(CN(Cc3cncs3)CC2c2nnc(C(F)(F)c3ccc(Cl)c(Cl)c3)o2)C1] |
| I-160 | 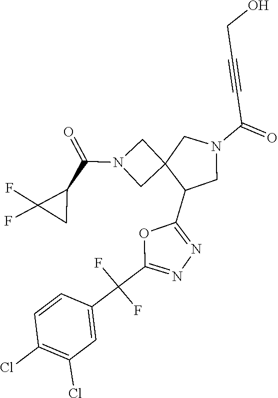 [SMILES: O=C(C#CCO)N1CC(c2nnc(C(F)(F)c3ccc(Cl)c(Cl)c3)o2)C2(C1)CN(C(=O)[C@H]1CC1(F)F)C2] |
| I-161 | 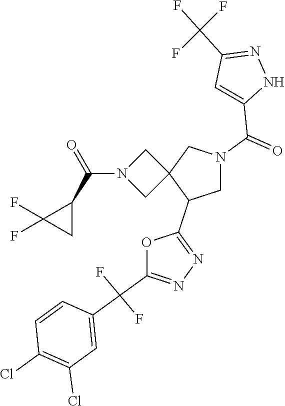 [SMILES: O=C(c1cc(C(F)(F)F)n[nH]1)N1CC(c2nnc(C(F)(F)c3ccc(Cl)c(Cl)c3)o2)C2(C1)CN(C(=O)[C@H]1CC1(F)F)C2] |
| I-162 | 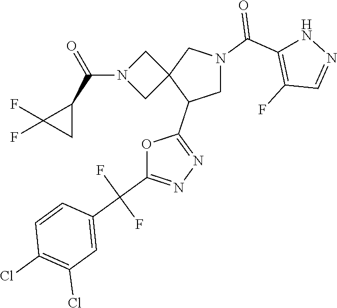 [SMILES: O=C(c1[nH]ncc1F)N1CC(c2nnc(C(F)(F)c3ccc(Cl)c(Cl)c3)o2)C2(C1)CN(C(=O)[C@H]1CC1(F)F)C2] |
| I-163 | 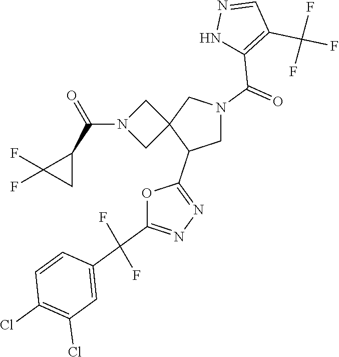 [SMILES: O=C(c1[nH]ncc1C(F)(F)F)N1CC(c2nnc(C(F)(F)c3ccc(Cl)c(Cl)c3)o2)C2(C1)CN(C(=O)[C@H]1CC1(F)F)C2] |
| I-164 | 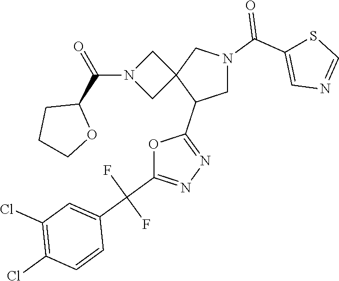 [SMILES: O=C(c1cncs1)N1CC(c2nnc(C(F)(F)c3ccc(Cl)c(Cl)c3)o2)C2(C1)CN(C(=O)[C@@H]1CCCO1)C2] |
| I-165 | 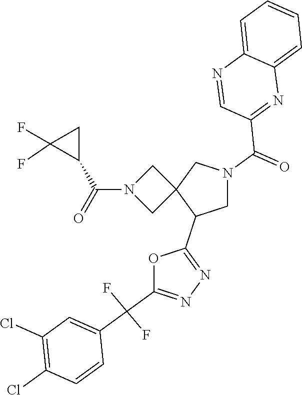 [SMILES: O=C(c1cnc2ccccc2n1)N1CC(c2nnc(C(F)(F)c3ccc(Cl)c(Cl)c3)o2)C2(C1)CN(C(=O)[C@H]1CC1(F)F)C2] |
| I-166 | 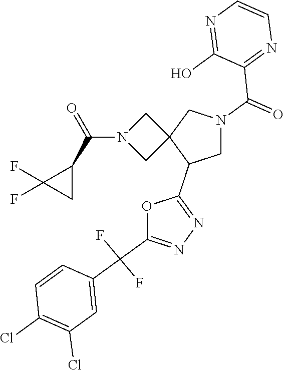 [SMILES: O=C(c1nccnc1O)N1CC(c2nnc(C(F)(F)c3ccc(Cl)c(Cl)c3)o2)C2(C1)CN(C(=O)[C@H]1CC1(F)F)C2] |
| I-167 | 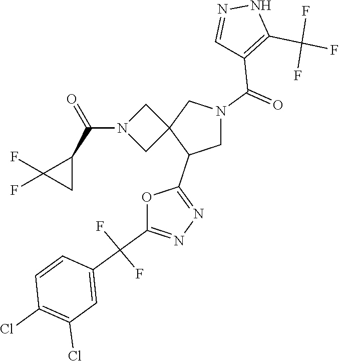 [SMILES: O=C(c1cn[nH]c1C(F)(F)F)N1CC(c2nnc(C(F)(F)c3ccc(Cl)c(Cl)c3)o2)C2(C1)CN(C(=O)[C@H]1CC1(F)F)C2] |
| I-168 | 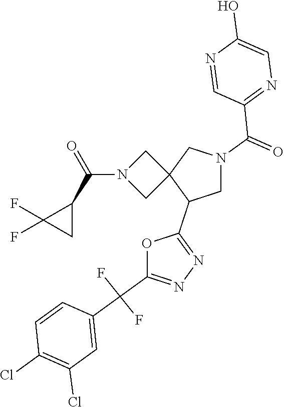 [SMILES: O=C(c1cnc(O)cn1)N1CC(c2nnc(C(F)(F)c3ccc(Cl)c(Cl)c3)o2)C2(C1)CN(C(=O)[C@H]1CC1(F)F)C2] |
| I-169 | 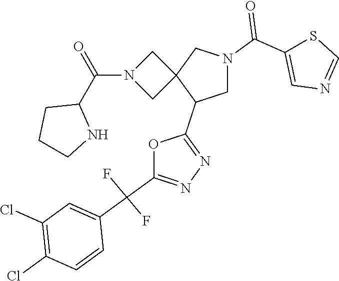 [SMILES: O=C(c1cncs1)N1CC(c2nnc(C(F)(F)c3ccc(Cl)c(Cl)c3)o2)C2(C1)CN(C(=O)C1CCCN1)C2] |
| | (A) |
| | 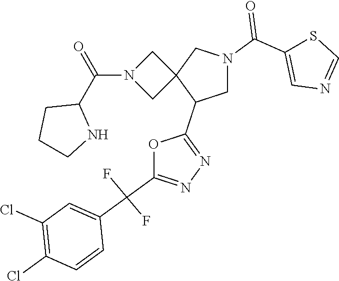 [SMILES: O=C(c1cncs1)N1CC(c2nnc(C(F)(F)c3ccc(Cl)c(Cl)c3)o2)C2(C1)CN(C(=O)C1CCCN1)C2] |
| | (B) |
| I-170 | 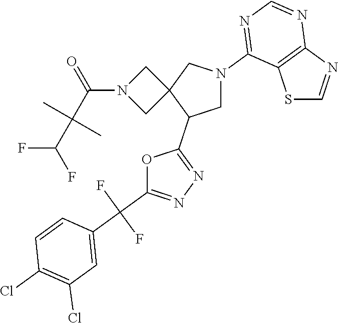 [SMILES: CC(C)(C(=O)N1CC2(C1)CN(c1ncnc3ncsc13)CC2c1nnc(C(F)(F)c2ccc(Cl)c(Cl)c2)o1)C(F)F] |
| I-171 | 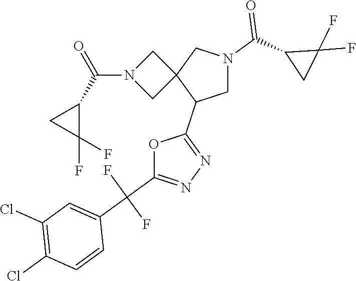 [SMILES: O=C([C@H]1CC1(F)F)N1CC(c2nnc(C(F)(F)c3ccc(Cl)c(Cl)c3)o2)C2(C1)CN(C(=O)[C@H]1CC1(F)F)C2] |
| I-172 | 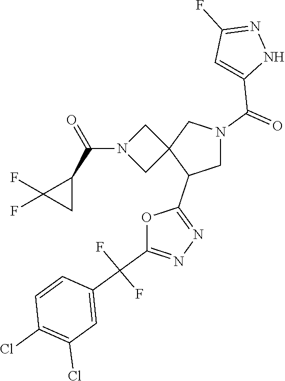 [SMILES: O=C(c1cc(F)n[nH]1)N1CC(c2nnc(C(F)(F)c3ccc(Cl)c(Cl)c3)o2)C2(C1)CN(C(=O)[C@H]1CC1(F)F)C2] |
| | (A) |
| | 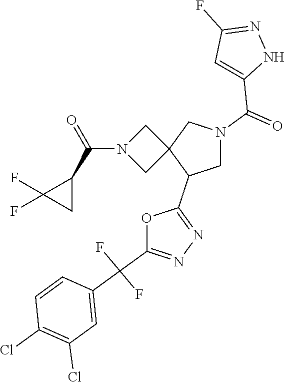 [SMILES: O=C(c1cc(F)n[nH]1)N1CC(c2nnc(C(F)(F)c3ccc(Cl)c(Cl)c3)o2)C2(C1)CN(C(=O)[C@H]1CC1(F)F)C2] |
| | (B) |
| I-173 | 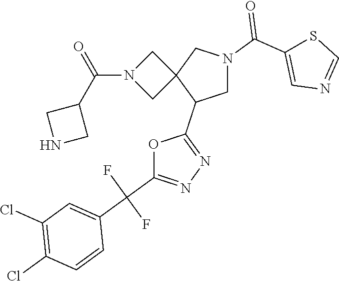 [SMILES: O=C(c1cncs1)N1CC(c2nnc(C(F)(F)c3ccc(Cl)c(Cl)c3)o2)C2(C1)CN(C(=O)C1CNC1)C2] |
| I-174 | 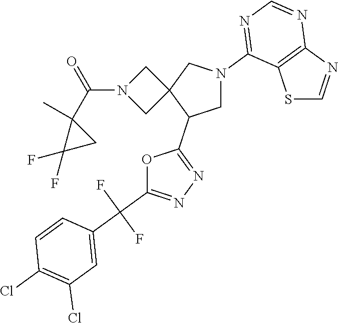 [SMILES: CC1(C(=O)N2CC3(C2)CN(c2ncnc4ncsc24)CC3c2nnc(C(F)(F)c3ccc(Cl)c(Cl)c3)o2)CC1(F)F] |
| I-175 | 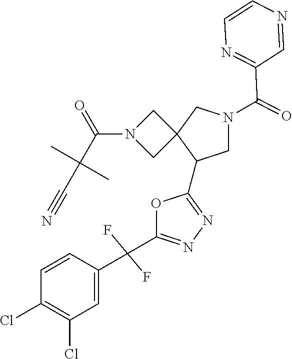 [SMILES: CC(C)(C#N)C(=O)N1CC2(CN(C(=O)c3cnccn3)CC2c2nnc(C(F)(F)c3ccc(Cl)c(Cl)c3)o2)C1] |
| I-176 | 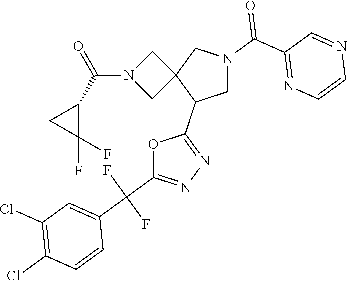 [SMILES: O=C(c1cnccn1)N1CC(c2nnc(C(F)(F)c3ccc(Cl)c(Cl)c3)o2)C2(C1)CN(C(=O)[C@H]1CC1(F)F)C2] |
| I-177 | 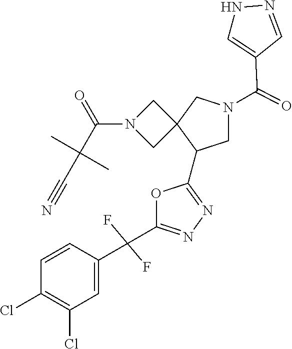 [SMILES: CC(C)(C#N)C(=O)N1CC2(CN(C(=O)c3cn[nH]c3)CC2c2nnc(C(F)(F)c3ccc(Cl)c(Cl)c3)o2)C1] |
| I-178 | 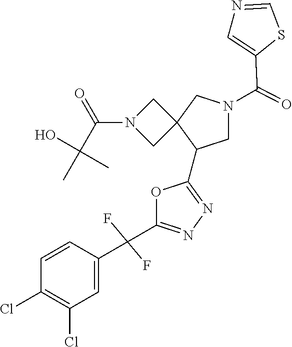 [SMILES: CC(C)(O)C(=O)N1CC2(CN(C(=O)c3cncs3)CC2c2nnc(C(F)(F)c3ccc(Cl)c(Cl)c3)o2)C1] |
| I-179 | 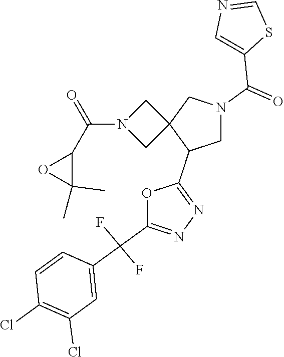 [SMILES: CC1(C)OC1C(=O)N1CC2(CN(C(=O)c3cncs3)CC2c2nnc(C(F)(F)c3ccc(Cl)c(Cl)c3)o2)C1] |
| I-180 | 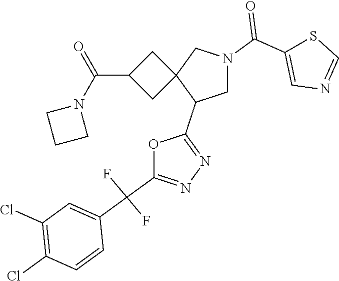 [SMILES: O=C(c1cncs1)N1CC(c2nnc(C(F)(F)c3ccc(Cl)c(Cl)c3)o2)C2(CC(C(=O)N3CCC3)C2)C1] |
| I-181 | 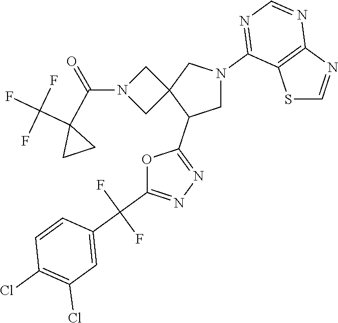 [SMILES: O=C(N1CC2(C1)CN(c1ncnc3ncsc13)CC2c1nnc(C(F)(F)c2ccc(Cl)c(Cl)c2)o1)C1(C(F)(F)F)CC1] |
| I-182 | 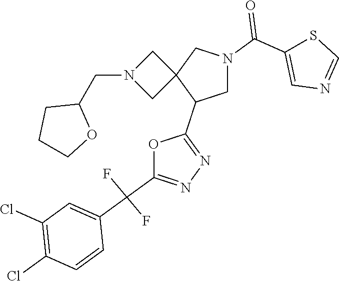 [SMILES: O=C(c1cncs1)N1CC(c2nnc(C(F)(F)c3ccc(Cl)c(Cl)c3)o2)C2(CN(CC3CCCO3)C2)C1] |
| I-183 | 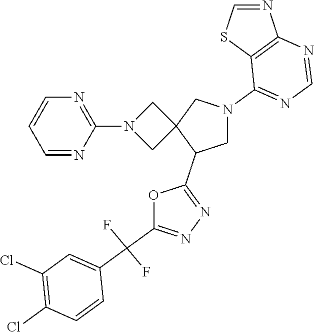 [SMILES: FC(F)(c1ccc(Cl)c(Cl)c1)c1nnc(C2CN(c3ncnc4ncsc34)CC23CN(c2ncccn2)C3)o1] |
| I-184 | 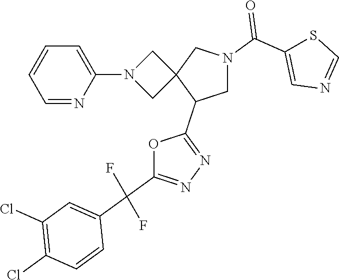 [SMILES: O=C(c1cncs1)N1CC(c2nnc(C(F)(F)c3ccc(Cl)c(Cl)c3)o2)C2(C1)CN(c1ccccn1)C2] |
| I-185 | 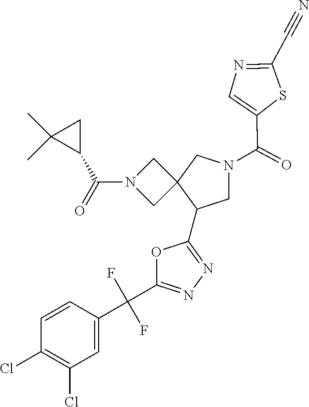 [SMILES: CC1(C)C[C@@H]1C(=O)N1CC2(CN(C(=O)c3cnc(C#N)s3)CC2c2nnc(C(F)(F)c3ccc(Cl)c(Cl)c3)o2)C1] |
| I-186 | 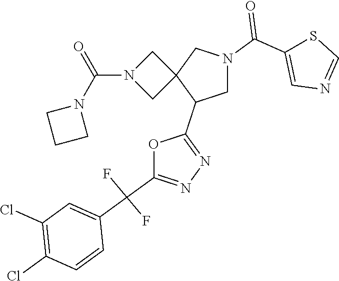 [SMILES: O=C(c1cncs1)N1CC(c2nnc(C(F)(F)c3ccc(Cl)c(Cl)c3)o2)C2(C1)CN(C(=O)N1CCC1)C2] |
| I-187 | 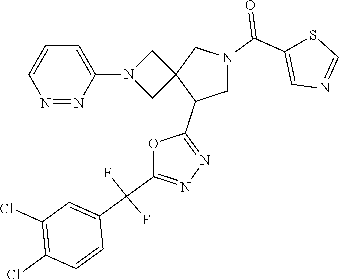 [SMILES: O=C(c1cncs1)N1CC(c2nnc(C(F)(F)c3ccc(Cl)c(Cl)c3)o2)C2(C1)CN(c1cccnn1)C2] |
| I-188 | 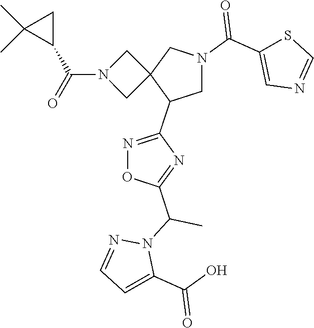 [SMILES: CC(c1nc(C2CN(C(=O)c3cncs3)CC23CN(C(=O)[C@H]2CC2(C)C)C3)no1)n1nccc1C(=O)O] |
| I-189 | 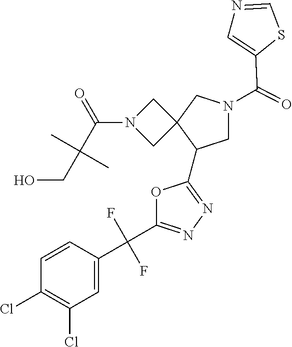 [SMILES: CC(C)(CO)C(=O)N1CC2(CN(C(=O)c3cncs3)CC2c2nnc(C(F)(F)c3ccc(Cl)c(Cl)c3)o2)C1] |
| I-190 | 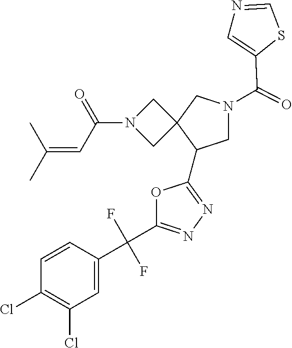 [SMILES: CC(C)=CC(=O)N1CC2(C1)CN(C(=O)c1cncs1)CC2c1nnc(C(F)(F)c2ccc(Cl)c(Cl)c2)o1] |
| I-191 | 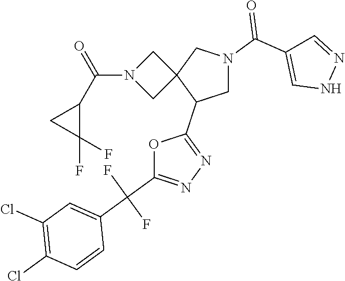 [SMILES: O=C(c1cn[nH]c1)N1CC(c2nnc(C(F)(F)c3ccc(Cl)c(Cl)c3)o2)C2(C1)CN(C(=O)C1CC1(F)F)C2] |
| I-192 | 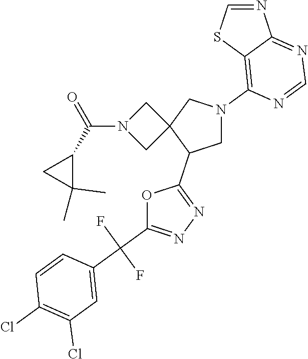 [SMILES: CC1(C)C[C@@H]1C(=O)N1CC2(C1)CN(c1ncnc3ncsc13)CC2c1nnc(C(F)(F)c2ccc(Cl)c(Cl)c2)o1] |
| I-193 | 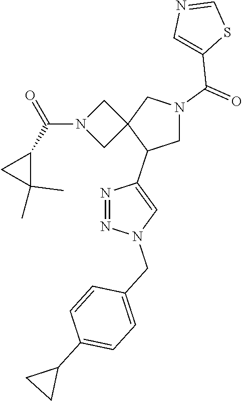 [SMILES: CC1(C)C[C@@H]1C(=O)N1CC2(CN(C(=O)c3cncs3)CC2c2cn(Cc3ccc(C4CC4)cc3)nn2)C1] |
| I-194 | 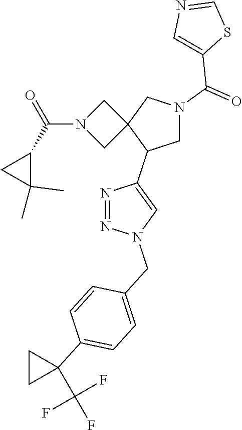 [SMILES: CC1(C)C[C@@H]1C(=O)N1CC2(CN(C(=O)c3cncs3)CC2c2cn(Cc3ccc(C4(C(F)(F)F)CC4)cc3)nn2)C1] |
| I-195 | 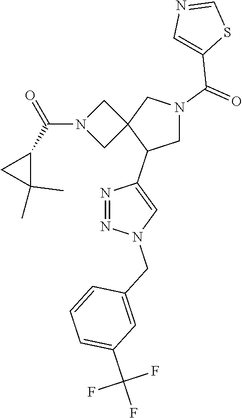 [SMILES: CC1(C)C[C@@H]1C(=O)N1CC2(CN(C(=O)c3cncs3)CC2c2cn(Cc3cccc(C(F)(F)F)c3)nn2)C1] |
| I-196 | 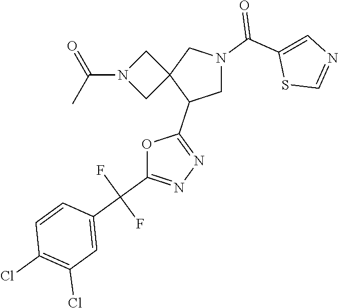 [SMILES: CC(=O)N1CC2(C1)CN(C(=O)c1cncs1)CC2c1nnc(C(F)(F)c2ccc(Cl)c(Cl)c2)o1] |
| I-197 | 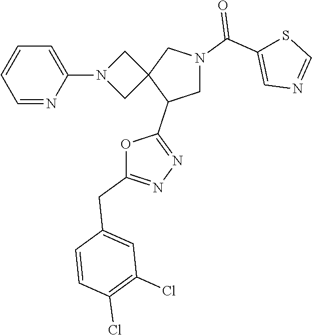 [SMILES: O=C(c1cncs1)N1CC(c2nnc(Cc3ccc(Cl)c(Cl)c3)o2)C2(C1)CN(c1ccccn1)C2] |
| I-198 | 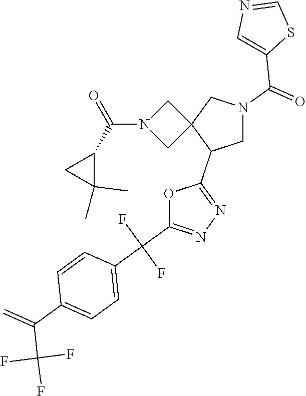 [SMILES: C=C(c1ccc(C(F)(F)c2nnc(C3CN(C(=O)c4cncs4)CC34CN(C(=O)[C@H]3CC3(C)C)C4)o2)cc1)C(F)(F)F] |
| I-199 | 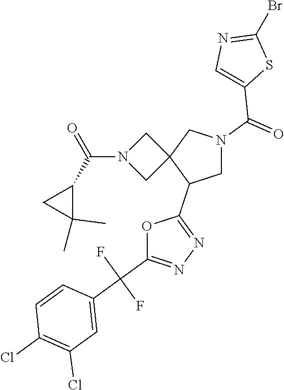 [SMILES: CC1(C)C[C@@H]1C(=O)N1CC2(CN(C(=O)c3cnc(Br)s3)CC2c2nnc(C(F)(F)c3ccc(Cl)c(Cl)c3)o2)C1] |
| I-200 | 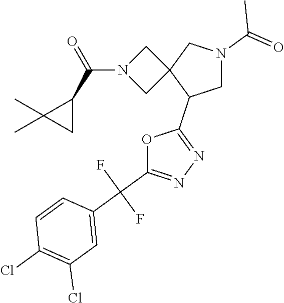 [SMILES: CC(=O)N1CC(c2nnc(C(F)(F)c3ccc(Cl)c(Cl)c3)o2)C2(C1)CN(C(=O)[C@H]1CC1(C)C)C2] |
| I-201 | 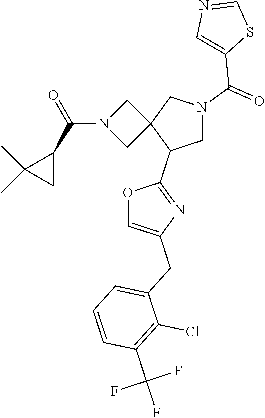 [SMILES: CC1(C)C[C@@H]1C(=O)N1CC2(CN(C(=O)c3cncs3)CC2c2nc(Cc3cccc(C(F)(F)F)c3Cl)co2)C1] |
| I-202 | 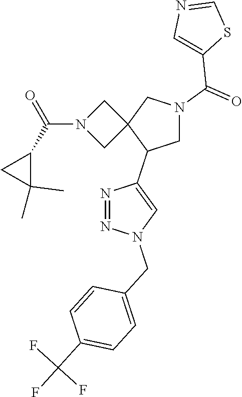 [SMILES: CC1(C)C[C@@H]1C(=O)N1CC2(CN(C(=O)c3cncs3)CC2c2cn(Cc3ccc(C(F)(F)F)cc3)nn2)C1] |
| I-203 | 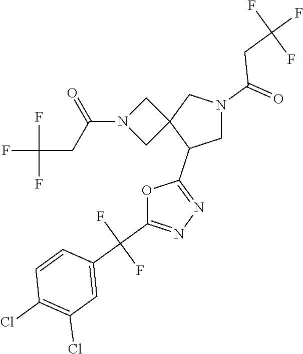 [SMILES: O=C(CC(F)(F)F)N1CC(c2nnc(C(F)(F)c3ccc(Cl)c(Cl)c3)o2)C2(C1)CN(C(=O)CC(F)(F)F)C2] |
| I-204 | 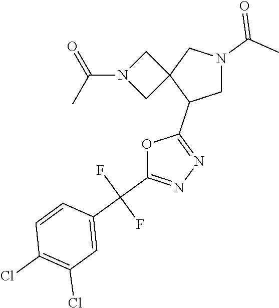 [SMILES: CC(=O)N1CC(c2nnc(C(F)(F)c3ccc(Cl)c(Cl)c3)o2)C2(C1)CN(C(C)=O)C2] |
| I-205 | 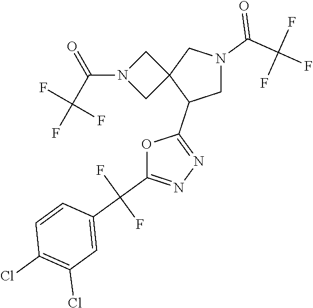 [SMILES: O=C(N1CC(c2nnc(C(F)(F)c3ccc(Cl)c(Cl)c3)o2)C2(C1)CN(C(=O)C(F)(F)F)C2)C(F)(F)F] |
| I-206 | 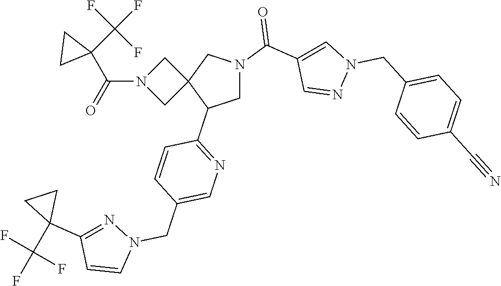 [SMILES: N#Cc1ccc(Cn2cc(C(=O)N3CC(c4ccc(Cn5ccc(C6(C(F)(F)F)CC6)n5)cn4)C4(C3)CN(C(=O)C3(C(F)(F)F)CC3)C4)cn2)cc1] |
| I-207 | 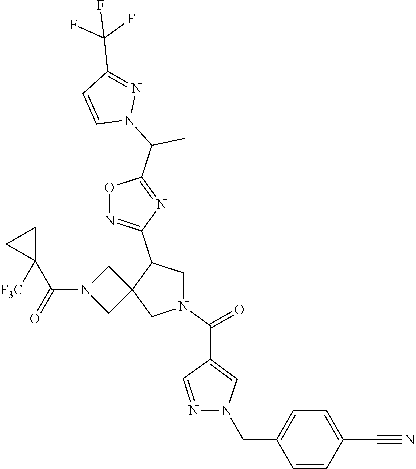 [SMILES: CC(c1nc(C2CN(C(=O)c3cnn(Cc4ccc(C#N)cc4)c3)CC23CN(C(=O)C2(C(F)(F)F)CC2)C3)no1)n1ccc(C(F)(F)F)n1] |
| I-208 | 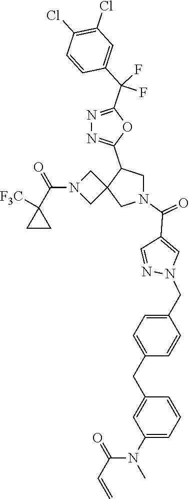 [SMILES: C=CC(=O)N(C)c1cccc(Cc2ccc(Cn3cc(C(=O)N4CC(c5nnc(C(F)(F)c6ccc(Cl)c(Cl)c6)o5)C5(C4)CN(C(=O)C4(C(F)(F)F)CC4)C5)cn3)cc2)c1] |
| I-213 | 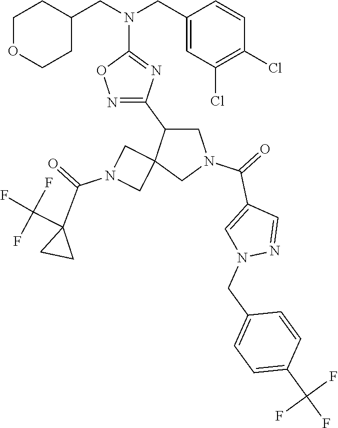 [SMILES: O=C(c1cnn(Cc2ccc(C(F)(F)F)cc2)c1)N1CC(c2noc(N(Cc3ccc(Cl)c(Cl)c3)CC3CCOCC3)n2)C2(C1)CN(C(=O)C1(C(F)(F)F)CC1)C2] |
| I-214 | 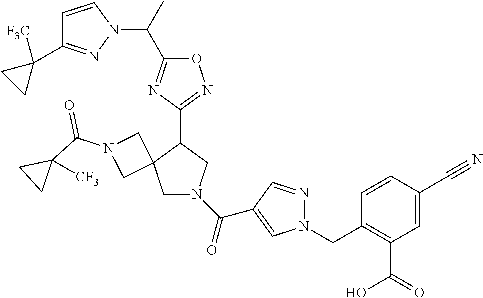 [SMILES: CC(c1nc(C2CN(C(=O)c3cnn(Cc4ccc(C#N)cc4C(=O)O)c3)CC23CN(C(=O)C2(C(F)(F)F)CC2)C3)no1)n1ccc(C2(C(F)(F)F)CC2)n1] |
| I-215 | 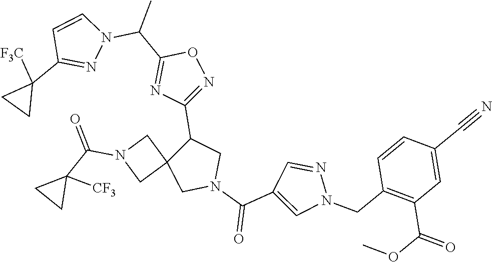 [SMILES: COC(=O)c1cc(C#N)ccc1Cn1cc(C(=O)N2CC(c3noc(C(C)n4ccc(C5(C(F)(F)F)CC5)n4)n3)C3(C2)CN(C(=O)C2(C(F)(F)F)CC2)C3)cn1] |
| I-216 | 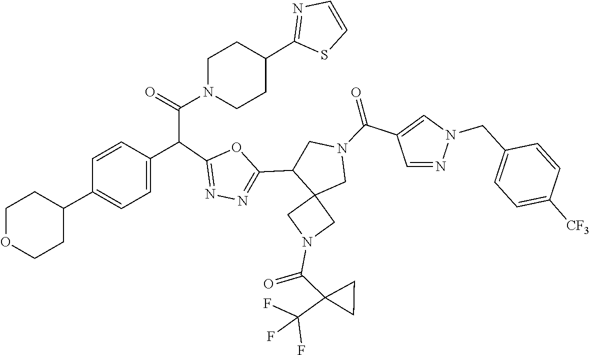 [SMILES: O=C(c1cnn(Cc2ccc(C(F)(F)F)cc2)c1)N1CC(c2nnc(C(C(=O)N3CCC(c4nccs4)CC3)c3ccc(C4CCOCC4)cc3)o2)C2(C1)CN(C(=O)C1(C(F)(F)F)CC1)C2] |
| I-217 | 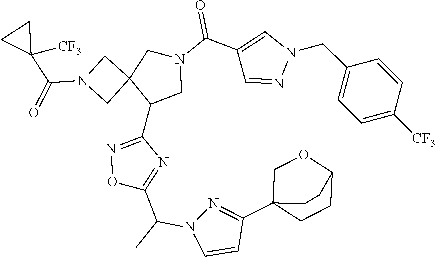 [SMILES: CC(c1nc(C2CN(C(=O)c3cnn(Cc4ccc(C(F)(F)F)cc4)c3)CC23CN(C(=O)C2(C(F)(F)F)CC2)C3)no1)n1ccc(C23CCC(CC2)OC3)n1] |
| I-218 | 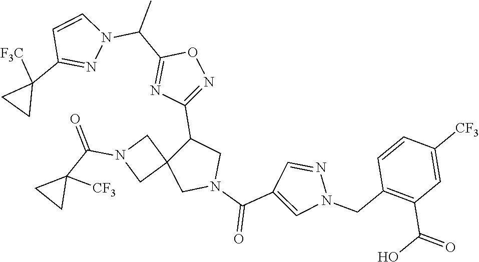 [SMILES: CC(c1nc(C2CN(C(=O)c3cnn(Cc4ccc(C(F)(F)F)cc4C(=O)O)c3)CC23CN(C(=O)C2(C(F)(F)F)CC2)C3)no1)n1ccc(C2(C(F)(F)F)CC2)n1] |
| I-219 | 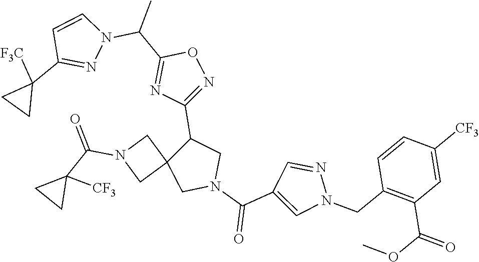 [SMILES: COC(=O)c1cc(C(F)(F)F)ccc1Cn1cc(C(=O)N2CC(c3noc(C(C)n4ccc(C5(C(F)(F)F)CC5)n4)n3)C3(C2)CN(C(=O)C2(C(F)(F)F)CC2)C3)cn1] |
| I-220 | 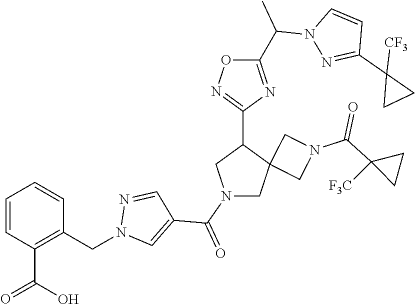 [SMILES: CC(c1nc(C2CN(C(=O)c3cnn(Cc4ccccc4C(=O)O)c3)CC23CN(C(=O)C2(C(F)(F)F)CC2)C3)no1)n1ccc(C2(C(F)(F)F)CC2)n1] |
| I-221 | 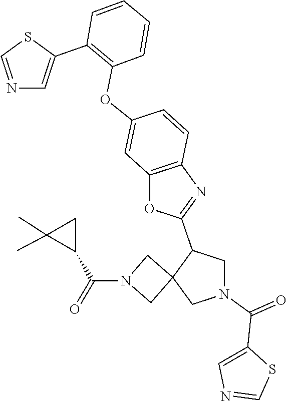 [SMILES: CC1(C)C[C@@H]1C(=O)N1CC2(CN(C(=O)c3cncs3)CC2c2nc3ccc(Oc4ccccc4-c4cncs4)cc3o2)C1] |
| I-222 | 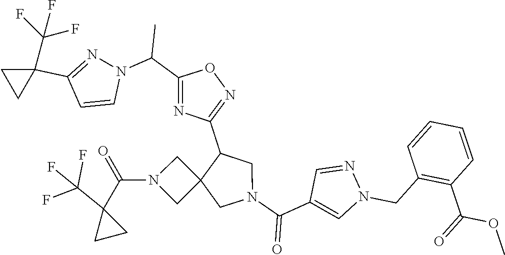 [SMILES: COC(=O)c1ccccc1Cn1cc(C(=O)N2CC(c3noc(C(C)n4ccc(C5(C(F)(F)F)CC5)n4)n3)C3(C2)CN(C(=O)C2(C(F)(F)F)CC2)C3)cn1] |
| I-223 | 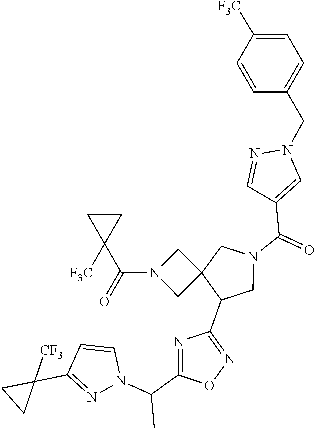 [SMILES: CC(c1nc(C2CN(C(=O)c3cnn(Cc4ccc(C(F)(F)F)cc4)c3)CC23CN(C(=O)C2(C(F)(F)F)CC2)C3)no1)n1ccc(C2(C(F)(F)F)CC2)n1] |
| I-209 | 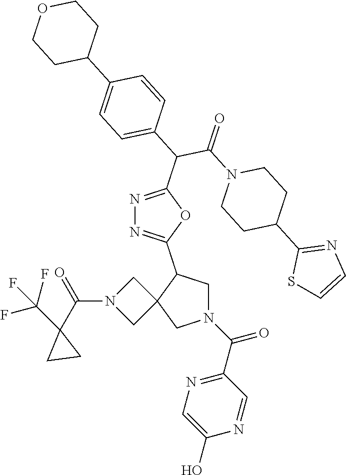 [SMILES: O=C(c1cnc(O)cn1)N1CC(c2nnc(C(C(=O)N3CCC(c4nccs4)CC3)c3ccc(C4CCOCC4)cc3)o2)C2(C1)CN(C(=O)C1(C(F)(F)F)CC1)C2] |
| I-210 | 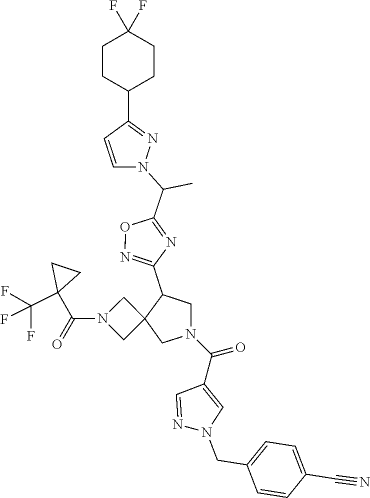 [SMILES: CC(c1nc(C2CN(C(=O)c3cnn(Cc4ccc(C#N)cc4)c3)CC23CN(C(=O)C2(C(F)(F)F)CC2)C3)no1)n1ccc(C2CCC(F)(F)CC2)n1] |
| I-211 | 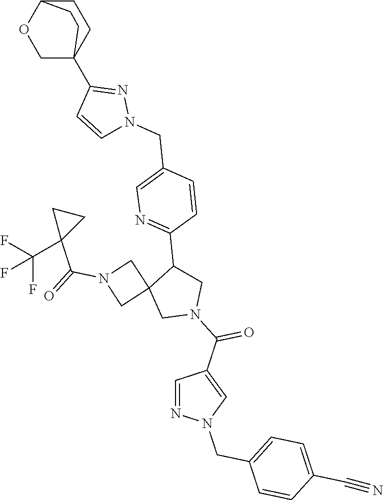 [SMILES: N#Cc1ccc(Cn2cc(C(=O)N3CC(c4ccc(Cn5ccc(C67CCC(CC6)OC7)n5)cn4)C4(C3)CN(C(=O)C3(C(F)(F)F)CC3)C4)cn2)cc1] |
| I-212 | 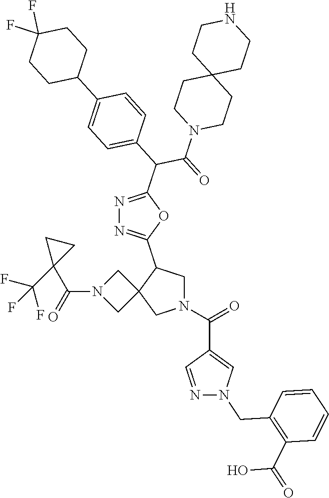 [SMILES: O=C(O)c1ccccc1Cn1cc(C(=O)N2CC(c3nnc(C(C(=O)N4CCC5(CCNCC5)CC4)c4ccc(C5CCC(F)(F)CC5)cc4)o3)C3(C2)CN(C(=O)C2(C(F)(F)F)CC2)C3)cn1] |
| I-224 | 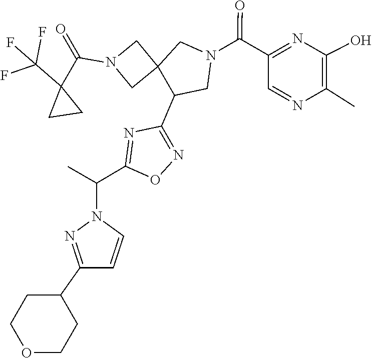 [SMILES: Cc1ncc(C(=O)N2CC(c3noc(C(C)n4ccc(C5CCOCC5)n4)n3)C3(C2)CN(C(=O)C2(C(F)(F)F)CC2)C3)nc1O] |
| I-225 | 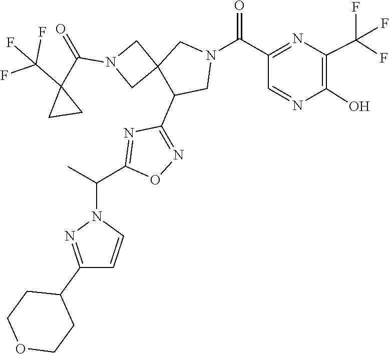 [SMILES: CC(c1nc(C2CN(C(=O)c3cnc(O)c(C(F)(F)F)n3)CC23CN(C(=O)C2(C(F)(F)F)CC2)C3)no1)n1ccc(C2CCOCC2)n1] |
| I-226 | 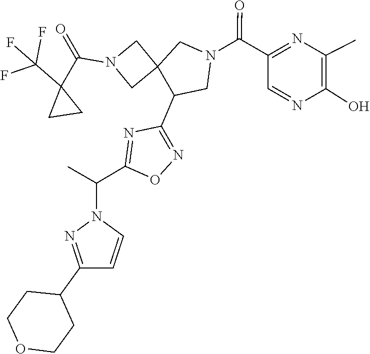 [SMILES: Cc1nc(C(=O)N2CC(c3noc(C(C)n4ccc(C5CCOCC5)n4)n3)C3(C2)CN(C(=O)C2(C(F)(F)F)CC2)C3)cnc1O] |
| I-230 | 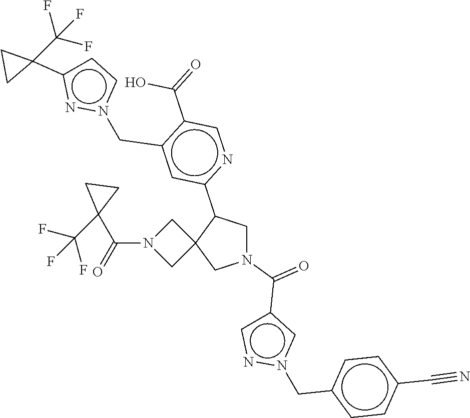 |
| I-231 | 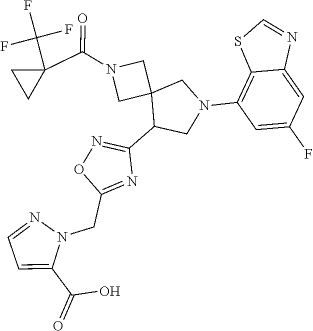 [SMILES: O=C(O)c1ccnn1Cc1nc(C2CN(c3cc(F)cc4ncsc34)CC23CN(C(=O)C2(C(F)(F)F)CC2)C3)no1] |
| I-232 | 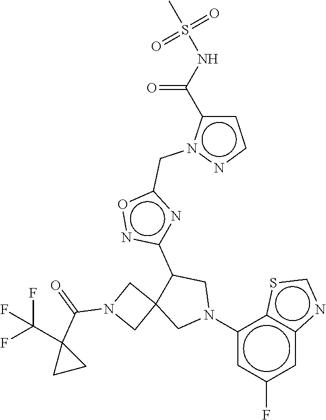 |
| I-233 | 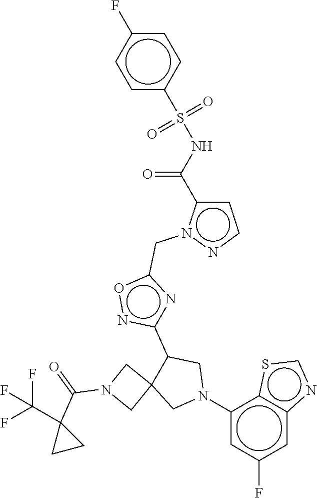 |
| I-234 | 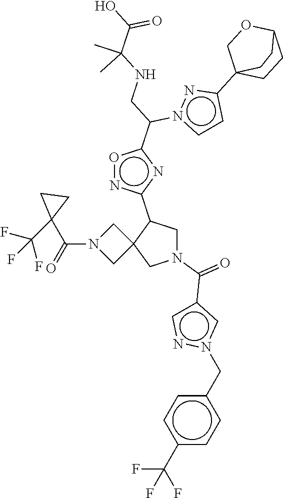 |
| I-235 | 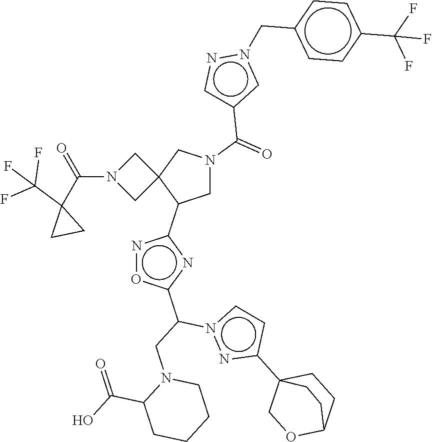 |
| I-236 | 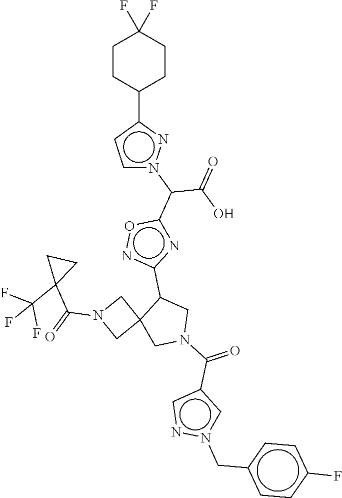 |
| I-237 | 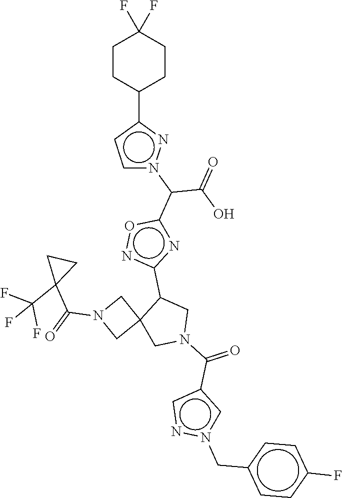 |
| I-238 | 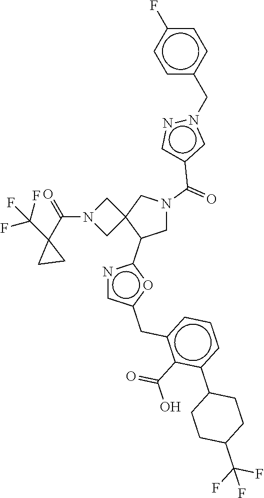 |
| I-239 | 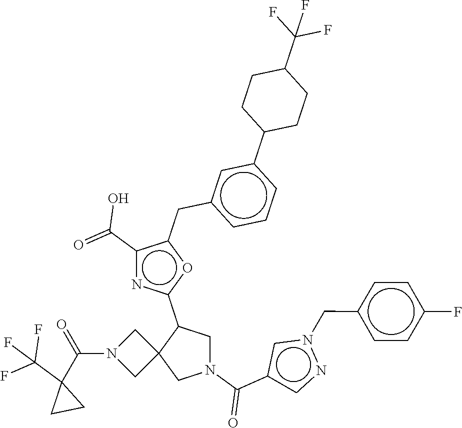 |
| I-240 | 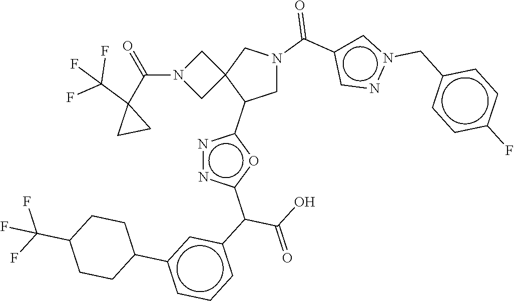 |
| I-241 | 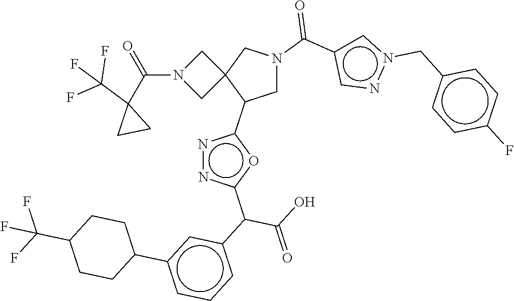 |
| I-242 | 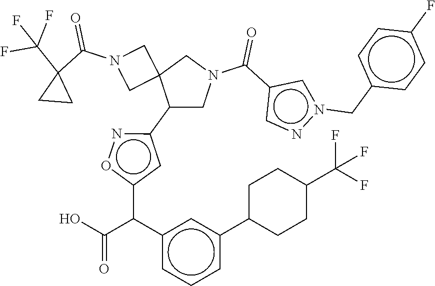 |
| I-243 | 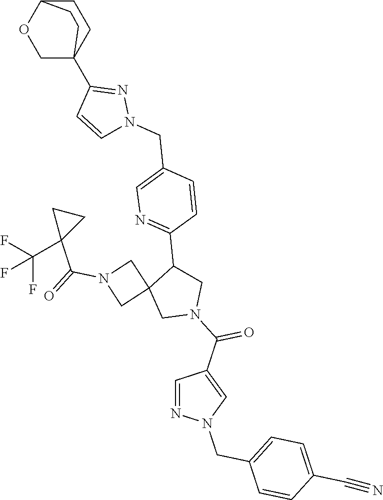 [SMILES: N#Cc1ccc(Cn2cc(C(=O)N3CC(c4ccc(Cn5ccc(C67CCC(CC6)OC7)n5)cn4)C4(C3)CN(C(=O)C3(C(F)(F)F)CC3)C4)cn2)cc1] |

In some embodiments, the present disclosure provides a compound set forth in Table 8, above, or a pharmaceutically acceptable salt thereof. In some embodiments, the disclosure provides a compound set forth in Table 8, above, or a pharmaceutically acceptable salt thereof, and any enantiomers, diastereomers, or conformation isomers thereof.

In some embodiments, the present disclosure provides a pharmaceutical composition comprising a compound of the present disclosure, or a pharmaceutically acceptable salt thereof, together with a pharmaceutically acceptable carrier, excipient, vehicle, adjuvant or diluent. In some embodiments, the present disclosure provides a pharmaceutical composition comprising a compound set forth in Table 8 above, or a pharmaceutically acceptable salt thereof, together with a pharmaceutically acceptable carrier, excipient, vehicle, adjuvant or diluent. In some embodiments, the pharmaceutical composition further comprises an additional therapeutic agent.

In some embodiments, the present disclosure provides a complex comprising a CDK2 protein and a compound of the present disclosure.

In some embodiments, the present disclosure provides a method of inhibiting the activity of a cyclin-dependent kinase (CDK). In some embodiments, the method comprises contacting a compound of the present disclosure with a CDK. In some embodiments, the compound and the CDK are contacted in vivo. In some embodiments, the compound and the CDK are contacted in vitro. In some embodiments, the CDK is selected from CDK1, CDK2, CDK3, CDK4, CDK5, CDK6, CDK7, CDK8, CDK9, CDK10, CDK11, CDK12 and CDK13. In some embodiments, the CDK is CDK2. In some embodiments, the CDK is CDK3. In some embodiments, the CDK is CDK4. In some embodiments, the CDK is CDK6. In some embodiments, the method inhibits the activity of both CDK2 and CDK3. In some embodiments, the method inhibits the activity of CDK2 and one or both of CDK4 and CDK6.

In some embodiments, the compounds of the present disclosure inhibit the activity of one or more CDKs selected from CDK1, CDK2, CDK3, CDK4, CDK5, CDK6, CDK7, CDK8, CDK9, CDK10, CDK11, CDK12 and CDK13. In some embodiments, the compounds of the present disclosure inhibit CDK2. In some embodiments, the compounds of the present disclosure inhibit CDK3. In some embodiments, the compounds of the present disclosure inhibit CDK4. In some embodiments, the compounds of the present disclosure inhibit CDK6. In some embodiments, the compounds of the present disclosure are CDK2/3 inhibitors. In some embodiments, the compounds of the present disclosure are CDK2/4/6 inhibitors.

In some embodiments, the present disclosure provides compounds that selectively inhibit CDK2 over other cyclin-dependent kinases (CDKs). In some embodiments, the compounds of the present disclosure selectively inhibit CDK2 over one or more other CDKs, selected from CDK1, CDK3, CDK4, CDK5, CDK6, CDK7, CDK8, CDK9, CDK10, CDK11, CDK12 and CDK13. In some embodiments, the compounds of the present disclosure selectively inhibit CDK2 over CDK4. In some embodiments, the compounds of the present disclosure selectively inhibit CDK2 over CDK6. In some embodiments, the compounds of the present disclosure selectively inhibit CDK2 over CDK4 and CDK6.

In some embodiments, the present disclosure provides compounds that selectively inhibit CDK2/cyclin E complexes over other CDK complexes.

### 4. General Methods of Providing the Present Compounds

The compounds of this disclosure may be prepared or isolated in general by synthetic and/or semi-synthetic methods known to those skilled in the art for analogous compounds and by methods described in detail in the Examples, herein.

In the Schemes below, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “leaving group” (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

Compounds of the present disclosure, including those of Formula I and the compounds of Table 8, can generally be prepared according the methods described below. Reagents and conditions can be modified and substituted using knowledge common to one of ordinary skill in the art, as needed, in order to arrive at the compounds of the present disclosure.

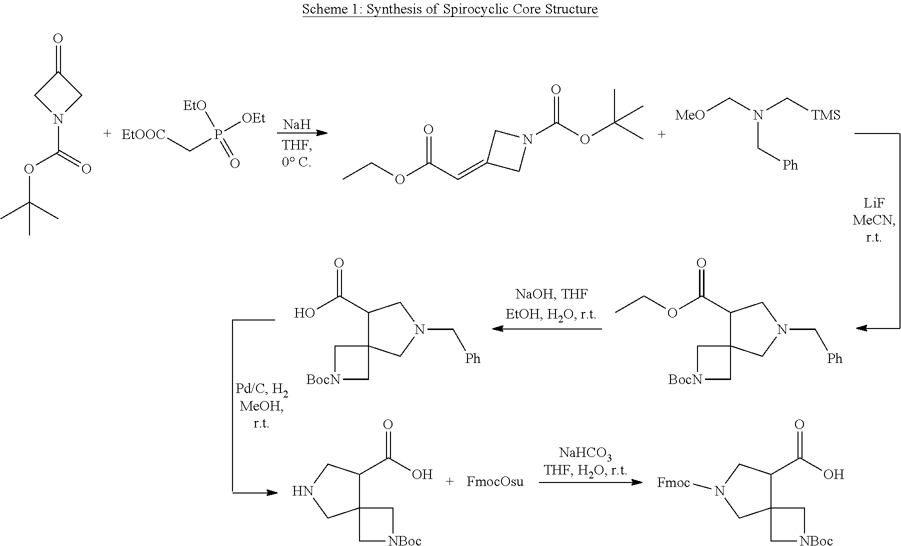

### 5. Uses, Formulation and Administration

### Pharmaceutically Acceptable Compositions

According to another embodiment, the disclosure provides a composition comprising a compound of this disclosure or a pharmaceutically acceptable derivative thereof and a pharmaceutically acceptable carrier, adjuvant, or vehicle. The amount of compound in compositions of this disclosure is such that it is effective to measurably inhibit a CDK2 protein, or a mutant thereof, in a biological sample or in a patient. In certain embodiments, the amount of compound in compositions of this disclosure is such that it is effective to measurably inhibit a CDK2 protein, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered subcutaneously, orally, intraperitoneally or intravenously. In some embodiments, the compositions are administered orally. In some embodiments, the compositions are administered intraperitoneally. In some embodiments, the compositions are administered intravenously. In some embodiments, the compositions are administered subcutaneously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of Compounds and Pharmaceutically Acceptable Compositions

Compounds and compositions described herein are generally useful for the modulation of the activity CDK2. In some embodiments, the compounds and compositions described herein are CDK2 inhibitors.

In some embodiments, the compounds and compositions of the present disclosure are useful for treating diseases and disorders associated with CDK2 activity, including, but not limited to cancers, myeloproliferative disorders, autoimmune disorders, inflammatory disorders, viral infections, fibrotic disorders, and neurodegenerative disorders.

In some embodiments, the disclosure provides a method of inhibiting the activity of a CDK2, the method comprising contacting a compound of the present disclosure, or a pharmaceutically acceptable salt thereof with the CDK2. In some embodiments, the contacting takes place in vitro. In some embodiments, the contacting takes place in vivo.

In some embodiments, the disclosure provides a method of treating, preventing or lessening the severity of a disease or disorder associated with CDK2 activity in a patient, including, but not limited to cancers, myeloproliferative disorders, autoimmune disorders, inflammatory disorders, fibrotic disorders, and neurodegenerative disorders, said method comprising administering to a patient in need thereof, a compound of the present disclosure, or a pharmaceutically acceptable salt thereof, or a pharmaceutical composition comprising an effective amount of a compound of the present disclosure, or a pharmaceutically acceptable salt thereof.

The disclosure further provides a compound of the present disclosure, or a pharmaceutically acceptable salt thereof, or a pharmaceutical composition comprising an effective amount of a compound of the present disclosure, or a pharmaceutically acceptable salt thereof, for use in the treatment of a disease or disorder associated with CDK2 activity.

The disclosure further provides a compound of the present disclosure, or a pharmaceutically acceptable salt thereof, or a pharmaceutical composition comprising an effective amount of a compound of the present disclosure, or a pharmaceutically acceptable salt thereof, for use in the manufacture of a medicament for treating a disease or disorder associated with CDK2 activity.

In some embodiments, the disease or disorder associated with CDK2 activity is a CDK2-mediated disease or disorder. In some embodiments, the disease or disorder associated with CDK2 activity is a disease or disorder caused by CDK2 over-activity.

In some embodiments, the disease or disorder associated with CDK2 activity is cancer.

In some embodiments, the cancer is selected from breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, melanoma and thyroid cancer.

In some embodiments, the cancer is characterized by amplification or overexpression of CCNE1 and/or CCNE2.

In some embodiments, the cancer is breast cancer. In some embodiments, the breast cancer is a breast cancer selected from ER-positive/HR-positive breast cancer, HER2-negative breast cancer, ER-positive/HR-positive breast cancer, HER2-positive breast cancer, triple negative breast cancer (TNBC), inflammatory breast cancer, endocrine resistant breast cancer, trastuzumab resistant breast cancer, breast cancer with primary or acquired resistance to CDK4/CDK6 inhibition, advanced breast cancer and metastatic breast cancer. In some embodiments the breast cancer is characterized by amplification or overexpression of CCNE1 and/or CCNE2.

In some embodiments, the cancer is ovarian cancer. In some embodiments, the ovarian cancer is high-grade serous ovarian cancer (HGSOC). In some embodiments the ovarian cancer is characterized by amplification or overexpression of CCNE1 and/or CCNE2.

In some embodiments, the cancer is bladder cancer. In some embodiments, the bladder cancer is characterized by amplification or overexpression of CCNE1 and/or CCNE2.

In some embodiments, the cancer is uterine cancer. In some embodiments, the uterine cancer is characterized by amplification or overexpression of CCNE1 and/or CCNE2.

In some embodiments, the cancer is prostate cancer. In some embodiments, the prostate cancer is characterized by amplification or overexpression of CCNE1 and/or CCNE2.

In some embodiments, the cancer is lung cancer. In some embodiments, the lung cancer is a lung cancer selected from non-small cell lung cancer, small cell lung cancer, squamous cell carcinoma, adenocarcinoma, and mesothelioma. In some embodiments, the lung cancer is characterized by amplification or overexpression of CCNE1 and/or CCNE2. In some embodiments, the lung cancer is CCNE1 amplified squamous cell carcinoma or CCNE1 amplified adenocarcinoma.

In some embodiments, the cancer is head and neck cancer. In some embodiments, the head and neck cancer is characterized by amplification or overexpression of CCNE1 and/or CCNE2.

In some embodiments, the cancer is colorectal cancer. In some embodiments, the colorectal cancer is characterized by amplification or overexpression of CCNE1 and/or CCNE2.

In some embodiments, the cancer is kidney cancer. In some embodiments, the kidney cancer is renal cell carcinoma (RCC). In some embodiments, the kidney cancer is characterized by amplification or overexpression of CCNE1 and/or CCNE2.

In some embodiments, the cancer is liver cancer. In some embodiments, the liver cancer is hepatocellular carcinoma (HCC). In some embodiments, the liver cancer is characterized by amplification or overexpression of CCNE1 and/or CCNE2.

In some embodiments, the cancer is pancreatic cancer. In some embodiments, the pancreatic cancer is characterized by amplification or overexpression of CCNE1 and/or CCNE2.

In some embodiments, the cancer is stomach cancer. In some embodiments, the stomach cancer is characterized by amplification or overexpression of CCNE1 and/or CCNE2.

In some embodiments, the cancer is melanoma. In some embodiments, the melanoma is characterized by amplification or overexpression of CCNE1 and/or CCNE2. CDK2 expression is regulated by essential melanocytic transcription factor MITF. It has been found that CDK2 depletion suppresses the growth of melanoma (Du et al., Cancer Cell. 2004 December; 6(6): 565-576).

In some embodiments, the cancer is thyroid cancer. In some embodiments, the thyroid cancer is characterized by amplification or overexpression of CCNE1 and/or CCNE2.

In some embodiments, the disease or disorder associated with CDK2 activity is a myeloproliferative disorder.

In some embodiments, the disease or disorder associated with CDK2 activity is a neurodegenerative disease or disorder. In some embodiments, the neurodegenerative disease or disorder is Alzheimer's disease (AD). It has been reported that neuronal cell death in subjects suffering from AD is preceded by cell cycle events. Inhibition of one or more CDKs can inhibit cell cycle events and therefore stave off neuronal cell death (Yang et al., J Neurosci. 2003 Apr. 1; 23(7):2557-2563).

In some embodiments, the disease or disorder associated with CDK2 activity is a liver disease.

In some embodiments, the disease or disorder associated with CDK2 activity is liver fibrosis. It has been reported that CCNE1 knockout mice do not develop liver fibrosis upon exposure to pro-fibrotic toxin CCl₄, suggesting that liver fibrosis can be treated via administration of a CDK2 inhibitor (Nevzorova, et al., Hepatology. 2012 September; 56(3): 1140-1149.)

In some embodiments, the disease or disorder associated with CDK2 activity is Cushing disease. Pituitary cyclin E/E2F1 signaling is a molecular mechanism underlying neuroendocrine regulation of the hypothalamic-pituitary-adrenal axis, and therefore provides a subcellular therapeutic target for CDK2 inhibitors of pituitary ACTH-dependent hypercortisolism, also known as Cushing disease (Liu, et al., J Clin Endocrinol Metab. 2015 July; 100(7): 2557-2564.).

In some embodiments, the disease or disorder associated with CDK2 activity is a kidney disease.

In some embodiments, the disease or disorder associated with CDK2 activity is polycystic kidney disease. It has been reported that CDK2/CDK5 inhibitor roscovitine yields effective arrest of cystic kidney disease in mouse models of polycystic kidney disease (Bukanov, et al., Nature. 2006 Dec. 14; 444(7121):949-52).

In some embodiments, the disease or disorder associated with CDK2 activity is an autoimmune disorder. CDK2 ablation has been shown to promote immune tolerance by supporting the function of regulatory T cells (Chunder et al., J Immunol. 2012 Dec. 15; 189(12):5659-66).

In some embodiments, the disease or disorder associated with CDK2 activity is an inflammatory disorder. Cyclin E ablation has been shown to attenuate hepatitis in mice, while p27 knockout mice display exacerbation of renal inflammation (Ehedego et al., Oncogene. 2018 June; 37(25):3329-3339.; Ophascharoensuk et al., Nat Med. 1998 May; 4(5):575-80.). In some embodiments, the inflammatory disorder is hepatitis.

In some embodiments, the compounds and compositions of the present disclosure are useful as male contraceptives. Based on the finding that male CDK2 knockout mice are sterile, CDK2 inhibitors have been studied as possible male contraceptives (Faber, et al., Biol Reprod. 2020 August; 103(2): 357-367.). In some embodiments, the present disclosure provides a method of reducing male fertility comprising administering to a patient in need thereof, a compound of the present disclosure, or a pharmaceutically acceptable salt thereof, or a pharmaceutical composition comprising an effective amount of a compound of the present disclosure, or a pharmaceutically acceptable salt thereof.

### Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents that the compounds of the present disclosure may also be combined with include, without limitation: endocrine therapeutic agents, chemotherapeutic agents and other CDK inhibitory compounds.

Tn some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of an endocrine therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional CDK inhibitory compounds. In some embodiments, the CDK inhibitory compounds are CDK4 or CDK4/CDK6 inhibitors.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of a chemotherapeutic agent. In some embodiments, the chemotherapeutic agent is a taxane. In some embodiments, the chemotherapeutic agent is a platinum agent. In some embodiments, the chemotherapeutic agent is trastuzumab.

As used herein, the term “combination,” “combined,” and related terms refers to the simultaneous or sequential administration of therapeutic agents in accordance with this disclosure. For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the present disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the present disclosure may be administered simultaneously, sequentially or within a period of time from one another, for example within 1, 2, 3, 4, 5, 6, 7, 8, 9, 10, 11, 12, 13, 14, 15, 16, 17, 18, 19, 20, 21, 22, 23, or 24 hours from one another. In some embodiments, one or more other therapeutic agent and a compound or composition the present disclosure are administered as a multiple dosage regimen within greater than 24 hours a parts.

In one embodiment, the present disclosure provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

### EXAMPLES

As depicted in the Examples below, in certain exemplary embodiments, compounds are prepared according to the general procedures provided herein. It will be appreciated that, although the general methods depict the synthesis of certain compounds of the present disclosure, the general methods, and other methods known to one of ordinary skill in the art, can be applied to all compounds and subclasses and species of each of these compounds, as described herein.

### Example 1: Synthesis of Compounds of the Disclosure

### Synthesis of N-(3-(6-(1-benzyl-1H-pyrazole-4-carbonyl)-2-((S)-2,2-dimethylcyclopropane-1-carbonyl)-2,6-diazaspiro[3.4]octan-8-yl)isoxazol-5-yl)cyclopropanecarboxamide (I-7)

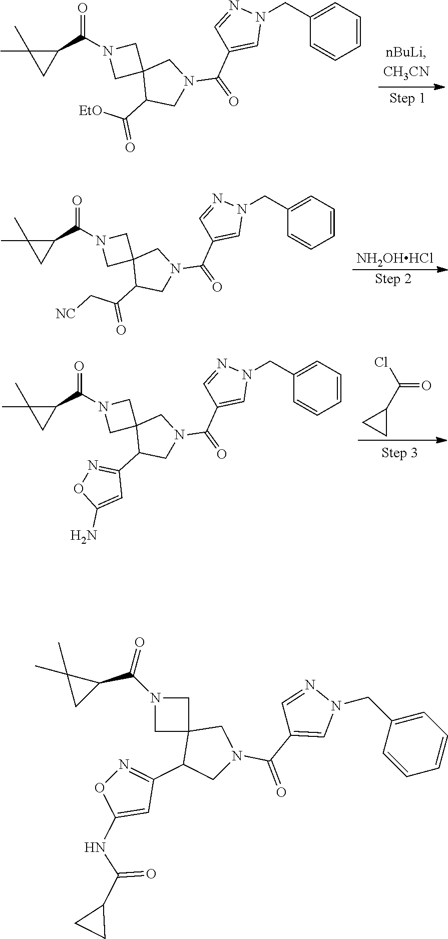

### Step 1: 3-(6-(1-benzyl-1H-pyrazole-4-carbonyl)-2-((S)-2,2-dimethylcyclopropane-1-carbonyl)-2,6-diazaspiro[3.4]octan-8-yl)-3-oxopropanenitrile

To a solution of ethyl 6-(1-benzyl-1H-pyrazole-4-carbonyl)-2-((S)-2,2-dimethylcyclopropane-1-carbonyl)-2,6-diazaspiro[3.4]octane-8-carboxylate (321 mg, 0.69 mmol) (synthesized in a similar fashion to ethyl 2-((S)-2,2-dimethylcyclopropane-1-carbonyl)-6-(2,4-dimethylthiazole-5-carbonyl)-2,6-diazaspiro[3.4]octane-8-carboxylate of I-66 using 2-(tert-butyl) 8-ethyl (S)-2,6-diazaspiro[3.4]octane-2,8-dicarboxylate) in anhydrous THF (5.0 mL) at −78° C. was added a solution of n-BuLi (1M in THF, 1.1 mL, 1.1 mmol). After stirring for 30 min, MeCN (45 mg, 1.1 mmol) was added and the reaction stirred at −70° C. for 4 h. The mixture was diluted with water (20 mL) and extracted with EtOAc (50 mL×2). The combined organic layers were washed with brine, dried over Na₂SO₄, filtered and concentrated. The residue was purified by column chromatography on silica gel (eluent: DCM:MeOH=20:1) to afford 3-(6-(1-benzyl-1H-pyrazole-4-carbonyl)-2-((S)-2,2-dimethylcyclopropane-1-carbonyl)-2,6-diazaspiro[3.4]octan-8-yl)-3-oxopropanenitrile (53 mg, 41%) as a white solid. LCMS m/z=460.3 [M+H]⁺.

### Step 2: (8-(5-aminoisoxazol-3-yl)-2-((S)-2,2-dimethylcyclopropane-1-carbonyl)-2,6-diazaspiro[3.4]octan-6-yl)(1-benzyl-1H-pyrazol-4-yl)methanone

To a solution of 3-(6-(1-benzyl-1H-pyrazole-4-carbonyl)-2-((S)-2,2-dimethylcyclopropane-1-carbonyl)-2,6-diazaspiro[3.4]octan-8-yl)-3-oxopropanenitrile (126 mg, 0.27 mmol) in DCM (2.0 mL) was added TEA (55 mg, 0.55 mmol) and NH₂OH·HCl (24 mg, 0.34 mmol). The reaction was heated at 60° C. for 6 h then was diluted with water (20 mL) and extracted with DCM (30 mL×2). The combined organic layers were washed with brine, dried over Na₂SO₄, filtered and concentrated. The residue was purified by column chromatography on silica gel (eluent: DCM:MeOH=15:1) to afford (8-(5-aminoisoxazol-3-yl)-2-((S)-2,2-dimethylcyclopropane-1-carbonyl)-2,6-diazaspiro[3.4]octan-6-yl)(1-benzyl-1H-pyrazol-4-yl)methanone (53 mg, 41%) as a white solid. LCMS m/z=475.3 [M+H]⁺.

### Step 3: N-(3-(6-(1-benzyl-1H-pyrazole-4-carbonyl)-2-((S)-2,2-dimethylcyclopropane-1-carbonyl)-2,6-diazaspiro[3.4]octan-8-yl)isoxazol-5-yl)cyclopropanecarboxamide

To a solution of (8-(5-aminoisoxazol-3-yl)-2-((S)-2,2-dimethylcyclopropane-1-carbonyl)-2,6-diazaspiro[3.4]octan-6-yl)(1-benzyl-1H-pyrazol-4-yl)methanone (55 mg, 0.12 mmol) and pyridine (14 mg, 0.17 mmol) in DCM (1.0 mL) was added cyclopropanecarbonyl chloride (16 mg, 0.15 mmol). The mixture was stirred at room temperature for 4 h then diluted with water (20 mL) and extracted with DCM (30 mL×3). The combined organic layers were washed with brine, dried over Na₂SO₄, filtered and concentrated. The residue was purified by prep-HPLC to afford N-(3-(6-(1-benzyl-1H-pyrazole-4-carbonyl)-2-((S)-2,2-dimethylcyclopropane-1-carbonyl)-2,6-diazaspiro[3.4]octan-8-yl)isoxazol-5-yl)cyclopropanecarboxamide (27 mg, 42%) as a white solid. LCMS m/z=543.4 [M+H]⁺; ¹H NMR (400 MHz, Methanol-d4) δ 8.28-8.21 (m, 1H), 7.94 (d, J=11.2 Hz, 1H), 7.42-7.21 (m, 5H), 6.41-6.29 (m, 1H), 5.37 (d, J=5.0 Hz, 2H), 4.46-3.63 (m, 10H), 1.83-1.73 (m, 1H), 1.41-1.30 (m, 1H), 1.20-1.06 (m, 4H), 1.01-0.91 (m, 7H), 0.77-0.68 (m, 1H).

### Synthesis of methyl 2-(6-(1-benzyl-1H-pyrazole-4-carbonyl)-2-((S)-2,2-dimethylcyclopropane-1-carbonyl)-2,6-diazaspiro[3.4]octan-8-yl)oxazole-4-carboxylate (I-86)

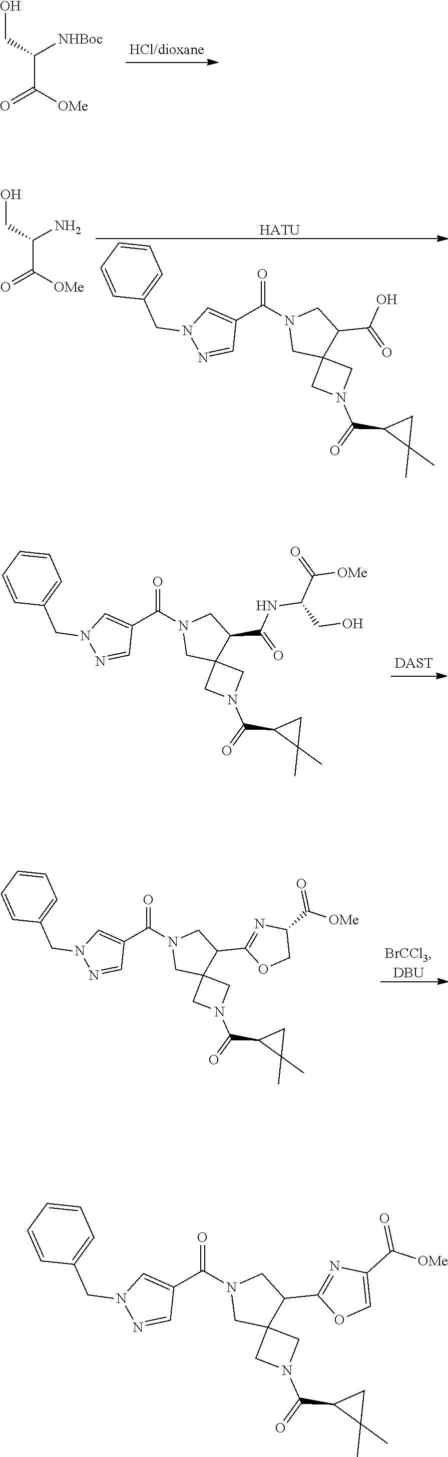

### Step 1: methyl L-serinate

To a solution of methyl (tert-butoxycarbonyl)-L-serinate (1.50 g, 6.84 mmol) in DCM (6 mL) was added HCl/dioxane (6 mL, 4.0 M in dioxane). The reaction mixture was stirred at room temperature for 1 h. The solvent was removed under vacuum to afford methyl L-serinate (1.12 g, 100%) which was used directly in the next step. LCMS m/z=120.1 [M+H]⁺.

### Step 2: methyl ((S)-6-(1-benzyl-1H-pyrazole-4-carbonyl)-2-((S)-2,2-dimethylcyclopropane-1-carbonyl)-2,6-diazaspiro[3.4]octane-8-carbonyl)-L-serinate

To a solution of 6-(1-benzyl-1H-pyrazole-4-carbonyl)-2-((S)-2,2-dimethylcyclopropane-1-carbonyl)-2,6-diazaspiro[3.4]octane-8-carboxylic acid (2.00 g, 4.58 mmol) in DCM (20 mL) was added HATU (1.74 g, 4.58 mmol) and DIPEA (2.37 g, 18.33 mmol). The reaction mixture was stirred at room temperature for 30 min. Methyl L-serinate (856 mg, 5.50 mmol) was then added and the mixture stirred at room temperature for another 2 h. The reaction was quenched with water (50 mL) and extracted with DCM (100 mL×2). The combined organic layers were washed with brine, dried over Na₂SO₄, filtered and concentrated. The residue was purified by RP-column to afford methyl ((S)-6-(1-benzyl-1H-pyrazole-4-carbonyl)-2-((S)-2,2-dimethylcyclopropane-1-carbonyl)-2,6-diazaspiro[3.4]octane-8-carbonyl)-L-serinate (1.56 g, 63%) as a yellowish solid. LCMS m/z=537.8 [M+H]⁺; ¹H NMR (400 MHz, DMSO-d₆) δ 8.74-8.57 (m, 1H), 8.40-8.31 (m, 1H), 7.88-7.77 (m, 1H), 7.39-7.21 (m, 5H), 5.35 (s, 2H), 5.15-5.05 (m, 1H), 4.41-4.32 (m, 1H), 4.19-3.86 (m, 4H), 3.79-3.56 (m, 8H), 3.49-3.32 (m, 2H), 1.41-1.31 (m, 1H), 1.14-1.03 (m, 6H), 0.91-0.82 (m, 1H), 0.74-0.63 (m, 1H).

### Step 3: methyl (4S)-2-(6-(1-benzyl-1H-pyrazole-4-carbonyl)-2-((S)-2,2-dimethylcyclopropane-1-carbonyl)-2,6-diazaspiro[3.4]octan-8-yl)-4,5-dihydrooxazole-4-carboxylate

To a solution of methyl ((S)-6-(1-benzyl-1H-pyrazole-4-carbonyl)-2-((S)-2,2-dimethylcyclopropane-1-carbonyl)-2,6-diazaspiro[3.4]octane-8-carbonyl)-L-serinate (200 mg, 0.37 mmol) in anhydrous DCM (2 mL) at −78° C. under a N₂ atmosphere was added dethylaminosulfur trifluoride (90 mg, 0.55 mmol). The reaction mixture was stirred at room temperature for 2 h then diluted with EtOAc (50 mL) and washed with brine (20 mL×2). The organic combined layers were dried over Na₂SO₄, filtered and concentrated to afford methyl (4S)-2-(6-(1-benzyl-1H-pyrazole-4-carbonyl)-2-((S)-2,2-dimethylcyclopropane-1-carbonyl)-2,6-diazaspiro[3.4]octan-8-yl)-4,5-dihydrooxazole-4-carboxylate (280 mg, crude) as a white solid. LCMS m/z=520.2 [M+H]⁺.

### Step 4: methyl 2-(6-(1-benzyl-1H-pyrazole-4-carbonyl)-2-((S)-2,2-dimethylcyclopropane-1-carbonyl)-2,6-diazaspiro[3.4]octan-8-yl)oxazole-4-carboxylate

To a solution of methyl (4S)-2-(6-(1-benzyl-1H-pyrazole-4-carbonyl)-2-((S)-2,2-dimethylcyclopropane-1-carbonyl)-2,6-diazaspiro[3.4]octan-8-yl)-4,5-dihydrooxazole-4-carboxylate (80 mg, 0.15 mmol) in anhydrous DCM (2 mL) at 0° C. was added 1,8-diazabicyclo[5.4.0]undec-7-ene (47 mg, 0.31 mmol) and bromotrichloromethane (73 mg, 0.37 mmol). The reaction mixture was stirred at room temperature overnight then concentrated. The residue obtained was purified by RP-column to afford methyl 2-(6-(1-benzyl-1H-pyrazole-4-carbonyl)-2-((S)-2,2-dimethylcyclopropane-1-carbonyl)-2,6-diazaspiro[3.4]octan-8-yl)oxazole-4-carboxylate (21 mg, 26%) as a white solid. LCMS m/z=518.2 [M+H]⁺; ¹H NMR (400 MHz, DMSO-d₆) δ 8.93-8.86 (m, 1H), 8.43-8.36 (m, 1H), 7.90-7.81 (m, 1H), 7.37-7.25 (m, 5H), 5.37 (s, 2H), 4.28-3.83 (m, 8H), 3.82-3.79 (m, 3H), 3.74 (s, 1H), 1.37-1.24 (m, 2H), 1.14-1.07 (m, 2H), 1.03 (d, J=8.2 Hz, 2H), 0.98-0.89 (m, 1H), 0.84 (s, 1H), 0.71-0.60 (m, 1H).

### Synthesis of N-benzyl-2-(6-(1-benzyl-1H-pyrazole-4-carbonyl)-2-((S)-2,2-dimethylcyclopropane-1-carbonyl)-2,6-diazaspiro[3.4]octan-8-yl)oxazole-4-carboxamide (I-91)

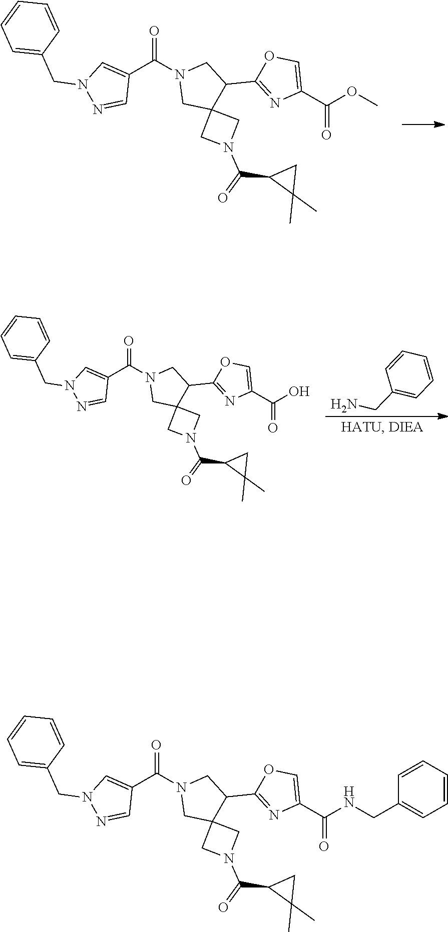 [SMILES: COC(=O)c1coc(C2CN(C(=O)c3cnn(Cc4ccccc4)c3)CC23CN(C(=O)[C@H]2CC2(C)C)C3)n1 ; CC1(C)C[C@@H]1C(=O)N1CC2(CN(C(=O)c3cnn(Cc4ccccc4)c3)CC2c2nc(C(=O)O)co2)C1 ; NCc1ccccc1 ; CC1(C)C[C@@H]1C(=O)N1CC2(CN(C(=O)c3cnn(Cc4ccccc4)c3)CC2c2nc(C(=O)NCc3ccccc3)co2)C1]

### Step 1: 2-(6-(1-benzyl-1H-pyrazole-4-carbonyl)-2-((S)-2,2-dimethylcyclopropane-1-carbonyl)-2,6-diazaspiro[3.4]octan-8-yl)oxazole-4-carboxylic acid

To a solution of methyl 2-(6-(1-benzyl-1H-pyrazole-4-carbonyl)-2-((S)-2,2-dimethylcyclopropane-1-carbonyl)-2,6-diazaspiro[3.4]octan-8-yl)oxazole-4-carboxylate (50 mg, 0.096 mmol) in a mixture of THF, MeOH and H₂O (1 mL/0.25 mL/0.25 mL) was added LiOH (7 mg, 0.28 mmol). The reaction was stirred at room temperature for 2 h. then diluted with water (10 mL) and extracted with EtOAc (10 mL). The aqueous layer was acidified to pH˜2 with 1M HCl and extracted with EtOAc (10 mL×2). The combined organic layers were washed with brine, dried over Na₂SO₄, filtered and concentrated to afford 2-(6-(1-benzyl-1H-pyrazole-4-carbonyl)-2-((S)-2,2-dimethylcyclopropane-1-carbonyl)-2,6-diazaspiro[3.4]octan-8-yl)oxazole-4-carboxylic acid (45 mg, 93%) as a white solid which was used directly in the next step. ¹H NMR (400 MHz, DMSO-d₆) δ 8.76-8.65 (m, 1H), 8.44-8.36 (m, 1H), 7.90-7.82 (m, 1H), 7.36-7.25 (m, 5H), 5.37 (s, 2H), 4.14-3.73 (m, 9H), 1.78-1.62 (m, 1H), 1.17-0.95 (m, 6H), 0.88-0.80 (m, 2H).

### Step 2: N-benzyl-2-(6-(1-benzyl-1H-pyrazole-4-carbonyl)-2-((S)-2,2-dimethylcyclopropane-1-carbonyl)-2,6-diazaspiro[3.4]octan-8-yl)oxazole-4-carboxamide

To a solution of 2-(6-(1-benzyl-1H-pyrazole-4-carbonyl)-2-((S)-2,2-dimethylcyclopropane-1-carbonyl)-2,6-diazaspiro[3.4]octan-8-yl)oxazole-4-carboxylic acid (50 mg, 0.10 mmol) in DCM (2 mL) was added HATU (41 mg, 0.11 mmol) and DIPEA (51 mg, 0.40 mmol). The mixture was stirred at room temperature for 30 min then benzylamine (11 mg, 0.10 mmol) was added and the reaction stirred for another 2 h. The mixture was diluted with water (10 mL) and extracted with DCM (30 mL×3). The combined organic layers were washed with brine, dried over Na₂SO₄, filtered and concentrated. The residue obtained was purified by prep-HPLC to afford N-benzyl-2-(6-(1-benzyl-1H-pyrazole-4-carbonyl)-2-((S)-2,2-dimethylcyclopropane-1-carbonyl)-2,6-diazaspiro[3.4]octan-8-yl)oxazole-4-carboxamide (19.8 mg, 34% yield) as a white solid. LCMS m/z=593.2 ¹H NMR (400 MHz, DMSO-d₆) δ 8.81-8.59 (m, 2H), 8.41-8.35 (m, 1H), 7.89-7.83 (m, 1H), 7.38-7.20 (m, 10H), 5.36 (s, 2H), 4.45-4.35 (m, 2H), 4.31-4.15 (m, 2H), 4.14-3.96 (m, 3H), 3.96-3.85 (m, 3H), 3.83-3.73 (m, 1H), 1.38-1.25 (m, 1H), 1.13-1.00 (m, 4H), 0.98-0.92 (m, 1H), 0.85-0.78 (m, 2H), 0.69-0.57 (m, 1H).

Table 9: The compounds listed in Table 9 were synthesized from 2-(6-(1-benzyl-1H-pyrazole-4-carbonyl)-2-((S)-2,2-dimethylcyclopropane-1-carbonyl)-2,6-diazaspiro[3.4]octan-8-yl)oxazole-4-carboxylic acid according to the procedures outlined for I-91 using the appropriate commercially available reagents and/or intermediates described elsewhere.

TABLE 9

| Example | | | |
|---|---|---|---|
| Number | Compound | ¹HNMR | LCMS |
| I-88 | 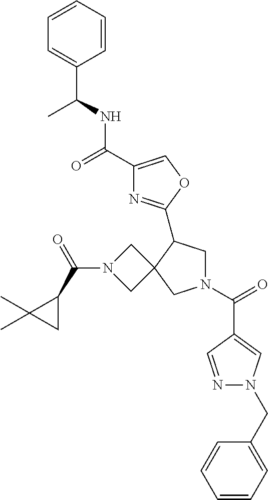 [SMILES: C[C@H](NC(=O)c1coc(C2CN(C(=O)c3cnn(Cc4ccccc4)c3)CC23CN(C(=O)[C@H]2CC2(C)C)C3)n1)c1ccccc1] | ¹H NMR (400 MHz, DMSO-d6) δ 8.67-8.60 (m, 1H), 8.53-8.38 (m, 2H), 7.92-7.84 (m, 1H), 7.42-7.23 (m, 10H), 5.39 (s, 2H), 5.20-5.11 (m, 1H), 4.31-3.87 (m, 8H), 3.83-3.76 (m, 1H), 1.54-1.47 (m, 3H), 1.42- 1.33 (m, 1H), 1.17- 1.03 (m, 4H), 0.97-0.95 (m, 1H), 0.87-0.79 (m, 2H), 0.72-0.58 (m, 1H). | m/z = 607.3 [M + H]⁺ |
| I-11 | 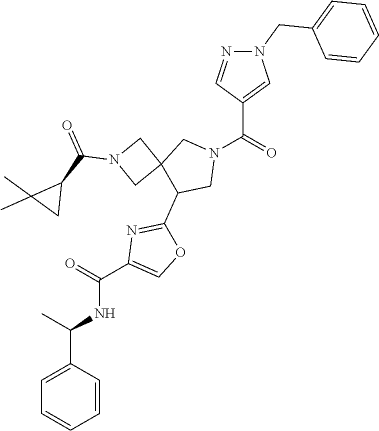 [SMILES: C[C@@H](NC(=O)c1coc(C2CN(C(=O)c3cnn(Cc4ccccc4)c3)CC23CN(C(=O)[C@H]2CC2(C)C)C3)n1)c1ccccc1] | ¹H NMR (400 MHz, DMSO-d₆) δ 8.65-8.57 (m, 1H), 8.54-8.42 (m, 1H), 8.41-8.33 (m, 1H), 7.90-7.82 (m, 1H), 7.42- 7.15 (m, 10H), 5.36 (s, 2H), 5.19-5.09 (m, 1H), 4.31-3.75 (m, 9H), 1.51-1.44 (m, 3H), 1.39- 1.32 (m, 1H), 1.28-1.22 (m, 1H), 1.13-1.10 (m, 1H), 1.05-1.00 (m, 2H), 0.96-0.89 (m, 1H), 0.85- 0.77 (m, 2H), 0.70- 0.55 (m, 1H). | m/z = 607.2 [M + H]⁺ |
| I-89 | 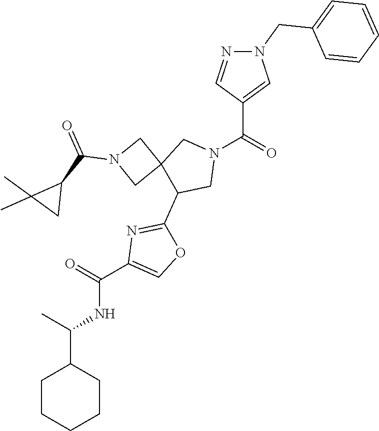 [SMILES: C[C@H](NC(=O)c1coc(C2CN(C(=O)c3cnn(Cc4ccccc4)c3)CC23CN(C(=O)[C@H]2CC2(C)C)C3)n1)C1CCCCC1] | ¹H NMR (400 MHz, DMSO-d₆) δ 8.61-8.54 (m, 1H), 8.39 (d, J = 11.2Hz, 1H), 7.86 (m, 1H), 7.70 (m, 1H), 7.37- 7.23 (m, 5H), 5.37 (s, 2H), 4.31-3.98 (m, 5H), 3.91 (d, J = 6.2Hz, 2H), 3.81-3.59 (m, 3H), 1.69 (s, 5H), 1.60 (s, 1H), 1.46- 1.32 (m, 2H), 1.25 (d, J = 6.2 Hz, 3H), 1.12- 1.08 (m, 6H), 1.01-0.81 (m, 6H). | m/z = 613.4 [M + H]⁺ |
| I-90 | 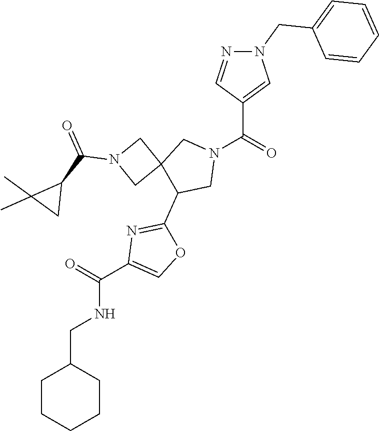 [SMILES: CC1(C)C[C@@H]1C(=O)N1CC2(CN(C(=O)c3cnn(Cc4ccccc4)c3)CC2c2nc(C(=O)NCC3CCCCC3)co2)C1] | ¹H NMR (400 MHz, DMSO-d₆) δ 8.61-8.54 (m, 1H), 8.46-8.35 (m, 1H), 8.17-8.05 (m, 1H), 7.90-7.82 (m, 1H), 7.39- 7.22 (m, 5H), 5.36 (s, 2H), 4.39-4.20 (m, 1H), 4.20-4.05 (m, 3H), 4.04- 3.87 (m, 4H), 3.82- 3.73 (m, 1H), 3.10-3.00 (m, 2H), 1.69-1.62 (m, 4H), 1.50 (s, 1H), 1.31 (m, 2H), 1.16-0.99 (m, 7H), 0.96-0.77 (m, 5H), 0.71-0.58 (m, 1H). | m/z = 599.2 [M + H]⁺ |

### Synthesis of ((R)-8-(5-(3,4-dichlorobenzyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone (I-19)

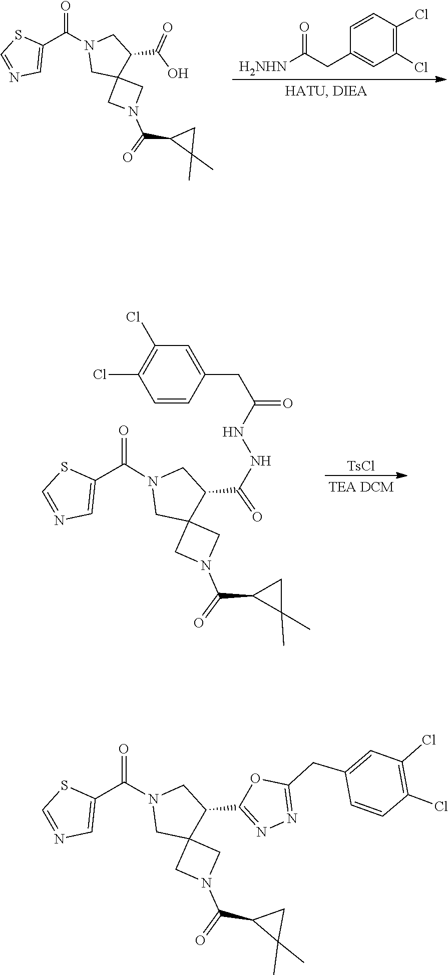

### Step 1: (R)—N′-(2-(3,4-dichlorophenyl)acetyl)-2-((S)-2,2-dimethylcyclopropane-1-carbonyl)-6-(thiazole-5-carbonyl)-2,6-diazaspiro[3.4]octane-8-carbohydrazide

To a solution of (R)-2-((S)-2,2-dimethylcyclopropane-1-carbonyl)-6-(thiazole-5-carbonyl)-2,6-diazaspiro[3.4]octane-8-carboxylic acid (259 mg, 0.71 mmol) in DCM (4 mL) was added HATU (270 mg, 0.71 mmol) and DIPEA (275 mg, 2.13 mmol). The mixture was stirred at room temperature for 30 min then 2-(3,4-dichlorophenyl)acetohydrazide (155 mg, 0.71 mmol) was added. The reaction mixture was stirred at room temperature for 4 h then diluted with water (30 mL) and extracted with DCM (50 mL×3). The combined organic layers were washed with brine, dried over Na₂SO₄, filtered and concentrated. The residue was purified by prep-TLC (eluent: DCM:MeOH=10:1) to afford (R)—N′-(2-(3,4-dichlorophenyl)acetyl)-2-((S)-2,2-dimethylcyclopropane-1-carbonyl)-6-(thiazole-5-carbonyl)-2,6-diazaspiro[3.4]octane-8-carbohydrazide (110 mg, 28%) as a yellow solid. LCMS m/z=564.6 [M+H]⁺.

### Step 7: ((R)-8-(5-(3,4-dichlorobenzyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone

To a solution of (R)—N′-(2-(3,4-dichlorophenyl)acetyl)-2-((S)-2,2-dimethylcyclopropane-1-carbonyl)-6-(thiazole-5-carbonyl)-2,6-diazaspiro[3.4]octane-8-carbohydrazide (100 mg, 0.18 mmol) in DCM (2 mL) was added TEA (90 mg, 0.89 mmol) and TsCl (102 mg, 0.53 mmol). The reaction was stirred at room temperature for 2 h then diluted with water (30 mL) and extracted with DCM (50 mL×3). The combined organic layers were washed with brine, dried over Na₂SO₄, filtered and concentrated. The residue obtained was purified by prep-TLC (eluent: DCM:MeOH=1) to afford ((R)-8-(5-(3,4-dichlorobenzyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone (61.3 mg, 63%) as a white solid. LCMS m/z=544.1 [M−H]⁻; ¹H NMR (400 MHz, DMSO-d₆) δ 9.27 (s, 1H), 8.38 (d, J=20.4 Hz, 1H), 7.63 (d, J=23.4 Hz, 2H), 7.32 (s, 1H), 4.31 (t, J=6.2 Hz, 3H), 4.27-4.14 (m, 2H), 4.12 (d, J=8.5 Hz, 1H), 4.06 (d, J=9.6 Hz, 1H), 3.95-3.85 (m, 3H), 3.82 (d, J=11.8 Hz, 1H), 1.22 (d, J=12.4 Hz, 1H), 1.09 (d, J=10.9 Hz, 3H), 1.03 (d, J=8.8 Hz, 2H), 0.98 (d, J=4.6 Hz, 1H), 0.83 (s, 1H), 0.64 (s, 1H).

### Synthesis of ((S)-8-(5-(3,4-dichlorobenzyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone (I-19)

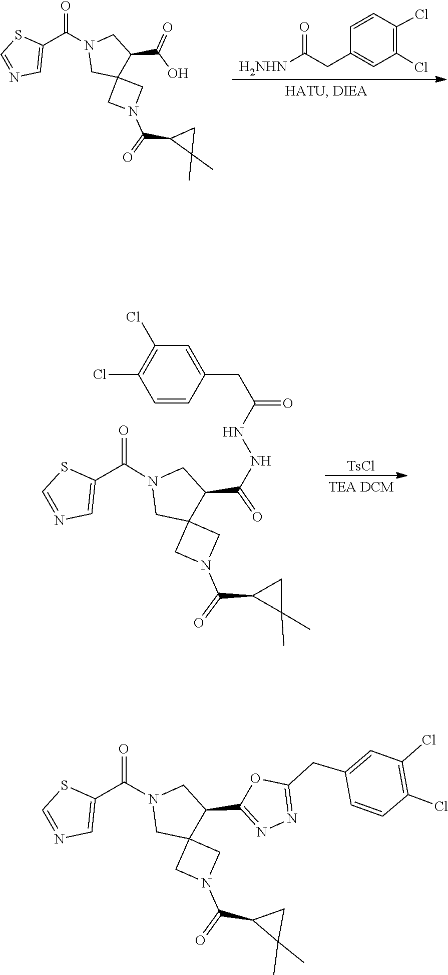

### Step 1: (S)—N′-(2-(3,4-dichlorophenyl)acetyl)-2-((S)-2,2-dimethylcyclopropane-1-carbonyl)-6-(thiazole-5-carbonyl)-2,6-diazaspiro[3.4]octane-8-carbohydrazide

To a solution of (S)-2-((S)-2,2-dimethylcyclopropane-1-carbonyl)-6-(thiazole-5-carbonyl)-2,6-diazaspiro[3.4]octane-8-carboxylic acid (100 mg, 0.27 mmol) in DCM (2 mL) was added HATU (103 mg, 0.27 mmol) and DIPEA (140 mg, 1.08 mmol). The mixture was stirred at room temperature for 30 min then 2-(3,4-dichlorophenyl)acetohydrazide (59 mg, 0.27 mmol) was added and the reaction mixture stirred another 2 h. The reaction was diluted with water (30 mL) and extracted with DCM (50 mL×2). The combined organic layers were washed with brine, dried over Na₂SO₄, filtered and concentrated. The residue obtained was purified by prep-TLC (eluent: DCM:MeOH=10:1, v/v) to afford (S)—N′-(2-(3,4-dichlorophenyl)acetyl)-2-((S)-2,2-dimethylcyclopropane-1-carbonyl)-6-(thiazole-5-carbonyl)-2,6-diazaspiro[3.4]octane-8-carbohydrazide (23 mg, 15%) as a yellow solid. LCMS m/z=564.0 [M+H]⁺.

### Step 4: ((S)-8-(5-(3,4-dichlorobenzyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone

To a solution of (S)—N′-(2-(3,4-dichlorophenyl)acetyl)-2-((S)-2,2-dimethylcyclopropane-1-carbonyl)-6-(thiazole-5-carbonyl)-2,6-diazaspiro[3.4]octane-8-carbohydrazide (80 mg, 0.18 mmol) in DCM (2 mL) was added TEA (70 mg, 0.70 mmol) and TsCl (81 mg, 0.42 mmol). The mixture was stirred at room temperature for 2 h then diluted with water (30 mL) and extracted with DCM (50 mL×3). The combined organic layers were washed with brine, dried over Na₂SO₄, filtered and concentrated. The residue was purified by prep-TLC (eluent: DCM:MeOH=15:1) to afford ((S)-8-(5-(3,4-dichlorobenzyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone (50 mg, 63%) as a white solid. LCMS m/z=546.2 [M+H]⁺; ¹H NMR (400 MHz, DMSO-d₆) δ 9.29-9.25 (m, 1H), 8.39 (d, J=17.8 Hz, 1H), 7.67-7.57 (m, 2H), 7.32 (t, J=8.2 Hz, 1H), 4.35-4.18 (m, 5H), 4.10-3.87 (m, 5H), 3.84-3.73 (m, 1H), 1.31-1.25 (m, 1H), 1.14-1.08 (m, 2H), 1.07-1.01 (m, 3H), 0.90 (s, 1H), 0.84 (s, 1H), 0.70-0.63 (m, 1H).

### Synthesis of (2-((S)-2,2-dimethylcyclopropane-1-carbonyl)-8-(5-((6-(tetrahydro-2H-pyran-4-yl)pyridin-2-yl)methyl)-1,3,4-oxadiazol-2-yl)-2,6-diazaspiro[3.4]octan-6-yl)(2,4-dimethylthiazol-5-yl)methanone (I-66)

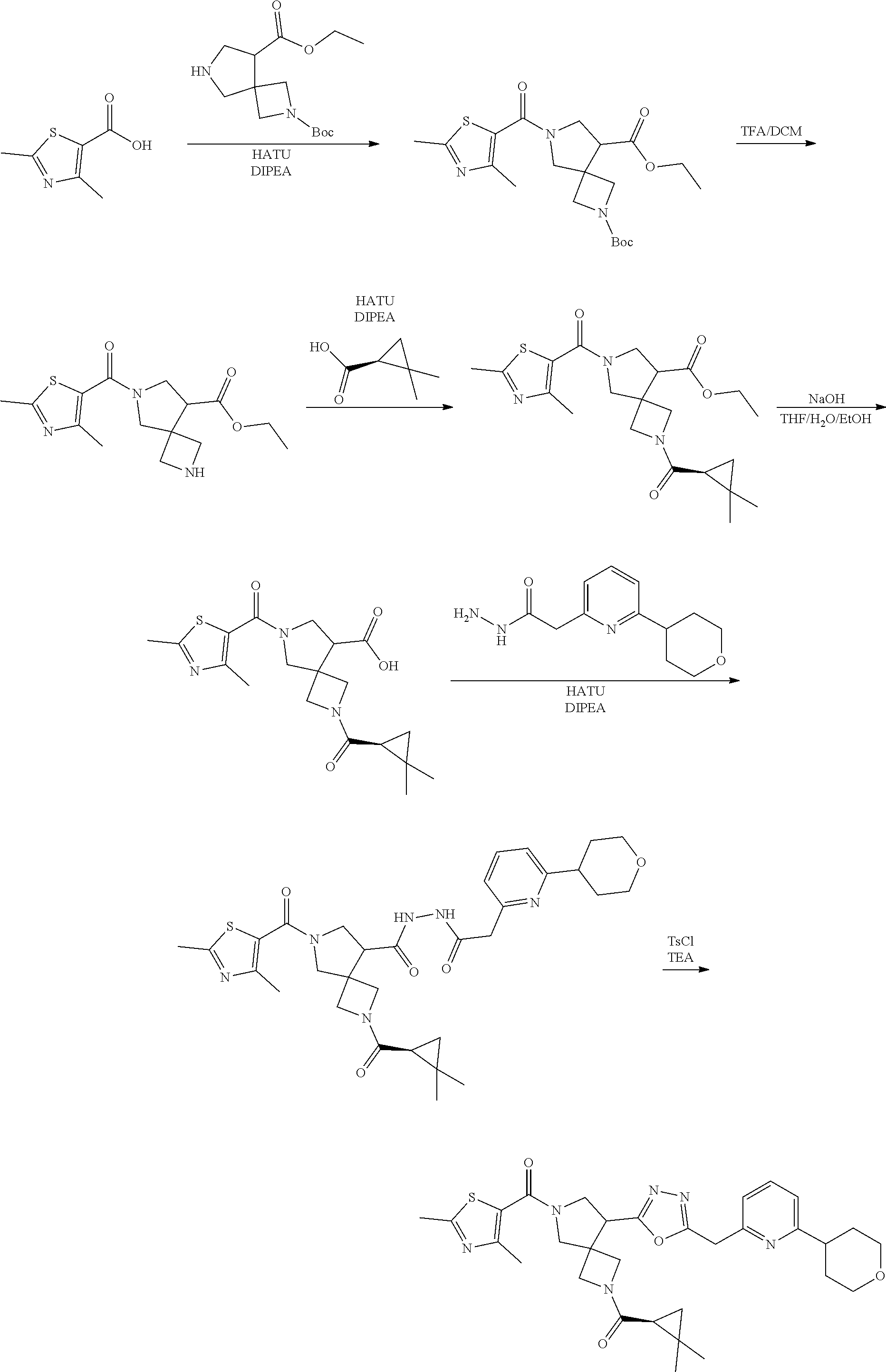

### Step 1: 2-(tert-butyl) 8-ethyl 6-(2,4-dimethylthiazole-5-carbonyl)-2,6-diazaspiro[3.4]octane-2,8-dicarboxylate

To a solution of 2,4-dimethylthiazole-5-carboxylic acid (5 g, 31.8 mmol) in DCM (100 mL) was added HATU (13 g, 35.0 mmol) and and DIPEA (16 g, 127.2 mmol). The mixture was stirred at room temperature for 30 min then 2-(tert-butyl) 8-ethyl 2,6-diazaspiro[3.4]octane-2,8-dicarboxylate (10.8 g, 38.2 mmol) was added and the reaction was stirred for another 2 h. The mixture was diluted with water (500 mL) and extracted with DCM (500 mL×3). The combined organic layers were washed with brine, dried over Na₂SO₄, filtered and concentrated. The residue obtained was purified by column chromatography on silica gel (eluent: DCM:MeOH=80:1) to afford 2-(tert-butyl) 8-ethyl 6-(2,4-dimethylthiazole-5-carbonyl)-2,6-diazaspiro[3.4]octane-2,8-dicarboxylate (11.2 g, 86%) as a yellow solid. LCMS m/z=424.3 [M+H]⁺; ¹H NMR (400 MHz, DMSO-d₆) δ 3.80-3.61 (m, 10H), 2.69 (s, 3H), 2.62 (s, 3H), 2.27-2.20 (m, 1H), 1.37-1.36 (m, 9H), 1.20-1.17 (in, 3H).

### Step 2: ethyl 6-(2,4-dimethylthiazole-5-carbonyl)-2,6-diazaspiro[3.4]octane-8-carboxylate

To a solution of 2-(tert-butyl) 8-ethyl 6-(2,4-dimethylthiazole-5-carbonyl)-2,6-diazaspiro[3.4]octane-2,8-dicarboxylate (205 mg, 0.5 mmol) in DCM (2 mL) was added TF A (0.5 mL) and the reaction stirred at room temperature for 1 h. The solvent was removed under vacuum to afford ethyl 6-(2,4-dimethylthiazole-5-carbonyl)-2,6-diazaspiro[3.4]octane-8-carboxylate (156 mg, 100%) which was used directly in the next step. LCMS m/z=324.1 [M+H]⁺.

### Step 3: ethyl 2-((S)-2,2-dimethylcyclopropane-1-carbonyl)-6-(2,4-dimethylthiazole-5-carbonyl)-2,6-diazaspiro[3.4]octane-8-carboxylate

To a solution of ethyl 6-(2,4-dimethylthiazole-5-carbonyl)-2,6-diazaspiro[3.4]octane-8-carboxylate (156 mg, 0.5 mmol) in DCM (2 mL) was added HATU (201 mg, 0.5 mmol) and DIPEA (249 mg, 1.9 mmol). The mixture was stirred at room temperature for 30 min then (S)-2,2-dimethylcyclopropane-1-carboxylic acid (60 mg, 0.5 mmol) was added and the reaction stirred for another 2 h. The mixture was diluted with water (20 mL) and extracted with DCM (50 mL×3). The combined organic layers were washed with brine, dried over Na₂SO₄, filtered and concentrated. The residue obtained was purified by prep-TLC (eluent: DCM:MeOH=20:1) to afford ethyl 2-((S)-2,2-dimethylcyclopropane-1-carbonyl)-6-(2,4-dimethylthiazole-5-carbonyl)-2,6-diazaspiro[3.4]octane-8-carboxylate (130 mg, 64%) as a white solid. LCMS m/z=420.1 [M+H]⁺; ¹H NMR (400 MHz, DMSO-d₆) δ 4.33-3.55 (m, 10H), 2.69 (s, 1H), 2.63 (s, 3H), 2.35 (s, 3H), 1.40-1.29 (m, 1H), 1.21-1.13 (m, 3H), 1.13-0.99 (m, 5H), 0.85 (d, J=5.3 Hz, 1H), 0.67 (d, J=7.0 Hz, 1H).

### Step 4: 2-((S)-2,2-dimethylcyclopropane-1-carbonyl)-6-(2,4-dimethylthiazole-5-carbonyl)-2,6-diazaspiro[3.4]octane-8-carboxylic acid

To a solution of ethyl 2-((S)-2,2-dimethylcyclopropane-1-carbonyl)-6-(2,4-dimethylthiazole-5-carbonyl)-2,6-diazaspiro[3.4]octane-8-carboxylate (130 mg, 0.31 mmol) in a mixture of THF, water and EtOH (0.8 mL/0.2 mL/0.2 mL) at 40° C. was added NaOH (25 mg, 0.42 mmol). The reaction was heated at 40° C. for 2 h then diluted with water (10 mL) and extracted with EtOAc (20 mL×2). The aqueous layer was collected and acidified to pH˜3 with 1M HCl then extracted with EtOAc (30 mL×3). The combined organic layers were washed with brine, dried over Na₂SO₄, filtered and concentrated to afford 2-((S)-2,2-dimethylcyclopropane-1-carbonyl)-6-(2,4-dimethylthiazole-5-carbonyl)-2,6-diazaspiro[3.4]octane-8-carboxylic acid (78 mg, 64%) as a white solid which was used directly in the next step. LCMS m/z=392.1 [M+H]⁺.

### Step 5: 2-((S)-2,2-dimethylcyclopropane-1-carbonyl)-6-(2,4-dimethylthiazole-5-carbonyl)-N′-(2-(6-(tetrahydro-2H-pyran-4-yl)pyridin-2-yl)acetyl)-2,6-diazaspiro[3.4]octane-8-carbohydrazide

To a solution of 2-((S)-2,2-dimethylcyclopropane-1-carbonyl)-6-(2,4-dimethylthiazole-5-carbonyl)-2,6-diazaspiro[3.4]octane-8-carboxylic acid (115 mg, 0.3 mmol) in DCM (2 mL) was added HATU (145 mg, 0.38 mmol) and DWEA (113 mg, 0.9 mmol). The mixture was stirred at room temperature for 30 min then 2-(6-(tetrahydro-2H-pyran-4-yl)pyridin-2-yl)acetohydrazide (69 mg, 0.29 mmol) was added and the reaction stirred for another 2 h. The mixture was diluted with water (20 mL) and extracted with DCM (30 mL×3). The combined organic layers were washed with brine, dried over Na₂SO₄, filtered and concentrated. The residue obtained was purified by prep-TLC (eluent: DCM:MeOH=15:1) to afford 2-((S)-2,2-dimethylcyclopropane-1-carbonyl)-6-(2,4-dimethylthiazole-5-carbonyl)-N′-(2-(6-(tetrahydro-2H-pyran-4-yl)pyridin-2-yl)acetyl)-2,6-diazaspiro[3.4]octane-8-carbohydrazide (77 mg, 41%) as a white solid. LCMS m/z=609.2 [M+H]⁺; ¹H NMR (400 MHz, DMSO-d₆) δ 10.29-10.14 (m, 2H), 7.71-7.63 (m, 1H), 7.24-7.13 (m, 2H), 4.03-3.61 (m, 12H), 3.47-3.42 (m, 2H), 3.24-3.04 (m, 2H), 2.65-2.56 (m, 3H), 2.35 (s, 3H), 1.77-1.69 (m, 4H), 1.26-1.24 (m, 3H), 1.10-1.06 (m, 2H), 1.05-1.00 (m, 2H), 0.90-0.79 (m, 1H), 0.70-0.61 (m, 1H).

### Step 6 (2-((S)-2,2-dimethylcyclopropane-1-carbonyl)-8-(5-((6-(tetrahydro-2H-pyran-4-yl)pyridin-2-yl)methyl)-1,3,4-oxadiazol-2-yl)-2,6-diazaspiro[3.4]octan-6-yl)(2,4-dimethylthiazol-5-yl)methanone

To a solution of 2-((S)-2,2-dimethylcyclopropane-1-carbonyl)-6-(2,4-dimethylthiazole-5-carbonyl)-N′-(2-(6-(tetrahydro-2H-pyran-4-yl)pyridin-2-yl)acetyl)-2,6-diazaspiro[3.4]octane-8-carbohydrazide (47 mg, 0.08 mmol) in DCM (1 mL) was added TEA (587 mg, 5.82 mmol) and TsCl (29 mg, 0.15 mmol). The reaction mixture was stirred at room temperature for 2 hours then concentrated to afford the crude. The residue was purified by prep-HPLC to give (2-((S)-2,2-dimethylcyclopropane-1-carbonyl)-8-(5-((6-(tetrahydro-2H-pyran-4-yl)pyridin-2-yl)methyl)-1,3,4-oxadiazol-2-yl)-2,6-diazaspiro[3.4]octan-6-yl)(2,4-dimethylthiazol-5-yl)methanone (3.1 mg, 7%) as a white solid. LCMS m/z=591.4 [M+H]⁺; ¹H NMR (400 MHz, Chloroform-d) δ 7.84-7.71 (m, 1H), 7.26-7.13 (m, 2H), 4.59-4.50 (m, 1H), 4.36-4.27 (m, 1H), 4.08-3.97 (m, 5H), 3.88-3.73 (m, 2H), 3.60-3.50 (m, 2H), 2.69 (s, 3H), 2.52-2.44 (m, 3H), 1.95-1.77 (m, 5H), 1.28-1.23 (m, 2H), 1.20-1.07 (m, 9H), 0.91-0.82 (m, 1H), 0.78-0.70 (m, 1H).

Table 10. The compounds listed in Table 10 were synthesized from 2-((S)-2,2-dimethylcyclopropane-1-carbonyl)-6-(2,4-dimethylthiazole-5-carbonyl)-2,6-diazaspiro[3.4]octane-8-carboxylic acid according to the procedures outlined for I-66 using the appropriate commercially available reagents and/or intermediates described elsewhere.

TABLE 10

| Example | | | |
|---|---|---|---|
| Number | Compound | ¹HNMR | LCMS |
| I-104 | 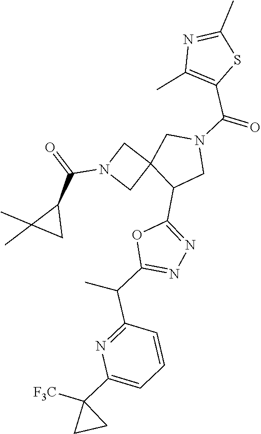 [SMILES: Cc1nc(C)c(C(=O)N2CC(c3nnc(C(C)c4cccc(C5(C(F)(F)F)CC5)n4)o3)C3(C2)CN(C(=O)[C@H]2CC2(C)C)C3)s1] | ¹H NMR (400 MHz, DMSO-d₆) δ 7.86-7.75 (m, 1H), 7.48-7.41 (m, 1H), 7.37-7.25 (m, 1H), 4.69-4.54 (m, 1H), 4.25- 3.65 (m, 9H), 2.62 (s, 3H), 2.31 (s, 3H), 1.63 (d, J = 7.0 Hz, 3H), 1.38- 1.32 (m, 2H), 1.30- 1.22 (m, 3H), 1.12-0.92 (m, 6H), 0.87-0.80 (m, 1H), 0.71-0.61 (m, 1H). | m/z = 629.3 [M + H]⁺ |

### Synthesis of (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(2,4-dimethylthiazol-5-yl)methanone (I-103)

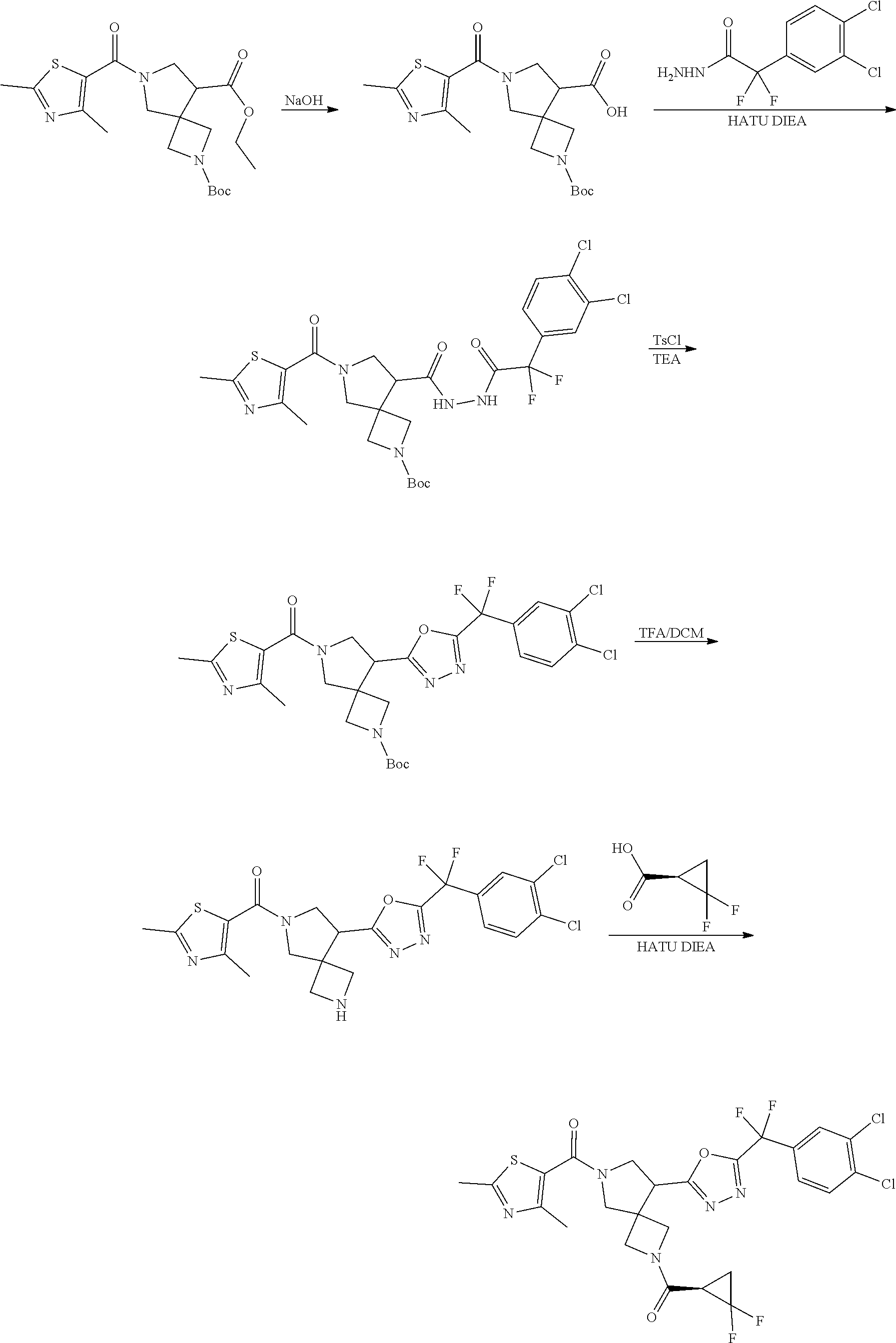

### Step 1: 2-(tert-butoxycarbonyl)-6-(2,4-dimethylthiazole-5-carbonyl)-2,6-diazaspiro[3.4]octane-8-carboxylic acid

To a solution of 2-(tert-butyl) 8-ethyl 6-(2,4-dimethylthiazole-5-carbonyl)-2,6-diazaspiro[3.4]octane-2,8-dicarboxylate (600 mg, 1.4 mmol) in a mixture of THF, water and EtOH (4.0 mL/1.0 mL/1.0 mL) was added NaOH (170 mg, 4.2 mmol). The reaction mixture was stirred at room temperature for 3 h, diluted with water (20 mL) and extracted with ether (40 mL). The aqueous layer was collected and acidified to pH˜2 with 1M HCl and extracted with EtOAc (50 mL×3). The combined organic layers was washed with brine, dried over Na₂SO₄, filtered and concentrated to afford 2-(tert-butoxycarbonyl)-6-(2,4-dimethylthiazole-5-carbonyl)-2,6-diazaspiro[3.4]octane-8-carboxylic acid (500 mg, 89%) as a white solid. LCMS m/z=396.1 [M+H]⁺; ¹H NMR (400 MHz, Chloroform-d) δ 4.07-3.63 (m, 9H), 2.69 (s, 3H), 2.48 (s, 3H), 1.43 (s, 9H).

### Step 2: tert-butyl 8-(2-(2-(3,4-dichlorophenyl)-2,2-difluoroacetyl)hydrazine-1-carbonyl)-6-(2,4-dimethylthiazole-5-carbonyl)-2,6-diazaspiro[3.4]octane-2-carboxylate

To a solution of 2-(tert-butoxycarbonyl)-6-(2,4-dimethylthiazole-5-carbonyl)-2,6-diazaspiro[3.4]octane-8-carboxylic acid (200 mg, 0.51 mmol) in DCM (10 mL) was added HATU (194 mg, 0.51 mmol). The mixture was stirred at room temperature for 30 min then 2-(3,4-dichlorophenyl)-2,2-difluoroacetohydrazide (130 mg, 0.51 mmol) and DIPEA (197 mg, 1.53 mmol) were added and the reaction stirred at room temperature for another 3 h. The mixture was diluted with water (20 mL) and extracted with DCM (50 mL×3). The combined organic layers were washed with brine, dried over Na₂SO₄, filtered and concentrated. The residue obtained was purified by prep-TLC (eluent: DCM:MeOH=20:1) to afford tert-butyl 8-(2-(2-(3,4-dichlorophenyl)-2,2-difluoroacetyl)hydrazine-1-carbonyl)-6-(2,4-dimethylthiazole-5-carbonyl)-2,6-diazaspiro[3.4]octane-2-carboxylate (280 mg, 70%) as a white solid. LCMS m/z=632.1 [M+H]⁺; 1H NMR (400 MHz, Chloroform-d) δ 7.74 (s, 1H), 7.54 (d, J=8.4 Hz, 1H), 7.47 (d, J=8.8 Hz, 1H), 4.14 (d, J=9.6 Hz, 1H), 3.96-3.61 (m, 7H), 3.25-3.17 (m, 1H), 2.67 (s, 3H), 2.47 (s, 3H), 1.42 (s, 9H).

### Step 3: tert-butyl 8-(5-((3,4-dichlorophenyl)difluoromethyl)-1,3,4-oxadiazol-2-yl)-6-(2,4-dimethylthiazole-5-carbonyl)-2,6-diazaspiro[3.4]octane-2-carboxylate

To a solution of tert-butyl 8-(2-(2-(3,4-dichlorophenyl)-2,2-difluoroacetyl)hydrazine-1-carbonyl)-6-(2,4-dimethylthiazole-5-carbonyl)-2,6-diazaspiro[3.4]octane-2-carboxylate (200 mg, 0.51 mmol) in DCM (10 mL) was added TEA (258 mg, 2.55 mmol) and TsCl (292 mg, 1.53 mmol). The reaction mixture was stirred at room temperature for 2 h. then diluted with water (30 mL), extracted with DCM (50 mL×3). The combined organic layers were washed with brine, dried over Na₂SO₄, filtered and concentrated. The residue obtained was purified by prep-TLC (eluent: DCM:MeOH=15:1) to afford tert-butyl 8-(5-((3,4-dichlorophenyl)difluoromethyl)-1,3,4-oxadiazol-2-yl)-6-(2,4-dimethylthiazole-5-carbonyl)-2,6-diazaspiro[3.4]octane-2-carboxylate (230 mg, 72%) as a white solid. LCMS m/z=614.1 [M+H]⁺; ¹H NMR (400 MHz, Chloroform-d) δ 7.77 (s, 1H), 7.61 (d, J=8.4 Hz, 1H), 7.49 (dd, J=8.4, 2.0 Hz, 1H), 4.11-3.78 (m, 10H), 2.69 (s, 3H), 2.49 (s, 3H), 1.41 (s, 10H).

### Step 4: (8-(5-((3,4-dichlorophenyl)difluoromethyl)-1,3,4-oxadiazol-2-yl)-2,6-diazaspiro[3.4]octan-6-yl)(2,4-dimethylthiazol-5-yl)methanone

To a solution of tert-butyl 8-(5-((3,4-dichlorophenyl)difluoromethyl)-1,3,4-oxadiazol-2-yl)-6-(2,4-dimethylthiazole-5-carbonyl)-2,6-diazaspiro[3.4]octane-2-carboxylate (50 mg, 0.08 mmol) in DCM (4 mL) was added TFA (1 mL) and the reaction stirred at room temperature for 1 h. The solvent was removed under vacuum to afford (8-(5-((3,4-dichlorophenyl)difluoromethyl)-1,3,4-oxadiazol-2-yl)-2,6-diazaspiro[3.4]octan-6-yl)(2,4-dimethylthiazol-5-yl)methanone (42 mg, 100%) which was used directly in the next step. LCMS m/z=514 [M+H]⁺.

### Step 5: (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(2,4-dimethylthiazol-5-yl)methanone

To a solution of (R)-2,2-difluorocyclopropane-1-carboxylic acid (16 mg, 0.08 mmol) in DCM (5 mL) was added HATU (49 mg, 0.13 mmol) and DIPEA (50 mg, 0.39 mmol). The reaction was stirred at room temperature for 30 min then (8-(5-((3,4-dichlorophenyl)difluoromethyl)-1,3,4-oxadiazol-2-yl)-2,6-diazaspiro[3.4]octan-6-yl)(2,4-dimethylthiazol-5-yl)methanone (67 mg, 0.13 mmol) was added and stirring continued for another 2 h. The mixture was diluted with water (10 mL) and extracted with DCM (50 mL). The combined organic layers were washed with brine, dried over Na₂SO₄, filtered and concentrated. The residue obtained was purified by prep-HPLC to afford (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(2,4-dimethylthiazol-5-yl)methanone I-103 (70 mg, 54%) as a white solid. LCMS m/z=618.2 [M+H]⁺; ¹H NMR (400 MHz, DMSO-d6) δ 8.01 (s, 1H), 7.89 (d, J=8.4 Hz, 1H), 7.72 (s, 1H), 4.43-3.96 (m, 4H), 3.96-3.76 (m, 5H), 2.68-2.55 (m, 4H), 2.34 (s, 3H), 1.91-1.77 (m, 2H).

### Synthesis of (2-((S)-2,2-dimethylcyclopropane-1-carbonyl)-8-(5-((3-fluoro-6-(tetrahydro-2H-pyran-4-yl)pyridin-2-yl)methyl)-1,3,4-oxadiazol-2-yl)-2,6-diazaspiro[3.4]octan-6-yl)(thiazol-5-yl)methanone (I-50)

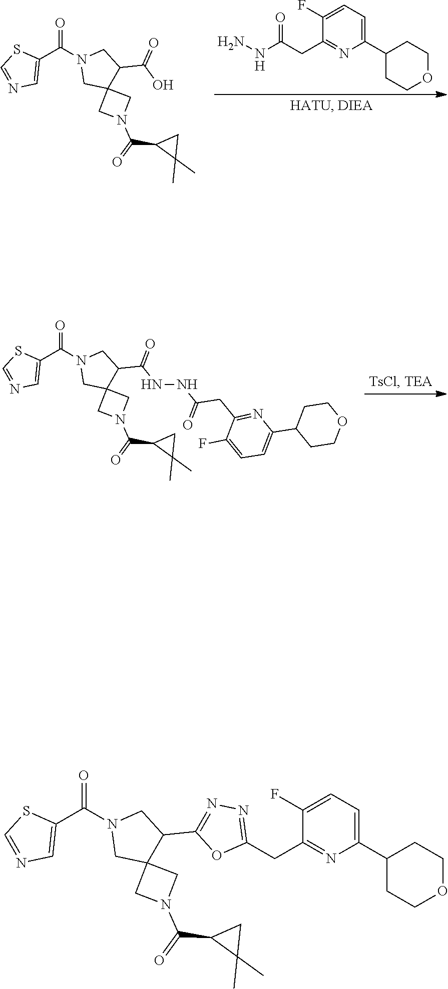 [SMILES: CC1(C)C[C@@H]1C(=O)N1CC2(CN(C(=O)c3cncs3)CC2C(=O)O)C1 ; NNC(=O)Cc1nc(C2CCOCC2)ccc1F ; CC1(C)C[C@@H]1C(=O)N1CC2(CN(C(=O)c3cncs3)CC2C(=O)NNC(=O)Cc2nc(C3CCOCC3)ccc2F)C1 ; CC1(C)C[C@@H]1C(=O)N1CC2(CN(C(=O)c3cncs3)CC2c2nnc(Cc3nc(C4CCOCC4)ccc3F)o2)C1]

### Step 1: 2-((S)-2,2-dimethylcyclopropane-1-carbonyl)-N′-(2-(3-fluoro-6-(tetrahydro-2H-pyran-4-yl)pyridin-2-yl)acetyl)-6-(thiazole-5-carbonyl)-2,6-diazaspiro[3.4]octane-8-carbohydrazide

To a solution of 2-((S)-2,2-dimethylcyclopropane-1-carbonyl)-6-(thiazole-5-carbonyl)-2,6-diazaspiro[3.4]octane-8-carboxylic acid (60 mg, 0.16 mmol) in DCM (1 mL) was added HATU (63 mg, 0.16 mmol) and DIPEA (64 mg, 0.49 mmol). The mixture was stirred at room temperature for 30 min then 2-(3-fluoro-6-(tetrahydro-2H-pyran-4-yl)pyridin-2-yl)acetohydrazide (42 mg, 0.16 mmol) was added and stirring continued for another 2 h. The mixture was diluted with water (15 mL) and extracted with DCM (30 mL×3). The combined organic layers were washed with brine, dried over Na₂SO₄, filtered and concentrated. The residue was purified by prep-TLC (eluent: DCM:MeOH=20:1, v/v) to afford 2-((S)-2,2-dimethylcyclopropane-1-carbonyl)-N′-(2-(3-fluoro-6-(tetrahydro-2H-pyran-4-yl)pyridin-2-yl)acetyl)-6-(thiazole-5-carbonyl)-2,6-diazaspiro[3.4]octane-8-carbohydrazide (54 mg, 55%) as a white solid. LCMS m/z=599.0 [M+H]⁺; ¹H NMR (400 MHz, Chloroform-d) δ 8.91 (s, 1H), 8.26 (s, 1H), 7.79-7.57 (m, 1H), 7.40-7.29 (m, 1H), 5.30 (s, 2H), 4.26-4.00 (m, 12H), 3.37 (s, 1H), 3.32-3.21 (m, 1H), 2.34-2.21 (m, 4H), 1.31-1.27 (m, 1H), 1.12 (s, 6H), 1.08-1.06 (m, 1H), 0.77-0.70 (m, 1H).

### Step 7: (2-((S)-2,2-dimethylcyclopropane-1-carbonyl)-8-(5-((3-fluoro-6-(tetrahydro-2H-pyran-4-yl)pyridin-2-yl)methyl)-1,3,4-oxadiazol-2-yl)-2,6-diazaspiro[3.4]octan-6-yl)(thiazol-5-yl)methanone

To a solution of 2-((S)-2,2-dimethylcyclopropane-1-carbonyl)-N′-(2-(3-fluoro-6-(tetrahydro-2H-pyran-4-yl)pyridin-2-yl)acetyl)-6-(thiazole-5-carbonyl)-2,6-diazaspiro[3.4]octane-8-carbohydrazide (40 mg, 0.066 mmol) in DCM (1 mL) was added triethylamine (21 mg, 0.20 mmol) and TsCl (38 mg, 0.20 mmol). The reaction mixture was stirred at room temperature for 2 h then the solvent was removed under reduced pressure. The residue obtained was purified by prep-HPLC to afford (2-((S)-2,2-dimethylcyclopropane-1-carbonyl)-8-(5-((3-fluoro-6-(tetrahydro-2H-pyran-4-yl)pyridin-2-yl)methyl)-1,3,4-oxadiazol-2-yl)-2,6-diazaspiro[3.4]octan-6-yl)(thiazol-5-yl)methanone (10 mg, 26%) as a white solid. LCMS m/z=581.3 [M+H]⁺; ¹H NMR (400 MHz, DMSO-d₆) δ 9.31-9.22 (m, 1H), 8.43-8.34 (m, 1H), 7.71-7.61 (m, 1H), 7.34-7.28 (m, 1H), 4.54-4.41 (m, 2H), 4.34-4.04 (m, 5H), 3.99-3.79 (m, 6H), 3.41 (s, 2H), 2.92-2.82 (m, 1H), 1.72-1.60 (m, 4H), 1.36-1.23 (m, 1H), 1.12-0.94 (m, 6H), 0.89-0.81 (m, 1H), 0.73-0.60 (m, 1H).

Table 11: The compounds listed in Table 11 were synthesized from 2-((S)-2,2-dimethylcyclopropane-1-carbonyl)-6-(thiazole-5-carbonyl)-2,6-diazaspiro[3.4]octane-8-carboxylic acid according to the procedures outlined for I-50 using the appropriate commercially available reagents and/or intermediates described elsewhere.

TABLE 11

| Example | | | |
|---|---|---|---|
| Number | Compound | ¹HNMR | LCMS |
| I-55 | 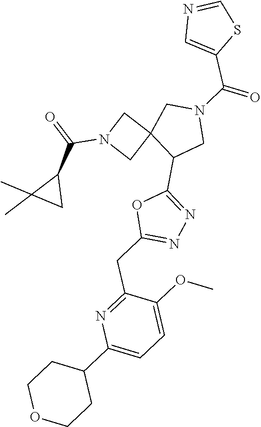 [SMILES: COc1ccc(C2CCOCC2)nc1Cc1nnc(C2CN(C(=O)c3cncs3)CC23CN(C(=O)[C@H]2CC2(C)C)C3)o1] | ¹H NMR (400 MHz, DMSO-d₆) δ 9.27 (s, 1H), 8.41-8.26 (m, 1H), 7.41-7.36 (m, 1H), 7.18 (d, J = 8.5 Hz, 1H), 4.38- 4.00 (m, 8H), 3.95- 3.85 (m, 4H), 3.78 (s, 4H), 3.41-3.38 (m, 2H), 2.83-2.72 (m, 1H), 1.69- 1.55 (m, 4H), 1.37- 1.21 (m, 1H), 1.14-1.08 (m, 2H), 1.07-1.03 (m, 2H), 1.01 (s, 1H), 0.96 (s, 1H), 0.88-0.82 (m, 1H), 0.71-0.64 (m, 1H). | m/z = 592.3 [M + H]⁺ |
| I-97 | 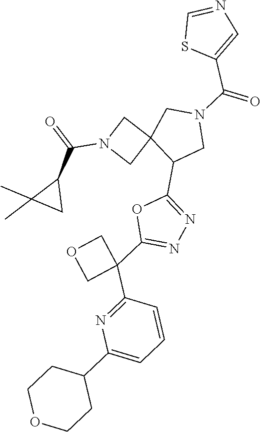 [SMILES: CC1(C)C[C@@H]1C(=O)N1CC2(CN(C(=O)c3cncs3)CC2c2nnc(C3(c4cccc(C5CCOCC5)n4)COC3)o2)C1] | ¹H NMR (400 MHz, Chloroform-d) δ 8.93 (s, 1H), 8.30-8.22 (m, 1H), 7.73-7.62 (m, 1H), 7.17- 7.10 (m, 1H), 7.10- 6.97 (m, 1H), 5.42-5.26 (m, 4H), 4.33-4.22 (m, 3H), 4.15-4.00 (m, 7H), 3.95-3.76 (m, 2H), 3.53 (dt, J = 11.4, 5.6 Hz, 2H), 3.00-2.88 (m, 1H), 1.93-1.81 (m, 4H), 1.19- 1.08 (m, 8H), 0.80- 0.71 (m, 1H). | m/z = 605.3 [M + H]⁺ |
| I-83 | 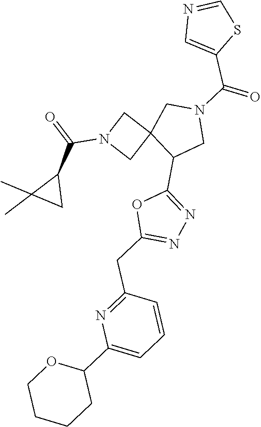 [SMILES: CC1(C)C[C@@H]1C(=O)N1CC2(CN(C(=O)c3cncs3)CC2c2nnc(Cc3cccc(C4CCCCO4)n3)o2)C1] | ¹H NMR (400 MHz, Chloroform-d) δ 8.93 (s, 1H), 8.25 (d, J = 3.3 Hz, 1H), 7.67 (d, J = 7.8 Hz, 1H), 7.39 (d, J = 7.8 Hz, 1H), 7.13 (s, 1H), 4.41 (s, 3H), 4.27 (d, J = 9.0 Hz, 2H), 4.18-3.94 (m, 7H), 3.80 (s, 1H), 3.64 (d, J = 11.4 Hz, 1H), 2.05-1.86 (m, 3H), 1.68 (s, 3H), 1.13 (d, J = 7.5 Hz, 8H), 0.76 (d, J = 7.6 Hz, 1H) | m/z = 563.3 [M + H]⁺ |
| I-25 | 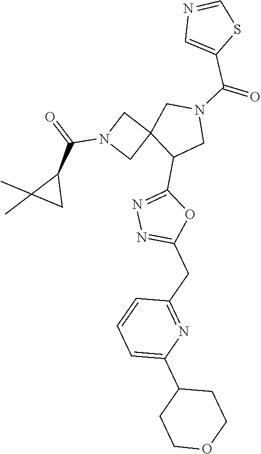 [SMILES: CC1(C)C[C@@H]1C(=O)N1CC2(CN(C(=O)c3cncs3)CC2c2nnc(Cc3cccc(C4CCOCC4)n3)o2)C1] | ¹H NMR (400 MHz, CD₃OD) δ 9.17 (s, 1H), 8.37 (d, J = 5.6 Hz, 1H), 7.72 (t, J = 7.8 Hz, 1H), 7.22 (t, J = 7.6 Hz, 2H), 4.59-4.17 (m, 6H), 4.16-3.87 (m, 7H), 3.54 (td, J = 11.4, 3.4 Hz, 2H), 2.92 (t, J = 5.4 Hz, 1H), 1.89-1.67 (m, 4H), 1.39 (s, 1H), 1.21-1.00 (m, 7H), 0.77 (m, 1H). | m/z = 563.4 [M + H]⁺ |
| I-33 | 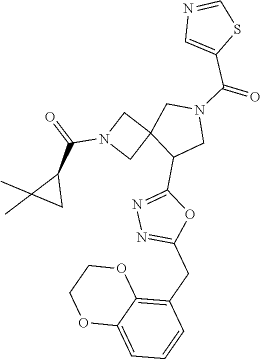 [SMILES: CC1(C)C[C@@H]1C(=O)N1CC2(CN(C(=O)c3cncs3)CC2c2nnc(Cc3cccc4c3OCCO4)o2)C1] | ¹H NMR (400 MHz, CD₃OD) δ 9.16 (s, 1H), 8.36 (s, 1H), 6.76 (s, 3H), 4.47-3.80 (m, 15H), 1.49-0.95 (m, 8H), 0.81- 0.69 (m, 1H). | m/z = 536.3 [M + H]⁺ |
| I-26 | 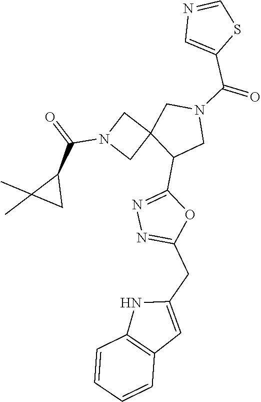 [SMILES: CC1(C)C[C@@H]1C(=O)N1CC2(CN(C(=O)c3cncs3)CC2c2nnc(Cc3cc4ccccc4[nH]3)o2)C1] | ¹H NMR (400 MHz, Methanol-d4) δ 9.14 (s, 1H), 8.37-8.18 (m, 1H), 7.58-7.42 (m, 1H), 7.34 (d, J = 8.0 Hz, 1H), 7.22 (d, J = 10.2 Hz, 1H), 7.09 (t, J = 7.6 Hz, 1H), 7.03- 6.94 (m, 1H), 4.44-3.81 (m, 9H), 1.36-1.26 (m, 1H), 1.18-1.01 (m, 6H), 0.78-0.66 (m, 1H). | m/z = 517.3 [M + H]⁺ |
| I-32 | 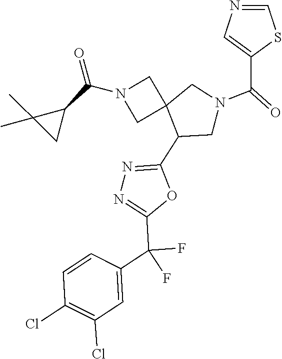 [SMILES: CC1(C)C[C@@H]1C(=O)N1CC2(CN(C(=O)c3cncs3)CC2c2nnc(C(F)(F)c3ccc(Cl)c(Cl)c3)o2)C1] | ¹H NMR (400 MHz, CD₃OD) δ 9.16 (s, 1H), 8.37 (d, J = 5.4 Hz, 1H), 7.87 (d, J = 6.6 Hz, 1H), 7.78-7.68 (m, 1H), 7.61 (d, J = 7.2 Hz, 1H), 4.54- 3.90 (m, 9H), 1.46- 1.31 (m, 1H), 1.23-0.83 (m, 7H), 0.81-0.70 (m, 1H). | m/z = 582.2 [M + H]⁺ |
| I-27 | 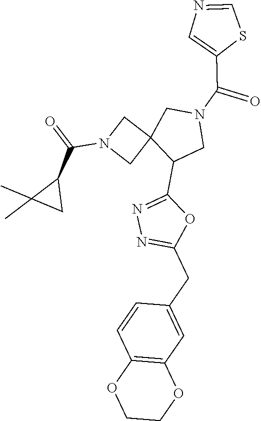 [SMILES: CC1(C)C[C@@H]1C(=O)N1CC2(CN(C(=O)c3cncs3)CC2c2nnc(Cc3ccc4c(c3)OCCO4)o2)C1] | ¹H NMR (400 MHz, CD₃OD) δ 9.17 (s, 1H), 8.37 (d, J = 5.5 Hz, 1H), 6.81-6.68 (m, 3H), 4.50- 3.85 (m, 16H), 1.40- 1.24 (m, 1H), 1.20-1.14 (m, 2H), 1.13-0.91 (m, 5H), 0.82-0.71 (m, 1H). | m/z = 536.4 [M + H]⁺ |
| I-71 | 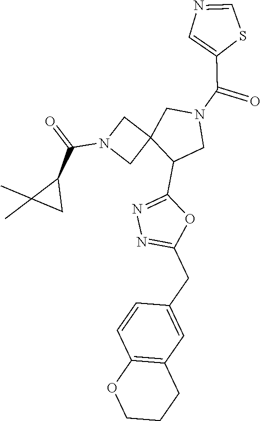 [SMILES: CC1(C)C[C@@H]1C(=O)N1CC2(CN(C(=O)c3cncs3)CC2c2nnc(Cc3ccc4c(c3)CCCO4)o2)C1] | ¹H NMR (400 MHz, CD₃OD) δ 9.16 (s, 1H), 8.37 (s, 1H), 6.95 (s, 2H), 6.69-6.59 (m, 1H), 4.50- 3.83 (m, 13H), 2.72 (t, J = 6.6 Hz, 2H), 1.97- 1.88 (m, 2H), 1.49-0.94 (m, 8H), 0.80-0.70 (m, 1H). | m/z = 534.3 [M + H]⁺ |
| I-28 | 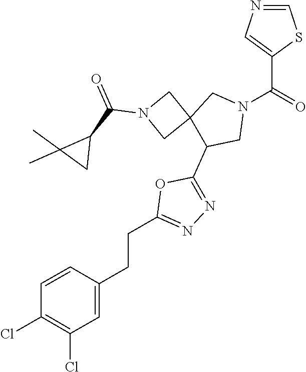 [SMILES: CC1(C)C[C@@H]1C(=O)N1CC2(CN(C(=O)c3cncs3)CC2c2nnc(CCc3ccc(Cl)c(Cl)c3)o2)C1] | ¹H NMR (400 MHz, CD₃OD). δ 9.17 (s, 1H), 8.39 (d, J = 4.6 Hz, 1H), 7.46-7.39 (m, 2H), 7.20-7.10 (m, 1H), 4.56- 4.19 (m, 4H), 4.18- 3.91 (m, 5H), 3.26-3.16 (m, 2H), 3.14-3.04 (m, 2H), 1.50-1.30 (m, 2H), 1.23-0.90 (m, 7H), 0.82- 0.73 (m, 1H). | m/z = 560.2 [M + H]⁺ |
| I-65 | 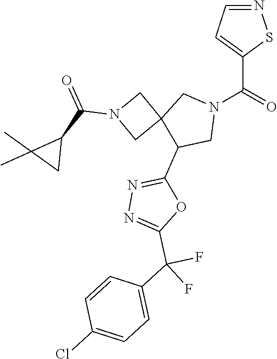 [SMILES: CC1(C)C[C@@H]1C(=O)N1CC2(CN(C(=O)c3ccns3)CC2c2nnc(C(F)(F)c3ccc(Cl)cc3)o2)C1] | ¹H NMR (400 MHz, CD₃OD) δ 9.16 (s, 1H), 8.37 (d, J = 5.2 Hz, 1H), 7.66 (d, J = 7.2 Hz, 2H), 7.61-7.51 (m, 2H), 4.56- 3.84 (m, 9H), 1.46- 1.31 (m, 1H), 1.21-0.98 (m, 7H), 0.83-0.67 (m, 1H). | m/z = 548.2 [M + H]⁺ |
| I-70 | 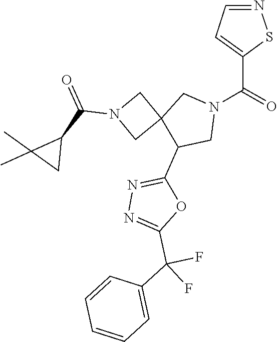 [SMILES: CC1(C)C[C@@H]1C(=O)N1CC2(CN(C(=O)c3ccns3)CC2c2nnc(C(F)(F)c3ccccc3)o2)C1] | ¹H NMR (400 MHz, CD₃OD) ¹H δ 9.17 (s, 1H), 8.38 (d, J = 4.6 Hz, 1H), 7.74-7.49 (m, 5H), 4.58-4.22 (m, 4H), 4.21- 3.98 (m, 5H), 3.97- 3.90 (m, 1H), 1.38-1.32 (m, 1H), 1.22-1.06 (m, 6H), 1.04-0.98 (m, 2H), 0.82-0.72 (m, 1H). | m/z = 514.3 [M + H]⁺ |
| I-34 | 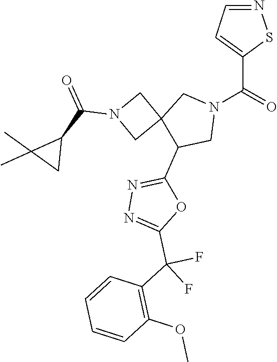 [SMILES: COc1ccccc1C(F)(F)c1nnc(C2CN(C(=O)c3ccns3)CC23CN(C(=O)[C@H]2CC2(C)C)C3)o1] | ¹H NMR (400 MHz, CD₃OD) δ 9.16 (s, 1H), 8.37 (s, 1H), 7.70 (d, J = 7.3 Hz, 1H), 7.57 (t, J = 8.1 Hz, 1H), 7.19-7.06 (m, 2H), 4.61-3.82 (m, 10H), 3.74-3.62 (m, 3H), 1.36-1.29 (m, 1H), 1.21-0.98 (m, 7H), 0.82- 0.74 (m, 1H). | m/z = 544.3 [M + H]⁺ |
| I-76 | 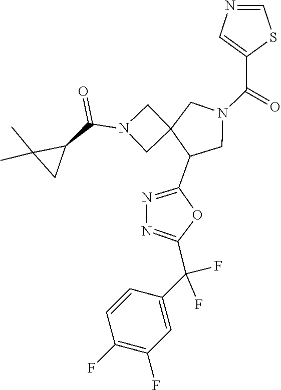 [SMILES: CC1(C)C[C@@H]1C(=O)N1CC2(CN(C(=O)c3cncs3)CC2c2nnc(C(F)(F)c3ccc(F)c(F)c3)o2)C1] | ¹H NMR (400 MHz, CD₃OD) δ 9.16 (s, 1H), 8.43-8.34 (m, 1H), 7.68 (d, J = 9.2 Hz, 1H), 7.61- 7.40 (m, 2H), 4.60- 3.92 (m, 9H), 1.38 (s, 1H), 1.22-0.98 (m, 7H), 0.80-0.70 (m, 1H). | m/z = 550.2 [M + H]⁺ |
| I-61 | 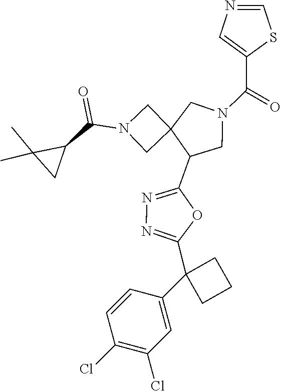 | ¹H NMR (400 MHz, DMSO-d6) δ 9.26 (d, J = 3.0 Hz, 1H), 8.38 (d, J = 15.2 Hz, 1H), 7.73-7.56 (m, 2H), 7.45-7.33 (m, 1H), 5.44 (s, 1H), 4.36- 3.57 (m, 9H), 3.11-2.95 (m, 2H), 2.80-2.61 (m, 2H), 1.41-1.20 (m, 1H), 1.18-0.89 (m, 6H), 0.88- 0.78 (m, 1H), 0.73- 0.60 (m, 1H). | m/z = 602.1 [M + H]⁺ |
| I-39 | 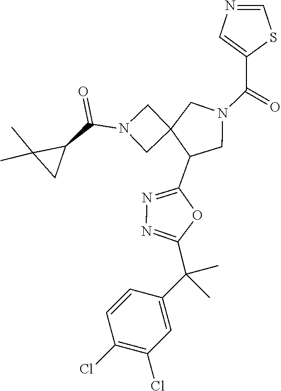 [SMILES: CC(C)(c1ccc(Cl)c(Cl)c1)c1nnc(C2CN(C(=O)c3cncs3)CC23CN(C(=O)[C@H]2CC2(C)C)C3)o1] | ¹H NMR (400 MHz, Methanol-d₄) δ 9.16 (s, 1H), 8.36 (d, J = 9.2 Hz, 1H), 7.48 (t, J = 7.2 Hz, 2H), 7.30-7.10 (m, 1H), 4.48-3.85 (m, 9H), 1.79 (d, J = 4.0 Hz, 6H), 1.46- 1.26 (m, 1H), 1.17 (d, J = 8.8 Hz, 2H), 1.15- 1.00 (m, 5H), 0.77 (s, 1H). | m/z = 574.2 [M + H]⁺ |
| I-31 | 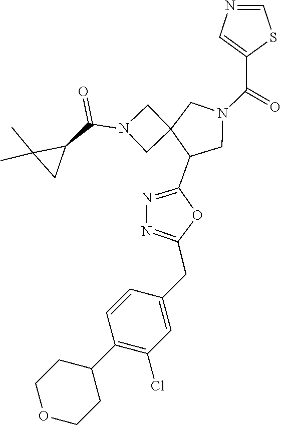 [SMILES: CC1(C)C[C@@H]1C(=O)N1CC2(CN(C(=O)c3cncs3)CC2c2nnc(Cc3ccc(C4CCOCC4)c(Cl)c3)o2)C1] | ¹H NMR (400 MHz, CD3OD) δ 9.16 (s, 1H), 8.37 (d, J = 6.5 Hz, 1H), 7.38 (d, J = 6.3 Hz, 1H), 7.32 (dd, J = 8.1, 1.7 Hz, 1H), 7.24 (d, J = 7.7 Hz, 1H), 4.39-3.89 (m, 13H), 3.68-3.53 (m, 3H), 3.26 (q, J = 9.1, 7.0 Hz, 1H), 1.75 (td, J = 9.3, 7.9, 3.5 Hz, 4H), 1.44- 1.32 (m, 1H), 1.22-0.88 (m, 8H), 0.75 (s, 1H). | m/z = 596.3 [M + H]⁺ |
| I-40 | 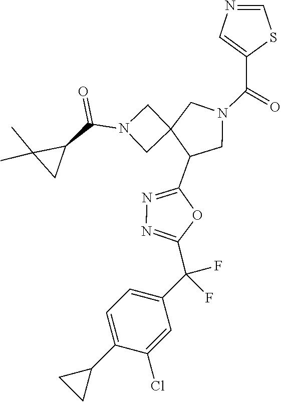 | ¹H NMR (400 MHz, CD₃OD) δ 9.16 (s, 1H), 8.37 (d, J = 5.7 Hz, 1H), 7.49 (d, J = 7.6 Hz, 2H), 7.25-7.19 (m, 2H), 4.47- 3.92 (m, 9H), 1.98 (m, J = 8.6, 5.2 Hz, 1H), 1.45- 1.30 (m, 1H), 1.20- 0.98 (m, 9H), 0.75 (dt, J = 6.7, 4.6 Hz, 3H). | m/z = 554.2 [M + H]⁺ |
| I-19 | 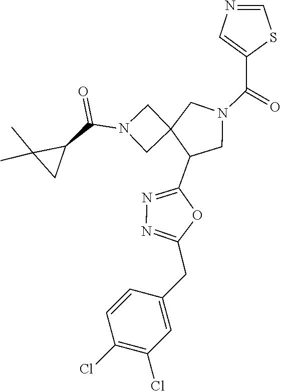 [SMILES: CC1(C)C[C@@H]1C(=O)N1CC2(CN(C(=O)c3cncs3)CC2c2nnc(Cc3ccc(Cl)c(Cl)c3)o2)C1] | ¹H NMR (400 MHz, CD₃OD) δ 9.16 (s, 1H), 8.42-8.29 (m, 1H), 7.59- 7.43 (m, 2H), 7.29- 7.19 (m, 1H), 4.44-3.92 (m, 12H), 1.40-1.34 (m, 2H), 1.18-0.99 (m, 6H), 0.80-0.69 (m, 1H). | m/z = 546.2 [M + H]⁺ |
| I-78 | 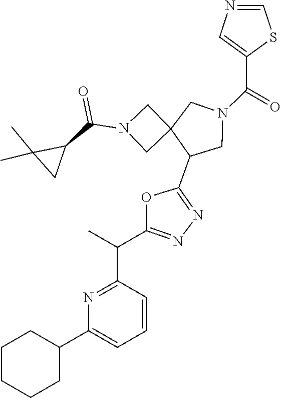 [SMILES: CC(c1cccc(C2CCCCC2)n1)c1nnc(C2CN(C(=O)c3cncs3)CC23CN(C(=O)[C@H]2CC2(C)C)C3)o1] | ¹H NMR (400 MHz, CD₃OD) δ 9.16 (s, 1H), 8.36 (d, J = 8.3 Hz, 1H), 7.75-7.65 (m, 1H), 7.23- 7.14 (m, 2H), 4.64-3.91 (m, 11H), 3.58-3.48 (m, 2H), 2.90 (s, 1H), 1.80- 1.73 (m, 6H), 1.41-1.37 (m, 2H), 1.20-1.00 (m, 8H), 0.76 (s, 1H). | m/z = 577.4 [M + H]⁺ |
| I-30 | 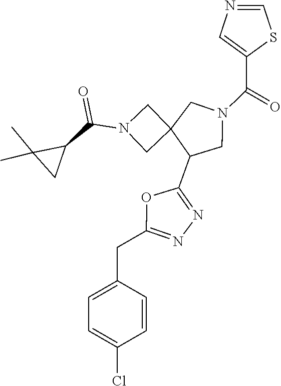 [SMILES: CC1(C)C[C@@H]1C(=O)N1CC2(CN(C(=O)c3cncs3)CC2c2nnc(Cc3ccc(Cl)cc3)o2)C1] | ¹H NMR (400 MHz, Methanol-d₄) δ 9.17 (s, 1H), 8.37 (d, J = 5.6 Hz, 1H), 7.35-7.29 (m, 4H), 4.39-3.88 (m, 12H), 1.31-1.29 (m, 1H), 1.20- 1.05 (m, 6H), 0.98 (s, 1H). | m/z = 512.3 [M + H]⁺ |
| I-72 | 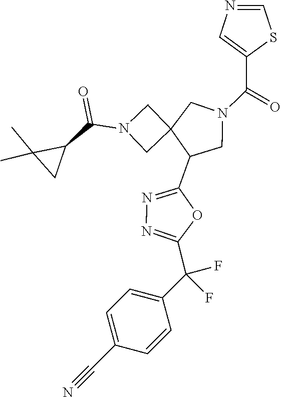 [SMILES: CC1(C)C[C@@H]1C(=O)N1CC2(CN(C(=O)c3cncs3)CC2c2nnc(C(F)(F)c3ccc(C#N)cc3)o2)C1] | ¹H NMR (400 MHz, CD₃OD) δ 9.17 (s, 1H), 8.42-8.34 (m, 1H), 8.01- 7.78 (m, 4H), 4.52-3.93 (m, 9H), 1.45-1.33 (m, 1H), 1.20-1.00 (m, 7H), 0.77 (s, 1H). | m/z = 539.3 [M + H]⁺ |
| I-73 | 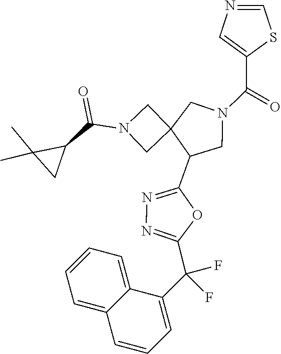 [SMILES: CC1(C)C[C@@H]1C(=O)N1CC2(CN(C(=O)c3cncs3)CC2c2nnc(C(F)(F)c3cccc4ccccc34)o2)C1] | ¹H NMR (400 MHz, Methanol-d₄) δ 9.16 (s, 1H), 8.31 (s, 1H), 8.12 (d, J = 8.0 Hz, 1H), 8.02- 7.87 (m, 3H), 7.64- 7.50 (m, 3H), 4.35-3.83 (m, 9H), 1.33 (s, 1H), 1.17-1.00 (m, 7H), 0.79-0.73 (m, 1H). | m/z = 564.3 [M + H]⁺ |
| I-96 | 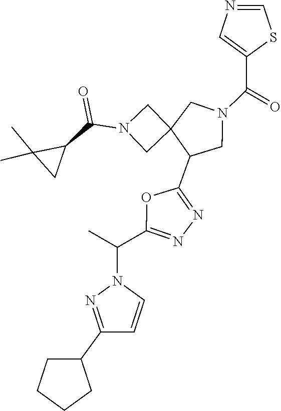 [SMILES: CC(c1nnc(C2CN(C(=O)c3cncs3)CC23CN(C(=O)[C@H]2CC2(C)C)C3)o1)n1ccc(C2CCCC2)n1] | ¹H NMR (400 MHz, CD₃OD) δ 9.17 (s, 1H), 8.44-8.31 (m, 1H), 7.66 (d, J = 4.9 Hz, 1H), 6.18- 6.08 (m, 1H), 5.83 (s, 1H), 4.59-3.90 (m, 10H), 3.01 (d, J = 9.0 Hz, 1H), 2.08-1.89 (m, 5H), 1.82-1.55 (m, 6H), 1.45- 1.36 (m, 1H), 1.21- 1.02 (m, 7H), 0.78 (s, 1H). | m/z = 550.3 [M + H]⁺ |
| I-67 | 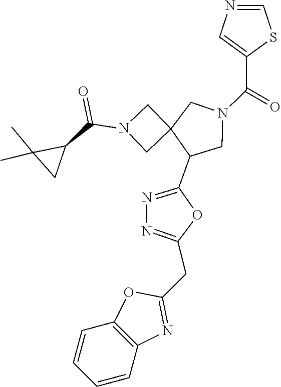 [SMILES: CC1(C)C[C@@H]1C(=O)N1CC2(CN(C(=O)c3cncs3)CC2c2nnc(Cc3nc4ccccc4o3)o2)C1] | ¹H NMR (400 MHz, CD₃OD) 1H NMR (400 MHz, Methanol-d4) δ 9.15 (d, J = 3.1 Hz, 1H), 8.38 (d, J = 5.4 Hz, 1H), 7.66 (d, J = 6.5 Hz, 1H), 7.63-7.40 (m, 3H), 4.67- 3.51 (m, 10H), 1.29- 0.86 (m, 8H), 0.73 (dt, J = 9.6, 4.6 Hz, 1H). | m/z = 519.3 [M + H]⁺ |
| I-69 | 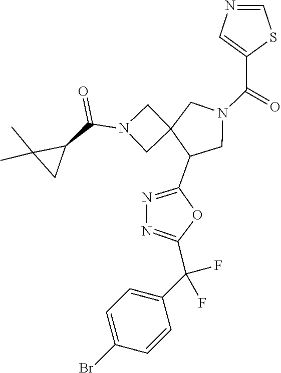 [SMILES: CC1(C)C[C@@H]1C(=O)N1CC2(CN(C(=O)c3cncs3)CC2c2nnc(C(F)(F)c3ccc(Br)cc3)o2)C1] | ¹H NMR (400 MHz, CD₃OD) δ 8.36 (s, 1H), 7.58 (s, 1H), 6.96-6.88 (m, 2H), 6.78 (s, 2H), 3.74-3.19 (m, 10H), 0.57 (s, 1H), 0.39-0.17 (m, 8H). | m/z = 592.2 [M + H]⁺ |
| I-68 | 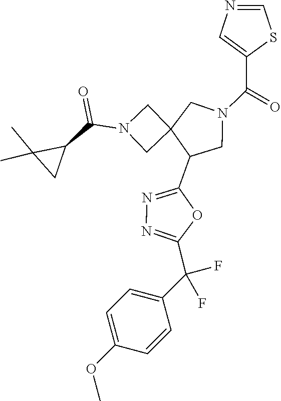 [SMILES: COc1ccc(C(F)(F)c2nnc(C3CN(C(=O)c4cncs4)CC34CN(C(=O)[C@H]3CC3(C)C)C4)o2)cc1] | ¹H NMR (400 MHz, CD₃OD) δ 9.16 (s, 1H), 8.37-7.55 (m, Hz, 3H), 7.21-6.96 (m, 2H), 4.46- 3.94 (m, 9H), 3.85 (s, 3H), 1.36-0.76 (m, 9H). | m/z = 544.3 [M + H]⁺ |

### Synthesis of (8-(5-((1H-pyrazol-3-yl)methyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone (I-29)

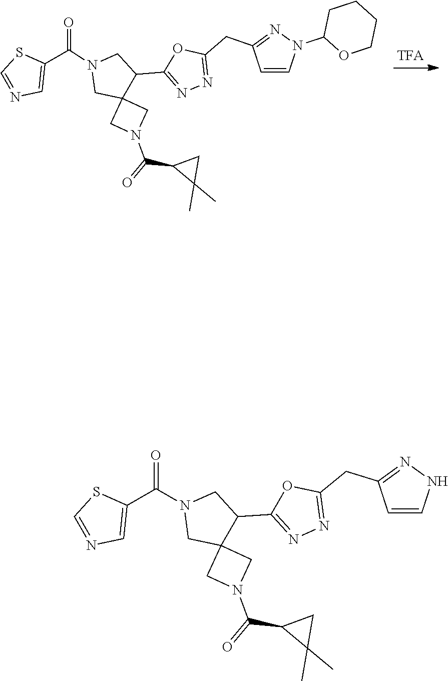 [SMILES: CC1(C)C[C@@H]1C(=O)N1CC2(CN(C(=O)c3cncs3)CC2c2nnc(Cc3ccn(C4CCCCO4)n3)o2)C1 ; CC1(C)C[C@@H]1C(=O)N1CC2(CN(C(=O)c3cncs3)CC2c2nnc(Cc3cc[nH]n3)o2)C1]

### (2-((S)-2,2-dimethylcyclopropane-1-carbonyl)-8-(5-((1-(tetrahydro-2H-pyran-2-yl)-1H-pyrazol-3-yl)methyl)-1,3,4-oxadiazol-2-yl)-2,6-diazaspiro[3.4]octan-6-yl)(thiazol-5-yl)methanone

(2-((S)-2,2-dimethylcyclopropane-1-carbonyl)-8-(5-((1-(tetrahydro-2H-pyran-2-yl)-1H-pyrazol-3-yl)methyl)-1,3,4-oxadiazol-2-yl)-2,6-diazaspiro[3.4]octan-6-yl)(thiazol-5-yl)methanone was synthesized from 2-((S)-2,2-dimethylcyclopropane-1-carbonyl)-6-(thiazole-5-carbonyl)-2,6-diazaspiro[3.4]octane-8-carboxylic acid according to the procedures outlined for I-50 using the appropriate commercially available reagents and/or intermediates described elsewhere. LCMS m/z=552.1 [M+H]⁺.

### Step 1: (8-(5-((1H-pyrazol-3-yl)methyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone

To a solution of (2-((S)-2,2-dimethylcyclopropane-1-carbonyl)-8-(5-((1-(tetrahydro-2H-pyran-2-yl)-1H-pyrazol-3-yl)methyl)-1,3,4-oxadiazol-2-yl)-2,6-diazaspiro[3.4]octan-6-yl)(thiazol-5-yl)methanone (25 mg, 0.045 mmol) in DCM (3 mL) was added TFA (0.5 mL). The reaction was stirred for 2 hours then concentrated in vacuo. The residue obtained was purified by prep-TLC (eluent: DCM:MeOH=15:1, v/v) to afford (8-(5-((1H-pyrazol-3-yl)methyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone (8.4 mg, 40%) as a white solid. LCMS m/z=468.2 [M+H]⁺; ¹H NMR (400 MHz, CD₃OD) δ 9.16 (s, 1H), 8.38 (s, 1H), 7.61 (s, 1H), 6.27 (s, 1H), 4.59-4.20 (m, 6H), 4.19-3.89 (m, 5H), 1.44-1.33 (m, 2H), 1.21-1.07 (m, 5H), 1.06-0.89 (m, 2H), 0.81-0.70 (m, 1H).

### Synthesis of (1-benzyl-1H-pyrazol-4-yl)(2-((S)-2,2-dimethylcyclopropane-1-carbonyl)-8-(5-(pyrimidin-5-ylmethyl)-1,3,4-oxadiazol-2-yl)-2,6-diazaspiro[3.4]octan-6-yl)methanone (I-14)

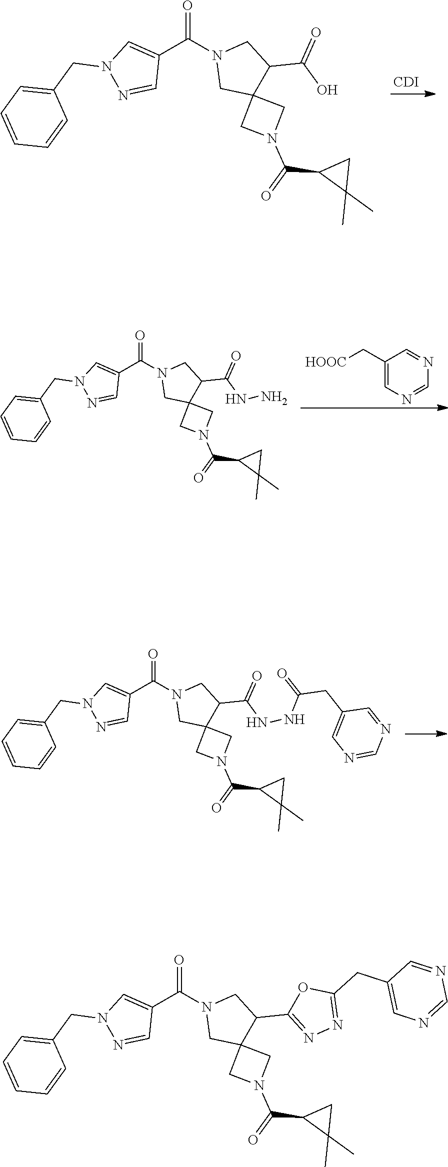 [SMILES: CC1(C)C[C@@H]1C(=O)N1CC2(CN(C(=O)c3cnn(Cc4ccccc4)c3)CC2C(=O)O)C1 ; "CDI" ; CC1(C)C[C@@H]1C(=O)N1CC2(CN(C(=O)c3cnn(Cc4ccccc4)c3)CC2C(=O)NN)C1 ; O=C(O)Cc1cncnc1 ; CC1(C)C[C@@H]1C(=O)N1CC2(CN(C(=O)c3cnn(Cc4ccccc4)c3)CC2C(=O)NNC(=O)Cc2cncnc2)C1 ; CC1(C)C[C@@H]1C(=O)N1CC2(CN(C(=O)c3cnn(Cc4ccccc4)c3)CC2c2nnc(Cc3cncnc3)o2)C1]

### Step 1: 6-(1-benzyl-1H-pyrazole-4-carbonyl)-2-((S)-2,2-dimethylcyclopropane-1-carbonyl)-2,6-diazaspiro[3.4]octane-8-carbohydrazide

To a solution of 6-(1-benzyl-1H-pyrazole-4-carbonyl)-2-((S)-2,2-dimethylcyclopropane-1-carbonyl)-2,6-diazaspiro[3.4]octane-8-carboxylic acid (1.0 g, 2.29 mmol) in THF (20 mL) at 0° C. was added CDI (0.45 g, 2.75 mmol). The reaction was stirred for 1 h, then hydrazine hydrate (98%, 0.34 g, 6.86 mmol) was added dropwise. The reaction was allowed to warm to room temperature and stirred a further 14 h, then diluted with water (80 mL) and extracted with 20% MeOH/DCM (50 mL×4). The combined organic layers were dried over Na₂SO₄, filtered and concentrated to afford 6-(1-benzyl-1H-pyrazole-4-carbonyl)-2-((S)-2,2-dimethylcyclopropane-1-carbonyl)-2,6-diazaspiro[3.4]octane-8-carbohydrazide (0.7 g, 70%) as a white solid. LCMS m/z=451.3 [M+H]⁺.

### Step 2: 6-(1-benzyl-1H-pyrazole-4-carbonyl)-2-((S)-2,2-dimethylcyclopropane-1-carbonyl)-N′-(2-(pyrimidin-5-yl)acetyl)-2,6-diazaspiro[3.4]octane-8-carbohydrazide

To a solution of 6-(1-benzyl-1H-pyrazole-4-carbonyl)-2-((S)-2,2-dimethylcyclopropane-1-carbonyl)-2,6-diazaspiro[3.4]octane-8-carbohydrazide (150 mg, 0.33 mmol) in DMF (2 mL) was added 2-(pyrimidin-5-yl)acetic acid (55.2 mg, 0.40 mmol), EDCI (96 mg, 0.50 mmol), HOBt (67 mg, 0.50 mmol) and DIPEA (129 mg, 1.0 mmol). The resulting mixture was stirred at room temperature for 14 h then the solvent was removed under reduced pressure. The residue obtained was purified by prep-TLC (DCM:MeOH=15:1) to afford 6-(1-benzyl-1H-pyrazole-4-carbonyl)-2-((S)-2,2-dimethylcyclopropane-1-carbonyl)-N′-(2-(pyrimidin-5-yl)acetyl)-2,6-diazaspiro[3.4]octane-8-carbohydrazide (180 mg, 94%) as a yellow solid. LCMS m/z=569.2 [M+H]⁺.

### Step 3: (1-benzyl-1H-pyrazol-4-yl)(2-((S)-2,2-dimethylcyclopropane-1-carbonyl)-8-(5-(pyrimidin-5-ylmethyl)-1,3,4-oxadiazol-2-yl)-2,6-diazaspiro[3.4]octan-6-yl)methanone

To a solution of 6-(1-benzyl-1H-pyrazole-4-carbonyl)-2-((S)-2,2-dimethylcyclopropane-1-carbonyl)-N′-(2-(pyrimidin-5-yl)acetyl)-2,6-diazaspiro[3.4]octane-8-carbohydrazide (180 mg, 0.32 mmol) in DCM (5 mL) was added TEA (32 mg, 0.32 mmol), TsCl (60.1 mg, 0.32 mmol). The resulting mixture was stirred at room temperature for 14 h then the solvent was removed under reduced pressure. The residue obtained was purified by prep-TLC (DCM:MeOH=10:1) to afford (1-benzyl-1H-pyrazol-4-yl)(2-((S)-2,2-dimethylcyclopropane-1-carbonyl)-8-(5-(pyrimidin-5-ylmethyl)-1,3,4-oxadiazol-2-yl)-2,6-diazaspiro[3.4]octan-6-yl)methanone (50 mg, 28%) as a white solid. LCMS m/z=553.4 [M+H]⁺; 1H NMR (400 MHz, Methanol-d4) δ 9.08 (s, 1H), 8.82 (d, J=6.0 Hz, 2H), 8.22 (d, J=12.8 Hz, 1H), 7.92 (d, J=8.8 Hz, 1H), 7.41-7.23 (m, 5H), 5.38 (s, 2H), 4.48-3.87 (m, 11H), 1.46-1.33 (m, 1H), 1.20-0.99 (m, 7H), 0.82-0.70 (m, 1H).

Table 12: The compounds listed in Table 12 were synthesized from 6-(1-benzyl-1H-pyrazole-4-carbonyl)-2-((S)-2,2-dimethylcyclopropane-1-carbonyl)-2,6-diazaspiro[3.4]octane-8-carbohydrazide according to the procedures outlined for I-14 using the appropriate commercially available reagents and/or intermediates described elsewhere.

TABLE 12

| Example | | | |
|---|---|---|---|
| Number | Compound | ¹HNMR | LCMS |
| I-23 | 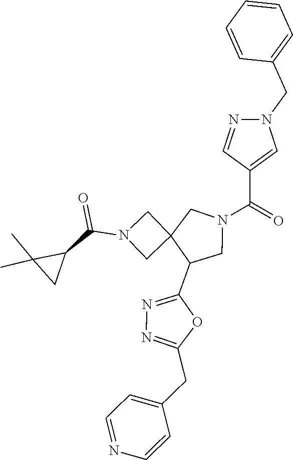 [SMILES: CC1(C)C[C@@H]1C(=O)N1CC2(CN(C(=O)c3cnn(Cc4ccccc4)c3)CC2c2nnc(Cc3ccncc3)o2)C1] | ¹H NMR (400 MHz, Methanol-d₄) δ 8.63- 8.33 (m, 2H), 8.22 (d, J = 11.0 Hz, 1H), 7.92 (d, J = 9.0 Hz, 1H), 7.49-7.16 (m, 7H), 5.37 (s, 2H), 4.54-3.77 (m, 10H), 1.46-0.92 (m, 9H), 0.81- 0.67 (m, 1H). | m/z = 552.4 [M + H]⁺ |
| I-12 | 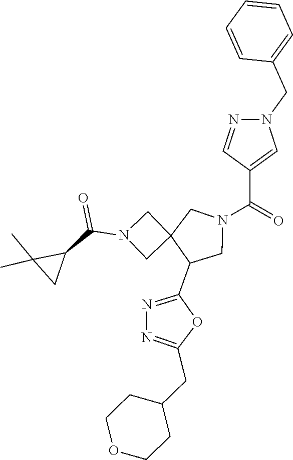 [SMILES: CC1(C)C[C@@H]1C(=O)N1CC2(CN(C(=O)c3cnn(Cc4ccccc4)c3)CC2c2nnc(CC3CCOCC3)o2)C1] | ¹H NMR (400 MHz, CD₃OD) δ 8.35-8.19 (m, 1H), 7.98-7.87 (m, 1H), 7.45-7.19 (m, 5H), 5.38 (s, 2H), 4.56-4.31 (m, 1H), 4.29-4.08 (m, 4H), 4.08-3.96 (m, 3H), 3.94-3.86 (m, 3H), 3.38 (t, J = 11.8 Hz, 2H), 2.92- 2.77 (m, 2H), 2.13- 1.98 (m, 1H), 1.70-1.54 (m, 2H), 1.49-1.32 (m, 3H), 1.24-1.15 (m, 2H), 1.14-1.11 (m, 2H), 1.10- 1.08 (m, 1H), 1.06- 0.98 (m, 2H), 0.81-0.71 (m, 1H). | m/z = 558.3 [M + H]⁺ |
| I-13 | 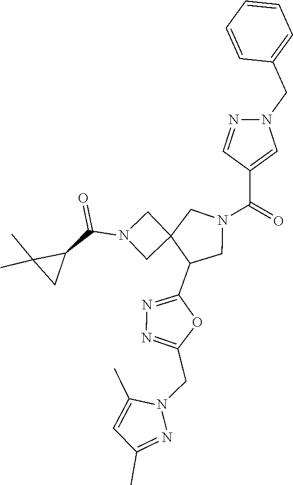 [SMILES: Cc1cc(C)n(Cc2nnc(C3CN(C(=O)c4cnn(Cc5ccccc5)c4)CC34CN(C(=O)[C@H]3CC3(C)C)C4)o2)n1] | ¹H NMR (400 MHz, Methanol-d4) δ 8.28- 8.15 (m, 1H), 7.97-7.85 (m, 1H), 7.40-7.23 (m, 5H), 5.87 (d, J = 6.6 Hz, 1H), 5.50-5.43 (m, 2H), 5.38 (d, J = 2.0 Hz, 2H), 4.44-3.86 (m, 9H), 2.29 (d, J = 3.2 Hz, 3H), 2.12 (s, 3H), 1.46-1.30 (m, 1H), 1.22-1.00 (m, 7H), 0.78-0.72 (m, 1H). | m/z = 569.4 [M + H]⁺ |
| I-4 | 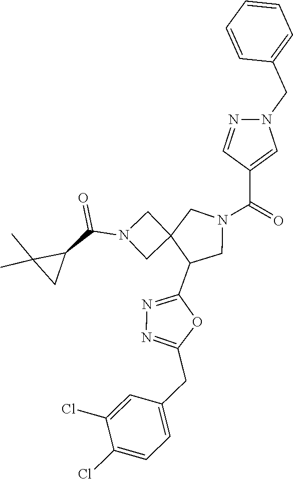 [SMILES: CC1(C)C[C@@H]1C(=O)N1CC2(CN(C(=O)c3cnn(Cc4ccccc4)c3)CC2c2nnc(Cc3ccc(Cl)c(Cl)c3)o2)C1] | ¹H NMR (400 MHz, CD₃OD) δ 8.25-8.17 (m, 1H), 7.95-7.87 (m, 1H), 7.56-7.50 (m, 1H), 7.49- 7.44 (m, 1H), 7.40-7.14 (m, 6H), 5.38 (s, 2H), 4.45-3.85 (m, 11H), 1.39-1.31 (m, 1H), 1.26- 0.95 (m, 7H), 0.80-0.70 (m, 1H). | m/z = 619.3 [M + H]⁺ |
| I-20 | 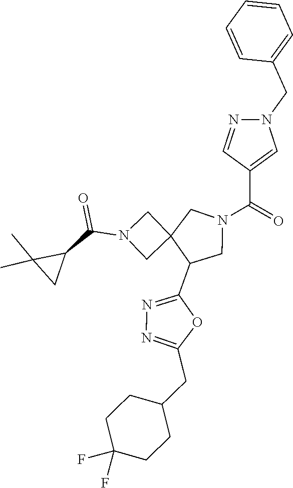 [SMILES: CC1(C)C[C@@H]1C(=O)N1CC2(CN(C(=O)c3cnn(Cc4ccccc4)c3)CC2c2nnc(CC3CCC(F)(F)CC3)o2)C1] | ¹H NMR (400 MHz, Methanol-d4) δ 8.24 (d, J = 8.1 Hz, 1H), 7.94 (d, J = 6.9 Hz, 1H), 7.32 (d, J = 15.6 Hz, 4H), 5.39 (s, 2H), 4.59-3.88 (m, 9H), 2.86 (d, J = 6.3 Hz, 2H), 2.09-1.66 (m, 7H), 1.35 (d, J = 7.2 Hz, 4H), 1.24- 1.01 (m, 7H), 0.84- 0.68 (m, 1H). | m/z = 593.5 [M + H]⁺ |
| I-22 | 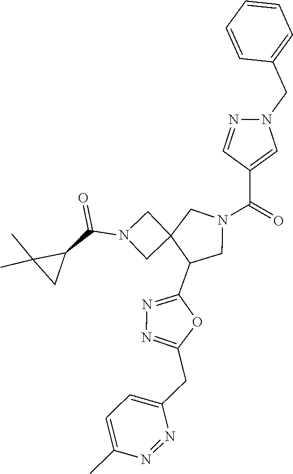 [SMILES: Cc1ccc(Cc2nnc(C3CN(C(=O)c4cnn(Cc5ccccc5)c4)CC34CN(C(=O)[C@H]3CC3(C)C)C4)o2)nn1] | ¹H NMR (400 MHz, CD₃OD) δ 8.26-8.19 (m, 1H), 7.92 (d, J = 5.1 Hz, 1H), 7.73-7.52 (m, 2H), 7.31 (ddt, J = 21.2, 9.1, 4.5 Hz, 5H), 5.37 (s, 2H), 4.60 (q, J = 4.2, 3.4 Hz, 2H), 4.47-3.87 (m, 9H), 2.65 (s, 3H), 1.42- 1.33 (m, 1H), 1.19 - 0.99 (m, 7H), 0.83-0.70 (m, 1H). | m/z = 567.4 [M + H]⁺ |
| I-17 | 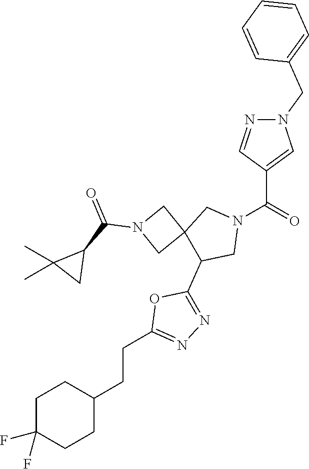 [SMILES: CC1(C)C[C@@H]1C(=O)N1CC2(CN(C(=O)c3cnn(Cc4ccccc4)c3)CC2c2nnc(CCC3CCC(F)(F)CC3)o2)C1] | ¹H NMR (400 MHz, CD₃OD) δ 8.23 (d, J = 10.5 Hz, 1H), 7.94 (d, J = 7.3 Hz, 1H), 7.41-7.21 (m, 5H), 5.38 (s, 2H), 4.54-3.92 (m, 9H), 2.92 (dd, J = 9.4, 5.8 Hz, 2H), 2.01 (h, J = 7.3 Hz, 2H), 1.86-1.67 (m, 6H), 1.51- 1.09 (m, 10H), 1.02 (s, 1H), 0.79-0.76 (m, 1H). | m/z = 607.4 [M + H]⁺ |
| I-18 | 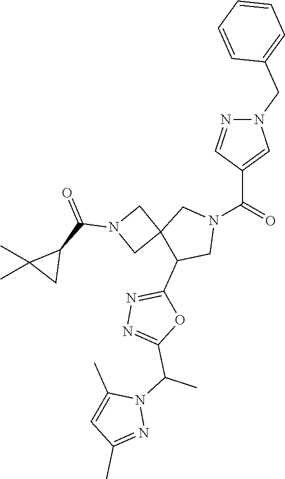 [SMILES: Cc1cc(C)n(C(C)c2nnc(C3CN(C(=O)c4cnn(Cc5ccccc5)c4)CC34CN(C(=O)[C@H]3CC3(C)C)C4)o2)n1] | ¹H NMR (400 MHz, CD₃OD) ¹H NMR (400 MHz, Methanol-d4) δ 826-8.15 (m, 1H), 7.99- 7.86 (m, 1H), 7.41- 7.19 (m, 5H), 5.88-5.78 (m, 1H), 5.39-5.34 (m, 2H), 4.40-3.87 (m, 9H), 2.40-2.20 (m, 3H), 2.15- 2.04 (m, 3H), 1.91 (dt, J = 5.2, 2.8 Hz, 3H), 1.41- 1.27 (m, 2H), 1.17- 1.03 (m, 6H), 0.77 (dd, J = 8.1, 4.3 Hz, 1H). | m/z = 583.4 (M + H]⁺ |

### Synthesis of (1-benzyl-1H-pyrazol-4-yl)((R)-8-(5-(3,4-dichlorobenzyl)-1,3,4-oxadiazol-2-yl)-2-((S)-2,2-dimethylcyclopropane-1-carbonyl)-2,6-diazaspiro[3.4]octan-6-yl)methanone (I-16)

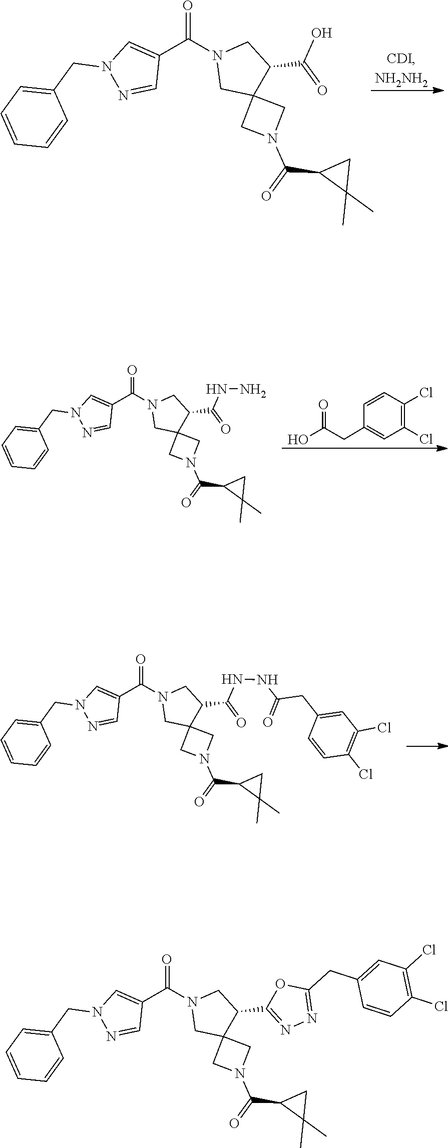

(1-benzyl-1H-pyrazol-4-yl)((R)-8-(5-(3,4-dichlorobenzyl)-1,3,4-oxadiazol-2-yl)-2-((S)-2,2-dimethylcyclopropane-1-carbonyl)-2,6-diazaspiro[3.4]octan-6-yl)methanone was synthesized from (R)-6-(1-benzyl-1H-pyrazole-4-carbonyl)-2-((S)-2,2-dimethylcyclopropane-1-carbonyl)-2,6-diazaspiro[3.4]octane-8-carboxylic acid according to the procedures outlined for I-14 using the appropriate commercially available reagents and/or intermediates described elsewhere. LCMS m/z=619.3 [M+H]⁻; ¹H NMR (400 MHz, CD₃OD) δ 8.24-8.17 (m, 1H), 7.94-7.87 (m, 1H), 7.54-7.42 (m, 2H), 7.36-7.18 (m, 6H), 5.36 (s, 2H), 4.36-3.83 (m, 11H), 1.40-1.20 (m, 2H), 1.18-0.97 (m, 6H), 0.79-0.69 (m, 1H).

### Synthesis of (1-benzyl-1H-pyrazol-4-yl)((S)-8-(5-(3,4-dichlorobenzyl)-1,3,4-oxadiazol-2-yl)-2-((S)-2,2-dimethylcyclopropane-1-carbonyl)-2,6-diazaspiro[3.4]octan-6-yl)methanone (I-15)

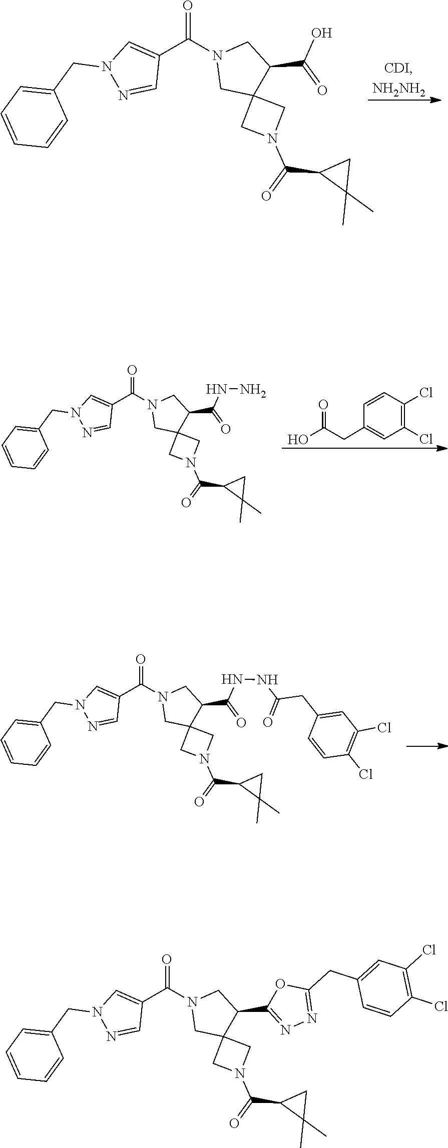

(1-benzyl-1H-pyrazol-4-yl)((S)-8-(5-(3,4-dichlorobenzyl)-1,3,4-oxadiazol-2-yl)-2-((S)-2,2-dimethylcyclopropane-1-carbonyl)-2,6-diazaspiro[3.4]octan-6-yl)methanone was synthesized from (S)-6-(1-benzyl-1H-pyrazole-4-carbonyl)-2-((S)-2,2-dimethylcyclopropane-1-carbonyl)-2,6-diazaspiro[3.4]octane-8-carboxylic acid according to the procedures outlined for I-14 using the appropriate commercially available reagents and/or intermediates described elsewhere. LCMS: m/z=619.2 [M+H]⁺; ¹H NMR (400 MHz, CD₃OD) δ 8.22 (d, J=11.3 Hz, 1H), 7.93 (d, J=8.6 Hz, 1H), 7.55 (d, J=2.3 Hz, 1H), 7.48 (dd, J=8.2, 2.8 Hz, 1H), 7.39-7.14 (m, 6H), 5.38 (s, 2H), 4.27-3.88 (m, 10H), 1.39-1.29 (m, 2H), 1.19-0.97 (m, 7H), 0.83-0.70 (m, 1H).

### Synthesis of (1-benzyl-1H-pyrazol-4-yl)(8-(5-(3,4-dichlorobenzyl)-4H-1,2,4-triazol-3-yl)-2-((S)-2,2-dimethylcyclopropane-1-carbonyl)-2,6-diazaspiro[3.4]octan-6-yl)methanone (I-6)

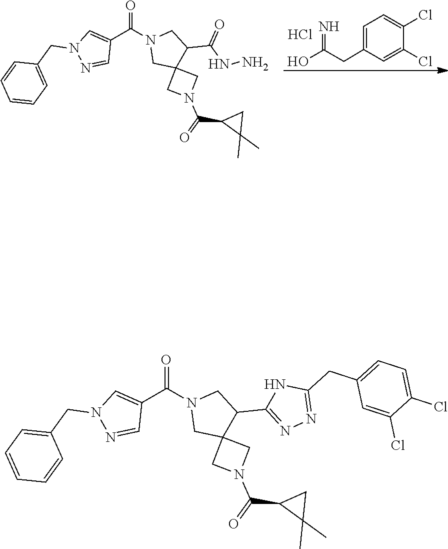 [SMILES: CC1(C)C[C@@H]1C(=O)N1CC2(CN(C(=O)c3cnn(Cc4ccccc4)c3)CC2C(=O)NN)C1 ; Cl ; N=C(O)Cc1ccc(Cl)c(Cl)c1 ; CC1(C)C[C@@H]1C(=O)N1CC2(CN(C(=O)c3cnn(Cc4ccccc4)c3)CC2c2nnc(Cc3ccc(Cl)c(Cl)c3)[nH]2)C1]

### Step 1: (1-benzyl-1H-pyrazol-4-yl)(8-(5-(3,4-dichlorobenzyl)-4H-1,2,4-triazol-3-yl)-2-((S)-2,2-dimethylcyclopropane-1-carbonyl)-2,6-diazaspiro[3.4]octan-6-yl)methanone

To a solution of ethyl 2-(3,4-dichlorophenyl)acetimidate hydrochloride (29.4 mg, 0.089 mmol) and 6-(1-benzyl-1H-pyrazole-4-carbonyl)-2-((S)-2,2-dimethylcyclopropane-1-carbonyl)-2,6-diazaspiro[3.4]octane-8-carbohydrazide (40 mg, 0.089 mmol) in EtOH (3.0 mL) was added NH₄OAc (20.5 mg, 0.27 mmol). The reaction was heated at reflux for 14 h then diluted with water and extracted with EtOAc (30 mL×2). The combined organic layers were washed with water, brine and dried over Na₂SO₄ and concentrated. The residue was purified by prep-HPLC to give (1-benzyl-1H-pyrazol-4-yl)(8-(5-(3,4-dichlorobenzyl)-4H-1,2,4-triazol-3-yl)-2-((S)-2,2-dimethylcyclopropane-1-carbonyl)-2,6-diazaspiro[3.4]octan-6-yl)methanone (12 mg, 21%) as a colorless oil. LCMS m/z=618.3 [M+H]⁺; ¹H NMR (400 MHz, CD₃OD) δ 8.23 (d, J=9.6 Hz, 1H), 7.94 (d, J=6.2 Hz, 1H), 7.54-7.07 (m, 8H), 5.38 (d, J=5.6 Hz, 2H), 4.42-3.66 (m, 11H), 1.42-1.33 (m, 1H), 1.20-0.92 (m, 7H), 0.77-0.63 (m, 1H).

### Synthesis of (2-(oxazol-2-yl)-8-(5-((6-(tetrahydro-2H-pyran-4-yl)pyridin-2-yl)methyl)-1,3,4-oxadiazol-2-yl)-2,6-diazaspiro[3.4]octan-6-yl)(thiazol-5-yl)methanone (I-80)

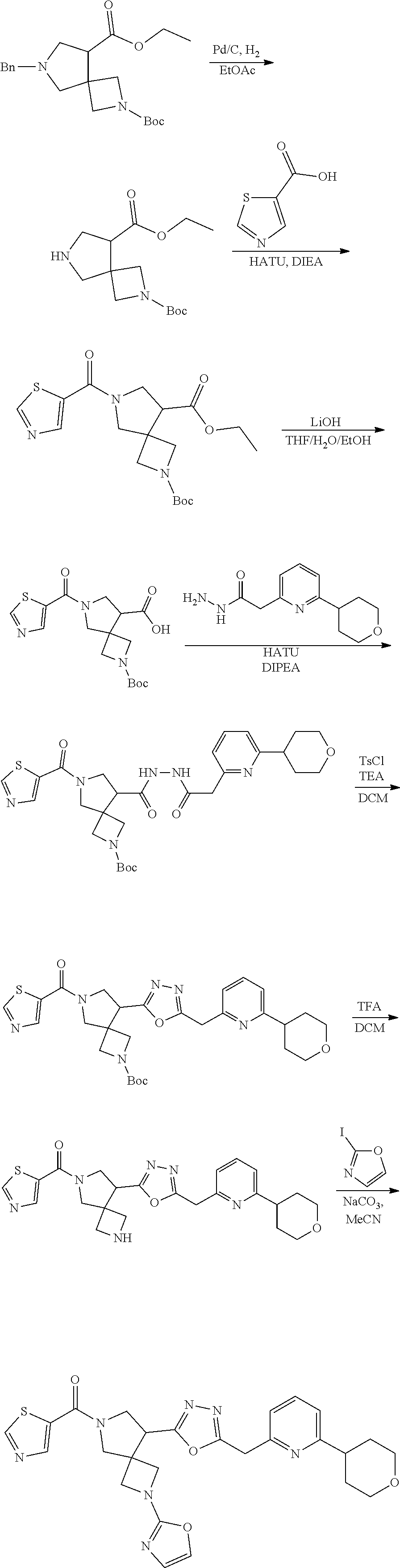

### Step 1: 2-(tert-butyl) 8-ethyl 2,6-diazaspiro[3.4]octane-2,8-dicarboxylate

To a solution of 2-(tert-butyl) 8-ethyl 6-benzyl-2,6-diazaspiro[3.4]octane-2,8-dicarboxylate (2.0 g, 5.3 mmol) in EtOAc (8 mL) was added 10% Pd/C (600 mg). The reaction mixture was stirred under a H₂ atmosphere for 24 h then the catalyst was removed by filtration through celite. The filtrate was concentrated to afford 2-(tert-butyl) 8-ethyl 2,6-diazaspiro[3.4]octane-2,8-dicarboxylate (1.5 g, 99%) which was used directly in the next step. LCMS n z=285.1 [M+H]⁺.

### Step 2: 2-(tert-butyl) 8-ethyl 6-(thiazole-5-carbonyl)-2,6-diazaspiro[3.4]octane-2,8-dicarboxylate

To a solution of thiazole-5-carboxylic acid (680 mg, 5.28 mmol) in DCM (20 mL) was added HATU (2.0 g, 5.28 mmol) and DIPEA (1.7 g, 13.2 mmol). The mixture was stirred at room temperature for 30 min then 2-(tert-butyl) 8-ethyl 2,6-diazaspiro[3.4]octane-2,8-dicarboxylate (1.5 g, 5.28 mmol) was added. The reaction was stirred at room temperature for another 3 h then diluted with water (30 mL) and extracted with DCM (100 mL×2). The combined organic layers were washed with brine, dried over Na₂SO₄, filtered and concentrated. The mixture was purified by column chromatography on silica gel (eluent: DCM:MeOH=50:1) to afford 2-(tert-butyl) 8-ethyl 6-(thiazole-5-carbonyl)-2,6-diazaspiro[3.4]octane-2,8-dicarboxylate (1.7 g, 85% yield) as a yellow solid. LCMS m/z=296 [M+H−100]⁺; ¹H NMR (400 MHz, CDCl₃) δ 8.86 (s, 1H), 8.16 (s, 1H), 4.22-4.10 (m, 2H), 3.96-3.92 (m, 2H), 3.80-3.75 (m, 2H), 3.66-3.60 (m, 2H), 3.15-3.08 (m, 2H), 2.89-2.84 (m, 1H), 1.31 (s, 9H), 1.23-1.19 (m, 3H).

### Step 3: 2-(tert-butoxycarbonyl)-6-(thiazole-5-carbonyl)-2,6-diazaspiro[3.4]octane-8-carboxylic acid

To a solution of 2-(tert-butyl) 8-ethyl 6-(thiazole-5-carbonyl)-2,6-diazaspiro[3.4]octane-2,8-dicarboxylate (1.7 g, 4.3 mmol) in a mixture of THF, EtOH and water (16 mL/4 mL/4 mL) at 0° C. was added lithium hydroxide monohydrate (206 mg, 8.6 mmol). The reaction mixture was stirred at 0° C. for 1 h, diluted with water (40 mL) and extracted with EtOAc (70 mL). The aqueous layer was collected, acidified to pH˜2 with 1M HCl and extracted with EtOAc (70 mL×3). The combined organic layers were washed with brine, dried over Na₂SO₄, filtered and concentrated to afford crude 2-(tert-butoxycarbonyl)-6-(thiazole-5-carbonyl)-2,6-diazaspiro[3.4]octane-8-carboxylic acid (1.3 g, 82%) as a yellow solid which was used directly in the next step. LCMS m/z=312.00 [M+H−56]⁺.

### Step 4: tert-butyl 8-(2-(2-(6-(tetrahydro-2H-pyran-4-yl)pyridin-2-yl)acetyl)hydrazine-1-carbonyl)-6-(thiazole-5-carbonyl)-2,6-diazaspiro[3.4]octane-2-carboxylate

To a solution of 2-(tert-butoxycarbonyl)-6-(thiazole-5-carbonyl)-2,6-diazaspiro[3.4]octane-8-carboxylic acid (474 mg, 1.29 mmol) in DCM (6 mL) was added HATU (426 mg, 1.12 mmol) and DIPEA (361 g, 2.8 mmol). The mixture was stirred at room temperature for 30 min then 2-(6-(tetrahydro-2H-pyran-4-yl)pyridin-2-yl)acetohydrazide (264 mg, 1.12 mmol) was added. The reaction was stirred at room temperature for another 3 h then diluted with water (20 mL) and extracted with DCM (40 mL×2). The combined organic layers were washed with brine, dried over Na₂SO₄, filtered and concentrated. The mixture was purified by prep-TLC (eluent: DCM:MeOH=20:1) to afford tert-butyl 8-(2-(2-(6-(tetrahydro-2H-pyran-4-yl)pyridin-2-yl)acetyl)hydrazine-1-carbonyl)-6-(thiazole-5-carbonyl)-2,6-diazaspiro[3.4]octane-2-carboxylate (250 mg, 38% yield) as a colorless oil. LCMS m/z=585.15 [M+H]⁺.

### Step 5: tert-butyl 8-(5-((6-(tetrahydro-2H-pyran-4-yl)pyridin-2-yl)methyl)-1,3,4-oxadiazol-2-yl)-6-(thiazole-5-carbonyl)-2,6-diazaspiro[3.4]octane-2-carboxylate

To a solution of tert-butyl 8-(2-(2-(6-(tetrahydro-2H-pyran-4-yl)pyridin-2-yl)acetyl)hydrazine-1-carbonyl)-6-(thiazole-5-carbonyl)-2,6-diazaspiro[3.4]octane-2-carboxylate (250 mg, 0.43 mmol) in DCM (4 mL) was added TEA (85 mg, 0.85 mmol) and TsCl (163 mg, 0.85 mmol). The reaction mixture was stirred at room temperature overnight then concentrated under reduced pressure. The residue obtained was purified by prep-TLC (eluent: DCM:MeOH=20:1) to afford tert-butyl 8-(5-((6-(tetrahydro-2H-pyran-4-yl)pyridin-2-yl)methyl)-1,3,4-oxadiazol-2-yl)-6-(thiazole-5-carbonyl)-2,6-diazaspiro[3.4]octane-2-carboxylate (100 mg, 42% yield) as a yellow oil. LCMS m/z=567.20 [M+H]⁺; ¹H NMR (400 MHz, CDCl₃) δ 8.92 (s, 1H), 8.24 (s, 11H), 7.62 (t, J=7.8 Hz, 1H), 7.09 (d, J=7.8 Hz, 2H), 4.39 (s, 2H), 4.28-4.20 (m, 1H), 4.12-4.02 (m, 6H), 3.93-3.86 (m, 2H), 3.81-3.74 (m, 1H), 3.58-3.48 (m, 3H), 2.96-2.87 (m, 1H), 1.85-1.81 (m, 4H), 1.42 (s, 9H).

### Step 6: (8-(5-((6-(tetrahydro-2H-pyran-4-yl)pyridin-2-yl)methyl)-1,3,4-oxadiazol-2-yl)-2,6-diazaspiro[3.4]octan-6-yl)(thiazol-5-yl)methanone

To a solution of tert-butyl 8-(5-((6-(tetrahydro-2H-pyran-4-yl)pyridin-2-yl)methyl)-1,3,4-oxadiazol-2-yl)-6-(thiazole-5-carbonyl)-2,6-diazaspiro[3.4]octane-2-carboxylate (100 mg, 0.18 mmol) in DCM (2 mL) was added TFA (1 mL) and the reaction stirred at room temperature for 2 h. The solvent was removed under reduced pressure and exchanged by SCX-column to afford (8-(5-((6-(tetrahydro-2H-pyran-4-yl)pyridin-2-yl)methyl)-1,3,4-oxadiazol-2-yl)-2,6-diazaspiro[3.4]octan-6-yl)(thiazol-5-yl)methanone (60 mg, 73% yield) as a yellow solid. LCMS m/z=467.15 [M+H]⁺.

### Step 7: (2-(oxazol-2-yl)-8-(5-((6-(tetrahydro-2H-pyran-4-yl)pyridin-2-yl)methyl)-1,3,4-oxadiazol-2-yl)-2,6-diazaspiro[3.4]octan-6-yl)(thiazol-5-yl)methanone

To a solution of (8-(5-((6-(tetrahydro-2H-pyran-4-yl)pyridin-2-yl)methyl)-1,3,4-oxadiazol-2-yl)-2,6-diazaspiro[3.4]octan-6-yl)(thiazol-5-yl)methanone (50 mg, 0.11 mmol) in MeCN was added Na₂CO₃ (35 mg, 0.33 mmol) and 2-iodooxazole (43 mg, 0.22 mmol). The reaction mixture was heated at reflux for 8 h then the solids removed by filtration. The filtrate was concentrated and the residue obtained purified by prep-TLC (eluent: DCM:MeOH=10:1) to afford (2-(oxazol-2-yl)-8-(5-((6-(tetrahydro-2H-pyran-4-yl)pyridin-2-yl)methyl)-1,3,4-oxadiazol-2-yl)-2,6-diazaspiro[3.4]octan-6-yl)(thiazol-5-yl)methanone (20 mg, 33%) as a white solid. LCMS m/z=534.2 [M+H]⁺; ¹H NMR (400 MHz, DMSO-d₆) δ 9.26 (s, 1H), 8.40-8.34 (m, 1H), 7.66 (t, J=7.8 Hz, 11H), 7.59-7.56 (m, 1H), 7.20-7.14 (m, 2H), 6.85-6.81 (m, 11H), 4.42-4.37 (m, 2H), 4.28-4.21 (m, 1H), 4.19-4.08 (m, 4H), 4.03-3.98 (m, 1H), 3.96-3.82 (m, 5H), 3.45-3.35 (m, 2H), 2.89-2.79 (m, 1H), 1.71-1.62 (m, 4H).

### Synthesis of (8-(5-((3,4-dichlorophenyl)difluoromethyl)-1,3,4-oxadiazol-2-yl)-2-((S)-2,2-dimethylcyclopropane-1-carbonyl)-8-fluoro-2,6-diazaspiro[3.4]octan-6-yl)(thiazol-5-yl)methanone, I-38 diastereomeric mixture

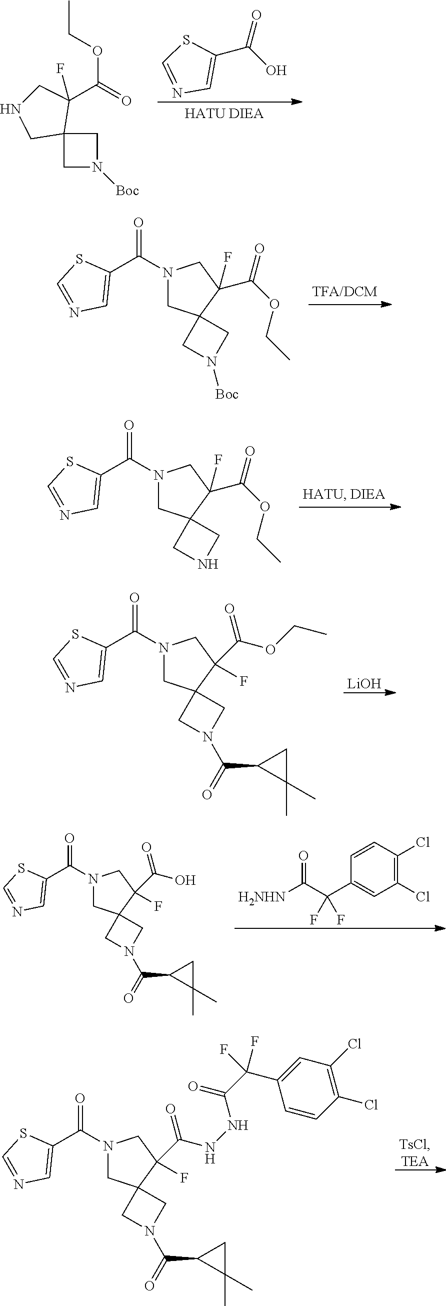

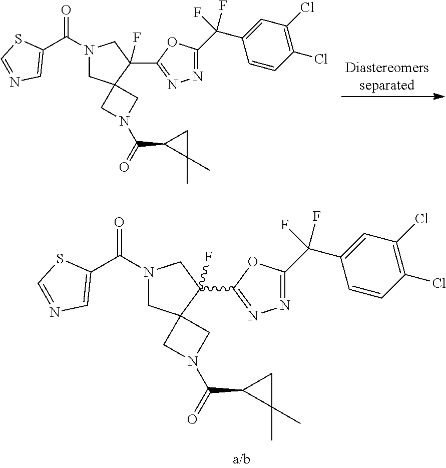

### Step 1: 2-(tert-butyl) 8-ethyl 8-fluoro-6-(thiazole-5-carbonyl)-2,6-diazaspiro[3.4]octane-2,8-dicarboxylate

To a solution of thiazole-5-carboxylic acid (427 mg, 3.31 mmol) in DCM (15 mL) was added HATU (1.51 g, 3.97 mmol) and DIPEA (854 mg, 6.62 mmol). The mixture was stirred at room temperature for 10 min then 2-(tert-butyl) 8-ethyl 8-fluoro-2,6-diazaspiro[3.4]octane-2,8-dicarboxylate (1000 mg, 3.31 mmol) was added and the reaction was stirred for another 2 h. The reaction was diluted with water (30 mL) and extracted with DCM (30 mL×2). The combined organic layers were washed with brine, dried over Na₂SO₄, filtered and concentrated. The residue obtained was purified by silica gel column (eluent: Pet.Ether:EtOAc=2:1) to afford 2-(tert-butyl) 8-ethyl 8-fluoro-6-(thiazole-5-carbonyl)-2,6-diazaspiro[3.4]octane-2,8-dicarboxylate (2 g, contain DIPEA) as a colorless oil. ¹H NMR (400 MHz, DMSO-d6) δ 9.28 (d, J=5.6 Hz, 1H), 8.40 (d, J=26.6 Hz, 1H), 4.31 (m, 2H), 4.17-4.01 (m, 4H), 3.83-3.73 (m, 1H), 3.62 (dt, J=6.6, 3.2 Hz, 1H), 3.15-3.11 (m, 1H), 1.39-1.34 (m, 9H), 1.23 (s, 3H).

### Step 2: ethyl 8-fluoro-6-(thiazole-5-carbonyl)-2,6-diazaspiro[3.4]octane-8-carboxylate

To a solution of 2-(tert-butyl) 8-ethyl 8-fluoro-6-(thiazole-5-carbonyl)-2,6-diazaspiro[3.4]octane-2,8-dicarboxylate (2 g, 4.84 mmol) in DCM (12 mL) was added TFA (3 mL). The reaction mixture was stirred at room temperature for 3 h then the solvent was removed under reduced pressure to afford crude ethyl 8-fluoro-6-(thiazole-5-carbonyl)-2,6-diazaspiro[3.4]octane-8-carboxylate (1.4 g, 92%, colorless oil) which was used directly in the next step.

### Step 3: ethyl 2-((S)-2,2-dimethylcyclopropane-1-carbonyl)-8-fluoro-6-(thiazole-5-carbonyl)-2,6-diazaspiro[3.4]octane-8-carboxylate

To a solution of (S)-2,2-dimethylcyclopropane-1-carboxylic acid (431 mg, 3.77 mmol) in DCM (5 mL) was added HATU (2153 mg, 5.66 mmol) and DIPEA (1464 mg, 11.32 mmol). The mixture was stirred at room temperature for 10 min then ethyl 8-fluoro-6-(thiazole-5-carbonyl)-2,6-diazaspiro[3.4]octane-8-carboxylate (1300 mg, 4.15 mmol) was added and the reaction stirred for another 2 h. The mixture was diluted with water (20 mL) and extracted with DCM (30 mL×2). The combined organic layers were washed with brine, dried over Na₂SO₄, filtered and concentrated. The residue obtained was purified by silica gel column (eluent: DCM:MeOH=100:1) to afford 2-((S)-2,2-dimethylcyclopropane-1-carbonyl)-8-fluoro-6-(thiazole-5-carbonyl)-2,6-diazaspiro[3.4]octane-8-carboxylate (800 mg, 47%) as a colorless oil. LCMS m/z=382.05 [M+H]⁺. ¹H NMR (400 MHz, DMSO-d6) δ 9.29 (s, 1H), 8.42 (m, 1H), 4.46-3.80 (m, 10H), 1.26 (s, 4H), 1.15-1.01 (m, 6H), 0.86 (s, 1H), 0.71 (s, 1H).

### Step 4: 2-((S)-2,2-dimethylcyclopropane-1-carbonyl)-8-fluoro-6-(thiazole-5-carbonyl)-2,6-diazaspiro[3.4]octane-8-carboxylic acid

To a solution of ethyl 2-((S)-2,2-dimethylcyclopropane-1-carbonyl)-8-fluoro-6-(thiazole-5-carbonyl)-2,6-diazaspiro[3.4]octane-8-carboxylate (400 mg, 0.98 mmol) in a mixture of THF, MeOH and H₂O (6 mL/2 mL/2 mL) was added LiOH (103 mg, 2.44 mmol). The reaction stirred at room temperature for 2 h, then diluted with water (15 mL) and extracted with EtOAc (10 mL×2). The aqueous layer was collected, acidified to pH˜2 with 1M HCl and extracted with EtOAc (100 mL×2). The combined organic layers was washed with brine, dried over Na₂SO₄, filtered and concentrated to afford 2-((S)-2,2-dimethylcyclopropane-1-carbonyl)-8-fluoro-6-(thiazole-5-carbonyl)-2,6-diazaspiro[3.4]octane-8-carboxylic acid (300 mg, 80%) as a white solid which was used directly in the next step. LCMS m/z=382.05 [M+H]⁺; ¹H NMR (400 MHz, DMSO-d6) δ 9.28 (d, J=4.8 Hz, 1H), 8.47-8.37 (m, 1H), 4.43-3.78 (m, 8H), 1.14-1.02 (m, 6H), 0.86 (d, J=5.8 Hz, 2H), 0.69 (s, 1H).

### Step 5: N′-(2-(3,4-dichlorophenyl)-2,2-difluoroacetyl)-2-((S)-2,2-dimethylcyclopropane-1-carbonyl)-8-fluoro-6-(thiazole-5-carbonyl)-2,6-diazaspiro[3.4]octane-8-carbohydrazide

To a solution of 2-((S)-2,2-dimethylcyclopropane-1-carbonyl)-8-fluoro-6-(thiazole-5-carbonyl)-2,6-diazaspiro[3.4]octane-8-carboxylic acid (205 mg, 0.54 mmol) in DCM (4 mL) was added EDCI (124 mg, 0.65 mmol), HOBt (87.2 mg, 0.65 mmol) and DIPEA (209 mg, 1.61 mmol). The reaction was stirred at room temperature for 5 min then 2-(3,4-dichlorophenyl)-2,2-difluoroacetohydrazide (164 mg, 0.65 mmol) was added and the reaction stirred for another 2 h. The reaction was diluted with water (20 mL) and extracted with DCM (50 mL×2). The combined organic layers were washed with brine, dried over Na₂SO₄, filtered and concentrated. The residue obtained was purified by prep-TLC (eluent: DCM:MeOH=20:1) to afford N′-(2-(3,4-dichlorophenyl)-2,2-difluoroacetyl)-2-((S)-2,2-dimethylcyclopropane-1-carbonyl)-8-fluoro-6-(thiazole-5-carbonyl)-2,6-diazaspiro[3.4]octane-8-carbohydrazide (105 mg, 32%) as a white solid. LCMS m/z=618.0 [M+H]⁺.

### Step 6: (8-(5-((3,4-dichlorophenyl)difluoromethyl)-1,3,4-oxadiazol-2-yl)-2-((S)-2,2-dimethylcyclopropane-1-carbonyl)-8-fluoro-2,6-diazaspiro[3.4]octan-6-yl)(thiazol-5-yl)methanone

To a solution of N′-(2-(3,4-dichlorophenyl)-2,2-difluoroacetyl)-2-((S)-2,2-dimethylcyclopropane-1-carbonyl)-8-fluoro-6-(thiazole-5-carbonyl)-2,6-diazaspiro[3.4]octane-8-carbohydrazide (40 mg, 0.06 mmol) in DCM (2 mL) was added TEA (33 mg, 0.32 mmol) and TsCl (38 mg, 0.19 mmol). The reaction mixture was stirred at room temperature overnight then concentrated under reduced pressure and purified by prep-TLC (eluent: Pet.Ether:EtOAc=1:1) to afford (8-(5-((3,4-dichlorophenyl)difluoromethyl)-1,3,4-oxadiazol-2-yl)-2-((S)-2,2-dimethylcyclopropane-1-carbonyl)-8-fluoro-2,6-diazaspiro[3.4]octan-6-yl)(thiazol-5-yl)methanone (15 mg, 39% yield) as a white solid. LCMS m/z=600.1 [M+H]⁺; ¹H NMR (400 MHz, DMSO-d₆) δ 9.31 (d, J=6.4 Hz, 1H), 8.46 (m, 1H), 8.06 (s, 1H), 7.94-7.87 (m, 1H), 7.75 (s, 1H), 4.59-3.91 (m, 8H), 1.12 (d, J=5.2 Hz, 2H), 1.05-1.00 (m, 3H), 0.93 (d, J=13.6 Hz, 1H), 0.85 (d, J=6.0 Hz, 2H), 0.69 (s, 1H). 85 mg of the racemic material was separated by chiral HPLC to afford the first eluting fraction (8.4 mg I-38) LCMS m/z=600.1 [M+H]⁺; ¹H NMR (400 MHz, Methanol-d₄) δ 9.20 (s, 1H), 8.43 (d, J=18.8 Hz, 1H), 7.93 (s, 1H), 7.76 (t, J=7.4 Hz, 1H), 7.66 (d, J=9.2 Hz, 1H), 4.62-4.39 (m, 5H), 4.19 (m, 2H), 3.97 (s, 1H), 1.37 (m, 1H), 1.07 (m, 7H), 0.76 (d, J=34.4 Hz, 1H). And the second eluting fraction (6.4 mg I-38) LCMS m/z=600.1 [M+H]⁺; ¹H NMR (400 MHz, Methanol-d₄) δ 9.23 (s, 1H), 8.46 (d, J=16.4 Hz, 1H), 7.96 (d, J=6.8 Hz, 1H), 7.80 (t, J=7.6 Hz, 1H), 7.68 (t, J=10.0 Hz, 1H), 4.78 (d, J=9.6 Hz, 1H), 4.52 (m, 5H), 4.25-3.78 (m, 2H), 1.42-1.31 (m, 1H), 1.13 (m, 7H), 0.80 (d, J=28.0 Hz, 1H).

### Synthesis of ((S)-2,2-dimethylcyclopropyl)(8-(5-((6-(tetrahydro-2H-pyran-4-yl)pyridin-2-yl)methyl)-1,3,4-oxadiazol-2-yl)-6-(thiazolo[4,5-d]pyrimidin-7-yl)-2,6-diazaspiro[3.4]octan-2-yl)methanone (I-105)

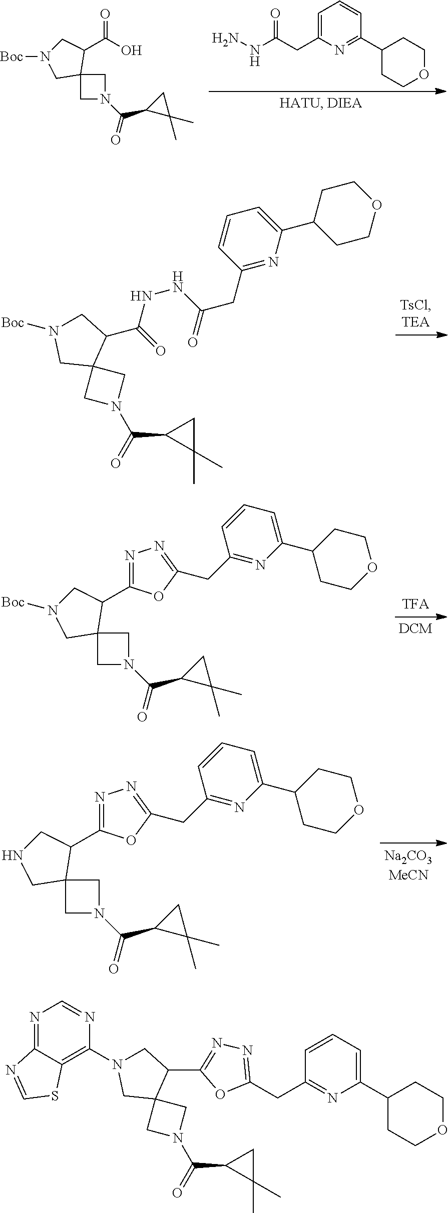

### Step 1: tert-butyl 2-((S)-2,2-dimethylcyclopropane-1-carbonyl)-8-(2-(2-(6-(tetrahydro-2H-pyran-4-yl)pyridin-2-yl)acetyl)hydrazine-1-carbonyl)-2,6-diazaspiro[3.4]octane-6-carboxylate

To a solution of 6-(tert-butoxycarbonyl)-2-((S)-2,2-dimethylcyclopropane-1-carbonyl)-2,6-diazaspiro[3.4]octane-8-carboxylic acid (300 mg, 0.85 mmol) in DCM (5 mL) was added HATU (270 mg, 0.71 mmol) and DIPEA (229 mg, 1.78 mmol). The reaction was stirred at room temperature for 30 min then 2-(6-(tetrahydro-2H-pyran-4-yl)pyridin-2-yl)acetohydrazide (167 mg, 0.71 mmol) was added and the reaction was stirred for another 2 h. The mixture was diluted with water (20 mL) and extracted with DCM (30 mL×2). The combined organic layers were washed with brine, dried over Na₂SO₄, filtered and concentrated. The residue obtained was purified by prep-TLC (eluent: DCM:MeOH=20:1) to afford tert-butyl 2-((S)-2,2-dimethylcyclopropane-1-carbonyl)-8-(2-(2-(6-(tetrahydro-2H-pyran-4-yl)pyridin-2-yl)acetyl)hydrazine-1-carbonyl)-2,6-diazaspiro[3.4]octane-6-carboxylate (350 mg, 72%) as a white solid. LCMS m/z=570.2 [M+H]⁺.

### Step 2: tert-butyl 2-((S)-2,2-dimethylcyclopropane-1-carbonyl)-8-(5-((6-(tetrahydro-2H-pyran-4-yl)pyridin-2-yl)methyl)-1,3,4-oxadiazol-2-yl)-2,6-diazaspiro[3.4]octane-6-carboxylate

To a solution of tert-butyl 2-((S)-2,2-dimethylcyclopropane-1-carbonyl)-8-(2-(2-(6-(tetrahydro-2H-pyran-4-yl)pyridin-2-yl)acetyl)hydrazine-1-carbonyl)-2,6-diazaspiro[3.4]octane-6-carboxylate (350 mg, 0.43 mmol) in DCM (4 mL) was added TEA (186 mg, 1.85 mmol) and TsCl (351 mg, 1.85 mmol). The reaction mixture was stirred at room temperature overnight then was concentrated under reduced pressure and purified by prep-TLC (eluent: DCM:MeOH=20:1) to afford tert-butyl 2-((S)-2,2-dimethylcyclopropane-1-carbonyl)-8-(5-((6-(tetrahydro-2H-pyran-4-yl)pyridin-2-yl)methyl)-1,3,4-oxadiazol-2-yl)-2,6-diazaspiro[3.4]octane-6-carboxylate (150 mg, 44%) as a colorless oil. LCMS m/z=552.25 [M+H]⁺; ¹H NMR (400 MHz, Chloroform-d) δ 7.73-7.64 (m, 1H), 7.14 (d, J=7.8 Hz, 2H), 4.50-4.39 (m, 2H), 4.13-4.04 (m, 3H), 3.84-3.63 (m, 6H), 3.60-3.53 (m, 2H), 3.50-3.48 (m, 2H), 3.08-2.91 (m, 1H), 1.89-1.83 (m, 4H), 1.46 (s, 9H), 1.16-1.08 (m, 9H).

### Step 3: ((S)-2,2-dimethylcyclopropyl)(8-(5-((6-(tetrahydro-2H-pyran-4-yl)pyridin-2-yl)methyl)-1,3,4-oxadiazol-2-yl)-2,6-diazaspiro[3.4]octan-2-yl)methanone

To a solution of tert-butyl 2-((S)-2,2-dimethylcyclopropane-1-carbonyl)-8-(5-((6-(tetrahydro-2H-pyran-4-yl)pyridin-2-yl)methyl)-1,3,4-oxadiazol-2-yl)-2,6-diazaspiro[3.4]octane-6-carboxylate (150 mg, 0.27 mmol) in DCM (3 mL) was added TFA (1.5 mL) and the reaction stirred for 2 h. The solvent was removed under reduced pressure to afford ((S)-2,2-dimethylcyclopropyl)(8-(5-((6-(tetrahydro-2H-pyran-4-yl)pyridin-2-yl)methyl)-1,3,4-oxadiazol-2-yl)-2,6-diazaspiro[3.4]octan-2-yl)methanone (123 mg, 100%) which was used directly in the next step. LCMS m/z=452.2 [M+H]⁺.

### Step 4: ((S)-2,2-dimethylcyclopropyl)(8-(5-((6-(tetrahydro-2H-pyran-4-yl)pyridin-2-yl)methyl)-1,3,4-oxadiazol-2-yl)-6-(thiazolo[4,5-d]pyrimidin-7-yl)-2,6-diazaspiro[3.4]octan-2-yl)methanone

To a solution of ((S)-2,2-dimethylcyclopropyl)(8-(5-((6-(tetrahydro-2H-pyran-4-yl)pyridin-2-yl)methyl)-1,3,4-oxadiazol-2-yl)-2,6-diazaspiro[3.4]octan-2-yl)methanone (40 mg, 0.09 mmol) in MeCN was added Na₂CO₃ (29 mg, 0.27 mmol) and the reaction stirred at room temperature for 20 min. 7-chlorothiazolo[4,5-d]pyrimidine (15 mg, 0.09 mmol) was added and the reaction mixture stirred a further 6 h. The solids were removed by filtration and the filtrate concentrated and purified by prep-HPLC to afford ((S)-2,2-dimethylcyclopropyl)(8-(5-((6-(tetrahydro-2H-pyran-4-yl)pyridin-2-yl)methyl)-1,3,4-oxadiazol-2-yl)-6-(thiazolo[4,5-d]pyrimidin-7-yl)-2,6-diazaspiro[3.4]octan-2-yl)methanone (15 mg, 28%) as a white solid. LCMS m/z=587.3 [M+H]⁺; ¹H NMR (400 MHz, DMSO-d₆) δ 9.73 (s, 1H), 8.52 (s, 1H), 7.71-7.63 (m, 1H), 7.22-7.15 (m, 2H), 4.42-4.34 (m, 3H), 4.30-4.05 (m, 6H), 4.02-3.97 (m, 1H), 3.92-3.82 (m, 3H), 3.42-3.37 (m, 2H), 2.86-2.77 (m, 1H), 1.69-1.60 (m, 4H), 1.38-1.25 (m, 1H), 1.13-1.09 (m, 2H), 1.07-1.01 (m, 3H), 0.97-0.93 (m, 1H), 0.88-0.83 (m, 1H), 0.72-0.63 (m, 1H).

### Synthesis of (8-(5-((3,4-dichlorophenyl)difluoromethyl)-1,3,4-oxadiazol-2-yl)-2-((S)-2,2-dimethylcyclopropane-1-carbonyl)-2,6-diazaspiro[3.4]octan-6-yl)(pyridazin-4-yl)methanone (I-43)

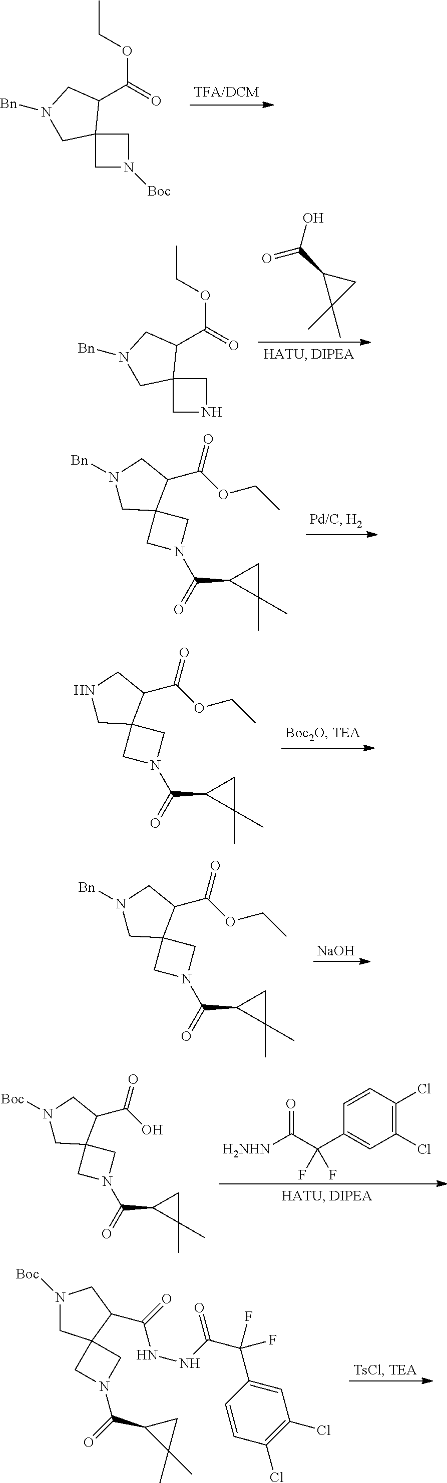

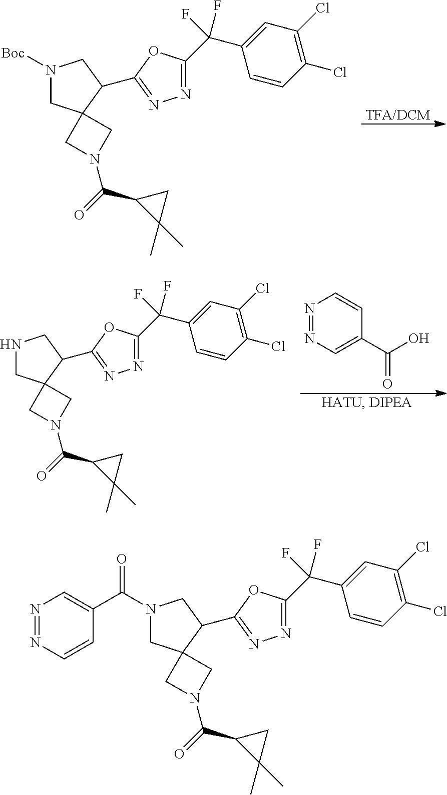

### Step 1: ethyl 6-benzyl-2,6-diazaspiro[3.4]octane-8-carboxylate

To a solution of 2-(tert-butyl) 8-ethyl 6-benzyl-2,6-diazaspiro[3.4]octane-2,8-dicarboxylate (100 mg, 0.27 mmol) in DCM (4 mL) was added TFA (1 mL). The reaction mixture was stirred at room temperature for 1 h then the solvent was removed under vacuum to afford ethyl 6-benzyl-2,6-diazaspiro[3.4]octane-8-carboxylate (73 mg, 100%) which was used directly in the next step. LCMS m/z=275.2 [M+H]⁺.

### Step 2: ethyl 6-benzyl-2-((S)-2,2-dimethylcyclopropane-1-carbonyl)-2,6-diazaspiro[3.4]octane-8-carboxylate

To a solution of (S)-2,2-dimethylcyclopropane-1-carboxylic acid (3.7 g, 13.4 mmol) in DCM (40 mL) was added HATU (5.1 g, 13.4 mmol) and DIPEA (6.9 g, 53.6 mmol). The reaction was stirred at room temperature for 30 min then ethyl 6-benzyl-2,6-diazaspiro[3.4]octane-8-carboxylate (1.5 g, 13.4 mmol) was added and the reaction stirred for another 2 h. The reaction was diluted with water (100 mL) and extracted with DCM (100 mL). The combined organic layers were washed with brine, dried over Na₂SO₄, filtered and concentrated. The residue obtained was purified by column chromatography on silica gel (eluent: DCM:MeOH=50:1) to afford ethyl 6-benzyl-2-((S)-2,2-dimethylcyclopropane-1-carbonyl)-2,6-diazaspiro[3.4]octane-8-carboxylate (4 g, 80%) as a yellow oil. LCMS m/z=371.2 [M+H]⁺; H NMR (400 MHz, DMSO-d6) 37.68-7.58 (m, 2H), 7.47-7.40 (m, 3H), 4.65-3.77 (m, 10H), 3.58-3.21 (m, 3H), 1.30 (t, J=7.2 Hz, 3H), 1.25-1.18 (m, 1H), 1.15-1.04 (m, 7H), 0.77-0.71 (m, 1H).

### Step 3: ethyl 2-((S)-2,2-dimethylcyclopropane-1-carbonyl)-2,6-diazaspiro[3.4]octane-8-carboxylate

To a solution of ethyl 6-benzyl-2-((S)-2,2-dimethylcyclopropane-1-carbonyl)-2,6-diazaspiro[3.4]octane-8-carboxylate (2 g, 5.4 mmol) in EtOAc (20 mL) was added 10% Pd/C (800 mg). The reaction mixture was heated at 40° C. under a H₂ atmosphere for 48 h then the catalyst removed by filtration through Celite and the filtrate concentrated to afford ethyl 2-((S)-2,2-dimethylcyclopropane-1-carbonyl)-2,6-diazaspiro[3.4]octane-8-carboxylate (1.7 g) which was used directly in the next step. LCMS m/z=281.2 [M+H]⁺; ¹H NMR (400 MHz, Chloroform-d) δ 4.43-4.08 (m, 5H), 3.98 (q, J=10.9 Hz, 1H), 3.82-3.64 (m, 4H), 3.44-3.37 (m, 1H), 1.33-1.24 (m, 4H), 1.16-1.09 (m, 6H), 1.08-1.00 (m, 1H), 0.80-0.74 (m, 1H).

### Step 4: 6-(tert-butyl) 8-ethyl 2-((S)-2,2-dimethylcyclopropane-1-carbonyl)-2,6-diazaspiro[3.4]octane-6,8-dicarboxylate

To a solution of ethyl 2-((S)-2,2-dimethylcyclopropane-1-carbonyl)-2,6-diazaspiro[3.4]octane-8-carboxylate (2.9 g, 10.3 mmol) in DCM (50 mL) was added TEA (2.1 g, 20.6 mmol) and (Boc)₂O (3.4 g, 15.5 mmol). The reaction mixture was stirred at room temperature for 10 h then diluted with water (30 mL), extracted with DCM (50 mL×3). The combined organic layers were washed with brine, dried over Na₂SO₄, filtered and concentrated. The residue obtained was purified by column chromatography on silica gel (eluent: DCM:MeOH=100:1) to afford 6-(tert-butyl) 8-ethyl 2-((S)-2,2-dimethylcyclopropane-1-carbonyl)-2,6-diazaspiro[3.4]octane-6,8-dicarboxylate (860 mg, 97%) as a yellow oil. LCMS m/z=381.2 [M+H]⁺; ¹H NMR (400 MHz, Chloroform-d) δ 4.33-3.83 (m, 6H), 3.77-3.49 (m, 4H), 3.07 (d, J=6.6 Hz, 1H), 1.46 (s, 9H), 1.30-1.20 (m, 4H), 1.14 (d, J=3.1 Hz, 7H), 0.74 (dd, J=8.1, 4.1 Hz, 1H).

### Step 5: 6-(tert-butoxycarbonyl)-2-((S)-2,2-dimethylcyclopropane-1-carbonyl)-2,6-diazaspiro[3.4]octane-8-carboxylic acid

To a solution of 6-(tert-butyl) 8-ethyl 2-((S)-2,2-dimethylcyclopropane-1-carbonyl)-2,6-diazaspiro[3.4]octane-6,8-dicarboxylate (2.2 g, 5.8 mmol) in a mixture of THF, water and EtOH (4.0 mL/1.0 mL/1.0 mL) was added NaOH (463 mg, 11.6 mmol). The reaction mixture was stirred at room temperature for 3 h then diluted with water (20 mL) and extracted with ether (40 mL). The aqueous layer was collected, acidified to pH˜2 with 1M HCl and extracted with EtOAc (50 mL×3). The combined organic layers were washed with brine, dried over Na₂SO₄, filtered and concentrated to afford 6-(tert-butoxycarbonyl)-2-((S)-2,2-dimethylcyclopropane-1-carbonyl)-2,6-diazaspiro[3.4]octane-8-carboxylic acid (1.9 g, 80%) as a yellow solid. LCMS m/z=353.2 [M+H]⁺; ¹H NMR (400 MHz, Chloroform-d) δ 4.41-4.22 (m, 1H), 4.17-4.03 (m, 3H), 3.97-3.87 (m, 1H), 3.78-3.50 (m, 4H), 3.10 (s, 1H), 1.46 (s, 9H), 1.25-1.11 (m, 8H), 0.82-0.73 (m, 1H).

### Step 6: tert-butyl 8-(2-(2-(3,4-dichlorophenyl)-2,2-difluoroacetyl)hydrazine-1-carbonyl)-2-((S)-2,2-dimethylcyclopropane-1-carbonyl)-2,6-diazaspiro[3.4]octane-6-carboxylate

To a solution of 6-(tert-butoxycarbonyl)-2-((S)-2,2-dimethylcyclopropane-1-carbonyl)-2,6-diazaspiro[3.4]octane-8-carboxylic acid (1 g, 2.8 mmol) in DCM (10 mL) was added HATU (1.1 g, 2.8 mmol). The reaction was stirred at room temperature for 30 min then 2-(3,4-dichlorophenyl)-2,2-difluoroacetohydrazide (714 mg, 2.8 mmol) and DIPEA (1.1 g, 8.4 mmol) were added and the reaction stirred for further 3 h. The mixture was diluted with water (20 mL) and extracted with DCM (50 mL×3). The combined organic layers were washed with brine, dried over Na₂SO₄, filtered and concentrated. The residue obtained was purified by column chromatography on silica gel (eluent: DCM:MeOH=50:1) to afford tert-butyl (S)-6-(2,4-dimethylthiazole-5-carbonyl)-8-((R)-2-oxo-4-phenyloxazolidine-3-carbonyl)-2,6-diazaspiro[3.4]octane-2-carboxylate (1.5 g, 90%) as a yellow solid. LCMS m/z=589.1 [M+H]⁺; 1H NMR (400 MHz, DMSO-d6) δ 11.11 (s, 1H), 10.38 (s, 1H), 7.95-7.83 (m, 2H), 7.63 (dd, J=8.4, 2.0 Hz, 1H), 4.22-3.92 (m, 2H), 3.90-3.34 (m, 6H), 3.18-3.05 (m, 1H), 1.39 (s, 9H), 1.36-1.28 (m, 1H), 1.14-0.99 (m, 6H), 0.89-0.81 (m, 1H), 0.72-0.63 (m, 1H).

### Step 7: tert-butyl 8-(5-((3,4-dichlorophenyl)difluoromethyl)-1,3,4-oxadiazol-2-yl)-2-((S)-2,2-dimethylcyclopropane-1-carbonyl)-2,6-diazaspiro[3.4]octane-6-carboxylate

To a solution of tert-butyl 8-(2-(2-(3,4-dichlorophenyl)-2,2-difluoroacetyl)hydrazine-1-carbonyl)-2-((S)-2,2-dimethylcyclopropane-1-carbonyl)-2,6-diazaspiro[3.4]octane-6-carboxylate (1.2 g, 2 mmol) in DCM (10 mL) was added TEA (1 g, 10 mmol) and TsCl (1.1 g, 6 mmol). The reaction mixture was stirred at room temperature for 2 h then diluted with water (30 mL), extracted with DCM (50 mL×3). The combined organic layers were washed with brine, dried over Na₂SO₄, filtered and concentrated. The residue obtained was purified by column chromatography on silica gel (eluent: DCM:MeOH=50:1) to afford tert-butyl 8-(5-((3,4-dichlorophenyl)difluoromethyl)-1,3,4-oxadiazol-2-yl)-2-((S)-2,2-dimethylcyclopropane-1-carbonyl)-2,6-diazaspiro[3.4]octane-6-carboxylate (900 mg, 78%) as a colourless oil. LCMS m/z=571.1 [M+H]⁺; ¹H NMR (400 MHz, Chloroform-d) δ 7.77 (s, 1H), 7.61 (d, J=8.5 Hz, 1H), 7.49 (d, J=7.8 Hz, 1H), 4.31-3.98 (m, 3H), 3.94-3.69 (m, 6H), 1.47 (s, 9H), 1.21-1.05 (m, 8H), 0.80-0.70 (m, 1H).

### Step 8: (8-(5-((3,4-dichlorophenyl)difluoromethyl)-1,3,4-oxadiazol-2-yl)-2,6-diazaspiro[3.4]octan-2-yl)((S)-2,2-dimethylcyclopropyl)methanone

To a solution of tert-butyl 8-(5-((3,4-dichlorophenyl)difluoromethyl)-1,3,4-oxadiazol-2-yl)-2-((S)-2,2-dimethylcyclopropane-1-carbonyl)-2,6-diazaspiro[3.4]octane-6-carboxylate (50 mg, 0.1 mmol) in DCM (2 mL) was added TFA (0.5 mL) and the reaction stirred at room temperature for 1 h. The solvent was removed under reduced pressure to afford (8-(5-((3,4-dichlorophenyl)difluoromethyl)-1,3,4-oxadiazol-2-yl)-2,6-diazaspiro[3.4]octan-2-yl)((S)-2,2-dimethylcyclopropyl)methanone (41 mg, 100%) which was used directly in the next step. LCMS m/z=471 [M+H]f.

### Step 9: (8-(5-((3,4-dichlorophenyl)difluoromethyl)-1,3,4-oxadiazol-2-yl)-2-((S)-2,2-dimethylcyclopropane-1-carbonyl)-2,6-diazaspiro[3.4]octan-6-yl)(pyridazin-4-yl)methanone

To a solution of pyridazine-4-carboxylic acid (11 mg, 0.09 mmol) in DCM (3 mL) was added HATU (34 mg, 0.09 mmol) and DIPEA (46 mg, 0.36 mmol). The mixture was stirred at room temperature for 30 min then (8-(5-((3,4-dichlorophenyl)difluoromethyl)-1,3,4-oxadiazol-2-yl)-2,6-diazaspiro[3.4]octan-2-yl)((S)-2,2-dimethylcyclopropyl)methanone (41 mg, 0.09 mmol) was added and the reaction stirred at room temperature for another 2 h. The rection was diluted with water (10 mL) and extracted with DCM (50 mL), the combined organic layers were washed with brine, dried over Na₂SO₄, filtered and concentrated. The residue obtained was purified by prep-HPLC to afford (8-(5-((3,4-dichlorophenyl)difluoromethyl)-1,3,4-oxadiazol-2-yl)-2-((S)-2,2-dimethylcyclopropane-1-carbonyl)-2,6-diazaspiro[3.4]octan-6-yl)(pyridazin-4-yl)methanone I-43 (30 mg, 60%) as a white solid. LCMS m/z=577.2 [M+H]⁺; ¹H NMR (400 MHz, DMSO-d6) δ 9.39 (d, J=5.2 Hz, 1H), 9.34 (d, J=6.8 Hz, 1H), 8.06-7.95 (m, 1H), 7.93-7.79 (m, 2H), 7.77-7.66 (m, 1H), 4.39-3.72 (m, 9H), 1.40-1.19 (m, 1H), 1.15-0.89 (m, 6H), 0.88-0.78 (m, 1H), 0.73-0.60 (m, 1H).

Table 13: The compounds listed in Table 13 were synthesized from (8-(5-((3,4-dichlorophenyl)difluoromethyl)-1,3,4-oxadiazol-2-yl)-2,6-diazaspiro[3.4]octan-2-yl)((S)-2,2-dimethylcyclopropyl)methanone according to the procedures outlined for I-43 using the appropriate commercially available reagents and/or intermediates described elsewhere.

TABLE 13

| Example | | | |
|---|---|---|---|
| Number | Compound | ¹HNMR | LCMS |
| I-45 | 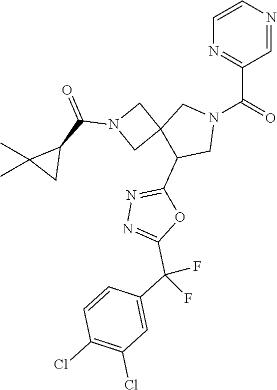 [SMILES: CC1(C)C[C@@H]1C(=O)N1CC2(CN(C(=O)c3cnccn3)CC2c2nnc(C(F)(F)c3ccc(Cl)c(Cl)c3)o2)C1] | ¹H NMR (400 MHz, Methanol-d₄) δ 9.11- 9.03 (m, 1H), 8.76-8.62 (m, 2H), 7.93-7.83 (m, 1H), 7.77-7.70 (m, 1H), 7.62 (ddt, J = 11.2, 7.8, 3.4 Hz, 1H), 4.35 (s, 4H), 4.08 (d, J = 72.0 Hz, 5H), 1.38 (s, 1H), 1.20-0.99 (m, 7H), 0.75 (s, 1H). | m/z = 577.1 [M + H]⁺ |
| I-56 | 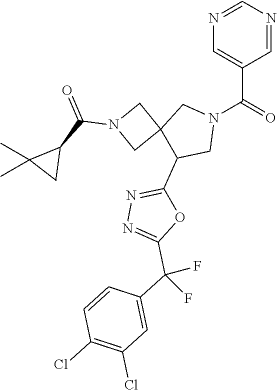 [SMILES: CC1(C)C[C@@H]1C(=O)N1CC2(CN(C(=O)c3cncnc3)CC2c2nnc(C(F)(F)c3ccc(Cl)c(Cl)c3)o2)C1] | ¹H NMR (400 MHz, Methanol-d4) δ 9.26 (s, 1H), 9.01 (d, J = 3.0 Hz, 2H), 7.89 (d, J = 15.8 Hz, 1H), 7.75 (s, 1H), 7.68- 7.58 (m, 1H), 4.56-4.34 (m, 1H), 4.32-3.82 (m, 8H), 1.47-1.27 (m, 1H), 1.22-0.96 (m, 7H), 0.83- 0.70 (m, 1H). | m/z = 577.2 [M + H]⁺ |
| I-45 | 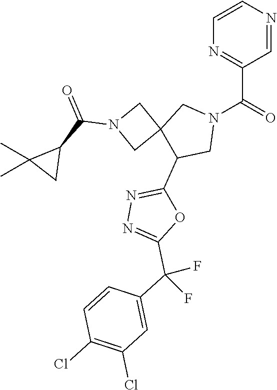 [SMILES: CC1(C)C[C@@H]1C(=O)N1CC2(CN(C(=O)c3cnccn3)CC2c2nnc(C(F)(F)c3ccc(Cl)c(Cl)c3)o2)C1] | ¹HNMR (DMSO, 400 MHz) δ 9.01 (1H, s), 8.80 (1H, s), 8.71 (1H, s), 8.03 (1H, s), 7.89 (1H, s), 7.74 (1H, s), 3.72-4.40 (9H, m), 1.21-1.38 (1H, m), 0.91-1.16 (6H, m), 0.82 (1H, s), 0.64 (1H, s). | m/z = 577.2 [M + H]⁺ |
| I-46 | 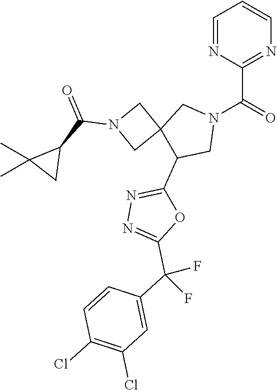 [SMILES: CC1(C)C[C@@H]1C(=O)N1CC2(CN(C(=O)c3ncccn3)CC2c2nnc(C(F)(F)c3ccc(Cl)c(Cl)c3)o2)C1] | ¹H NMR (400 MHz, DMSO-d₆) δ 8.93 (t, J = 5.2 Hz, 2H), 8.05-7.96 (m, 1H), 7.90 (m, 1H), 7.71 (dd, J= 18.8, 8.6 Hz, 1H), 7.63 (td, J = 4.8, 3.2 Hz, 1H), 4.40- 4.07 (m, 3H), 4.02-3.77 (m, 6H), 1.37-1.22 (m, 1H), 1.14-1.02 (m, 4H), 0.98 (d, J = 3.2 Hz, 1H), 0.91 (s, 1H), 0.83 (m, 1H), 0.70-0.61 (m, 1H). | m/z = 577.2 [M + H]⁺ |
| I-53 | 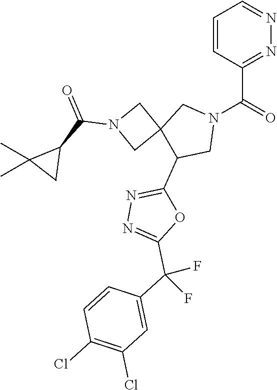 [SMILES: CC1(C)C[C@@H]1C(=O)N1CC2(CN(C(=O)c3cccnn3)CC2c2nnc(C(F)(F)c3ccc(Cl)c(Cl)c3)o2)C1] | ¹H NMR (400 MHz, DMSO-d₆) δ 9.35 (s, 1H), 8.06-7.96 (m, 2H), 7.92-7.85 (m, 2H), 7.76- 7.67 (m, 1H), 4.42- 4.09 (m, 5H), 4.08-3.92 (m, 3H), 3.90-3.73 (m, 1H), 1.41-1.20 (m, 1H), 1.15-1.02 (m, 4H), 0.99- 0.90 (m, 2H), 0.88- 0.79 (m, 1H), 0.72-0.60 (m, 1H). | m/z = 577.2 [M + H]⁺ |
| I-44 | 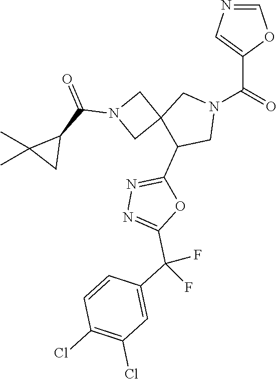 [SMILES: CC1(C)C[C@@H]1C(=O)N1CC2(CN(C(=O)c3cnco3)CC2c2nnc(C(F)(F)c3ccc(Cl)c(Cl)c3)o2)C1] | ¹H NMR (400 MHz, DMSO-d₆) δ 8.60 (s, 1H), 8.01 (s, 1H), 7.91- 7.78 (m, 2H), 7.72 (d, J = 7.6 Hz, 1H), 4.28-4.08 (m, 5H), 4.00-3.82 (m, 4H), 1.35-1.23 (m, 1H), 1.10 (d, J = 8.2 Hz, 2H), 1.05 (s, 3H), 0.99 (d, J = 6.0 Hz, 1H), 0.94 (s, 1H), 0.85 (s, 1H), 0.67 (s, 1H). | m/z = 566.2 [M + H]⁺ |
| I-52 | 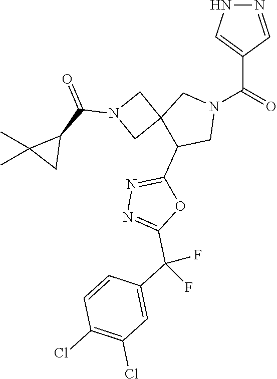 [SMILES: CC1(C)C[C@@H]1C(=O)N1CC2(CN(C(=O)c3cn[nH]c3)CC2c2nnc(C(F)(F)c3ccc(Cl)c(Cl)c3)o2)C1] | ¹H NMR (400 MHz, DMSO-d₆) δ 13.32- 13.21 (m, 1H), 8.25- 8.15 (m, 1H), 8.05-7.98 (m, 1H), 7.92-7.80 (m, 2H), 7.76-7.65 (m, 1H), 4.35-4.06 (m, 5H), 4.02- 3.79 (m, 4H), 1.40- 1.21 (m, 1H), 1.13-1.08 (m, 2H), 1.06-1.03 (m, 2H), 1.01-0.97 (m, 1H), 0.95-0.91 (m, 1H), 0.88- 0.80 (m, 1H), 0.73- 0.61 (m, 1H). | m/z = 565.2 [M + H]⁺ |
| I-54 | 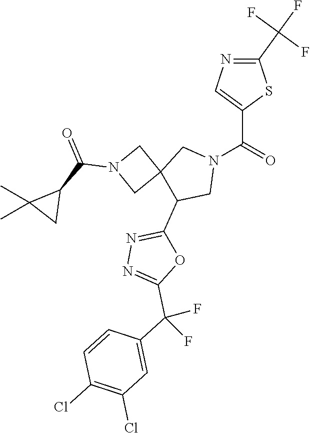 [SMILES: CC1(C)C[C@@H]1C(=O)N1CC2(CN(C(=O)c3cnc(C(F)(F)F)s3)CC2c2nnc(C(F)(F)c3ccc(Cl)c(Cl)c3)o2)C1] | ¹H NMR (400 MHz, DMSO-d₆) δ 8.62-8.51 (m, 1H), 8.05-7.96 (m, 1H), 7.92-7.83 (m, 1H), 7.76-7.66 (m, 1H), 4.34- 3.92 (m, 8H), 3.90- 3.81 (m, 1H), 1.37-1.25 (m, 1H), 1.13-0.94 (m, 6H), 0.88-0.82 (m, 1H), 0.73- 0.63 (m, 1H). | m/z = 650.2 [M + H]⁺ |

### Synthesis of (8-(5-((4-chlorophenyl)difluoromethyl)-1,3,4-oxadiazol-2-yl)-6-(thiazolo[4,5-d]pyrimidin-7-yl)-2,6-diazaspiro[3.4]octan-2-yl)((S)-2,2-dimethylcyclopropyl)methanone (I-37)

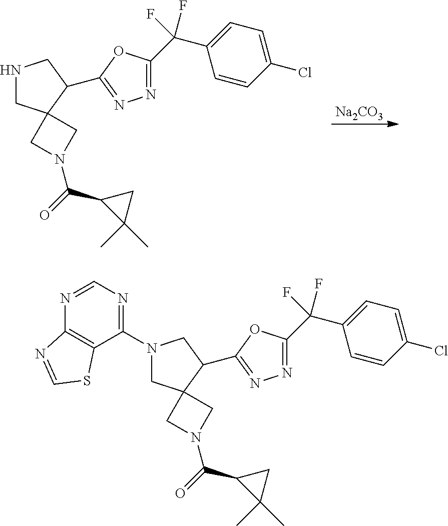

### (8-(5-((4-chlorophenyl)difluoromethyl)-1,3,4-oxadiazol-2-yl)-2,6-diazaspiro[3.4]octan-2-yl)((S)-2,2-dimethylcyclopropyl)methanone

(8-(5-((4-chlorophenyl)difluoromethyl)-1,3,4-oxadiazol-2-yl)-2,6-diazaspiro[3.4]octan-2-yl)((S)-2,2-dimethylcyclopropyl)methanone was synthesized from 6-(tert-butoxycarbonyl)-2-((S)-2,2-dimethylcyclopropane-1-carbonyl)-2,6-diazaspiro[3.4]octane-8-carboxylic acid according to the procedures outlined for (8-(5-((3,4-dichlorophenyl)difluoromethyl)-1,3,4-oxadiazol-2-yl)-2,6-diazaspiro[3.4]octan-2-yl)((S)-2,2-dimethylcyclopropyl)methanone using the appropriate commercially available reagents and/or intermediates described elsewhere. LCMS m/z=437.1 [M+H]⁺.

### Step 1: (8-(5-((4-chlorophenyl)difluoromethyl)-1,3,4-oxadiazol-2-yl)-6-(thiazolo[4,5-d]pyrimidin-7-yl)-2,6-diazaspiro[3.4]octan-2-yl)((S)-2,2-dimethylcyclopropyl)methanone

To a solution of (8-(5-((4-chlorophenyl)difluoromethyl)-1,3,4-oxadiazol-2-yl)-2,6-diazaspiro[3.4]octan-2-yl)((S)-2,2-dimethylcyclopropyl)methanone (70 mg, 0.16 mmol, 1.0 eq.) in DMF (2 mL) was added 7-chlorothiazolo[4,5-d]pyrimidine (30 mg, 0.17 mmol, 1.1 eq) and Na₂CO₃ (34 mg, 0.32 mmol, 2.0 eq.). The reaction was stirred at room temperature overnight then diluted with water (30 mL) and extracted with EtOAc (50 mL×3). The combined organic layers were washed with brine, dried over Na₂SO₄, filtered and concentrated. The residue was purified by prep-HPLC to afford (8-(5-((4-chlorophenyl)difluoromethyl)-1,3,4-oxadiazol-2-yl)-6-(thiazolo[4,5-d]pyrimidin-7-yl)-2,6-diazaspiro[3.4]octan-2-yl)((S)-2,2-dimethylcyclopropyl)methanone (6 mg, 7%) as a white solid. LCMS m/z=572.2 [M+H]⁺; ¹H NMR (400 MHz, CDCl₃) δ 9.30 (s, 1H), 8.68 (s, 1H), 7.57 (d, J=8.4 Hz, 2H), 7.48 (d, J=8.4 Hz, 2H), 4.45-3.95 (m, 9H), 1.20-1.05 (m, 8H), 0.81-0.75 (m, 1H).

### Synthesis of (6-(benzo[d]thiazol-7-yl)-8-(5-((3,4-dichlorophenyl)difluoromethyl)-1,3,4-oxadiazol-2-yl)-2,6-diazaspiro[3.4]octan-2-yl)((S)-2,2-dimethylcyclopropyl)methanone (I-98)

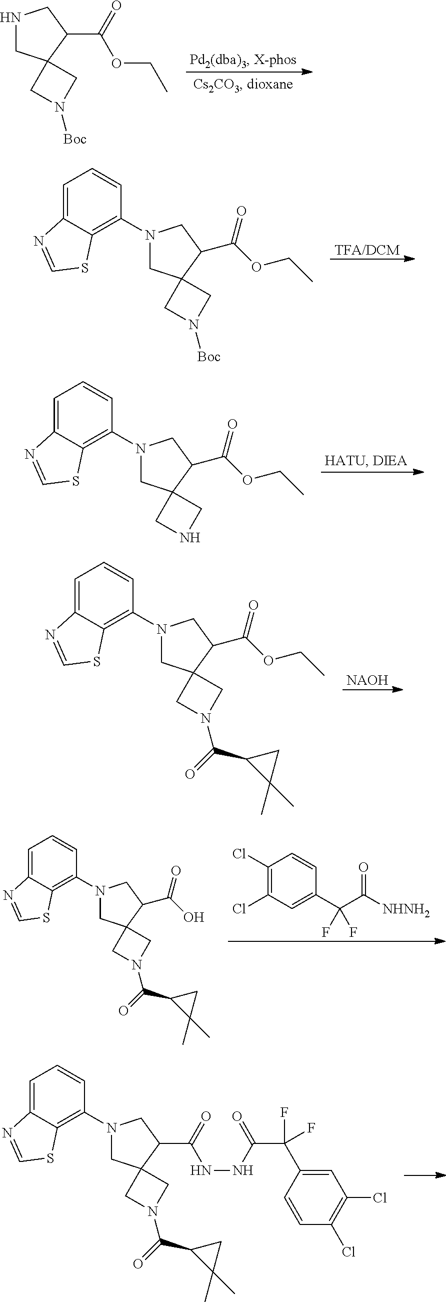

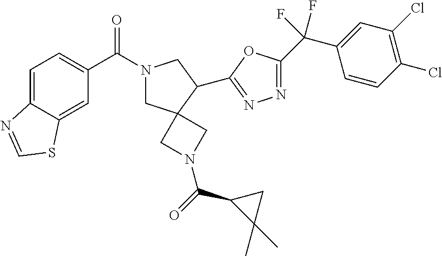 [SMILES: CC1(C)C[C@@H]1C(=O)N1CC2(CN(C(=O)c3ccc4ncsc4c3)CC2c2nnc(C(F)(F)c3ccc(Cl)c(Cl)c3)o2)C1]

### Step 1: 2-(tert-butyl) 8-ethyl 6-(benzo[d]thiazol-7-yl)-2,6-diazaspiro[3.4]octane-2,8-dicarboxylate

To a solution of 2-(tert-butyl) 8-ethyl 2,6-diazaspiro[3.4]octane-2,8-dicarboxylate (500 mg, 1.76 mmol, 1.0 eq.) in dioxane (3 mL) was added 7-bromobenzo[d]thiazole (410 mg, 1.98 mmol, 1.1 eq.), Pd₂(dba)₃ (100 mg, 0.18 mmol, 0.1 eq.), X-phos (165 mg, 0.35 mmol, 0.2 eq.) and Cs₂CO₃ (1.14 g, 3.52 mmol, 2.0 eq.). The resulting mixture was stirred under N₂ atmosphere at 90° C. overnight. The mixture was diluted with water (30 mL), extracted with EtOAc (50 mL×2). The combined organic layers was washed with brine, dried over Na₂SO₄, filtered and concentrated. The residue was purified by silica gel chromatography (eluent: Pet. Ether:EtOAc=10:1 to 3:1) to afford the 2-(tert-butyl) 8-ethyl 6-(benzo[d]thiazol-7-yl)-2,6-diazaspiro[3.4]octane-2,8-dicarboxylate (500 mg, 68.1%) as a white solid. LCMS m/z=418.1 [M+H]⁺; ¹H NMR (400 MHz, CD₃OD) δ 9.15 (s, 1H), 7.50 (d, J=8.0 Hz, 1H), 7.40 (t, J=7.9 Hz, 1H), 6.70 (d, J=7.9 Hz, 1H), 4.35-4.14 (m, 3H), 4.08-3.78 (m, 8H), 3.42 (dd, J=7.8, 6.3 Hz, 1H), 1.47 (s, 9H), 1.31 (t, J=7.2 Hz, 4H).

### Step 2: ethyl 6-(benzo[d]thiazol-7-yl)-2,6-diazaspiro[3.4]octane-8-carboxylate

A mixture of 2-(tert-butyl) 8-ethyl 6-(benzo[d]thiazol-7-yl)-2,6-diazaspiro[3.4]octane-2,8-dicarboxylate (500 mg, 1.2 mmol, 1.0 eq.) in TFA/DCM (1/3, 4 mL) was stirred at room temperature for 3 h. The solvent was concentrated to afford crude ethyl 6-(benzo[d]thiazol-7-yl)-2,6-diazaspiro[3.4]octane-8-carboxylate (380 mg, quant.) as a yellow oil. LCMS m/z=318.1 [M+H]⁺.

### Step 3: ethyl 6-(benzo[d]thiazol-7-yl)-2-((S)-2,2-dimethylcyclopropane-1-carbonyl)-2,6-diazaspiro[3.4]octane-8-carboxylate

To a solution of (S)-2,2-dimethylcyclopropane-1-carboxylic acid (150 mg, 1.3 mmol, 1.1 eq.) in DCM (6 mL) was added HATU (683 mg, 1.8 mmol, 1.5 eq.) and DIEA (464 mg, 3.6 mmol, 3.0 eq). The resulting mixture was stirred at room temperature for 0.5 h. Then ethyl 6-(benzo[d]thiazol-7-yl)-2,6-diazaspiro[3.4]octane-8-carboxylate (380 mg, 1.2 mmol, 1.0 eq) was added. The mixture was stirred at room temperature overnight. The mixture was diluted with water (20 mL), extracted with EtOAc (30 mL×3). The combined organic layers was washed with brine, dried over Na₂SO₄, filtered and concentrated. The residue was purified by column chromatography on silica gel (eluent: Pet. Ether:EtOAc=10:1 to 1:1) to afford N′-(2-(4-cyclopropylphenyl)-2,2-difluoroacetyl)-2-((S)-2,2-dimethylcyclopropane-1-carbonyl)-6-(thiazole-5-carbonyl)-2,6-diazaspiro[3.4]octane-8-carbohydrazide (300 mg, 60.6%) as a yellow solid. LCMS m/z=414.2 [M+H]⁺; ¹H NMR (400 MHz, CD₃OD) δ 9.14 (s, 1H), 7.50 (d, J=8.1 Hz, 1H), 7.40 (t, J=8.0 Hz, 1H), 6.70 (d, J=7.9 Hz, 1H), 4.54-3.80 (m, 10H), 3.52-3.41 (m, 1H), 1.51-1.42 (m, 1H), 1.29 (td, J=7.1, 4.2 Hz, 4H), 1.18 (dd, J=16.9, 7.5 Hz, 6H), 1.06 (q, J=4.3, 3.8 Hz, 1H), 0.80 (dq, J=8.2, 4.3 Hz, 1H).

### Step 4: 6-(benzo[d]thiazol-7-yl)-2-((S)-2,2-dimethylcyclopropane-1-carbonyl)-2,6-diazaspiro[3.4]octane-8-carboxylic acid

To a solution of ethyl 6-(benzo[d]thiazol-7-yl)-2-((S)-2,2-dimethylcyclopropane-1-carbonyl)-2,6-diazaspiro[3.4]octane-8-carboxylate (300 mg, 0.72 mmol, 1.0 eq.) in MeOH (2 mL) was added aq. NaOH (1 M, 1 mL). The mixture was stirred at room temperature for 2 h, diluted with water (20 mL) and extracted with ether (40 mL). The aqueous layer was collected and acidified to pH˜2 with 1M HCl and extracted with EtOAc (60 mL×3). The combined organic layers was washed with brine, dried over Na₂SO₄, filtered and concentrated to afford 6-(benzo[d]thiazol-7-yl)-2-((S)-2,2-dimethylcyclopropane-1-carbonyl)-2,6-diazaspiro[3.4]octane-8-carboxylic acid (279 mg, quant.) as a yellow oil.

### Step 5: 6-(benzo[d]thiazol-7-yl)-N′-(2-(3,4-dichlorophenyl)-2,2-difluoroacetyl)-2-((S)-2,2-dimethylcyclopropane-1-carbonyl)-2,6-diazaspiro[3.4]octane-8-carbohydrazide

To a solution of 6-(benzo[d]thiazol-7-yl)-2-((S)-2,2-dimethylcyclopropane-1-carbonyl)-2,6-diazaspiro[3.4]octane-8-carboxylic acid (280 mg, 0.72 mmol, 1.0 eq) in DCM (5 mL) was added HATU (414 mg, 1.1 mmol, 1.5 eq) and DIEA (281 mg, 2.2 mmol, 3.0 eq). The resulting mixture was stirred at room temperature for 0.5 h. Then 2-(3,4-dichlorophenyl)-2,2-difluoroacetohydrazide (180 mg, 0.8 mmol, 1.1 eq.) was added. The mixture was stirred at room temperature overnight. The mixture was diluted with water (30 mL), extracted with EtOAc (50 mL×2). The combined organic layers was washed with brine, dried over Na₂SO₄, filtered and concentrated. The residue was purified by column chromatography on silica gel (eluent: DCM/MeOH=20:1 to 15:1) to afford 6-(benzo[d]thiazol-7-yl)-N′-(2-(3,4-dichlorophenyl)-2,2-difluoroacetyl)-2-((S)-2,2-dimethylcyclopropane-1-carbonyl)-2,6-diazaspiro[3.4]octane-8-carbohydrazide (230 mg, 60.8%) as a yellow solid. LCMS m/z=622.0 [M+H]⁺.

### Step 6: (6-(benzo[d]thiazol-7-yl)-8-(5-((3,4-dichlorophenyl)difluoromethyl)-1,3,4-oxadiazol-2-yl)-2,6-diazaspiro[3.4]octan-2-yl)((S)-2,2-dimethylcyclopropyl)methanone

To a solution of 6-(benzo[d]thiazol-7-yl)-N′-(2-(3,4-dichlorophenyl)-2,2-difluoroacetyl)-2-((S)-2,2-dimethylcyclopropane-1-carbonyl)-2,6-diazaspiro[3.4]octane-8-carbohydrazide (100 mg, 0.16 mmol, 1.0 eq) in DCM (5 mL) was added TsCl (94 mg, 0.48 mmol, 3.0 eq) and TEA (49 mg, 0.48 mmol, 3.0 eq). The resulting mixture was stirred at room temperature overnight. The mixture was diluted with water (15 mL), extracted with EtOAc (30 mL×2). The combined organic layers was washed with brine, dried over Na₂SO₄, filtered and concentrated. The residue was purified by prep-TLC (eluent: DCM/MeOH=15:1) to afford (6-(benzo[d]thiazol-7-yl)-8-(5-((3,4-dichlorophenyl)difluoromethyl)-1,3,4-oxadiazol-2-yl)-2,6-diazaspiro[3.4]octan-2-yl)((S)-2,2-dimethylcyclopropyl)methanone (25 mg, 61%) as a white solid. LCMS m/z=604.2 [M+H]⁺; ¹H NMR (400 MHz, CD₃OD) δ 9.14 (s, 1H), 7.85 (d, J=2.4 Hz, 1H), 7.71-7.65 (m, 1H), 7.58 (dp, J=9.6, 3.5, 2.8 Hz, 1H), 7.52 (d, J=8.1 Hz, 1H), 7.40 (td, J=8.1, 1.6 Hz, 1H), 6.72 (d, J=7.8 Hz, 1H), 5.48 (s, 1H), 4.54-3.94 (m, 9H), 1.47-1.37 (m, 1H), 1.34-1.25 (m, 1H), 1.21-1.02 (m, 6H), 0.78 (m, J=14.4, 8.1, 4.3 Hz, 1H).

### Synthesis of (6-(benzo[d]thiazol-7-yl)-8-(5-(3,4-dichlorobenzyl)-1,3,4-oxadiazol-2-yl)-2,6-diazaspiro[3.4]octan-2-yl)((S)-2,2-dimethylcyclopropyl)methanone (I-101)

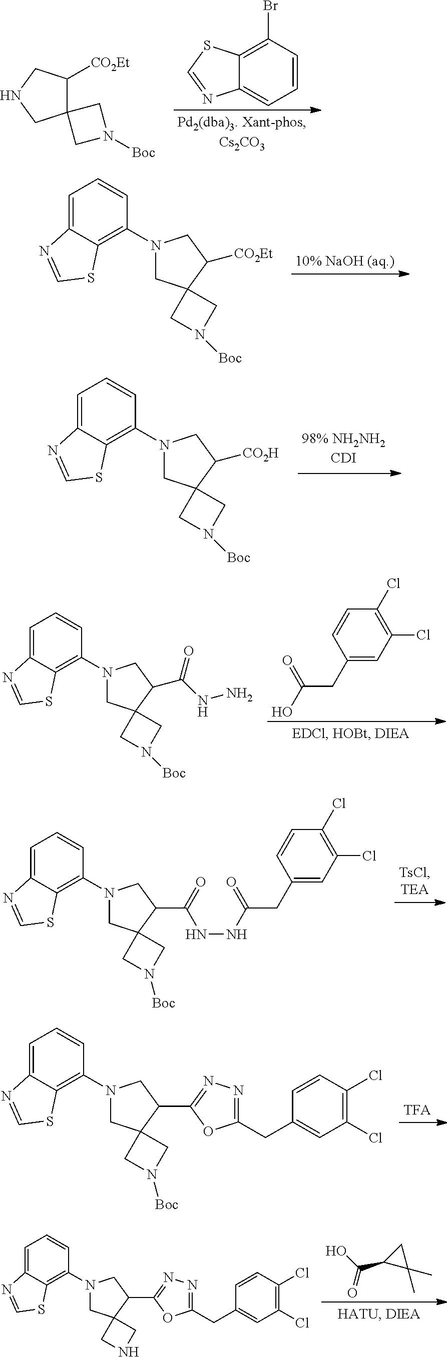

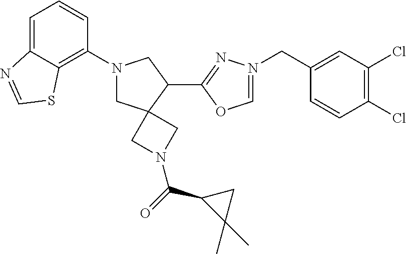

### Step 1: 2-(tert-butyl) 8-ethyl 6-(benzo[d]thiazol-7-yl)-2,6-diazaspiro[3.4]octane-2,8-dicarboxylate

A mixture of 2-(tert-butyl) 8-ethyl 2,6-diazaspiro[3.4]octane-2,8-dicarboxylate (954 mg, 3.34 mmol), 7-bromobenzo[d]thiazole (790 mg, 3.67 mmol), Pd₂(dba)₃ (311 mg, 0.34 mmol), Xant-phos (393 mg, 0.68 mmol) and Cs₂CO₃ (2.2 g, 6.8 mmol) in dioxane (10.0 mL) was stirred under N₂ at 100° C. overnight. The mixture was diluted with water (30 mL), extracted with EtOAc (50 mL×3). The combined organic layers was washed with brine, dried over Na₂SO₄, filtered and concentrated. The residue was purified by column chromatography on silica gel (eluent: Pet. ether/EtOAc=10/1 to 7/1) to afford 2-(tert-butyl) 8-ethyl 6-(benzo[d]thiazol-7-yl)-2,6-diazaspiro[3.4]octane-2,8-dicarboxylate (635 mg, 45% yield) as a yellow oil. LCMS m/z=256.2 [M+H]⁺; ¹H NMR (400 MHz, DMSO-d₆) δ 4.66-4.47 (m, 4H), 4.10 (q, J=7.1 Hz, 2H), 1.67-1.65 (m, 3H), 1.39 (s, 9H), 1.21 (t, J=7.1 Hz, 3H).

### Step 2: 6-(benzo[d]thiazol-7-yl)-2-(tert-butoxycarbonyl)-2,6-diazaspiro[3.4]octane-8-carboxylic acid

To a solution of 2-(tert-butyl) 8-ethyl 6-(benzo[d]thiazol-7-yl)-2,6-diazaspiro[3.4]octane-2,8-dicarboxylate (630 mg, 1.5 mmol) and in MeOH (2.0 mL) was added 10% aq. NaOH (8.0 mL). The resulting solution was stirred at room temperature for 4 h. The mixture was stirred at room temperature for 2 h, diluted with water (20 mL) and extracted with ether (50 mL). The aqueous layer was collected and acidified to pH˜2 with 1M HCl and extracted with EtOAc (100 mL×3). The combined organic layers was washed with brine, dried over Na₂SO₄, filtered and concentrated to afford 6-(benzo[d]thiazol-7-yl)-2-(tert-butoxycarbonyl)-2,6-diazaspiro[3.4]octane-8-carboxylic acid (588 mg, quant.) as a white solid. LCMS m/z=389.2 [M+H]⁺.

### Step 3: tert-butyl: 6-(benzo[d]thiazol-7-yl)-8-(hydrazinecarbonyl)-2,6-diazaspiro[3.4]octane-2-carboxylate (A-0835-3)

To a solution of 6-(benzo[d]thiazol-7-yl)-2-(tert-butoxycarbonyl)-2,6-diazaspiro[3.4]octane-8-carboxylic acid (488 mg, 1.25 mmol) in THF (1.0 mL) was added CDI (244 mg, 1.5 mmol) and the reaction stirred for 30 min. Hydrazine (98% 188 mg, 3.76 mmol) was added and the resulting solution stirred at room temperature overnight. The mixture was diluted with water (30 mL), extracted with EtOAc (100 mL×3), the combined organic layers were washed with brine, dried over Na₂SO₄, filtered and concentrated to afford tert-butyl 6-(benzo[d]thiazol-7-yl)-8-(hydrazinecarbonyl)-2,6-diazaspiro[3.4]octane-2-carboxylate (432 mg, 85% yield) as a white solid. LCMS m/z=629.4 [M+H]⁺.

### Step 4: tert-butyl 6-(benzo[d]thiazol-7-yl)-8-(2-(2-(3,4-dichlorophenyl)acetyl)hydrazine-1-carbonyl)-2,6-diazaspiro[3.4]octane-2-carboxylate

To a solution of tert-butyl 6-(benzo[d]thiazol-7-yl)-8-(hydrazinecarbonyl)-2,6-diazaspiro[3.4]octane-2-carboxylate (432 mg, 1.07 mmol) in DMF (6.0 mL) was added 2-(3,4-dichlorophenyl)acetic acid (264 mg, 1.29 mmol), EDCI (308 mg, 1.61 mmol), HOBt (174 mg, 1.29 mmol) and DIPEA (554 mg, 4.29 mmol). The reaction was stirred at room temperature overnight then diluted with water (30 mL) and extracted with EtOAc (100 mL×3). The combined organic layers were washed with brine, dried over Na₂SO₄, filtered and concentrated. The residue was purified by column chromatography on silica gel (eluent: DCM/MeOH=30/1 to 10/1) to afford tert-butyl 6-(benzo[d]thiazol-7-yl)-8-(2-(2-(3,4-dichlorophenyl)acetyl)hydrazine-1-carbonyl)-2,6-diazaspiro[3.4]octane-2-carboxylate (580 mg, 92% yield) as a white solid. LCMS m/z=564.3 [M+H]⁺.

### Step 5: tert-butyl 6-(benzo[d]thiazol-7-yl)-8-(5-(3,4-dichlorobenzyl)-1,3,4-oxadiazol-2-yl)-2,6-diazaspiro[3.4]octane-2-carboxylate

To a mixture of tert-butyl 6-(benzo[d]thiazol-7-yl)-8-(2-(2-(3,4-dichlorophenyl)acetyl)hydrazine-1-carbonyl)-2,6-diazaspiro[3.4]octane-2-carboxylate (580 mg, 0.98 mmol) in DCM (6.0 mL) was added triethylamine (299 mg, 2.95 mmol) and TsCl (282 mg, 1.48 mmol). The reaction was stirred at room temperature overnight then diluted with water (30 mL) and extracted with EtOAc (50 mL×3). The combined organic layers were washed with brine, dried over Na₂SO₄, filtered and concentrated. The residue was purified by column chromatography on silica gel (eluent: DCM/MeOH=30/1 to 10/1) to afford tert-butyl 6-(benzo[d]thiazol-7-yl)-8-(5-(3,4-dichlorobenzyl)-1,3,4-oxadiazol-2-yl)-2,6-diazaspiro[3.4]octane-2-carboxylate (270 mg, 48% yield) as a white solid. LCMS m/z=564.3 [M+H]⁺.

### Step 6: 2-(6-(benzo[d]thiazol-7-yl)-2,6-diazaspiro[3.4]octan-8-yl)-5-(3,4-dichlorobenzyl)-1,3,4-oxadiazole

To a solution of tert-butyl 6-(benzo[d]thiazol-7-yl)-8-(5-(3,4-dichlorobenzyl)-1,3,4-oxadiazol-2-yl)-2,6-diazaspiro[3.4]octane-2-carboxylate (150 mg, 0.26 mmol) in DCM (2.0 mL) was added TFA (0.5 mL). The reaction was stirred for 3 h then the solvent was removed under reduced pressure to afford 2-(6-(benzo[d]thiazol-7-yl)-2,6-diazaspiro[3.4]octan-8-yl)-5-(3,4-dichlorobenzyl)-1,3,4-oxadiazole (168 mg, quant.) as a yellow oil. LCMS m/z=472.1 [M+H]⁺.

### Step 7: (6-(benzo[d]thiazol-7-yl)-8-(5-(3,4-dichlorobenzyl)-1,3,4-oxadiazol-2-yl)-2,6-diazaspiro[3.4]octan-2-yl)((S)-2,2-dimethylcyclopropyl)methanone

To a solution of (S)-2,2-dimethylcyclopropane-1-carboxylic acid (49 mg, 0.43 mmol) in DCM (5.0 mL) was added HATU (163 mg, 0.43 mmol) and DIPEA (554 mg, 4.29 mmol). The reaction was stirred at room temperature for 30 min then (6-(benzo[d]thiazol-7-yl)-8-(5-(3,4-dichlorobenzyl)-1,3,4-oxadiazol-2-yl)-2,6-diazaspiro[3.4]octan-2-yl)((S)-2,2-dimethylcyclopropyl)methanone (168 mg, 0.36 mmol) was added. The reaction was stirred a further 4 h then was diluted with water (30 mL) and extracted with DCM (50 mL×3). The combined organic layers were washed with brine, dried over Na₂SO₄, filtered and concentrated. The residue was purified prep-HPLC to afford (6-(benzo[d]thiazol-7-yl)-8-(5-(3,4-dichlorobenzyl)-1,3,4-oxadiazol-2-yl)-2,6-diazaspiro[3.4]octan-2-yl)((S)-2,2-dimethylcyclopropyl)methanone (100 mg, 50%) as a white solid. LCMS m/z=568.3 [M+H]⁺; ¹H NMR (400 MHz, Methanol-d₄) δ 9.16 (s, 1H), 7.54-7.48 (m, 2H), 7.46-7.37 (m, 2H), 7.24-7.19 (m, 1H), 6.71 (d, J=7.6 Hz, 1H), 4.50-3.91 (m, 12H), 1.47-1.32 (m, 1H), 1.21-1.04 (m, 6H), 1.02-1.00 (m, 1H), 0.81-0.73 (m, 1H).

### Synthesis of (8-(1-(3,4-dichlorobenzyl)-1H-1,2,3-triazol-4-yl)-2-((S)-2,2-dimethylcyclopropane-1-carbonyl)-2,6-diazaspiro[3.4]octan-6-yl)(thiazol-5-yl)methanone (I-51)

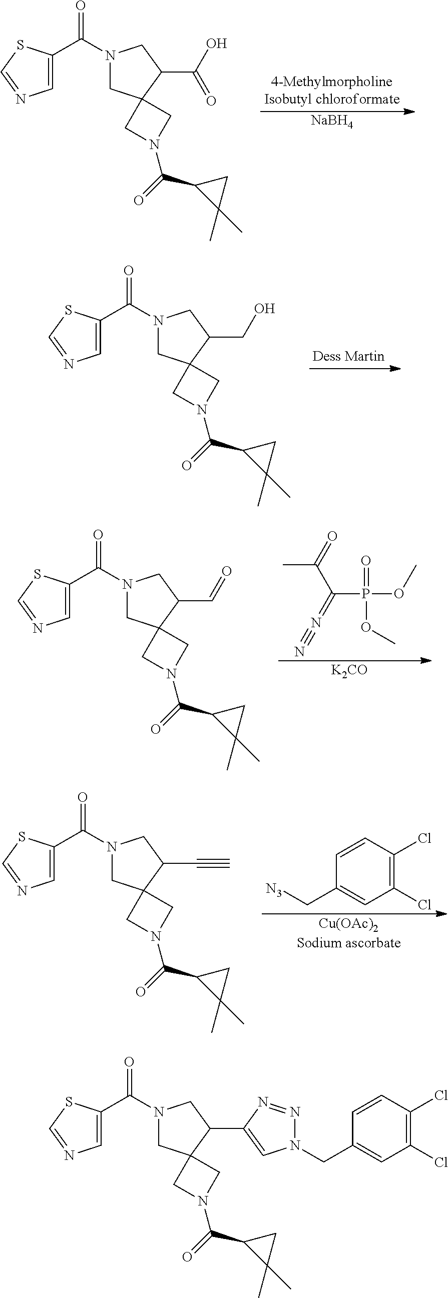

### Step 1: (2-((S)-2,2-dimethylcyclopropane-1-carbonyl)-8-(hydroxymethyl)-2,6-diazaspiro[3.4]octan-6-yl)(thiazol-5-yl)methanone

To a solution of 2-((S)-2,2-dimethylcyclopropane-1-carbonyl)-6-(thiazole-5-carbonyl)-2,6-diazaspiro[3.4]octane-8-carboxylic acid (500 mg, 1.38 mmol) in THF (5 mL) at 0° C. was added 4-methylmorpholine (181 mg, 1.79 mmol) and isobutyl chloroformate (263 mg, 1.93 mmol). The reaction was stirred for 30 min then a solution of NaBH₄ (154 mg, 4.13 mmol) in water (5 mL) was added. The reaction was stirred for another 0.5 h at 0° C. then the solvent removed under reduced pressure. The residue obtained was purified by column chromatography on silica gel (eluent: DCM/MeOH=40/1, v/v) to afford (2-((S)-2,2-dimethylcyclopropane-1-carbonyl)-8-(hydroxymethyl)-2,6-diazaspiro[3.4]octan-6-yl)(thiazol-5-yl)methanone (320 mg, 66%) as a white solid. LCMS m/z=350.1 [M+H]⁺; ¹H NMR (400 MHz, DMSO-d₆) δ 9.24 (s, 1H), 8.40-8.32 (m, 1H), 4.81 (s, 1H), 4.25-3.46 (m, 10H), 2.47-2.29 (m, 1H), 1.41-1.32 (m, 1H), 1.15-1.02 (m, 6H), 0.90-0.83 (m, 1H), 0.72-0.61 (m, 1H).

### Step 2: 2-((S)-2,2-dimethylcyclopropane-1-carbonyl)-6-(thiazole-5-carbonyl)-2,6-diazaspiro[3.4]octane-8-carbaldehyde

To a solution of (2-((S)-2,2-dimethylcyclopropane-1-carbonyl)-8-(hydroxymethyl)-2,6-diazaspiro[3.4]octan-6-yl)(thiazol-5-yl)methanone (20 mg, 0.057 mmol) in DCM (2 mL) at room temperature was added Dess-Martin reagent (122 mg, 0.28 mmol). The reaction mixture was stirred at room temperature for 2 h then filtered through Celite. The filtrate was diluted with DCM (20 mL) and the organic layer washed with aq. Na₂S₂O₃ (30 mL), aq. NaHCO₃ (30 mL) and brine, dried over Na₂SO₄ and concentrated to afford 2-((S)-2,2-dimethylcyclopropane-1-carbonyl)-6-(thiazole-5-carbonyl)-2,6-diazaspiro[3.4]octane-8-carbaldehyde (15 mg, 75%) as yellow solid. LCMS m/z=348.0 [M+H]⁺; ¹H NMR (400 MHz, DMSO-d₆) δ 9.83-9.73 (m, 1H), 9.30-9.21 (m, 1H), 8.43-8.35 (m, 1H), 4.28-3.86 (m, 6H), 3.69-3.46 (m, 2H), 1.91 (s, 1H), 1.40-1.33 (m, 1H), 1.14-1.04 (m, 6H), 0.88-0.85 (m, 1H), 0.68 (s, 1H).

### Step 3: (2-((S)-2,2-dimethylcyclopropane-1-carbonyl)-8-ethynyl-2,6-diazaspiro[3.4]octan-6-yl)(thiazol-5-yl)methanone

To a solution of 2-((S)-2,2-dimethylcyclopropane-1-carbonyl)-6-(thiazole-5-carbonyl)-2,6-diazaspiro[3.4]octane-8-carbaldehyde (10 mg, 0.028 mol) in MeOH (0.5 mL) at room temperature was added dimethyl (1-diazo-2-oxopropyl)phosphonate (7 mg, 0.034 mmol). The reaction mixture was stirred at room temperature overnight then diluted with water (10 mL) and extracted with EtOAc (20 mL×2). The combined organic layers were washed with brine, dried over Na₂SO₄, filtered and concentrated to afford (2-((S)-2,2-dimethylcyclopropane-1-carbonyl)-8-ethynyl-2,6-diazaspiro[3.4]octan-6-yl)(thiazol-5-yl)methanone (6 mg, 60%) as yellow solid. LCMS m/z=344.1 [M+H]⁺; ¹H NMR (400 MHz, DMSO-d₆) δ 9.26 (s, 1H), 8.44-8.34 (m, 1H), 4.40-4.22 (m, 1H), 4.19-4.02 (m, 3H), 3.91-3.70 (m, 4H), 3.27-3.22 (m, 1H), 2.04-1.94 (m, 1H), 1.40-1.36 (m, 1H), 1.14-1.10 (m, 3H), 1.07-1.04 (m, 3H), 0.87 (s, 1H), 0.73-0.63 (m, 1H).

### Step 4: (8-(1-(3,4-dichlorobenzyl)-1H-1,2,3-triazol-4-yl)-2-((S)-2,2-dimethylcyclopropane-1-carbonyl)-2,6-diazaspiro[3.4]octan-6-yl)(thiazol-5-yl)methanone

To a solution of (2-((S)-2,2-dimethylcyclopropane-1-carbonyl)-8-ethynyl-2,6-diazaspiro[3.4]octan-6-yl)(thiazol-5-yl)methanone (60 mg, 0.17 mmol) in a mixture of water and tBuOH (1/1 mL) at room temperature was added 4-(azidomethyl)-1,2-dichlorobenzene (71 mg, 0.35 mmol), Cu(OAc)₂ (7 mg, 0.04 mmol) and Na-ascorbate (35 mg, 0.17 mmol). The mixture was heated at 80° C. for 4 h then was cooled and concentrated under reduced pressure. The residue obtained was purified by prep-HPLC to afford (8-(1-(3,4-dichlorobenzyl)-1H-1,2,3-triazol-4-yl)-2-((S)-2,2-dimethylcyclopropane-1-carbonyl)-2,6-diazaspiro[3.4]octan-6-yl)(thiazol-5-yl)methanone (6 mg, 6%) as white solid. LCMS m/z=545.1 [M+H]⁺; ¹H NMR (400 MHz, Chloroform-d) δ 8.92 (d, J=6.8 Hz, 1H), 8.28 (s, 1H), 7.49-7.35 (m, 3H), 7.16-7.03 (m, 1H), 5.58-5.39 (m, 2H), 4.39-3.63 (m, 9H), 1.24-0.97 (m, 8H), 0.78-0.64 (m, 1H).

### Synthesis of (8-(5-(3,4-dichlorobenzyl)-1,3,4-oxadiazol-2-yl)-2-(pyrimidin-2-yl)-2,6-diazaspiro[3.4]octan-6-yl)(thiazol-5-yl)methanone (I-49)

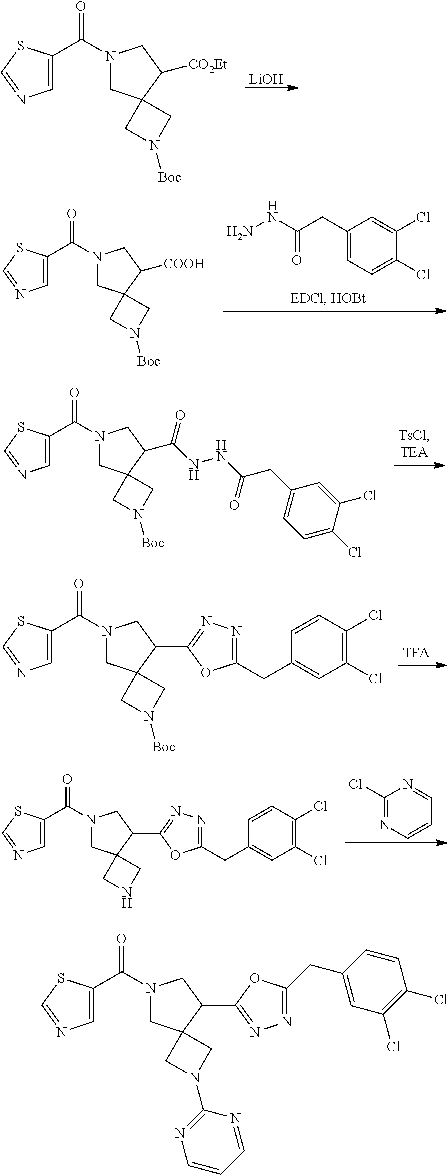

### Step 1: 2-(tert-butoxycarbonyl)-6-(thiazole-5-carbonyl)-2,6-diazaspiro[3.4]octane-8-carboxylic acid

To a solution of 2-(tert-butyl) 8-ethyl 6-(thiazole-5-carbonyl)-2,6-diazaspiro[3.4]octane-2,8-dicarboxylate (4.05 g, 10.2 mmol) in a mixture of THF and water (40 mL/10 mL) was added LiOH (0.52 g, 12.3 mmol). The reaction was stirred at room temperature for 2 h, then diluted with water (40 mL) and extracted with ether (50 mL). The aqueous layer was collected and acidified to pH˜2 with 1M HCl then extracted with EtOAc (100 mL×2). The combined organic layers were washed with brine, dried over Na₂SO₄, filtered and concentrated to afford 2-(tert-butoxycarbonyl)-6-(thiazole-5-carbonyl)-2,6-diazaspiro[3.4]octane-8-carboxylic acid (3.2 g, 100%) as a white solid which was used directly in the next step. LCMS m/z=367.9 [M+H]⁺; ¹H NMR (400 MHz, Chloroform-d) δ 8.94 (s, 1H), 8.26 (s, 1H), 4.22-3.79 (m, 9H), 1.43 (s, 10H).

### Step 2: tert-butyl 8-(2-(2-(3,4-dichlorophenyl)acetyl)hydrazine-1-carbonyl)-6-(thiazole-5-carbonyl)-2,6-diazaspiro[3.4]octane-2-carboxylate

To a solution of 2-(3,4-dichlorophenyl)acetohydrazide (2.1 g, 9.6 mmol) in DMF (50 mL) was added 2-(tert-butoxycarbonyl)-6-(thiazole-5-carbonyl)-2,6-diazaspiro[3.4]octane-8-carboxylic acid (3.2 g, 8.7 mmol), EDCI (2.2 g, 11.5 mmol), HOBt (1.53 g, 11.3 mmol) and DIPEA (3.38 g, 26.1 mmol). The resulting mixture was stirred at room temperature for 5 h then diluted with water (100 mL) and extracted with EtOAc (100 mL×3). The combined organic layers were washed with water and brine, dried over Na₂SO₄, filtered and concentrated. The residue was purified by column chromatography on silica gel (eluent: 5% MeOH in DCM) to afford ethyl tert-butyl 8-(2-(2-(3,4-dichlorophenyl)acetyl)hydrazine-1-carbonyl)-6-(thiazole-5-carbonyl)-2,6-diazaspiro[3.4]octane-2-carboxylate (2.45 g, 49%) as a white solid. LCMS m/z=567.9 [M+H]⁺; ¹H NMR (400 MHz, Methanol-d4) δ 9.15 (s, 1H), 8.36 (d, J=12.8 Hz, 1H), 7.52 (s, 1H), 7.46 (d, J=8.3 Hz, 1H), 7.27 (s, 1H), 4.29-3.78 (m, 9H), 3.57 (s, 2H), 3.29-3.19 (m, 1H), 1.42 (s, 9H).

### Step 3: tert-butyl 8-(5-(3,4-dichlorobenzyl)-1,3,4-oxadiazol-2-yl)-6-(thiazole-5-carbonyl)-2,6-diazaspiro[3.4]octane-2-carboxylate

To a solution of 8-(2-(2-(3,4-dichlorophenyl)acetyl)hydrazine-1-carbonyl)-6-(thiazole-5-carbonyl)-2,6-diazaspiro[3.4]octane-2-carboxylate (2.45 g, 4.3 mmol) in DCM (30 mL) was added TsCl (1.64 g, 8.6 mmol) and TEA (0.87 g, 8.6 mmol). The reaction was stirred at room temperature for 14 h, then diluted with water (40 mL) and extracted with DCM (50 mL×2). The combined organic layers were washed with aqueous NH₄Cl and brine, dried over Na₂SO₄, filtered and concentrated. The residue was purified by RP-column (MeCN:water=50%) to afford tert-butyl 8-(5-(3,4-dichlorobenzyl)-1,3,4-oxadiazol-2-yl)-6-(thiazole-5-carbonyl)-2,6-diazaspiro[3.4]octane-2-carboxylate (1.56 g, 65%) as a white solid. LCMS m/z=549.9 [M+H]⁺.

### Step 4: (8-(5-(3,4-dichlorobenzyl)-1,3,4-oxadiazol-2-yl)-2,6-diazaspiro[3.4]octan-6-yl)(thiazol-5-yl)methanone

To a solution of tert-butyl 8-(5-(3,4-dichlorobenzyl)-1,3,4-oxadiazol-2-yl)-6-(thiazole-5-carbonyl)-2,6-diazaspiro[3.4]octane-2-carboxylate (1.56 g, 2.83 mmol) in DCM (15 mL) was added TFA (6 mL). The reaction mixture was stirred at room temperature for 3 h then the solvent was removed under reduced pressure. The residue was diluted with water (20 mL) and extracted with MTBE (30 mL×2). The aqueous was collected, the pH adjusted to 10 with 10 M NaOH then the basic solution was extracted with EtOAc (80 mL×2). The combined organic layers were dried over Na₂SO₄ and concentrated to afford (8-(5-(3,4-dichlorobenzyl)-1,3,4-oxadiazol-2-yl)-2,6-diazaspiro[3.4]octan-6-yl)(thiazol-5-yl)methanone (1.09 g, 85%). LCMS m/z=449.9 [M+H]⁺; ¹H NMR (400 MHz, Methanol-d4) δ 9.16 (d, J=9.5 Hz, 1H), 8.36 (d, J=20.0 Hz, 1H), 7.58-7.52 (m, 1H), 7.49 (d, J=8.3 Hz, 1H), 7.28 (d, J=7.1 Hz, 1H), 4.32-3.51 (m, 12H).

### Step 5: (8-(5-(3,4-dichlorobenzyl)-1,3,4-oxadiazol-2-yl)-2-(pyrimidin-2-yl)-2,6-diazaspiro[3.4]octan-6-yl)(thiazol-5-yl)methanone

To a solution of (8-(5-(3,4-dichlorobenzyl)-1,3,4-oxadiazol-2-yl)-2,6-diazaspiro[3.4]octan-6-yl)(thiazol-5-yl)methanone (100 mg, 0.22 mmol) and 2-chloropyrimidine (25 mg, 0.22 mmol) in CH₃CN (1 mL) was added Na₂CO₃ (71 mg, 0.67 mmol) and the reaction heated at 70° C. for 2 h. The solvent was removed under reduced pressure and the residue obtained purified by RP-column to afford (8-(5-(3,4-dichlorobenzyl)-1,3,4-oxadiazol-2-yl)-2-(pyrimidin-2-yl)-2,6-diazaspiro[3.4]octan-6-yl)(thiazol-5-yl)methanone (17.5 mg, 15%) as a white solid. LCMS m/z=528.1 ¹H NMR (400 MHz, DMSO-d₆) δ 9.26 (s, 1H), 8.43-8.30 (m, 3H), 7.63-7.56 (m, 1H), 7.51-7.46 (m, 1H), 7.28-7.19 (m, 1H), 6.72-6.66 (m, 1H), 4.28-4.07 (m, 7H), 4.01-3.87 (m, 4H).

Table 14: The compounds listed in Table 14 were synthesized from (8-(5-(3,4-dichlorobenzyl)-1,3,4-oxadiazol-2-yl)-2,6-diazaspiro[3.4]octan-6-yl)(thiazol-5-yl)methanone according to the procedures outlined for I-49 using the appropriate commercially available reagents and/or intermediates described elsewhere.

TABLE 14

| Example | | | |
|---|---|---|---|
| Number | Compound | ¹HNMR | LCMS |
| I-41 | 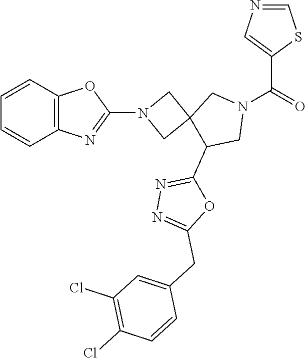 [SMILES: O=C(c1cncs1)N1CC(c2nnc(Cc3ccc(Cl)c(Cl)c3)o2)C2(C1)CN(c1nc3ccccc3o1)C2] | ¹H NMR (400 MHz, DMSO-d₆) δ 9.27 (s, 1H), 8.39 (d, J = 9.8 Hz, 1H), 7.62-7.56 (m, 1H), 7.48 (dd, J = 8.2, 5.6 Hz, 1H), 7.41 (d, J = 8.0 Hz, 1H), 7.35-7.29 (m, 1H), 7.24 (t, J = 10.0 Hz, 1H), 7.17 (t, J = 7.8 Hz, 1H), 7.05 (t, J = 8.0 Hz, 1H), 4.36-4.29 (m, 2H), 4.24 (dt, J = 16.8, 8.2 Hz, 5H), 4.17-4.09 (m, 2H), 3.95 (d, J = 6.8 Hz, 2H). | m/z = 567.1 [M + H]⁺ |
| I-60 | 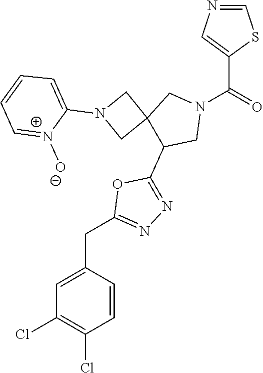 [SMILES: O=C(c1cncs1)N1CC(c2nnc(Cc3ccc(Cl)c(Cl)c3)o2)C2(C1)CN(c1cccc[n+]1[O-])C2] | ¹H NMR (400 MHz, Methanol-d₄) δ 9.17 (s, 1H), 8.39 (d, J = 3.8 Hz, 1H), 7.97 (d, J = 6.6 Hz, 1H), 7.51 (d, J = 7.8 Hz, 1H), 7.47-7.35 (m, 2H), 7.25-7.18 (m, 1H), 6.86 (s, 1H), 6.62 (d, J = 8.0 Hz, 1H), 4.58-4.38 (m, 2H), 4.36-4.20 (m, 6H), 4.16-3.99 (m, 3H). | m/z = 543.1 [M + H]⁺ |
| I-94 | 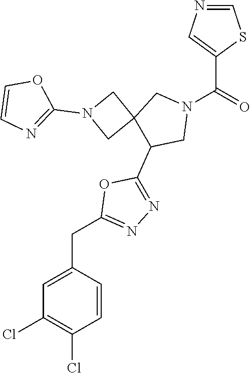 [SMILES: O=C(c1cncs1)N1CC(c2nnc(Cc3ccc(Cl)c(Cl)c3)o2)C2(C1)CN(c1ncco1)C2] | ¹H NMR (400 MHz, Methanol-d4) δ 9.16 (s, 1H), 8.37 (s, 1H), 7.50 (d, J = 6.8 Hz, 1H), 7.42- 7.38 (m, 2H), 7.19 (t, J = 7.6 Hz, 1H), 6.81 (d, J = 5.2 Hz, 1H), 4.28- 4.21 (m, 6H), 4.10-4.03 (m, 5H). | m/z = 517.2 [M + H]⁺ |
| I-99 | 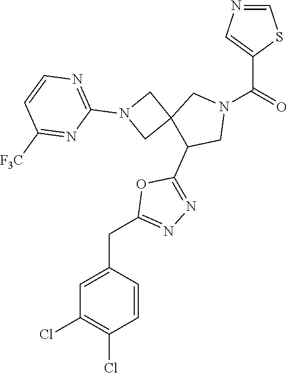 [SMILES: O=C(c1cncs1)N1CC(c2nnc(Cc3ccc(Cl)c(Cl)c3)o2)C2(C1)CN(c1nccc(C(F)(F)F)n1)C2] | ¹H NMR (400 MHz, DMSO-d₆) δ 9.26 (s, 1H), 8.66 (t, J = 4.0 Hz, 1H), 8.38 (d, J = 8.0 Hz, 1H), 7.63-7.55 (m, 1H), 7.49 (d, J = 8.0 Hz, 1H), 7.26 (d, J = 8.0 Hz, 1H), 7.09 (t, J = 4.0 Hz, 1H), 4.35-4.06 (m, 8H), 4.04- 3.86 (m, 3H). | m/z = 596.1 [M + H]⁺ |
| I-47 | 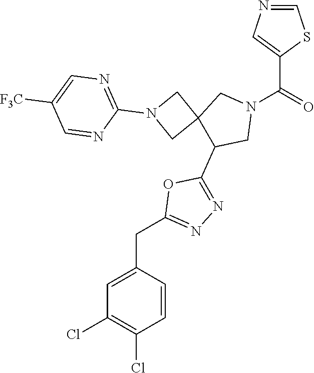 [SMILES: O=C(c1cncs1)N1CC(c2nnc(Cc3ccc(Cl)c(Cl)c3)o2)C2(C1)CN(c1ncc(C(F)(F)F)cn1)C2] | ¹H NMR (400 MHz, DMSO-d₆) δ 9.26 (s, 1H), 8.67 (s, 2H), 8.38 (s, 1H), 7.60 (s, 1H), 7.48 (s, 1H), 7.25 (s, 1H), 4.38- 3.81 (m, 12H) | m/z = 596.10 [M + H]⁺ |

### Synthesis of (8-(5-(3,4-dichlorobenzyl)-1,3,4-oxadiazol-2-yl)-2-(pyridin-2-yl)-2,6-diazaspiro[3.4]octan-6-yl)(thiazol-5-yl)methanone (I-197)

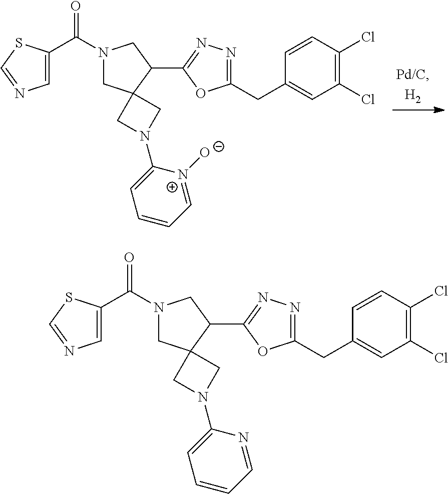

### Step 1: (8-(5-(3,4-dichlorobenzyl)-1,3,4-oxadiazol-2-yl)-2-(pyridin-2-yl)-2,6-diazaspiro[3.4]octan-6-yl)(thiazol-5-yl)methanone

To a solution of 2-(8-(5-(3,4-dichlorobenzyl)-1,3,4-oxadiazol-2-yl)-6-(thiazole-5-carbonyl)-2,6-diazaspiro[3.4]octan-2-yl)pyridine 1-oxide (50 mg, 0.09 mmol) in EtOH (3 mL) was added 10% Pd/C (25 mg). The reaction mixture was heated at 78° C. under a H₂ atmosphere overnight, 50% was observed. The catalyst was removed by filtration through celite and the filtrate concentrated. The residue obtained was redissolved in EtOH (3 mL) and another batch of 10% Pd/C (25 mg) was added. The reaction was heated at 78° C. under H₂ atmosphere overnight. The catalyst was removed by filtration through celite and the filtrate concentrated. The residue was purified by prep-TLC to afford (8-(5-(3,4-dichlorobenzyl)-1,3,4-oxadiazol-2-yl)-2-(pyridin-2-yl)-2,6-diazaspiro[3.4]octan-6-yl)(thiazol-5-yl)methanone (12 mg, 25%) as a white solid. LCMS m/z=527.1 [M+H]⁺; ¹H NMR (400 MHz, Methanol-d₄) δ 9.17 (s, 1H), 8.39 (d, J=3.8 Hz, 1H), 7.97 (d, J=6.6 Hz, 1H), 7.51 (d, J=7.8 Hz, 1H), 7.47-7.35 (m, 2H), 7.25-7.18 (m, 1H), 6.86 (s, 1H), 6.62 (d, J=8.0 Hz, 1H), 4.58-4.38 (m, 2H), 4.36-4.20 (m, 6H), 4.16-3.99 (m, 3H).

### Synthesis of (8-(5-((3,4-dichlorophenyl)difluoromethyl)-1,3,4-oxadiazol-2-yl)-2-(pyrimidin-2-yl)-2,6-diazaspiro[3.4]octan-6-yl)(thiazol-5-yl)methanone (I-59)

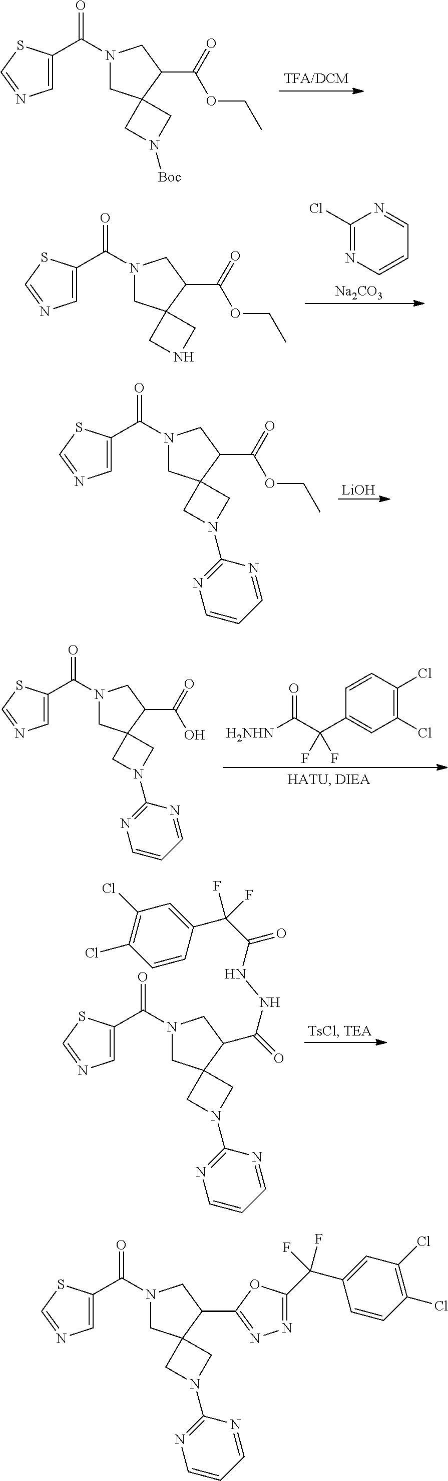

### Step 1: ethyl 6-(thiazole-5-carbonyl)-2,6-diazaspiro[3.4]octane-8-carboxylate

To a solution of 2-(tert-butyl) 8-ethyl 6-(thiazole-5-carbonyl)-2,6-diazaspiro[3.4]octane-2,8-dicarboxylate (500 mg, 1.27 mmol) in DCM (10 mL) was added TFA (4 mL) and the reaction stirred at room temperature for 2 h. The solvent was removed under reduced pressure to afford ethyl 6-(thiazole-5-carbonyl)-2,6-diazaspiro[3.4]octane-8-carboxylate (373 mg, TFA salt) which was used directly in the next step. LCMS m/z=295.1 [M+H].

### Step 2: ethyl 2-(pyrimidin-2-yl)-6-(thiazole-5-carbonyl)-2,6-diazaspiro[3.4]octane-8-carboxylate

To a solution of ethyl 6-(thiazole-5-carbonyl)-2,6-diazaspiro[3.4]octane-8-carboxylate (373 mg, 1.26 mmol) in MCN (5 mL) was added Na₂CO₃ (402 mg, 3.79 mmol). The reaction was stirred at room temperature for 30 minutes then 2-chloropyrimidine (174 mg, 1.52 mmol) was added. The reaction was heated at 70° C. for 2 h then diluted with water (20 mL) and extracted with EtOAc (50 mL×2). The combined organic layers were washed with brine, dried over Na₂SO₄, filtered and concentrated. The residue was purified by column chromatography on silica gel (eluent: Pet. Ether:EtOAc=2:1) to afford ethyl 2-(pyrimidin-2-yl)-6-(thiazole-5-carbonyl)-2,6-diazaspiro[3.4]octane-8-carboxylate (300 mg, 64%) as a white solid. LCMS m/z=373.12 [M+H]⁺.

### Step 3: 2-(pyrimidin-2-yl)-6-(thiazole-5-carbonyl)-2,6-diazaspiro[3.4]octane-8-carboxylic acid

To a solution of ethyl 2-(pyrimidin-2-yl)-6-(thiazole-5-carbonyl)-2,6-diazaspiro[3.4]octane-8-carboxylate (450 mg, 1.21 mmol) in a mixture of THF, MeOH and water (4 mL/1 mL/1 mL) was added LiOH (72 mg, 3.02 mmol) and the reaction stirred at room temperature for 2 h. The mixture was diluted with water (10 mL) and extracted with EtOAc (30 mL). The aqueous layer was collected and acidified to pH˜2 with 1M HCl and extracted with EtOAc (80 mL×2). The combined organic layers were washed with brine, dried over Na₂SO₄, filtered and concentrated to afford 2-(pyrimidin-2-yl)-6-(thiazole-5-carbonyl)-2,6-diazaspiro[3.4]octane-8-carboxylic acid (382 mg, 92%) as a white solid which was used directly in the next step. LCMS m/z=345.1 [M+H]⁺; ¹H NMR (DMSO, 400 MHz) δ 9.25 (1H, s), 8.32-8.44 (3H, m), 6.69 (1H, s), 3.95-4.19 (5H, m), 3.85 (1H, s), 3.75 (1H, d, J=6.6 Hz), 3.38 (2H, s).

### Step 4: N′-(2-(3,4-dichlorophenyl)-2,2-difluoroacetyl)-2-(pyrimidin-2-yl)-6-(thiazole-5-carbonyl)-2,6-diazaspiro[3.4]octane-8-carbohydrazide

To a solution of 2-(pyrimidin-2-yl)-6-(thiazole-5-carbonyl)-2,6-diazaspiro[3.4]octane-8-carboxylic acid (190 mg, 0.55 mmol) in DCM (5 mL) was added HATU (230 mg, 0.61 mmol) and DIEA (214 mg, 1.65 mmol) and the reaction stirred for 30 min. N′-(2-(3,4-dichlorophenyl)-2,2-difluoroacetyl)-2-(pyrimidin-2-yl)-6-(thiazole-5-carbonyl)-2,6-diazaspiro[3.4]octane-8-carbohydrazide (154 mg, 0.61 mol) was added and the reaction stirred a further 4 h. The mixture was diluted with water (30 mL) and extracted with EtOAc (100 mL×2). The combined organic layers were washed with brine, dried over Na₂SO₄, filtered and concentrated. The residue obtained was purified by prep-TLC (eluent: Pet. DCM:MeOH=10:1) to afford N′-(2-(3,4-dichlorophenyl)-2,2-difluoroacetyl)-2-(pyrimidin-2-yl)-6-(thiazole-5-carbonyl)-2,6-diazaspiro[3.4]octane-8-carbohydrazide (209 mg, 65%) as a white solid. LCMS m/z=581.1 [M+H]⁺; ¹H NMR (DMSO, 400 MHz) δ 11.14 (1H, s), 10.45 (1H, s), 9.25 (1H, s), 8.33-8.41 (3H, m), 7.87 (2H, d, J=9.0 Hz), 7.63 (1H, d, J=8.0 Hz), 6.69 (1H, d, J=4.4 Hz), 4.25 (2H, d, J=9.2 Hz), 3.97-4.10 (4H, m), 3.77 (2H, dd, J=18.2, 10.7 Hz), 2.00 (1H, d, J=7.6 Hz).

### Step 5: (8-(5-((3,4-dichlorophenyl)difluoromethyl)-1,3,4-oxadiazol-2-yl)-2-(pyrimidin-2-yl)-2,6-diazaspiro[3.4]octan-6-yl)(thiazol-5-yl)methanone

To a solution of N′-(2-(3,4-dichlorophenyl)-2,2-difluoroacetyl)-2-(pyrimidin-2-yl)-6-(thiazole-5-carbonyl)-2,6-diazaspiro[3.4]octane-8-carbohydrazide (50 mg, 0.1 mmol) in DCM (2 mL) was added TsCl (49 mg, 0.3 mmol) and TEA (44 mg, 0.4 mmol). The reaction mixture was stirred at room temperature for 2 h then diluted with water (20 mL) and extracted with DCM (50 mL×2). The combined organic layers were washed with brine, dried over Na₂SO₄, filtered and concentrated. The residue obtained was purified by prep-TLC (eluent: Pet. DCM:MeOH=15:1) to afford (8-(5-((3,4-dichlorophenyl)difluoromethyl)-1,3,4-oxadiazol-2-yl)-2-(pyrimidin-2-yl)-2,6-diazaspiro[3.4]octan-6-yl)(thiazol-5-yl)methanone (41 mg, 85%) as a white solid. LCMS m/z=563.1 [M+H]⁺; ¹H NMR (DMSO, 400 MHz) δ 9.27 (1H, s), 8.33 (3H, d, J=4.4 Hz), 7.96 (1H, d, J=8.2 Hz), 7.81 (1H, s), 7.66 (1H, d, J=8.2 Hz), 6.70 (1H, d, J=4.4 Hz), 4.22 (5H, d, J=38.4 Hz), 3.99 (4H, d, J=23.2 Hz).

### Synthesis of (1-benzyl-1H-pyrazol-4-yl)(8-(5-((4-bromophenyl)amino)-1,3,4-oxadiazol-2-yl)-2-((S)-2,2-dimethylcyclopropane-1-carbonyl)-2,6-diazaspiro[3.4]octan-6-yl)methanone (I-5)

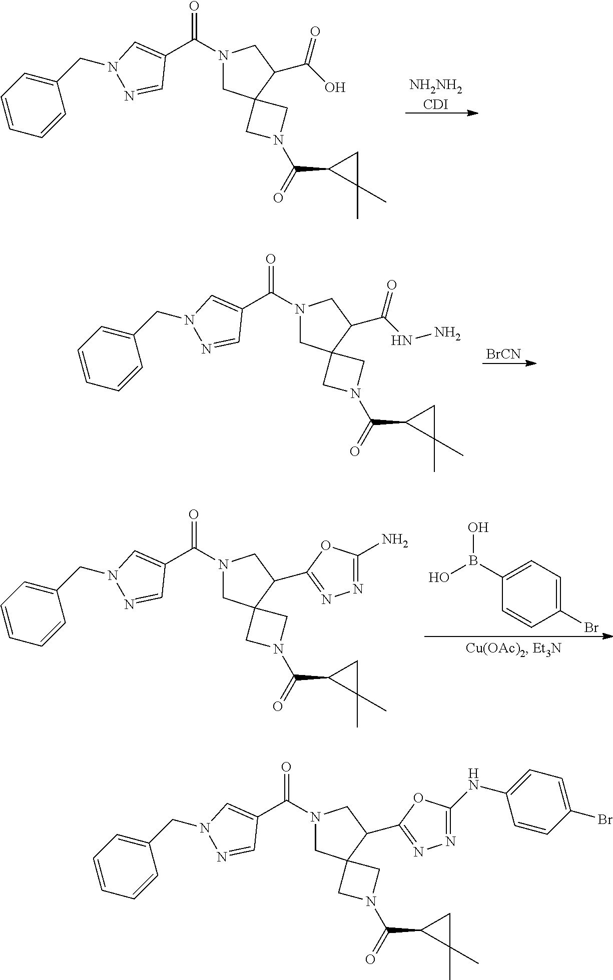

### Step 1: 6-(1-benzyl-1H-pyrazole-4-carbonyl)-2-((S)-2,2-dimethylcyclopropane-1-carbonyl)-2,6-diazaspiro[3.4]octane-8-carbohydrazide

To a solution of 6-(1-benzyl-1H-pyrazole-4-carbonyl)-2-((S)-2,2-dimethylcyclopropane-1-carbonyl)-2,6-diazaspiro[3.4]octane-8-carboxylic acid (200 mg, 0.458 mmol) in THF (2 mL) at 0° C. was added CDI (90 mg, 0.458 mmol) and NH₂NH₂ (70 mg, 1.374 mmol). The reaction was stirred at room temperature overnight then diluted with water (20 mL) and extracted with a mixture of DCM and MeOH (5/1, 80 mL×2). The combined organic layers were washed with brine, dried over Na₂SO₄, filtered and concentrated to afford 6-(1-benzyl-1H-pyrazole-4-carbonyl)-2-((S)-2,2-dimethylcyclopropane-1-carbonyl)-2,6-diazaspiro[3.4]octane-8-carbohydrazide (190 mg, 92% yield) as a white solid. LCMS m/z=451.2 [M+H]⁺.

### Step 2: (8-(5-amino-1,3,4-oxadiazol-2-yl)-2-((S)-2,2-dimethylcyclopropane-1-carbonyl)-2,6-diazaspiro[3.4]octan-6-yl)(1-benzyl-1H-pyrazol-4-yl)methanone

To a solution of 6-(1-benzyl-1H-pyrazole-4-carbonyl)-2-((S)-2,2-dimethylcyclopropane-1-carbonyl)-2,6-diazaspiro[3.4]octane-8-carbohydrazide (190 mg, 0.422 mmol) in a mixture of dioxane and water (3:1, 2 mL) was added BrCN (51 mg, 0.422 mmol) and NaHCO₃ (35 mg, 0.422 mmol) and the reaction stirred at room temperature overnight. The reaction was diluted with water (20 mL) and extracted with a mixture of DCM and MeOH (5/1, 80 mL×2). The combined organic layers were washed with brine, dried over Na₂SO₄, filtered and concentrated to afford (8-(5-amino-1,3,4-oxadiazol-2-yl)-2-((S)-2,2-dimethylcyclopropane-1-carbonyl)-2,6-diazaspiro[3.4]octan-6-yl)(1-benzyl-1H-pyrazol-4-yl)methanone (60 mg, 30%) as a white solid. LCMS m/z=476.2 [M+H]⁺.

### Step 3: (1-benzyl-1H-pyrazol-4-yl)(8-(5-((4-bromophenyl)amino)-1,3,4-oxadiazol-2-yl)-2-((S)-2,2-dimethylcyclopropane-1-carbonyl)-2,6-diazaspiro[3.4]octan-6-yl)methanone

To a solution of (8-(5-amino-1,3,4-oxadiazol-2-yl)-2-((S)-2,2-dimethylcyclopropane-1-carbonyl)-2,6-diazaspiro[3.4]octan-6-yl)(1-benzyl-1H-pyrazol-4-yl)methanone (40 mg, 0.084 mmol) in DCM (2 mL) was added (4-bromophenyl)boronic acid (51 mg, 0.252 mmol), Cu(OAc)₂ (45 mg, 0.252 mmol) and Et₃N (42 mg, 0.420 mmol). The resulting mixture was stirred under an oxygen atmosphere overnight then was diluted with water (15 mL) and extracted with EtOAc (50 mL×2). The combined organic layers were washed with brine, dried over Na₂SO₄, filtered and concentrated. The residue was purified by prep-HPLC to afford (1-benzyl-1H-pyrazol-4-yl)(8-(5-((4-bromophenyl)amino)-1,3,4-oxadiazol-2-yl)-2-((S)-2,2-dimethylcyclopropane-1-carbonyl)-2,6-diazaspiro[3.4]octan-6-yl)methanone (9 mg, 17%) as a white solid. LCMS m/z=632.2 [M+H]⁺; ¹H NMR (400 MHz, CD₃OD) δ 8.26-8.21 (m 1H), 7.97-7.93 (m, 1H), 7.50-7.25 (m, 9H), 5.39 (s, 2H), 4.50-3.87 (m, 9H), 1.45-1.33 (m, 2H), 1.16-0.98 (m, 5H), 0.93-0.89 (m, 1H), 0.80-0.69 (m, 1H).

### Synthesis of (1-benzyl-1H-pyrazol-4-yl)(8-(5-(1-(3,5-dimethyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)methanone (I-21)

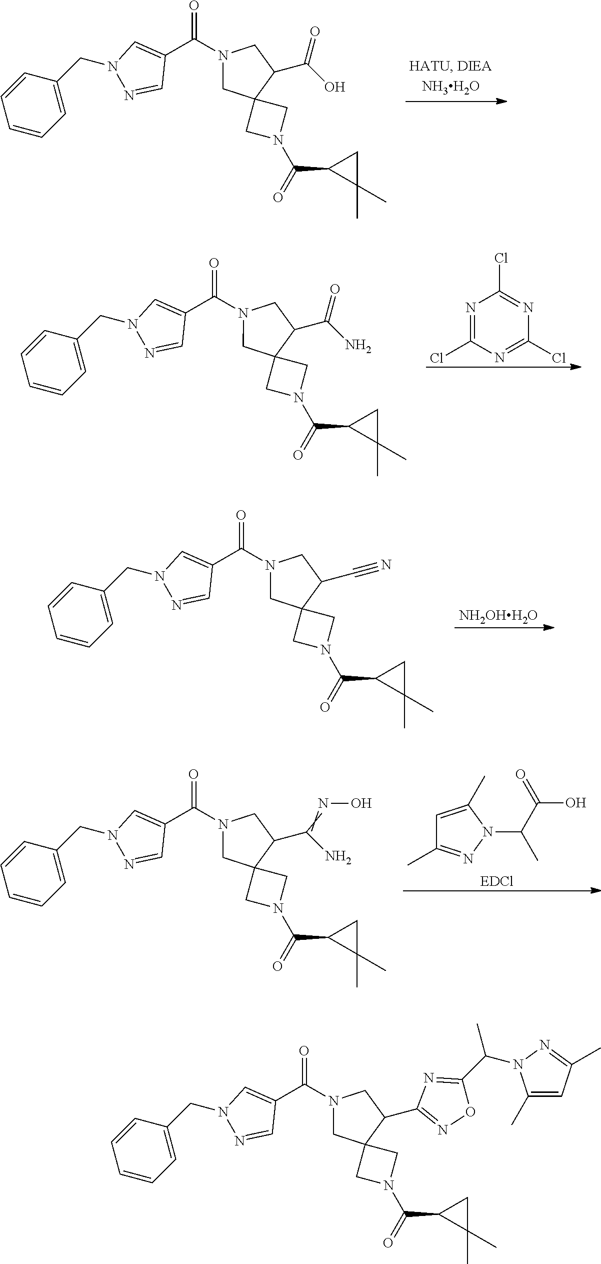

### Step 1: 6-(1-benzyl-1H-pyrazole-4-carbonyl)-2-((S)-2,2-dimethylcyclopropane-1-carbonyl)-2,6-diazaspiro[3.4]octane-8-carboxamide

To a solution of 6-(1-benzyl-1H-pyrazole-4-carbonyl)-2-((S)-2,2-dimethylcyclopropane-1-carbonyl)-2,6-diazaspiro[3.4]octane-8-carboxylic acid (1.0 g, 2.29 mmol) in DMF (15 mL) was added HATU (1.31 g, 3.44 mmol) and DIPEA (888 mg, 6.87 mmol) and the reaction stirred for 30 min. NH₃·H₂O (100 mL) was added and the reaction stirred a further 2 h. The solvent was removed under reduced pressure and the residue obtained purified by RP-column (24% MeCN in water) to afford 6-(1-benzyl-1H-pyrazole-4-carbonyl)-2-((S)-2,2-dimethylcyclopropane-1-carbonyl)-2,6-diazaspiro[3.4]octane-8-carboxamide (780 mg, 78%) as a white solid. LCMS m/z=436.3 [M+H]⁺; ¹H NMR (400 MHz, Methanol-d4) δ 8.25-8.18 (m, 1H), 7.92 (d, J=9.8 Hz, 1H), 7.38-7.24 (m, 5H), 5.38 (s, 2H), 4.52-3.68 (m, 9H), 1.47-1.39 (m, 1H), 1.21-1.09 (m, 7H), 1.07-1.00 (m, 1H), 0.81-0.73 (m, 1H).

### Step 2: 6-(1-benzyl-1H-pyrazole-4-carbonyl)-2-((S)-2,2-dimethylcyclopropane-1-carbonyl)-2,6-diazaspiro[3.4]octane-8-carbonitrile

To a solution of 6-(1-benzyl-1H-pyrazole-4-carbonyl)-2-((S)-2,2-dimethylcyclopropane-1-carbonyl)-2,6-diazaspiro[3.4]octane-8-carboxamide (780 mg, 1.79 mmol) in DMF (12 mL) at 0° C. was added 2,4,6-trichloro-1,3,5-triazine (330 mg, 1.79 mmol). The reaction was stirred at 0° C. for 2 h then diluted with water and extracted with EtOAc (50 mL×3). The combined organic layers were washed with water and brine, dried over Na₂SO₄ and concentrated to give 6-(1-benzyl-1H-pyrazole-4-carbonyl)-2-((S)-2,2-dimethylcyclopropane-1-carbonyl)-2,6-diazaspiro[3.4]octane-8-carbonitrile (430 mg, 57%) as a yellow solid. LCMS m/z=418.2 [M+H]⁺; ¹H NMR (400 MHz, Methanol-d4) δ 8.23 (d, J=13.8 Hz, 1H), 7.93 (d, J=13.8 Hz, 1H), 7.40-7.25 (m, 5H), 5.39 (s, 2H), 4.60-3.64 (m, 9H), 1.52-1.40 (m, 1H), 1.24-1.11 (m, 6H), 1.10-1.03 (m, 1H), 0.86-0.76 (m, 1H).

### Step 3: 6-(1-benzyl-1H-pyrazole-4-carbonyl)-2-((S)-2,2-dimethylcyclopropane-1-carbonyl)-N′-hydroxy-2,6-diazaspiro[3.4]octane-8-carboximidamide

To a solution of 6-(1-benzyl-1H-pyrazole-4-carbonyl)-2-((S)-2,2-dimethylcyclopropane-1-carbonyl)-2,6-diazaspiro[3.4]octane-8-carbonitrile (430 mg, 1.03 mmol) in EtOH (10.0 mL) was added NH₂OH·H₂O (50% in water, 1.5 mL). The reaction was heated at 80° C. for 3 h then the solvent was removed under reduced pressure to afford 6-(1-benzyl-1H-pyrazole-4-carbonyl)-2-((S)-2,2-dimethylcyclopropane-1-carbonyl)-N′-hydroxy-2,6-diazaspiro[3.4]octane-8-carboximidamide (500 mg, quant.) as a white solid which was used without further purification. LCMS m/z=451.2 [M+H]⁺; ¹H NMR (400 MHz, Methanol-d4) δ 8.20 (d, J=12.4 Hz, 1H), 7.92 (d, J=7.6 Hz, 1H), 7.38-7.23 (m, 5H), 5.38 (s, 2H), 4.64-3.64 (m, 9H), 1.46-1.36 (m, 1H), 1.21-1.08 (m, 6H), 1.06-1.01 (m, 1H), 0.80-0.71 (m, 1H).

### Step 4: (1-benzyl-1H-pyrazol-4-yl)(8-(5-(1-(3,5-dimethyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)methanone

To a solution of 2-(3,5-dimethyl-1H-pyrazol-1-yl)propanoic acid (112 mg, 0.66 mmol) in a mixture of DMF and 1,4-dioxane (4 mL and 1 mL) was added ECDI (128 mg, 0.66 mmol) and 6-(1-benzyl-1H-pyrazole-4-carbonyl)-2-((S)-2,2-dimethylcyclopropane-1-carbonyl)-N′-hydroxy-2,6-diazaspiro[3.4]octane-8-carboximidamide (150 mg, 0.33 mmol). The reaction was heated at 60° C. for 6 h then the temperature raised to 100° C. and heating continued for 14 h. The solvent was removed under reduced pressure and the residue obtained was purified by column chromatography on silica gel (eluent: DCM:MeOH=50:1) to afford (1-benzyl-1H-pyrazol-4-yl)(8-(5-(1-(3,5-dimethyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)methanone (110 mg, 56%) as a white solid. LCMS m/z=583.4 [M+H]⁺; ¹H NMR (400 MHz, Methanol-d4) δ 8.26-8.17 (m, 1H), 7.96-7.87 (m, 1H), 7.44-7.16 (m, 5H), 5.93-5.75 (m, 2H), 5.39 (s, 2H), 4.61-3.79 (m, 9H), 2.38-2.24 (m, 3H), 2.13 (s, 3H), 1.98-1.82 (m, 3H), 1.47-1.26 (m, 2H), 1.22-1.16 (m, 2H), 1.16-0.99 (m, 5H), 0.83-0.68 (m, 1H). Chiral HPLC of the mixture afforded the two diastereomers: First eluting diastereomer (30 mg, I-2) as a white solid. LCMS m/z=583.3 [M+H]⁺; ¹H NMR (400 MHz, CD₃OD) δ 8.29-8.11 (m, 1H), 7.99-7.84 (m, 1H), 7.40-7.21 (m, 5H), 5.97-5.73 (m, 2H), 5.39 (s, 2H), 4.43-3.80 (m, 9H), 2.34-2.25 (m, 3H), 2.13 (s, 3H), 1.96-1.87 (m, 3H), 1.37-1.31 (m, 1H), 1.21-098 (m, 7H), 0.81-0.70 (m, 1H). Second eluting diastereomer (33 mg, I-2) as a white solid. LCMS m/z=583.4 [M+H]⁺; ¹H NMR (400 MHz, CD₃OD) δ 8.25-8.17 (m, 1H), 7.97-7.88 (m, 1H), 7.40-7.24 (m, 5H), 5.92-5.80 (m, 2H), 5.39 (s, 2H), 4.33-3.80 (m, 9H), 2.35-2.27 (m, 3H), 2.13 (s, 3H), 1.96-1.89 (m, 3H), 1.38-1.32 (m, 1H), 1.21-1.01 (m, 7H), 0.82-0.71 (m, 1H).

Table 15: The compounds listed in Table 15 were synthesized from 6-(1-benzyl-JH-pyrazole-4-carbonyl)-2-((S)-2,2-dimethylcyclopropane-1-carbonyl)-N′-hydroxy-2,6-diazaspiro[3.4]octane-8-carboximidamide according to the procedures outlined for I-2 using the appropriate commercially available reagents and/or intermediates described elsewhere.

TABLE 15

| Example | | | |
|---|---|---|---|
| Number | Compound | ¹HNMR | LCMS |
| I-21 | 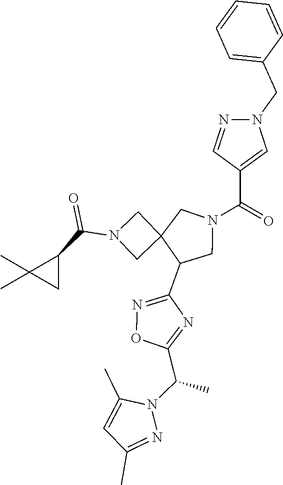 [SMILES: Cc1cc(C)n([C@@H](C)c2nc(C3CN(C(=O)c4cnn(Cc5ccccc5)c4)CC34CN(C(=O)[C@H]3CC3(C)C)C4)no2)n1] | ¹H NMR (400 MHz, CD₃OD) δ 8.28-8.17 (m, 1H), 8.00-7.86 (m, 1H), 7.50-7.25 (m, 5H), 5.97- 5.80 (m, 2H), 5.41-5.3 (m, 2H), 4.56-3.78 (m, 8H), 2.35-2.27 (m, 3H), 2.17- 2.10 (m, 3H), 1.97-1.87 (m, 3H), 1.47-1.31 (m, 1H), 1.23-0.92 (m, 8H), 0.81-0.74 (m, 1H). | m/z = 583.5 [M + H]⁺ |
| I-3 | 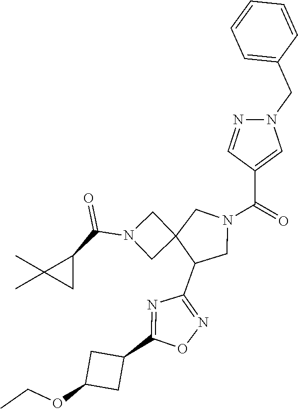 [SMILES: CCO[C@H]1C[C@@H](c2nc(C3CN(C(=O)c4cnn(Cc5ccccc5)c4)CC34CN(C(=O)[C@H]3CC3(C)C)C4)no2)C1] | ¹H NMR (400 MHz, CD₃OD) δ 8.24 (d, J = 13.5 Hz, 1H), 7.94 (d, J = 10.7 Hz, 1H), 7.37-7.26 (m, 5H), 5.38 (d, J = 2.7 Hz, 2H), 4.65-3.97 (m, 8H), 3.95-3.78 (m, 2H), 3.45 (q, J = 7.0 Hz, 2H), 3.39- .334 (m, 1H), 2.74 (d, J = 8.1 Hz, 2H), 2.28 (s, 2H), 1.48-1.26 (m, 2H), 1.23- 0.96 (m, 10H), 0.83-0.70 (m, 1H). | m/z = 559.4 [M + H]⁺ |
| I-1 | 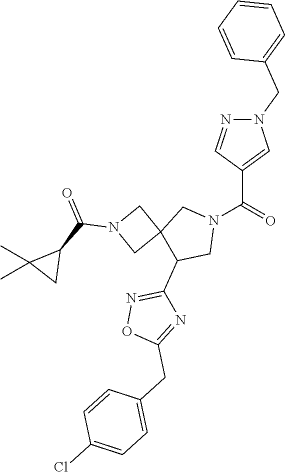 [SMILES: CC1(C)C[C@@H]1C(=O)N1CC2(CN(C(=O)c3cnn(Cc4ccccc4)c3)CC2c2noc(Cc3ccc(Cl)cc3)n2)C1] | ¹H NMR (400 MHz, CD₃OD) δ 8.24 (d, J = 16.8 Hz, 3H), 7.95 (d, J = 13.4 Hz, 3H), 7.40-7.27 (m, 27H), 5.40 (d, J = 4.8 Hz, 6H), 4.34-3.80 (m, 32H), 2.36 (s, 1H), 1.43-1.28 (m, 7H), 1.21-1.08 (m, 14H), 1.02-0.92 (m, 7H). | m/z = 585.3 [M + H]⁺ |
| I-85 | 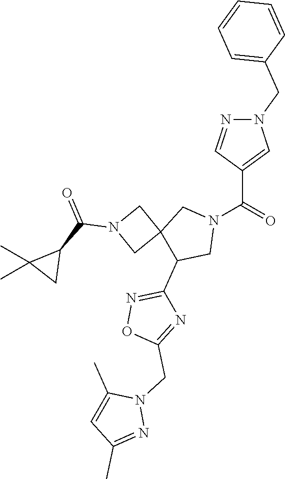 [SMILES: Cc1cc(C)n(Cc2nc(C3CN(C(=O)c4cnn(Cc5ccccc5)c4)CC34CN(C(=O)[C@H]3CC3(C)C)C4)no2)n1] | ¹H NMR (400 MHz, CD3OD) δ 8.21 (d, J = 16.9 Hz, 1H), 7.91 (d, J = 13.5 Hz, 1H), 7.38-7.25 (m, 5H), 5.90 (d, J = 9.4 Hz, 1H), 5.52 (dt, J = 4.8, 2.8 Hz, 2H), 5.38 (d, J = 4.4 Hz, 2H), 4.38-4.24 (m, 1H), 4.16 (td, J = 14.2, 13.2, 5.7 Hz, 3H), 4.05- 3.80 (m, 5H), 2.30-2.26 (m, 3H), 2.13 (s, 3H), 1.46- 1.33 (m, 1H), 1.21-0.99 (m, 7H), 0.81-0.69 (m, 1H). | m/z = 569.4 [M + H]⁺ |
| I-84 | 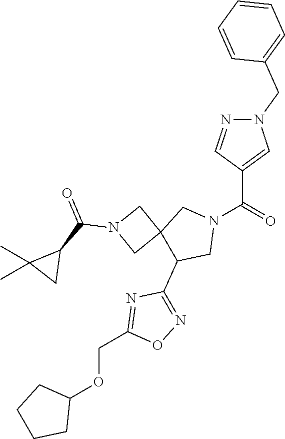 [SMILES: CC1(C)C[C@@H]1C(=O)N1CC2(CN(C(=O)c3cnn(Cc4ccccc4)c3)CC2c2noc(COC3CCCC3)n2)C1] | ¹H NMR (400 MHz, Methanol-d4) δ 8.27-8.21 (m, 1H), 7.94 (d, J = 12.3 Hz, 1H), 7.38-7.25 (m, 5H), 5.38 (d, J = 3.1 Hz, 2H), 4.72-4.70 (m, 2H), 4.44-4.16 (m, 4H), 4.13- 3.97 (m, 4H), 3.96-3.84 (m, 2H), 1.75-1.68 (m, 6H), 1.58-1.51 (m, 2H), 1.45-1.35 (m, 1H), 1.22- 1.06 (m, 6H), 1.04-0.99 (m, 2H), 0.81-0.70 (m, 1H). | m/z = 559.4 [M + H]⁺ |

### Synthesis of (8-(5-(1-(3,5-dimethyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(thiazol-5-yl)methanone (I-9)

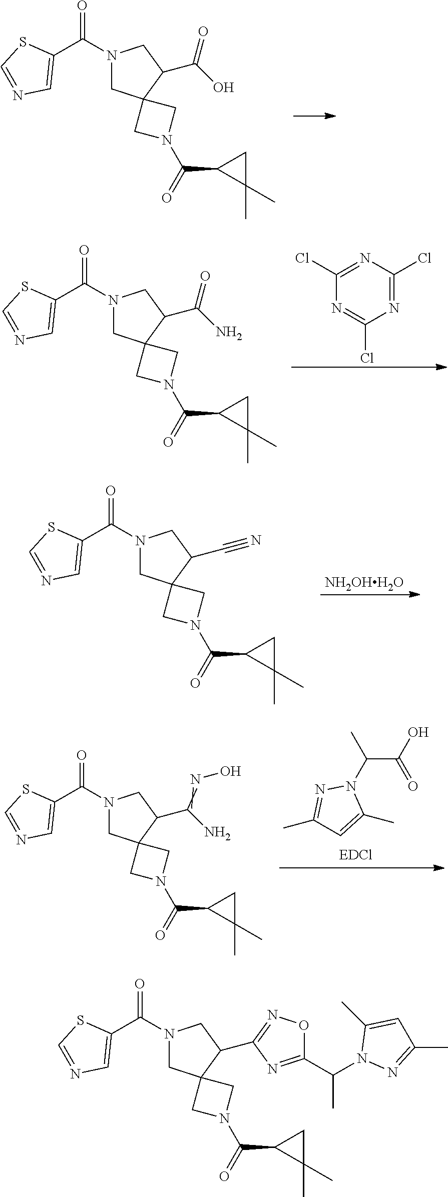

### Step 1: 2-((S)-2,2-dimethylcyclopropane-1-carbonyl)-6-(thiazole-5-carbonyl)-2,6-diazaspiro[3.4]octane-8-carboxamide

To a solution of 2-((S)-2,2-dimethylcyclopropane-1-carbonyl)-6-(thiazole-5-carbonyl)-2,6-diazaspiro[3.4]octane-8-carboxylic acid (10.0 g, 27.5 mmol) in DMF (100 mL) was added NH₄Cl (4.42 g, 82.6 mmol), EDCI (7.91 g, 41.3 mmol), HOBt (5.58 g, 41.3 mmol) and DIPEA (10.7 g, 82.6 mmol). The resulting mixture was stirred at room temperature for 48 h then the solvent was removed under reduced pressure. The residue obtained was purified by RP-column (24% MeCN in water) to afford 2-((S)-2,2-dimethylcyclopropane-1-carbonyl)-6-(thiazole-5-carbonyl)-2,6-diazaspiro[3.4]octane-8-carboxamide (9.8 g, 98%) as a light yellow solid. LCMS m/z=363.1[M+H]⁺; ¹H NMR (400 MHz, DMSO-d6) δ 9.25 (s, 1H), 8.37 (dd, J=13.2, 2.8 Hz, 1H), 7.70 (d, J=9.6 Hz, 1H), 7.20 (s, 1H), 4.40-3.60 (m, 8H), 3.24-3.02 (m, 1H), 1.41-1.27 (m, 1H), 1.15-1.01 (m, 6H), 0.89-0.82 (m, 1H), 0.71-0.62 (m, 1H).

### Step 2: 2-((S)-2,2-dimethylcyclopropane-1-carbonyl)-6-(thiazole-5-carbonyl)-2,6-diazaspiro[3.4]octane-8-carbonitrile

To a solution of 2-((S)-2,2-dimethylcyclopropane-1-carbonyl)-6-(thiazole-5-carbonyl)-2,6-diazaspiro[3.4]octane-8-carboxamide (8.8 g, 24.3 mmol) in DMF (80 mL) at 0° C. was added 2,4,6-trichloro-1,3,5-triazine (4.92 g, 26.7 mmol). The reaction was stirred at 0° C. for 2 h then diluted with water and extracted with EtOAc (200 mL×3). The combined organic layers were washed with water and brine, dried over Na₂SO₄ and concentrated. The residue was purified by column chromatography on silica gel (eluent: DCM:MeOH=20:1) to afford 2-((S)-2,2-dimethylcyclopropane-1-carbonyl)-6-(thiazole-5-carbonyl)-2,6-diazaspiro[3.4]octane-8-carbonitrile (2.5 g, 30%) as a white solid. LCMS m/z=345.1 [M+H]⁺; ¹H NMR (400 MHz, DMSO-d6) δ 9.28 (s, 1H), 8.47-8.34 (m, 1H), 4.39-3.70 (m, 9H), 1.47-1.32 (m, 1H), 1.16-1.04 (m, 6H), 0.91-0.84 (m, 1H), 0.74-0.65 (m, 1H).

### Step 3: 2-((S)-2,2-dimethylcyclopropane-1-carbonyl)-N′-hydroxy-6-(thiazole-5-carbonyl)-2,6-diazaspiro[3.4]octane-8-carboximidamide

To a solution of 2-((S)-2,2-dimethylcyclopropane-1-carbonyl)-6-(thiazole-5-carbonyl)-2,6-diazaspiro[3.4]octane-8-carbonitrile (500 mg, 1.45 mmol) in EtOH (5.0 mL) was added NH₂OH·H₂O (297 mg, 2.91 mmol). The resulting mixture was stirred at room temperature for 3 h then the solvent was removed under reduced pressure to afford 2-((S)-2,2-dimethylcyclopropane-1-carbonyl)-N′-hydroxy-6-(thiazole-5-carbonyl)-2,6-diazaspiro[3.4]octane-8-carboximidamide (550 mg, quant.) as a white solid which was used without further purification. LCMS m/z=378.3 [M+H]⁺; ¹H NMR (400 MHz, DMSO-d6) δ 9.25 (s, 1H), 9.17 (t, J=5.2 Hz, 1H), 8.41-8.30 (m, 1H), 5.63 (d, J=6.8 Hz, 2H), 4.46-3.57 (m, 9H), 1.35 (t, J=6.2 Hz, 1H), 1.15-1.01 (m, 7H), 0.85 (s, 1H), 0.73-0.57 (m, 1H).

### Step 4: (8-(5-(1-(3,5-dimethyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(thiazol-5-yl)methanone

To a solution of 2-(3,5-dimethyl-1H-pyrazol-1-yl)propanoic acid (34 mg, 0.2 mmol) in a mixture of DMF and 1,4-dioxane (2 mL and 0.5 mL) was ECDI (39 mg, 0.2 mmol) and 2-((S)-2,2-dimethylcyclopropane-1-carbonyl)-N′-hydroxy-6-(thiazole-5-carbonyl)-2,6-diazaspiro[3.4]octane-8-carboximidamide (38 mg, 0.1 mmol). The reaction was heated at 60° C. for 6 h, then the temperature increased to 100° C. and heating continued for 14 h. The solvent was removed and the residue obtained purified by prep-HPLC to afford (8-(5-(1-(3,5-dimethyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(thiazol-5-yl)methanone (8 mg, 16%) as a colorless solid. LCMS m/z=510.2 [M+H]⁺; ¹H NMR (400 MHz, CD₃OD) δ 9.17 (d, J=4.8 Hz, 1H), 8.44-8.32 (m, 1H), 5.95-5.89 (m, 1H), 5.88-5.80 (m, 1H), 4.50-4.15 (m, 4H), 4.14-3.78 (m, 5H), 2.37-2.29 (m, 3H), 2.18-2.11 (m, 3H), 1.98-1.90 (m, 3H), 1.49-1.36 (m, 1H), 1.21-0.99 (m, 7H), 0.84-0.72 (m, 1H).

Table 16: The compounds listed in Table 16 were synthesized from 2-((S)-2,2-dimethylcyclopropane-1-carbonyl)-N′-hydroxy-6-(thiazole-5-carbonyl)-2,6-diazaspiro[3.4]octane-8-carboximidamide according to the procedures outlined for I-9 using the appropriate commercially available reagents and/or intermediates described elsewhere.

TABLE 16

| Example | | | |
|---|---|---|---|
| Number | Compound | ¹HNMR | LCMS |
| I-64 | 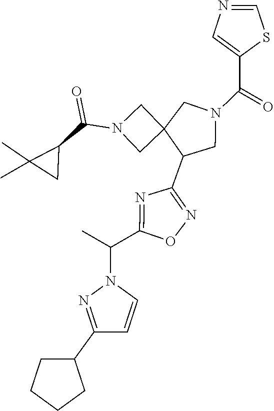 [SMILES: CC(c1nc(C2CN(C(=O)c3cncs3)CC23CN(C(=O)[C@H]2CC2(C)C)C3)no1)n1ccc(C2CCCC2)n1] | ¹H NMR (400 MHz, CD₃OD) δ 9.18-9.12 (m, 1H), 8.44-8.28 (m, 1H), 7.66-7.60 (m, 1H), 7.39 (s, 1H), 5.94-5.81 (m, 1H), 4.62-3.83 (m, 9H), 2.97-2.85 (m, 1H), 2.12-1.87 (m, 5H), 1.83- 1.58 (m, 4H), 1.57- 1.26 (m, 3H), 1.22-0.97 (m, 7H), 0.81-0.71 (m, 1H). | m/z = 550.3 [M + H]⁺ |
| I-82 | 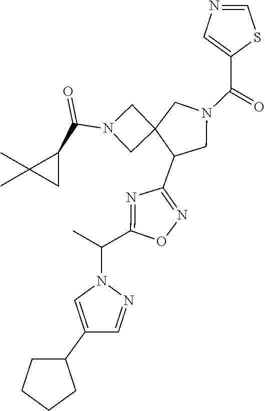 | ¹H NMR (400 MHz, Methanol-d₄) δ 9.16 (d, J = 4.1 Hz, 1H), 8.37 (d, J = 9.0 Hz, 1H), 7.79- 7.67 (m, 1H), 6.26-6.15 (m, 1H), 5.97-5.83 (m, 1H), 4.60-3.78 (m, 11H), 3.58-3.46 (m, 2H), 2.92-2.79 (m, 1H), 2.01-1.91 (m, 3H), 1.87- 1.64 (m, 4H), 1.51- 1.27 (m, 1H), 1.24-0.94 (m, 7H), 0.85-0.69 (m, 1H). | m/z = 566.3 [M + H]⁺ |
| I-100 | 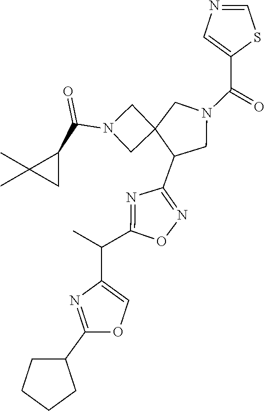 [SMILES: CC(c1coc(C2CCCC2)n1)c1nc(C2CN(C(=O)c3cncs3)CC23CN(C(=O)[C@H]2CC2(C)C)C3)no1] | ¹H NMR (400 MHz, DMSO-d₆) δ 9.16 (d, J = 3.8 Hz, 1H), 8.38 (d, J = 9.0 Hz, 1H), 7.83-7.66 (m, 1H), 4.62-3.80 (m, 11H), 3.23-3.12 (m, 1H), 2.14-1.96 (m, 2H), 1.89-1.76 (m, 3H), 1.73- 1.56 (m, 5H), 1.45- 1.31 (m, 2H), 1.22-1.15 (m, 2H), 1.14-0.98 (m, 5H), 0.81-0.68 (m, 1H). | m/z = 551.3 [M + H]⁺ |
| I-95 | 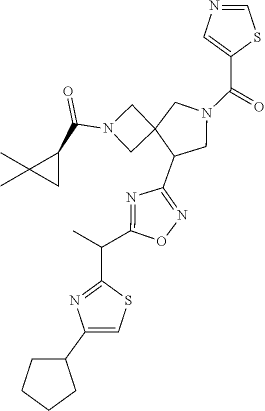 [SMILES: CC(c1nc(C2CN(C(=O)c3cncs3)CC23CN(C(=O)[C@H]2CC2(C)C)C3)no1)c1nc(C2CCCC2)cs1] | ¹H NMR (400 MHz, Methanol-d4) δ 9.16 (d, J = 3.6 Hz, 1H), 8.38 (d, J = 7.6 Hz, 1H), 7.32- 7.05 (m, 1H), 4.71-4.62 (m, 1H), 4.57 (s, 1H), 4.42-3.83 (m, 9H), 3.46-3.35 (m, 1H), 2.21- 2.11 (m, 2H), 1.84-1.68 (m, 9H), 1.44-1.33 (m, 1H), 1.20-0.99 (m, 7H), 0.75 (s, 1H). | m/z = 567.3 [M + H]⁺ |
| I-81 | 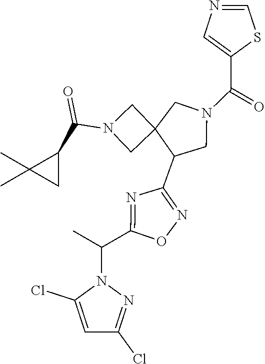 [SMILES: CC(c1nc(C2CN(C(=O)c3cncs3)CC23CN(C(=O)[C@H]2CC2(C)C)C3)no1)n1nc(Cl)cc1Cl] | ¹H NMR (400 MHz, Methanol-d4) δ 9.17 (d, J = 4.6 Hz, 1H), 8.43- 8.31 (m, 1H), 6.49-6.27 (m, 1H), 6.18-6.02 (m, 1H), 4.62-3.79 (m, 9H), 2.01-1.89 (m, 3H), 1.50- 1.27 (m, 1H), 1.25- 0.98 (m, 7H), 0.84-0.69 (m, 1H). | m/z = 550.2 [M + H]⁺ |
| I-62 | 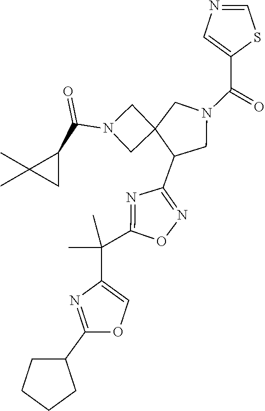 [SMILES: CC(C)(c1coc(C2CCCC2)n1)c1nc(C2CN(C(=O)c3cncs3)CC23CN(C(=O)[C@H]2CC2(C)C)C3)no1] | ¹H NMR (400 MHz, Methanol-d₄) δ 9.16 (d, J = 3.6 Hz, 1H), 8.49- 8.31 (m, 1H), 7.80-7.61 (m, 1H), 4.62-3.81 (m, 9H), 3.25-3.12 (m, 1H), 2.09-1.95 (m, 2H), 1.94- 1.61 (m, 12H), 1.49- 1.34 (m, 1H), 1.23-0.93 (m, 7H), 0.82-0.68 (m, 1H). | m/z = 565.2 [M + H]⁺ |
| I-63 | 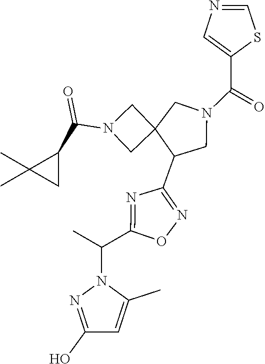 [SMILES: Cc1cc(O)nn1C(C)c1nc(C2CN(C(=O)c3cncs3)CC23CN(C(=O)[C@H]2CC2(C)C)C3)no1] | ¹H NMR (400 MHz, CD3OD) δ 9.16 (d, J = 3.5 Hz, 1H), 8.42-8.35 (m, 1H), 5.72 (p, J = 7.1 Hz, 1H), 5.41 (d, J = 4.3 Hz, 1H), 4.41-3.85 (m, 9H), 3.31-3.18 (m, 2H), 1.88 (tt, J = 6.7, 2.5 Hz, 3H), 1.50-1.33 (m, 1H), 1.11 (m, J = 25.2, 22.6, 13.8, 6.5 Hz, 8H), 0.82- 0.72 (m, 1H). | m/z = 512.3 [M + H]⁺ |
| I-75 | 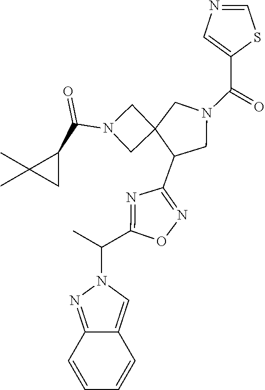 [SMILES: CC(c1nc(C2CN(C(=O)c3cncs3)CC23CN(C(=O)[C@H]2CC2(C)C)C3)no1)n1cc2ccccc2n1] | ¹H NMR (400 MHz, Methanol-d₄) δ 9.18- 9.10 (m, 1H), 8.47-8.43 (m, 1H), 8.36-8.33 (m, 1H), 7.71 (d, J = 8.4 Hz, 1H), 7.60-7.53 (m, 1H), 7.32-7.28 (m, 1H), 7.10 (t, J = 7.6 Hz, 1H), 6.37- 6.24 (m, 1H), 4.57- 3.83 (m, 9H), 2.15-2.11 (m, 3H), 1.44-1.29 (m, 1H), 1.19-0.96 (m, 7H), 0.81-0.62 (m, 1H). | m/z = 532.3 [M + H]⁺ |
| I-74 | 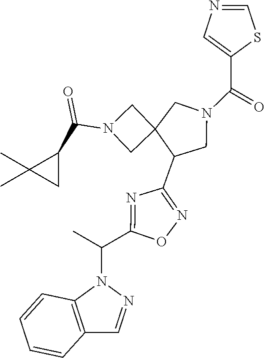 [SMILES: CC(c1nc(C2CN(C(=O)c3cncs3)CC23CN(C(=O)[C@H]2CC2(C)C)C3)no1)n1ncc2ccccc21] | ¹H NMR (400 MHz, Methanol-d₄) δ 9.16- 9.15 (m, 1H), 8.32 (d, J = 2.8 Hz, 1H), 8.06 (d, J = 3.6 Hz, 1H), 7.77 (d, J = 8.0 Hz, 1H), 7.64-7.58 (m, 1H), 7.44 (t, J = 7.8 Hz, 1H), 7.20 (t, J = 7.6 Hz, 1H), 6.38 (d, J = 3.6 Hz, 1H), 4.57-3.78 (m, 9H), 2.11-2.06 (m, 3H), 1.41-1.29 (m, 1H), 1.20- 0.96 (m, 7H), 0.78-0.74 (m, 1H). | m/z = 532.3 [M + H]⁺ |
| I-36 | 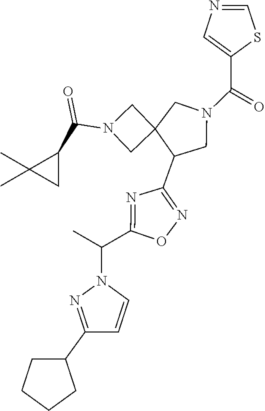 [SMILES: CC(c1nc(C2CN(C(=O)c3cncs3)CC23CN(C(=O)[C@H]2CC2(C)C)C3)no1)n1ccc(C2CCCC2)n1] | ¹H NMR (400 MHz, CD₃OD) δ 9.16 (d, J = 4.0 Hz, 1H), 8.41-8.32 (m, 1H), 7.72-7.66 (m, 1H), 6.17 (d, J = 2.5 Hz, 1H), 5.87 (d, J = 7.0 Hz, 1H), 4.60-3.82 (m, 9H), 3.08-2.98 (m, 1H), 2.06- 1.91 (m, 5H), 1.83-1.54 (m, 6H), 1.46-1.32 (m, 1H), 1.21-0.99 (m, 7H), 0.81-0.71 (m, 1H). | m/z = 550.4 [M + H]⁺ |

### Synthesis of (8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(thiazol-5-yl)methanone (I-36)

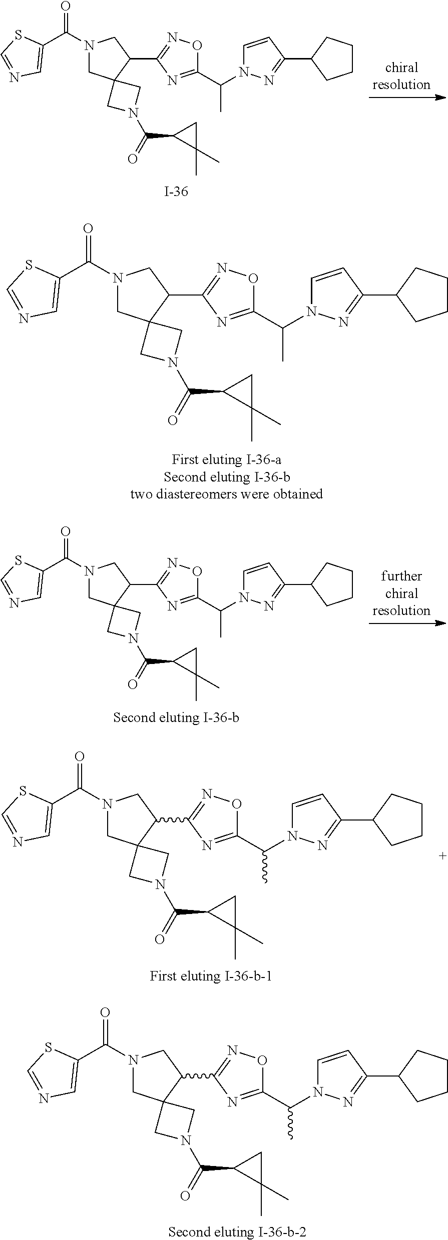

(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(thiazol-5-yl)methanone I-36 was separated by prep-HPLC to afford the two isomers. First eluting (85 mg, I-36-a), LCMS m/z=550.3 [M+H]⁺; ¹H NMR (400 MHz, Methanol-d4) δ 9.16 (d, J=4.2 Hz, 1H), 8.37 (d, J=9.0 Hz, 1H), 7.70 (d, J=8.5 Hz, 1H), 6.17 (d, J=2.5 Hz, 1H), 5.91-5.82 (m, 1H), 4.45-3.69 (m, 9H), 3.14-2.90 (m, 1H), 2.04-1.55 (m, 12H), 1.41 (d, J=5.6 Hz, 1H), 1.24-0.86 (m, 8H), 0.82-0.71 (m, 1H). Second eluting (62 mg, I-36-b), LCMS m/z=550.2 [M+H]⁺; ¹H NMR (400 MHz, Methanol-d4) δ 9.16 (d, J=4.1 Hz, 1H), 8.41-8.34 (m, 1H), 7.69 (d, J=9.1 Hz, 1H), 6.17 (d, J=2.4 Hz, 1H), 5.94-5.82 (m, 1H), 4.57 (s, 1H), 4.36-3.82 (m, 9H), 3.04 (q, J=8.3 Hz, 1H), 1.96 (t, J=11.4 Hz, 6H), 1.83-1.57 (m, 6H), 1.45-1.35 (m, 1H), 1.22-0.96 (m, 8H), 0.82-0.71 (m, 1H).

Chiral HPLC purification of the second eluting isomer above provided the two enantiomers: First eluting (18 mg, I-36-b-1), LCMS m/z=550.2 [M+H]⁺; ¹H NMR (400 MHz, Methanol-d4) δ 9.16 (d, J=4.4 Hz, 1H), 8.37 (d, J=8.7 Hz, 1H), 7.69 (d, J=9.9 Hz, 1H), 6.17 (s, 1H), 5.95-5.80 (m, 1H), 4.62-3.82 (m, 10H), 3.12-2.90 (m, 1H), 2.10-1.52 (m, 13H), 1.47-1.32 (m, 2H), 1.22-0.99 (m, 8H), 0.82-0.71 (m, 1H). Second eluting (12 mg, I-36-b-2), LCMS m/z=550.3 [M+H]⁺; ¹H NMR (400 MHz, Methanol-d4) δ 9.16 (d, J=3.6 Hz, 1H), 8.48-8.27 (m, 1H), 7.80-7.61 (m, 1H), 6.17 (s, 1H), 5.99-5.72 (m, 1H), 4.57-3.85 (m, 10H), 3.07-2.97 (m, 1H), 2.04-1.56 (m, 12H), 1.46-1.36 (m, 1H), 1.20-1.02 (m, 7H), 0.82-0.71 (m, 1H).

Conditions for separating the two isomers by chiral HPLC and Retention time for each isomer (and LCMS).

Chiral prep-HPLC:

| | |
|---|---|
| Column | CHIRALPAK AD-H(ADH0CE-XG136) |
| Column size | 0.46 cm I.D. × 25 cm L |
| Mobile phase | Hexane/IPA/DEA = 70/30/0.1(V/V/V) |
| Flow rate | 1.0 ml/min |
| Wave length | UV 254 m |

Retention time for I-36-b-1: 10.9 min
Retention time for I-36-b-2: 12.5 min

### Synthesis of (8-(5-(1-(1H-pyrazol-3-yl)ethyl)-1,2,4-oxadiazol-3-yl)-2-((S)-2,2-dimethylcyclopropane-1-carbonyl)-2,6-diazaspiro[3.4]octan-6-yl)(thiazol-5-yl)methanone (I-77)

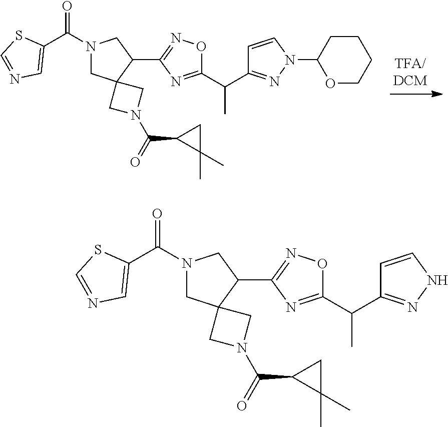 [SMILES: CC(c1ccn(C2CCCCO2)n1)c1nc(C2CN(C(=O)c3cncs3)CC23CN(C(=O)[C@H]2CC2(C)C)C3)no1 ; CC(c1cc[nH]n1)c1nc(C2CN(C(=O)c3cncs3)CC23CN(C(=O)[C@H]2CC2(C)C)C3)no1]

(2-((S)-2,2-dimethylcyclopropane-1-carbonyl)-8-(5-(1-(1-(tetrahydro-2H-pyran-2-yl)-1H-pyrazol-3-yl)ethyl)-1,2,4-oxadiazol-3-yl)-2,6-diazaspiro[3.4]octan-6-yl)(thiazol-5-yl)methanone: (2-((S)-2,2-dimethylcyclopropane-1-carbonyl)-8-(5-(1-(1-(tetrahydro-2H-pyran-2-yl)-1H-pyrazol-3-yl)ethyl)-1,2,4-oxadiazol-3-yl)-2,6-diazaspiro[3.4]octan-6-yl)(thiazol-5-yl)methanone was synthesized from 2-((S)-2,2-dimethylcyclopropane-1-carbonyl)-N′-hydroxy-6-(thiazole-5-carbonyl)-2,6-diazaspiro[3.4]octane-8-carboximidamide according to the procedures outlined for I-9 using the appropriate commercially available reagents and/or intermediates described elsewhere. LCMS m/z=567.1 [M+H]⁺.

### Step 1: (8-(5-(1-(1H-pyrazol-3-yl)ethyl)-1,2,4-oxadiazol-3-yl)-2-((S)-2,2-dimethylcyclopropane-1-carbonyl)-2,6-diazaspiro[3.4]octan-6-yl)(thiazol-5-yl)methanone

To a solution of (2-((S)-2,2-dimethylcyclopropane-1-carbonyl)-8-(5-(1-(1-(tetrahydro-2H-pyran-2-yl)-1H-pyrazol-3-yl)ethyl)-1,2,4-oxadiazol-3-yl)-2,6-diazaspiro[3.4]octan-6-yl)(thiazol-5-yl)methanone (5 mg, 8.8 umol) in DCM (0.5 mL) was added TFA (0.2 mL) and the mixture stirred at room temperature for 4 h. The solvent was removed under reduced pressure and the residue obtained purified by prep-HPLC to afford (8-(5-(1-(1H-pyrazol-3-yl)ethyl)-1,2,4-oxadiazol-3-yl)-2-((S)-2,2-dimethylcyclopropane-1-carbonyl)-2,6-diazaspiro[3.4]octan-6-yl)(thiazol-5-yl)methanone (1 mg, 23%) as a white solid. LCMS m/z=482.3 [M+H]⁺; ¹H NMR (400 MHz, CD₃OD) δ 9.15 (d, J=4.3 Hz, 1H), 8.40-8.28 (m, 1H), 8.03 (m, J=45.9, 31.7, 10.6, 6.9 Hz, 2H), 4.50-3.81 (m, 9H), 3.56-3.38 (m, 1H), 2.77 (m, J=11.2, 9.5, 7.0 Hz, 6H), 1.41 (m, J=34.3, 10.8, 6.3 Hz, 1H), 1.21-1.07 (m, 6H), 1.07-0.99 (m, 1H), 0.93 (m, J=23.1, 5.0 Hz, 1H), 0.82-0.69 (m, 1H).

Table 17: The compounds listed in Table 17 were synthesized from 2-((S)-2,2-dimethylcyclopropane-1-carbonyl)-N′-hydroxy-6-(thiazole-5-carbonyl)-2,6-diazaspiro[3.4]octane-8-carboximidamide according to the procedures outlined for I-77 using the appropriate commercially available reagents and/or intermediates described elsewhere.

TABLE 17

| Example | | | |
|---|---|---|---|
| Number | Compound | ¹HNMR | LCMS |
| I-79 | 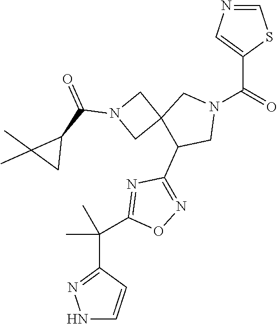 [SMILES: CC(C)(c1cc[nH]n1)c1nc(C2CN(C(=O)c3cncs3)CC23CN(C(=O)[C@H]2CC2(C)C)C3)no1] | ¹H NMR (400 MHz, CD₃OD) δ 9.16 (d, J = 4.1 Hz, 1H), 8.39 (d, J = 8.3 Hz, 1H), 7.58-7.54 (m, 1H), 6.25 (dq, J = 8.1, 3.3, 2.6 Hz, 1H), 4.57 (s, 2H), 4.43-3.82 (m, 9H), 1.81 (d, J = 4.0 Hz, 6H), 1.42 (dd, J = 8.1, 5.7 Hz, 1H), 1.21- 0.98 (m, 7H), 0.81-0.70 (m, 1H). | m/z = 496.3 [M + H]⁺ |

### Synthesis of (8-(5-(4-chloro-3-(trifluoromethyl)benzyl)oxazol-2-yl)-2-((S)-2,2-dimethylcyclopropane-1-carbonyl)-2,6-diazaspiro[3.4]octan-6-yl)(thiazol-5-yl)methanone (I-48)

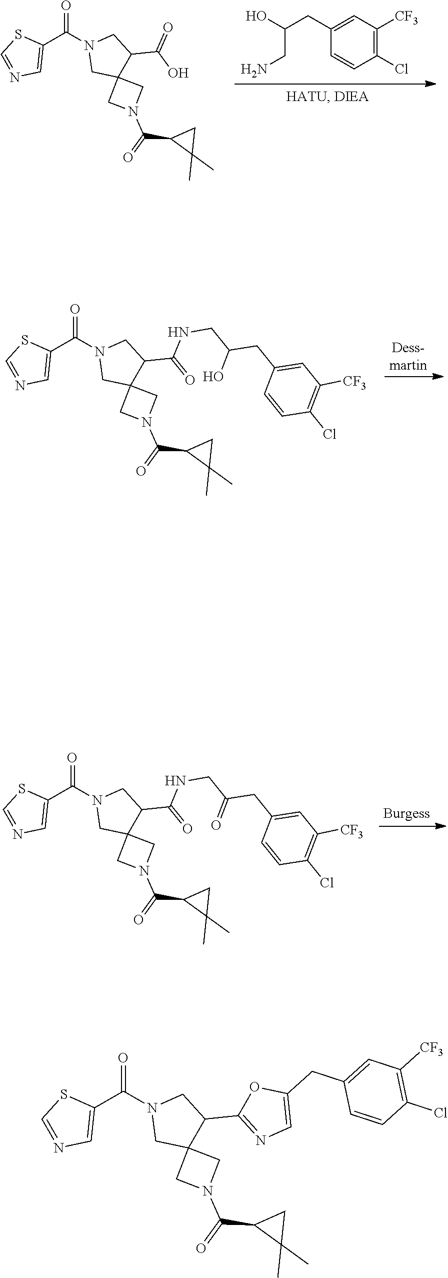 [SMILES: CC1(C)C[C@@H]1C(=O)N1CC2(CN(C(=O)c3cncs3)CC2C(=O)O)C1 ; NCC(O)Cc1ccc(Cl)c(C(F)(F)F)c1 ; CC1(C)C[C@@H]1C(=O)N1CC2(CN(C(=O)c3cncs3)CC2C(=O)NCC(O)Cc2ccc(Cl)c(C(F)(F)F)c2)C1 ; CC1(C)C[C@@H]1C(=O)N1CC2(CN(C(=O)c3cncs3)CC2C(=O)NCC(=O)Cc2ccc(Cl)c(C(F)(F)F)c2)C1 ; CC1(C)C[C@@H]1C(=O)N1CC2(CN(C(=O)c3cncs3)CC2c2ncc(Cc3ccc(Cl)c(C(F)(F)F)c3)o2)C1]

### Step 1: N-(3-(4-chloro-3-(trifluoromethyl)phenyl)-2-hydroxypropyl)-2-((S)-2,2-dimethylcyclopropane-1-carbonyl)-6-(thiazole-5-carbonyl)-2,6-diazaspiro[3.4]octane-8-carboxamide

To a solution of 1-amino-3-(4-chloro-3-(trifluoromethyl)phenyl)propan-2-ol (300 mg, 0.86 mmol) in DMF (16 mL) was added HATU (470 mg, 1.24 mmol) and DIPEA (320 mg, 2.48 mmol). The reaction was stirred at room temperature for 30 min then 2-((S)-2,2-dimethylcyclopropane-1-carbonyl)-6-(thiazole-5-carbonyl)-2,6-diazaspiro[3.4]octane-8-carboxylic acid (250 mg, 0.99 mmol) was added and the reaction stirred at room temperature overnight. The mixture was diluted with water (30 mL) and extracted with EtOAc (50 mL×3). The combined organic layers were washed with brine, dried over Na₂SO₄, filtered and concentrated to afford the N-(3-(4-chloro-3-(trifluoromethyl)phenyl)-2-hydroxypropyl)-2-((S)-2,2-dimethylcyclopropane-1-carbonyl)-6-(thiazole-5-carbonyl)-2,6-diazaspiro[3.4]octane-8-carboxamide (350 mg, 70.8%) as a yellow solid which was used without further purification. LCMS m/z=599.3 [M+H]⁺.

### Step 2: N-(3-(4-chloro-3-(trifluoromethyl)phenyl)-2-oxopropyl)-2-((S)-2,2-dimethylcyclopropane-1-carbonyl)-6-(thiazole-5-carbonyl)-2,6-diazaspiro[3.4]octane-8-carboxamide

To a solution of N-(3-(4-chloro-3-(trifluoromethyl)phenyl)-2-hydroxypropyl)-2-((S)-2,2-dimethylcyclopropane-1-carbonyl)-6-(thiazole-5-carbonyl)-2,6-diazaspiro[3.4]octane-8-carboxamide (290 mg, 0.62 mmol) in DCM (10 mL) at 0° C. was added Dess-Martin reagent (654 mg, 1.5 mmol). The reaction was stirred at room temperature overnight then was diluted with water (35 mL) and extracted with DCM (50 mL×3). The combined organic layers were washed with brine, dried over Na₂SO₄, filtered and concentrated. The residue obtained was purified by prep-TLC (eluent: DCM:MeOH=20:1) to afford N-(3-(4-chloro-3-(trifluoromethyl)phenyl)-2-oxopropyl)-2-((S)-2,2-dimethylcyclopropane-1-carbonyl)-6-(thiazole-5-carbonyl)-2,6-diazaspiro[3.4]octane-8-carboxamide (87 mg, 65%) as a yellow oil. LCMS m/z=597.2 [M+H]⁺; ¹H NMR (400 MHz, DMSO-d₆) δ 9.25 (1H, s), 8.59 (1H, dd, J=12.0, 6.0 Hz), 8.32-8.43 (1H, m), 7.65-7.71 (3H, m), 7.49 (1H, d, J=8.0 Hz), 3.66-4.28 (12H, m), 3.17 (1H, s), 1.91 (1H, s), 1.20-1.42 (2H, m), 0.57-1.15 (12H, m).

### Step 3: (8-(5-(4-chloro-3-(trifluoromethyl)benzyl)oxazol-2-yl)-2-((S)-2,2-dimethylcyclopropane-1-carbonyl)-2,6-diazaspiro[3.4]octan-6-yl)(thiazol-5-yl)methanone

To a solution of N-(3-(4-chloro-3-(trifluoromethyl)phenyl)-2-hydroxypropyl)-2-((S)-2,2-dimethylcyclopropane-1-carbonyl)-6-(thiazole-5-carbonyl)-2,6-diazaspiro[3.4]octane-8-carboxamide (80 mg, 0.13 mmol) in DCE (2 mL) was added Burgess reagent (255 mg, 1.1 mmol). The reaction was heated at 120° C. in the microwave for 1 h then filtered through Celite and the filtrate concentrated. The residue obtained was purified by prep-HPLC to afford (8-(5-(4-chloro-3-(trifluoromethyl)benzyl)oxazol-2-yl)-2-((S)-2,2-dimethylcyclopropane-1-carbonyl)-2,6-diazaspiro[3.4]octan-6-yl)(thiazol-5-yl)methanone (17 mg, 22%) as yellow solid. LCMS m/z=579.2 [M+H]⁺; ¹H NMR (CD₃OD, 400 MHz) δ 9.16 (1H, s), 8.37 (1H, s), 7.69 (1H, s), 7.53 (OH, s), 7.47 (1H, s), 6.88-6.97 (10H, m), 3.80-4.48 (9H, m), 1.33 (OH, d, J=6.8 Hz), 1.06-1.25 (4H, m), 0.85-1.06 (2H, m), 0.74 (1H, d, J=11.6 Hz).

### Synthesis of (1-benzyl-1H-pyrazol-4-yl)(8-(5-(4-chlorobenzyl)oxazol-2-yl)-2-((S)-2,2-dimethylcyclopropane-1-carbonyl)-2,6-diazaspiro[3.4]octan-6-yl)methanone (I-8)

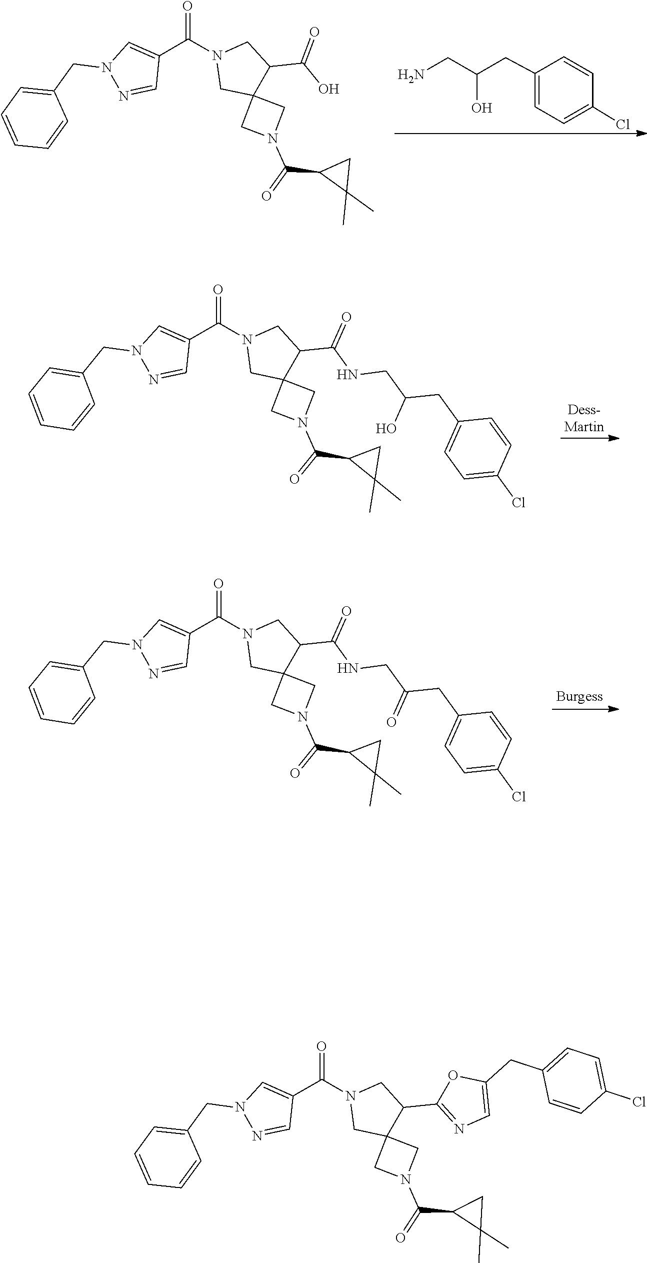 [SMILES: CC1(C)C[C@@H]1C(=O)N1CC2(CN(C(=O)c3cnn(Cc4ccccc4)c3)CC2C(=O)O)C1 ; NCC(O)Cc1ccc(Cl)cc1 ; CC1(C)C[C@@H]1C(=O)N1CC2(CN(C(=O)c3cnn(Cc4ccccc4)c3)CC2C(=O)NCC(O)Cc2ccc(Cl)cc2)C1 ; CC1(C)C[C@@H]1C(=O)N1CC2(CN(C(=O)c3cnn(Cc4ccccc4)c3)CC2C(=O)NCC(=O)Cc2ccc(Cl)cc2)C1 ; CC1(C)C[C@@H]1C(=O)N1CC2(CN(C(=O)c3cnn(Cc4ccccc4)c3)CC2c2ncc(Cc3ccc(Cl)cc3)o2)C1]

### Step 1: 6-(1-benzyl-1H-pyrazole-4-carbonyl)-N-(3-(4-chlorophenyl)-2-hydroxypropyl)-2-((S)-2,2-dimethylcyclopropane-1-carbonyl)-2,6-diazaspiro[3.4]octane-8-carboxamide (A-0700-04)

To a solution of 1-amino-3-(4-chlorophenyl)propan-2-ol (470 mg, 1.08 mmol) in DMA (20 mL) was added 6-(1-benzyl-1H-pyrazole-4-carbonyl)-2-((S)-2,2-dimethylcyclopropane-1-carbonyl)-2,6-diazaspiro[3.4]octane-8-carboxylic acid (300 mg, 1.62 mmol), EDCI (310 mg, 1.62 mmol), HOBt (218 mg, 1.62 mmol) and DIPEA (417 mg, 3.23 mmol). The reaction was stirred at room temperature overnight then diluted with water (35 mL) and extracted with EtOAc (50 mL×3). The combined organic layers were washed with brine, dried over Na₂SO₄, filtered and concentrated to afford 6-(1-benzyl-1H-pyrazole-4-carbonyl)-N-(3-(4-chlorophenyl)-2-hydroxypropyl)-2-((S)-2,2-dimethylcyclopropane-1-carbonyl)-2,6-diazaspiro[3.4]octane-8-carboxamide (554 mg, 85%) as a yellow oil. LCMS m/z=604.3 [M+H]⁺; ¹H NMR (400 MHz, Methanol-d4) δ 8.26-8.14 (m, 1H), 7.92 (d, J=11.9 Hz, 1H), 7.41-7.15 (m, 9H), 5.37 (d, J=4.7 Hz, 2H), 4.53-3.72 (m, 9H), 3.49-3.33 (m, 1H), 3.29-3.10 (m, 2H), 2.82-2.60 (m, 2H), 1.47-0.67 (m, 12H).

### Step 2: 6-(1-benzyl-1H-pyrazole-4-carbonyl)-N-(3-(4-chlorophenyl)-2-oxopropyl)-2-((S)-2,2-dimethylcyclopropane-1-carbonyl)-2,6-diazaspiro[3.4]octane-8-carboxamide

To a solution of 6-(1-benzyl-1H-pyrazole-4-carbonyl)-N-(3-(4-chlorophenyl)-2-hydroxypropyl)-2—((S)-2,2-dimethylcyclopropane-1-carbonyl)-2,6-diazaspiro[3.4]octane-8-carboxamide (550 mg, 0.91 mmol) in DCM (15 mL) was added Dess-Martin reagent (772 mg, 1.82 mmol) and the mixture stirred at room temperature for 5 h. The reaction was diluted with DCM (100 mL), the organic layer was washed with saturated aqueous NaHCO₃ and saturated aqueous Na₂S₂O₃, then dried over Na₂SO₄, filtered and concentrated to give crude 6-(1-benzyl-1H-pyrazole-4-carbonyl)-N-(3-(4-chlorophenyl)-2-oxopropyl)-2-((S)-2,2-dimethylcyclopropane-1-carbonyl)-2,6-diazaspiro[3.4]octane-8-carboxamide (450 mg, 82%). LCMS m/z=602.3 [M+H]⁺.

### Step 3: (1-benzyl-1H-pyrazol-4-yl)(8-(5-(4-chlorobenzyl)oxazol-2-yl)-2-((S)-2,2-dimethylcyclopropane-1-carbonyl)-2,6-diazaspiro[3.4]octan-6-yl)methanone

To a solution of 6-(1-benzyl-1H-pyrazole-4-carbonyl)-N-(3-(4-chlorophenyl)-2-oxopropyl)-2-((S)-2,2-dimethylcyclopropane-1-carbonyl)-2,6-diazaspiro[3.4]octane-8-carboxamide (300 mg, 0.50 mmol) in DCE (8 mL) was added Burgess reagent (356 mg, 0.996 mmol). The mixture was heated at 70° C. overnight then the solvent was removed and the residue obtained purified by prep-HPLC to give (1-benzyl-1H-pyrazol-4-yl)(8-(5-(4-chlorobenzyl)oxazol-2-yl)-2-((S)-2,2-dimethylcyclopropane-1-carbonyl)-2,6-diazaspiro[3.4]octan-6-yl)methanone (45 mg, 14%). LCMS m/z=584.2 [M+H]⁺; ¹H NMR (400 MHz, Methanol-d4) δ 8.22 (d, J=3.9 Hz, 1H), 7.93 (d, J=3.5 Hz, 1H), 7.39-7.14 (m, 9H), 6.86 (d, J=18.9 Hz, 1H), 5.38 (s, 2H), 4.33 (m, 1H), 4.21-3.90 (m, 8H), 3.89-3.79 (m, 2H), 1.43-1.25 (m, 2H), 1.20-1.11 (m, 3H), 1.11-0.98 (m, 4H), 0.98-0.87 (m, 1H), 0.81-0.67 (m, 1H).

Table 18: The compounds listed in Table 18 were synthesized from 6-(1-benzyl-1H-pyrazole-4-carbonyl)-2-((S)-2,2-dimethylcyclopropane-1-carbonyl)-2,6-diazaspiro[3.4]octane-8-carboxylic acid according to the procedures outlined for I-8 using the appropriate commercially available reagents and/or intermediates described elsewhere.

TABLE 18

| Example | | | |
|---|---|---|---|
| Number | Compound | ¹HNMR | LCMS |
| I-24 | 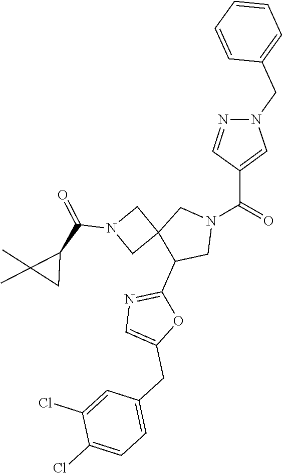 [SMILES: CC1(C)C[C@@H]1C(=O)N1CC2(CN(C(=O)c3cnn(Cc4ccccc4)c3)CC2c2ncc(Cc3ccc(Cl)c(Cl)c3)o2)C1] | ¹H NMR (400 MHz, Methanol-d4) δ 8.22 (d, J = 4.8 Hz, 1H), 7.93 (s, 1H), 7.43 (s, 2H), 7.38- 7.11 (m, 6H), 6.89 (d, J = 19.1 Hz, 1H), 5.38 (s, 2H), 4.22 (dd, J = 71.2, 7.1 Hz, 4H), 4.03 (d, J = 4.9 Hz, 5H), 3.83 (s, 2H), 1.33 (s, 1H), 1.19-0.89 (m, 8H), 0.74 (dd, J = 8.2, 4.2 Hz, 1H). | m/z = 618.4 [M + H]⁺ |

### Synthesis of (1-benzyl-1H-pyrazol-4-yl)(8-(5-(3,4-dichlorobenzyl)oxazol-2-yl)-2-((S)-2,2-dimethylcyclopropane-1-carbonyl)-2,6-diazaspiro[3.4]octan-6-yl)methanone_(I-35)

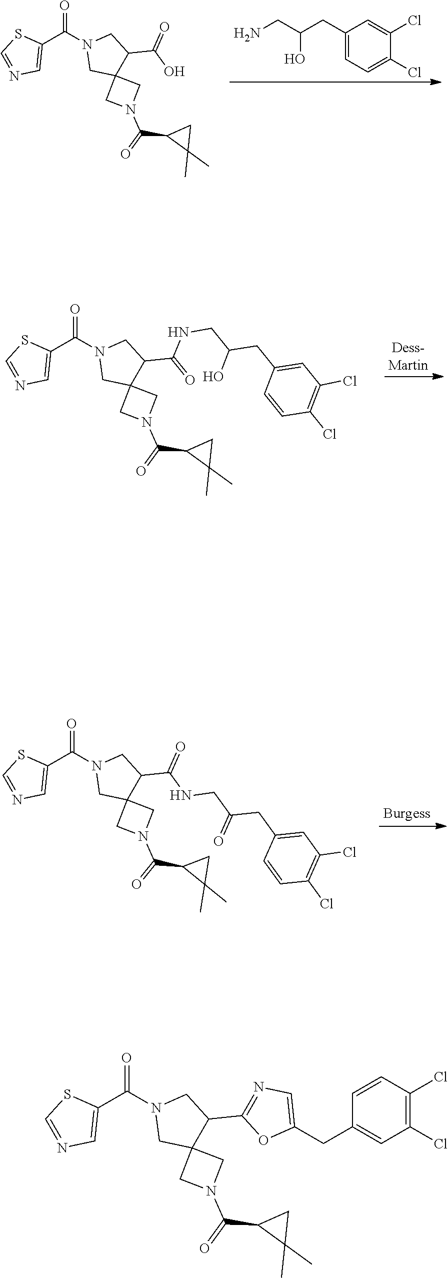 [SMILES: CC1(C)C[C@@H]1C(=O)N1CC2(CN(C(=O)c3cncs3)CC2C(=O)O)C1 ; NCC(O)Cc1ccc(Cl)c(Cl)c1 ; CC1(C)C[C@@H]1C(=O)N1CC2(CN(C(=O)c3cncs3)CC2C(=O)NCC(O)Cc2ccc(Cl)c(Cl)c2)C1 ; CC1(C)C[C@@H]1C(=O)N1CC2(CN(C(=O)c3cncs3)CC2C(=O)NCC(=O)Cc2ccc(Cl)c(Cl)c2)C1 ; CC1(C)C[C@@H]1C(=O)N1CC2(CN(C(=O)c3cncs3)CC2c2ncc(Cc3ccc(Cl)c(Cl)c3)o2)C1]

(1-benzyl-1H-pyrazol-4-yl)(8-(5-(3,4-dichlorobenzyl)oxazol-2-yl)-2-((S)-2,2-dimethylcyclopropane-1-carbonyl)-2,6-diazaspiro[3.4]octan-6-yl)methanone was synthesized from 2-((S)-2,2-dimethylcyclopropane-1-carbonyl)-6-(thiazole-5-carbonyl)-2,6-diazaspiro[3.4]octane-8-carboxylic acid according to the procedures outlined for I-8 using the appropriate commercially available reagents and/or intermediates described elsewhere. LCMS m/z=545.2 [M+H]⁺; ¹H NMR (400 MHz, Methanol-d4) δ 9.16 (s, 1H), 8.37 (s, 1H), 7.44 (dd, J=8.1, 3.1 Hz, 2H), 7.17 (s, 1H), 6.98-6.82 (m, 1H), 4.44-3.85 (m, 12H), 1.44-1.29 (m, 1H), 1.19-0.74 (m, 9H).

### Synthesis of 3-(8-(5-((3,4-dichlorophenyl)difluoromethyl)-1,3,4-oxadiazol-2-yl)-6-(thiazole-5-carbonyl)-2,6-diazaspiro[3.4]octan-2-yl)-2,2-dimethyl-3-oxopropanenitrile (I-58)

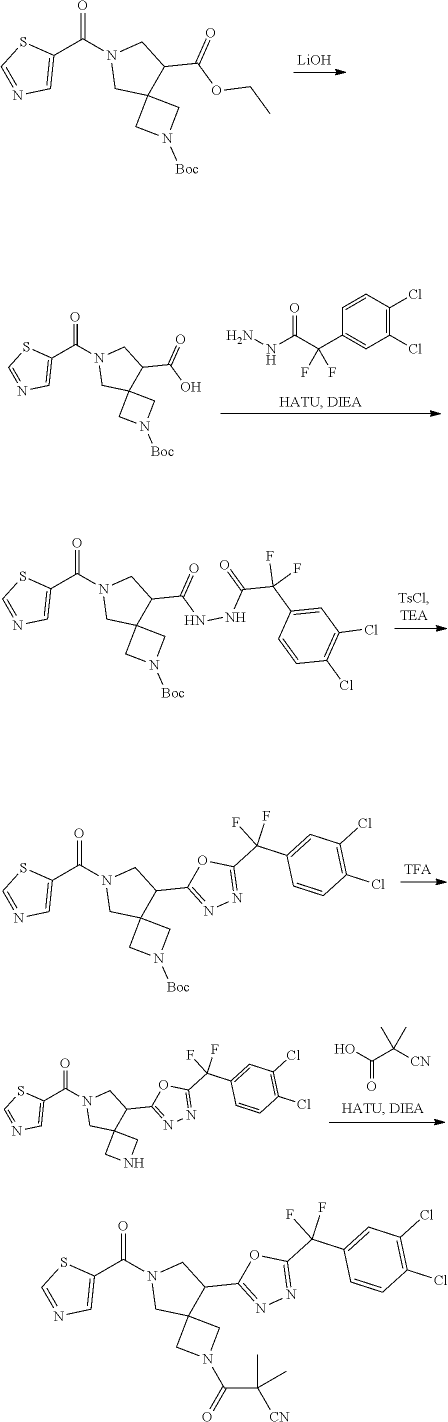

### Step 1: 2-(tert-butoxycarbonyl)-6-(thiazole-5-carbonyl)-2,6-diazaspiro[3.4]octane-8-carboxylic acid

To a solution of 2-(tert-butyl) 8-ethyl 6-(thiazole-5-carbonyl)-2,6-diazaspiro[3.4]octane-2,8-dicarboxylate (1.0 g, 2.53 mmol) in a mixture of THF and water (10 mL/2 mL) was added LiOH. H₂O (213 mg, 5.06 mmol). The mixture was stirred at room temperature for 1.5 h then diluted with water (25 mL), extracted with EtOAc (50 mL). The aqueous layer was collected and acidified to pH 1˜2 with 1M HCl then was extracted with EtOAc (100 mL×2). The combined organic layers were washed with brine, dried over Na₂SO₄, filtered and concentrated to afford 2-(tert-butoxycarbonyl)-6-(thiazole-5-carbonyl)-2,6-diazaspiro[3.4]octane-8-carboxylic acid (600 mg, 65%) as a white solid. LCMS m/z=312.1 [M+H]⁺; ¹H NMR (400 MHz, CD₃OD) δ 9.15 (s, 1H), 8.36 (d, J=4.9 Hz, 1H), 4.11 (dq, J=23.3, 8.9, 7.3 Hz, 4H), 4.02-3.75 (m, 4H), 3.43-3.33 (m, 1H), 1.44 (d, J=7.0 Hz, 9H).

### Step 2: tert-butyl 8-(2-(2-(3,4-dichlorophenyl)-2,2-difluoroacetyl)hydrazine-1-carbonyl)-6-(thiazole-5-carbonyl)-2,6-diazaspiro[3.4]octane-2-carboxylate

To a solution of 2-(tert-butoxycarbonyl)-6-(thiazole-5-carbonyl)-2,6-diazaspiro[3.4]octane-8-carboxylic acid (400 mg, 1.09 mmol) in DCM (10 mL) was added HATU (621 mg, 1.64 mmol) and DIPEA (562 mg, 4.36 mmol). The mixture was stirred at room temperature for 30 min then 2-(3,4-dichlorophenyl)-2,2-difluoroacetohydrazide (334 mg, 1.31 mmol) was added. The reaction was stirred for another 4 h then was diluted with water (30 mL), extracted with DCM (80 mL×2). The combined organic layers was washed with brine, dried over Na₂SO₄, filtered and concentrated. The residue was purified by column chromatography on silica gel (eluent: DCM:MeOH=50:1 to 20:1) to afford tert-butyl 8-(2-(2-(3,4-dichlorophenyl)-2,2-difluoroacetyl)hydrazine-1-carbonyl)-6-(thiazole-5-carbonyl)-2,6-diazaspiro[3.4]octane-2-carboxylate (300 mg, 46%) as a white solid. LCMS m/z=548.1 [M+H]⁺; ¹H NMR (400 MHz, CD₃OD) δ 9.15 (s, 1H), 8.35 (d, J=13.9 Hz, 1H), 7.87-7.80 (m, 1H), 7.71-7.64 (m, 1H), 7.64-7.57 (m, 1H), 4.25-3.79 (m, 8H), 3.28-3.20 (m, 1H), 1.44-1.40 (m, 9H).

### Step 3: tert-butyl 8-(5-((3,4-dichlorophenyl)difluoromethyl)-1,3,4-oxadiazol-2-yl)-6-(thiazole-5-carbonyl)-2,6-diazaspiro[3.4]octane-2-carboxylate

To a solution of tert-butyl 8-(2-(2-(3,4-dichlorophenyl)-2,2-difluoroacetyl)hydrazine-1-carbonyl)-6-(thiazole-5-carbonyl)-2,6-diazaspiro[3.4]octane-2-carboxylate (300 mg, 0.50 mmol) in DCM (3 mL) was added TEA (152 mg, 1.50 mmol) and TsCl (286 mg, 1.50 mmol). The reaction mixture was stirred at room temperature for 3 h then was diluted with water (20 mL), extracted with DCM (50 mL×2). The combined organic layers were washed with brine, dried over Na₂SO₄, filtered and concentrated. The residue was purified by prep-TLC (eluent: DCM:MeOH=20:1) to afford tert-butyl 8-(5-((3,4-dichlorophenyl)difluoromethyl)-1,3,4-oxadiazol-2-yl)-6-(thiazole-5-carbonyl)-2,6-diazaspiro[3.4]octane-2-carboxylate (200 mg, 66%) as a yellow solid. LCMS m/z=530.1 [M+H]⁺; ¹H NMR (400 MHz, CD₃OD) δ 9.16 (s, 1H), 8.41-8.34 (m, 1H), 7.92-7.86 (m, 1H), 7.77-7.72 (m, 1H), 7.67-7.57 (m, 1H), 4.36-3.87 (m, 8H), 1.42 (s, 9H).

### Step 4: (8-(5-((3,4-dichlorophenyl)difluoromethyl)-1,3,4-oxadiazol-2-yl)-2,6-diazaspiro[3.4]octan-6-yl)(thiazol-5-yl)methanone

To a solution of tert-butyl 8-(5-((3,4-dichlorophenyl)difluoromethyl)-1,3,4-oxadiazol-2-yl)-6-(thiazole-5-carbonyl)-2,6-diazaspiro[3.4]octane-2-carboxylate (100 mg, 0.17 mmol) in DCM (2 mL) was added TFA (0.5 mL). The reaction mixture was stirred at room temperature for 1 h then the solvent was removed under reduced pressure to afford (8-(5-((3,4-dichlorophenyl)difluoromethyl)-1,3,4-oxadiazol-2-yl)-2,6-diazaspiro[3.4]octan-6-yl)(thiazol-5-yl) methanone (99 mg, quant.) as a yellow oil. LCMS m/z=485.7 [M+H]⁺.

### Step 5: 3-(8-(5-((3,4-dichlorophenyl)difluoromethyl)-1,3,4-oxadiazol-2-yl)-6-(thiazole-5-carbonyl)-2,6-diazaspiro[3.4]octan-2-yl)-2,2-dimethyl-3-oxopropanenitrile

To a solution of 2-cyano-2-methylpropanoic acid (17 mg, 0.15 mmol) in DCM (2 mL) was added HATU (68 mg, 0.18 mmol) and DIPEA (70 mg, 0.54 mmol). The reaction was stirred at room temperature for 30 min then (8-(5-((3,4-dichlorophenyl)difluoromethyl)-1,3,4-oxadiazol-2-yl)-2,6-diazaspiro[3.4]octan-6-yl)(thiazol-5-yl)methanone (60 mg, 0.12 mmol) was added and stirring continued overnight. The reaction was diluted with water (10 mL) and extracted with DCM (30 mL×2). The combined organic layers were washed with brine, dried over Na₂SO₄, filtered and concentrated. The residue obtained was purified by prep-HPLC to afford 3-(8-(5-((3,4-dichlorophenyl)difluoromethyl)-1,3,4-oxadiazol-2-yl)-6-(thiazole-5-carbonyl)-2,6-diazaspiro[3.4]octan-2-yl)-2,2-dimethyl-3-oxopropanenitrile (9 mg, 13%) as a white solid. LCMS m/z=581.0 [M+H]⁺; ¹H NMR (400 MHz, CD₃OD) δ 9.16 (s, 1H), 8.38 (d, J=3.4 Hz, 1H), 7.89 (d, J=5.6 Hz, 1H), 7.73 (d, J=8.6 Hz, 1H), 7.66-7.59 (m, 1H), 4.77 (t, J=11.2 Hz, 1H), 4.60 (s, 1H), 4.44-3.97 (m, 7H), 1.59-1.41 (m, 6H).

### Synthesis of (2-((S)-2,2-dimethylcyclopropane-1-carbonyl)-8-(5-(1-(5-ethoxy-3-methyl-1H-pyrazol-1-yl)ethyl)-1,2,4-oxadiazol-3-yl)-2,6-diazaspiro[3.4]octan-6-yl)(thiazol-5-yl)methanone (I-42)

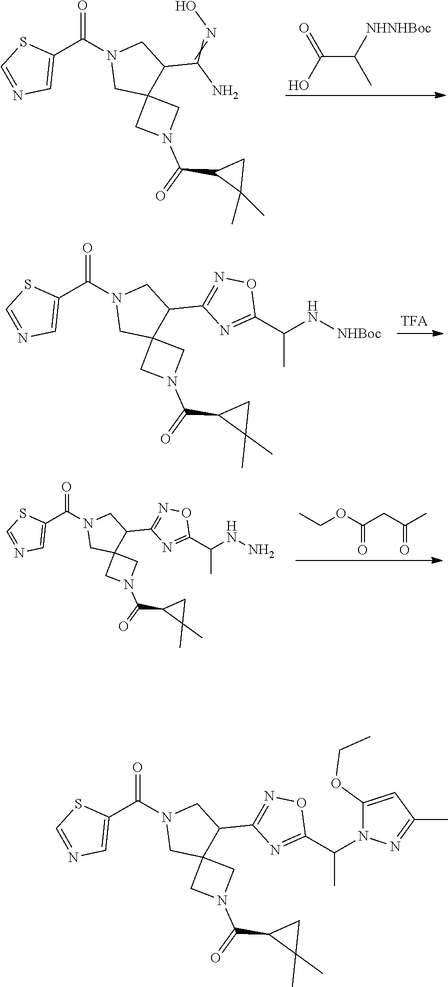

### Step 1: tert-butyl 2-(1-(3-(2-((S)-2,2-dimethylcyclopropane-1-carbonyl)-6-(thiazole-5-carbonyl)-2,6-diazaspiro[3.4]octan-8-yl)-1,2,4-oxadiazol-5-yl)ethyl)hydrazine-1-carboxylate

To a solution of ((tert-butoxycarbonyl)amino)alanine (270 mg, 0.72 mmol) in a mixture of DMF and 1,4-dioxane (3 mL and 3 mL) was added EDCI (205 mg, 1.08 mmol) and (E)-2-((S)-2,2-dimethylcyclopropane-1-carbonyl)-N′-hydroxy-6-(thiazole-5-carbonyl)-2,6-diazaspiro[3.4]octane-8-carboximidamide (220 mg, 1.08 mmol). The mixture was heated at 60° C. for 6 h then the temperature was increased to 100° C. and heating continued for 14 h. The solvent was removed under reduced pressure and the residue obtained purified by prep-TLC (DCM/MeOH=15/1) to afford tert-butyl 2-(1-(3-(2-((S)-2,2-dimethylcyclopropane-1-carbonyl)-6-(thiazole-5-carbonyl)-2,6-diazaspiro[3.4]octan-8-yl)-1,2,4-oxadiazol-5-yl)ethyl)hydrazine-1-carboxylate (200 mg, 50%) as a colorless solid. LCMS m/z=546.3 [M+H]⁺; ¹H NMR (400 MHz, CD₃OD). δ 9.17 (s, 1H), 8.48-8.36 (m, 1H), 4.50-3.79 (m, 9H), 1.43-0.99 (m, 18H), 0.94-0.83 (m, 5H), 0.82-0.70 (m, 1H).

### Step 2: (2-((S)-2,2-dimethylcyclopropane-1-carbonyl)-8-(5-(1-hydrazinylethyl)-1,2,4-oxadiazol-3-yl)-2,6-diazaspiro[3.4]octan-6-yl)(thiazol-5-yl)methanone

To a solution of tert-butyl 2-(1-(3-(2-((S)-2,2-dimethylcyclopropane-1-carbonyl)-6-(thiazole-5-carbonyl)-2,6-diazaspiro[3.4]octan-8-yl)-1,2,4-oxadiazol-5-yl)ethyl)hydrazine-1-carboxylate (50 mg, 0.09 mmol) in MeOH (2 mL) was added TFA (2 mL). The reaction mixture was stirred at room temperature for 3 h then the solvent was removed under reduced pressure to afford crude (2-((S)-2,2-dimethylcyclopropane-1-carbonyl)-8-(5-(1-hydrazinylethyl)-1,2,4-oxadiazol-3-yl)-2,6-diazaspiro[3.4]octan-6-yl)(thiazol-5-yl)methanone (40 mg, quant.) which was used in the next step without purification. LCMS m/z=446.1 [M+H]⁺.

### Step 3: (2-((S)-2,2-dimethylcyclopropane-1-carbonyl)-8-(5-(1-(5-ethoxy-3-methyl-1H-pyrazol-1-yl)ethyl)-1,2,4-oxadiazol-3-yl)-2,6-diazaspiro[3.4]octan-6-yl)(thiazol-5-yl)methanone

To a solution of (2-((S)-2,2-dimethylcyclopropane-1-carbonyl)-8-(5-(1-hydrazinylethyl)-1,2,4-oxadiazol-3-yl)-2,6-diazaspiro[3.4]octan-6-yl)(thiazol-5-yl)methanone (40 mg, 0.09 mmol) in MeOH (3.0 mL) was added ethyl 3-oxobutanoate (0.2 mL). The mixture was heated at reflux for 3 hours then the solvent removed under reduced pressure. The residue obtained was purified by prep-TLC (DCM/MeOH=15/1) to afford (2-((S)-2,2-dimethylcyclopropane-1-carbonyl)-8-(5-(1-(5-ethoxy-3-methyl-1H-pyrazol-1-yl)ethyl)-1,2,4-oxadiazol-3-yl)-2,6-diazaspiro[3.4]octan-6-yl)(thiazol-5-yl)methanone (7 mg, 15%) as a white solid. LCMS m/z=540.3 [M+H]⁺; ¹H NMR (400 MHz, CD₃OD) δ 9.16 (d, J=4.2 Hz, 1H), 8.41-8.33 (m, 1H), 5.76 (s, 1H), 5.50 (d, J=4.2 Hz, 1H), 4.59-3.83 (m, 11H), 2.13 (d, J=2.4 Hz, 2H), 1.90-1.84 (m, 2H), 1.46-1.31 (m, 6H), 1.21-1.00 (m, 7H), 0.80-0.70 (m, 1H).

### Synthesis of 1,1′-(8-(5-((3,4-dichlorophenyl)difluoromethyl)-1,3,4-oxadiazol-2-yl)-2,6-diazaspiro[3.4]octane-2,6-diyl)bis(2,2,2-trifluoroethan-1-one) (I-205)

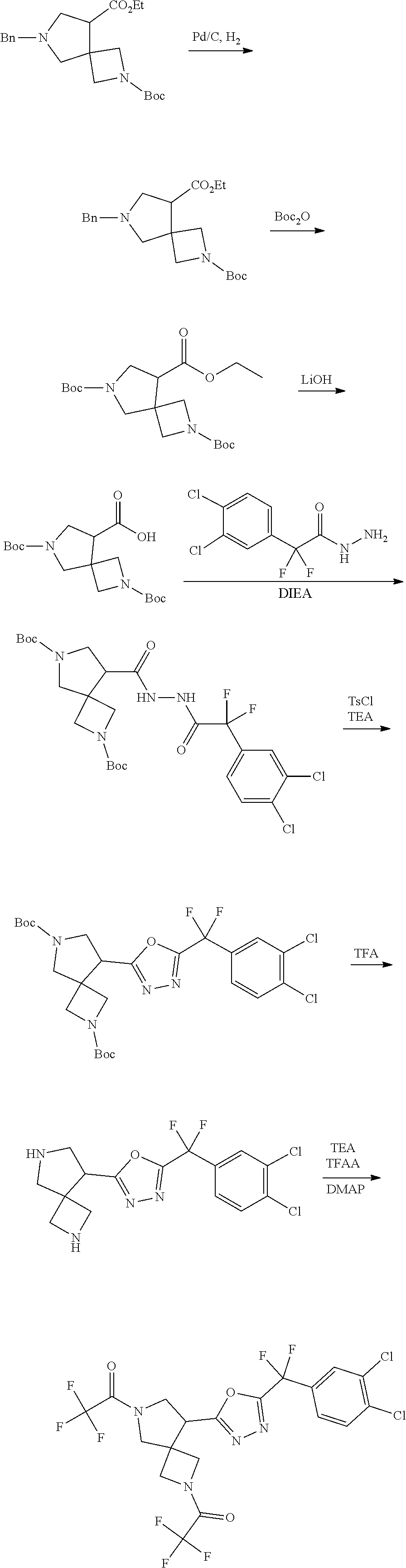

### Step 1: 2-(tert-butyl) 8-ethyl 2,6-diazaspiro[3.4]octane-2,8-dicarboxylate

To a solution of 2-(tert-butyl) 8-ethyl 6-benzyl-2,6-diazaspiro[3.4]octane-2,8-dicarboxylate (10.0 g, 26.7 mmol) in EtOAc (80 mL) was added 10% Pd/C (4.0 g). The reaction mixture was heated at 55° C. under H₂ atmosphere for 14 h. The catalyst was removed by filtration through celite and the filterate concentrated to afford crude 2-(tert-butyl) 8-ethyl 2,6-diazaspiro[3.4]octane-2,8-dicarboxylate (7.0 g, 92%) as yellow oil which was used in the next step without purification. LCMS m/z=285.2 [M+H]⁺; ¹H NMR (400 MHz, DMSO-d₆) δ 4.19-4.01 (m, 2H), 3.84 (d, J=8.0 Hz, 1H), 3.76-3.65 (m, 2H), 3.61 (q, J=7.6, 5.4 Hz, 1H), 3.09-3.01 (m, 1H), 3.01-2.84 (m, 4H), 1.36 (s, 9H), 1.19 (t, J=7.2 Hz, 3H).

### Step 2: 2,6-di-tert-butyl 8-ethyl 2,6-diazaspiro[3.4]octane-2,6,8-tricarboxylate

To a solution of 2-(tert-butyl) 8-ethyl 2,6-diazaspiro[3.4]octane-2,8-dicarboxylate (3.8 g, 13.36 mmol) in DCM (20 mL) was added TEA (2.7 g, 26.73 mmol) and (BOC)₂O (3.2 g, 14.70 mmol). The reaction was stirred at room temperature overnight then diluted with water (100 mL) and extracted with DCM (100 mL×3). The combined organic layers were washed with brine, dried over Na₂SO₄, filtered and concentrated to afford crude 2,6-di-tert-butyl 8-ethyl 2,6-diazaspiro[3.4]octane-2,6,8-tricarboxylate (4.9 g, 95%) as yellow oil which was used directly in the next step. LCMS m/z=407.2 [M+Na]⁺.

### Step 3: 2,6-bis(tert-butoxycarbonyl)-2,6-diazaspiro[3.4]octane-8-carboxylic acid

To a solution of 2,6-di-tert-butyl 8-ethyl 2,6-diazaspiro[3.4]octane-2,6,8-tricarboxylate (2.1 g, 5.46 mmol) in a mixture of THF and water (16 mL/4 mL) was added LiOH (261.6 mg, 10.92 mmol). The reaction was stirred at room temperature overnight then was diluted with 1 M HCl (50 mL) and extracted with EtOAc (100×2 mL). The combined organic layers were washed with brine, dried over Na₂SO₄, filtered and concentrated to afford 2,6-bis(tert-butoxycarbonyl)-2,6-diazaspiro[3.4]octane-8-carboxylic acid (1.5 g, 77%) as yellow oil which was used in the next step without purification. LCMS m/z=355.2 [M−H]⁻; ¹H NMR (400 MHz, DMSO-d₆) δ 12.79 (s, 1H), 3.76 (s, 4H), 3.56-3.39 (m, 4H), 3.17 (d, J=8.0 Hz, 1H), 1.40 (d, J=8.0 Hz, 18H).

### Step 4: di-tert-butyl 8-(2-(2-(3,4-dichlorophenyl)-2,2-difluoroacetyl)hydrazine-1-carbonyl)-2,6-diazaspiro[3.4]octane-2,6-dicarboxylate

To a solution of 2-(3,4-dichlorophenyl)-2,2-difluoroacetohydrazide (1.2 g, 4.63 mmol) in DMF (40 mL) was added 2,6-bis(tert-butoxycarbonyl)-2,6-diazaspiro[3.4]octane-8-carboxylic acid (1.5 g, 4.21 mmol), EDCI (1.2 g, 6.31 mmol), HOBt (853 g, 6.31 mmol) and DIPEA (1.6 g, 12.63 mmol). The reaction was stirred at room temperature under a nitrogen atmosphere overnight then was diluted with water (70 mL) and extracted with EtOAc (100 mL×3). The combined organic layers were washed with brine, dried over Na₂SO₄, filtered and concentrated to afford the crude di-tert-butyl 8-(2-(2-(3,4-dichlorophenyl)-2,2-difluoroacetyl)hydrazine-1-carbonyl)-2,6-diazaspiro[3.4]octane-2,6-dicarboxylate (2.7 g) as yellow oil which was used directly in the next step. LCMS m/z=393.1 [M+H−200]⁺; ¹H NMR (400 MHz, DMSO-d₆) δ 7.86-7.43 (m, 3H), 3.97-3.42 (m, 6H), 2.81 (d, J=63.2 Hz, 1H), 2.11 (d, J=5.6 Hz, 2H), 1.37 (d, J=14.8 Hz, 18H).

### Step 5: di-tert-butyl 8-(5-((3,4-dichlorophenyl)difluoromethyl)-1,3,4-oxadiazol-2-yl)-2,6-diazaspiro[3.4]octane-2,6-dicarboxylate

To a solution of di-tert-butyl 8-(2-(2-(3,4-dichlorophenyl)-2,2-difluoroacetyl)hydrazine-1-carbonyl)-2,6-diazaspiro[3.4]octane-2,6-dicarboxylate (2.7 g, 4.55 mmol) in DCM (30 mL) was added TEA (1.4 g, 12.63 mmol) and TsCl (2.6 g, 6.31 mmol). The reaction was stirred at room temperature overnight then was diluted with water (60 mL), extracted with EtOAc (80 mL×3). The combined organic layers were washed with brine, dried over Na₂SO₄, filtered and concentrated. The residue was purified by column chromatography on silica gel (eluent: Pet. Ether:EtOAc=2:1) to afford di-tert-butyl 8-(5-((3,4-dichlorophenyl)difluoromethyl)-1,3,4-oxadiazol-2-yl)-2,6-diazaspiro[3.4]octane-2,6-dicarboxylate (1.8 g, 70%) as yellow oil. LCMS m/z=597.2 [M+Na]⁺; ¹H NMR (400 MHz, DMSO-d₆) δ 8.02 (d, J=2.0 Hz, 1H), 7.89 (d, J=8.4 Hz, 1H), 7.72 (dd, J=8.4, 2.0 Hz, 1H), 4.08-3.51 (m, 9H), 1.46-1.30 (m, 18H).

### Step 6: 2-((3,4-dichlorophenyl)difluoromethyl)-5-(2,6-diazaspiro[3.4]octan-8-yl)-1,3,4-oxadiazole

To a solution of 8-(5-((3,4-dichlorophenyl)difluoromethyl)-1,3,4-oxadiazol-2-yl)-2,6-diazaspiro[3.4]octane-2,6-dicarboxylate (200 mg, 0.34 mmol) in DCM (2 mL) was added TFA (1 mL). The reaction stirred at room temperature for 2 h then the solvent was removed under reduced pressure to afford crude 2-((3,4-dichlorophenyl)difluoromethyl)-5-(2,6-diazaspiro[3.4]octan-8-yl)-1,3,4-oxadiazole (130 mg, 99%) which was used directly in next step. LCMS m/z=375.05 [M+H−200]⁺.

### Step 7: 1,1′-(8-(5-((3,4-dichlorophenyl)difluoromethyl)-1,3,4-oxadiazol-2-yl)-2,6-diazaspiro[3.4]octane-2,6-diyl)bis(2,2,2-trifluoroethan-1-one)

To a solution of 2-((3,4-dichlorophenyl)difluoromethyl)-5-(2,6-diazaspiro[3.4]octan-8-yl)-1,3,4-oxadiazole (130 mg, 0.66 mmol) in DCM (4 mL) was added TEA (420 mg, 8.0 mmol), DMAP (8.47 mg, 0.13 mmol) and TFAA (436 mg, 4.0 mmol). The reaction was stirred at room temperature under N₂ atmosphere overnight then was diluted with water (30 mL) and extracted with DCM (50 mL×3). The combined organic layers were washed with brine, dried over Na₂SO₄, filtered and concentrated. The residue was purified by prep-HPLC to afford the 1,1′-(8-(5-((3,4-dichlorophenyl)difluoromethyl)-1,3,4-oxadiazol-2-yl)-2,6-diazaspiro[3.4]octane-2,6-diyl)bis(2,2,2-trifluoroethan-1-one) (70 mg, 35%) as a white solid. LCMS m/z=624.4 [M+H]⁺; ¹H NMR (400 MHz, Methanol-d⁴) δ 7.87 (d, J=2.8 Hz, 1H), 7.75-7.71 (m, 1H), 7.62 (s, 1H), 4.65-4.54 (m, 1H), 4.47 (s, 1H), 4.32-4.14 (m, 5H), 4.13-3.96 (m, 2H).

### Synthesis of Building blocks: 2-(3,4-dichlorophenyl)acetohydrazide

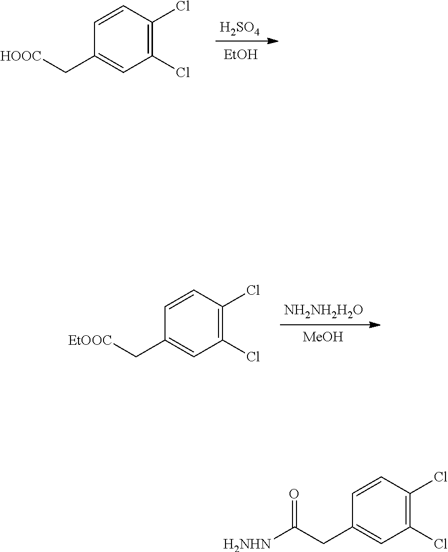

### Step 1: ethyl 2-(3,4-dichlorophenyl)acetate

To a solution of 2-(3,4-dichlorophenyl)acetic acid (10 g, 49 mmol) in EtOH (100 mL) was added H₂SO₄ (4.8 g, 49 mmol). The reaction was heated at 40° C. overnight then was diluted with water (50 mL) and extracted with EtOAc (100 mL×3). The combined organic layers were washed with brine, dried over Na₂SO₄, filtered and concentrated. The residue obtained was purified by column chromatography on silica gel (eluent: Pet. Ether:EtOAc=100:1 to 50:1) to afford ethyl 2-(3,4-dichlorophenyl)acetate (10 g, 92%) as a colorless oil. ¹H NMR (400 MHz, DMSO-d₆) δ 7.58-7.52 (m, 2H), 7.28-7.23 (m, 1H), 4.08 (q, J=7.1 Hz, 2H), 3.71 (s, 2H), 1.17 (t, J=7.1 Hz, 3H).

### Step 2: 2-(3,4-dichlorophenyl)acetohydrazide

To a solution of ethyl 2-(3,4-dichlorophenyl)acetate (1 g, 4 mmol) in MeOH (10 mL) under a nitrogen atmosphere was added hydrazine hydrate (220 mg, 4.4 mmol). The reaction mixture was heated at 80° C. for 3 h then the solvent was removed under reduced pressure to afford 2-(3,4-dichlorophenyl)acetohydrazide (690 mg, 80%) which was used in the next step without purification. LCMS n z=219.0 [M+H]⁺.

### Synthesis of 2-(6-(tetrahydro-2H-pyran-4-yl)pyridin-2-yl)acetohydrazide

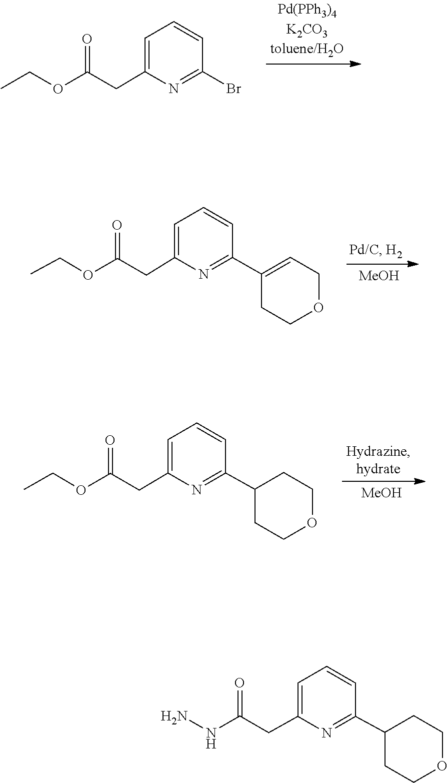

### Step 1: ethyl 2-(6-(3,6-dihydro-2H-pyran-4-yl)pyridin-2-yl)acetate

To a solution of ethyl 2-(6-bromopyridin-2-yl)acetate (1 g, 4.1 mmol) in a mixture of toluene (10 mL) and water (2 mL) was added 2-(3,4-dihydro-2H-pyran-5-yl)-4,4,5,5-tetramethyl-1,3,2-dioxaborolane (1.12 g, 5.3 mmol), K₂CO₃ (1.13 g, 8.2 mmol) and Pd(PPh₃)₄ (473 mg, 0.41 mmol). The reaction was heated at 100° C. under N₂ atmosphere overnight then was diluted with water (50 mL) and extracted with EtOAc (100 mL×3). The combined organic layers were dried over Na₂SO₄, filtered and concentrated. The residue was purified by RP-column to afford ethyl 2-(6-(3,6-dihydro-2H-pyran-4-yl)pyridin-2-yl)acetate (917 mg, 91% yield) as a yellow oil. LCMS m/z=248.1[M+H]⁺; ¹H NMR (400 MHz, DMSO-d₆) δ 7.94-7.87 (m, 1H), 7.56-7.51 (m, 1H), 7.38-7.33 (m, 1H), 6.81-6.78 (m, 1H), 4.29-4.26 (m, 2H), 4.14-4.09 (m, 3H), 3.95-3.93 (m, 2H), 3.85-3.80 (m, 3H), 1.20-1.19 (m, 3H).

### Step 2: ethyl 2-(6-(tetrahydro-2H-pyran-4-yl)pyridin-2-yl)acetate

To a solution of ethyl 2-(6-(3,6-dihydro-2H-pyran-4-yl)pyridin-2-yl)acetate (910 mg, 3.68 mmol) in MeOH (7 mL) was added 10% Pd/C (273 mg). The reaction was stirred overnight at room temperature under a H₂ atmosphere. The catalyst was removed by filtration through celite and the filtrate concentrated to afford ethyl 2-(6-(tetrahydro-2H-pyran-4-yl)pyridin-2-yl)acetate (970 mg, 100% yield) as a yellow oil, which was without purification. LCMS m/z=250.1[M+H]⁺; ¹H NMR (400 MHz, DMSO-d₆) δ 8.27-8.08 (m, 1H), 7.86-7.48 (m, 1H), 4.13-4.10 (m, 2H), 4.02-3.94 (m, 2H), 3.46-3.40 (m, 1H), 1.84-1.78 (m, 3H), 1.53-1.39 (m, 1H), 1.20-1.17 (m, 3H).

### Step 3: 2-(6-(tetrahydro-2H-pyran-4-yl)pyridin-2-yl)acetohydrazide

To a solution of ethyl 2-(6-(tetrahydro-2H-pyran-4-yl)pyridin-2-yl)acetate (200 mg, 0.80 mmol) in MeOH (1.5 mL) at 0° C. was added NH₂NH₂ (98%, 12 drops). The reaction was heated at 80° C. for 2.5 h then concentrated under reduced pressure. The residue obtained was purified by prep-TLC (DCM/MeOH=15/1, v/v) to afford crude 2-(6-(tetrahydro-2H-pyran-4-yl)pyridin-2-yl)acetohydrazide (68 mg, 36%) as a colorless oil. LCMS m/z=236.1[M+H]⁺; ¹H NMR (400 MHz, Chloroform-d) δ 8.64 (s, 1H), 7.63-7.57 (m, 1H), 7.11-7.06 (m, 2H), 4.12-4.07 (m, 2H), 3.74-3.70 (m, 2H), 3.58-3.51 (m, 2H), 1.88-1.82 (m, 4H), 1.32-1.19 (m, 2H), 0.91-0.77 (m, 1H).

### Synthesis of 2-(3-fluoro-6-(tetrahydro-2H-pyran-4-yl)pyridin-2-yl)acetohydrazide

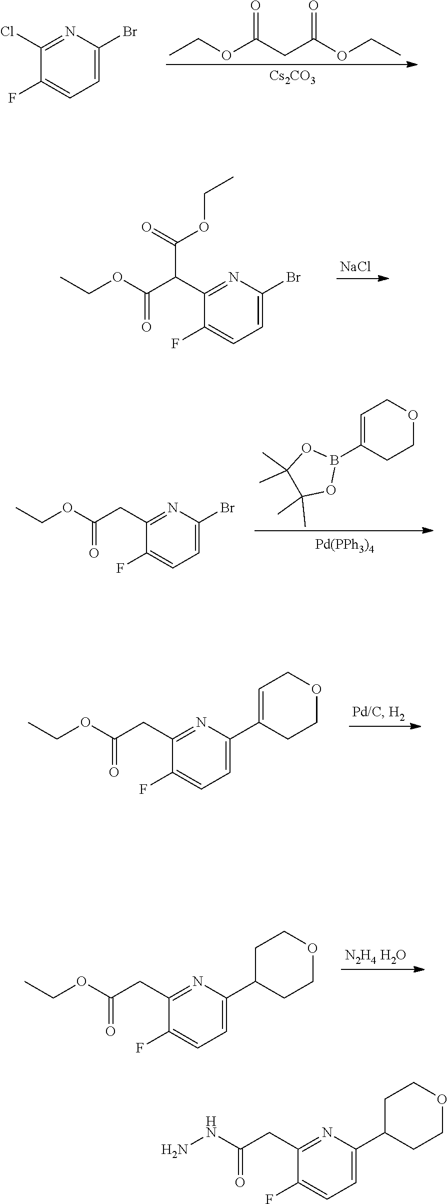

### Step 1: diethyl 2-(6-bromo-3-fluoropyridin-2-yl)malonate

To a solution of 6-bromo-2-chloro-3-fluoropyridine (500 mg, 2.38 mmol) and Cs₂CO₃ (1.55 g, 4.75 mmol) in DMSO (5 mL) at room temperature was added diethyl malonate (571 mg, 3.56 mmol). The reaction mixture was heated at 100° C. for 1 h then the solvent was removed under reduced pressure. The residue obtained was purified by RP-column to afford diethyl 2-(6-bromo-3-fluoropyridin-2-yl)malonate (220 mg, 27%) as a yellow solid. LCMS m/z=581.3 [M+H]⁺; ¹H NMR (400 MHz, DMSO-d₆) δ 9.31-9.22 (m, 1H), 8.43-8.34 (m, 1H), 7.71-7.61 (m, 1H), 7.34-7.28 (m, 1H), 4.54-4.41 (m, 2H), 4.34-4.04 (m, 5H), 3.99-3.79 (m, 6H), 3.41 (s, 2H), 2.92-2.82 (m, 1H), 1.72-1.60 (m, 4H), 1.36-1.23 (m, 1H), 1.12-0.94 (m, 6H), 0.89-0.81 (m, 1H), 0.73-0.60 (m, 1H).

### Step 2: ethyl 2-(6-bromo-3-fluoropyridin-2-yl)acetate

To a solution of diethyl 2-(6-bromo-3-fluoropyridin-2-yl)malonate (100 mg, 0.30 mmol) in a mixture of DMSO and water (2 mL/1 mL) at room temperature was added NaCl (53 mg, 0.90 mmol). The reaction mixture was heated at 150° C. for 6 h then diluted with water (10 mL) and extracted with EtOAc (20 mL×3). The combined organic layers were washed with brine, dried over Na₂SO₄, filtered and concentrated. The residue obtained was purified by prep-TLC (eluent: Pet. Ether/EtOAc=15/1, v/v) to afford ethyl 2-(6-bromo-3-fluoropyridin-2-yl)acetate (40 mg, 51%) as a colorless oil. LCMS m/z=261.0 [M+H]⁺; ¹H NMR (400 MHz, DMSO-d₆) δ 7.75 (t, J=8.8 Hz, 1H), 7.67 (dd, J=8.6, 3.6 Hz, 1H), 4.12 (q, J=7.1 Hz, 2H), 3.90 (d, J=2.5 Hz, 2H), 1.18 (t, J=7.1 Hz, 3H).

### Step 3: ethyl 2-(6-(3,6-dihydro-2H-pyran-4-yl)-3-fluoropyridin-2-yl)acetate

To a solution of ethyl 2-(6-bromo-3-fluoropyridin-2-yl)acetate (80 mg, 0.31 mmol) in a mixture of toluene (2 mL) and water (1 mL) was added 2-(3,6-dihydro-2H-pyran-4-yl)-4,4,5,5-tetramethyl-1,3,2-dioxaborolane (64 mg, 0.31 mmol), K₂CO₃ (85 mg, 0.61 mmol) and Pd(PPh₃)₄ (18 mg, 0.015 mmol). The reaction was heated at 100° C. under N₂ atmosphere overnight then diluted with water (20 mL) and extracted with EtOAc (50 mL×2). The combined organic layers were dried over Na₂SO₄, filtered and concentrated. The residue was purified by prep-TLC (eluent: Pet. Ether/EtOAc=10/1, v/v) to afford ethyl 2-(6-(3,6-dihydro-2H-pyran-4-yl)-3-fluoropyridin-2-yl)acetate (10 mg, 12%) as a colorless oil. LCMS m/z=266.1 [M+H]⁺; ¹H NMR (400 MHz, Chloroform-d) δ 7.40-7.34 (m, 1H), 7.32-7.27 (m, 1H), 6.64 (s, 1H), 4.35 (q, J=2.9 Hz, 2H), 4.20 (q, J=7.1 Hz, 2H), 3.95-3.90 (m, 4H), 2.63-2.56 (m, 2H), 1.27 (s, 3H).

### Step 4: ethyl 2-(3-fluoro-6-(tetrahydro-2H-pyran-4-yl)pyridin-2-yl)acetate

To a solution of ethyl 2-(6-(3,6-dihydro-2H-pyran-4-yl)-3-fluoropyridin-2-yl)acetate (57 mg, 0.21 mmol) in MeOH (8 mL) was added 10% Pd/C (20 mg). The reaction was stirred at room temperature under a H₂ atmosphere overnight. The catalyst was removed by filtration through celite then the filtrate was concentrated to afford the ethyl 2-(3-fluoro-6-(tetrahydro-2H-pyran-4-yl)pyridin-2-yl)acetate (57 mg, 100% yield) as a colorless oil, which was used without further purification. LCMS m/z=268.1 [M+H]⁺.

### Step 5: 2-(3-fluoro-6-(tetrahydro-2H-pyran-4-yl)pyridin-2-yl)acetohydrazide

To a solution of ethyl 2-(3-fluoro-6-(tetrahydro-2H-pyran-4-yl)pyridin-2-yl)acetate (57 mg, 0.21 mmol) in MeOH (1 mL) was added 98% hydrazine hydrate (4 drops). The reaction was stirred at room temperature overnight then concentrated under reduced pressure to afford 2-(3-fluoro-6-(tetrahydro-2H-pyran-4-yl)pyridin-2-yl)acetohydrazide (44 mg crude, 81% yield) as a white solid which was used without further purification. LCMS m/z=254.2 [M+H]⁺.

### Synthesis of 2-(3-methoxy-6-(tetrahydro-2H-pyran-4-yl)pyridin-2-yl)acetohydrazide

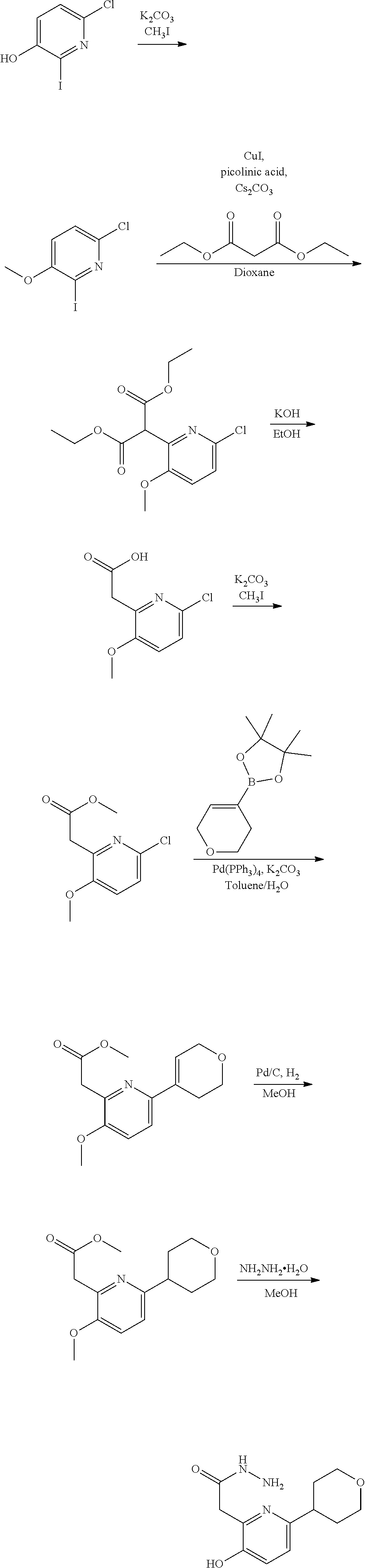

### Step 1: 6-chloro-2-iodo-3-methoxypyridine

To a mixture of 6-chloro-2-iodopyridin-3-ol (2.00 g, 0.01 mmol) and K₂CO₃ (3.20 g, 0.02 mmol) in DMF (8 mL) in a sealed tube was added CH₃I (3.31 g, 0.02 mmol). The reaction was stirred at room temperature overnight then diluted with water (40 mL) and extracted with EtOAc (100 mL×2). The combined organic layers were washed with brine, dried over Na₂SO₄, filtered and concentrated. The residue obtained was purified by column chromatography on silica gel (eluent: Pet. Ether:EtOAc=15:1) to afford 6-chloro-2-iodo-3-methoxypyridine (1.80 g, 90%) as a white solid. LCMS m/z=268.9 [M+H]⁺; ¹H NMR (DMSO, 400 MHz) δ7.49 (JH, d, J=8.6 Hz), 7.42 (JH, d, J=8.6 Hz), 3.88 (3H, s).

### Step 2: diethyl 2-(6-chloro-3-methoxypyridin-2-yl)malonate

To a solution of 6-chloro-2-iodo-3-methoxypyridine (1.50 g, 5.58 mmol), CuI (106 mg, 0.56 mmol), picolinic acid (137 mg, 1.12 mmol) and Cs₂CO₃ (5.45 g, 16.73 mmol) in dioxane (20 mL) under a N₂ atmosphere was added diethyl malonate (1.79 g, 11.16 mmol). The mixture was heated at reflux for 5 h then diluted with water (30 mL) and extracted with EtOAc (100 mL×2). The combined organic layers were washed with brine, dried over Na₂SO₄, filtered and concentrated. The residue obtained was purified by column chromatography on silica gel (eluent: Pet. Ether:EtOAc=15:1 to 5:1) to afford diethyl 2-(6-chloro-3-methoxypyridin-2-yl)malonate (856 mg, 51%) as a white solid. LCMS m/z=301.1 [M+H]⁺; ¹H NMR (DMSO, 400 MHz) δ 7.59 (1H, d, J=8.8 Hz), 4.11 (4H, d, J=7.2 Hz), 3.83 (2H, s), 3.47 (1H, s), 1.19 (6H, s).

### Step 3: 2-(6-chloro-3-methoxypyridin-2-yl)acetic acid

To a solution of 2-(6-chloro-3-methoxypyridin-2-yl)malonate (800 mg, 2.66 mmol) in EtOH (8 mL) was added KOH (745 mg, 13.29 mmol). The reaction heated at 85° C. for 5 h then was diluted with water (30 mL), and extracted with EtOAc (30 mL). The aqueous layer was collected and acidified to pH˜2 with 1M HCl then extracted with EtOAc (100 mL×2). The combined organic layers were washed with brine, dried over Na₂SO₄, filtered and concentrated. The residue obtained was purified by prep-TLC (eluent: Pet. Ether:EtOAc=5:1) to afford 2-(6-chloro-3-methoxypyridin-2-yl)acetic acid (315 mg, 60%) as a yellow oil. LCMS m/z=201.0 [M+H]⁺.

### Step 4: methyl 2-(6-chloro-3-methoxypyridin-2-yl)acetate

To a solution of 2-(6-chloro-3-methoxypyridin-2-yl)acetic acid (300 mg, 1.49 mmol) and K₂CO₃ (619 mg, 4.48 mmol) in DMF (3 mL) in a sealed tube was added CH₃I (424 mg, 2.98 mmol). The reaction was stirred at room temperature overnight then was diluted with water (30 mL) and extracted with EtOAc (80 mL×2). The combined organic layers were washed with brine, dried over Na₂SO₄, filtered and concentrated to afford methyl 2-(6-chloro-3-methoxypyridin-2-yl)acetate (315 mg, 103%) as a yellow oil which was used without further purification. LCMS m/z=215.0 [M+H]⁺; ¹H NMR (400 MHz, DMSO-d₆) δ 7.53 (d, J=8.4 Hz, 1H), 7.41 (d, J=8.8 Hz, 1H), 3.81 (s, 3H), 3.76 (s, 2H), 3.61 (s, 3H).

### Step 5: methyl 2-(6-(3,6-dihydro-2H-pyran-4-yl)-3-methoxypyridin-2-yl)acetate

To a solution of methyl 2-(6-chloro-3-methoxypyridin-2-yl)acetate (310 mg, 1.4 mmol), 2-(3,6-dihydro-2H-pyran-4-yl)-4,4,5,5-tetramethyl-1,3,2-dioxaborolane (363 mg, 1.7 mmol) and K₂CO₃ (598 mg, 4.3 mmol) in a mixture of toluene and water (2 mL/0.4 mL) was added Pd(PPh₃)₄ (188 mg, 0.2 mmol). The reaction mixture was heated at 100° C. for 2 h then was diluted with water (30 mL) and extracted with EtOAc (50 mL×2). The combined organic layers were washed with brine, dried over Na₂SO₄, filtered and concentrated. The residue was purified by column chromatography on silica gel (eluent: Pet. Ether:EtOAc=10:1 to 5:1) to afford methyl 2-(6-(3,6-dihydro-2H-pyran-4-yl)-3-methoxypyridin-2-yl)acetate (150 mg, 40%) as a yellow solid. LCMS m/z=263.1 [M+H]⁺; ¹H NMR (DMSO, 400 MHz) δ 7.41 (2H, d, J=3.4 Hz), 6.58 (1H, s), 4.23 (2H, d, J=2.8 Hz), 3.78-3.82 (5H, m), 3.76 (2H, s), 3.61 (3H, d, J=0.8 Hz), 2.47 (2H, s).

### Step 6: methyl 2-(3-methoxy-6-(tetrahydro-2H-pyran-4-yl)pyridin-2-yl)acetate

To a solution of methyl 2-(6-(3,6-dihydro-2H-pyran-4-yl)-3-methoxypyridin-2-yl)acetate (170 mg, 0.6 mmol) in MeOH (4 mL) was added 10% Pd/C (68 mg). The reaction mixture was stirred under a H₂ atmosphere for 4 h. The catalyst was removed by filtration through celite and the filtrate concentrated to afford methyl 2-(3-methoxy-6-(tetrahydro-2H-pyran-4-yl)pyridin-2-yl)acetate (164 mg, 96%) as a yellow oil. LCMS m/z=265.1 [M+H]⁺; ¹H NMR (CDCl₃, 400 MHz) δ 7.11 (1H, d, J=8.8 Hz), 7.04 (1H, d, J=8.4 Hz), 4.02-4.09 (2H, m), 3.85 (2H, s), 3.80 (3H, s), 3.70 (3H, s), 3.52 (2H, td, J=11.2, 3.4 Hz), 2.89 (1H, t, J=5.2 Hz), 1.83 (4H, q, J=4.6 Hz).

### Step 7: 2-(3-methoxy-6-(tetrahydro-2H-pyran-4-yl)pyridin-2-yl)acetohydrazide

To a solution of methyl 2-(3-methoxy-6-(tetrahydro-2H-pyran-4-yl)pyridin-2-yl)acetate (150 mg, 0.6 mmol) in MeOH (2 mL) was added 98% hydrazine hydrate (0.8 mL). The reaction mixture was stirred at room temperature for 1.5 h then was diluted with water (30 mL) and extracted with EtOAc (80 mL×2). The combined organic layers were washed with brine, dried over Na₂SO₄, filtered and concentrated. The residue obtained was purified by prep-TLC (eluent: Pet. Ether:EtOAc=2:1) to afford 2-(3-methoxy-6-(tetrahydro-2H-pyran-4-yl)pyridin-2-yl)acetohydrazide (120 mg, 80%) as a white solid. LCMS m/z=265.14 [M+H]⁺; ¹H NMR (CDCl₃, 400 MHz) δ 7.10 (1H, s), 7.05 (1H, s), 4.06 (2H, d, J=10.0 Hz), 3.85 (2H, s), 3.80 (3H, s), 3.69 (10H, s), 3.52 (2H, d, J=3.2 Hz), 2.89 (1H, tt, J=10.2, 4.8 Hz), 1.78-1.87 (4H, m).

### Synthesis of 3-(6-(tetrahydro-2H-pyran-4-yl)pyridin-2-yl)oxetane-3-carbohydrazide

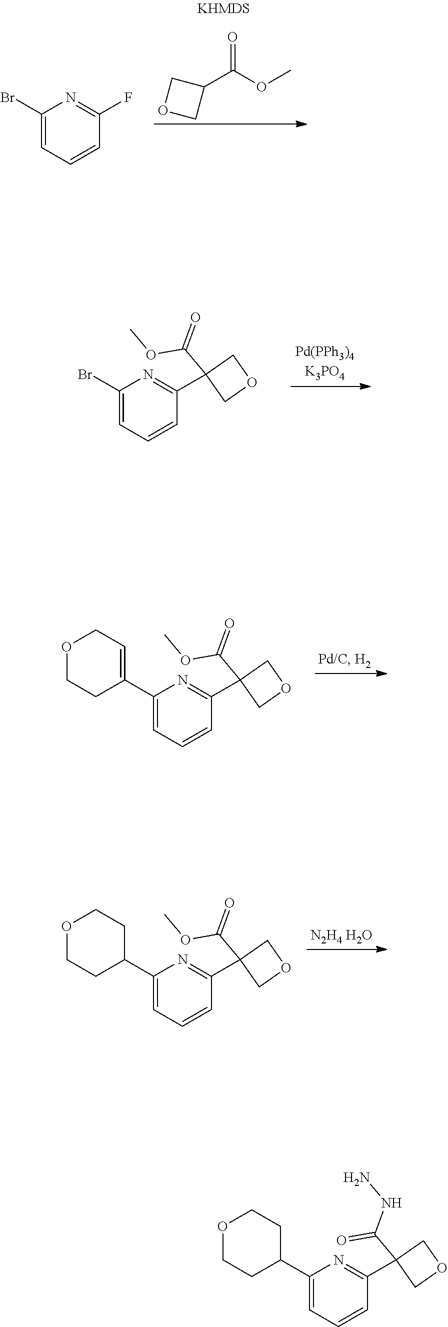

### Step 1: methyl 3-(6-bromopyridin-2-yl)oxetane-3-carboxylate

To a solution of 2-bromo-6-fluoropyridine (300 mg, 1.70 mmol) and methyl oxetane-3-carboxylate in toluene (3 mL) under N₂ atmosphere at 0° C. was added KHMDS (2.3 mL, 1.0 M in THF, 2.22 mmol). The mixture was stirred at room temperature overnight then diluted with saturated aq. NH₄Cl (30 mL) and extracted with EtOAc (50 mL×3). The combined organic layers were washed with brine, dried over Na₂SO₄, filtered and concentrated. The residue was purified by prep-TLC (eluent: Pet. Ether/EtOAc=5/1, v/v) to afford methyl 3-(6-bromopyridin-2-yl)oxetane-3-carboxylate (60 mg, 12%) as a colorless oil. ¹H NMR (400 MHz, DMSO-d₆) δ 7.82 (t, J=7.8 Hz, 1H), 7.63 (d, J=7.8 Hz, 1H), 7.52 (d, J=7.6 Hz, 1H), 5.04 (d, J=6.2 Hz, 2H), 4.93 (d, J=6.4 Hz, 2H), 4.72-4.68 (m, 1H), 4.61 (t, J=6.2 Hz, 1H), 3.70 (s, 3H).

### Step 2: methyl 3-(6-(3,6-dihydro-2H-pyran-4-yl)pyridin-2-yl)oxetane-3-carboxylate

To a solution of methyl 3-(6-bromopyridin-2-yl)oxetane-3-carboxylate (60 mg, 0.22 mmol) in dioxane (2 mL) was added 2-(3,6-dihydro-2H-pyran-4-yl)-4,4,5,5-tetramethyl-1,3,2-dioxaborolane (69 mg, 0.33 mmol), K₃PO₄ (94 mg, 0.44 mmol) and Pd(PPh₃)₄ (26 mg, 0.022 mmol). The reaction was heated at 100° C. under N₂ atmosphere overnight then was diluted with water (20 mL) and extracted with EtOAc (50 mL×2). The combined organic layers were dried over Na₂SO₄, filtered and concentrated. The residue was purified by prep-TLC (eluent: Pet. Ether/EtOAc=5/1, v/v) to afford methyl 3-(6-(3,6-dihydro-2H-pyran-4-yl)pyridin-2-yl)oxetane-3-carboxylate (54 mg, 90%) as a colorless oil. LCMS m/z=276.7 [M+H]⁺; ¹H NMR (400 MHz, DMSO-d₆) δ 7.85 (t, J=7.8 Hz, 1H), 7.52-7.49 (m, 1H), 7.35 (d, J=7.8 Hz, 1H), 6.82-6.77 (m, 1H), 5.06 (d, J=6.2 Hz, 2H), 4.99 (d, J=6.2 Hz, 2H), 4.27 (q, J=2.8 Hz, 2H), 3.82 (t, J=5.6 Hz, 2H), 3.70 (s, 3H), 2.53 (d, J=2.8 Hz, 2H).

### Step 3: methyl 3-(6-(tetrahydro-2H-pyran-4-yl)pyridin-2-yl)oxetane-3-carboxylate

To a solution of methyl 3-(6-(3,6-dihydro-2H-pyran-4-yl)pyridin-2-yl)oxetane-3-carboxylate (80 mg, 0.29 mmol) in MeOH (1 mL) was added 10% Pd/C (32 mg). The reaction was stirred at room temperature under a H₂ atmosphere overnight. The catalyst was removed by filtration through celite and concentrated to afford the methyl 3-(6-(tetrahydro-2H-pyran-4-yl)pyridin-2-yl)oxetane-3-carboxylate (64 mg, 80%) as a colorless oil, which was used in the next step without purification. LCMS m/z=278.1 [M+H]⁺.

### Step 4: 3-(6-(tetrahydro-2H-pyran-4-yl)pyridin-2-yl)oxetane-3-carbohydrazide

To a solution of methyl 3-(6-(tetrahydro-2H-pyran-4-yl)pyridin-2-yl)oxetane-3-carboxylate (64 mg, 0.23 mmol) in MeOH (1 mL) was added 98% hydrazine hydrate (6 drops). The reaction was stirred at room temperature overnight then was concentrated under reduced pressure. The residue was purified by prep-TLC (eluent: DCM/MeOH=5/1, v/v) to afford 3-(6-(tetrahydro-2H-pyran-4-yl)pyridin-2-yl)oxetane-3-carbohydrazide (35 mg, 54% yield) as a yellow solid. LCMS m/z=277.5 [M+H]⁺.

### Synthesis of 2-(6-(tetrahydro-2H-pyran-2-yl)pyridin-2-yl)acetohydrazide

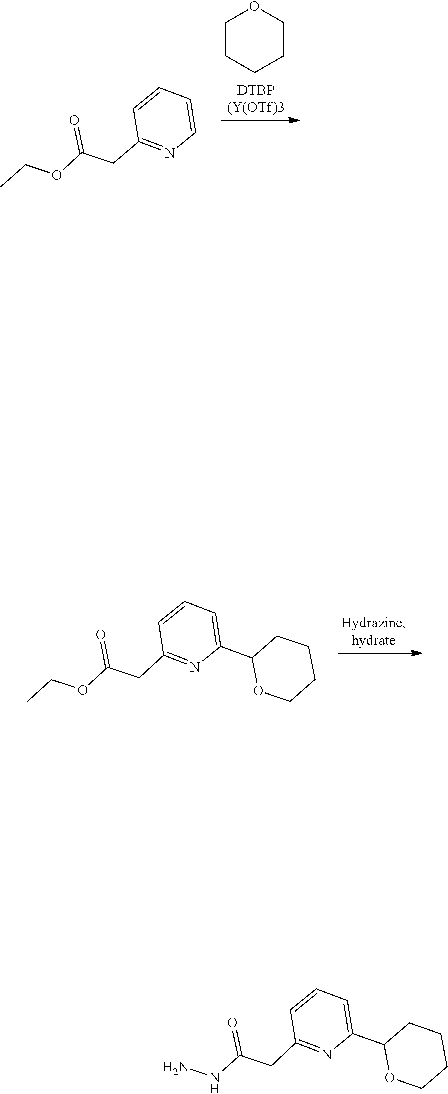 [SMILES: C1CCOCC1 ; CCOC(=O)Cc1ccccn1 ; CCOC(=O)Cc1cccc(C2CCCCO2)n1 ; NNC(=O)Cc1cccc(C2CCCCO2)n1]

### Step 1: ethyl 2-(6-(tetrahydro-2H-pyran-2-yl)pyridin-2-yl)acetate

To a solution of ethyl 2-(pyridin-2-yl)acetate (5 g, 0.03 mol) in bis(tert-butyl)peroxide (13.1 g, 0.09 mol) was added tetrahydro-2H-pyran (2.6 g, 0.03 mol) and Y(OTf)3 (40 mg, 0.03 mmol). The reaction mixture was heated at 120° C. for 48 h then the solvent was removed under reduced pressure. The residue obtained was purified by column chromatography on silica gel (eluent: Pet. Ether:EtOAc=20:1) to afford ethyl 2-(6-(tetrahydro-2H-pyran-2-yl)pyridin-2-yl)acetate (200 mg, 3%) as a yellow oil. LCMS m/z=250.2 [M+H]⁺; ¹H NMR (400 MHz, DMSO-d6) δ 7.74 (t, J=7.6 Hz, 1H), 7.30 (d, J=7.8 Hz, 1H), 7.22 (dd, J=7.6, 1.0 Hz, 1H), 4.31 (dd, J=11.2, 2.4 Hz, 1H), 4.11-4.01 (m, 3H), 3.79 (s, 2H), 3.59-3.50 (m, 1H), 1.95-1.82 (m, 2H), 1.63-1.50 (m, 3H), 1.41-1.32 (m, 1H), 1.17 (t, J=7.2 Hz, 3H).

### Step 2: 2-(6-(tetrahydro-2H-pyran-2-yl)pyridin-2-yl)acetohydrazide

To a solution of ethyl 2-(6-(tetrahydro-2H-pyran-2-yl)pyridin-2-yl)acetate (150 mg, 0.6 mmol) in MeOH (10 mL) was added 98% hydrazine hydrate (39 mg, 1.2 mmol). The reaction mixture was heated at 80° C. for 2 h the the solvent was removed under reduced pressure. The residue was purified by prep-TLC (eluent: DCM:MeOH=15:1) to afford 2-(6-(tetrahydro-2H-pyran-2-yl)pyridin-2-yl)acetohydrazide (80 mg, 56%) as a yellow oil. LCMS m/z=236.1 [M+H]⁺; ¹H NMR (400 MHz, DMSO-d6) δ 9.21 (s, 1H), 7.70 (s, 1H), 7.23 (dd, J=19.2, 7.6 Hz, 2H), 4.30 (dd, J=11.2, 2.2 Hz, 1H), 4.23 (s, 2H), 4.03 (d, J=11.6 Hz, 1H), 3.57-3.50 (m, 3H), 2.05-1.80 (m, 3H), 1.59-1.51 (m, 2H), 1.44-1.34 (m, 1H).

### Synthesis of 2-(3,4-dichlorophenyl)-2,2-difluoroacetohydrazide

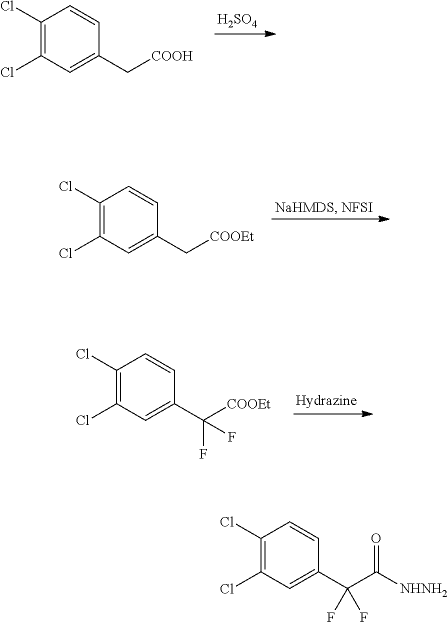

### Step 1: ethyl 2-(3, 4-dichlorophenyl) acetate

To a solution of ethyl 2-(3,4-dichlorophenyl)acetic acid (2.0 g, 9.75 mmol) in EtOH (20 mL) under N₂ was added a drop of conc. H₂SO₄. The reaction was heated at 80° C. for 5 hours then was cooled to room temperature and diluted with water (200 mL). The aqueous layer was extracted with EtOAc three times and the combined organic layers were washed with saturated sodium bicarbonate aqueous solution and brine, dried over Na₂SO₄, filtered and concentrated. The residue was purified by column chromatography on silica gel (eluent: Pet. Ether:EtOAc=50:1) to afford ethyl 2-(3,4-dichlorophenyl)acetate (2.0 g, 88%) as a colourless oil. ¹H NMR (400 MHz, CDCl₃) δ 7.43-7.37 (m, 2H), 7.12 (dd, J=8.2, 2.0 Hz, 1H), 4.17 (q, J=7.0 Hz, 2H), 3.56 (s, 2H), 1.26 (t, J=7.2 Hz, 3H).

### Step 2: ethyl 2-(3,4-dichlorophenyl)-2,2-difluoroacetate

To a solution of ethyl 2-(3,4-dichlorophenyl)acetate (500 mg, 2.15 mmol) in fresh distilled THF (5 mL) at −78° C. under a N₂ atmosphere was added NaHMDS (2.0 M, 2.36 mL). The reaction was stirred for 0.5 h, then a solution of NFSI (1.49 g, 4.72 mmol) in fresh distilled THF (10 mL) was added dropwise. The reaction was allowed to warm slowly to room temperature and stirred for another 2 h then was diluted with water (100 mL) and extracted with EtOAc. The combined organic layers were washed with saturated sodium bicarbonate aqueous solution and brine, dried over Na₂SO₄, filtered and concentrated. The residue was purified by column chromatography on silica gel (eluent: Pet. Ether:EtOAc=200:1) to afford ethyl 2-(3,4-dichlorophenyl)-2,2-difluoroacetate (100 mg, 17%) as a colourless oil. ¹H NMR (400 MHz, CDCl₃) δ 7.71 (d, J=2.2 Hz, 1H), 7.62-7.51 (m, 1H), 7.45 (dd, J=8.4, 2.2 Hz, 1H), 4.31 (q, J=7.2 Hz, 2H), 1.32 (td, J=7.0, 0.8 Hz, 3H). ¹⁹F NMR (376 MHz, CDCl₃) δ −103.99.

### Step 3: 2-(3,4-dichlorophenyl)-2,2-difluoroacetohydrazide

To a solution of ethyl 2-(3,4-dichlorophenyl)-2,2-difluoroacetate (50 mg, 0.186 mmol) in MeOH (2 mL) was added 98% hydrazine hydrate (0.3 mL). The mixture was stirred 30 min then was diluted with water (100 mL) and extracted with EtOAc. The combined organic layers were washed with brine, dried over Na₂SO₄, filtered and concentrated to afford 2-(3,4-dichlorophenyl)-2,2-difluoroacetohydrazide (30 mg, 64%) as a white solid. LCMS m/z=254.8 [M+H]⁺; ¹H NMR (400 MHz, DMSO-d₆) δ 10.30 (s, 1H), 7.85-7.76 (m, 2H), 7.56 (dd, J=8.4, 2.2 Hz, 1H), 4.58 (s, 2H); ¹⁹F NMR (376 MHz, DMSO-d₆) δ −102.28.

### Synthesis of 2-(6-(1-(trifluoromethyl)cyclopropyl)pyridin-2-yl)propanehydrazide

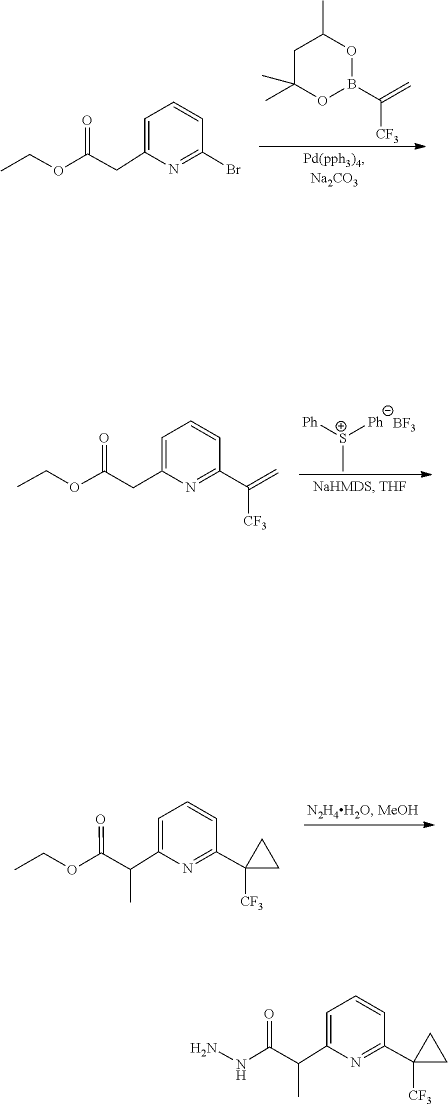

### Step 1: ethyl 2-(6-(3,3,3-trifluoroprop-1-en-2-yl)pyridin-2-yl)acetate

To a solution of ethyl 2-(6-bromopyridin-2-yl)acetate (1 g, 4.13 mmol) in a mixture of 1,4-dioxane (3 mL) and water (1.5 mL) was added 4,4,6-trimethyl-2-(3,3,3-trifluoroprop-1-en-2-yl)-1,3,2-dioxaborinane (1.65 g, 7.43 mmol), Na₂CO₃ (873 mg, 8.24 mmol) and Pd(PPh₃)₄ (238 mg, 0.21 mmol). The reaction was heated at 110° C. for 1.5 h in the microwave. The mixture was diluted with water (30 mL) and extracted with EtOAc (50 mL×2). The combined organic layers were dried over Na₂SO₄, filtered and concentrated. The residue was purified by column chromatography on silica gel (eluent: Pet. Ether/EtOAc=10/1, v/v) to afford ethyl 2-(6-(3,3,3-trifluoroprop-1-en-2-yl)pyridin-2-yl)acetate (960 mg, 97% yield) as a yellow oil. LCMS m/z=260.1 [M+H]⁺; ¹H NMR (400 MHz, Chloroform-d) δ 7.68 (t, J=7.8 Hz, 1H), 7.39 (d, J=8.0 Hz, 1H), 7.29 (d, J=7.8 Hz, 1H), 6.63 (m, 1H), 6.10 (m, 1H), 4.20 (q, J=7.2 Hz, 2H), 3.87 (s, 2H), 1.27 (t, J=7.2 Hz, 3H).

### Step 2: ethyl 2-(6-(1-(trifluoromethyl)cyclopropyl)pyridin-2-yl)propanoate

To a solution of ethyl 2-(6-(3,3,3-trifluoroprop-1-en-2-yl)pyridin-2-yl)acetate (960 mg, 3.7 mmol) and diphenyl(methyl)sulfonium tetrafluoroborate (1.39 g, 4.8 mmol) in anhydrous THF (25 mL) at 0° C. under a N₂ atmosphere was added NaHMDS (5.9 mL, 5.9 mmol). The reaction mixture was stirred for 10 min then allowed to warm to room temperature and stirred for another 1 h. The reaction was diluted with EtOAc (200 mL) and washed with brine (30 mL×2). The organic layer was dried over Na₂SO₄, filtered and concentrated. The residue was purified by prep-TLC (eluent: Pet. Ether:EtOAc=10:1) to afford ethyl 2-(6-(1-(trifluoromethyl)cyclopropyl)pyridin-2-yl)propanoate (160 mg, 15%) as a yellow oil. LCMS m/z=288.0 [M+H]⁺; ¹H NMR (400 MHz, Chloroform-d) δ 7.60 (t, J=7.8 Hz, 1H), 7.44 (d, J=8.0 Hz, 1H), 7.13 (d, J=7.8 Hz, 1H), 4.12 (q, J=6.8 Hz, 2H), 3.87 (q, J=7.2 Hz, 1H), 1.49 (d, J=7.2 Hz, 3H), 1.47-1.36 (m, 4H), 1.21 (t, J=7.0 Hz, 3H).

### Step 3: 2-(6-(1-(trifluoromethyl)cyclopropyl)pyridin-2-yl)propanehydrazide

A solution of ethyl 2-(6-(1-(trifluoromethyl)cyclopropyl)pyridin-2-yl)propanoate (110 mg, 0.38 mmol) and hydrazine hydrate (98%, 0.5 mL) in MeOH (2 mL) was stirred at room temperature for 6 h. The mixture was concentrated and purified by prep-TLC (eluent: DCM/MeOH=10:1) to afford 2-(6-(1-(trifluoromethyl)cyclopropyl)pyridin-2-yl)propanehydrazide (70 mg, 67%) as a white solid. LCMS m/z=274.0 [M+H]⁺; ¹H NMR (400 MHz, Chloroform-d) δ 7.65 (t, J=7.8 Hz, 1H), 7.41 (d, J=8.0 Hz, 1H), 7.19 (d, J=7.9 Hz, 1H), 3.78 (q, J=7.6 Hz, 1H), 1.56 (d, J=7.4 Hz, 3H), 1.49-1.44 (m, 2H), 1.40-1.34 (m, 2H).

### Synthesis of 4-(azidomethyl)-1,2-dichlorobenzene

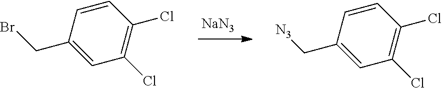 [SMILES: Clc1ccc(CBr)cc1Cl ; [N-]=[N+]=NCc1ccc(Cl)c(Cl)c1 ; "NaN3"]

### Step 1: 4-(azidomethyl)-1,2-dichlorobenzene

To a solution of 4-(bromomethyl)-1,2-dichlorobenzene (1.00 g, 4.17 mmol) in DMF (5 mL) was added NaN₃ (325 mg, 5.00 mmol) and the mixture was heated at 80° C. overnight. The mixture was diluted with water (50 mL) and extracted with EtOAc (50 mL×3). The combined organic layers were washed with brine, dried over Na₂SO₄, filtered and concentrated to afford 4-(azidomethyl)-1,2-dichlorobenzene (900 mg, contained DMF) as a yellow oil. ¹H NMR (400 MHz, DMSO-d₆) δ 7.70-7.64 (m, 2H), 7.41-7.35 (m, 1H), 4.50 (s, 2H).

### Synthesis of 2-chloropyridine 1-oxide

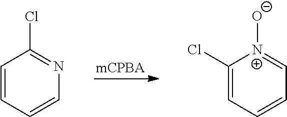 [SMILES: Clc1ccccn1 ; [O-][n+]1ccccc1Cl]

### Step 1: 2-chloropyridine 1-oxide

To a solution of 2-chloropyridine (500 mg, 4.4 mmol) in DCM (5 mL) was added 3-chlorobenzoperoxoic acid (1.36 g, 7.9 mmol). The reaction was stirred at room temperature for 12 h then was filtered and the filtrate was quenched with saturated sodium thiosulfate solution (30 mL) and extracted with DCM (50 mL×3). The combined organic layers were washed with brine, dried over Na₂SO₄, filtered and concentrated. The residue obtained was purified by column (eluent: DCM/MeOH=20/1, v/v) to afford 2-chloropyridine 1-oxide (450 mg, 80%) as a brown oil. LCMS m/z=130.3 [M+H]⁺; ¹H NMR (400 MHz, DMSO-d₆) δ 8.44 (dd, J=6.4, 1.6 Hz, 1H), 7.78 (dd, J=8.0, 2.1 Hz, 1H), 7.44-7.31 (m, 2H).

### Synthesis of (S)-2-(3,5-dimethyl-1H-pyrazol-1-yl)propanoic acid

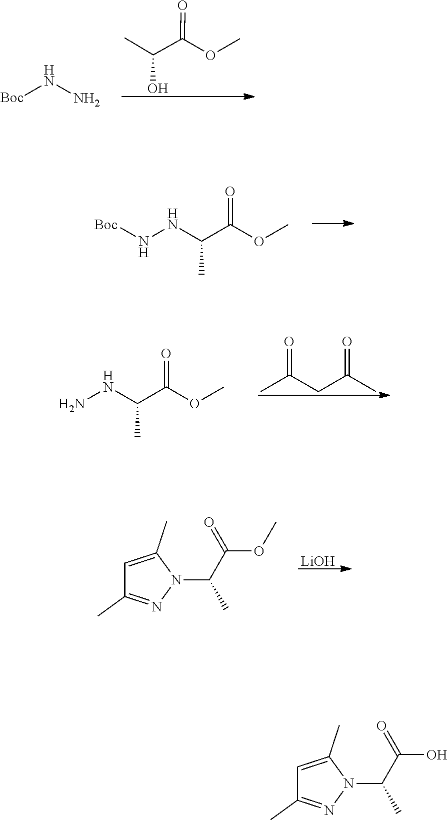

### Step 1: tert-butyl (S)-2-(1-methoxy-1-oxopropan-2-yl)hydrazine-1-carboxylate

A solution of methyl (R)-2-hydroxypropanoate (1.0 g, 9.61 mmol) and 2,6-dimethylpyridine (2.6 mL, 22.09 mmol) in DCM (10.0 mL) at 0° C. under an atmosphere of nitrogen was treated with trifluoromethanesulfonic anhydride (1.8 mL, 10.57 mmol). After 20 min, a solution of tert-Butyl carbazate (1.27 g, 9.61 mmol) in DCM (5.0 mL) was added dropwise over 20 min and the mixture stirred for 6 hours at 0° C. The solvent was removed under reduce pressure and the residue obtained diluted with water (30 mL) and extracted with ether (50 mL×3). The combined organic layers was washed with brine, dried over Na₂SO₄, filtered and concentrated. The residue was purified by column chromatography on silica gel (eluent: Pet. Ether/EtOAc=10/1 to 5/1) to afford tert-butyl (S)-2-(1-methoxy-1-oxopropan-2-yl)hydrazine-1-carboxylate (450 mg, 21%) as a yellow oil. ¹H NMR (400 MHz, DMSO-d₆) δ 8.20 (s, 1H), 4.69 (s, 1H), 3.61 (s, 3H), 3.55 (q, J=7.1 Hz, 1H), 1.38 (s, 9H), 1.13 (d, J=6.9 Hz, 3H).

### Step 2: methyl amino-L-alaninate

To a solution of tert-butyl (S)-2-(1-methoxy-1-oxopropan-2-yl)hydrazine-1-carboxylate (100 mg, 0.46 mmol) in DCM (2.0 mL) was added TFA (2.0 mL). The reaction was stirred at room temperature for 2 hours then the solvent was removed under reduced pressure to afford methyl amino-L-alaninate (80 mg, quant.) which was used directly in the next step.

### Step 3: methyl (S)-2-(3,5-dimethyl-1H-pyrazol-1-yl)propanoate

A mixture of methyl amino-L-alaninate (50 mg, 0.42 mmol) in pentane-2,4-dione (0.3 mL) was heated at 120° C. for 20 min in the microwave. The solvent was removed under reduced pressure to afford methyl (S)-2-(3,5-dimethyl-1H-pyrazol-1-yl)propanoate (40 mg, 52%) which was used directly in the next step. LCMS m/z=182.9 [M+H]⁺.

### Step 4: (S)-2-(3,5-dimethyl-1H-pyrazol-1-yl)propanoic acid

To a solution of methyl (S)-2-(3,5-dimethyl-1H-pyrazol-1-yl)propanoate (40 mg, 0.22 mmol) in a mixture of THF and water (2 mL/1 mL) was added a solution of lithium hydroxide monohydrate (10 mg, 0.24 mmol). The mixture was stirred at room temperature for 2 h then diluted with water (10 mL) and extracted with ether (15 mL×2). The aqueous layer was collected and acidified to pH˜1 with 1M HCl then extracted with EtOAc (25 mL×3). The combined organic layers were washed with brine, dried over Na₂SO₄, filtered and concentrated to afford crude (S)-2-(3,5-dimethyl-1H-pyrazol-1-yl)propanoic acid (20 mg, 54%) as a yellow oil which was used directly in the next step. LCMS m/z=169.1 [M+H]⁺; ¹H NMR (400 MHz, CD₃OD) δ 6.00 (s, 1H), 5.14 (q, J=7.2 Hz, 1H), 2.29 (s, 3H), 2.24 (s, 3H), 1.74 (d, J=7.2 Hz, 3H).

### Synthesis of (cis)-3-ethoxycyclobutane-1-carboxylic acid

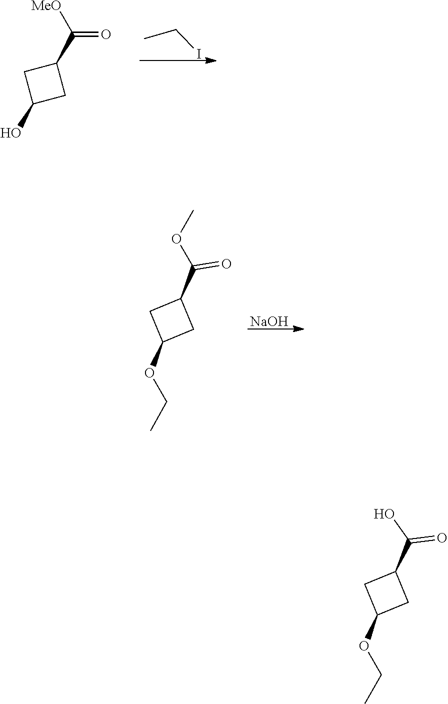

### Step 1: methyl (cis)-3-ethoxycyclobutane-1-carboxylate

To a solution of methyl (cis)-3-hydroxycyclobutane-1-carboxylate (100 mg, 1.54 mmol) in DCM (4 mL) at 0° C. was added DIPEA (338 mg, 5.24 mmol), iodoethane (382 mg, 4.93 mmol), and silver trifluoromethanesulfonate (560 mg, 4.62 mmol). The resulting mixture was stirred for 2 h at 0° C. The reaction was quenched with water (10 mL) and extracted with DCM (20 mL×3). The combined organic layers were washed with water and brine, dried over Na₂SO₄, filtered and concentrated to afford methyl (cis)-3-ethoxycyclobutane-1-carboxylate (68 mg, 56%) as a brown oil which was used directly in the next step.

### Step 2: (cis)-3-ethoxycyclobutane-1-carboxylic acid

To a solution of methyl (cis)-3-ethoxycyclobutane-1-carboxylate (68 mg, 0.38 mmol) in MeOH (2 mL) was added aqueous NaOH (10%, 1 mL). The reaction was stirred overnight then the solvent was removed under reduced pressure. The residue obtained was diluted with water and the pH adjusted to ˜1 by addition of 1M HCl. The aqueous layer was extracted with EtOAc (20 mL×3) and the combined organic layers washed with water, brine, dried over Na₂SO₄ and concentrated to afford (cis)-3-ethoxycyclobutane-1-carboxylic acid (38 mg, 65%) as a yellow oil. ¹H NMR (400 MHz, CDCl₃) δ 3.88 (tt, J=8.0, 6.7 Hz, 1H), 3.40 (q, J=7.0 Hz, 2H), 2.70-2.61 (m, 1H), 2.53-2.49 (m, 2H), 2.30-2.18 (m, 2H), 1.18 (t, J=7.0 Hz, 3H).

### Synthesis of 2-(6-(tetrahydro-2H-pyran-4-yl)pyridin-2-yl)acetohydrazide

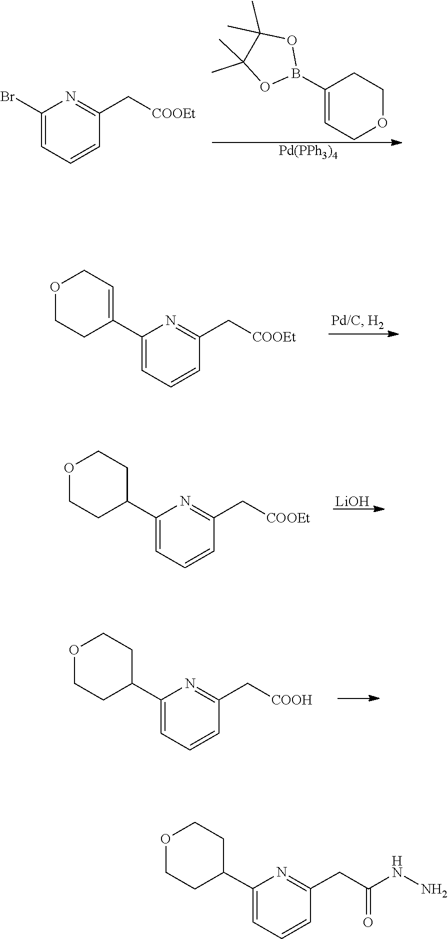

### Step 1: ethyl 2-(6-(3,6-dihydro-2H-pyran-4-yl)pyridin-2-yl)acetate

To a solution of ethyl 2-(6-bromopyridin-2-yl)acetate (500 mg, 2.05 mmol), Na₂CO₃ (434.0 mg, 4.10 mmol) and Pd(PPh₃)₄ (473.0 mg, 0.41 mmol) in a mixture of DME (10 mL) and H₂O (2 mL) was added 2-(3,6-dihydro-2H-pyran-4-yl)-4,4,5,5-tetramethyl-1,3,2-dioxaborolane (468 mg, 2.25 mmol). The resulting mixture was heated at 90° C. for 7 hours under N₂ then was cooled to room temperature, diluted with water (200 mL) and extracted with EtOAc three times. The combined organic layers were washed with water and brine, dried over Na₂SO₄, filtered and concentrated. The mixture was purified by column chromatography on silica gel (eluent: Pet. Ether:EtOAc=3:1) to afford ethyl 2-(6-(3,6-dihydro-2H-pyran-4-yl)pyridin-2-yl)acetate (340 mg, 68%) as a yellow oil. ¹H NMR (400 MHz, CDCl₃) δ 7.58 (t, J=7.8 Hz, 1H), 7.22 (d, J=8.0 Hz, 1H), 7.11 (d, J=7.6 Hz, 1H), 6.73 (tt, J=3.8, 1.8 Hz, 1H), 4.18 (q, J=7.2 Hz, 2H), 3.83 (s, 2H), 2.48 (tq, J=6.4, 2.4 Hz, 2H), 2.25 (dtd, J=9.0, 6.2, 2.6 Hz, 2H), 2.16-2.03 (m, 2H), 1.27 (t, J=7.2 Hz, 3H).

### Step 2: ethyl 2-(6-(tetrahydro-2H-pyran-4-yl)pyridin-2-yl)acetate

To a solution of ethyl 2-(6-(3,6-dihydro-2H-pyran-4-yl)pyridin-2-yl)acetate (50 mg, 0.202 mmol) in MeOH (3 mL) was added 10% Pd/C (15 mg). The reaction was stirred at room temperature under a H₂ atmosphere overnight. The catalyst was removed by filtration through celite and the filtrate concentrated to afford ethyl 2-(6-(tetrahydro-2H-pyran-4-yl)pyridin-2-yl)acetate (47 mg, 94%) as a colorless oil, which was used in the next step directly. LCMS m/z=250.2 [M+H]⁺; ¹H NMR (400 MHz, CDCl₃) δ 7.62 (s, 1H), 7.22-7.02 (m, 2H), 4.19 (q, J=7.0 Hz, 2H), 4.08 (dt, J=11.4, 3.4 Hz, 2H), 3.84 (s, 2H), 3.64-3.45 (m, 2H), 2.96 (s, 1H), 1.87 (h, J=4.4, 3.8 Hz, 4H), 1.26 (d, J=7.0 Hz, 3H).

### Step 3: 2-(6-(tetrahydro-2H-pyran-4-yl)pyridin-2-yl)acetic acid

To a solution of ethyl 2-(6-(tetrahydro-2H-pyran-4-yl)pyridin-2-yl)acetate (60 mg, 0.241 mmol) in a mixture of THF (2 mL) and water (0.5 mL) was added LiOH·H₂O (12 mg, 0.289 mmol). The reaction was stirred at room temperature for 1 h then diluted with water (3 mL) and extracted with ether (30 mL). The aqueous layer was collected and acidified to pH˜2 with 1M HCl then extracted with EtOAc (25 mL×2). The combined organic layers were washed with brine, dried over Na₂SO₄, filtered and concentrated to afford 2-(6-(tetrahydro-2H-pyran-4-yl)pyridin-2-yl)acetic acid (50 mg, 94%) as a white solid. LCMS m/z=222.1 [M+H]⁺.

### Step 4: 2-(6-(tetrahydro-2H-pyran-4-yl)pyridin-2-yl)acetohydrazide

To a solution of 2-(6-(tetrahydro-2H-pyran-4-yl)pyridin-2-yl)acetic acid (50 mg, 0.226 mmol) in DMF (3 mL) was added CDI (55.0 mg, 0.339 mmol). The reaction was stirred 30 min then 98% hydrazine hydrate (0.3 mL) was added. The reaction was stirred for a further 5 hours then was diluted with water (5 mL) and extracted with EtOAc. The combined organic layers were washed with brine, dried over Na₂SO₄, filtered and concentrated. The residue obtained was purified by prep-TLC (DCM/MeOH=10/1) to afford 2-(6-(tetrahydro-2H-pyran-4-yl)pyridin-2-yl)acetohydrazide (120 mg, 80%) as a white solid. LCMS m/z=236.2 [M+H]⁻; ¹H NMR (400 MHz, DMSO-d₆) δ 9.21 (s, 1H), 7.64 (d, J=2.2 Hz, 1H), 7.15 (dd, J=13.4, 7.8 Hz, 2H), 4.26-4.09 (m, 2H), 3.94 (dt, J=11.2, 3.4 Hz, 2H), 3.52 (s, 2H), 3.43 (ddd, J=11.4, 8.2, 5.2 Hz, 2H), 2.96-2.81 (m, 1H), 1.80-1.69 (m, 4H).

### Synthesis of 2-(4-cyclopentyl-1H-pyrazol-1-yl) propanoic acid

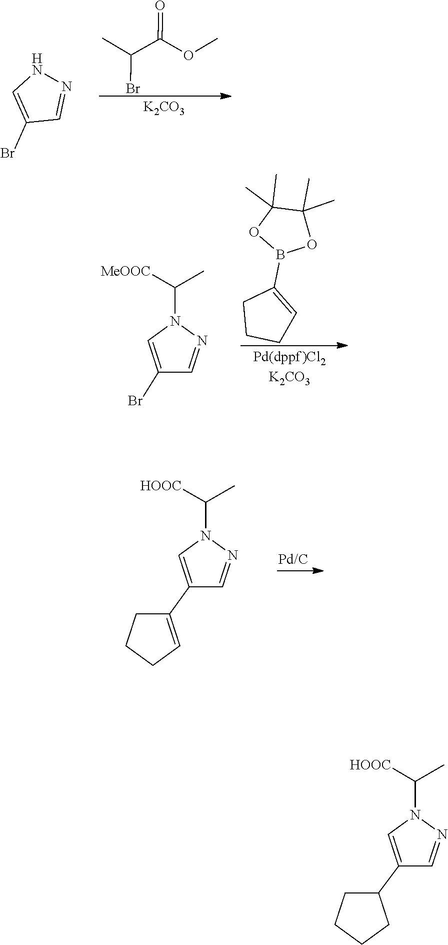

### Step 1: methyl 2-(4-bromo-1H-pyrazol-1-yl) propanoate

To a solution of 4-bromo-1H-pyrazole (5.0 g, 34.0 mmol) and K₂CO₃ (14.1 g, 102.0 mmol) in DMF (50 mL) under a N₂ atmosphere was added methyl 2-bromopropanoate (6.82 g, 40.82 mmol). The reaction was stirred for 5 hours then was diluted with water (100 mL) and extracted with EtOAc (100 mL×3), The combined organic layers were washed with water and brine, dried over Na₂SO₄, filtered and concentrated. The residue obtained was purified by column chromatography on silica gel (eluent: Pet. Ether:EtOAc=3:1) to afford methyl 2-(4-bromo-1H-pyrazol-1-yl)propanoate (5.1 g, 64%) as a colourless oil. LCMS m/z=233.1 [M+H]⁺; ¹H NMR (400 MHz, CDCl₃) δ 7.33 (s, 1H), 7.26 (s, 1H), 4.84 (qd, J=7.2, 1.0 Hz, 1H), 3.52 (d, J=1.0 Hz, 3H), 1.54 (dd, J=7.4, 1.0 Hz, 3H).

### Step 2: 2-(4-(cyclopent-1-en-1-yl)-1H-pyrazol-1-yl)propanoic acid

To a solution of methyl 2-(4-bromo-1H-pyrazol-1-yl)propanoate (1.0 g, 4.19 mmol), K₂CO₃ (1.78 g, 12.87 mmol) and Pd(dppf)Cl₂ (0.72 g, 0.43 mmol) in a mixture of 1,4-dioxane (10 mL) and water (2 mL) under N₂ atmosphere was added 2-(cyclopent-1-en-1-yl)-4,4,5,5-tetramethyl-1,3,2-dioxaborolane (1.25 g, 6.44 mmol). The reaction mixture was heated at 100° C. overnight then was diluted with water (50 mL) and extracted with Et₂O (50 mL×3). The aqueous layer was collected and acidified to pH˜2 with 1M HCl then extracted with EtOAc (100 mL×2). The combined organic layers were washed with brine, dried over Na₂SO₄, filtered and concentrated. The residue obtained was purified by RP column to afford 2-(4-(cyclopent-1-en-1-yl)-1H-pyrazol-1-yl)propanoic acid (500 mg, 57%) as a yellow oil. LCMS m/z=207.2 [M+H]⁺.

### Step 3: 2-(4-cyclopentyl-1H-pyrazol-1-yl) propanoic acid

To a solution of 2-(4-(cyclopent-1-en-1-yl)-1H-pyrazol-1-yl) propanoic acid (200 mg, 0.97 mmol) in MeOH (2 mL) was added 10% Pd/C (30 mg). The reaction was stirred under a H₂ atmosphere for 5 h then the catalyst was removed by filtration through celite. The filtrate was concentrated to afford 2-(4-cyclopentyl-1H-pyrazol-1-yl) propanoic acid (190 mg, 94%) which was used in the next step without further purification. LCMS m/z=209.2 [M+H]⁺; ¹H NMR (400 MHz, DMSO-d₆) δ 7.54 (s, 1H), 7.28 (s, 1H), 4.99 (q, J=7.2 Hz, 1H), 2.85 (p, J=8.4 Hz, 1H), 2.03-1.87 (m, 2H), 1.72-1.54 (m, 7H), 1.50-1.35 (m, 2H).

### Synthesis of 2-(1-(tetrahydro-2H-pyran-2-yl)-1H-pyrazol-5-yl) acetohydrazide

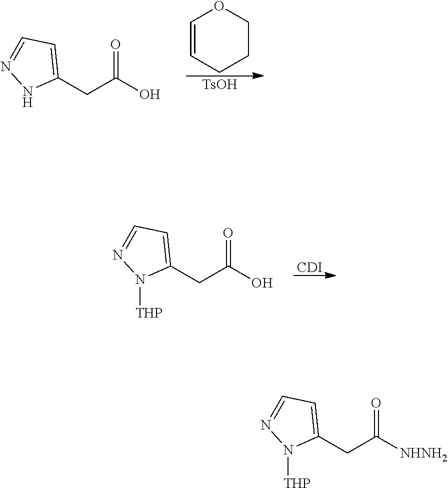

### Step 1: 2-(1-(tetrahydro-2H-pyran-2-yl)-1H-pyrazol-5-yl) acetic acid

To a solution of 2-(1H-pyrazol-5-yl)acetic acid (250 mg, 1.98 mmol) and 3,4-dihydro-2H-pyran (333.5 mg, 3.96 mmol) in a mixture of DMF (1 mL) and EtOAc (5 mL) was added p-TsOH (34.5 mg, 0.2 mmol). The resulting mixture was stirred for 10 hours then was diluted with water (200 mL) and extracted with EtOAc three times. The combined organic layers were washed with saturated sodium bicarbonate aqueous solution and brine, dried over Na₂SO₄, filtered and concentrated afford 2-(1-(tetrahydro-2H-pyran-2-yl)-1H-pyrazol-5-yl)acetic acid (180 mg, crude) as a yellow oil. LCMS m/z=211.2[M+H]⁺; ¹H NMR (400 MHz, DMSO-d₆) δ 12.31 (s, 1H), 7.76 (d, J=2.4 Hz, 1H), 6.18 (d, J=2.4 Hz, 1H), 5.31 (dd, J=10.2, 2.4 Hz, 1H), 3.96-3.85 (m, 1H), 3.64-3.55 (m, 1H), 3.51 (s, 2H), 2.05 (tdd, J=12.6, 10.0, 3.8 Hz, 1H), 1.96-1.80 (m, 2H), 1.64 (dddt, J=15.2, 11.6, 8.2, 3.4 Hz, 1H), 1.51 (td, J=7.2, 6.4, 4.4 Hz, 2H).

### Step 2: 2-(1-(tetrahydro-2H-pyran-2-yl)-1H-pyrazol-5-yl) acetohydrazide

To a solution of 2-(1-(tetrahydro-2H-pyran-2-yl)-1H-pyrazol-5-yl)acetic acid (180 mg, 0.86 mmol) in THE (5 mL) was added CDI (208.2 mg, 1.28 mmol). The mixture was stirred for 30 mins then 98% hydrazine hydrate (0.5 mL) was added and stirring continued for 6 hours. The reaction was diluted with water (100 mL) and extracted with DCM three times. The combined organic layers were washed with saturated sodium bicarbonate aqueous solution and brine, dried over Na₂SO₄, filtered and concentrated. The mixture was purified by prep-TLC (eluent: DCM:MeOH=10:1) to afford 2-(1-(tetrahydro-2H-pyran-2-yl)-1H-pyrazol-5-yl)acetohydrazide (162.0 mg, 84%) as a white solid. LCMS m/z=225.1[M+H]⁺.

### Synthesis of 2-(2,3-dihydrobenzo[b][1,4]dioxin-5-yl)acetohydrazide

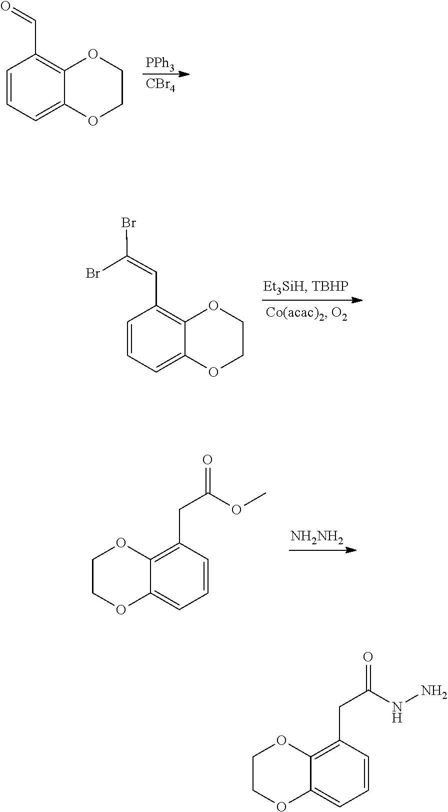

### Step 1: 5-(2,2-dibromovinyl)-2,3-dihydrobenzo[b][1,4]dioxine

To a solution of 2,3-dihydrobenzo[b][1,4]dioxine-5-carbaldehyde (300.0 mg, 1.83 mmol) in DCM (5 mL) at 0° C. under N₂ was added CBr₄ (1.21 g, 73.65 mmol) (2.04 g, 6.17 mmol). PPh₃ (1.92 g, 7.31 mmol) was added and the reaction stirred at 0° C. for 6 hours. The mixture was diluted with water (100 mL) and extracted with DCM three times. The combined organic layers were washed with water, brine, dried over Na₂SO₄, filtered and concentrated. The residue was purified by column chromatography on silica gel (eluent: Pet. Ether:EtOAc=50:1) to afford 5-(2,2-dibromovinyl)-2,3-dihydrobenzo[b][1,4]dioxine (500 mg, 86%) as a colourless oil. ¹H NMR (400 MHz, CDCl₃) δ 7.55 (s, 1H), 7.30-7.26 (m, 1H), 6.89-6.80 (m, 2H), 4.39-4.20 (m, 4H).

### Step 2: methyl 2-(2,3-dihydrobenzo[b][1,4]dioxin-5-yl)acetate

To a solution of 5-(2,2-dibromovinyl)-2,3-dihydrobenzo[b][1,4]dioxine (300 mg, 0.937 mmol) and Et₃SiH (545.09 mg, 4.69 mmol) in MeOH (3.13 mL) was added Co(acac)2 (334.0 mg, 0.937 mmol) and TBHP in decane (5.0-6.0 mol, 0.5 mL). The resulting mixture was stirred for 14 hours under and oxygen atmosphere. The reaction was diluted with water (200 mL) and extracted with EtOAc three times. The organic layers were washed with water and brine, dried over Na₂SO₄, filtered and concentrated. The residue was purified by prep-TLC (eluent: Pet. Ether:EtOAc=20:1, v/v) to afford methyl 2-(2,3-dihydrobenzo[b][1,4]dioxin-5-yl)acetate (110 mg, 56%) as a colourless oil. LCMS m/z=209.1 [M+H]⁺; ¹H NMR (400 MHz, DMSO-d₆) δ 6.79-6.73 (m, 3H), 4.26-4.18 (m, 4H), 3.60 (s, 3H), 3.58 (s, 2H).

### Step 3: 2-(2,3-dihydrobenzo[b][1,4]dioxin-5-yl)acetohydrazide

To a solution of methyl 2-(2,3-dihydrobenzo[b][1,4]dioxin-5-yl)acetate (100 mg, 0.480 mmol) in MeOH (5 mL) was added 98% hydrazine hydrate (0.5 mL). The mixture was stirred for 30 min then was partitioned against water and extracted with DCM:MeOH=5:1. The combined organic layers were washed with water and brine, dried over Na₂SO₄, filtered and concentrated to afford 2-(2,3-dihydrobenzo[b][1,4]dioxin-5-yl)acetohydrazide (80 mg, 80%) as white solid. LCMS m/z=209.2 [M+H]⁺; ¹H NMR (400 MHz, DMSO-d₆) δ 9.01 (s, 1H), 6.74-6.70 (m, 3H), 4.26-4.16 (m, 6H), 3.30-3.32 (m, 2H).

### Synthesis of 2-(1H-indol-2-yl)acetohydrazide

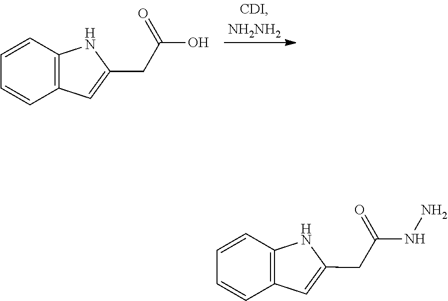 [SMILES: O=C(O)Cc1cc2ccccc2[nH]1 ; NNC(=O)Cc1cc2ccccc2[nH]1]

### Step 1: 2-(1H-indol-2-yl)acetohydrazide

To a solution of 2-(1H-indol-2-yl)acetic acid (1.0 g, 5.7 mmol) in THE (15 mL) at 0° C. was added CDI (1.1 g, 6.8 mmol). The mixture was stirred 1 h then hydrazine hydrate (98%, 0.86 g, 17.1 mmol) was added dropwise. The reaction was stirred a further 14 h then was diluted with water (30 mL) and extracted with EtOAc (30 mL×2). The combined organic layers were washed with water and brine, dried over Na₂SO₄, filtered and concentrated. The residue was triturated with ether (50 mL) to afford 2-(1H-indol-2-yl)acetohydrazide (0.36 g, 33%) as a white solid. LCMS m/z=190.2[M+H]⁺.

### Synthesis of 2-(2,3-dihydrobenzo[b][1,4]dioxin-6-yl)acetohydrazide

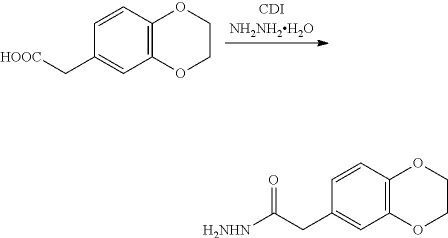

### Step 1: 2-(2,3-dihydrobenzo[b][1,4]dioxin-6-yl)acetohydrazide

To a solution of 2-(2,3-dihydrobenzo[b][1,4]dioxin-6-yl)acetic acid (100 mg, 0.51 mmol) in THF (2 mL) was added CDI (125 mg, 0.77 mmol) and the mixture stirred at room temperature for 30 min. 98% hydrazine hydrate (0.3 mL) was added and the reaction was stirred for another 4 h. The mixture was diluted with water (10 mL) and extracted with DCM/MeOH=5/1 (30 mL×3). The combined organic layers were washed with brine, dried over Na₂SO₄, filtered and concentrated to afford 2-(2,3-dihydrobenzo[b][1,4]dioxin-6-yl)acetohydrazide (92 mg, 86%) as a colorless semisolid. LCMS m/z=208.8 [M+H]⁺.

### Synthesis of 2-(chroman-6-yl)acetohydrazide

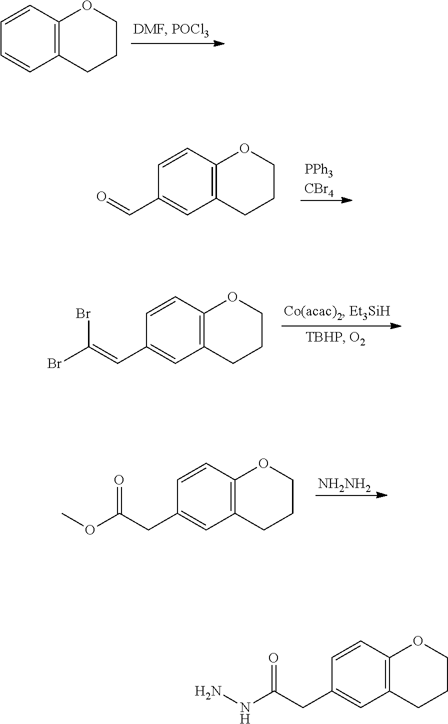

### Step 1: Chromane-6-carbaldehyde

To a solution of chromane (1.0 g, 7.45 mmol) and DMF (1.08 g, 14.9 mmol) in DCE (20 mL) was added POCl₃ (2.28 g, 14.9 mmol) dropwise over 30 mins keeping the rection temperature below 50° C. The resulting mixture was heated at 85° C. for 10 hours then was cooled to room temperature, diluted with water (50 mL) and extracted with EtOAc. The combined organic layers were washed with saturated sodium bicarbonate aqueous solution and brine, dried over Na₂SO₄, filtered and concentrated. The residue was purified by column chromatography on silica gel (eluent: Pet. Ether:EtOAc=10:1) to afford Chromane-6-carbaldehyde (560 mg, 47%) as a colorless oil. LCMS m/z=163.2 [M+H]⁺; ¹H NMR (400 MHz, CDCl₃) δ 9.83 (s, 1H), 7.62 (dd, J=11.2, 3.0 Hz, 2H), 6.89 (d, J=8.2 Hz, 1H), 4.32-4.23 (m, 2H), 2.85 (t, J=6.4 Hz, 2H), 2.08-2.00 (m, 2H)

### Step 2: 6-(2,2-dibromovinyl)chromane

To a solution of Chromane-6-carbaldehyde (500 mg, 3.08 mmol) in DCM (5 mL) at 0° C. was added CBr₄ (2.04 g, 6.17 mmol). PPh₃ (3.23 g, 12.33 mmol) was added and the reaction stirred at 0° C. for 6 hours. The reaction was diluted with water (100 mL) and extracted with DCM three times. The combined organic layers were washed with saturated sodium bicarbonate and brine, dried over Na₂SO₄, filtered and concentrated. The residue was purified by column chromatography on silica gel (eluent: Pet. Ether:EtOAc=50:1) to afford 6-(2,2-dibromovinyl)chromane (850 mg, 87%) as a colorless oil. ¹H NMR (400 MHz, DMSO-d₆) δ 7.61 (s, 1H), 7.40-7.31 (m, 2H), 6.76 (d, J=8.4 Hz, 1H), 4.23-4.05 (m, 2H), 2.74 (t, J=6.4 Hz, 2H), 2.02-1.86 (m, 2H).

### Step 3: methyl 2-(chroman-6-yl) acetate

To a solution of 6-(2,2-dibromovinyl)chromane (800 mg, 2.52 mmol) in MeOH (8.4 mL) was added Co(acac)₂ (897 mg, 2.52 mmol), Et₃SiH (1.46 g, 12.58 mmol) and TBHP in decane (5.0-6.0 mol, 0.5 mL). The resulting mixture was stirred for 14 hours under an oxygen atmosphere. The reaction was diluted with water (200 mL) and extracted with EtOAc three times. The combined organic layers were washed with saturated sodium bicarbonate aqueous solution and brine, dried over Na₂SO₄, filtered and concentrated. The residue was purified by column chromatography on silica gel (eluent: Pet. Ether:EtOAc=20:1) to afford methyl 2-(chroman-6-yl)acetate (250 mg, 48%) as a colourless oil. LCMS m/z=207.2[M+H]⁺; ¹H NMR (400 MHz, DMSO-d₆) δ 6.94-6.88 (m, 2H), 6.71-6.58 (m, 1H), 4.14-4.03 (m, 2H), 3.59 (s, 3H), 3.52 (s, 2H), 2.70 (t, J=6.4 Hz, 2H), 1.98-1.74 (m, 2H).

### Step 4: 2-(chroman-6-yl)acetohydrazide

To a solution of methyl 2-(chroman-6-yl)acetate (150 mg, 0.727 mmol) in MeOH (3 mL) was added 98% hydrazine hydrate (0.3 mL). The mixture was stirred for 30 min then was diluted with water and the aqueous extracted with EtOAc. The combined organic layers were washed with brine, dried over Na₂SO₄, filtered and concentrated to afford ethyl 2-(3-fluoro-6-(tetrahydro-2H-pyran-4-yl)pyridin-2-yl)acetate (120 mg, 80%) as a white solid. LCMS m/z=207.2 [M+H]⁺; ¹H NMR (400 MHz, DMSO-d₆) δ 9.09 (s, 1H), 6.91 (dd, J=4.4, 2.4 Hz, 2H), 6.70-6.54 (m, 1H), 4.16 (d, J=4.4 Hz, 2H), 4.11-4.05 (m, 2H), 3.20 (s, 2H), 2.69 (t, J=6.4 Hz, 2H), 1.88 (dt, J=11.4, 6.2 Hz, 2H).

### Synthesis of 3-(3,4-dichlorophenyl)propanehydrazide

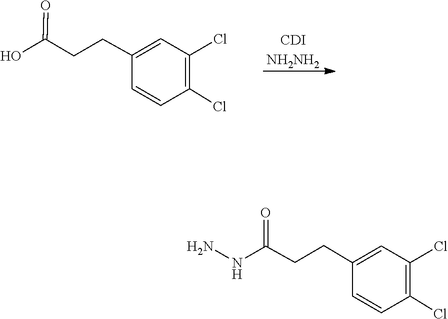

### Step 1: 3-(3,4-dichlorophenyl)propanehydrazide

To a solution of 3-(3,4-dichlorophenyl)propanoic acid (100 mg, 0.46 mmol) in THF (2 mL) was added CDI (111 mg, 0.69 mmol). The mixture was stirred at room temperature for 30 min then hydrazine hydrate 98% (0.5 mL) was added and the reaction stirred for another 4 h. The mixture was diluted with water (10 mL) and extracted with DCM/MeOH=5/1 (30 mL*3). The combined organic layers were washed with brine, dried over Na₂SO₄, filtered and concentrated to afford 3-(3,4-dichlorophenyl)propanehydrazide (134 mg, quant.) as a yellow oil. LCMS m/z=232.8 [M+H]⁺.

### Synthesis of 2-(4-chlorophenyl)-2,2-difluoroacetohydrazide

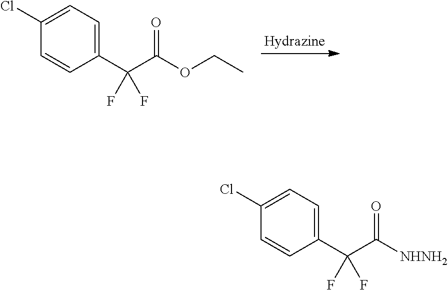

### Step 1: 2-(4-chlorophenyl)-2,2-difluoroacetohydrazide

To a solution of ethyl 2-(4-chlorophenyl)-2,2-difluoroacetate (500 mg, 2.13 mmol) in MeOH (3 mL) was added 98% hydrazine hydrate (0.3 mL). The mixture was stirred at room temperature for 30 min then was diluted with water (20 mL) and extracted with EtOAc (50 mL×3). The combined organic layers were washed with brine, dried over Na₂SO₄, filtered and concentrated to afford 2-(4-chlorophenyl)-2,2-difluoroacetohydrazide (460 mg, 98%) as a white solid. LCMS m/z=221.0 [M+H]⁺; ¹HNMR (400 MHz, DMSO-d₆) δ 10.29 (s, 1H), 7.67-7.52 (m, 4H), 4.55 (s, 2H). ¹⁹F NMR (376 MHz, DMSO-d6) δ −101.87.

### Synthesis of 2,2-difluoro-2-phenylacetohydrazide

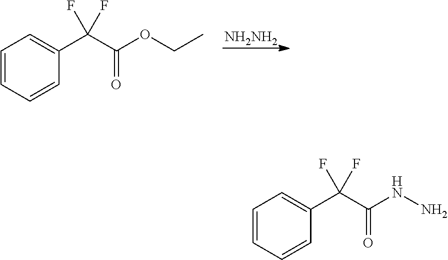

### Step 1: 2,2-difluoro-2-phenylacetohydrazide

To a solution of ethyl 2,2-difluoro-2-phenylacetate (200 mg, 1.0 mmol) in MeOH (4 mL) was added 98% hydrazine hydrate (50 mg, 3.0 mmol). The reaction was stirred at room temperature for 30 min then diluted with water (10 mL) and extracted with DCM/MeOH=5/1 (30 mL×3). The combined organic layers were washed with brine, dried over Na₂SO₄, filtered and concentrated to afford 2-(2,3-dihydrobenzo[b][1,4]dioxin-6-yl) acetohydrazide (134 mg, 72%) as a yellow oil. LCMS m/z=187.1 [M+H]⁺.

### Synthesis of 2,2-difluoro-2-(2-methoxyphenyl)acetohydrazide

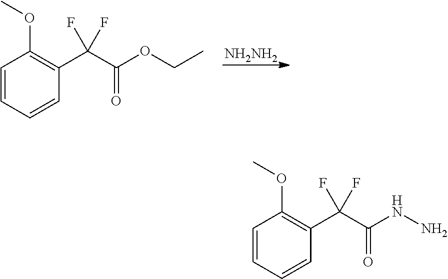

### Step 1: 2,2-difluoro-2-(2-methoxyphenyl)acetohydrazide

To a solution of ethyl 2,2-difluoro-2-(2-methoxyphenyl)acetate (100 mg, 0.43 mmol) in MeOH (1 mL) was added hydrazine hydrate 98% (43 mg, 0.86 mmol). The reaction was stirred for 30 min then diluted with water (10 mL) and extracted with DCM/MeOH=5/1 (30 mL×3). The combined organic layers were washed with brine, dried over Na₂SO₄, filtered and concentrated under reduce pressure to afford 2,2-difluoro-2-(2-methoxyphenyl)acetohydrazide (94 mg, quant.) as a yellow oil. LCMS m/z=217.2 [M+H]⁺.

### Synthesis of 2-(3,4-difluorophenyl)-2,2-difluoroacetohydrazide

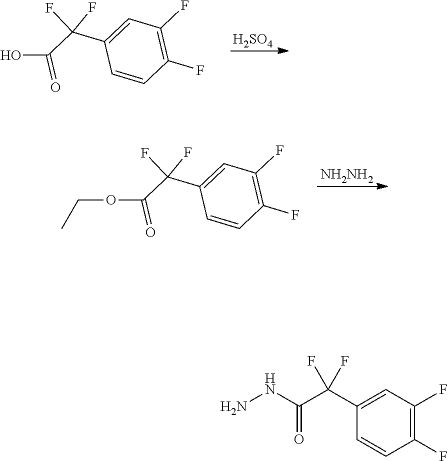

### Step 1: ethyl 2-(3,4-difluorophenyl)-2,2-difluoroacetate

To a solution of 2-(3,4-difluorophenyl)-2,2-difluoroacetic acid (100 mg, 0.480 mmol) in EtOH (5 mL) was added a drop H₂SO₄. The reaction was heated at 80° C. for 5 hours then was cooled to room temperature, diluted with water (100 mL) and extracted with EtOAc three times. The combined organic layers were washed with saturated Na₂CO₃ solution, brine, dried over Na₂SO₄, filtered and concentrated. The mixture was purified by column chromatography on silica gel (eluent: Pet. Ether:EtOAc=20:1) to afford ethyl 2-(3,4-difluorophenyl)-2,2-difluoroacetate (105 mg, 93%) as a colourless oil. ¹H NMR (400 MHz, DMSO-d₆) δ 7.74 (td, J=7.8, 3.8 Hz, 1H), 7.64 (q, J=8.8 Hz, 1H), 7.50 (s, 1H), 4.31 (q, J=7.2 Hz, 2H), 1.22 (t, J=7.2 Hz, 3H).

### Step 2: 2-(3,4-difluorophenyl)-2,2-difluoroacetohydrazide

To a solution of ethyl 2-(3,4-difluorophenyl)-2,2-difluoroacetate (105 mg, 0.44 mmol) in MeOH (3 mL) was added 98% hydrazine hydrate (0.3 mL). The reaction was stirred for 30 min then water was added and the aqueous extracted with EtOAc. The combined organic layers were washed with brine, dried over Na₂SO₄, filtered and concentrated to afford 2-(3,4-difluorophenyl)-2,2-difluoroacetohydrazide (95 mg, 98%) as a white solid. LCMS m/z=223.1 [M+H]⁺; ¹H NMR (400 MHz, DMSO-d₆) δ 10.28 (s, 1H), 7.75-7.57 (m, 2H), 7.50-7.40 (m, 1H), 4.60 (s, 2H).

### Synthesis of 2,2-difluoro-2-(naphthalen-1-yl)acetohydrazide

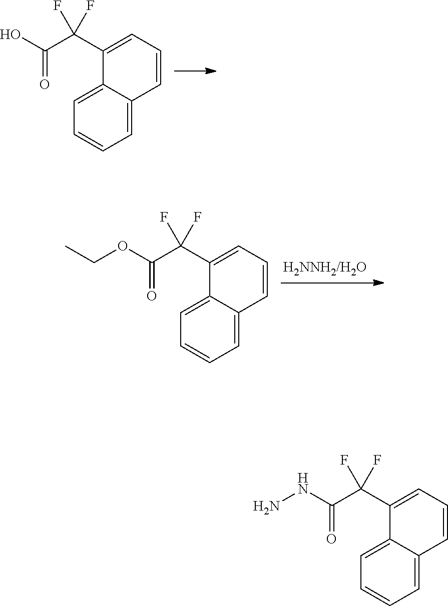 [SMILES: O=C(O)C(F)(F)c1cccc2ccccc12 ; CCOC(=O)C(F)(F)c1cccc2ccccc12 ; NNC(=O)C(F)(F)c1cccc2ccccc12]

2,2-difluoro-2-(naphthalen-1-yl)acetohydrazide was synthesized from 2,2-difluoro-2-(naphthalen-1-yl)acetic acid according to the procedures outlined for 2-(3,4-difluorophenyl)-2,2-difluoroacetohydrazide using the appropriate commercially available reagents and/or intermediates described elsewhere. LCMS m/z=237.1 [M+H]⁺.

### Synthesis of 2-(3-(tetrahydro-2H-pyran-4-yl)-1H-pyrazol-1-yl)propanoic acid

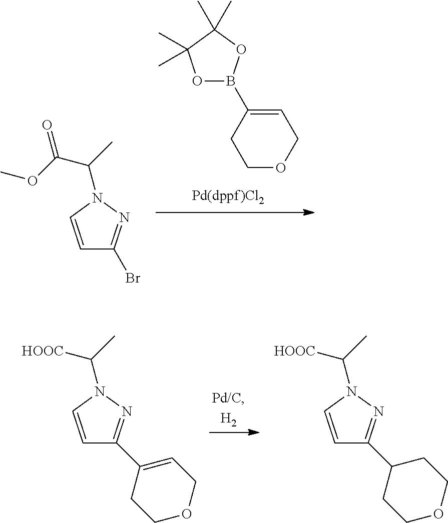 [SMILES: COC(=O)C(C)n1ccc(Br)n1 ; CC1(C)OB(C2=CCOCC2)OC1(C)C ; CC(C(=O)O)n1ccc(C2=CCOCC2)n1 ; CC(C(=O)O)n1ccc(C2CCOCC2)n1]

### Step 1: 2-(3-(3,6-dihydro-2H-pyran-4-yl)-1H-pyrazol-1-yl)propanoic acid

To a solution of methyl 2-(4-bromo-1H-pyrazol-1-yl)propanoate (300 mg, 1.29 mmol), K₂CO₃ (357 mg, 2.57 mmol) and Pd(dppf)Cl₂ (190 mg, 0.26 mmol) in a mixture of 1,4-dioxane (10 mL) and water (2 mL) under a N₂ atmosphere was added 2-(3,6-dihydro-2H-pyran-4-yl)-4,4,5,5-tetramethyl-1,3,2-dioxaborolane (405.6 mg, 1.93 mmol). The reaction mixture was heated at 100° C. overnight then was diluted with water (50 mL) and extracted with Et₂O (50 mL×2). The aqueous layer was collected and acidified to pH˜2 with 1M HCl then extracted with EtOAc (100 mL×2). The combined organic layers was washed with brine, dried over Na₂SO₄, filtered and concentrated to afford crude 2-(3-(3,6-dihydro-2H-pyran-4-yl)-1H-pyrazol-1-yl)propanoic acid (240 mg, 79.0%) as a yellow oil. LCMS m/z=223.2 [M+H]⁺; ¹H NMR (400 MHz, DMSO-d₆) δ 12.64 (s, 1H), 7.73 (d, J=2.4 Hz, 1H), 6.41 (d, J=2.4 Hz, 1H), 6.22 (dp, J=3.0, 1.6 Hz, 1H), 5.06 (q, J=7.4 Hz, 1H), 4.18 (q, J=2.8 Hz, 2H), 3.76 (t, J=5.6 Hz, 2H), 2.40 (dq, J=5.8, 3.0 Hz, 2H), 1.63 (d, J=7.4 Hz, 3H).

### Step 2: 2-(3-(tetrahydro-2H-pyran-4-yl)-1H-pyrazol-1-yl)propanoic acid

To a solution of 2-(3-(3,6-dihydro-2H-pyran-4-yl)-1H-pyrazol-1-yl)propanoic acid (140 mg, 0.62 mmol) in MeOH (2 mL) was added 10% Pd/C (20 mg). The reaction mixture was stirred under a H₂ atmosphere for 5 h then the catalyst was removed by filtration through celite and the filtrate concentrated to afford 2-(3-(tetrahydro-2H-pyran-4-yl)-1H-pyrazol-1-yl)propanoic acid (110 mg, 79%) which was used directly in the next step without further purification. LCMS m/z=225.2 [M+H]⁺; ¹H NMR (400 MHz, DMSO-d₆) δ 7.64 (d, J=2.4 Hz, 1H), 6.09 (d, J=2.4 Hz, 1H), 4.94 (q, J=7.2 Hz, 1H), 3.87 (ddd, J=11.6, 4.4, 2.0 Hz, 2H), 3.40 (td, J=11.6, 2.2 Hz, 4H), 2.77 (dq, J=11.6, 3.8 Hz, 1H), 1.77 (ddd, J=12.9, 4.2, 2.0 Hz, 2H), 1.65-1.49 (m, 4H).

### Synthesis of 2-(2-cyclopentyloxazol-4-yl)propanoic acid

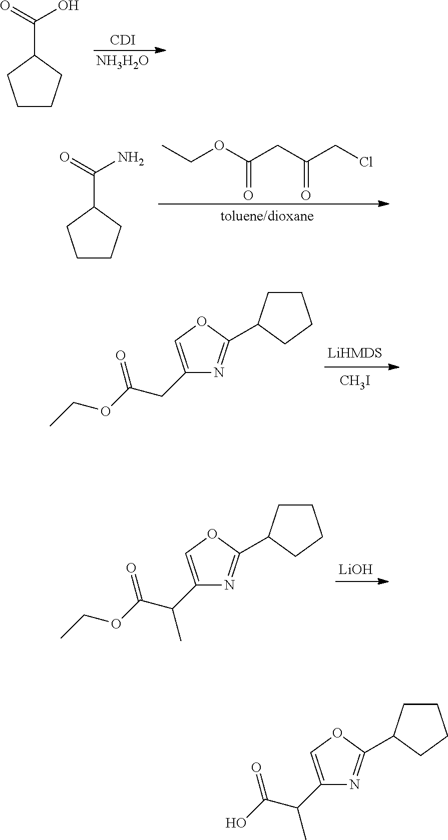

### Step 1: cyclopentanecarboxamide

To a solution of cyclopentanecarboxylic acid (5.0 g, 43.8 mmol) in EtOAc (66 mL) was added CDI (7.81 g, 48.18 mmol). The reaction was stirred for 1 hour then hydrazine hydrate (2.7 mL) was added and the reaction heated at 45° C. for 5 h. The reaction was diluted with water (50 mL) and extracted with EtOAc (150 mL×3). The combined organic layers were washed with brine, dried over Na₂SO₄, filtered and concentrated to afford cyclopentanecarboxamide (3.1 g, 63%) as colorless oil. LCMS m/z=113.2 [M+H]⁺.

### Step 2: ethyl 2-(2-cyclopentyloxazol-4-yl)acetate

To a solution of cyclopentanecarboxamide (400 mg, 23.53 mmol) in a mixture of toluene (4 mL) and 1,4-dioxane (4 mL) was added ethyl 4-chloro-3-oxobutanoate (640 mg, 3.89 mmol). The reaction was heated at 110° C. overnight then concentrated under reduced pressure. The residue obtained was purified by RP-column to afford ethyl 2-(2-cyclopentyloxazol-4-yl) acetate (420 mg, 53%) as a yellow oil. LCMS m/z=224.2 [M+H]⁺; ¹H NMR (400 MHz, Chloroform-d) δ 7.52 (d, J=1.2 Hz, 1H), 4.19 (q, J=7.0 Hz, 2H), 3.57 (d, J=1.0 Hz, 2H), 3.19 (p, J=8.0 Hz, 1H), 2.11-1.96 (m, 2H), 1.92-1.56 (m, 6H), 1.28 (t, J=7.2 Hz, 3H).

### Step 3: ethyl 2-(2-cyclopentyloxazol-4-yl)propanoate

To a solution of ethyl 2-(2-cyclopentyloxazol-4-yl) acetate (200 mg, 1.16 mmol) in anhydrous THF (3 mL) at −78° C. under a N₂ atmosphere was added LiHMDS (1.0 M in THF, 1.28 mL, 1.28 mmol) drop wise. The reaction mixture was stirred at −78° C. for 30 min then CH₃I (0.08 mL, 1.28 mmol) was added. The reaction was allowed to warm to room temperature and stirred for another 2 h then was diluted with water (10 mL) and extracted with EtOAc (150 mL×3). The combined organic layers were washed with sat. NH₄Cl (30 mL×2), dried over Na₂SO₄, filtered and concentrated. The residue was purified by RP-column to afford ethyl 2-(2-cyclopentyloxazol-4-yl) propanoate (45 mg, 16%) as a colorless oil. LCMS m/z=238.2 [M+H]⁻; ¹H NMR (400 MHz, Chloroform-d) δ 7.54 (s, 1H), 4.19 (tt, J=7.6, 4.4 Hz, 2H), 3.82 (q, J=7.2 Hz, 1H), 3.33 (q, J=8.2 Hz, 1H), 2.19-2.03 (m, 2H), 1.93-1.76 (m, 4H), 1.75-1.62 (m, 2H), 1.52 (d, J=7.4 Hz, 3H), 1.27 (t, J=7.0 Hz, 3H).

### Step 4: 2-(2-cyclopentyloxazol-4-yl)propanoic acid

To a solution of ethyl 2-(2-cyclopentyloxazol-4-yl)propanoate (65 mg, 0.273 mmol) in a mixture of THF, water and EtOH (4 mL/1 mL/1 mL) was added LiOH·H₂O (23 mg, 0.547 mmol). The reaction mixture was stirred at room temperature for 4 h then was diluted with water (10 mL) and extracted with ether (15 mL). The aqueous layer was collected and acidified to pH˜2 with 1M HCl then extracted with EtOAc (50 mL×3). The combined organic layers were washed with brine, dried over Na₂SO₄, filtered and concentrated to afford 2-(2-cyclopentyloxazol-4-yl)propanoic acid (45 mg, 79%) as a yellow solid which was used in the next step without purification. LCMS m/z=210.1 [M+H]⁻.

### Synthesis of 2-(2-cyclopentylthiazol-4-yl)propanoic acid

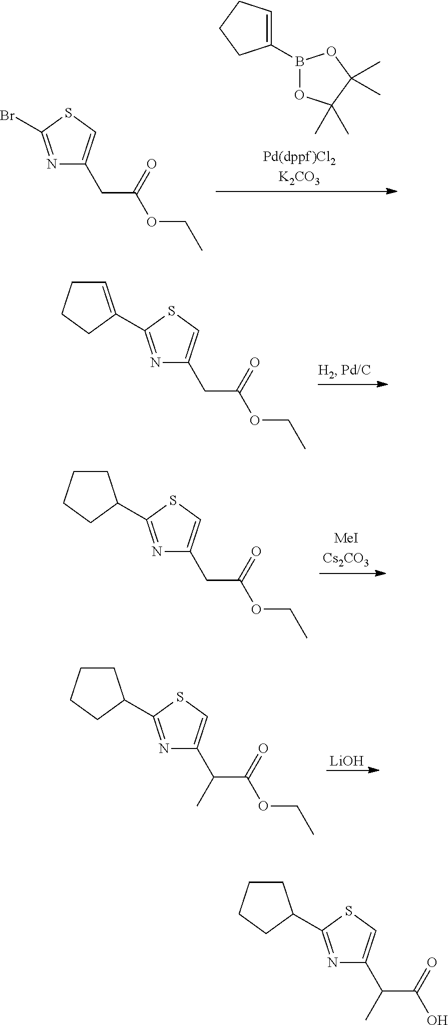

### Step 1: ethyl 2-(2-(cyclopent-1-en-1-yl)thiazol-4-yl)acetate

To a solution of ethyl 2-(2-bromothiazol-4-yl)acetate (2.0 g, 8.0 mmol) in a mixture of 1,4-dioxane (20 mL) and water (4 mL) was added Pd(dppf)Cl₂ (0.59 g, 0.8 mmol), 2-(cyclopent-1-en-1-yl)-4,4,5,5-tetramethyl-1,3,2-dioxaborolane (1.86 g, 9.6 mmol) and K₂CO₃ (2.21 g, 16.0 mmol). The reaction was heated under a N₂ atmosphere at 100° C. for 4 h then was diluted with water (30 mL) and extracted with EtOAc (80 mL×3). The combined organic layers were washed with brine, dried over Na₂SO₄, filtered and concentrated. The residue was purified by column chromatography on silica gel (eluent: Pet. Ether/EtOAc=20/1) to afford ethyl 2-(2-(cyclopent-1-en-1-yl)thiazol-4-yl)acetate (1.5 g, 79%) as a colorless oil. LCMS m/z=237.5 [M+H]⁺; ¹H NMR (400 MHz, Methanol-d4) δ 7.26 (s, 1H), 6.56-6.51 (m, 1H), 4.17 (q, J=7.2 Hz, 2H), 3.81 (s, 2H), 2.84-2.74 (m, 2H), 2.61-2.52 (m, 2H), 2.11-2.00 (m, 2H), 1.26 (t, J=7.2 Hz, 3H).

### Step 2: ethyl 2-(2-cyclopentylthiazol-4-yl)acetate

To a solution of ethyl 2-(2-(cyclopent-1-en-1-yl)thiazol-4-yl)acetate (1.5 g, 6.32 mmol) in MeOH (20 mL) was added 10% Pd/C (0.3 g). The mixture was stirred under a H₂ atmosphere for 14 hours then the catalyst was removed by filtration through celite and the filtrate concentrated to afford ethyl 2-(2-cyclopentylthiazol-4-yl)acetate (1.47 g, 98%) as colorless oil. LCMS m/z=240.1 [M+H]⁺.

### Step 3: ethyl 2-(2-cyclopentylthiazol-4-yl)propanoate

To a solution of ethyl 2-(2-cyclopentylthiazol-4-yl)acetate (1.4 g, 5.85 mmol) in DMF (15 mL) was added Cs₂CO₃ (2.29 g, 7.0 mmol) and CH₃I (1.25 g, 8.8 mmol). The reaction mixture was stirred at room temperature for 14 h then was diluted with water (40 mL) and extracted with EtOAc (50 mL×2). The combined organic layers were washed with brine, dried over Na₂SO₄, filtered and concentrated. The residue was purified by column chromatography on silica gel (eluent: Pet. Ether/EtOAc=40/1) to afford ethyl 2-(2-cyclopentylthiazol-4-yl)propanoate (880 mg, 59%) as colorless oil. LCMS m/z=253.5 [M+H]⁺; ¹H NMR (400 MHz, Methanol-d4) δ 7.15 (s, 1H), 4.15 (q, J=7.2 Hz, 2H), 3.93 (q, J=7.2 Hz, 1H), 3.51-3.37 (m, 1H), 2.25-2.10 (m, 2H), 1.87-1.68 (m, 6H), 1.51 (d, J=7.2 Hz, 3H), 1.22 (t, J=7.2 Hz, 3H).

### Step 4: 2-(2-cyclopentylthiazol-4-yl)propanoic acid

To a solution of ethyl 2-(2-cyclopentylthiazol-4-yl)propanoate (100 mg, 0.39 mmol) in a mixture of THF and water (2.0 mL/0.5 mL) was added LiOH (18.6 mg, 0.43 mmol). The reaction mixture was heated at 50° C. for 2 h then was diluted with water (10 mL) and extracted with ether (20 mL). The aqueous layer was collected and acidified to pH˜2 with 1M HCl then was extracted with EtOAc (30 mL×3). The combined organic layers were washed with brine, dried over Na₂SO₄, filtered and concentrated to afford 2-(2-cyclopentylthiazol-4-yl)propanoic acid (55 mg, 61%) as colorless oil. LCMS m/z=[M+H]^{+ 1}H NMR (400 MHz, CDCl₃) δ 6.96 (s, 1H), 3.87 (q, J=7.2 Hz, 1H), 3.51-3.40 (m, 1H), 2.25-2.15 (m, 2H), 1.87-1.68 (m, 6H), 1.62 (d, J=7.2 Hz, 3H).

### Synthesis of 2-(3,5-dichloro-1H-pyrazol-1-yl)propanoic acid

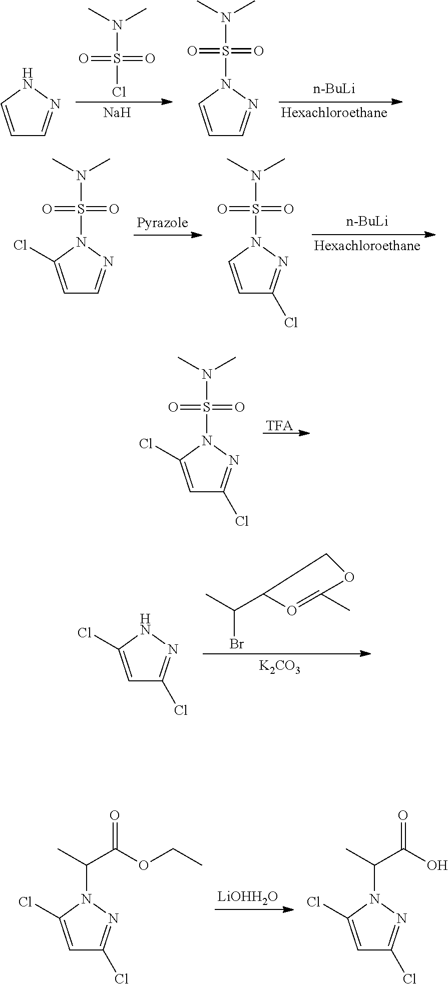

### Step 1: N, N-dimethyl-1H-pyrazole-1-sulfonamide

To a solution of 1H-pyrazole (3.0 g, 44.0 mmol) in THE (30 mL) at 0° C. was added NaH (2.64 g, 66.0 mmol). The reaction was stirred for 30 min then dimethylsulfamoyl chloride (9.5 g, 66.0 mmol) was added. The reaction was allowed to warm to room temperature and stirred for 4 h then was diluted with water (50 mL) and extracted with EtOAc (50 mL×2). The combined organic layers were washed with brine, dried over Na₂SO₄, filtered and concentrated. The residue obtained was purified by column chromatography on silica gel (eluent: Pet. Ether:EtOAc=1:1) to afford N,N-dimethyl-1H-pyrazole-1-sulfonamide (7.1 g, 92%) as a colorless oil. LCMS m/z=176.1 [M+H]⁺; ¹H NMR (400 MHz, DMSO-d₆) δ 8.25 (d, J=2.8 Hz, 1H), 7.90 (d, J=1.6 Hz, 1H), 6.57 (dd, J=2.8, 1.6 Hz, 1H), 2.84 (s, 6H).

### Step 2: 5-chloro-N,N-dimethyl-1H-pyrazole-1-sulfonamide

To a solution of N, N-dimethyl-1H-pyrazole-1-sulfonamide (4.0 g, 22.83 mmol) in anhydrous THF (49 mL) at −78° C. under a N₂ atmosphere was added n-BuLi (2.5 M in hexanes, 10.05 mL, 25.11 mmol) dropwise. The reaction was stirred for 30 min then a solution of hexachloroethane (5.94 g, 25.11 mmol) in anhydrous THE (50 mL) was added drop wise. The reaction was allowed to warm to room temperature and stirred for another 2 h then was diluted with saturated aq. NH₄Cl (150 mL) and extracted with EtOAc (100 mL×3). The combined organic layers were washed with brine, dried over Na₂SO₄, filtered and concentrated. The residue was purified by column chromatography on silica gel (eluent: Pet. Ether:EtOAc=5:1) to afford 5-chloro-N, N-dimethyl-1H-pyrazole-1-sulfonamide (3.2 g, 66%) as a yellow oil. LCMS m/z=210.1 [M+H]⁺; ¹H NMR (400 MHz, Chloroform-d) δ 7.61 (d, J=1.6 Hz, 1H), 6.32 (d, J=1.6 Hz, 1H), 3.07 (s, 6H).

### Step 3: 3-chloro-N,N-dimethyl-1H-pyrazole-1-sulfonamide

A mixture of 5-chloro-N,N-dimethyl-1H-pyrazole-1-sulfonamide (500 mg, 2.38 mmol) and pyrazole (16.2 mg, 0.238 mmol) was heated at 110° C. under a N₂ atmosphere overnight. The mixture was used directly in the next step. LCMS m/z=210.0 [M+H]⁺.

### Step 4: 3,5-dichloro-N,N-dimethyl-1H-pyrazole-1-sulfonamide

To a solution of 3-chloro-N, N-dimethyl-1H-pyrazole-1-sulfonamide (500 mg, 2.38 mmol) in anhydrous THE (10 mL) at −78° C. under a N₂ atmosphere was added n-BuLi (2.5 M in hexanes, 1.05 mL, 2.62 mmol) drop wise. The reaction mixture was stirred for 30 min then a solution of hexachloroethane (1.13 g, 4.77 mmol) in anhydrous THE (5 mL) was added dropwise. The reaction was allowed to warm to room temperature and stirred for another 4 h then was diluted with saturated aq. NH₄Cl (25 mL) and extracted with EtOAc (50 mL×3). The combined organic layers were washed with brine, dried over Na₂SO₄, filtered and concentrated. The residue was purified by reverse column to afford 3,5-dichloro-N,N-dimethyl-1H-pyrazole-1-sulfonamide (100 mg, 17%) as a yellow oil. LCMS m/z=244.1 [M+H]⁺; ¹H NMR (400 MHz, Chloroform-d) δ 6.28 (s, 1H), 3.10 (s, 6H).

### Step 5: 3,5-dichloro-1H-pyrazole

To a solution of 3, 5-dichloro-N, N-dimethyl-1H-pyrazole-1-sulfonamide (100 mg, 0.409 mmol) in DCM (2 mL) at 0° C. under a N₂ atmosphere was added TFA (0.01 mL). The reaction mixture was stirred at room temperature for 3 h then was diluted with water (10 mL) and extracted with Et₂O (50 mL×3). The combined organic layers were washed with brine, dried over Na₂SO₄, filtered and concentrated to afford 3,5-dichloro-1H-pyrazole (40 mg, 71%) as a yellow solid. LCMS m/z=137.1 [M+H]⁺; ¹HNMR (400 MHz, DMSO-d₆) δ 13.83 (s, 1H), 6.52 (s, 1H).

### Step 6: ethyl 2-(3,5-dichloro-1H-pyrazol-1-yl)propanoate

To a solution of 3, 5-dichloro-1H-pyrazole (40 mg, 0.29 mmol) in DMF (3 mL) was added K₂CO₃ (81 mg, 0.58 mmol) and ethyl 2-bromopropanoate (79.0 mg, 0.44 mmol). The reaction mixture was stirred at room temperature for 4 h then diluted with water (10 mL) and extracted with EtOAc (20 mL×3). The combined organic layers were washed with brine, dried over Na₂SO₄, filtered and concentrated to afford crude ethyl 2-(3,5-dichloro-1H-pyrazol-1-yl)propanoate (70.0 mg, quant.) as yellow oil. ¹H NMR (400 MHz, Chloroform-d) δ 6.20 (d, J=0.8 Hz, 1H), 5.07 (qd, J=7.2, 1.0 Hz, 1H), 4.29-4.04 (m, 2H), 1.81 (dd, J=7.2, 1.0 Hz, 3H), 1.24 (td, J=7.2, 0.8 Hz, 4H).

### Step 7: 2-(3,5-dichloro-1H-pyrazol-1-yl)propanoic acid

To a solution of ethyl 2-(3,5-dichloro-1H-pyrazol-1-yl)propanoate (70 mg, 0.295 mmol) in a mixture of THF, water and EtOH (4 mL/1 mL/1 mL) was added LiOHH₂O (25 mg, 0.59 mmol). The reaction was stirred at room temperature for 4 h then was diluted with water (10 mL) and extracted with ether (15 mL). The aqueous layer was collected and acidified to pH˜2 with 1M HCl then was extracted with EtOAc (50 mL×3). The combined organic layers were washed with brine, dried over Na₂SO₄, filtered and concentrated to afford 2-(3,5-dichloro-1H-pyrazol-1-yl)propanoic acid (45 mg, 73%) as a yellow solid which was used directly in the next step. LCMS m/z=209.0 [M+H]⁺; ¹H NMR (400 MHz, DMSO-d₆) δ 6.65 (s, 1H), 5.24 (q, J=7.2 Hz, 1H), 1.64 (d, J=7.2 Hz, 3H).

### Synthesis of 1-(3,4-dichlorophenyl)-3-hydroxycyclobutane-1-carbohydrazide

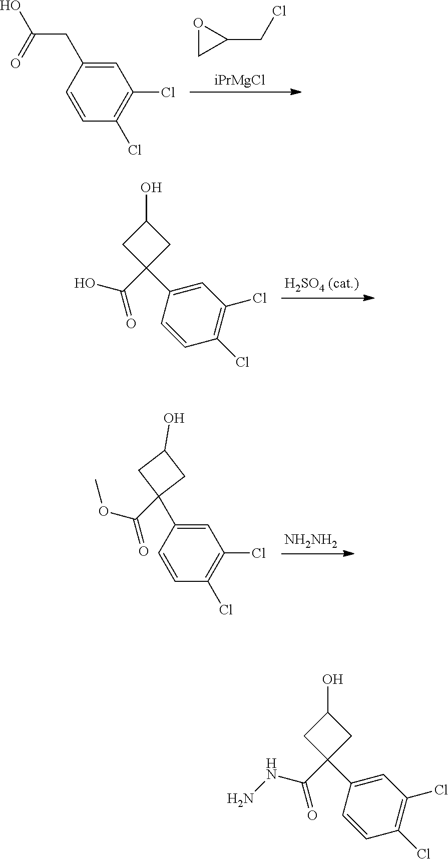

### Step 1: 1-(3,4-dichlorophenyl)-3-hydroxycyclobutane-1-carboxylic acid

To a solution of 2-(3,4-dichlorophenyl)acetic acid (3.0 g, 14.6 mmol) in dry THF (15 mL) at 0° C. was added iPrMgCl (14.6 mL, 29.2 mmol) and the reaction stirred for 10 min. epichlorohydrin (2.44 g, 26.4 mmol) was added dropwise and the reaction allowed to warm to room temperature and stirred for 45 min. Additional iPrMgCl (14.6 ml, 29.2 mmol) was added dropwise over 20 min and the reaction heated at 60° C. for 14 h then was diluted with water (50 mL). The pH of the aqueous solution was adjusted the pH to 1 with 1 M HCl, and the aqueous extracted with EtOAc (50 mL×3). The combined organic layers were washed with water and brine, dried over Na₂SO₄, filtered and concentrated. The residue was triturated with ether to afford 1-(3,4-dichlorophenyl)-3-hydroxycyclobutane-1-carboxylic acid (1.21 g, 31%) as a white solid. LCMS m/z=258.8 [M−H]⁻; ¹H NMR (400 MHz, DMSO-d6) δ 7.64-7.54 (m, 2H), 7.36 (dd, J=8.4, 2.4 Hz, 1H), 3.92-3.81 (M, 1H), 2.79-2.70 (m, 2H), 2.55-2.50 (m, 2H).

### Step 2: methyl 1-(3,4-dichlorophenyl)-3-hydroxycyclobutane-1-carboxylate

To a solution of 1-(3,4-dichlorophenyl)-3-hydroxycyclobutane-1-carboxylic acid (1.21 g, 4.6 mmol) in CH₃OH (15 mL) was added a drop of H₂SO₄. The mixture was heated at 80° C. for 3 h then diluted with aqueous NaHCO₃ (20 mL) and extracted with EtOAc (40 mL×2). The combined organic layers were washed with water, brine, dried over Na₂SO₄, filtered and concentrated to afford methyl 1-(3,4-dichlorophenyl)-3-hydroxycyclobutane-1-carboxylate (1.1 g, 86%) as a white solid. ¹H NMR (400 MHz, Methanol-d4) δ 7.57-7.46 (m, 2H), 7.33 (dd, J=8.4, 2.4 Hz, 1H), 4.12-3.99 (m, 1H), 3.64 (s, 3H), 2.91-2.78 (m, 2H), 2.72-2.61 (m, 2H).

### Step 3: 1-(3,4-dichlorophenyl)-3-hydroxycyclobutane-1-carbohydrazide

To a solution of methyl 1-(3,4-dichlorophenyl)-3-hydroxycyclobutane-1-carboxylate (1.02 g, 3.7 mmol) in MeOH (4 mL) was added hydrazine hydrate (98%, 2 mL). The mixture was heated at 110° C. for 1.5 h in the microwave. The mixture was diluted with water (20 mL) and extracted with EtOAc (50 mL×3). The combined organic layers were washed with water and brine, dried over Na₂SO₄, filtered and concentrated to afford 1-(3,4-dichlorophenyl)-3-hydroxycyclobutane-1-carbohydrazide (0.49 g, 48%) as a white solid. LCMS m/z=276.0[M+H]⁺.

### Synthesis of 1-amino-3-(4-chloro-3-(trifluoromethyl)phenyl)propan-2-ol

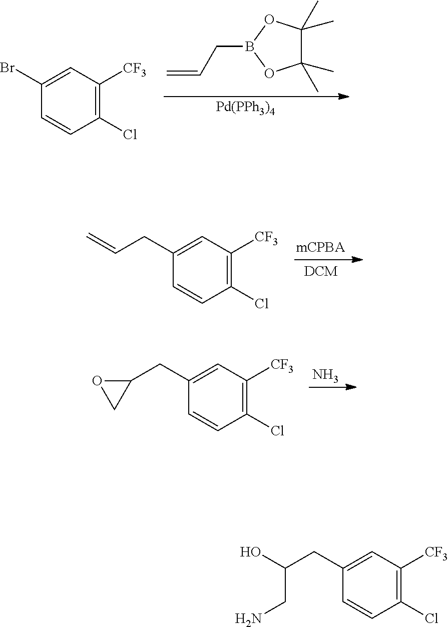

### Step 1: 4-allyl-1-chloro-2-(trifluoromethyl)benzene

To a solution of 4-bromo-1-chloro-2-(trifluoromethyl)benzene (2.0 g, 7.71 mmol) in a mixture of DME (16 mL) and H₂O (4 mL) under N₂ atmosphere was added 2-allyl-4,4,5,5-tetramethyl-1,3,2-dioxaborolane (1.5 g, 9.25 mmol), K₂CO₃ (2.1 g, 15.4 mmol) and Pd(PPh₃)₄ (890 mg, 0.07 mmol). The reaction mixture was stirred at 90° C. overnight then was diluted with water (30 mL) and extracted with EtOAc (75 mL×3). The combined organic layers were washed with NH₄Cl aq, dried over Na₂SO₄, filtered and concentrated. The residue was purified by column on silica gel (eluent: pure Pet. Ether) to afford 4-allyl-1-chloro-2-(trifluoromethyl)benzene (890 mg, 52%). ¹H NMR (400 MHz, Methanol-d4) δ 7.60-7.56 (m, 1H), 7.53-7.40 (m, 2H), 6.03-5.91 (m, 1H), 5.15-5.07 (m, 2H), 3.45 (d, J=6.8 Hz, 2H).

### Step 2: 2-(4-chloro-3-(trifluoromethyl)benzyl)oxirane

To a solution of 4-allyl-1-chloro-2-(trifluoromethyl)benzene (800 mg, 3.63 mmol) in DCM (6 mL) was added m-CPBA (688 mg, 3.9 mmol). The mixture was stirred at room temperature for 3 days then was diluted with water (30 mL) and extracted with DCM (70 mL×3). The combined organic layers were dried over Na₂SO₄, filtered and concentrated to afford the crude 2-(4-chloro-3-(trifluoromethyl)benzyl)oxirane (780 mg, 91%) as a yellow oil. ¹H NMR (400 MHz, DMSO-d₆) δ 7.80-7.65 (m, 2H), 7.64-7.57 (m, 1H), 3.22-3.12 (m, 1H), 3.01-2.93 (m, 1H), 2.89-2.80 (m, 1H), 2.74 (t, J=4.8 Hz, 1H), 2.58-2.53 (m, 1H).

### Step 3: 1-amino-3-(4-chloro-3-(trifluoromethyl)phenyl)propan-2-ol

2-(4-chloro-3-(trifluoromethyl)benzyl)oxirane (700 mg, 2.96 mmol) in a solution of NH₃ in MeOH (7 M, 10 mL) in sealed reaction vessel was heated at 70° C. for 2 h. The reaction was diluted with water (30 mL) and extracted with EtOAc (50 mL×3). The combined organic layers was washed with brine, dried over Na₂SO₄, filtered and concentrated. The residue was purified by reverse phase column to afford 1-amino-3-(4-chloro-3-(trifluoromethyl)phenyl)propan-2-ol (660 mg, 88%) as yellow oil. LCMS m/z=254.1 [M+H]⁺; ¹H NMR (400 MHz, DMSO-d₆) δ 7.93-7.45 (m, 6H), 3.87 (tt, J=8.2, 4.0 Hz, 1H), 2.98-2.57 (m, 4H).

### Synthesis of 2-(3,4-dichlorophenyl)-2-methylpropanehydrazide

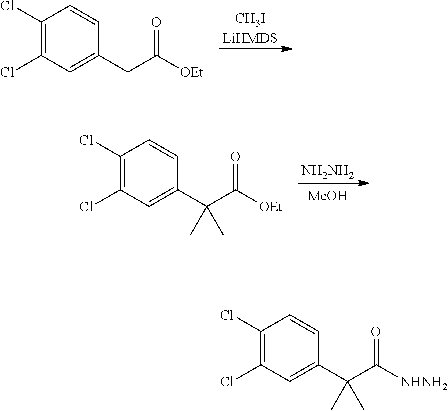 [SMILES: "MeOH" ; CCOC(=O)Cc1ccc(Cl)c(Cl)c1 ; "CH3ILiHMDS" ; CCOC(=O)C(C)(C)c1ccc(Cl)c(Cl)c1 ; "NH2NH2" ; CC(C)(C(=O)NN)c1ccc(Cl)c(Cl)c1]

### Step 1: ethyl 2-(3,4-dichlorophenyl)-2-methylpropanoate

To a solution of ethyl 2-(3,4-dichlorophenyl)acetate (500 mg, 2.15 mmol) in anhydrous THF (3 mL) at −78° C. under a N₂ atmosphere was added LiHMDS (2.5 M in hexanes, 8.5 mL, 8.5 mmol) dropwise. The reaction mixture was stirred for 30 min then CH₃I (3.0 g, 21.45 mmol) was added. The reaction was allowed to warm to room temperature and stirred for another 2 h then was diluted with EtOAc (250 mL) and washed with sat. NH₄Cl (30 mL×2). The combined organic layers was washed with brine, dried over Na₂SO₄, filtered and concentrated. The residue obtained was purified by prep-TLC (eluent: Pet. Ether:EtOAc=30:1) to afford ethyl 2-(3,4-dichlorophenyl)-2-methylpropanoate (480 mg, 87%) as a colorless oil. LCMS m/z=261.1 [M+H]⁺; ¹H NMR (400 MHz, Chloroform-d) δ 7.49-7.33 (m, 2H), 7.21-7.17 (m, 1H), 4.14 (q, J=7.2 Hz, 2H), 1.56 (s, 6H), 1.20 (t, J=7.2 Hz, 3H).

### Step 2: 2-(3,4-dichlorophenyl)-2-methylpropanehydrazide

To a solution of ethyl 2-(3,4-dichlorophenyl)-2-methylpropanoate (200 mg, 0.77 mmol) in MeOH (3 mL) was added hydrazine hydrate (98%, 0.3 mL) and the reaction stirred at room temperature overnight. The mixture was diluted with water (50 mL) and extracted with DCM:MeOH=5:1 (75 mL×3). The combined organic layers were dried over Na₂SO₄, filtered and concentrated to afford 2-(3,4-dichlorophenyl)-2-methylpropanehydrazide (80 mg, 43%) as a white solid. LCMS m/z=247.1 [M+H]⁺; ¹H NMR (400 MHz, DMSO-d₆) δ 8.89 (s, 1H), 7.58 (d, J=8.0 Hz, 1H), 7.51 (d, J=4.0 Hz, 1H), 7.30 (dd, J=4.0 Hz, 1H), 4.48-4.07 (m, 2H), 1.47 (s, 6H).

### Synthesis of ((tert-butoxycarbonyl)amino)alanine

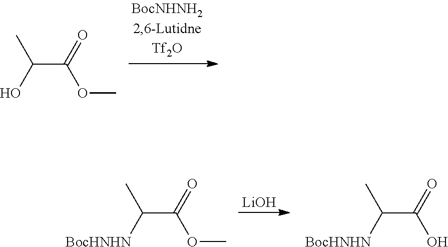

### Step 1: tert-butyl 2-(1-methoxy-1-oxopropan-2-yl)hydrazine-1-carboxylate

To a solution of methyl 2-hydroxypropanoate (2.0 g, 19.22 mmol) in DCM (10.0 mL) at 0° C. under an atmosphere of nitrogen was added 2,6-dimethylpyridine (5.2 mL, 44.20 mmol) and trifluoromethanesulfonic anhydride (3.6 mL, 21.14 mmol). The mixture was stirred at 0° C. for 20 min then a solution of tert-Butyl carbazate (2.54 g, 19.2 mmol) in DCM (5.0 mL) was added dropwise over 20 min. The reaction was stirred for 6 hours at 0° C. then was diluted with water (30 mL) and extracted with DCM (60 mL×3). The combined organic layers were washed with brine, dried over Na₂SO₄, filtered and concentrated. The residue was purified by column chromatography on silica gel (eluent: Pet.Ether/EtOAc=5/1) to afford tert-butyl 2-(1-methoxy-1-oxopropan-2-yl)hydrazine-1-carboxylate (2.1 g, 50%) as a yellow oil. ¹H NMR (400 MHz, CDCl₃) δ 3.73 (s, 3H), 3.72-3.65 (m, 1H), 1.44 (s, 9H), 1.31 (d, J=7.2 Hz, 3H).

### Step 2: ((tert-butoxycarbonyl)amino)alanine

To a solution of tert-butyl 2-(1-methoxy-1-oxopropan-2-yl)hydrazine-1-carboxylate (200 mg, 0.92 mmol) in a mixture of THF and water (2 mL/1 mL) was added lithium hydroxide monohydrate (79 mg, 1.84 mmol). The reaction mixture was stirred at room temperature for 2 h, then was diluted with water (10 mL) and extracted with ether (15 mL×2). The aqueous layer was collected and acidified to pH˜1 with 1M HCl then was extracted with EtOAc (30 mL×3). The combined organic layers were washed with brine, dried over Na₂SO₄, filtered and concentrated to afford ((tert-butoxycarbonyl)amino)alanine (62 mg, 33%) as a yellow solid which was used directly in the next step. ¹H NMR (400 MHz, CDCl₃) δ 3.75 (s, 1H), 1.46 (s, 9H), 1.37 (d, J=7.2 Hz, 3H).

### Synthesis of 2-(2-cyclopentyloxazol-4-yl)-2-methylpropanoic acid

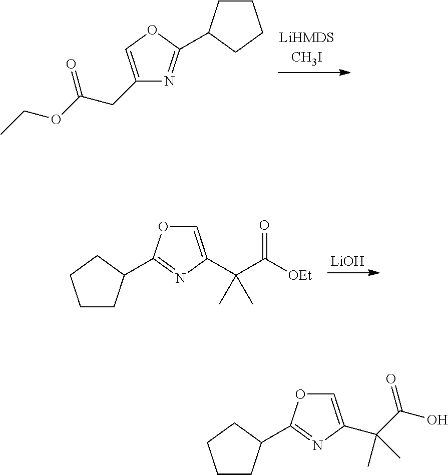

### Step 1: ethyl 2-(2-cyclopentyloxazol-4-yl)-2-methylpropanoate

To a solution of ethyl 2-(2-cyclopentyloxazol-4-yl) acetate (200 mg, 1.16 mmol) in anhydrous THF (3 mL) at −78° C. under a N₂ atmosphere was added LiHMDS (1.0 M in THF, 2.56 mL, 2.56 mmol) drop wise. The reaction mixture was stirred at −78° C. for 30 min then CH₃I (0.4 mL, 6.4 mmol) was added. The reaction was allowed to warm to room temperature and stirred for another 2 h then was diluted with water (20 mL) and extracted with EtOAc (50 mL×3). The combined organic layers were washed with sat. NH₄Cl (10 mL×2), dried over Na₂SO₄, filtered and concentrated. The residue was purified by reverse phase column to afford ethyl 2-(2-cyclopentyloxazol-4-yl)-2-methylpropanoate (45 mg, 16%) as a colorless oil. LCMS m/z=252.1 [M+H]⁺; ¹H NMR (400 MHz, Chloroform-d) δ 7.35 (s, 1H), 4.14 (q, J=7.2 Hz, 2H), 3.19 (p, J=8.2 Hz, 1H), 2.08-1.94 (m, 2H), 1.89-1.56 (m, 6H), 1.51 (s, 6H), 1.21 (t, J=7.2 Hz, 3H).

### Step 2: 2-(2-cyclopentyloxazol-4-yl)-2-methylpropanoic acid

To a solution of ethyl 2-(2-cyclopentyloxazol-4-yl)-2-methylpropanoate (100 mg, 0.398 mmol) in a mixture of THF, water and EtOH (4 mL/1 mL/1 mL) was added lithium hydroxide monohydrate (34 mg, 0.796 mmol). The reaction mixture was stirred at room temperature for 4 h then was diluted with water (10 mL) and extracted with ether (15 mL). The aqueous layer was collected and acidified to pH˜2 with 1M HCl then extracted with EtOAc (50 mL×3). The combined organic layers were washed with brine, dried over Na₂SO₄, filtered and concentrated to afford 2-(2-cyclopentyloxazol-4-yl)propanoic acid (45 mg, 79%) as a white solid which was used directly in the next step. LCMS m/z=224.1 [M+H]⁺.

### Synthesis of ethyl 2-(3,4-dichlorophenyl)acetimidate hydrochloride

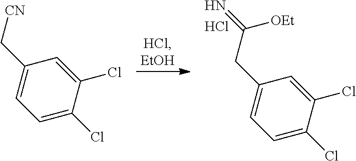 [SMILES: N#CCc1ccc(Cl)c(Cl)c1 ; Cl ; CCOC(=N)Cc1ccc(Cl)c(Cl)c1]

### Step 1: ethyl 2-(3,4-dichlorophenyl)acetimidate hydrochloride

HCl gas was bubbled through a solution of 2-(3,4-dichlorophenyl)acetonitrile (1.12 g, 6 mmol) in CHCl₃ (5.0 mL) and ethanol (0.35 mL) for 1 h. The reaction was stirred at 0° C. for 14 h then the solvent was removed and the solid obtained resuspended in Et₂O (15 mL) and collected by filtration to afford ethyl 2-(3,4-dichlorophenyl)acetimidate hydrochloride (0.7 g, 50%) as a white solid. ¹H NMR (400 MHz, DMSO-d6) δ 7.71 (d, J=2.0 Hz, 1H), 7.66 (d, J=8.4 Hz, 1H), 7.39 (dd, J=10.4, 2.0 Hz, 1H), 4.41 (q, J=7.2 Hz, 2H), 4.11-4.04 (m, 2H), 1.28 (t, J=6.8 Hz, 3H).

### Synthesis of 1-amino-3-(4-chlorophenyl)propan-2-ol

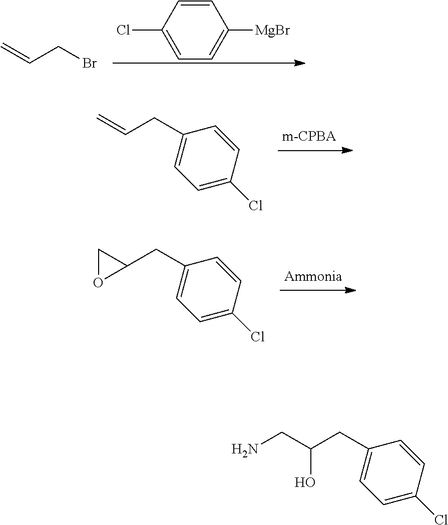

### Step 1: 1-allyl-4-chlorobenzene

To a solution of allyl bromide (1.0 g, 8.26 mmol) in anhydrous THE (10.0 mL) at 0° C. was added (4-chlorophenyl)magnesium bromide (1.0 mol/L in THF, 4.13 mL, 4.13 mmol). The reaction was stirred at room temperature for 2 h then was quenched with saturated aqueous NH₄Cl and extracted with EtOAc three times. The combined organic layers were washed with 1 M HCl, brine, dried over Na₂SO₄ and concentrated. The residue was purified by column chromatography on silica gel (eluent: pure Pet.Ether) to afford 1-allyl-4-chlorobenzene (1.16 g, 92%). ¹H NMR (400 MHz, Methanol-d4) δ 7.35-7.14 (m, 4H), 6.11-5.84 (m, 1H), 5.10 (m, J=6.1, 1.6 Hz, 1H), 5.07 (m, J=1.5 Hz, 1H), 3.38 (m, J=6.8, 1.5 Hz, 2H).

### Step 2: 2-(4-chlorobenzyl)oxirane

To a solution of 1-allyl-4-chlorobenzene (1.0 g, 6.55 mmol) in DCM (20 mL) at 0° C. was added m-CPBA (1.24 g, 7.21 mmol) and the mixture stirred at room temperature for 3 days. The reaction was quenched with saturated aqueous Na₂CO₃ and extracted with chloroform three times. The combined organic layers were dried over Na₂SO₄, filtered and concentrated to give 2-(4-chlorobenzyl)oxirane (840 mg, 76%). ¹H NMR (400 MHz, Methanol-d4) δ 7.27 (q, J=8.5 Hz, 4H), 3.12 (dtd, J=6.7, 4.2, 2.7 Hz, 1H), 2.88 (dd, J=14.6, 4.7 Hz, 1H), 2.81-2.73 (m, 2H), 2.54 (dd, J=5.0, 2.7 Hz, 1H).

### Step 3: 1-amino-3-(4-chlorophenyl)propan-2-ol

A solution of 2-(4-chlorobenzyl)oxirane (840 mg, 4.98 mmol) in a solution of ammonia in methanol (7 M, 10 mL) was heated at 100° C. in sealed tube for 2 h. The solvent was removed and the residue obtained purified by RP-column to give 1-amino-3-(4-chlorophenyl)propan-2-ol (554 mg, 60%) as a white solid. LCMS m/z=186.2 [M+H]⁺; ¹H NMR (400 MHz, Methanol-d4) δ 7.38-7.14 (m, 4H), 3.96 (dtd, J=9.6, 6.5, 3.0 Hz, 1H), 2.99 (dd, J=12.8, 3.0 Hz, 1H), 2.84-2.72 (m, 3H).

### Synthesis of 1-amino-3-(3,4-dichlorophenyl)propan-2-ol

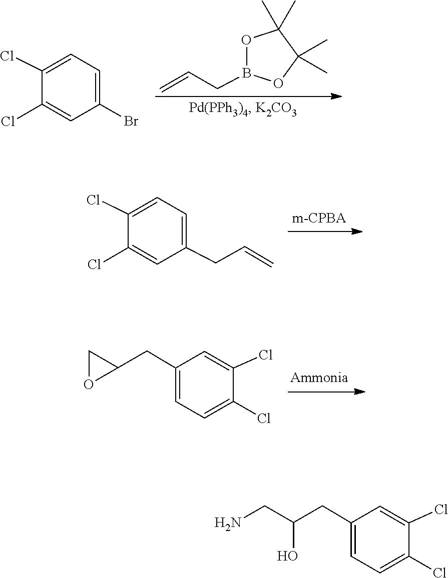

### Step 1: 4-allyl-1,2-dichlorobenzene

To a solution of 4-bromo-1,2-dichlorobenzene (5.0 g, 22.13 mmol) in a mixture of DME (160 mL) and water (40 mL) was added 2-allyl-4,4,5,5-tetramethyl-1,3,2-dioxaborolane (3.7 g, 22.13 mmol), Pd(PPh₃)₄ (5.1 g, 44.27 mmol) and K₂CO₃ (6.1 g, 4.43 mmol). The resulting mixture was heated at 90° C. under a N₂ atmosphere overnight then was diluted with water, extracted with EtOAc three times. The combined organic layers were washed with brine, dried over Na₂SO₄ and concentrated. The residue was purified by column chromatography on silica gel (eluent: pure Pet.Ether) to afford 4-allyl-1,2-dichlorobenzene (2.67 g, 65%). ¹H NMR (400 MHz, Methanol-d4) δ 7.41 (d, J=8.3 Hz, 1H), 7.34 (d, J=2.0 Hz, 1H), 7.12 (dd, J=8.3, 2.1 Hz, 1H), 5.94 (m, J=17.5, 9.7, 6.7 Hz, 1H), 5.09 (dd, J=13.6, 2.1 Hz, 2H), 3.37 (d, J=6.7 Hz, 2H).

### Step 2: 2-(3,4-dichlorobenzyl)oxirane

To a solution of 4-allyl-1,2-dichlorobenzene (2.7 g, 14.27 mmol) in DCM (50 mL) at 0° C. was added m-CPBA (2.71 g, 15.70 mmol). Then the reaction was heated at 90° C. overnight then was quenched with saturated aqueous Na₂CO₃ and extracted with chloroform three times. The combined organic layers were dried over Na₂SO₄, filtered and concentrated to give 2-(3,4-dichlorobenzyl)oxirane (2.9 g, quant). ¹H NMR (400 MHz, Chloroform-d) δ 7.38 (d, J=8.3 Hz, 1H), 7.36 (d, J=2.1 Hz, 1H), 7.10 (dd, J=8.2, 2.0 Hz, 1H), 3.12 (tdd, J=6.3, 4.2, 2.6 Hz, 1H), 2.90-2.76 (m, 3H), 2.52 (dd, J=4.9, 2.6 Hz, 1H).

### Step 3: 1-amino-3-(3,4-dichlorophenyl)propan-2-ol

A solution of 2-(3,4-dichlorobenzyl)oxirane (2.9 g, 14.28 mmol) in a solution of ammonia in methanol (7 M, 30 mL) was heated at 100° C. in sealed tube for 1 h. The solvent was removed and the residue obtained was purified by RP-column to give 1-amino-3-(3,4-dichlorophenyl)propan-2-ol (2.3 g, 73%) as a yellow oil. ¹H NMR (400 MHz, Methanol-d4) δ 7.46 (d, J=2.0 Hz, 1H), 7.45 (d, J=4.1 Hz, 1H), 7.19 (dd, J=8.2, 2.0 Hz, 1H), 3.97 (dddd, J=9.6, 8.0, 5.2, 3.1 Hz, 1H), 3.03 (dd, J=12.8, 2.9 Hz, 1H), 2.84-2.75 (m, 3H).

### Synthesis of 2-(3-hydroxy-5-methyl-1H-pyrazol-1-yl)propanoic acid

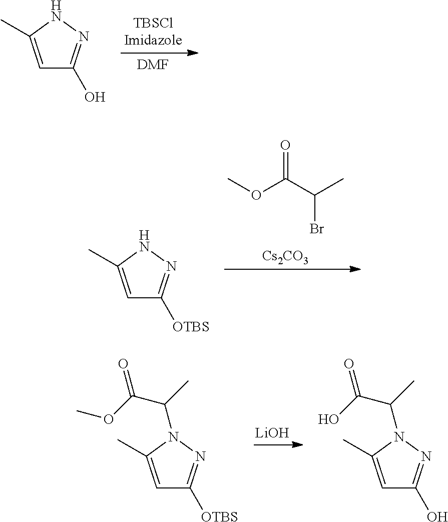

### Step 1: 3-((tert-butyldimethylsilyl)oxy)-5-methyl-1H-pyrazole

To a solution of 5-methyl-1H-pyrazol-3-ol (2 g, 20.6 mmol, 1.0 eq) in DMF at 0° C. was added TBSCl (3.7 g, 24.7 mmol, 1.2 eq) and imidazole (1.6 g, 24.7 mmol, 1.2 eq). The resulting mixture was stirred at room temperature overnight then was diluted with water (100 mL) and extracted with EtOAc (150 mL×2). The combined organic layers were washed with brine, dried over Na₂SO₄, filtered and concentrated to afford the 3-((tert-butyldimethylsilyl)oxy)-5-methyl-1H-pyrazole (1 g, 23%) as a yellow oil.

### Step 2: methyl 2-(3-((tert-butyldimethylsilyl)oxy)-5-methyl-1H-pyrazol-1-yl)propanoate

To a solution of 3-((tert-butyldimethylsilyl)oxy)-5-methyl-1H-pyrazole (1.0 g, 10.3 mmol, 1.0 eq) in DMF (5 mL) was added added methyl 2-bromopropanoate (1.7 g, 10.3 mmol, 1.0 eq) and Cs₂CO₃ (6.7 g, 20.6 mmol, 2.0 eq.). The mixture was stirred at room temperature overnight then was diluted with water (100 mL) and extracted with EtOAc (150 mL×2). The combined organic layers were washed with brine, dried over Na₂SO₄, filtered and concentrated. The residue was purified by column chromatography on silica gel (eluent: Pet. Ether:EtOAc=10:1 to 5:1) to afford methyl 2-(3-((tert-butyldimethylsilyl)oxy)-5-methyl-1H-pyrazol-1-yl)propanoate (800 mg, 57%) as a yellow oil. LCMS m/z=299.1 [M+H]⁺; ¹H NMR (400 MHz, CD₃OD) δ 5.43 (d, J=0.9 Hz, 1H), 4.97 (q, J=7.1 Hz, 1H), 3.68 (s, 3H), 2.20 (d, J=0.7 Hz, 3H), 1.64 (d, J=7.1 Hz, 3H), 0.97 (s, 9H), 0.21 (d, J=1.2 Hz, 6H).

### Step 3: 2-(3-hydroxy-5-methyl-1H-pyrazol-1-yl)propanoic acid

To a solution of methyl 2-(3-((tert-butyldimethylsilyl)oxy)-5-methyl-1H-pyrazol-1-yl)propanoate (500 mg, 1.6 mmol, 1.0 eq.) in THF (5 mL) was added 10% aq. NaOH solution (5 mL). The mixture was stirred at room temperature for 4 h then the THF was removed and the pH of the aqueous solution adjusted to pH=2 with 1 M HCl. The mixture was extracted with EtOAc (100 mL×2) and the combined organic layers were washed with brine, dried over Na₂SO₄, filtered and concentrated to afford crude 2-(3-hydroxy-5-methyl-1H-pyrazol-1-yl)propanoic acid (320 mg, 57%) as yellow solid. LCMS m/z=171.1 [M+H]⁺.

### Synthesis of 2-(1-(tetrahydro-2H-pyran-2-yl)-1H-pyrazol-3-yl)propanoic acid

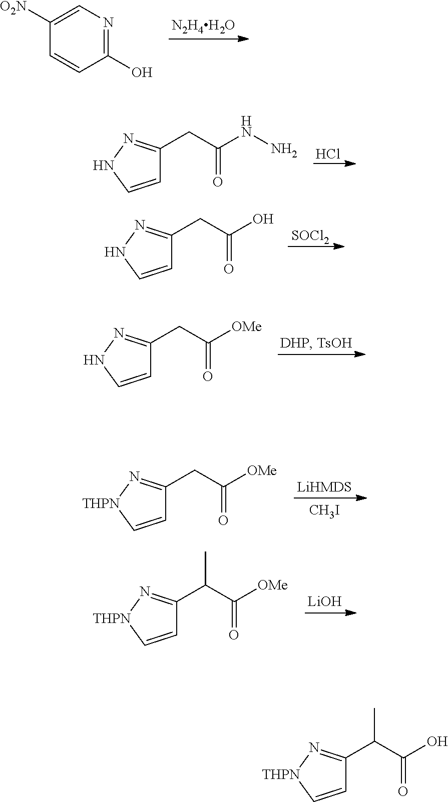

### Step 1: 2-(1H-pyrazol-3-yl)acetohydrazide

A mixture of 5-nitropyridin-2-ol (100 mg, 0.7 mmol) in hydrazine hydrate (714 mg, 14 mmol) was heated at 100° C. for 2 h. The mixture was concentrated under vacuum to give 2-(1H-pyrazol-3-yl)acetohydrazide (100 mg, quant.) as a yellow solid. LCMS m/z=141.1 [M+H]⁺

### Step 2: 2-(1H-pyrazol-3-yl)acetic acid

A mixture of 2-(1H-pyrazol-3-yl)acetohydrazide (100 mg, 0.7 mmol) in 10% aq. HCl (2 mL) was heated at 100° C. for 4 h. The mixture was concentrated under vacuum to give 2-(1H-pyrazol-3-yl)acetic acid (90 mg, quant.) as a yellow solid. LCMS m/z=127.1[M+H]⁺

### Step 3: methyl 2-(1H-pyrazol-3-yl)acetate

To a solution of 2-(1H-pyrazol-3-yl)acetohydrazide (45 mg, 0.35 mmol) in MeOH (2 mL) was added SOCl₂ (0.2 mL) dropwise. The resulting mixture was stirred at 65° C. for 4 h then the solvent was removed under vacuum. The residue was diluted with aq. NaHCO₃ then the solvent removed again. The mixture was re-suspended in a mixture of DCM and MeOH (10/1, 20 mL×2) and the solids removed by filtration. The filtrate was concentrated to give methyl 2-(1H-pyrazol-3-yl)acetate (40 mg, quant.) as a yellow solid. LCMS m/z=141.1 [M+H]⁺; ¹H NMR (400 MHz, CD₃OD) δ 7.55 (d, J=2.2 Hz, 1H), 6.25 (d, J=2.1 Hz, 1H), 3.71 (s, 2H), 3.70 (d, J=1.3 Hz, 3H).

### Step 4: methyl 2-(1-(tetrahydro-2H-pyran-2-yl)-1H-pyrazol-3-yl)acetate

To a solution of methyl 2-(1H-pyrazol-3-yl)acetate (50 mg, 0.35 mmol) in a mixture of EtOAc/DMF (1 mL/0.2 mL) was added TsOH (7 mg, 35 umol) and the reaction heated at 60° C. overnight. The solvent was removed under reduced pressure and the residue obtained purified by RP-column to afford methyl 2-(1-(tetrahydro-2H-pyran-2-yl)-1H-pyrazol-3-yl)acetate (36 mg, 72%) as a yellow solid. LCMS m/z=225.1 [M+H]⁺.

### Step 5: methyl 2-(1-(tetrahydro-2H-pyran-2-yl)-1H-pyrazol-3-yl)propanoate

To a solution of methyl 2-(1-(tetrahydro-2H-pyran-2-yl)-1H-pyrazol-3-yl)acetate (200 mg, 0.89 mmol) in anhydrous THF (1 mL) at 0° C. was added LiHMDS (1 M in THF, 1 mL) and the reaction stirred for 30 min. CH₃I (253 mg, 1.8 mmol, 2.0 eq.) was added and the reaction allowed to warm to room temperature and stirred a further 2 h. The mixture was concentrated and the residue obtained was purified by RP-column to afford methyl 2-(1-(tetrahydro-2H-pyran-2-yl)-1H-pyrazol-3-yl)propanoate (60 mg, 28%) as a yellow solid. LCMS m/z=229.1 [M+H]⁺; ¹H NMR (400 MHz, CD₃OD) δ 7.72-7.68 (m, 1H), 6.24 (d, J=2.5 Hz, 1H), 5.33 (dd, J=10.1, 2.5 Hz, 1H), 4.02 (dp, J=11.6, 2.1 Hz, 1H), 3.85 (q, J=7.3 Hz, 1H), 3.67 (s, 3H), 2.15-1.92 (m, 4H), 1.80-1.55 (m, 4H), 1.47 (d, J=7.3 Hz, 3H)

### Step 6: 2-(1-(tetrahydro-2H-pyran-2-yl)-1H-pyrazol-3-yl)propanoic acid

To a solution of methyl 2-(1-(tetrahydro-2H-pyran-2-yl)-1H-pyrazol-3-yl)propanoate (40 mg, 0.16 mmol) in MeOH (1 mL) was added LiOH H₂O (8 mg, 0.2 mmol). The reaction was stirred at room temperature for 4 h then the solvent was removed under reduced pressure. The residue was diluted with water (1 mL) and acidified to pH˜5 with 1M HCl. The solvent was removed under vacuum to afford 2-(1-(tetrahydro-2H-pyran-2-yl)-1H-pyrazol-3-yl)propanoic acid (37 mg, quant.) as a yellow oil, which was used directly in the next step. LCMS m/z=224.9 [M+H]⁺.

### Synthesis of 2-(3-chloro-4-(tetrahydro-2H-pyran-4-yl)phenyl)acetohydrazide

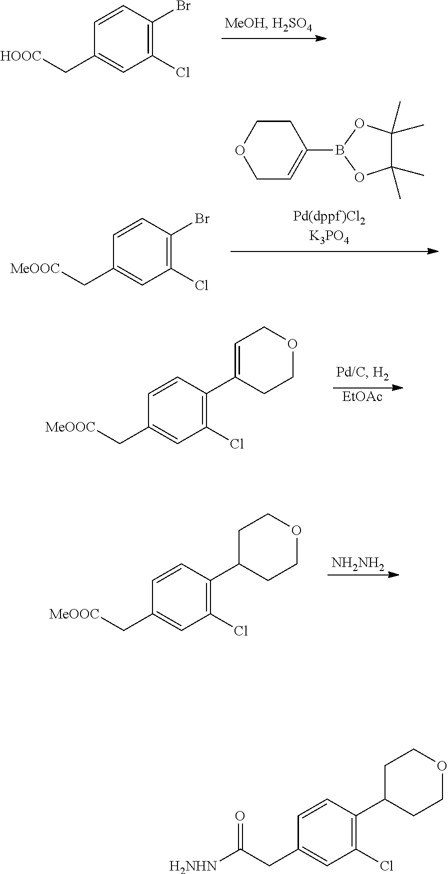

### Step 1: methyl 2-(4-bromo-3-chlorophenyl)acetate

To a solution of 2-(4-bromo-3-chlorophenyl)acetic acid (100 mg, 0.4 mmol, 1.0 eq) in MeOH (10 mL) was added H₂SO₄ (2 drop) and the reaction heated at reflux overnight. The mixture was diluted with water (20 mL) and extracted with EtOAc (50 mL×2). The combined organic layers were washed with brine, dried over Na₂SO₄, filtered and concentrated to afford the methyl 2-(4-bromo-3-chlorophenyl)acetate (90 mg, 87%) as a colorless oil.

### Step 2: methyl 2-(3-chloro-4-(3,6-dihydro-2H-pyran-4-yl)phenyl)acetate

To a solution of methyl 2-(4-bromo-3-chlorophenyl)acetate (200 mg, 0.8 mmol, 1.0 eq) in a mixture of 1,4-dioxane and water (5 mL/1 mL) under a N₂ atmosphere was added 2-(3,6-dihydro-2H-pyran-4-yl)-4,4,5,5-tetramethyl-1,3,2-dioxaborolane (191 mg, 0.9 mmol, 1.2 eq.), Pd(dppf)Cl₂ (28 mg, 0.04 mmol, 0.05 eq.) and K₃PO₄ (483 mg, 2.3 mmol, 3.0 eq.). The mixture was heated at 90° C. overnight then was diluted with water (50 mL) and extracted with EtOAc (100 mL×2). The combined organic layers were washed with brine, dried over Na₂SO₄, filtered and concentrated. The residue was purified by column chromatography on silica gel (eluent: Pet. Ether:EtOAc=10:1 to 5:1) to afford methyl 2-(3-chloro-4-(3,6-dihydro-2H-pyran-4-yl)phenyl)acetate (120 mg, 59%) as a yellow oil. ¹H NMR (400 MHz, CD₃OD) δ 7.32 (s, 1H), 7.18 (d, J=1.0 Hz, 2H), 5.74 (tt, J=2.9, 1.7 Hz, 1H), 4.27 (q, J=2.8 Hz, 2H), 3.90 (t, J=5.4 Hz, 2H), 3.69 (s, 3H), 3.64 (s, 2H), 2.43 (tdd, J=5.4, 4.4, 2.6 Hz, 2H)

### Step 3: methyl 2-(3-chloro-4-(tetrahydro-2H-pyran-4-yl)phenyl)acetate

To a solution of methyl 2-(3-chloro-4-(3,6-dihydro-2H-pyran-4-yl)phenyl)acetate (50 mg, 0.2 mmol, 1.0 eq.) in MeOH (5 mL) was added 5% Rh/C (5 mg). The mixture was stirred at room temperature for 5 h under a H₂ atmosphere then the catalyst was removed by filtration through Celite. The filtrate was concentrated to afford methyl 2-(3-chloro-4-(tetrahydro-2H-pyran-4-yl)phenyl)acetate (40 mg, 80%) as a yellow oil. ¹H NMR (400 MHz, CD₃OD) δ 7.34-7.27 (m, 1H), 7.22-7.15 (m, 1H), 4.09-4.00 (m, 1H), 3.65-3.52 (m, 2H), 1.82-1.70 (m, 2H), 1.37-1.20 (m, 1H).

### Step 4: 2-(3-chloro-4-(tetrahydro-2H-pyran-4-yl)phenyl)acetohydrazide

To a solution of methyl 2-(3-chloro-4-(tetrahydro-2H-pyran-4-yl)phenyl)acetate (400 mg, 0.15 mmol, 1.0 eq.) in EtOH (1 mL) was added 98% hydrazine hydrate (22 mg, 0.5 mmol, 3.0 eq.). The mixture was heated at 70° C. overnight the the solvent was removed under reduced pressure to afford 2-(3-chloro-4-(tetrahydro-2H-pyran-4-yl)phenyl)acetohydrazide (35 mg, 88%) as a yellow solid. LCMS m/z=269.2 [M+H]⁺.

### Synthesis of 2-(4-cyclopropylphenyl)-2,2-difluoroacetohydrazide

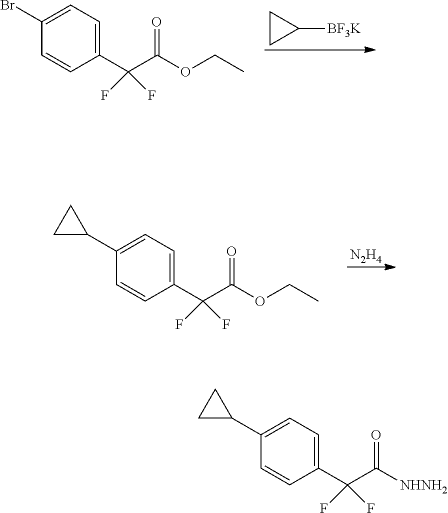

### Step 1: ethyl 2-(4-cyclopropylphenyl)-2,2-difluoroacetate

To a solution of ethyl 2-(4-bromophenyl)-2,2-difluoroacetate (50 mg, 0.18 mmol, 1.0 eq.) in a mixture of toluene and water (3 mL/1 mL) under N₂ atmosphere was added potassium cyclopropyltrifluoroborate (29 mg, 0.2 mmol, 1.1 eq.), tricyclohexyl phosphine (2 mg, 7 umol, 0.04 eq.), Pd(OAc)₂ (0.8 mg, 3.5 umol, 0.02 eq.) and K₂CO₃ (50 mg, 0.36 mmol, 2.0 eq.). The reaction was heated at 90° C. overnight then was diluted with water (10 mL) and extracted with EtOAc (25 mL×2). The combined organic layers were washed with brine, dried over Na₂SO₄, filtered and concentrated. The residue was purified by RP-column to afford the ethyl 2-(4-cyclopropylphenyl)-2,2-difluoroacetate (26 mg, 50%) as a white solid. ¹H NMR (400 MHz, CD₃OD) δ 7.44 (d, J=8.1 Hz, 2H), 7.17 (d, J=8.1 Hz, 2H), 4.27 (q, J=7.1 Hz, 2H), 1.95 (m, J=8.4, 5.1 Hz, 1H), 1.26 (t, J=7.1 Hz, 3H), 1.05-0.98 (m, 2H), 0.76-0.70 (m, 2H).

### Step 2: 2-(4-cyclopropylphenyl)-2,2-difluoroacetohydrazide

To a solution of ethyl 2-(4-cyclopropylphenyl)-2,2-difluoroacetate (25 mg, 0.1 mmol, 1.0 eq.) in MeOH (1 mL) was added 98% hydrazine hydrate (5 drops). The resulting mixture was stirred at room temperature for 3 h then was diluted with water (10 mL) and extracted with DCM/MeOH (10/1, 25 mL×2). The combined organic layers were washed with brine, dried over Na₂SO₄, filtered and concentrated to afford 2-(4-cyclopropylphenyl)-2,2-difluoroacetohydrazide (10 mg, 43%) as a white solid. LCMS m/z=227.1 [M+H]⁺.

### Synthesis of 2-methyl-2-(1-(tetrahydro-2H-pyran-2-yl)-1H-pyrazol-3-yl)propanoic acid

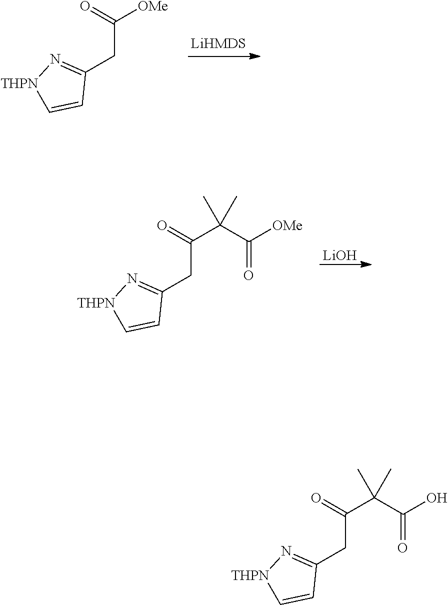

### Step 1: methyl 2-methyl-2-(1-(tetrahydro-2H-pyran-2-yl)-1H-pyrazol-3-yl)propanoate

To a solution of methyl 2-(1-(tetrahydro-2H-pyran-2-yl)-1H-pyrazol-3-yl)acetate (200 mg, 0.9 mmol) in anhydrous THE (2 mL) was added LiHMDS (1 M in THF, 1.8 mL, 1.78 mmol). The mixture was stirred at 0° C. for 30 min then CH₃I (319 mg, 2.2 mmol, 2.5 eq.) was added. The reaction was stirred at room temperature for 2 h then was concentrated under reduced pressure. The residue obtained was purified by RP-column to afford methyl 2-methyl-2-(1-(tetrahydro-2H-pyran-2-yl)-1H-pyrazol-3-yl)propanoate (120 mg, 53%) as a yellow solid. LCMS m/z=253.1 [M+H]⁺.

### Step 2: 2-methyl-2-(1-(tetrahydro-2H-pyran-2-yl)-1H-pyrazol-3-yl)propanoic acid

To a solution of methyl 2-methyl-2-(1-(tetrahydro-2H-pyran-2-yl)-1H-pyrazol-3-yl)propanoate (80 mg, 0.3 mmol) in MeOH (1 mL) was added LiOH H₂O (16 mg, 0.4 mmol). The reaction was stirred at room temperature for 4 h then the solvent was removed under reduced pressure. The residue was diluted with water (1 mL) and acidified to pH˜5 with 1M HCl. The solvent was removed under vacuum to afford 2-methyl-2-(1-(tetrahydro-2H-pyran-2-yl)-1H-pyrazol-3-yl)propanoic acid (75 mg, quant.) as a yellow oil, which was used directly in the next step. LCMS m/z=225.4 [M+H]⁺.

### Synthesis of methyl 2-(2H-indazol-2-yl)propanoate (A-0910-1) and methyl 2-(1H-indazol-1-yl)propanoate

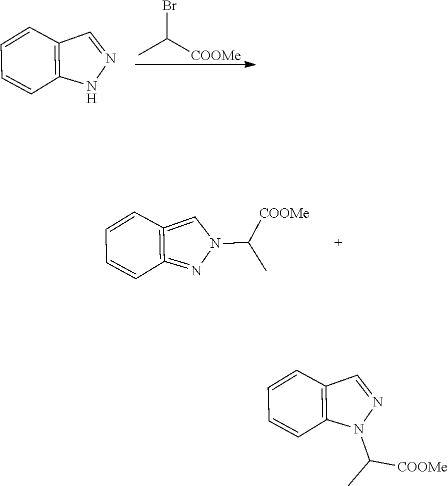

### Step 1: methyl 2-(2H-indazol-2-yl)propanoate (A-0910-1) and methyl 2-(1H-indazol-1-yl)propanoate

To a solution of indazole (1.0 g, 8.46 mmol) in DMF (3.0 mL) at 0° C. was added NaH (204 mg, 8.46 mg). The mixture was stirred for 30 min then methyl 2-bromopropanoate (1.69 g, 9.32 mmol) was added. The reaction was allowed to warm to room temperature and was stirred overnight. The reaction was quenched with water (50 mL) and extracted with EtOAc (100 mL×2). The combined organic layers were washed with brine, dried over Na₂SO₄, filtered and concentrated. The residue obtained was purified by column chromatography on silica gel (eluent: Pet. ether/EtOAc=5/1 to 2/1) to afford methyl 2-(1H-indazol-1-yl)propanoate (861 mg, 46%) as a colorless oil as the first eluting regioisomer. LCMS m/z=256.2 [M+H]⁺; ¹H NMR (400 MHz, Methanol-d₄) δ 8.04 (s, 1H), 7.75 (d, J=8.1 Hz, 1H), 7.53 (d, J=8.4 Hz, 1H), 7.40 (t, J=8.0 Hz, 1H), 7.17 (t, J=7.5 Hz, 1H), 5.55 (q, J=7.2 Hz, 1H), 4.13 (q, J=7.1 Hz, 2H), 1.85 (d, J=7.2 Hz, 3H), 1.14 (t, J=7.2 Hz, 3H). Further elution provided methyl 2-(2H-indazol-2-yl)propanoate (327 mg, 17%) as a colorless oil. LCMS m/z=256.2 [M+H]⁺; ¹H NMR (400 MHz, Methanol-d₄) δ 8.30 (s, 1H), 7.69 (d, J=8.5 Hz, 1H), 7.59 (d, J=8.8 Hz, 1H), 7.31-7.25 (m, 1H), 7.10-7.03 (m, 1H), 5.45 (q, J=7.3 Hz, 1H), 4.24-4.08 (m, 2H), 1.88 (d, J=7.3 Hz, 3H), 1.19 (t, J=7.1 Hz, 3H).

### Synthesis of 2-(2H-indazol-2-yl)propanoic acid

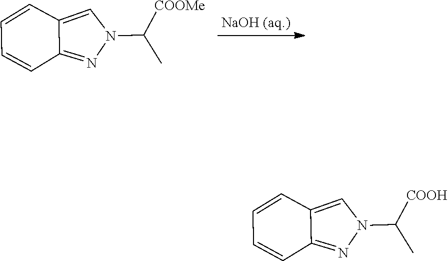

### Step 1: 2-(2H-indazol-2-yl)propanoic acid

To a solution of methyl 2-(2H-indazol-2-yl)propanoate (300 mg, 1.37 mmol) in THE (2.0 mL) was added 1 M NaOH (4.0 mL). The reaction was stirred at room temperature for 2 h then was diluted with water (20 mL) and extracted with EtOAc (50 mL). The aqueous layer was collected and acidified to pH˜2 with 1M HCl then was extracted with EtOAc (100 mL×3). The combined organic layers were washed with brine, dried over Na₂SO₄, filtered and concentrated to afford 2-(2H-indazol-2-yl)propanoic acid (247 mg, 95%) as a white solid. LCMS m/z=389.2 [M+H]⁺.

### Synthesis of 2-(1H-indazol-1-yl)propanoic acid

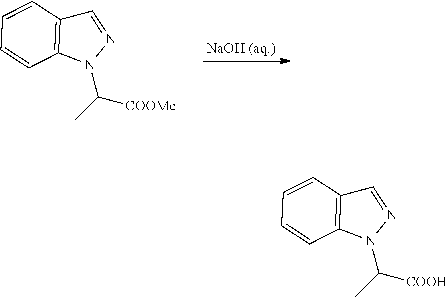

### Step 1: 2-(1H-indazol-1-yl)propanoic acid

To a solution of methyl 2-(1H-indazol-1-yl)propanoate (400 mg, 1.83 mmol) and in THF (2.0 mL) was 1 M NaOH (6.0 mL). The reaction was stirred at room temperature for 2 h then was diluted with water (20 mL) and extracted with EtOAc (50 mL). The aqueous layer was collected and acidified to pH˜2 with 1M HCl then extracted with EtOAc (100 mL×3). The combined organic layers were washed with brine, dried over Na₂SO₄, filtered and concentrated to afford 2-(1H-indazol-1-yl)propanoic acid (324 mg, 93%) as a white solid. LCMS m/z=389.2 [M+H]⁺.

### Synthesis of 2-(6-(tetrahydro-2H-pyran-4-yl)pyridin-2-yl)propanehydrazide

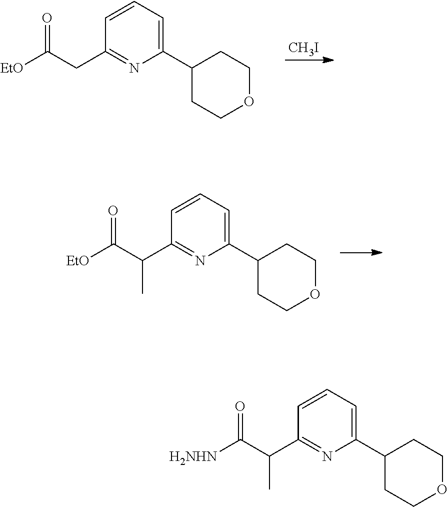

### Step 1: ethyl 2-(6-(tetrahydro-2H-pyran-4-yl)pyridin-2-yl)propanoate

To a solution of ethyl 2-(6-(tetrahydro-2H-pyran-4-yl)pyridin-2-yl)acetate (200 mg, 0.8 mmol) in THF (4 mL) at 0° C. was added LiHMDS (0.96 mL, 1 M in THF). After stirring for 20 min, CH₃I (150 mg, 1.04 mmol) was added and the reaction stirred for 3 hours. The mixture was diluted with water (50 mL) and extracted with EtOAc (100 mL×2). The combined organic layers were washed with brine, dried over Na₂SO₄, filtered and concentrated. The residue obtained was purified by RP-column to affort ethyl 2-(6-(tetrahydro-2H-pyran-4-yl)pyridin-2-yl)propanoate (56 mg, 27%) as a colorless oil. LCMS m/z=264.1 [M+H]⁺.

### Step 2: 2-(6-(tetrahydro-2H-pyran-4-yl)pyridin-2-yl)propanehydrazide

To a solution of ethyl 2-(6-(tetrahydro-2H-pyran-4-yl)pyridin-2-yl)propanoate (50 mg, 0.19 mmol) in MeOH (0.5 mL) was added 98% hydrazine hydrate (4 drops). The resulting mixture was stirred at room temperature for 2 h then was concentrated under vacuum to afford 2-(6-(tetrahydro-2H-pyran-4-yl)pyridin-2-yl)propanehydrazide (54 mg, quant.) as a yellow oil. ¹H NMR (400 MHz, DMSO-d₆) δ 9.17 (s, 1H), 7.65 (t, J=7.7 Hz, 1H), 7.21 (d, J=7.7 Hz, 1H), 7.12 (d, J=7.7 Hz, 1H), 3.94 (dt, J=11.1, 3.3 Hz, 2H), 3.67 (q, J=7.1 Hz, 1H), 3.44 (ddd, J=11.3, 8.5, 5.7 Hz, 4H), 2.88 (ddd, J=15.6, 9.2, 6.8 Hz, 1H), 1.74 (h, J=4.1 Hz, 4H), 1.38 (d, J=7.1 Hz, 3H).

### Synthesis of 2-(4-chlorophenyl)acetohydrazide

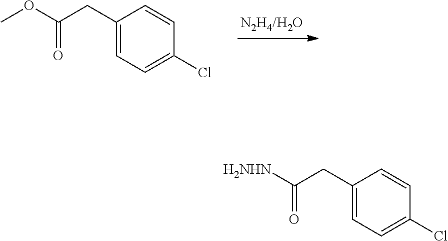

### Step 1: 2-(4-chlorophenyl)acetohydrazide

To a solution of methyl 2-(4-chlorophenyl)acetate (500 mg, 2.7 mmol) in MeOH (1.0 mL) was added 98% hydrazine hydrate (0.5 mL) and the reaction heated at 70° C. for 30 min. The solid precipitate that formed was collected by filtration and washed with cold MeOH to give 2-(4-chlorophenyl)acetohydrazide (363 mg, 73% yield) as a white solid. LCMS m/z=185.1 [M+H]⁺.

### Synthesis of 2-(4-cyanophenyl)-2,2-difluoroacetohydrazide

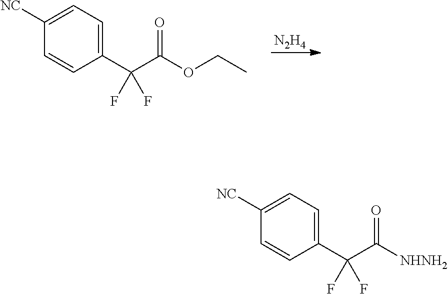

### Step 1: 2-(4-cyanophenyl)-2,2-difluoroacetohydrazide

To a solution of ethyl 2-(4-cyanophenyl)-2,2-difluoroacetate (500 mg, 2.22 mmol) in MeOH (3 mL) was added 98% hydrazine hydrate (2 mL) and the reaction stirred at room temperature for 20 min. The mixture was concentrated under vacuum and the residue obtained triturated with cold MeOH to afford crude 2-(4-cyanophenyl)-2,2-difluoroacetohydrazide (421 mg, 90%) as a yellow solid. LCMS m/z=212.1 [M+H]⁺.

### Synthesis of 2-(benzo[d]oxazol-2-yl)acetohydrazide

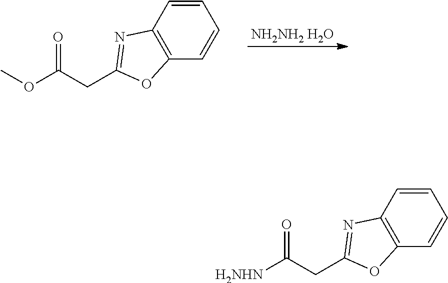

### Step 1: 2-(benzo[d]oxazol-2-yl)acetohydrazide

To a solution of methyl 2-(benzo[d]oxazol-2-yl)acetate (100 mg, 0.521 mmol) in MeOH (2.0 mL) was added 98% hydrazine hydrate (80.8 mg, 1.612 mmol) and the reaction heated at 70° C. for 2 h. The mixture was concentrated under vacuum and the residue obtained triturated with cold MeOH to afford 2,2-difluoro-2-(4-methoxyphenyl)acetohydrazide (90 mg, 90%) as a white solid. ¹H NMR (400 MHz, DMSO-d6) δ 9.43 (s, 1H), 7.72-7.65 (m, 2H), 7.39-7.33 (m, 2H), 4.34 (d, J=4.0 Hz, 2H), 3.83 (s, 2H).

### Synthesis of 2-(4-bromophenyl)-2,2-difluoroacetohydrazide

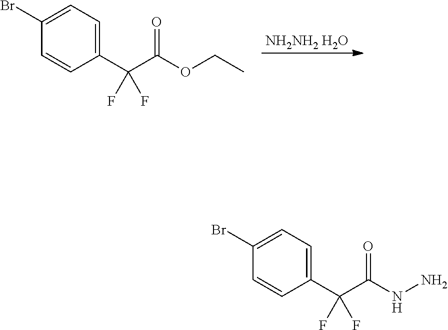

### Step 1: 2-(4-bromophenyl)-2,2-difluoroacetohydrazide

To a solution of ethyl 2,2-difluoro-2-(4-methoxyphenyl)etate (150 mg, 0.537 mmol) in MeOH (2.0 mL) was added 98% hydrazine hydrate (80.8 mg, 1.612 mmol) and the reaction stirred at room temperature for 30 min. The mixture was concentrated under vacuum and the obtained triturated with cold MeOH to afford 2,2-difluoro-2-(4-methoxyphenyl)acetohydrazide (120 mg, 86%) as a white solid. LCMS m/z=264.7 [M+H]⁺.

### Synthesis of 2,2-difluoro-2-(4-methoxyphenyl)acetohydrazide

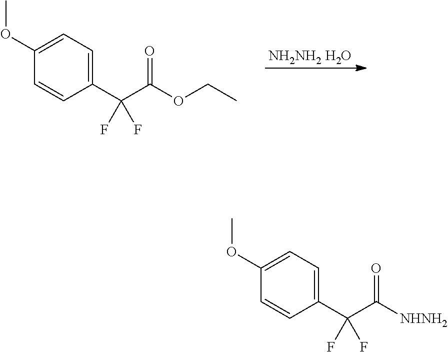 [SMILES: CCOC(=O)C(F)(F)c1ccc(OC)cc1 ; "NH2NH2 H2O" ; COc1ccc(C(F)(F)C(=O)NN)cc1]

### Step 1: 2,2-difluoro-2-(4-methoxyphenyl)acetohydrazide

To a solution of ethyl 2,2-difluoro-2-(4-methoxyphenyl)acetate (200 mg, 0.434 mmol) in MeOH (2.0 mL) was added 98% hydrazine hydrate (65.3 mg, 1.303 mmol) and the reaction stirred at room temperature for 30 min. The mixture was concentrated under vacuum and the residue obtained triturated with cold MeOH to afford 2,2-difluoro-2-(4-methoxyphenyl)acetohydrazide (160 mg, 85%) as a white solid. LCMS m/z=216.9 [M+H]⁺; ¹H NMR (400 MHz, CD₃OD) δ 7.52 (d, J=8.9 Hz, 2H), 7.00 (d, J=8.8 Hz, 2H), 3.82 (s, 3H).

### Synthesis of 2-(3-cyclopentyl-1H-pyrazol-1-yl)propanehydrazide

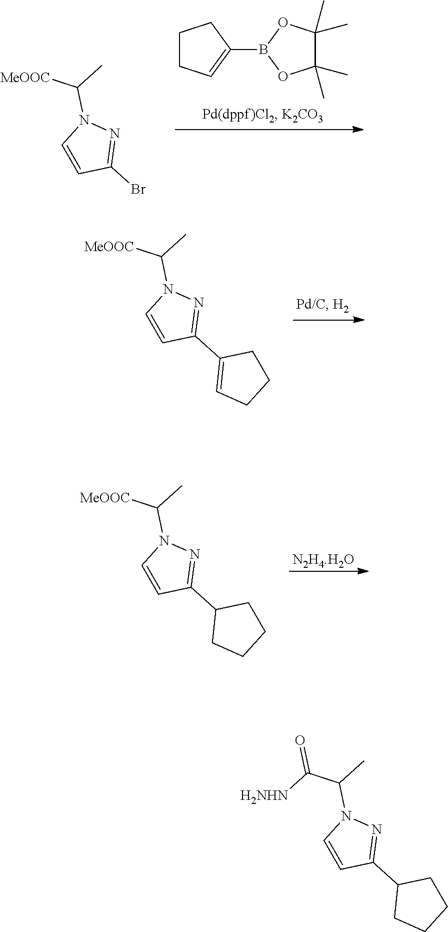

### Step 1: methyl 2-(3-(cyclopent-1-en-1-yl)-1H-pyrazol-1-yl)propanoate

To a solution of methyl 2-(3-bromo-1H-pyrazol-1-yl)propanoate (1 g, 4.3 mmol) in a mixture of dioxane and water (10 mL/0.5 mL) under a N₂ atmosphere was added 2-(cyclopent-1-en-1-yl)-4,4,5,5-tetramethyl-1,3,2-dioxaborolane (1.25 g, 6.5 mmol), Pd(dppf)Cl₂ (629 mg, 0.86 mmol) and K₂CO₃ (1.78 g, 12.9 mmol). The reaction was heated at 110° C. for 8 h then was diluted with water (50 mL) and extracted with EtOAc (100 mL×2). The combined organic layers were washed with brine, dried over Na₂SO₄, filtered and concentrated. The residue was purified by column chromatography on silica gel (eluent: Pet. ether/EtOAc=5/1) to afford methyl 2-(3-(cyclopent-1-en-1-yl)-1H-pyrazol-1-yl)propanoate (793 mg, 84%) as a yellow oil. LCMS m/z=221.2 [M+H]⁺; ¹H NMR (400 MHz, Chloroform-d) δ 7.48 (d, J=2.4 Hz, 1H), 6.38 (d, J=2.4 Hz, 1H), 6.18-6.14 (m, 1H), 5.14 (q, J=7.4 Hz, 1H), 3.73 (s, 3H), 2.75-2.68 (m, 2H), 2.52-2.45 (m, 2H), 2.00-1.91 (m, 2H), 1.77 (d, J=7.4 Hz, 3H).

### Step 2: methyl 2-(3-cyclopentyl-1H-pyrazol-1-yl)propanoate

To a solution of methyl 2-(3-(cyclopent-1-en-1-yl)-1H-pyrazol-1-yl)propanoate (300 mg, 1.36 mmol) in MeOH (3 mL) was added 10% Pd/C (100 mg). The reaction was stirred under H₂ overnight then the catalyst was removed by filtration through celite. The filtrate was concentrated to afford methyl 2-(3-cyclopentyl-1H-pyrazol-1-yl)propanoate (304 mg, quant.) as a black oil. LCMS m/z=223.2 [M+H]⁺; ¹H NMR (400 MHz, Chloroform-d) δ 7.43 (d, J=2.4 Hz, 1H), 6.12 (d, J=2.4 Hz, 1H), 5.06 (q, J=7.4 Hz, 1H), 3.72 (s, 3H), 3.15-3.02 (m, 1H), 2.04 (s, 2H), 1.75 (d, J=7.2 Hz, 3H), 1.73-1.56 (m, 6H).

### Step 3: 2-(3-cyclopentyl-1H-pyrazol-1-yl)propanehydrazide

To a solution of methyl 2-(3-cyclopentyl-1H-pyrazol-1-yl)propanoate (130 mg, 0.58 mmol) in MeOH (1 mL) was added 98% hydrazine hydrate (1 mL) and the reaction stirred at room temperature overnight. The mixture was concentrated under vacuum to afford 2-(3-cyclopentyl-1H-pyrazol-1-yl)propanehydrazide (100 mg, 85%) as a yellow oil. LCMS m/z=232.2 [M+H]⁺.

### Synthesis of 3-(4,4-difluorocyclohexyl)propanoic acid

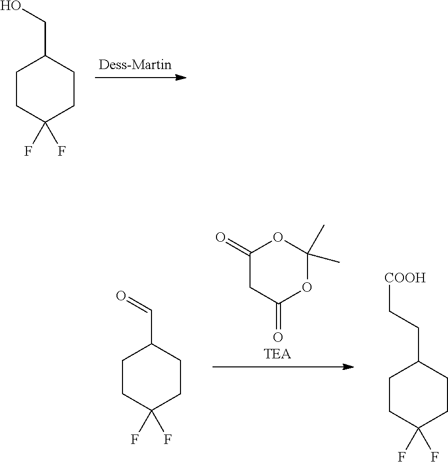

### Step 1: 4,4-difluorocyclohexane-1-carbaldehyde

To a solution of (4,4-difluorocyclohexyl)methanol (0.5 g, 3.33 mmol) in DCM (10 mL) at 0° C. was added dess-martin reagent (1.55 g, 3.66 mmol). The reaction mixture was stirred at room temperature for 14 h then was quenched with aq. Na₂S₂O₄ and extracted with DCM (50 mL×2). The combined organic layers were washed with water and brine and dried over Na₂SO₄, filtered and concentrated to afford 4,4-difluorocyclohexane-1-carbaldehyde (200 mg, 40%) as a colorless oil. ¹H NMR (400 MHz, Chloroform-d) δ 9.67 (s, 1H), 2.40-2.29 (m, 1H), 2.13-1.94 (m, 4H), 1.92-1.72 (m, 4H).

### Step 2: 3-(4,4-difluorocyclohexyl)propanoic acid

A mixture of TEA (2.68 g, 26.5 mmol) and formic acid (3.0 g, 65 mmol) was stirred at 0° C. for 10 min. 4,4-difluorocyclohexane-1-carbaldehyde (370 mg, 2.5 mmol) and 2,2-dimethyl-1,3-dioxane-4,6-dione (360 mg, 2.5 mmol) were added and th reaction heated at 110° C. for 5 h. The reaction was made basic (pH˜10) with aq. NaOH and extracted with MTBE (30 mL×2). The aqueous phase was collected and acidified to pH˜1 with 2 M HCl then extracted with DCM (50 mL×2). The combined organic layers were washed with brine, dried over Na₂SO₄, filtered and concentrated to afford 3-(4,4-difluorocyclohexyl)propanoic acid (200 mg, 42%) as a yellow solid. ¹H NMR (400 MHz, Chloroform-d) δ 2.39 (t, J=7.6 Hz, 2H), 2.17-1.99 (m, 2H), 1.84-1.56 (m, 6H), 1.47-1.19 (m, 3H).

### Synthesis of 2-(3,5-dimethyl-1H-pyrazol-1-yl)propanoic acid

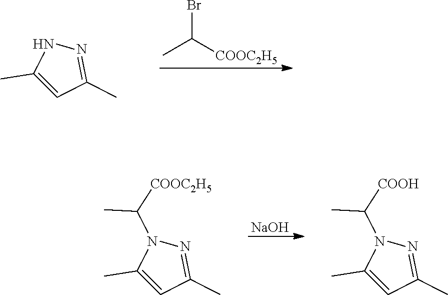

### Step 1: ethyl 2-(3,5-dimethyl-1H-pyrazol-1-yl)propanoate

To a solution of 3,5-dimethyl-1H-pyrazole (500 mg, 5.20 mmol) in THE (10.0 mL) at 0° C. was added NaH (250 mg, 6.24 mmol). The reaction mixture was stirred at 0° C. for 30 min then ethyl 2-bromopropanoate (1.22 g, 6.76 mmol) was added. The reaction was allowed to warm to room temperature and stirred for another 2 h then was diluted with water (30 mL), extracted with DCM (60 mL×2). The combined organic layers were washed with brine, dried over Na₂SO₄, filtered and concentrated to afford ethyl 2-(3,5-dimethyl-1H-pyrazol-1-yl)propanoate (600 mg, 59%) as a colorless oil. LCMS m/z=196.9 [M+H]⁺; ¹H NMR (400 MHz, DMSO-d6) δ 5.80 (s, 1H), 5.08 (q, J=7.1 Hz, 1H), 4.07 (ddp, J=13.7, 7.1, 3.6 Hz, 2H), 2.17 (s, 3H), 2.07 (s, 3H), 1.58 (d, J=7.0 Hz, 3H), 1.13 (t, J=7.0 Hz, 3H).

### Step 2: 2-(3,5-dimethyl-1H-pyrazol-1-yl)propanoic acid

To a solution of ethyl 2-(3,5-dimethyl-1H-pyrazol-1-yl)propanoate (500 mg, 2.55 mmol) in a mixture of MeOH and water (0.8 mL/0.2 mL) was added NaOH (306 mg, 7.65 mmol). The reaction was stirred at room temperature for 4 h then was diluted with water (20 mL) and extracted with EtOAc (50 mL). The aqueous layer was collected and acidified to pH˜2 with 1M HCl then extracted with EtOAc (100 mL×3). The combined organic layers were washed with brine, dried over Na₂SO₄, filtered and concentrated. The residue was purified by reverse phase column (65% acetonitrile in water) to afford 2-(3,5-dimethyl-1H-pyrazol-1-yl)propanoic acid (300 mg, 70%) as a yellow oil. LCMS: m/z=168.9 [M+H]⁺.

### Synthesis of (8-(5-(3-chloro-4-(trifluoromethyl)benzyl)oxazol-2-yl)-2-((S)-2,2-dimethylcyclopropane-1-carbonyl)-2,6-diazaspiro[3.4]octan-6-yl)(thiazol-5-yl)methanone (I-201)

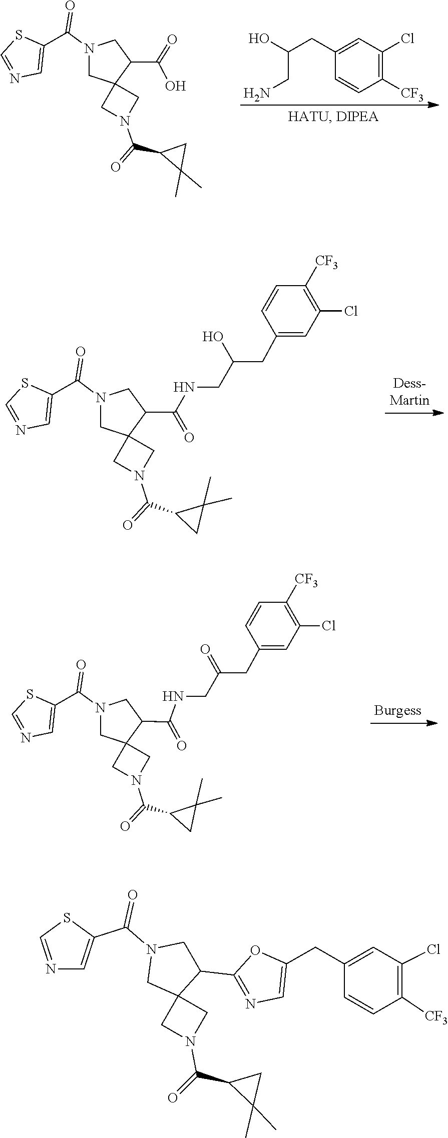 [SMILES: CC1(C)C[C@@H]1C(=O)N1CC2(CN(C(=O)c3cncs3)CC2C(=O)O)C1 ; NCC(O)Cc1ccc(C(F)(F)F)c(Cl)c1 ; CC1(C)C[C@@H]1C(=O)N1CC2(CN(C(=O)c3cncs3)CC2C(=O)NCC(O)Cc2ccc(C(F)(F)F)c(Cl)c2)C1 ; CC1(C)C[C@@H]1C(=O)N1CC2(CN(C(=O)c3cncs3)CC2C(=O)NCC(=O)Cc2ccc(C(F)(F)F)c(Cl)c2)C1 ; CC1(C)C[C@@H]1C(=O)N1CC2(CN(C(=O)c3cncs3)CC2c2ncc(Cc3ccc(C(F)(F)F)c(Cl)c3)o2)C1]

(8-(5-(3-chloro-4-(trifluoromethyl)benzyl)oxazol-2-yl)-2-((S)-2,2-dimethylcyclopropane-1-carbonyl)-2,6-diazaspiro[3.4]octan-6-yl)(thiazol-5-yl)methanone was synthesized from 2-((S)-2,2-dimethylcyclopropane-1-carbonyl)-6-(thiazole-5-carbonyl)-2,6-diazaspiro[3.4]octane-8-carboxylic acid according to the procedures outlined for I-48 using the appropriate commercially available reagents and/or intermediates described elsewhere. LCMS m/z=579.0 [M+H]⁺; ¹H NMR (400 MHz, CD₃OD) δ 9.16 (s, 1H), 8.38 (s, 1H), 7.72 (s, 1H), 7.54 (s, 1H), 7.34 (s, 1H), 6.96 (s, 1H), 4.48-3.82 (m, 11H), 1.41 (s, 1H), 1.13-1.03 (m, 5H), 1.02-0.95 (m, 2H), 0.73 (s, 1H).

### Synthesis of (8-(5-(difluoro(4-(3,3,3-trifluoroprop-1-en-2-yl)phenyl)methyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone (I-198)

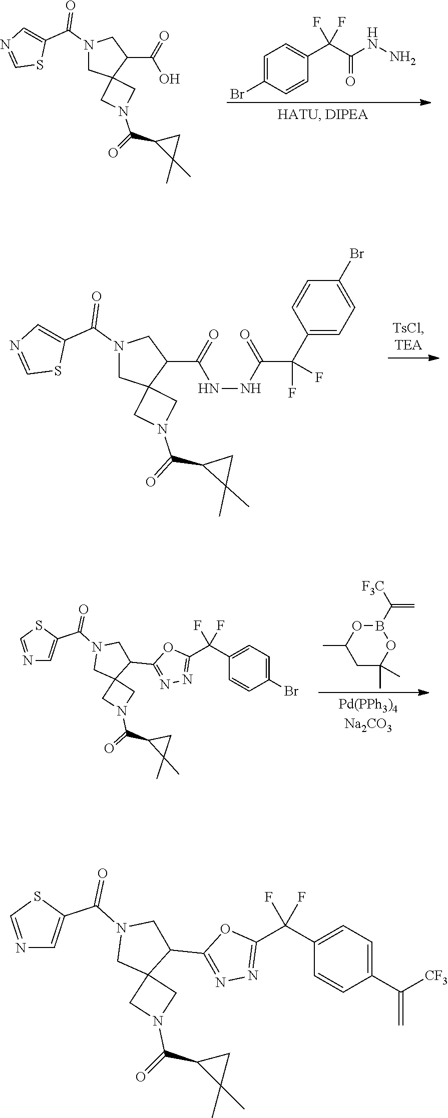 [SMILES: CC1(C)C[C@@H]1C(=O)N1CC2(CN(C(=O)c3cncs3)CC2C(=O)O)C1 ; NNC(=O)C(F)(F)c1ccc(Br)cc1 ; CC1(C)C[C@@H]1C(=O)N1CC2(CN(C(=O)c3cncs3)CC2C(=O)NNC(=O)C(F)(F)c2ccc(Br)cc2)C1 ; CC1(C)C[C@@H]1C(=O)N1CC2(CN(C(=O)c3cncs3)CC2c2nnc(C(F)(F)c3ccc(Br)cc3)o2)C1 ; C=C(B1OC(C)CC(C)(C)O1)C(F)(F)F ; C=C(c1ccc(C(F)(F)c2nnc(C3CN(C(=O)c4cncs4)CC34CN(C(=O)[C@H]3CC3(C)C)C4)o2)cc1)C(F)(F)F]

### Step 1: N′-(2-(4-bromophenyl)-2,2-difluoroacetyl)-2-((S)-2,2-dimethylcyclopropane-1-carbonyl)-6-(thiazole-5-carbonyl)-2,6-diazaspiro[3.4]octane-8-carbohydrazide

To a solution of 2-((S)-2,2-dimethylcyclopropane-1-carbonyl)-6-(thiazole-5-carbonyl)-2,6-diazaspiro[3.4]octane-8-carboxylic acid (450 mg, 1.24 mmol) in DCM (5 mL) was added HATU (706 mg, 1.86 mmol) and DIPEA (880 mg, 3.71 mmol). The mixture was stirred at room temperature for 30 min then 2-(4-bromophenyl)-2,2-difluoroacetohydrazide (176 mg, 1.86 mmol) was added and the reaction stirred overnight. The reaction was quenched with water (10 mL) and extracted with DCM (20 mL×3). The combined organic layers was washed with brine, dried over Na₂SO₄, filtered and concentrated. The residue was purified by column chromatography on silica gel (eluent: DCM/MeOH=30/1) to afford N′-(2-(4-bromophenyl)-2,2-difluoroacetyl)-2-((S)-2,2-dimethylcyclopropane-1-carbonyl)-6-(thiazole-5-carbonyl)-2,6-diazaspiro[3.4]octane-8-carbohydrazide (280 mg, 37%) as a white solid. LCMS m/z=610.1 [M+H]⁺; ¹H NMR (400 MHz, CD₃OD)¹H NMR (400 MHz, DMSO-d₆) δ 11.09 (s, 1H), 10.43 (d, J=11.4 Hz, 1H), 9.25 (s, 1H), 8.38 (d, J=19.2 Hz, 1H), 7.78 (d, J=8.2 Hz, 2H), 7.58 (d, J=8.1 Hz, 2H), 4.22-3.72 (m, 8H), 3.29-3.20 (m, 1H), 1.41-1.27 (m, 1H), 1.13-1.03 (m, 6H), 0.89-0.82 (m, 1H), 0.76-0.63 (m, 1H).

### Step 2: (8-(5-((4-bromophenyl)difluoromethyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone

To a solution of N′-(2-(4-bromophenyl)-2,2-difluoroacetyl)-2-((S)-2,2-dimethylcyclopropane-1-carbonyl)-6-(thiazole-5-carbonyl)-2,6-diazaspiro[3.4]octane-8-carbohydrazide (200 mg, 0.33 mmol) in DCM (4 mL) was added TEA (100 mg, 0.99 mmol) and TsCl (189 mg, 0.99 mmol). The reaction mixture was stirred at room temperature overnight then was diluted with water (30 mL) and extracted with DCM (50 mL×3). The combined organic layers were washed with brine, dried over Na₂SO₄, filtered and concentrated. The residue obtained was purified by column chromatography on silica gel (eluent: DCM/MeOH=60/1) to afford (8-(5-((4-bromophenyl)difluoromethyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone (130 mg, 66%) as a white solid. LCMS m/z=592.1 [M+H]⁺; ¹H NMR (400 MHz, CD₃OD) δ 9.17 (s, 1H), 8.38 (d, J=6.2 Hz, 1H), 7.73 (d, J=8.1 Hz, 2H), 7.65-7.53 (m, 2H), 4.50-3.98 (m, 9H), 1.47-1.30 (m, 2H), 1.23-1.06 (m, 6H), 0.83-0.71 (m, 1H).

### Step 3: (8-(5-(difluoro(4-(3,3,3-trifluoroprop-1-en-2-yl)phenyl)methyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone (I-198)

To a solution of (8-(5-((4-bromophenyl)difluoromethyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone (110 mg, 0.19 mmol), 4,4,6-trimethyl-2-(3,3,3-trifluoroprop-1-en-2-yl)-1,3,2-dioxaborinane (64 mg, 0.29 mmol) and Na₂CO₃ (60 mg, 0.57 mmol) in a mixture of dioxane (4 mL) and water (1 mL) was added Pd(PPh₃)₄ (23 mg, 0.02 mmol). The reaction mixture was heated at 110° C. for 2 h then was diluted with water (10 mL) and extracted with EtOAc (20 mL×3). The combined organic layers were washed with brine, dried over Na₂SO₄, filtered and concentrated. The crude product was purified by prep-TLC (eluent: DCM:MeOH=15:1) to afford (8-(5-(difluoro(4-(3,3,3-trifluoroprop-1-en-2-yl)phenyl)methyl)-1,3,4-oxadiazol-2-yl)-2-((S)-2,2-dimethylcyclopropane-1-carbonyl)-2,6-diazaspiro[3.4]octan-6-yl)(thiazol-5-yl)methanone (37 mg, 32%) as a white solid. LCMS m/z=608.2 [M+H]⁺; ¹H NMR (400 MHz, CD₃OD) δ 9.16 (s, 1H), 8.38 (d, J=5.9 Hz, 1H), 7.83-7.60 (m, 4H), 6.09 (d, J=33.0 Hz, 2H), 4.58-3.92 (m, 9H), 1.33-0.97 (m, 8H), 0.81-0.71 (m, 1H).

Table 19: The compounds listed in Table 19 were synthesized from (8-(5-((3,4-dichlorophenyl)difluoromethyl)-1,3,4-oxadiazol-2-yl)-2,6-diazaspiro[3.4]octan-2-yl)((S)-2,2-dimethylcyclopropyl)methanone according to the procedures outlined for I-43 using the appropriate commercially available reagents and/or intermediates described elsewhere.

TABLE 19

| Example | | | |
|---|---|---|---|
| Number | Compound | ¹HNMR | LCMS |
| I-200 | 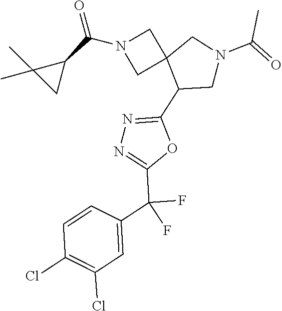 [SMILES: CC(=O)N1CC(c2nnc(C(F)(F)c3ccc(Cl)c(Cl)c3)o2)C2(C1)CN(C(=O)[C@H]1CC1(C)C)C2] | ¹H NMR (400 MHz, Methanol-d₄) δ 7.92- 7.85 (m, 1H), 7.78-7.72 (m, 1H), 7.66-7.58 (m, 1H), 4.71-3.74 (m, 10H), 2.13-2.02 (m, 3H), 1.46-1.23 (m, 1H), 1.20-0.96 (m, 6H), 0.83- 0.70 (m, 1H). | m/z = 513.1 [M + H]⁺ |
| I-185 | 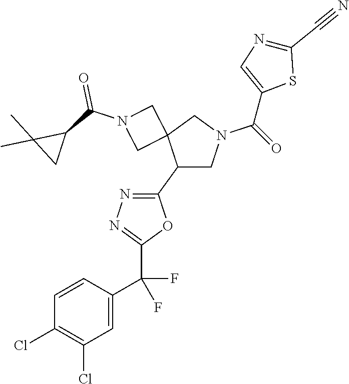 [SMILES: CC1(C)C[C@@H]1C(=O)N1CC2(CN(C(=O)c3cnc(C#N)s3)CC2c2nnc(C(F)(F)c3ccc(Cl)c(Cl)c3)o2)C1] | ¹H NMR (400 MHz, DMSO-d₆) δ 8.61 (dd, J = 17.6, 2.8 Hz, 1H), 8.01 (d, J = 11.4 Hz, 1H), 7.88 (dd, J = 8.4, 5.6 Hz, 1H), 7.71 (t, J = 8.4 Hz, 1H), 4.36-4.11 (m, 5H), 4.02- 3.83 (m, 4H), 1.30 (m, 1H), 1.13-1.09 (m, 2H), 1.07-1.03 (m, 2H), 0.99 (d, J = 6.2 Hz, 1H), 0.94 (s, 1H), 0.85 (s, 1H), 0.68 (s, 1H). | m/z = 607.2 [M + H]⁺ |
| I-199 | 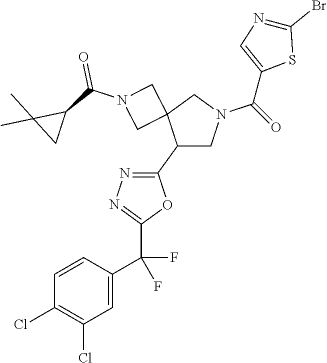 [SMILES: CC1(C)C[C@@H]1C(=O)N1CC2(CN(C(=O)c3cnc(Br)s3)CC2c2nnc(C(F)(F)c3ccc(Cl)c(Cl)c3)o2)C1] | ¹H NMR (400 MHz, DMSO-d₆) δ 8.15 (m, 1H), 8.01 (d, J = 5.6 Hz, 1H), 7.88 (t, J = 6.6 Hz, 1H), 7.74-7.68 (m, 1H), 4.34-4.08 (m, 5H), 4.02- 3.82 (m, 4H), 1.30 (m, 1H), 1.10 (d, J = 8.4 Hz, 2H), 1.05 (d, J = 3.6 Hz, 2H), 0.99 (d, J = 6.2 Hz, 1H), 0.94 (s, 1H), 0.85 (t, J = 4.4 Hz, 1H), 0.72- 0.64 (m, 1H). | m/z = 660.05 662.05 [M + H]⁺ |

### Synthesis of (8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)((S)-2,2-dimethylcyclopropyl)methanone (I-192)

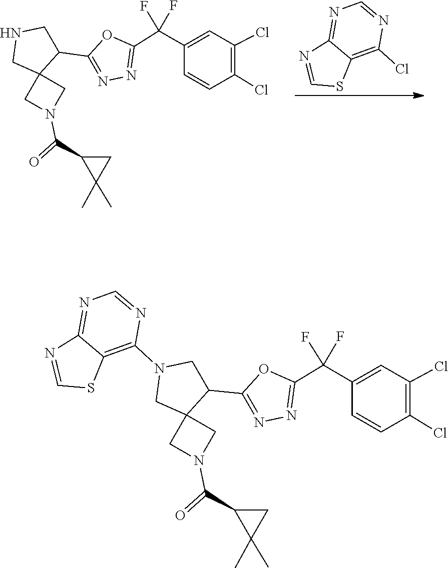 [SMILES: CC1(C)C[C@@H]1C(=O)N1CC2(CNCC2c2nnc(C(F)(F)c3ccc(Cl)c(Cl)c3)o2)C1 ; Clc1ncnc2ncsc12 ; CC1(C)C[C@@H]1C(=O)N1CC2(C1)CN(c1ncnc3ncsc13)CC2c1nnc(C(F)(F)c2ccc(Cl)c(Cl)c2)o1]

### Step 1: (8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)((S)-2,2-dimethylcyclopropyl)methanone

To a solution of (8-(5-((3,4-dichlorophenyl)difluoromethyl)-1,3,4-oxadiazol-2-yl)-2,6-diazaspiro[3.4]octan-2-yl)((S)-2,2-dimethylcyclopropyl)methanone (57 mg, 0.12 mmol) in MeCN (2 mL) was added Na₂CO₃ (38 mg, 0.36 mmol) and the mixture stirred at room temperature for 30 min. 7-chlorothiazolo[4,5-d]pyrimidine (20 mg, 0.10 mmol) was added and stirring continued overnight. The reaction was filtered through celite and the filtrate concentrated. The residue was purified by prep-HPLC to afford (8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)((S)-2,2-dimethylcyclopropyl)methanone (5.6 mg, 7.6%) as a white solid. LCMS m/z=606.1 [M+H]⁺; ¹H NMR (400 MHz, DMSO-d₆) δ 9.72 (s, 1H), 8.53 (s, 1H), 8.01-7.97 (m, 1H), 7.85 (t, J=7.2 Hz, 1H), 7.69 (dd, J=8.6, 2.3 Hz, 1H), 4.41-3.87 (m, 9H), 1.38-1.25 (m, 1H), 1.12-1.08 (m, 2H), 1.07-1.03 (m, 2H), 0.97 (d, J=22.9 Hz, 2H), 0.85 (s, 1H), 0.71-0.63 (m, 1H).

Table 20: The compounds listed in Table 20 were synthesized from 2-((3,4-dichlorophenyl)difluoromethyl)-5-(2,6-diazaspiro[3.4]octan-8-yl)-1,3,4-oxadiazole according to the procedures outlined for I-205 using the appropriate commercially available reagents and/or intermediates described elsewhere.

TABLE 20

| Example | | | |
|---|---|---|---|
| Number | Compound | ¹HNMR | LCMS |
| I-204 | 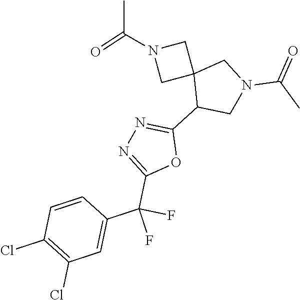 [SMILES: CC(=O)N1CC(c2nnc(C(F)(F)c3ccc(Cl)c(Cl)c3)o2)C2(C1)CN(C(C)=O)C2] | ¹H NMR (400 MHz, Methanol-d₄) δ 7.90 (s, 1H), 7.74 (s, 1H), 7.64 (s, 1H), 4.38-3.76 (m, 9H), 2.08 (s, 3H), 1.83 (dd, J = 20.8, 5.8 Hz, 3H). | m/z = 459.1 [M + H]⁺ |
| I-203 | 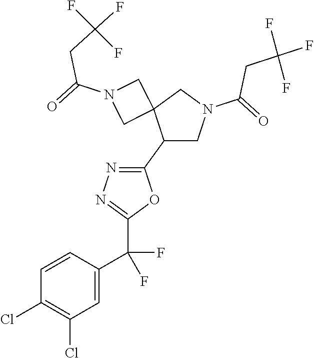 [SMILES: O=C(CC(F)(F)F)N1CC(c2nnc(C(F)(F)c3ccc(Cl)c(Cl)c3)o2)C2(C1)CN(C(=O)CC(F)(F)F)C2] | ¹H NMR (400 MHz, Methanol-d₄) δ 7.92- 7.88 (m, 1H), 7.76 (d, J = 8.4 Hz, 1H), 7.63 (d, J = 8.4 Hz, 1H), 4.43-4.23 (m, 2H), 4.20-3.83 (m, 7H), 3.50-3.39 (m, 2H), 3.26-3.09 (m, 2H). | m/z = 593.1 [M + H]⁺ |

### Synthesis of (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(pyrazin-2-yl)methanone (I-176)

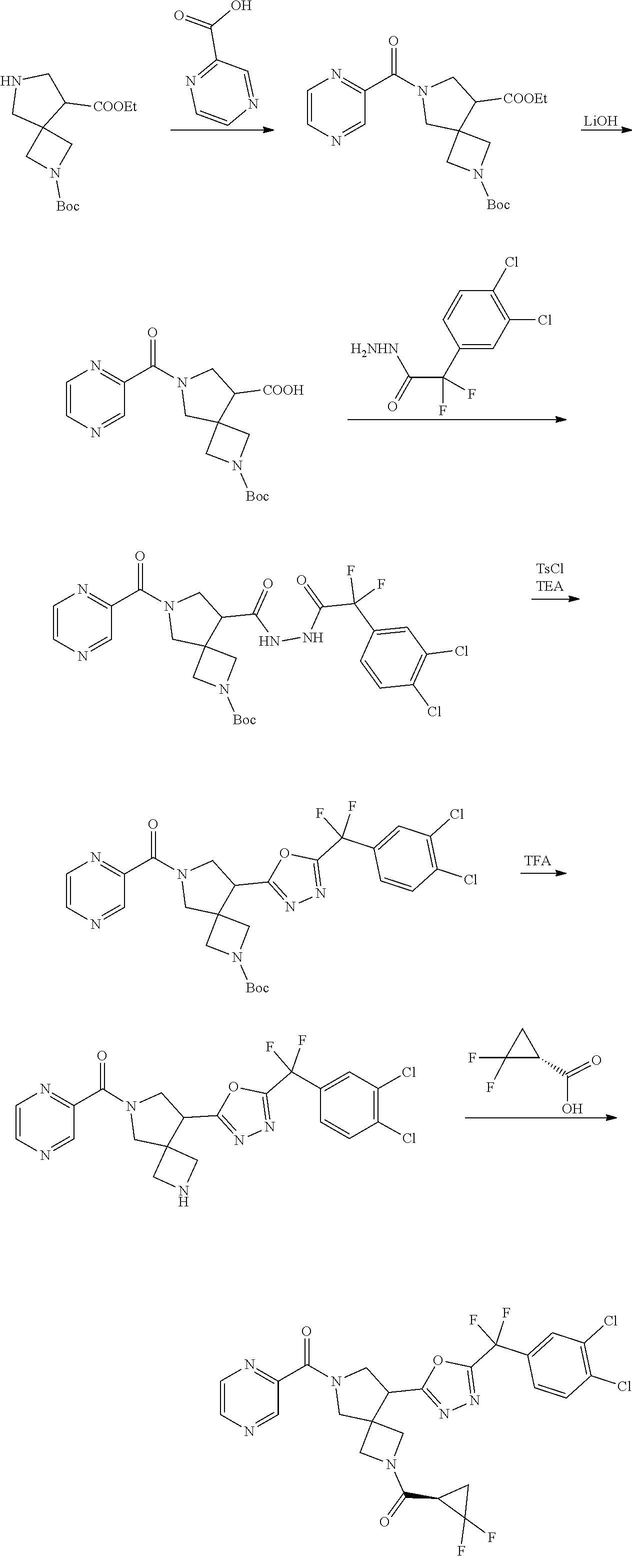

### Step 1: 2-(tert-butyl) 8-ethyl 6-(pyrazine-2-carbonyl)-2,6-diazaspiro[3.4]octane-2,8-dicarboxylate

To a solution of 2-(tert-butyl) 8-ethyl 2,6-diazaspiro[3.4]octane-2,8-dicarboxylate (2 g, 7 mmol) in DMF (10 mL) was added pyrazine-2-carboxylic acid (956 mg, 7.7 mmol), EDCI (2 g, 10.5 mmol), HOBt (1.4 g, 10.5 mmol) and DIPEA (2.7 g, 21 mmol). The mixture was stirred at room temperature overnight then was diluted with water (30 mL) and extracted with EtOAc (50 mL×3). The combined organic layers were washed with brine, dried over Na₂SO₄, filtered and concentrated to afford crude 2-(tert-butyl) 8-ethyl 6-(pyrazine-2-carbonyl)-2,6-diazaspiro[3.4]octane-2,8-dicarboxylate (2 g, 74%) as a yellow oil. LCMS m/z=335.1 [M−56+H]⁺; ¹H NMR (400 MHz, Chloroform-d) δ 9.25-9.15 (m, 1H), 8.71-8.62 (m, 1H), 8.58-8.48 (m, 1H), 4.30-3.77 (m, 10H), 3.20-3.10 (m, 1H), 1.40 (s, 9H), 1.32-1.22 (m, 3H).

### Step 2: 2-(tert-butoxycarbonyl)-6-(pyrazine-2-carbonyl)-2,6-diazaspiro[3.4]octane-8-carboxylic acid

To a solution of 2-(tert-butyl) 8-ethyl 6-(pyrazine-2-carbonyl)-2,6-diazaspiro[3.4]octane-2,8-dicarboxylate (2 g, 5.1 mmol) in a mixture of THF and water (5 mL/1 mL) was added lithium hydroxide monohydrate (642 mg, 15.3 mmol). The mixture was stirred at room temperature for 3 h then concentrated and purified by RP-column to afford 2-(tert-butoxycarbonyl)-6-(pyrazine-2-carbonyl)-2,6-diazaspiro[3.4]octane-8-carboxylic acid (830 mg, 45%) as a white solid. LCMS m/z=362.2 [M+H]⁺; ¹H NMR (400 MHz, CD₃OD) δ 9.07-8.93 (m, 1H), 8.74-8.65 (m, 1H), 8.44 (s, 1H), 4.23-3.51 (m, 8H), 3.15-3.06 (m, 1H), 1.40 (s, 9H).

### Step 3: tert-butyl 8-(2-(2-(3,4-dichlorophenyl)-2,2-difluoroacetyl)hydrazine-1-carbonyl)-6-(pyrazine-2-carbonyl)-2,6-diazaspiro[3.4]octane-2-carboxylate

To a solution of 2-(tert-butoxycarbonyl)-6-(pyrazine-2-carbonyl)-2,6-diazaspiro[3.4]octane-8-carboxylic acid (780 mg, 2.2 mmol) in DMF (5 mL) was added 2-(3,4-dichlorophenyl)-2,2-difluoroacetohydrazide (617 mg, 2.42 mmol), EDCI (633 mg, 3.3 mmol), HOBt (446 mg, 3.3 mmol) and DIPEA (853 mg, 6.6 mmol). The mixture was stirred at room temperature overnight then was diluted with water (30 mL) and extracted with EtOAc (50 mL×3). The combined organic layers were washed with brine, dried over Na₂SO₄, filtered and concentrated. The residue was purified by column chromatography on silica gel (eluent: DCM:MeOH=20:1) to afford tert-butyl 8-(2-(2-(3,4-dichlorophenyl)-2,2-difluoroacetyl)hydrazine-1-carbonyl)-6-(pyrazine-2-carbonyl)-2,6-diazaspiro[3.4]octane-2-carboxylate (163 mg, 13%) as a white solid. LCMS m/z=543.0 [M−56+H]⁺.

### Step 4: tert-butyl 8-(5-((3,4-dichlorophenyl)difluoromethyl)-1,3,4-oxadiazol-2-yl)-6-(pyrazine-2-carbonyl)-2,6-diazaspiro[3.4]octane-2-carboxylate

To a solution of tert-butyl 8-(2-(2-(3,4-dichlorophenyl)-2,2-difluoroacetyl)hydrazine-1-carbonyl)-6-(pyrazine-2-carbonyl)-2,6-diazaspiro[3.4]octane-2-carboxylate (163 mg, 0.27 mmol) in DCM (3 mL) was added TsCl (154 mg, 0.81 mmol) and TEA (82 mg, 0.81 mmol). The reaction mixture was stirred at room temperature for 5 h then was diluted with water (20 mL) and extracted with DCM (50 mL×2). The combined organic layers were washed with brine, dried over Na₂SO₄, filtered and concentrated. The residue was purified by column chromatography on silica gel (eluent: EtOAc:Pet. ether=2:1) to afford tert-butyl 8-(5-((3,4-dichlorophenyl)difluoromethyl)-1,3,4-oxadiazol-2-yl)-6-(pyrazine-2-carbonyl)-2,6-diazaspiro[3.4]octane-2-carboxylate (159 mg, 90%) as a yellow oil. LCMS m/z=525.0 [M+H]⁺; ¹H NMR (400 MHz, CD₃OD) δ 9.07 (s, 1H), 8.72 (s, 1H), 8.69-8.64 (m, 1H), 7.94-7.80 (m, 1H), 7.77-7.69 (m, 1H), 7.66-7.57 (m, 1H), 4.38-3.83 (m, 9H), 1.45-1.39 (m, 9H).

### Step 5: (8-(5-((3,4-dichlorophenyl)difluoromethyl)-1,3,4-oxadiazol-2-yl)-2,6-diazaspiro[3.4]octan-6-yl)(pyrazin-2-yl)methanone

To a solution of tert-butyl 8-(5-((3,4-dichlorophenyl)difluoromethyl)-1,3,4-oxadiazol-2-yl)-6-(pyrazine-2-carbonyl)-2,6-diazaspiro[3.4]octane-2-carboxylate (95 mg, 0.16 mmol) in DCM (3 mL) was added TFA (1.5 mL). The mixture was stirred at room temperature for 3 h then was concentrated to afford (8-(5-((3,4-dichlorophenyl)difluoromethyl)-1,3,4-oxadiazol-2-yl)-2,6-diazaspiro[3.4]octan-6-yl)(pyrazin-2-yl)methanone (152 mg, >99%) as a yellow oil. LCMS m/z=481.0 [M+H]⁺.

### Step 6: (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(pyrazin-2-yl)methanone

To a solution of (R)-2,2-difluorocyclopropane-1-carboxylic acid (22 mg, 0.18 mmol) in DCM (2 mL) was added HATU (91 mg, 0.24 mmol) and DIPEA (62 mg, 0.48 mmol). The mixture was stirred at room temperature for 30 min then (8-(5-((3,4-dichlorophenyl)difluoromethyl)-1,3,4-oxadiazol-2-yl)-2,6-diazaspiro[3.4]octan-6-yl) (pyrazin-2-yl)methanone (152 mg, 0.16 mmol) was added and the reaction stirred another 3 h. The reaction was quenched with water (20 mL) and extracted with DCM (50 mL×2). The combined organic layers were washed with brine, dried over Na₂SO₄, filtered and concentrated. The residue was purified by prep-TLC (eluent: DCM:MeOH=15:1) to afford (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(pyrazin-2-yl)methanone (25 mg, 26%) as a white solid. LCMS m/z=585.0 [M+H]⁺; ¹H NMR (400 MHz, CDCl₃) δ 9.26 (s, 1H), 8.71 (s, 1H), 8.59-8.56 (m, 1H), 7.77 (s, 1H), 7.59 (s, 1H), 7.48-7.47 (m, 1H), 4.48-3.87 (m, 10H), 2.21-2.13 (m, 2H).

Table 21: The compounds listed in Table 21 were synthesized from (8-(5-((3,4-dichlorophenyl)difluoromethyl)-1,3,4-oxadiazol-2-yl)-2,6-diazaspiro[3.4]octan-6-yl)(pyrazin-2-yl)methanone according to the procedures outlined for I-176 using the appropriate commercially available reagents and/or intermediates described elsewhere.

TABLE 21

| Example | | | |
|---|---|---|---|
| Number | Compound | ¹HNMR | LCMS |
| I-175 | 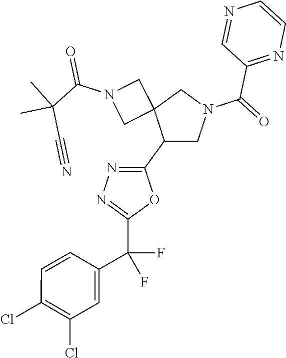 [SMILES: CC(C)(C#N)C(=O)N1CC2(CN(C(=O)c3cnccn3)CC2c2nnc(C(F)(F)c3ccc(Cl)c(Cl)c3)o2)C1] | ¹H NMR (400 MHz, DMSO-d₆) δ 9.01 (s, 1H), 8.79 (t, J = 2.0 Hz, 1H), 8.71 (q, J = 2.2 Hz, 1H), 8.00 (m, 1H), 7.88 (t, J = 7.4 Hz, 1H), 7.71 (m, 1H), 4.71-4.38 (m, 2H), 4.27-3.88 (m, 7H), 1.50-1.34 (m, 6H). | m/z = 576.2 [M + H]⁺ |

### Synthesis of (8-(5-((3,4-dichlorophenyl)difluoromethyl)-1,3,4-oxadiazol-2-yl)-2-((tetrahydrofuran-2-yl)methyl)-2,6-diazaspiro[3.4]octan-6-yl)(thiazol-5-yl)methanone (I-182)

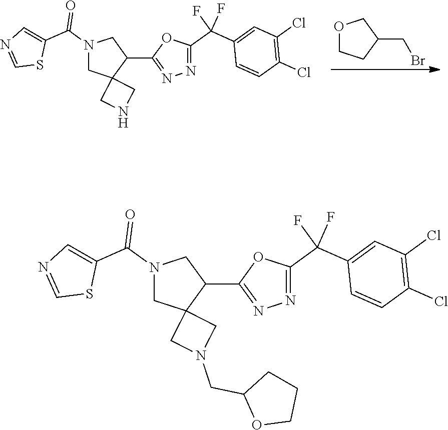 [SMILES: O=C(c1cncs1)N1CC(c2nnc(C(F)(F)c3ccc(Cl)c(Cl)c3)o2)C2(CNC2)C1 ; BrCC1CCOC1 ; O=C(c1cncs1)N1CC(c2nnc(C(F)(F)c3ccc(Cl)c(Cl)c3)o2)C2(CN(CC3CCCO3)C2)C1]

### Step 1: (8-(5-((3,4-dichlorophenyl)difluoromethyl)-1,3,4-oxadiazol-2-yl)-2-((tetrahydrofuran-2-yl)methyl)-2,6-diazaspiro[3.4]octan-6-yl)(thiazol-5-yl)methanone

To a solution of (8-(5-((3,4-dichlorophenyl)difluoromethyl)-1,3,4-oxadiazol-2-yl)-2,6-diazaspiro[3.4]octan-6-yl)(thiazol-5-yl)methanone (50 mg, 0.1 mmol) in DMA (2 mL) was added 3-(bromomethyl)tetrahydrofuran (18 mg, 0.11 mmol) and K₂CO₃ (28 mg, 0.2 mmol). The resulting mixture was heated at 80° C. for 5 h then was cooled to room temperature, filtered and concentrated. The residue obtained was purified by prep-HPLC to afford (8-(5-((3,4-dichlorophenyl)difluoromethyl)-1,3,4-oxadiazol-2-yl)-2-((tetrahydrofuran-2-yl)methyl)-2,6-diazaspiro[3.4]octan-6-yl)(thiazol-5-yl)methanone (4 mg, 7%) as colourless oil. LCMS m/z=570.2 [M+H]⁺; ¹H NMR (400 MHz, CD₃OD) δ 9.20 (s, 1H), 8.38 (s, 1H), 7.91 (s, 1H), 7.75 (d, J=8.4 Hz, 1H), 7.65 (s, 1H), 4.40-3.55 (m, 12H), 2.90 (s, 2H), 2.06-1.82 (m, 3H), 1.52 (s, 1H).

Table 22: The compounds listed in Table 22 were synthesized from (8-(5-((3,4-dichlorophenyl)difluoromethyl)-1,3,4-oxadiazol-2-yl)-2,6-diazaspiro[3.4]octan-6-yl)(thiazol-5-yl)methanone according to the procedures outlined for I-58 using the appropriate commercially available reagents and/or intermediates described elsewhere.

TABLE 22

| Example | | | |
|---|---|---|---|
| Number | Compound | ¹HNMR | LCMS |
| I-196 | 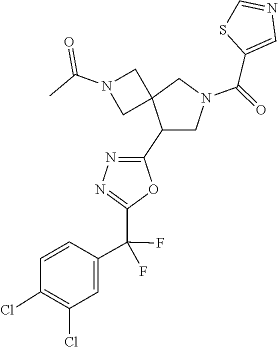 [SMILES: CC(=O)N1CC2(C1)CN(C(=O)c1cncs1)CC2c1nnc(C(F)(F)c2ccc(Cl)c(Cl)c2)o1] | ¹H NMR (400 MHz, CD₃OD) δ 9.15 (s, 1H), 8.36 (d, J = 3.8 Hz, 1H), 7.88 (d, J = 6.7 Hz, 1H), 7.73 (d, J = 8.4 Hz, 1H), 7.62 (d, J = 7.6 Hz, 1H), 4.43-4.22 (m, 4H), 4.20- 3.94 (m, 5H), 1.83 (dd, J = 19.8, 6.4 Hz, 3H). | m/z = 528.1 [M + H]⁺ |
| I-178 | 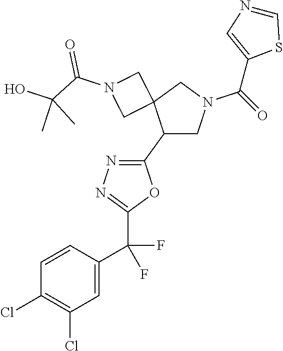 [SMILES: CC(C)(O)C(=O)N1CC2(CN(C(=O)c3cncs3)CC2c2nnc(C(F)(F)c3ccc(Cl)c(Cl)c3)o2)C1] | ¹H NMR (400 MHz, CDCl₃) δ 8.94 (s, 1H), 8.26 (s, 1H), 7.75 (s, 1H), 7.4-7.59 (m, 1H), 7.49- 7.47 (m, 1H), 4.53-3.89 (m, 9H), 1.36 (s, 6H). | m/z = 572.1 [M + H]⁺ |
| I-189 | 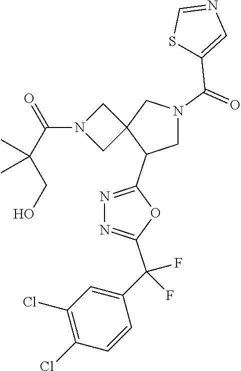 [SMILES: CC(C)(CO)C(=O)N1CC2(CN(C(=O)c3cncs3)CC2c2nnc(C(F)(F)c3ccc(Cl)c(Cl)c3)o2)C1] | ¹H NMR (400 MHz, CDCl₃) δ 8.94 (s, 1H), 8.26 (s, 1H), 7.76 (d, J = 2.2 Hz, 1H), 7.61 (d, J = 8.4 Hz, 1H), 7.49 (d, J = 8.4 Hz, 1H), 4.43-3.81 (m, 9H), 3.47 (d, J = 2.4 Hz, 2H), 1.14 (s, 6H). | m/z = 586.1 [M + H]⁺ |
| I-190 | 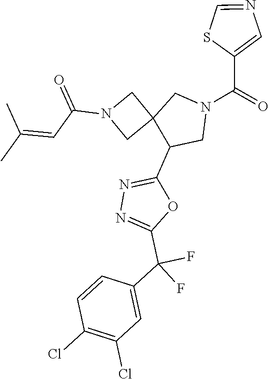 [SMILES: CC(C)=CC(=O)N1CC2(C1)CN(C(=O)c1cncs1)CC2c1nnc(C(F)(F)c2ccc(Cl)c(Cl)c2)o1] | ¹H NMR (400 MHz, Chloroform-d) δ 8.94 (s, 1H), 8.26 (s, 1H), 7.76 (s, 1H), 7.60 (d, J = 8.4 Hz, 1H), 7.53-7.43 (m, 1H), 5.49 (s, 1H), 4.38-3.79 (m, 9H), 2.14 (s, 3H), 1.85 (s, 3H). | m/z = 568.2 [M + H]⁺ |
| I-164 | 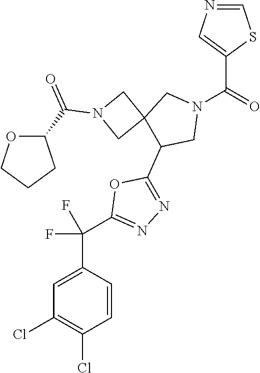 [SMILES: O=C(c1cncs1)N1CC(c2nnc(C(F)(F)c3ccc(Cl)c(Cl)c3)o2)C2(C1)CN(C(=O)[C@@H]1CCCO1)C2] | ¹H NMR (400 MHz, Methanol-d4) δ 9.17 (s, 1H), 8.38 (s, 1H), 7.90 (s, 1H), 7.74 (d, J = 8.8 Hz, 1H), 7.63 (s, 1H), 4.64- 3.66 (m, 12H), 1.90 (s, 4H). | m/z = 584.1 [M + H]⁺ |
| I-157 | 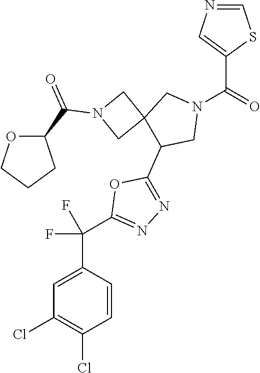 [SMILES: O=C(c1cncs1)N1CC(c2nnc(C(F)(F)c3ccc(Cl)c(Cl)c3)o2)C2(C1)CN(C(=O)[C@H]1CCCO1)C2] | ¹H NMR (400 MHz, CDCl₃) δ 8.94 (s, 1H), 8.26 (s, 1H), 7.77 (s, 1H), 7.63-7.60 (m, 1H), 7.50 (s, 1H), 4.41-3.79 (m, 12H), 2.16-2.09 (m, 2H), 1.88 (s, 2H). | m/z = 584.1 [M + H]⁺ |

### Synthesis of 2-(2-(D-prolyl)-6-(thiazole-5-carbonyl)-2,6-diazaspiro[3.4]octan-8-yl)-5-((3,4-dichlorophenyl)difluoromethyl)-1,3,4-oxadiazole (I-169)

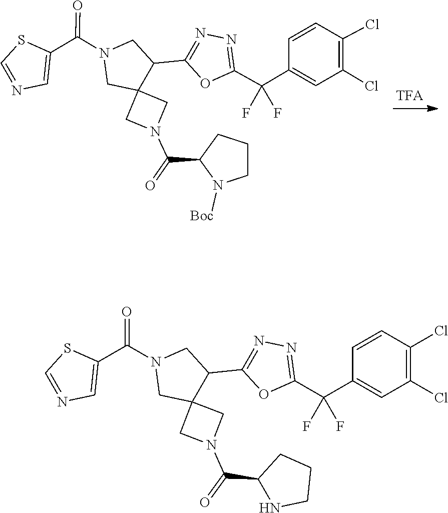 [SMILES: CC(C)(C)OC(=O)N1CCC[C@@H]1C(=O)N1CC2(CN(C(=O)c3cncs3)CC2c2nnc(C(F)(F)c3ccc(Cl)c(Cl)c3)o2)C1 ; O=C(c1cncs1)N1CC(c2nnc(C(F)(F)c3ccc(Cl)c(Cl)c3)o2)C2(C1)CN(C(=O)[C@H]1CCCN1)C2]

tert-butyl (2R)-2-(8-(5-((3,4-dichlorophenyl)difluoromethyl)-1,3,4-oxadiazol-2-yl)-6-(thiazole-5-carbonyl)-2,6-diazaspiro[3.4]octane-2-carbonyl)pyrrolidine-1-carboxylate was synthesized from (8-(5-((3,4-dichlorophenyl)difluoromethyl)-1,3,4-oxadiazol-2-yl)-2,6-diazaspiro[3.4]octan-6-yl)(thiazol-5-yl)methanone according to the procedures outlined for I-58 using the appropriate commercially available reagents and/or intermediates described elsewhere. LCMS m/z=583.0 [M−100]⁺; ¹H NMR (400 MHz, CDCl₃) δ 8.93 (s, 1H), 8.30-8.21 (m, 1H), 7.77 (s, 1H), 7.64-7.58 (m, 1H), 7.53-7.45 (m, 1H), 4.43-3.80 (m, 11H), 3.43 (d, J=36.0 Hz, 3H), 2.01-1.80 (m, 4H), 1.42 (d, J=13.2 Hz, 9H).

### Step 1: 2-(2-(D-prolyl)-6-(thiazole-5-carbonyl)-2,6-diazaspiro[3.4]octan-8-yl)-5-((3,4-dichlorophenyl)difluoromethyl)-1,3,4-oxadiazole

To a solution of tert-butyl (2R)-2-(8-(5-((3,4-dichlorophenyl)difluoromethyl)-1,3,4-oxadiazol-2-yl)-6-(thiazole-5-carbonyl)-2,6-diazaspiro[3.4]octane-2-carbonyl)pyrrolidine-1-carboxylate (40 mg, 0.06 mmol) in DCM (0.5 mL) was added TFA (0.2 mL). The reaction mixture was stirred at room temperature for 1 h then the solvent was removed under reduced pressure. The residue obtained was purified by prep-HPLC to afford 2-(2-(D-prolyl)-6-(thiazole-5-carbonyl)-2,6-diazaspiro[3.4]octan-8-yl)-5-((3,4-dichlorophenyl)difluoromethyl)-1,3,4-oxadiazole (16 mg, 46%) as a white solid. LCMS m/z=583.1 [M+H]⁺; ¹H NMR (400 MHz, CD₃OD) δ 9.17 (s, 1H), 8.46 (s, 1H), 8.38 (s, 1H), 7.90 (d, 1H), 7.75 (d, J=8.1 Hz, 1H), 7.69-7.59 (m, 1H), 4.53-4.00 (m, 10H), 3.42-3.32 (m, 2H), 2.46-2.22 (m, 1H), 2.16-1.66 (m, 3H).

Table 23: The compounds listed in Table 23 were synthesized from (8-(5-((3,4-dichlorophenyl)difluoromethyl)-1,3,4-oxadiazol-2-yl)-2,6-diazaspiro[3.4]octan-6-yl)(thiazol-5-yl)methanone according to the procedures outlined for I-169 using the appropriate commercially available reagents and/or intermediates described elsewhere.

TABLE 23

| Example | | | |
|---|---|---|---|
| Number | Compound | ¹HNMR | LCMS |
| I-173 | 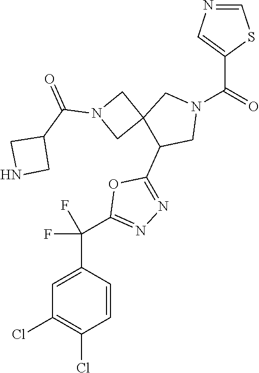 [SMILES: O=C(c1cncs1)N1CC(c2nnc(C(F)(F)c3ccc(Cl)c(Cl)c3)o2)C2(C1)CN(C(=O)C1CNC1)C2] | ¹H NMR (400 MHz, Methanol-d₄) δ 9.17 (s, 1H), 8.45 (s, 1H), 8.36 (s, 1H), 7.93-7.80 (m, 1H), 7.80-7.69 (m, 1H), 7.68- 7.57 (m, 1H), 4.44- 3.94 (m, 13H), 3.79- 3.57 (m, 1H). | m/z = 569.1 [M + H]⁺ |
| I-169 | 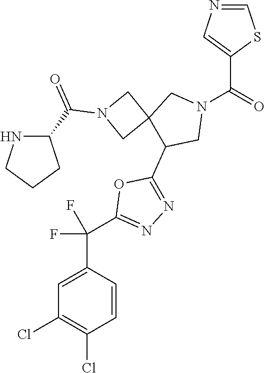 [SMILES: O=C(c1cncs1)N1CC(c2nnc(C(F)(F)c3ccc(Cl)c(Cl)c3)o2)C2(C1)CN(C(=O)[C@@H]1CCCN1)C2] | H NMR (400 MHz, CD₃OD) δ 9.17 (s, 1H), 8.37 (s, 1H), 7.92-7.86 (m, 1H), 7.74 (d, 1H), 7.66-7.60 (m, 1H), 4.53- 4.03 (m, 11H), 3.42- 3.32 (m, 2H), 3.29 (s, 1H), 2.40 (d, J = 8.3 Hz, 1H), 2.12-1.92 (m, 3H). | m/z = 583.2 [M + H]⁺ |

### Synthesis of (8-(5-((3,4-dichlorophenyl)difluoromethyl)-1,3,4-oxadiazol-2-yl)-2-(3,3-dimethyloxirane-2-carbonyl)-2,6-diazaspiro[3.4]octan-6-yl)(thiazol-5-yl)methanone (I-179)

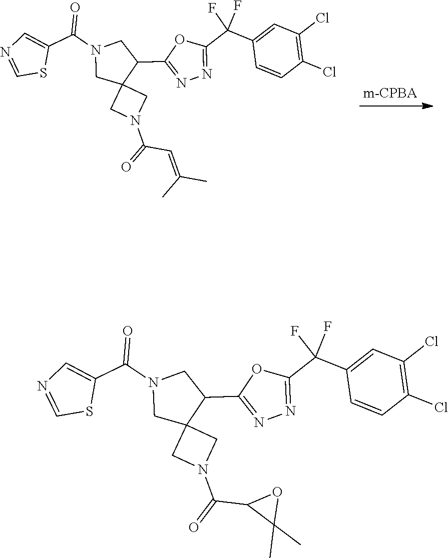 [SMILES: CC(C)=CC(=O)N1CC2(C1)CN(C(=O)c1cncs1)CC2c1nnc(C(F)(F)c2ccc(Cl)c(Cl)c2)o1 ; CC1(C)OC1C(=O)N1CC2(CN(C(=O)c3cncs3)CC2c2nnc(C(F)(F)c3ccc(Cl)c(Cl)c3)o2)C1]

### Step 1: (8-(5-((3,4-dichlorophenyl)difluoromethyl)-1,3,4-oxadiazol-2-yl)-2-(3,3-dimethyloxirane-2-carbonyl)-2,6-diazaspiro[3.4]octan-6-yl)(thiazol-5-yl)methanone

To a solution of 1-(8-(5-((3,4-dichlorophenyl)difluoromethyl)-1,3,4-oxadiazol-2-yl)-6-(thiazole-5-carbonyl)-2,6-diazaspiro[3.4]octan-2-yl)-3-methylbut-2-en-1-one (80.0 mg, 0.141 mmol) in DCM (3 mL) was added m-CPBA (61 mg, 0.352 mmol). The mixture was stirred at room temperature overnight then was diluted with water (30 mL) and extracted with DCM (70 mL×3). The combined organic layers were dried over Na₂SO₄, filtered and concentrated. The residue was purified by prep-HPLC to afford (8-(5-((3,4-dichlorophenyl)difluoromethyl)-1,3,4-oxadiazol-2-yl)-2-(3,3-dimethyloxirane-2-carbonyl)-2,6-diazaspiro[3.4]octan-6-yl)(thiazol-5-yl)methanone (7.0 mg, 8.5%) as a white solid. LCMS m/z=584.1 [M+H]⁺; ¹H NMR (400 MHz, Methanol-d₄) δ 9.18 (s, 1H), 8.39 (s, 1H), 7.91 (s, 1H), 7.75 (d, J=8.4 Hz, 1H), 7.64 (s, 1H), 4.66-3.86 (m, 10H), 1.41-1.34 (m, 3H), 1.32-1.26 (m, 2H), 1.21-1.14 (m, 1H).

### Synthesis of (8-(5-((3,4-dichlorophenyl)difluoromethyl)-1,3,4-oxadiazol-2-yl)-2-(tetrahydrofuran-3-yl)-2,6-diazaspiro[3.4]octan-6-yl)(thiazol-5-yl)methanone (I-154)

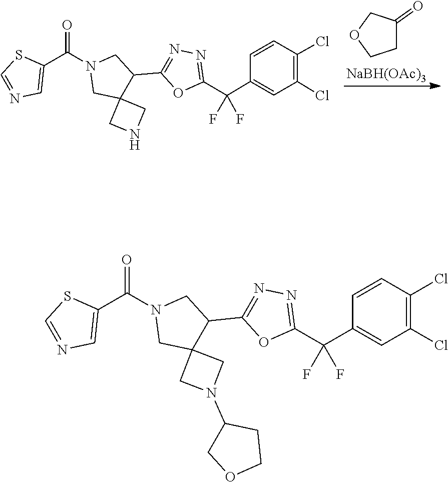

### Step 1: (8-(5-((3,4-dichlorophenyl)difluoromethyl)-1,3,4-oxadiazol-2-yl)-2-(tetrahydrofuran-3-yl)-2,6-diazaspiro[3.4]octan-6-yl)(thiazol-5-yl)methanone

To a solution of (8-(5-((3,4-dichlorophenyl)difluoromethyl)-1,3,4-oxadiazol-2-yl)-2,6-diazaspiro[3.4]octan-6-yl)(thiazol-5-yl)methanone (80 mg, 0.16 mmol) in DCM (2 mL) was added dihydrofuran-3(2H)-one (41 mg, 0.48 mmol) and NaBH(OAc)₃ (68 mg, 0.32 mmol). The mixture was stirred at room temperature overnight then was concentrated under reduced pressure and purified by prep-HPLC to afford (8-(5-((3,4-dichlorophenyl)difluoromethyl)-1,3,4-oxadiazol-2-yl)-2-(tetrahydrofuran-3-yl)-2,6-diazaspiro[3.4]octan-6-yl)(thiazol-5-yl)methanone (27 mg, 29%) as a white solid. LCMS m/z=556.1 [M+H]⁺; ¹H NMR (400 MHz, CDCl₃) δ 8.94 (s, 1H), 8.27 (s, 1H), 8.04 (s, 1H), 7.79 (s, 1H), 7.62-7.60 (m, 1H), 7.53-7.51 (m, 1H), 4.28-3.18 (m, 15H), 1.91-1.72 (m, 1H).

### Synthesis of (8-(5-((3,4-dichlorophenyl)difluoromethyl)-1,3,4-oxadiazol-2-yl)-2-(pyridazin-3-yl)-2,6-diazaspiro[3.4]octan-6-yl)(thiazol-5-yl)methanone (I-187)

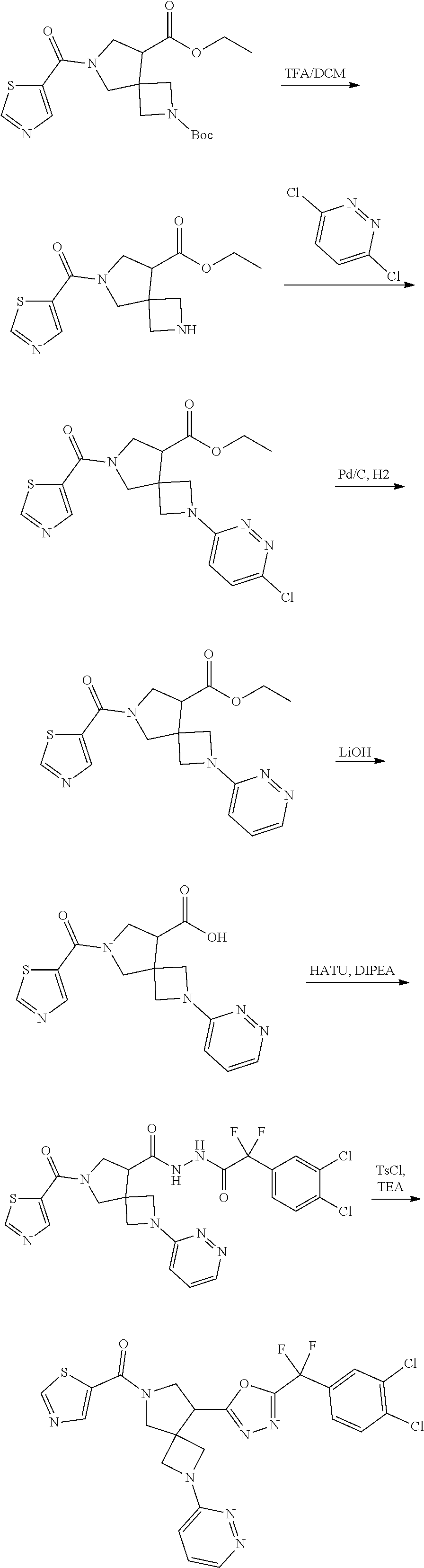

### Step 1: ethyl 6-(thiazole-5-carbonyl)-2,6-diazaspiro[3.4]octane-8-carboxylate

To a solution of 2-(tert-butyl) 8-ethyl 6-(thiazole-5-carbonyl)-2,6-diazaspiro[3.4]octane-2,8-dicarboxylate (300 mg, 0.76 mmol) in DCM (4 mL) was added TFA (1 mL). The reaction mixture was stirred at room temperature for 2 h then the solvent was removed under reduce pressure to afford ethyl 6-benzyl-2,6-diazaspiro[3.4]octane-8-carboxylate (224 mg, 100%) which was used in the next step.

### Step 2: ethyl 2-(6-chloropyridazin-3-yl)-6-(thiazole-5-carbonyl)-2,6-diazaspiro[3.4]octane-8-carboxylate

To a solution of ethyl 6-(thiazole-5-carbonyl)-2,6-diazaspiro[3.4]octane-8-carboxylate (224 mg, 0.75 mmol) in MeCN (3 mL) was added sodium carbonate (239 mg, 2.25 mmol) and 7-chlorothiazolo[4,5-d]pyrimidine (113 mg, 0.75 mmol). The mixture was heated at reflux for 6 h then was concentrated and purified by prep-TLC (eluent: DCM:MeOH=20:1) to afford ethyl 2-(6-chloropyridazin-3-yl)-6-(thiazole-5-carbonyl)-2,6-diazaspiro[3.4]octane-8-carboxylate (300 mg, 97%) as a yellow solid. LCMS m/z=408 [M+H]⁺; ¹H NMR (400 MHz, DMSO-d6) δ 9.31-9.22 (m, 1H), 8.43-8.34 (m, 1H), 8.04 (s, 1H), 7.57-7.48 (m, 1H), 7.00-6.90 (m, 1H), 4.25-3.96 (m, 8H), 3.92-3.74 (m, 2H), 3.56-3.44 (m, 1H), 1.15-1.03 (m, 3H).

### Step 3: ethyl 2-(pyridazin-3-yl)-6-(thiazole-5-carbonyl)-2,6-diazaspiro[3.4]octane-8-carboxylate

To a solution of ethyl 2-(6-chloropyridazin-3-yl)-6-(thiazole-5-carbonyl)-2,6-diazaspiro[3.4]octane-8-carboxylate (300 mg, 0.74 mmol) in MeOH (4 mL) was added 10% Pd/C (150 mg). The reaction mixture was stirred at room temperature under a H₂ atmosphere for 14 h. The catalyst was removed by filtration through celite and the filtrate concentrated. The residue obtained was purified by prep-TLC (eluent: DCM:MeOH=20:1) to afford ethyl 2-(pyridazin-3-yl)-6-(thiazole-5-carbonyl)-2,6-diazaspiro[3.4]octane-8-carboxylate (150 mg, 55% yield) as a yellow solid. LCMS m/z=374.1 [M+H]⁺; ¹H NMR (400 MHz, Chloroform-d) 68.92 (s, 1H), 8.62 (d, J=4.4 Hz, 1H), 8.27 (s, 1H), 6.59 (s, 1H), 4.34-3.90 (m, 11H), 3.31 (s, 1H), 1.29-1.22 (m, 3H).

### Step 4: 2-(pyridazin-3-yl)-6-(thiazole-5-carbonyl)-2,6-diazaspiro[3.4]octane-8-carboxylic acid

To a solution of ethyl 2-(pyridazin-3-yl)-6-(thiazole-5-carbonyl)-2,6-diazaspiro[3.4]octane-8-carboxylate (100 mg, 0.27 mmol) in a mixture of THF, water and EtOH (4 mL/1 mL/1 mL) was added LiOH (23 mg, 0.54 mmol). The reaction mixture was stirred at room temperature for 3 h then was diluted with water (20 mL) and extracted with EtOAc (40 mL). The aqueous layer was collected, acidified to pH˜2 with 1M HCl and extracted with EtOAc (50 mL×3). The combined organic layers were washed with brine, dried over Na₂SO₄, filtered and concentrated to afford 2-(pyridazin-3-yl)-6-(thiazole-5-carbonyl)-2,6-diazaspiro[3.4]octane-8-carboxylic acid (40 mg, 43%) as a white solid. LCMS m/z=346.1 [M+H]⁺; ¹H NMR (400 MHz, Methanol-d4)) δ 9.16 (d, J=4.0 Hz, 1H), 8.54-8.45 (m, 1H), 8.39 (s, 1H), 7.49-7.39 (m, 1H), 6.98-6.86 (m, 1H), 4.41-3.86 (m, 8H), 3.50-3.38 (m, 1H).

### Step 5: N′-(2-(3,4-dichlorophenyl)-2,2-difluoroacetyl)-2-(pyridazin-3-yl)-6-(thiazole-5-carbonyl)-2,6-diazaspiro[3.4]octane-8-carbohydrazide

To a solution of 2-(pyridazin-3-yl)-6-(thiazole-5-carbonyl)-2,6-diazaspiro[3.4]octane-8-carboxylic acid (20 mg, 0.06 mmol) in DMF (1 mL) was added HATU (23 mg, 0.06 mmol) and the mixture was stirred at room temperature for 30 min. 2-(3,4-dichlorophenyl)-2,2-difluoroacetohydrazide (15 mg, 0.06 mmol) and DIPEA (23 mg, 0.18 mmol) were added and the reaction stirred for another 3 h. The mixture was diluted with water (20 mL) and extracted with DCM (50 mL×3). The combined organic layers were washed with brine, dried over Na₂SO₄, filtered and concentrated. The residue obtained was purified by prep-TLC (eluent: DCM:MeOH=20:1) to afford N′-(2-(3,4-dichlorophenyl)-2,2-difluoroacetyl)-2-(pyridazin-3-yl)-6-(thiazole-5-carbonyl)-2,6-diazaspiro[3.4]octane-8-carbohydrazide (15 mg, 44%) as a white solid. LCMS m/z=582 [M+H]⁺.

### Step 6: (8-(5-((3,4-dichlorophenyl)difluoromethyl)-1,3,4-oxadiazol-2-yl)-2-(pyridazin-3-yl)-2,6-diazaspiro[3.4]octan-6-yl)(thiazol-5-yl)methanone (I-187)

To a solution of N′-(2-(3,4-dichlorophenyl)-2,2-difluoroacetyl)-2-(pyridazin-3-yl)-6-(thiazole-5-carbonyl)-2,6-diazaspiro[3.4]octane-8-carbohydrazide (10 mg, 0.02 mmol) in DCM (1 mL) was added TEA (10 mg, 0.1 mmol) and TsCl (11 mg, 0.06 mmol). The reaction mixture was stirred at room temperature for 2 h then was diluted with water (30 mL), extracted with DCM (50 mL×3). The combined organic layers were washed with brine, dried over Na₂SO₄, filtered and concentrated. The residue obtained was purified by prep-TLC (eluent: DCM:MeOH=20:1) to afford (8-(5-((3,4-dichlorophenyl)difluoromethyl)-1,3,4-oxadiazol-2-yl)-2-(pyridazin-3-yl)-2,6-diazaspiro[3.4]octan-6-yl)(thiazol-5-yl)methanone I-187 (7 mg, 78%) as a yellow solid. LCMS m/z=564.1 [M+H]⁺; ¹H NMR (400 MHz, Methanol-d4) δ 9.17 (s, 1H), 8.52 (s, 1H), 8.40 (s, 1H), 7.85-7.79 (m, 1H), 7.65-7.59 (m, 1H), 7.57-7.50 (m, 1H), 7.44-7.34 (m, 1H), 6.84-6.77 (m, 1H), 4.43-4.17 (m, 6H), 4.15-4.02 (m, 3H).

### Synthesis of 2-((3,4-dichlorophenyl)difluoromethyl)-5-(2-(pyrimidin-2-yl)-6-(thiazolo[4,5-d]pyrimidin-7-yl)-2,6-diazaspiro[3.4]octan-8-yl)-1,3,4-oxadiazole (I-183)

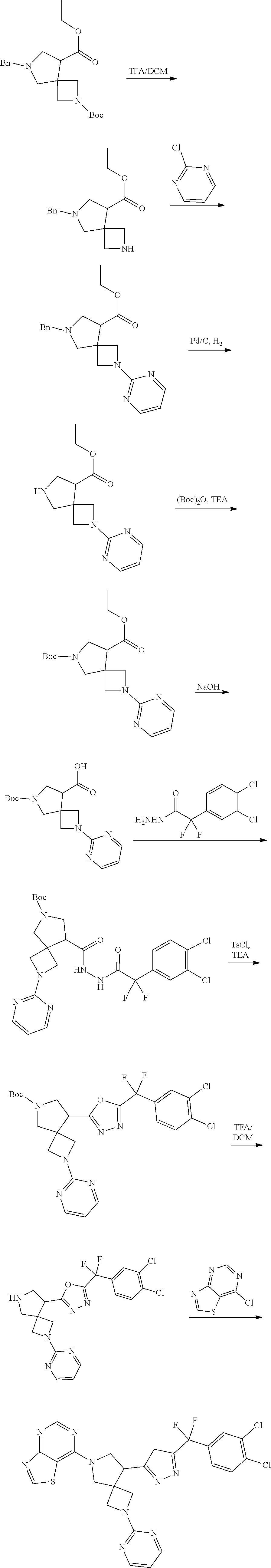

### Step 1: ethyl 6-benzyl-2,6-diazaspiro[3.4]octane-8-carboxylate

To a solution of 2-(tert-butyl) 8-ethyl 6-benzyl-2,6-diazaspiro[3.4]octane-2,8-dicarboxylate (3.1 g, 8.27 mmol) in DCM (10 mL) was added TFA (2.5 mL). The reaction mixture was stirred at room temperature for 1 h then the solvent was removed under vacuum to afford ethyl 6-benzyl-2,6-diazaspiro[3.4]octane-8-carboxylate (2.27 g, 100%) which was used directly in the next step. LCMS m/z=275.2 [M+H]⁺.

### Step 2: ethyl 6-benzyl-2-(pyrimidin-2-yl)-2,6-diazaspiro[3.4]octane-8-carboxylate

To a solution of ethyl 6-benzyl-2,6-diazaspiro[3.4]octane-8-carboxylate (2.27 g, 8 mmol) in MeCN (12 mL) was added Na₂CO₃ (2.5 g, 2.4 mmol). The mixture was stirred at room temperature for 30 min then 2-chloropyrimidine (1 g, 9 mmol) was added and the reaction was stirred overnight. The reaction mixture was filtered through celite and concentrated to afford ethyl 6-benzyl-2-(pyrimidin-2-yl)-2,6-diazaspiro[3.4]octane-8-carboxylate (2.15 g, 77%) as a yellow oil which was used without purification. LCMS m/z=353.2 [M+H]⁺.

### Step 3: ethyl 2-(pyrimidin-2-yl)-2,6-diazaspiro[3.4]octane-8-carboxylate

To a solution of ethyl 6-benzyl-2-(pyrimidin-2-yl)-2,6-diazaspiro[3.4]octane-8-carboxylate (300 mg, 0.85 mmol) in EtOAc (3 mL) was added 10% Pd/C (120 mg). The reaction mixture was stirred under a H₂ atmosphere for 36 h. The catalyst was removed by filtration through celite and the filtrate concentrated to afford ethyl 2-(pyrimidin-2-yl)-2,6-diazaspiro[3.4]octane-8-carboxylate (220 mg, 98%) as a colorless oil. LCMS m/z=263.1 [M+H]⁺; ¹H NMR (400 MHz, DMSO-d₆) δ 8.33 (d, J=4.8 Hz, 2H), 6.66 (t, J=4.7 Hz, 1H), 4.09-3.99 (m, 3H), 3.93-3.88 (m, 2H), 3.86-3.77 (m, 2H), 3.24-3.18 (m, 2H), 3.17 (s, 1H), 3.08-3.05 (m, 1H), 3.04-3.00 (m, 1H), 1.08-0.99 (m, 3H).

### Step 4: 6-(tert-butyl) 8-ethyl 2-(pyrimidin-2-yl)-2,6-diazaspiro[3.4]octane-6,8-dicarboxylate

To a solution of ethyl 2-(pyrimidin-2-yl)-2,6-diazaspiro[3.4]octane-8-carboxylate (220 mg, 0.84 mmol) in DCM (3 mL) was added TEA (127 mg, 1.26 mmol) and (Boc)₂O (219 mg, 1.01 mmol). The reaction mixture was stirred at room temperature for 2 h then was diluted with water (30 mL), extracted with DCM (50 mL×3). The combined organic layers were washed with brine, dried over Na₂SO₄, filtered and concentrated to afford 6-(tert-butyl) 8-ethyl 2-(pyrimidin-2-yl)-2,6-diazaspiro[3.4]octane-6,8-dicarboxylate (240 mg, 80%) as a white solid. LCMS m/z=362.8 [M+H]⁺.

### Step 5: 6-(tert-butoxycarbonyl)-2-(pyrimidin-2-yl)-2,6-diazaspiro[3.4]octane-8-carboxylic acid

To a solution of 6-(tert-butyl) 8-ethyl 2-(pyrimidin-2-yl)-2,6-diazaspiro[3.4]octane-6,8-dicarboxylate (240 mg, 0.66 mmol) in EtOH (2 mL) was added 2M NaOH (2 mL). The reaction was heated at 40° C. for 1 h then was diluted with water (20 mL) and extracted with EtOAc (30 mL). The aqueous layer was collected, acidified with 2 M HCl to pH˜2 and extracted with EtOAc (60 mL×3). The combined organic layers were washed with brine, dried over Na₂SO₄, filtered and concentrated to afford 6-(tert-butoxycarbonyl)-2-(pyrimidin-2-yl)-2,6-diazaspiro[3.4]octane-8-carboxylic acid (110 mg, 50%) as a white solid. LCMS m/z=335.1 [M+H]⁺; ¹H NMR (400 MHz, DMSO-d₆) δ 8.34 (d, J=4.8 Hz, 2H), 6.68 (t, J=4.8 Hz, 1H), 4.16-4.09 (m, 1H), 4.05 (d, J=9.2 Hz, 1H), 3.97-3.90 (m, 2H), 3.63-3.54 (m, 2H), 3.50-3.43 (m, 2H), 3.25-3.18 (m, 1H), 1.41 (s, 9H).

### Step 6: tert-butyl 8-(2-(2-(3,4-dichlorophenyl)-2,2-difluoroacetyl)hydrazine-1-carbonyl)-2-(pyrimidin-2-yl)-2,6-diazaspiro[3.4]octane-6-carboxylate

To a solution of 6-(tert-butoxycarbonyl)-2-(pyrimidin-2-yl)-2,6-diazaspiro[3.4]octane-8-carboxylic acid (110 mg, 0.33 mmol) in DCM (4 mL) was added HATU (125 mg, 0.33 mmol) and DIPEA (128 mg, 0.99 mmol). The mixture was stirred at room temperature for 30 min then 2-(3,4-dichlorophenyl)-2,2-difluoroacetohydrazide (84 mg, 0.33 mmol) was added. The reaction mixture was stirred at room temperature for 2 h then was diluted with water (30 mL) and extracted with DCM (50 mL×3). The combined organic layers were washed with brine, dried over Na₂SO₄, filtered and concentrated. The residue was purified by prep-TLC (eluent: DCM:MeOH=15:1) to afford tert-butyl 8-(2-(2-(3,4-dichlorophenyl)-2,2-difluoroacetyl)hydrazine-1-carbonyl)-2-(pyrimidin-2-yl)-2,6-diazaspiro[3.4]octane-6-carboxylate (180 mg, 95%) as a colorless oil. LCMS m/z=570.8 [M+H]⁺.

### Step 7: tert-butyl 8-(5-((3,4-dichlorophenyl)difluoromethyl)-1,3,4-oxadiazol-2-yl)-2-(pyrimidin-2-yl)-2,6-diazaspiro[3.4]octane-6-carboxylate

To a solution of tert-butyl 8-(2-(2-(3,4-dichlorophenyl)-2,2-difluoroacetyl)hydrazine-1-carbonyl)-2-(pyrimidin-2-yl)-2,6-diazaspiro[3.4]octane-6-carboxylate (180 mg, 0.32 mmol) in DCM (3 mL) was added TEA (162 mg, 1.6 mmol) and TsCl (180 mg, 0.94 mmol). The mixture was stirred at room temperature for 2 h then was diluted with water (30 mL) and extracted with DCM (50 mL×3). The combined organic layers were washed with brine, dried over Na₂SO₄, filtered and concentrated. The residue was purified by prep-TLC (eluent: DCM:MeOH=15:1) to afford tert-butyl 8-(5-((3,4-dichlorophenyl)difluoromethyl)-1,3,4-oxadiazol-2-yl)-2-(pyrimidin-2-yl)-2,6-diazaspiro[3.4]octane-6-carboxylate (97 mg, 55%) as a colorless oil. LCMS m/z=552.8 [M+H]⁺; ¹H NMR (400 MHz, DMSO-d₆) δ 8.33 (d, J=4.8 Hz, 2H), 7.98 (s, 1H), 7.87-7.81 (m, 1H), 7.65 (t, J=6.8 Hz, 1H), 6.69 (t, J=4.8 Hz, 1H), 4.17-4.01 (m, 4H), 3.92-3.90 (m, 1H), 3.76-3.71 (m, 2H), 3.70-3.64 (m, 2H), 1.41 (s, 9H).

### Step 8: 2-((3,4-dichlorophenyl)difluoromethyl)-5-(2-(pyrimidin-2-yl)-2,6-diazaspiro[3.4]octan-8-yl)-1,3,4-oxadiazole

To a solution of tert-butyl 8-(5-((3,4-dichlorophenyl)difluoromethyl)-1,3,4-oxadiazol-2-yl)-2-(pyrimidin-2-yl)-2,6-diazaspiro[3.4]octane-6-carboxylate (90 mg, 0.16 mmol) in DCM (2 mL) was added TFA (0.5 mL) and the reaction stirred at room temperature for 3 h. The solvent was removed under vacuum to afford 2-((3,4-dichlorophenyl)difluoromethyl)-5-(2-(pyrimidin-2-yl)-2,6-diazaspiro[3.4]octan-8-yl)-1,3,4-oxadiazole (74 mg, 100%) which was used directly in the next step. LCMS m/z=452.8 [M+H]⁺.

### Step 9: 2-((3,4-dichlorophenyl)difluoromethyl)-5-(2-(pyrimidin-2-yl)-6-(thiazolo[4,5-d]pyrimidin-7-yl)-2,6-diazaspiro[3.4]octan-8-yl)-1,3,4-oxadiazole

To a solution of 2-((3,4-dichlorophenyl)difluoromethyl)-5-(2-(pyrimidin-2-yl)-2,6-diazaspiro[3.4]octan-8-yl)-1,3,4-oxadiazole (74 mg, 0.16 mmol) in MeCN (2 mL) was added Na₂CO₃ (52 mg, 0.49 mmol). The mixture was stirred at room temperature for 30 min then 7-chlorothiazolo[4,5-d]pyrimidine (27 mg, 0.16 mmol) was added. The reaction was stirred at room temperature overnight then was filtered through celite and the filtrate concentrated. The residue obtained was purified by prep-HPLC to afford 2-((3,4-dichlorophenyl)difluoromethyl)-5-(2-(pyrimidin-2-yl)-6-(thiazolo[4,5-d]pyrimidin-7-yl)-2,6-diazaspiro[3.4]octan-8-yl)-1,3,4-oxadiazole (22 mg, 23%) as a white solid. LCMS m/z=588.0 [M+H]⁺; ¹H NMR (400 MHz, DMSO-d₆) δ 9.73 (s, 1H), 8.55 (s, 1H), 8.35 (d, J=4.8 Hz, 2H), 7.96 (d, 1H), 7.81 (d, J=8.4 Hz, 1H), 7.64 (dd, J=8.5, 2.1 Hz, 1H), 6.71 (t, J=4.8 Hz, 1H), 4.38-4.18 (m, 7H), 4.12-4.07 (m, 1H), 4.06-4.02 (m, 1H).

### Synthesis of (8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)(1-(trifluoromethyl)cyclopropyl)methanone (I-181)

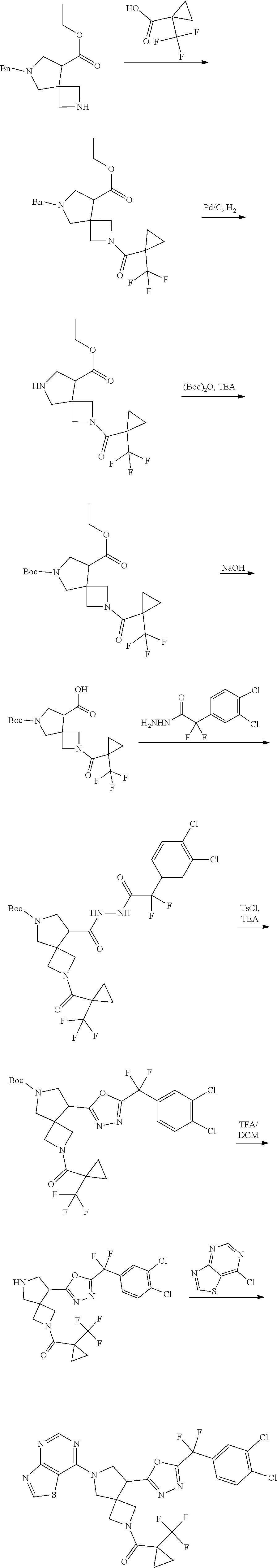

### Step 1: ethyl 6-benzyl-2-(1-(trifluoromethyl)cyclopropane-1-carbonyl)-2,6-diazaspiro[3.4]octane-8-carboxylate

To a solution of 1-(trifluoromethyl)cyclopropane-1-carboxylic acid (416 mg, 2.7 mmol) in DCM (10 mL) was added HATU (1 g, 2.7 mmol) and DIPEA (1.4 g, 10.8 mmol). The mixture was stirred at room temperature for 30 min then ethyl 6-benzyl-2,6-diazaspiro[3.4]octane-8-carboxylate (733 mg, 2.7 mmol) was added. The reaction was stirred for another 2 h then was diluted with water (100 mL) and extracted with DCM (100 mL). The combined organic layers were washed with brine, dried over Na₂SO₄, filtered and concentrated. The residue obtained was purified by column chromatography on silica gel (eluent: DCM:MeOH=50:1) to afford ethyl 6-benzyl-2-(1-(trifluoromethyl)cyclopropane-1-carbonyl)-2,6-diazaspiro[3.4]octane-8-carboxylate (800 mg, 73%) as a yellow oil. LCMS m/z=411.1 [M+H]⁺; ¹H NMR (400 MHz, DMSO-d6) δ 7.52-7.39 (m, 5H), 4.49-3.60 (m, 10H), 3.34-3.20 (m, 2H), 1.29 (t, J=7.2 Hz, 3H), 1.26-1.13 (m, 4H).

### Step 2: ethyl 2-(1-(trifluoromethyl)cyclopropane-1-carbonyl)-2,6-diazaspiro[3.4]octane-8-carboxylate

To a solution of ethyl 6-benzyl-2-(1-(trifluoromethyl)cyclopropane-1-carbonyl)-2,6-diazaspiro[3.4]octane-8-carboxylate (2 g, 4.9 mmol) in EtOAc (16 mL) was added 10% Pd/C (800 mg). The reaction was heated at 40° C. under a H₂ atmosphere for 48 h then the catalyst was removed by filtration through celite and the filtrate concentrated to afford ethyl 2-(1-(trifluoromethyl)cyclopropane-1-carbonyl)-2,6-diazaspiro[3.4]octane-8-carboxylate (1.5 g) which was used in the next step without purification. LCMS m/z=321.1 [M+H]⁺; ¹H NMR (400 MHz, Chloroform-d) δ 4.91-4.03 (m, 7H), 3.69-3.56 (m, 3H), 3.31 (t, J=7.4 Hz, 1H), 1.29 (t, J=7.2 Hz, 3H), 1.26-1.19 (m, 4H).

### Step 3: 6-(tert-butyl) 8-ethyl 2-(1-(trifluoromethyl)cyclopropane-1-carbonyl)-2,6-diazaspiro [3.4]octane-6,8-dicarboxylate

To a solution of ethyl 2-(1-(trifluoromethyl)cyclopropane-1-carbonyl)-2,6-diazaspiro[3.4]octane-8-carboxylate (1.6 g, 4.99 mmol) in DCM (20 mL) was added TEA (1 g, 9.98 mmol) and (Boc)₂O (1.6 g, 7.5 mmol). The reaction was stirred at room temperature for 10 h then was diluted with water (30 mL) and extracted with DCM (50 mL×3). The combined organic layers were washed with brine, dried over Na₂SO₄, filtered and concentrated. The residue obtained was purified by column chromatography on silica gel (eluent: DCM:MeOH=50:1) to afford 6-(tert-butyl) 8-ethyl 2-(1-(trifluoromethyl)cyclopropane-1-carbonyl)-2,6-diazaspiro[3.4]octane-6,8-dicarboxylate (1.5 g, 71%) as a yellow oil. LCMS m/z=365.1 [M+H−56]⁺; ¹H NMR (400 MHz, Chloroform-d) δ 4.51-3.83 (m, 6H), 3.77-3.50 (m, 4H), 3.12-3.01 (m, 1H), 1.46 (s, 9H), 1.27 (t, J=7.2 Hz, 3H), 1.21 (s, 4H).

### Step 4: 6-(tert-butoxycarbonyl)-2-(1-(trifluoromethyl)cyclopropane-1-carbonyl)-2,6-diazaspiro [3.4]octane-8-carboxylic acid

To a solution of 6-(tert-butyl) 8-ethyl 2-(1-(trifluoromethyl)cyclopropane-1-carbonyl)-2,6-diazaspiro[3.4]octane-6,8-dicarboxylate (500 mg, 1.2 mmol) in a mixture of THF, water and EtOH (2.0 mL/0.5 mL/0.5 mL) was added NaOH (96 mg, 2.4 mmol). The reaction mixture was stirred at room temperature for 3 h then was diluted with water (20 mL) and extracted with ether (40 mL). The aqueous layer was collected, acidified to pH˜2 with 1M HCl and extracted with EtOAc (50 mL×3). The combined organic layers were washed with brine, dried over Na₂SO₄, filtered and concentrated to afford 6-(tert-butoxycarbonyl)-2-(1-(trifluoromethyl)cyclopropane-1-carbonyl)-2,6-diazaspiro[3.4]octane-8-carboxylic acid (410 mg, 88%) as a white solid. LCMS m/z=337.1 [M+H−56]⁺; ¹H NMR (400 MHz, DMSO-d6) δ 12.89 (s, 1H), 4.42-3.75 (m, 4H), 3.60-3.38 (m, 4H), 3.24-3.16 (m, 1H), 1.26-1.10 (m, 4H).

### Step 5: tert-butyl 8-(2-(2-(3,4-dichlorophenyl)-2,2-difluoroacetyl)hydrazine-1-carbonyl)-2-(1-(trifluoromethyl)cyclopropane-1-carbonyl)-2,6-diazaspiro[3.4]octane-6-carboxylate

To a solution of 6-(tert-butoxycarbonyl)-2-(1-(trifluoromethyl)cyclopropane-1-carbonyl)-2,6-diazaspiro[3.4]octane-8-carboxylic acid (400 mg, 1 mmol) in DCM (5 mL) was added HATU (380 mg, 1 mmol) and the mixture stirred at room temperature for 30 min. 2-(3,4-dichlorophenyl)-2,2-difluoroacetohydrazide (255 mg, 1 mmol) and DIPEA (387 mg, 3 mmol) were added and the reaction stirred for another 3 h. The reaction was diluted with water (20 mL) and extracted with DCM (50 mL×3). The combined organic layers were washed with brine, dried over Na₂SO₄, filtered and concentrated. The residue obtained was purified by prep-TLC (eluent: DCM:MeOH=20:1) to afford tert-butyl 8-(2-(2-(3,4-dichlorophenyl)-2,2-difluoroacetyl)hydrazine-1-carbonyl)-2-(1-(trifluoromethyl)cyclopropane-1-carbonyl)-2,6-diazaspiro[3.4]octane-6-carboxylate (450 mg, 70%) as a white solid. LCMS m/z=629.1 [M+H]⁺1H NMR (400 MHz, DMSO-d6) δ11.12 (s, 1H), 10.37 (s, 1H), 7.92-7.84 (m, 2H), 7.63 (dd, J=8.5, 2.1 Hz, 1H), 4.45-3.74 (m, 4H), 3.64-3.34 (m, 5H), 3.14-3.07 (m, 1H), 1.23-1.11 (m, 4H).

### Step 6: tert-butyl 8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octane-6-carboxylate

To a solution of tert-butyl 8-(2-(2-(3,4-dichlorophenyl)-2,2-difluoroacetyl)hydrazine-1-carbonyl)-2-(1-(trifluoromethyl) cyclopropane-1-carbonyl)-2,6-diazaspiro[3.4]octane-6-carboxylate (200 mg, 0.3 mmol) in DCM (3 mL) was added TEA (152 mg, 1.5 mmol) and TsCl (172 mg, 0.9 mmol). The reaction mixture was stirred at room temperature for 2 h then was diluted with water (30 mL) and extracted with DCM (50 mL×3). The combined organic layers were washed with brine, dried over Na₂SO₄, filtered and concentrated. The residue obtained was purified by prep-TLC (eluent: DCM:MeOH=20:1) to afford tert-butyl 8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octane-6-carboxylate (190 mg, 98%) as a white solid. LCMS m/z=554.95 [M+H−56]⁺; ¹H NMR (400 MHz, Chloroform-d) 67.77 (d, J=2.2 Hz, 1H), 7.61 (d, J=8.4 Hz, 1H), 7.52-7.46 (m, 1H), 4.42-3.92 (m, 4H), 3.90-3.77 (m, 3H), 3.77-3.65 (m, 2H), 1.47 (s, 10H), 1.23-1.15 (m, 4H).

### Step 7: (8-(5-((3,4-dichlorophenyl)difluoromethyl)-1,3,4-oxadiazol-2-yl)-2,6-diazaspiro[3.4]octan-2-yl)(1-(trifluoromethyl)cyclopropyl)methanone

To a solution of tert-butyl 8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octane-6-carboxylate (80 mg, 0.13 mmol) in DCM (4 mL) was added TFA (1 mL). The reaction mixture was stirred at room temperature for 1 h then the solvent was removed under vacuum to afford (8-(5-((3,4-dichlorophenyl)difluoromethyl)-1,3,4-oxadiazol-2-yl)-2,6-diazaspiro[3.4]octan-2-yl)(1-(trifluoromethyl)cyclopropyl)methanone (41 mg, 100%) which was used without purification. LCMS m/z=511.1 [M+H]⁺.

### Step 8: (8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)(1-(trifluoromethyl)cyclopropyl)methanone

To a solution of (8-(5-((3,4-dichlorophenyl)difluoromethyl)-1,3,4-oxadiazol-2-yl)-2,6-diazaspiro[3.4]octan-2-yl)(1-(trifluoromethyl)cyclopropyl)methanone (67 mg, 0.13 mmol) in MeCN (5 mL) was added sodium carbonate (41 mg, 0.39 mmol) and 7-chlorothiazolo[4,5-d]pyrimidine (22 mg, 0.13 mmol). The mixture was heated at reflux for 6 h then was concentrated under reduced pressure. The residue obtained was purified by prep-HPLC to afford (8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)(1-(trifluoromethyl)cyclopropyl)methanone I-181 (42 mg, 49%) as a yellow solid. LCMS m; z=646.1 [M+H]⁺; ¹H NMR (400 MHz, DMSO-d6) δ 9.73 (s, 1H), 8.53 (s, 1H), 7.98 (d, J=2.2 Hz, 1H), 7.86 (d, J=8.4 Hz, 1H), 7.70 (dd, J=8.4, 2.2 Hz, 1H), 4.54-3.89 (m, 9H), 1.30-1.08 (m, 4H).

### Synthesis of (8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)(2,2-difluoro-1-methylcyclopropyl)methanone (I-174)

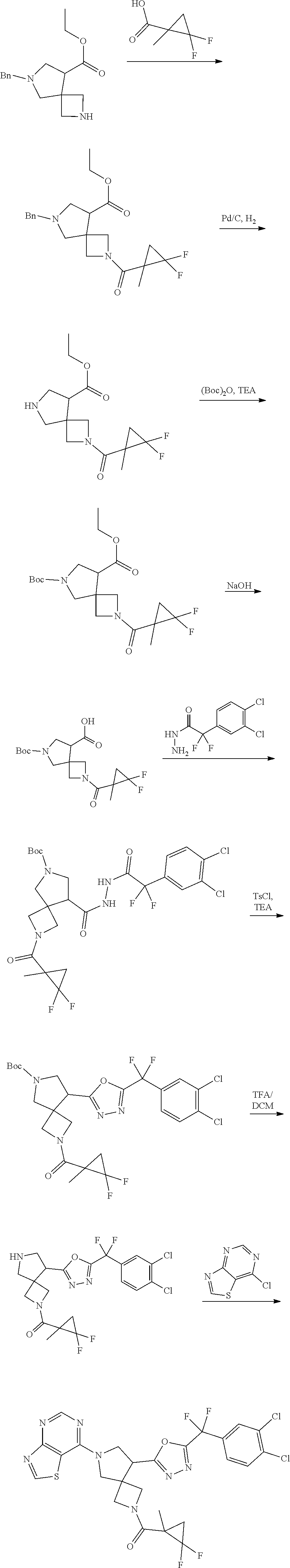

### Step 1: ethyl 6-benzyl-2-(2,2-difluoro-1-methylcyclopropane-1-carbonyl)-2,6-diazaspiro[3.4]octane-8-carboxylate

To a solution of 2,2-difluoro-1-methylcyclopropane-1-carboxylic acid (900 mg, 6.6 mmol) in DCM (15 mL) was added HATU (2.5 g, 6.6 mmol) and DIPEA (3.4 g, 26.5 mmol). The reaction mixture was stirred at room temperature for 30 min then ethyl 6-benzyl-2,6-diazaspiro[3.4]octane-8-carboxylate (1.8 g, 6.6 mmol) was added and the reaction stirred for another 2 h. The mixture was diluted with water (30 mL) and extracted with DCM (50 mL×2). The combined organic layers were washed with brine, dried over Na₂SO₄, filtered and concentrated. The residue was purified by column chromatography on silica gel (eluent: DCM:MeOH=40:1) to afford ethyl 6-benzyl-2-(2,2-difluoro-1-methylcyclopropane-1-carbonyl)-2,6-diazaspiro[3.4]octane-8-carboxylate (1.5 g, 58%) as a yellow oil. LCMS m/z=393.2 [M+H]⁺; ¹H NMR (400 MHz, DMSO-d₆) δ 7.41-7.30 (m, 5H), 4.41-4.05 (m, 4H), 4.02-3.69 (m, 5H), 3.18-2.98 (m, 4H), 1.81 (m, 1H), 1.52-1.44 (m, 1H), 1.34-1.29 (s, 3H), 1.23-1.16 (in, 3H).

### Step 2: ethyl 2-(2,2-difluoro-1-methylcyclopropane-1-carbonyl)-2,6-diazaspiro[3.4]octane-8-carboxylate

To a solution of ethyl 6-benzyl-2-(2,2-difluoro-1-methylcyclopropane-1-carbonyl)-2,6-diazaspiro[3.4]octane-8-carboxylate (1.5 g, 3.82 mmol) in EtOAc (12 mL) was added 10% Pd/C (600 mg). The reaction mixture was stirred under a H₂ atmosphere for 24 h. 50% conversion was observed, the catalyst was removed by filtration through celite and the filtrate concentrated. The residue was redissolved in EtOAc (12 mL) and resubjected to the same conditions for another 24 h. The catalyst was removed by filtration through celite and the filtrate concentrated to afford ethyl 2-(2,2-difluoro-1-methylcyclopropane-1-carbonyl)-2,6-diazaspiro[3.4]octane-8-carboxylate (1.1 g, 95%) which was used directly in the next step. LCMS m/z=303.1 [M+H]⁺; ¹H NMR (400 MHz, DMSO-d₆) δ 4.23 (m, 1H), 4.15-4.07 (m, 2H), 4.05-3.96 (m, 1H), 3.93-3.72 (m, 3H), 3.26 (d, J=9.2 Hz, 2H), 3.16-3.06 (m, 2H), 1.81 (m, 1H), 1.75-1.62 (m, 1H), 1.48 (m, 1H), 1.34 (s, 3H), 1.24-1.16 (m, 3H).

### Step 3: 6-(tert-butyl) 8-ethyl 2-(2,2-difluoro-1-methylcyclopropane-1-carbonyl)-2,6-diazaspiro[3.4]octane-6,8-dicarboxylate

To a solution of ethyl 2-(2,2-difluoro-1-methylcyclopropane-1-carbonyl)-2,6-diazaspiro[3.4]octane-8-carboxylate (1.1 g, 3.64 mmol) in DCM (8 mL) was added TEA (1 mL, 7.28 mmol) and (Boc)₂O (1.3 mL, 5.46 mmol). The reaction was stirred at room temperature for 2 h then was diluted with water (30 mL) and extracted with DCM (50 mL×3). The combined organic layers were washed with brine, dried over Na₂SO₄, filtered and concentrated. The residue obtained was purified by column chromatography on silica gel (eluent: DCM:MeOH=80:1) to afford 6-(tert-butyl) 8-ethyl 2-(2,2-difluoro-1-methylcyclopropane-1-carbonyl)-2,6-diazaspiro[3.4]octane-6,8-dicarboxylate (800 mg, 58%) as a yellow oil. LCMS m/z=347.1 [M+H−56]⁺; ¹H NMR (400 MHz, DMSO-d₆) δ 4.31-4.22 (m, 1H), 4.16-4.03 (m, 3H), 3.99-3.69 (m, 2H), 3.66-3.53 (m, 1H), 3.46 (d, J=6.4 Hz, 3H), 3.30-3.20 (m, 1H), 1.47-1.42 (m, 1H), 1.40 (s, 9H), 1.34 (dd, J=6.8, 3.2 Hz, 3H), 1.23 (d, J=4.2 Hz, 1H), 1.21-1.15 (m, 3H).

### Step 4: 6-(tert-butoxycarbonyl)-2-(2,2-difluoro-1-methylcyclopropane-1-carbonyl)-2,6-diazaspiro[3.4]octane-8-carboxylic acid

To a solution of 6-(tert-butyl) 8-ethyl 2-(2,2-difluoro-1-methylcyclopropane-1-carbonyl)-2,6-diazaspiro[3.4]octane-6,8-dicarboxylate (750 mg, 1.86 mmol) in a mixture of THF, water and EtOH (2.0 mL/0.5 mL/0.5 mL) was added NaOH (149 mg, 3.73 mmol). The reaction mixture was stirred at room temperature for 2 h then diluted with water (10 mL) and extracted EtOAc (20 mL). The aqueous layer was collected, acidified to pH˜2 with 1M HCl and extracted with EtOAc (50 mL×3). The combined organic layers were washed with brine, dried over Na₂SO₄, filtered and concentrated to afford 6-(tert-butoxycarbonyl)-2-(2,2-difluoro-1-methylcyclopropane-1-carbonyl)-2,6-diazaspiro[3.4]octane-8-carboxylic acid (630 mg, 90%) as a yellow solid. LCMS m/z=319.1 [M+H−56]⁺. ¹H NMR (400 MHz, DMSO-d₆) δ 12.81 (s, 1H), 4.32 (m, 1H), 4.18-3.74 (m, 3H), 3.59 (dd, J=23.2, 10.0 Hz, 1H), 3.45 (m, 3H), 3.26-3.12 (m, 1H), 1.88-1.77 (m, 1H), 1.48 (t, J=6.4 Hz, 1H), 1.40 (s, 9H), 1.33 (s, 3H).

### Step 5: tert-butyl 8-(2-(2-(3,4-dichlorophenyl)-2,2-difluoroacetyl)hydrazine-1-carbonyl)-2-(2,2-difluoro-1-methylcyclopropane-1-carbonyl)-2,6-diazaspiro[3.4]octane-6-carboxylate

To a solution of 6-(tert-butoxycarbonyl)-2-(2,2-difluoro-1-methylcyclopropane-1-carbonyl)-2,6-diazaspiro[3.4]octane-8-carboxylic acid (200 mg, 0.53 mmol) in DCM (3 mL) was added HATU (203 mg, 0.53 mmol) and DIPEA (207 mg, 1.6 mmol). The mixture was stirred at room temperature for 30 min then 2-(3,4-dichlorophenyl)-2,2-difluoroacetohydrazide (136 mg, 0.53 mmol) was added and the reaction stirred for another 2 h. The reaction was diluted with water (30 mL) and extracted with DCM (50 mL×2). The combined organic layers were washed with brine, dried over Na₂SO₄, filtered and concentrated. The residue obtained was purified by prep-TLC (eluent: DCM:MeOH=20:1, v/v) to afford tert-butyl 8-(2-(2-(3,4-dichlorophenyl)-2,2-difluoroacetyl)hydrazine-1-carbonyl)-2-(2,2-difluoro-1-methylcyclopropane-1-carbonyl)-2,6-diazaspiro[3.4]octane-6-carboxylate (300 mg, 92%) as a yellow solid. LCMS m/z=555.0 [M+H−56]⁺. ¹H NMR (400 MHz, DMSO-d₆) δ 11.12 (s, 1H), 10.39 (d, J=24.4 Hz, 1H), 7.91-7.84 (m, 2H), 7.63 (d, J=8.4 Hz, 1H), 4.10 (q, J=5.4 Hz, 2H), 4.05-3.65 (m, 3H), 3.53 (d, J=37.4 Hz, 3H), 3.43-3.37 (m, 1H), 1.84 (s, 1H), 1.46 (s, 1H), 1.39 (s, 9H), 1.35-1.29 (m, 3H).

### Step 6: tert-butyl 8-(5-((3,4-dichlorophenyl)difluoromethyl)-1,3,4-oxadiazol-2-yl)-2-(2,2-difluoro-1-methylcyclopropane-1-carbonyl)-2,6-diazaspiro[3.4]octane-6-carboxylate

To a solution of tert-butyl 8-(2-(2-(3,4-dichlorophenyl)-2,2-difluoroacetyl)hydrazine-1-carbonyl)-2-(2,2-difluoro-1-methylcyclopropane-1-carbonyl)-2,6-diazaspiro[3.4]octane-6-carboxylate (280 mg, 0.46 mmol) in DCM (4 mL) was added TEA (0.32 mL, 2.29 mmol) and TsCl (262 mg, 1.37 mmol). The reaction mixture was stirred at room temperature for 2 h then was diluted with water (30 mL) and extracted with DCM (50 mL×3). The combined organic layers were washed with brine, dried over Na₂SO₄, filtered and concentrated. The residue obtained was purified by column chromatography on silica gel (eluent: DCM:MeOH=100:1) to afford tert-butyl 8-(5-((3,4-dichlorophenyl)difluoromethyl)-1,3,4-oxadiazol-2-yl)-2-(2,2-difluoro-1-methylcyclopropane-1-carbonyl)-2,6-diazaspiro[3.4]octane-6-carboxylate (240 mg, 88%) as a yellow oil. LCMS m/z=537.0 [M+H−56]⁺; ¹H NMR (400 MHz, DMSO-d₆) δ 8.04-7.96 (m, 1H), 7.89 (d, J=8.4 Hz, 1H), 7.70 (dd, J=15.4, 8.2 Hz, 1H), 4.44-4.21 (m, 1H), 4.19-3.99 (m, 2H), 3.98-3.89 (m, 1H), 3.82 (d, 1=14.2 Hz, 1H), 3.70 (dd, J=11.2, 5.8 Hz, 3H), 3.58 (d, J=18.6 Hz, 1H), 1.78 (d, J=23.6 Hz, 1H), 1.46 (t, J=6.6 Hz, 1H), 1.39 (s, 9H), 1.27-1.19 (m, 3H).

### Step 7: (8-(5-((3,4-dichlorophenyl)difluoromethyl)-1,3,4-oxadiazol-2-yl)-2,6-diazaspiro[3.4]octan-2-yl)(2,2-difluoro-1-methylcyclopropyl)methanone

To a solution of tert-butyl 8-(5-((3,4-dichlorophenyl)difluoromethyl)-1,3,4-oxadiazol-2-yl)-2-(2,2-difluoro-1-methylcyclopropane-1-carbonyl)-2,6-diazaspiro[3.4]octane-6-carboxylate (120 mg, 0.2 mmol) in DCM (6 mL) was added TFA (0.5 mL) and the reaction stirred at room temperature for 1 h. The solvent was removed under vacuum to afford (8-(5-((3,4-dichlorophenyl)difluoromethyl)-1,3,4-oxadiazol-2-yl)-2,6-diazaspiro[3.4]octan-2-yl)(2,2-difluoro-1-methylcyclopropyl)methanone (100 mg, 100%) which was used without purification. LCMS m/z=493.0 [M+H]⁺.

### Step 8: (8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)(2,2-difluoro-1-methylcyclopropyl)methanone

To a solution of (8-(5-((3,4-dichlorophenyl)difluoromethyl)-1,3,4-oxadiazol-2-yl)-2,6-diazaspiro[3.4]octan-2-yl)(2,2-difluoro-1-methylcyclopropyl)methanone (100 mg, 0.2 mmol) in CH₃CN (4 mL) was added Na₂CO₃ (65 mg, 0.6 mmol) and the mixture was stirred at room temperature for 30 min. 7-chlorothiazolo[4,5-d]pyrimidine (350 mg, 0.2 mmol) was added and the reaction heated at 70° C. for 2 h. The reaction was cooled, filtered through celite and the filtrate concentrated. The residue obtained was purified by prep-TLC (eluent: DCM:MeOH=20:1, v/v) to afford (8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)(2,2-difluoro-1-methylcyclopropyl)methanone (27 mg, 21%) as a white solid. LCMS m/z=628.2 [M+H]⁺; ¹H NMR (400 MHz, DMSO-d₆) δ 9.73 (s, 1H), 8.54 (s, 1H), 7.98 (dd, J=10.4, 4.6 Hz, 1H), 7.86 (d, J=8.4 Hz, 1H), 7.70 (d, J=8.8 Hz, 1H), 4.53-3.94 (m, 9H), 1.81 (m, 1H), 1.47 (m, 1H), 1.34 (d, J=5.8 Hz, 1H), 1.30-1.22 (m, 2H).

### Synthesis of benzo[d]isoxazol-3-yl(8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)methanone (I-156)

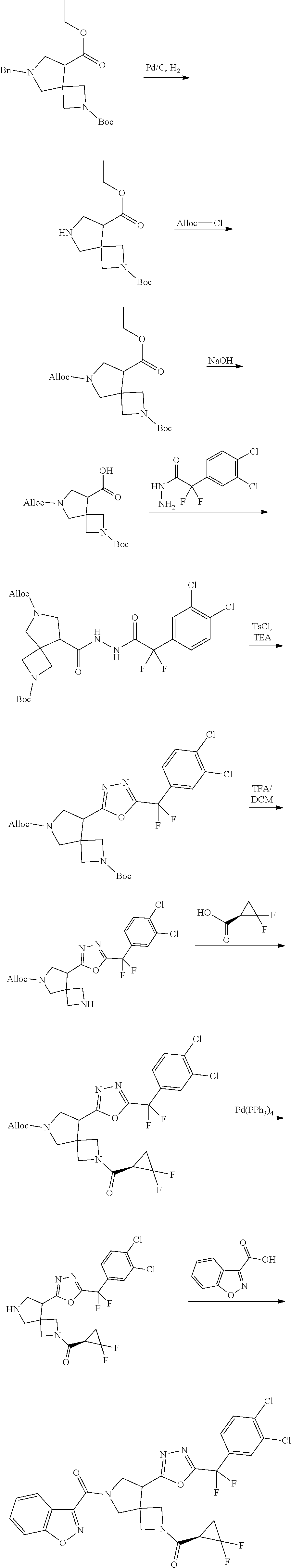

### Step 1: 2-(tert-butyl) 8-ethyl 2,6-diazaspiro[3.4]octane-2,8-dicarboxylate

To a solution of 2-(tert-butyl) 8-ethyl 6-benzyl-2,6-diazaspiro[3.4]octane-2,8-dicarboxylate (5 g, 13.3 mmol) in EtOAc (40 mL) was added 10% Pd/C (2 g). The reaction was heated at 40° C. under a H₂ atmosphere for 48 h. The catalyst was removed by filtration through celite and the filtrate concentrated to afford 2-(tert-butyl) 8-ethyl 2,6-diazaspiro[3.4]octane-2,8-dicarboxylate (3.5 g) which was used directly in the next step. LCMS m/z=285.1 [M+H]⁺.

### Step 2: 6-allyl 2-(tert-butyl) 8-ethyl 2,6-diazaspiro[3.4]octane-2,6,8-tricarboxylate

To a solution of 2-(tert-butyl) 8-ethyl 2,6-diazaspiro[3.4]octane-2,8-dicarboxylate (500 mg, 1.76 mmol) in DCM (10 mL) was added TEA (533 mg, 5.28 mmol) and allyl chloroformate (212 mg, 1.76 mmol). The reaction was stirred at room temperature for 4 h then was diluted with water (30 mL) and extracted with DCM (50 mL×3). The combined organic layers were washed with brine, dried over Na₂SO₄, filtered and concentrated. The residue obtained was purified by column chromatography on silica gel (eluent: Petroleum ether:EtOAc=2:1) to afford 6-allyl 2-(tert-butyl) 8-ethyl 2,6-diazaspiro[3.4]octane-2,6,8-tricarboxylate (400 mg, 62%) as a colourless oil. LCMS m/z=269.1 [M+H−100]⁺; ¹H NMR (400 MHz, Chloroform-d) δ 5.99-5.85 (m, 1H), 5.34-5.26 (m, 1H), 5.21 (d, J=10.4 Hz, 1H), 4.59 (dt, J=5.6, 1.6 Hz, 2H), 4.28-4.13 (m, 2H), 4.02-3.93 (m, 2H), 3.87-3.57 (m, 6H), 3.07 (q, J=6.8, 6.2 Hz, 1H), 1.43 (s, 9H), 1.28 (t, J=7.2 Hz, 3H).

### Step 3: 6-((allyloxy)carbonyl)-2-(tert-butoxycarbonyl)-2,6-diazaspiro[3.4]octane-8-carboxylic acid

To a solution of 6-allyl 2-(tert-butyl) 8-ethyl 2,6-diazaspiro[3.4]octane-2,6,8-tricarboxylate (7 g, 19 mmol) in a mixture of THF, water and EtOH (8 mL/2 mL/2 mL) was added LiOH (1.6 g, 38 mmol). The reaction mixture was stirred at room temperature for 3 h then diluted with water (20 mL) and extracted with ether (40 mL). The aqueous layer was collected, acidified to pH˜2 with 1M HCl and extracted with EtOAc (50 mL×3). The combined organic layers were washed with brine, dried over Na₂SO₄, filtered and concentrated to afford 6-((allyloxy)carbonyl)-2-(tert-butoxycarbonyl)-2,6-diazaspiro[3.4]octane-8-carboxylic acid (6 g, 92%) as a yellow oil. ¹H NMR (400 MHz, Chloroform-d) 65.98-5.86 (m, 1H), 5.34-5.17 (m, 2H), 4.64-4.55 (m, 2H), 4.09-3.94 (m, 2H), 3.89-3.57 (m, 6H), 3.16-3.06 (m, 1H), 1.43 (s, 9H).

### Step 4: 6-allyl 2-(tert-butyl) 8-(2-(2-(3,4-dichlorophenyl)-2,2-difluoroacetyl)hydrazine-1-carbonyl)-2,6-diazaspiro[3.4]octane-2,6-dicarboxylate

To a solution of 6-((allyloxy)carbonyl)-2-(tert-butoxycarbonyl)-2,6-diazaspiro[3.4]octane-8-carboxylic acid (5 g, 14.7 mmol) in DCM (100 mL) was added HATU (5.6 g, 14.7 mmol) and the mixture was stirred at room temperature for 30 min. 2-(3,4-dichlorophenyl)-2,2-difluoroacetohydrazide (3.7 g, 14.7 mmol) and DIPEA (5.7 g, 44.1 mmol) were added and the reaction stirred for another 3 h. The reaction was diluted with water (50 mL) and extracted with DCM (150 mL×3). The combined organic layers were washed with brine, dried over Na₂SO₄, filtered and concentrated. The residue obtained was purified by column chromatography on silica gel (eluent: DCM:MeOH=30:1) to afford 6-allyl 2-(tert-butyl) 8-(2-(2-(3,4-dichlorophenyl)-2,2-difluoroacetyl)hydrazine-1-carbonyl)-2,6-diazaspiro[3.4]octane-2,6-dicarboxylate (8.3 g, 81%) as a white solid. LCMS m/z=477.1 [M+H−100]⁺; ¹H NMR (400 MHz, DMSO-d6) δ 7.91-7.83 (m, 2H), 7.66-7.60 (m, 1H), 5.98-5.84 (m, 1H), 5.33-5.23 (m, 1H), 5.18 (dd, J=10.4, 2.0 Hz, 1H), 4.51 (d, J=5.2 Hz, 2H), 3.95 (d, J=9.2 Hz, 1H), 3.86-3.39 (m, 7H), 3.16-3.06 (m, 1H), 1.36 (s, 9H).

### Step 5: 6-allyl 2-(tert-butyl) 8-(5-((3,4-dichlorophenyl)difluoromethyl)-1,3,4-oxadiazol-2-yl)-2,6-diazaspiro[3.4]octane-2,6-dicarboxylate

To a solution of 6-allyl 2-(tert-butyl) 8-(2-(2-(3,4-dichlorophenyl)-2,2-difluoroacetyl)hydrazine-1-carbonyl)-2,6-diazaspiro[3.4]octane-2,6-dicarboxylate (2.9 g, 5 mmol) in DCM (20 mL) was added TEA (2.5 g, 25 mmol) and TsCl (2.86 g, 15 mmol). The reaction was stirred at room temperature for 2 h then was diluted with water (30 mL) and extracted with DCM (50 mL×3). The combined organic layers were washed with brine, dried over Na₂SO₄, filtered and concentrated. The residue obtained was purified by column chromatography on silica gel (eluent: Petroleum ether:EtOAc=2:1) to afford 6-allyl 2-(tert-butyl) 8-(5-((3,4-dichlorophenyl)difluoromethyl)-1,3,4-oxadiazol-2-yl)-2,6-diazaspiro[3.4]octane-2,6-dicarboxylate (2.3 g, 82%) as a yellow oil. LCMS m/z=459 [M+H−100]⁺; ¹H NMR (400 MHz, Chloroform-d) δ 7.78-7.75 (m, 1H), 7.61 (d, J=8.4 Hz, 1H), 7.51-7.47 (m, 1H), 5.99-5.87 (m, 1H), 5.35-5.31 (m, 1H), 5.29-5.20 (m, 2H), 4.60 (d, J=5.6 Hz, 2H), 4.00-3.71 (m, 9H), 1.42 (s, 9H).

### Step 6: allyl 8-(5-((3,4-dichlorophenyl)difluoromethyl)-1,3,4-oxadiazol-2-yl)-2,6-diazaspiro[3.4]octane-6-carboxylate

To a solution of 6-allyl 2-(tert-butyl) 8-(5-((3,4-dichlorophenyl)difluoromethyl)-1,3,4-oxadiazol-2-yl)-2,6-diazaspiro[3.4]octane-2,6-dicarboxylate (300 mg, 0.5 mmol) in DCM (6 mL) was added TFA (2 mL) and the reaction stirred at room temperature for 1 h. The solvent was removed under vacuum to afford allyl 8-(5-((3,4-dichlorophenyl)difluoromethyl)-1,3,4-oxadiazol-2-yl)-2,6-diazaspiro[3.4]octane-6-carboxylate (246 mg, 100%) which was used in the next step without purification. LCMS m/z=459 [M+H]⁺.

### Step 7: allyl 8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octane-6-carboxylate

To a solution of (R)-2,2-difluorocyclopropane-1-carboxylic acid (1 g, 8 mmol) in DCM (50 mL) was added HATU (3.2 g, 8 mmol) and the mixture stirred at room temperature for 30 min. Allyl 8-(5-((3,4-dichlorophenyl)difluoromethyl)-1,3,4-oxadiazol-2-yl)-2,6-diazaspiro[3.4]octane-6-carboxylate (3.7 g, 8 mmol) and DIPEA (4.3 g, 32 mmol) were added and stirring continued for another 3 h. The reaction was diluted with water (50 mL) and extracted with DCM (150 mL×3). The combined organic layers were washed with brine, dried over Na₂SO₄, filtered and concentrated. The residue obtained was purified by RP column (C18, 80 g, 66% MeCN in water) to afford allyl 8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octane-6-carboxylate (2 g, 44%) as a yellow oil. LCMS m/z=563 [M+H]⁺; ¹H NMR (400 MHz, Chloroform-d) δ 7.77 (s, 1H), 7.61 (d, J=8.4 Hz, 1H), 7.53-7.46 (m, 1H), 6.00-5.86 (m, 1H), 5.38-5.20 (m, 2H), 4.61 (d, J=5.6 Hz, 2H), 4.40-4.19 (m, 2H), 4.11-3.69 (m, 7H), 2.27-2.08 (m, 2H), 1.75-1.65 (m, 1H).

### Step 8: (8-(5-((3,4-dichlorophenyl)difluoromethyl)-1,3,4-oxadiazol-2-yl)-2,6-diazaspiro[3.4]octan-2-yl)((R)-2,2-difluorocyclopropyl)methanone

To a solution of allyl 8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octane-6-carboxylate (1 g, 1.8 mmol) in DCM (20 mL) was added triphenylphosphine (118 mg, 0.45 mmol), Tetrakis(triphenylphosphine)palladium (208 mg, 0.2 mmol) and pyrrolidine (154 mg, 2.16 mmol). The reaction was stirred at room temperature for 30 mins then was diluted with water (30 mL) and extracted with DCM (100 mL×3). The combined organic layers were washed with brine, dried over Na₂SO₄, filtered and concentrated. The residue obtained was purified by column chromatography on silica gel (eluent: DCM:MeOH=15:1) to afford (8-(5-((3,4-dichlorophenyl)difluoromethyl)-1,3,4-oxadiazol-2-yl)-2,6-diazaspiro[3.4]octan-2-yl)((R)-2,2-difluorocyclopropyl)methanone (550 mg, 65%) as a yellow oil. LCMS m/z=479.1 [M+H]⁺; ¹H NMR (400 MHz, Chloroform-d) δ 7.81-7.73 (m, 1H), 7.61 (d, J=8.4 Hz, 1H), 7.49 (t, J=8.2 Hz, 1H), 4.34-3.88 (m, 4H), 3.72-3.64 (m, 1H), 3.49-3.32 (m, 4H), 2.28-2.08 (m, 2H), 1.66-1.59 (m, 1H).

### Step 9: benzo[d]isoxazol-3-yl(8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)methanone

To a solution of benzo[d]isoxazole-3-carboxylic acid (13 mg, 0.08 mmol) in DCM (50 mL) was added HATU (30 mg, 0.08 mmol) and the mixture stirred at room temperature for 30 min. (8-(5-((3,4-dichlorophenyl)difluoromethyl)-1,3,4-oxadiazol-2-yl)-2,6-diazaspiro[3.4]octan-2-yl)((R)-2,2-difluorocyclopropyl)methanone (40 mg, 0.08 mmol) and DIPEA (31 mg, 0.24 mmol) were added and stirring continued for another 3 h. The mixture was concentrated and purified by prep-HPLC to afford benzo[d]isoxazol-3-yl(8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)methanone I-156 (26 mg, 50%) as a white solid. LCMS m/z=624.1 [M+H]⁺; ¹H NMR (400 MHz, DMSO-d6) δ 8.10-7.94 (m, 2H), 7.91-7.83 (m, 2H), 7.78-7.64 (m, 2H), 7.55-7.47 (m, 1H), 4.49-3.89 (m, 9H), 2.64-2.56 (m, 1H), 1.94-1.76 (m, 2H).

### Synthesis of 3-(8-(5-((3,4-dichlorophenyl)difluoromethyl)-1,3,4-oxadiazol-2-yl)-6-(thiazol-5-ylmethyl)-2,6-diazaspiro[3.4]octan-2-yl)-2,2-dimethyl-3-oxopropanenitrile (I-159)

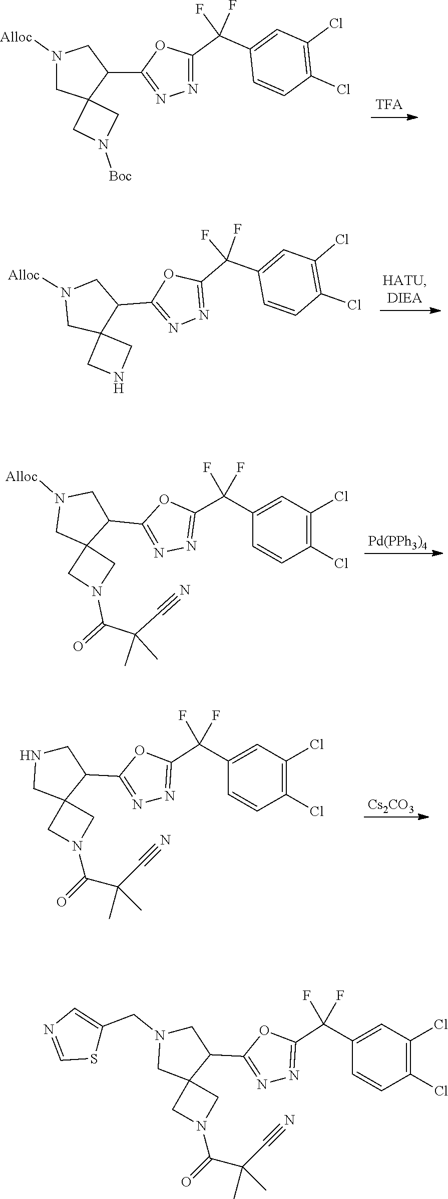

### Step 1: Allyl 8-(5-((3,4-dichloro phenyl)difluoromethyl)-1,3,4-oxadiazol-2-yl)-2,6-diazaspiro[3.4]octane-6-carboxylate

To a solution of 6-allyl 2-(tert-butyl) 8-(5-((3,4-dichlorophenyl)difluoromethyl)-1,3,4-oxadiazol-2-yl)-2,6-diazaspiro[3.4]octane-2,6-dicarboxylate (50 mg, 0.09 mmol) in DCM (1.0 mL) was added TFA and the reaction stirred at room temperature for 2 h. The mixture was concentrated to afford allyl 8-(5-((3,4-dichlorophenyl)difluoromethyl)-1,3,4-oxadiazol-2-yl)-2,6-diazaspiro[3.4]octane-6-carboxylate (41 mg, quant) as a colorless oil. LCMS m/z=459.1 [M+H]⁺.

### Step 2: Allyl 2-(2-cyano-2-methylpropanoyl)-8-(5-((3,4-dichlorophenyl)difluoromethyl)-1,3,4-oxadiazol-2-yl)-2,6-diazaspiro[3.4]octane-6-carboxylate

To a solution of 2-cyano-2-methylpropanoic acid (15 mg, 0.13 mmol) in DCM (1.0 mL) was added HATU (62 mg, 0.16 mmol) and DIPEA (42 mg, 0.32 mmol). The resulting mixture was stirred at room temperature for 30 min then allyl 8-(5-((3,4-dichlorophenyl)difluoromethyl)-1,3,4-oxadiazol-2-yl)-2,6-diazaspiro[3.4]octane-6-carboxylate (50 mg, 0.1 mmol) was added. The reaction was stirred at room temperature for 2 h then was diluted with water (10 mL) and extracted with EtOAc (30 mL×2). The combined organic layers was washed with brine, dried over Na₂SO₄, filtered and concentrated. The residue obtained was purified by prep-TLC (eluent: DCM:MeOH=15:1) to afford allyl 2-(2-cyano-2-methylpropanoyl)-8-(5-((3,4-dichlorophenyl)difluoromethyl)-1,3,4-oxadiazol-2-yl)-2,6-diazaspiro[3.4]octane-6-carboxylate (20 mg, 33%) as a white solid. LCMS m/z=554.1 [M+H]⁺; ¹H NMR (400 MHz, Methanol-d₄) δ 7.90 (d, J=2.1 Hz, 1H), 7.75 (d, J=8.4 Hz, 1H), 7.65-7.61 (m, 1H), 5.96 (ddt, J=16.2, 10.7, 5.4 Hz, 1H), 5.32 (d, J=17.4 Hz, 1H), 5.24-5.19 (m, 1H), 4.63-4.52 (m, 3H), 4.24-3.72 (m, 8H), 1.56-1.42 (m, 6H).

### Step 3: 3-(8-(5-((3,4-dichlorophenyl)difluoromethyl)-1,3,4-oxadiazol-2-yl)-2,6-diazaspiro[3.4]octan-2-yl)-2,2-dimethyl-3-oxopropanenitrile

To a solution of allyl 2-(2-cyano-2-methylpropanoyl)-8-(5-((3,4-dichlorophenyl)difluoromethyl)-1,3,4-oxadiazol-2-yl)-2,6-diazaspiro[3.4]octane-6-carboxylate (100 mg, 0.18 mmol) in DCM (1.0 mL) was added Pd(PPh₃)₄ (21 mg, 18 μmol), PPh₃ (12 mg, 45 μmol) and pyrrolidine (15 mg, 0.21 mmol). The reaction was stirred at room temperature for 2 h then was diluted with water (10 mL) and extracted with EtOAc (30 mL×2). The combined organic layers were washed with brine, dried over Na₂SO₄, filtered and concentrated. The residue obtained was purified by RP-column to afford 3-(8-(5-((3,4-dichlorophenyl)difluoromethyl)-1,3,4-oxadiazol-2-yl)-2,6-diazaspiro[3.4]octan-2-yl)-2,2-dimethyl-3-oxopropanenitrile (35 mg, 42%) as a white solid. LCMS m/z=469.9 [M+H]⁺.

### Step 4: 3-(8-(5-((3,4-dichlorophenyl)difluoromethyl)-1,3,4-oxadiazol-2-yl)-6-(thiazol-5-ylmethyl)-2,6-diazaspiro[3.4]octan-2-yl)-2,2-dimethyl-3-oxopropanenitrile

To a solution of 3-(8-(5-((3,4-dichlorophenyl)difluoromethyl)-1,3,4-oxadiazol-2-yl)-2,6-diazaspiro[3.4]octan-2-yl)-2,2-dimethyl-3-oxopropanenitrile (30 mg, 63.9 μmol) in ACN (1.0 mL) was added 5-(chloromethyl)thiazole (12 mg, 76.7 mol) and Cs₂CO₃ (60 mg, 192 mol). The resulting mixture was heated at 40° C. overnight then diluted with water (10 mL) and extracted with EtOAc (30 mL×2). The combined organic layers were washed with brine, dried over Na₂SO₄, filtered and concentrated. The residue was purified by prep-HPLC to afford 3-(8-(5-((3,4-dichlorophenyl)difluoromethyl)-1,3,4-oxadiazol-2-yl)-6-(thiazol-5-ylmethyl)-2,6-diazaspiro[3.4]octan-2-yl)-2,2-dimethyl-3-oxopropanenitrile (3 mg, 8%) as a yellow solid. LCMS m/z=567.1 [M+H]⁺; ¹H NMR (400 MHz, CD₃OD) δ 8.95 (s, 1H), 7.89 (d, J=2.2 Hz, 1H), 7.81-7.72 (m, 2H), 7.62 (d, J=8.5 Hz, 1H), 4.71 (d, J=9.8 Hz, 1H), 4.40-3.68 (m, 6H), 3.27-3.03 (m, 4H), 1.58-1.39 (m, 6H).

Table 24: The compounds listed in Table 24 were synthesized from 3-(8-(5-((3,4-dichlorophenyl)difluoromethyl)-1,3,4-oxadiazol-2-yl)-2,6-diazaspiro[3.4]octan-2-yl)-2,2-dimethyl-3-oxopropanenitrile according to the procedures outlined for I-159 using the appropriate commercially available reagents and/or intermediates described elsewhere.

TABLE 24

| Example | | | |
|---|---|---|---|
| Number | Compound | ¹HNMR | LCMS |
| I-146 | 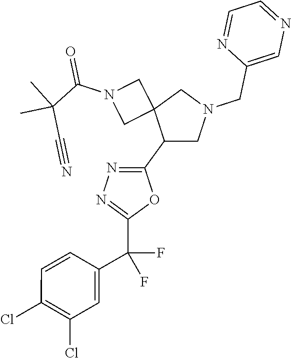 [SMILES: CC(C)(C#N)C(=O)N1CC2(CN(Cc3cnccn3)CC2c2nnc(C(F)(F)c3ccc(Cl)c(Cl)c3)o2)C1] | ¹H NMR (400 MHz, Methanol-d₄) δ 8.70 (d, J = 1.5 Hz, 1H), 8.61- 8.50 (m, 2H), 7.90 (d, J = 2.1 Hz, 1H), 7.75 (d, J = 8.6 Hz, 1H), 7.62 (d, J = 8.6 Hz, 1H), 4.79-3.43 (m, 8H), 3.17 (s, 3H), 1.55-1.40 (m, 6H). | m/z = 561.1 [M + H]⁺ |

### Synthesis of 1-(8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)-3,3-difluoro-2,2-dimethylpropan-1-one (I-170)

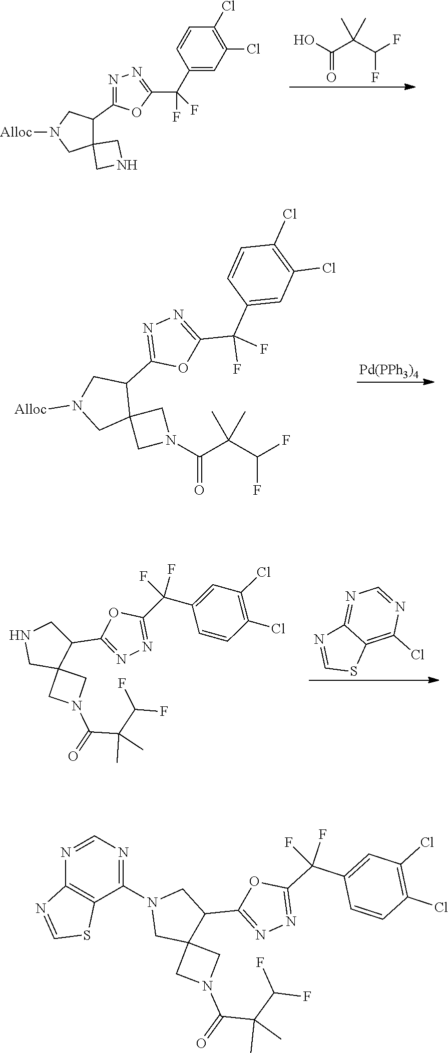

### Step 1: allyl 8-(5-((3,4-dichlorophenyl)difluoromethyl)-1,3,4-oxadiazol-2-yl)-2-(3,3-difluoro-2,2-dimethylpropanoyl)-2,6-diazaspiro[3.4]octane-6-carboxylate

To a solution of 3,3-difluoro-2,2-dimethylpropanoic acid (84 mg, 0.61 mmol) in DCM (3 mL) was added HATU (232 mg, 0.61 mmol) and DIPEA (314 mg, 2.43 mmol) and the mixture stirred at room temperature for 30 min. Allyl 8-(5-((3,4-dichlorophenyl)difluoromethyl)-1,3,4-oxadiazol-2-yl)-2,6-diazaspiro[3.4]octane-6-carboxylate (279 mg, 0.61 mmol) was added and stirring continued for 2 h. The mixture was diluted with water (30 mL) and extracted with DCM (50 mL×3). The combined organic layers were washed with brine, dried over Na₂SO₄, filtered and concentrated. The residue was purified by prep-TLC (eluent: DCM:MeOH=20:1) to afford allyl 8-(5-((3,4-dichlorophenyl)difluoromethyl)-1,3,4-oxadiazol-2-yl)-2-(3,3-difluoro-2,2-dimethylpropanoyl)-2,6-diazaspiro[3.4]octane-6-carboxylate (210 mg, 60%) as a yellow oil. LCMS m/z=579.0 [M+H]⁺; ¹H NMR (400 MHz, DMSO-d₆) δ 8.02 (s, 1H), 7.90 (d, J=8.4 Hz, 1H), 7.74-7.69 (m, 1H), 6.29-6.03 (m, 1H), 6.00-5.87 (m, 1H), 5.35-5.24 (m, 1H), 5.19 (d, J=10.7 Hz, 1H), 4.56-4.51 (m, 2H), 4.23-4.01 (m, 2H), 3.93-3.61 (m, 6H), 3.17 (d, J=5.3 Hz, 1H), 1.21-1.04 (m, 6H).

### Step 2: 1-(8-(5-((3,4-dichlorophenyl)difluoromethyl)-1,3,4-oxadiazol-2-yl)-2,6-diazaspiro[3.4]octan-2-yl)-3,3-difluoro-2,2-dimethylpropan-1-one

To a solution of allyl 8-(5-((3,4-dichlorophenyl)difluoromethyl)-1,3,4-oxadiazol-2-yl)-2-(3,3-difluoro-2,2-dimethylpropanoyl)-2,6-diazaspiro[3.4]octane-6-carboxylate (250 mg, 0.43 mmol), Pd(PPh₃)₄ (50 mg, 0.043 mmol) and PPh₃ (28 mg, 0.108 mmol) in DCM (2 mL) was added pyrrolidine (36 mg, 0.52 mmol). The mixture was stirred at room temperature for 3 h then was diluted with water (30 mL) and extracted with DCM (50 mL×3). The combined organic layers were washed with brine, dried over Na₂SO₄, filtered and concentrated. The residue was purified by column chromatography on silica gel (eluent: DCM:MeOH=50:1 to 20:1) to afford 1-(8-(5-((3,4-dichlorophenyl)difluoromethyl)-1,3,4-oxadiazol-2-yl)-2,6-diazaspiro[3.4]octan-2-yl)-3,3-difluoro-2,2-dimethylpropan-1-one (120 mg, 58%) as a yellow oil. LCMS m/z=495.0 [M+H]⁺; ¹H NMR (400 MHz, DMSO-d₆) δ 8.02 (s, 1H), 7.89 (d, J=8.5 Hz, 1H), 7.71 (dd, J=8.4, 2.2 Hz, 1H), 6.09 (t, J=56.5 Hz, 1H), 4.50-4.28 (m, 1H), 4.25-4.06 (m, 1H), 3.98-3.76 (m, 2H), 3.74-3.62 (m, 1H), 3.27 (s, 1H), 3.19-3.13 (m, 1H), 3.12-3.06 (m, 2H), 1.37-1.32 (m, 1H), 1.19-1.03 (m, 6H).

### Step 3: 1-(8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)-3,3-difluoro-2,2-dimethylpropan-1-one

To a solution of 1-(8-(5-((3,4-dichlorophenyl)difluoromethyl)-1,3,4-oxadiazol-2-yl)-2,6-diazaspiro[3.4]octan-2-yl)-3,3-difluoro-2,2-dimethylpropan-1-one (50 mg, 0.10 mmol) in MeCN (2 mL) was added Na₂CO₃ (32 mg, 0.30 mmol). The mixture was stirred at room temperature for 30 min then 7-chlorothiazolo[4,5-d]pyrimidine (18 mg, 0.10 mmol) was added and stirring continued overnight. The reaction was filtered through celite and the filtrate concentrated. The residue obtained was purified by prep-HPLC to afford 1-(8-(5-((3,4-dichlorophenyl)difluoromethyl)-1,3,4-oxadiazol-2-yl)-6-(thiazolo[4,5-d]pyrimidin-7-yl)-2,6-diazaspiro[3.4]octan-2-yl)-3,3-difluoro-2,2-dimethylpropan-1-one (22 mg, 35%) as a white solid. LCMS m/z=630.1 [M+H]⁺; ¹H NMR (400 MHz, DMSO-d₆) δ 9.73 (s, 1H), 8.53 (s, 1H), 8.03-7.98 (m, 1H), 7.86 (d, J=8.5 Hz, 1H), 7.70 (dd, J=8.5, 2.2 Hz, 1H), 6.12 (t, 1H), 4.63-4.15 (m, 7H), 4.10-3.92 (m, 2H), 1.19-1.06 (m, 6H).

### Synthesis of (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(5-(trifluoromethyl)-1H-pyrazol-4-yl)methanone (I-167)

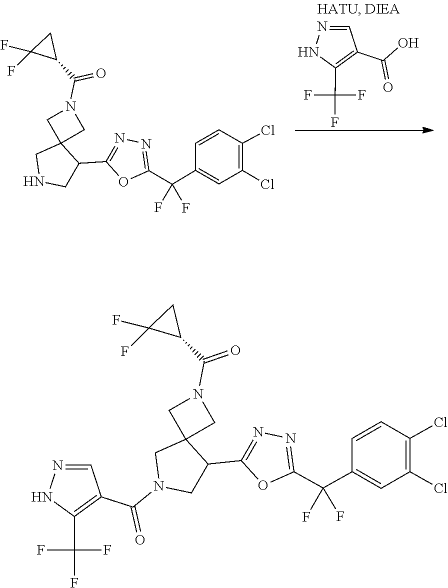 [SMILES: O=C([C@H]1CC1(F)F)N1CC2(CNCC2c2nnc(C(F)(F)c3ccc(Cl)c(Cl)c3)o2)C1 ; O=C(O)c1cn[nH]c1C(F)(F)F ; O=C(c1cn[nH]c1C(F)(F)F)N1CC(c2nnc(C(F)(F)c3ccc(Cl)c(Cl)c3)o2)C2(C1)CN(C(=O)[C@H]1CC1(F)F)C2]

### Step 1: (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(5-(trifluoromethyl)-1H-pyrazol-4-yl)methanone

To a solution of 5-(trifluoromethyl)-1H-pyrazole-4-carboxylic acid (18 mg, 0.1 mmol) in DCM (2 mL) was added HATU (38 mg, 0.1 mmol) and DIPEA (39 mg, 0.3 mmol) and the reaction stirred at room temperature for 10 min. (8-(5-((3,4-dichlorophenyl)difluoromethyl)-1,3,4-oxadiazol-2-yl)-2,6-diazaspiro[3.4]octan-2-yl)((R)-2,2-difluorocyclopropyl)methanone (50 mg, 0.1 mmol) was added and stirring continued for 4 h. The reaction was diluted with water (30 mL) and extracted with DCM (50 mL×3). The combined organic layers were washed with brine, dried over Na₂SO₄, filtered and concentrated. The residue obtained was purified by prep-HPLC to afford (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(5-(trifluoromethyl)-1H-pyrazol-4-yl)methanone (20 mg, 30%) as a white solid. LCMS m/z=641.2 [M+H]⁺; ¹H NMR (400 MHz, CDCl₃) δ 7.77 (s, 2H), 7.63-7.61 (m, 1H), 7.52-7.49 (m, 1H), 4.38-3.87 (m, 9H), 2.14-1.68 (m, 2H).

Table 25: The compounds listed in Table 25 were synthesized from (8-(5-((3,4-dichlorophenyl)difluoromethyl)-1,3,4-oxadiazol-2-yl)-2,6-diazaspiro[3.4]octan-2-yl)((R)-2,2-difluorocyclopropyl)methanone according to the procedures outlined for I-167 using the appropriate commercially available reagents and/or intermediates described elsewhere.

TABLE 25

| Example | | | |
|---|---|---|---|
| Number | Compound | ¹HNMR | LCMS |
| I-166 | 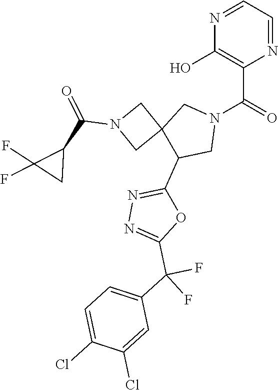 [SMILES: O=C(c1nccnc1O)N1CC(c2nnc(C(F)(F)c3ccc(Cl)c(Cl)c3)o2)C2(C1)CN(C(=O)[C@H]1CC1(F)F)C2] | ¹H NMR (400 MHz, CD₃OD) δ 8.15 (s, 1H), 7.93-7.87 (m, 1H), 7.74 (t, J = 5.8 Hz, 1H), 7.67-7.52 (m, J = 25.6, 11.5 Hz, 3H), 4.58-3.91 (m, 9H), 2.62-2.42 (m, 1H), 1.94 (dd, J = 13.9, 7.5 Hz, 1H), 1.77 (d, J = 13.9 Hz, 1H). | m/z = 601.2 [M + H]⁺ |
| I-168 | 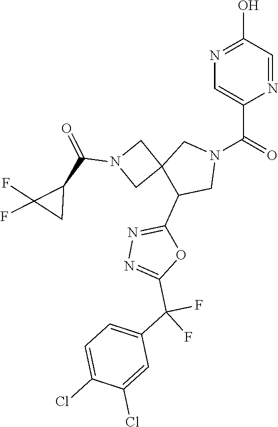 [SMILES: O=C(c1cnc(O)cn1)N1CC(c2nnc(C(F)(F)c3ccc(Cl)c(Cl)c3)o2)C2(C1)CN(C(=O)[C@H]1CC1(F)F)C2] | ¹H NMR (400 MHz, CD₃OD) δ 8.17-7.96 (m, 2H), 7.91-7.80 (m, 1H), 7.70 (d, 1H), 7.66- 7.54 (m, 1H), 4.62-3.89 (m, 9H), 2.65-2.42 (m, 1H), 2.03-1.87 (m, 1H), 1.76 (s, 1H). | m/z = 600.1 [M + H]⁺ |
| I-165 | 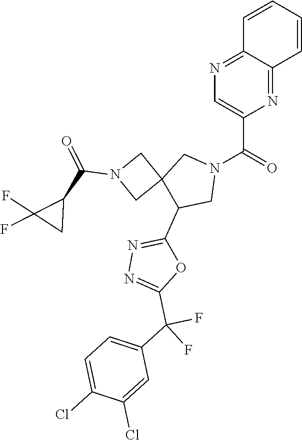 [SMILES: O=C(c1cnc2ccccc2n1)N1CC(c2nnc(C(F)(F)c3ccc(Cl)c(Cl)c3)o2)C2(C1)CN(C(=O)[C@H]1CC1(F)F)C2] | ¹H NMR (400 MHz, Chloroform-d) δ 9.48 (s, 1H), 8.23-8.08 (m, 2H), 7.94-7.83 (m, 2H), 7.81- 7.72 (m, 1H), 7.60 (t, J = 9.4 Hz, 1H), 7.53- 7.44 (m, 1H), 4.71-3.88 (m, 9H), 2.31-2.06 (m, 2H), 1.76-1.60 (m, 1H). | m/z = 635.1 [M + H]⁺ |
| I-160 | 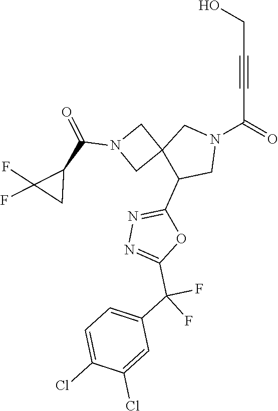 [SMILES: O=C(C#CCO)N1CC(c2nnc(C(F)(F)c3ccc(Cl)c(Cl)c3)o2)C2(C1)CN(C(=O)[C@H]1CC1(F)F)C2] | ¹H NMR (400 MHz, Chloroform-d) δ 7.76 (s, 1H), 7.62 (d, J = 8.4 Hz, 1H), 7.53-7.45 (m, 1H), 4.70 (s, 2H),4.45-3.69 (m, 10H), 2.27-2.06 (m, 2H), 1.75-1.66 (m, 1H). | m/z = 561.0 [M + H]⁺ |
| I-152 | 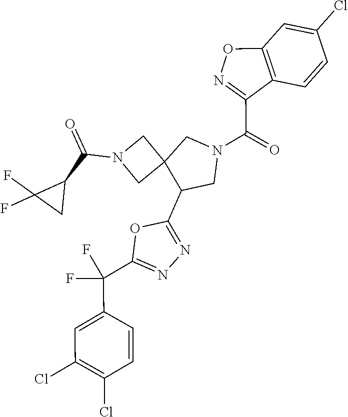 [SMILES: O=C(c1noc2cc(Cl)ccc12)N1CC(c2nnc(C(F)(F)c3ccc(Cl)c(Cl)c3)o2)C2(C1)CN(C(=O)[C@H]1CC1(F)F)C2] | ¹H NMR (400 MHz, DMSO-d₆) δ 8.15 (s, 1H), 8.06 (d, J = 8.6 Hz, 1H), 8.00 (dd, J = 19.8, 6.9 Hz, 1H), 7.87 (t, J = 8.6 Hz, 1H), 7.76-7.66 (m, 1H), 7.57 (d, J = 7.0 Hz, 1H), 4.49-4.19 (m, 5H), 4.15-3.88 (m, 4H), 2.63-2.57 (m, 1H), 1.86 (d, J = 8.8 Hz, 2H). | m/z = 658.0 [M + H]⁺ |
| I-155 | 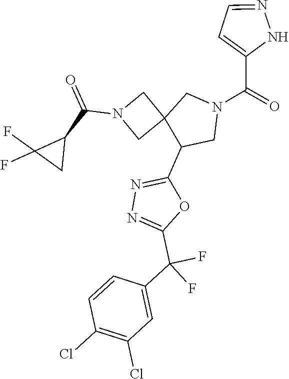 [SMILES: O=C(c1ccn[nH]1)N1CC(c2nnc(C(F)(F)c3ccc(Cl)c(Cl)c3)o2)C2(C1)CN(C(=O)[C@H]1CC1(F)F)C2] | ¹H NMR (400 MHz, Chloroform-d) δ 7.76 (s, 1H), 7.60 (d, J = 13.8 Hz, 2H), 7.48 (s, 1H), 6.88 (s, 1H), 4.55-4.33 (m, 3H), 4.31-4.02 (m, 5H), 3.90- 3.85 (m, 1H), 2.18 (dd, J = 24.2, 12.0 Hz, 2H), 1.67 (s, 1H). | m/z = 573.1 [M + H]⁺ |
| I-163 | 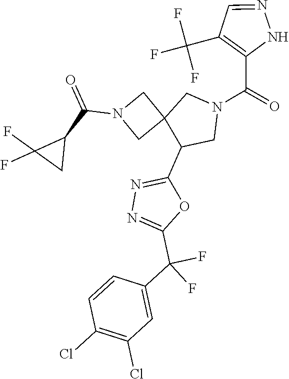 [SMILES: O=C(c1[nH]ncc1C(F)(F)F)N1CC(c2nnc(C(F)(F)c3ccc(Cl)c(Cl)c3)o2)C2(C1)CN(C(=O)[C@H]1CC1(F)F)C2] | ¹H NMR (400 MHz, DMSO-d₆) δ 8.50-8.46 (m, 1H), 8.04-7.96 (m, 1H), 7.90-7.85 (m, 1H), 7.74-7.66 (m, 1H), 4.51- 3.83 (m, 9H), 2.67- 2.57 (m, 1H), 1.90-1.77 (m, 2H). | m/z = 641.1 [M + H]⁺ |
| I-161 | 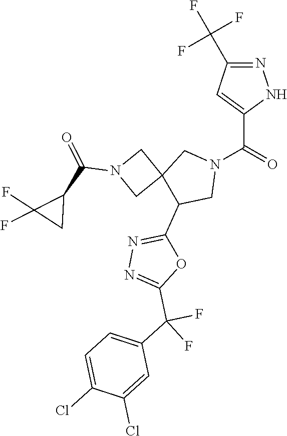 [SMILES: O=C(c1cc(C(F)(F)F)n[nH]1)N1CC(c2nnc(C(F)(F)c3ccc(Cl)c(Cl)c3)o2)C2(C1)CN(C(=O)[C@H]1CC1(F)F)C2] | ¹H NMR (400 MHz, DMSO-d₆) δ 8.05-7.95 (m, 1H), 7.87 (d, J = 8.4 Hz, 1H), 7.75-7.66 (m, 1H), 7.33-7.17 (m, 1H), 4.45-4.11 (m, 5H), 4.09- 3.90 (m, 4H), 2.62 (s, 1H), 1.93-1.77 (m, 2H). | m/z = 641.1 [M + H]⁺ |
| I-149 | 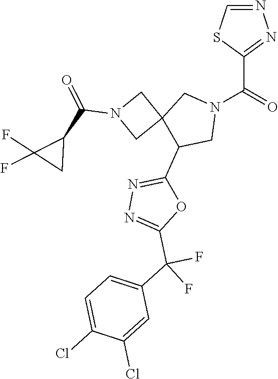 [SMILES: O=C(c1nncs1)N1CC(c2nnc(C(F)(F)c3ccc(Cl)c(Cl)c3)o2)C2(C1)CN(C(=O)[C@H]1CC1(F)F)C2] | ¹H NMR (400 MHz, Chloroform-d) δ 9.27 (d, J = 3.6 Hz, 1H), 7.77 (s, 1H), 7.61 (d, J = 8.4 Hz, 1H), 7.49 (d, J = 8.6 Hz, 1H), 4.84-4.63 (m, 2H), 4.46-3.90 (m, 8H), 2.19 (m, 2H), 1.70 (d, J = 10.8 Hz, 1H). | m/z = 591.0 [M + H]⁺ |
| I-162 | 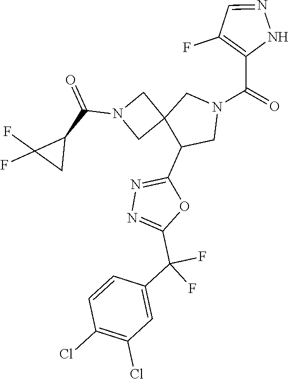 [SMILES: O=C(c1[nH]ncc1F)N1CC(c2nnc(C(F)(F)c3ccc(Cl)c(Cl)c3)o2)C2(C1)CN(C(=O)[C@H]1CC1(F)F)C2] | ¹H NMR (400 MHz, Chloroform-d) δ 7.77 (s, 1H), 7.60 (s, 1H), 7.50 (s, 2H), 4.44-4.03 (m, 8H), 3.88 (s, 1H), 2.17 (m, 2H), 1.68 (s, 1H). | m/z = 591.1 [M + H]⁺ |
| I-172 | 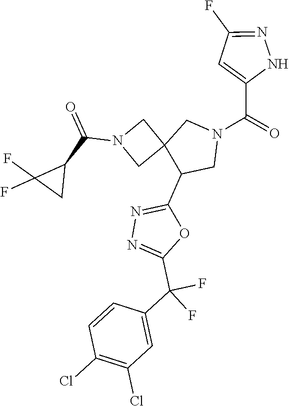 [SMILES: O=C(c1cc(F)n[nH]1)N1CC(c2nnc(C(F)(F)c3ccc(Cl)c(Cl)c3)o2)C2(C1)CN(C(=O)[C@H]1CC1(F)F)C2] | ¹H NMR (400 MHz, Chloroform-d) δ 7.77 (s, 1H), 7.61 (d, J = 8.4 Hz, 1H), 7.53-7.46 (m, 1H), 6.23-6.10 (m, 1H), 4.55- 3.85 (m, 9H), 2.27- 2.08 (m, 2H), 1.76-1.66 (m, 1H). | m/z = 591.1 [M + H]⁺ |
| I-171 | 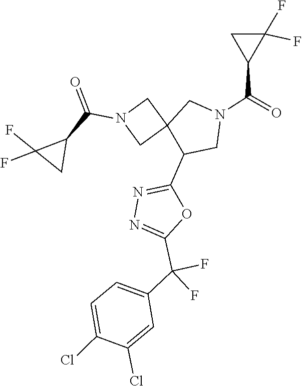 [SMILES: O=C([C@H]1CC1(F)F)N1CC(c2nnc(C(F)(F)c3ccc(Cl)c(Cl)c3)o2)C2(C1)CN(C(=O)[C@H]1CC1(F)F)C2] | ¹H NMR (400 MHz, Chloroform-d) δ 7.79- 7.74 (m, 1H), 7.64-7.58 (m, 1H), 7.53-7.46 (m, 1H), 4.48-4.24 (m, 2H), 4.20-3.80 (m, 7H), 2.52- 2.37 (m, 1H), 2.29- 2.06 (m, 3H), 1.82-1.63 (m, 2H). | m/z = 583.1 [M + H]⁺ |
| I-153 | 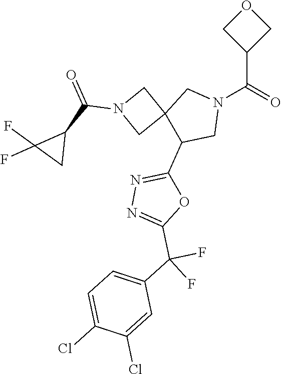 [SMILES: O=C(C1COC1)N1CC(c2nnc(C(F)(F)c3ccc(Cl)c(Cl)c3)o2)C2(C1)CN(C(=O)[C@H]1CC1(F)F)C2] | ¹H NMR (400 MHz, Chloroform-d) δ 7.76 (s, 1H), 7.62 (d, J = 8.4 Hz, 1H), 7.51 (d, J = 10.8 Hz, 1H), 4.91 (d, J = 6.6 Hz, 2H), 4.82 (d, J = 7.8 Hz, 2H), 4.35-4.30 (d, 2H), 4.17-3.92 (m, 5H), 3.81 (s, 2H), 3.64 (d, J = 10.2 Hz, 1H), 2.16 (s, 2H), 1.69 (s, 1H). | m/z = 563.1 [M + H]⁺ |
| I-158 | 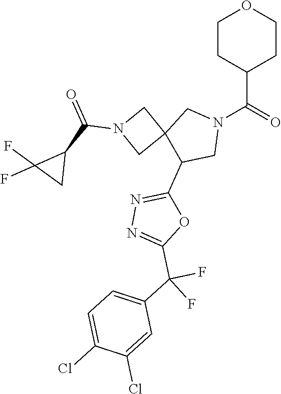 [SMILES: O=C(C1CCOCC1)N1CC(c2nnc(C(F)(F)c3ccc(Cl)c(Cl)c3)o2)C2(C1)CN(C(=O)[C@H]1CC1(F)F)C2] | ¹H NMR (400 MHz, Chloroform-d) δ 7.76 (s, 1H), 7.62 (d, J = 8.4 Hz, 1H), 7.49 (s, 1H), 4.42- 3.69 (m, 11H), 3.43 (s, 2H), 2.57 (s, 1H), 2.18 (s, 2H), 1.91 (d, J = 13.8 Hz, 2H), 1.74-1.59 (m, 3H). | m/z = 591.1 [M + H]⁺ |
| I-138 | 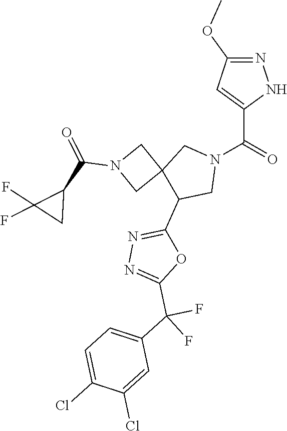 [SMILES: COc1cc(C(=O)N2CC(c3nnc(C(F)(F)c4ccc(Cl)c(Cl)c4)o3)C3(C2)CN(C(=O)[C@H]2CC2(F)F)C3)[nH]n1] | ¹H NMR (400 MHz, DMSO-d₆) δ 12.84- 12.61 (m, 1H), 8.06- 7.96 (m, 1H), 7.91-7.83 (m, 1H), 7.75-7.67 (m, 1H), 6.29-6.03 (m, 1H), 4.42-3.88 (m, 9H), 3.85- 3.76 (m, 3H), 2.66- 2.58 (m, 1H), 1.92-1.77 (m, 2H). | m/z = 603.1 [M + H]⁺ |
| I-125 | 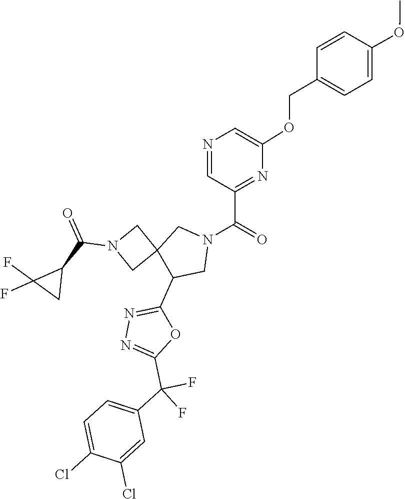 [SMILES: COc1ccc(COc2cncc(C(=O)N3CC(c4nnc(C(F)(F)c5ccc(Cl)c(Cl)c5)o4)C4(C3)CN(C(=O)[C@H]3CC3(F)F)C4)n2)cc1] | ¹H NMR (400 MHz, Chloroform-d) δ 8.78 (d, J = 12.4 Hz, 1H), 8.42 (d, J = 6.6 Hz, 1H), 7.77 (d, J = 11.0 Hz, 1H), 7.60 (s, 1H), 7.43 (d, J = 8.4 Hz, 1H), 7.32 (s, 2H), 6.90 (s, 2H), 5.40-5.31 (m, 2H), 4.39-4.01 (m, 7H), 3.80 (d, J = 2.6 Hz, 5H), 2.14 (s, 2H), 1.68 (s, 1H). | m/z = 719.0 [M − H]⁻ |

### Synthesis of (R)-benzo[c]isoxazol-3-yl(8-(5-((3,4-dichlorophenyl)difluoromethyl)-1,3,4-oxadiazol-2-yl)-2-(2,2-difluorocyclopropane-1-carbonyl)-2,6-diazaspiro[3.4]octan-6-yl)methanone (I-151)

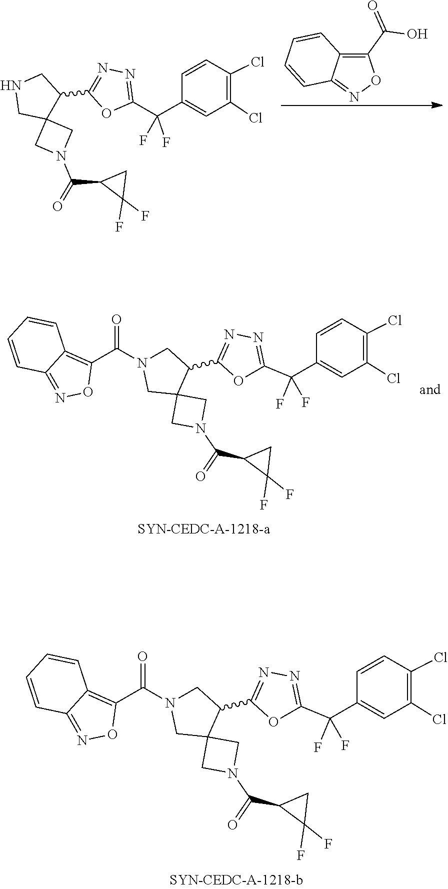

### Step 1: (R)-benzo[c]isoxazol-3-yl(8-(5-((3,4-dichlorophenyl)difluoromethyl)-1,3,4-oxadiazol-2-yl)-2-(2,2-difluorocyclopropane-1-carbonyl)-2,6-diazaspiro[3.4]octan-6-yl)methanone

To a solution of 4-(trifluoromethyl)-1H-pyrazole-5-carboxylic acid (50 mg, 0.10 mmol) in DCM (1 mL) was added HATU (40 mg, 0.1 mmol) and DTPEA (40 mg, 0.3 mmol). The reaction was stirred at room temperature for 30 min then (8-(5-((3,4-dichlorophenyl)difluoromethyl)-1,3,4-oxadiazol-2-yl)-2,6-diazaspiro[3.4]octan-2-yl)((R)-2,2-difluorocyclopropyl)methanone (19 mg, 0.1 mmol) was added and stirring continued for another 2 h. The reaction was diluted with water (15 mL) and extracted with DCM (30 mL×3). The combined organic layers were washed with brine, dried over Na₂SO₄, filtered and concentrated. The residue obtained was purified by prep-HPLC to afford I-151 (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(4-(trifluoromethyl)-1H-pyrazol-5-yl)methanone (3.7 mg, 5%) as a white solid as the first eluting diastereomer. LCMS m/z=624.1 [M+H]⁺; ¹H NMR (400 MHz, DMSO-d₆) δ 8.04-7.94 (m, 2H), 7.89-7.84 (m, 1H), 7.81-7.76 (m, 1H), 7.75-7.66 (m, 1H), 7.54-7.47 (m, 1H), 7.32-7.26 (m, 1H), 4.54-4.24 (m, 5H), 4.14-4.01 (m, 3H), 4.01-3.94 (m, 1H), 2.65-2.55 (m, 1H), 1.91-1.79 (m, 2H). Further elution provided I-151 (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(4-(trifluoromethyl)-1H-pyrazol-5-yl)methanone (6.9 mg, 10%) as a white solid. LCMS m/z=624.1 [M+H]⁺; ¹H NMR (400 MHz, DMSO-d₆) δ 8.00 (dd, J=18.4, 7.8 Hz, 2H), 7.87 (dd, J=8.5, 3.4 Hz, 1H), 7.79 (dd, J=9.1, 5.2 Hz, 1H), 7.72 (d, J=10.9 Hz, 1H), 7.51 (t, J=7.8 Hz, 1H), 7.29 (t, J=7.7 Hz, 1H), 4.55-4.24 (m, 5H), 4.09 (q, J=10.2 Hz, 3H), 4.00-3.91 (m, 1H), 2.60 (d, J=23.1 Hz, 1H), 1.86 (s, 2H).

### Synthesis of (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(3-hydroxy-1H-pyrazol-5-yl)methanone (I-120)

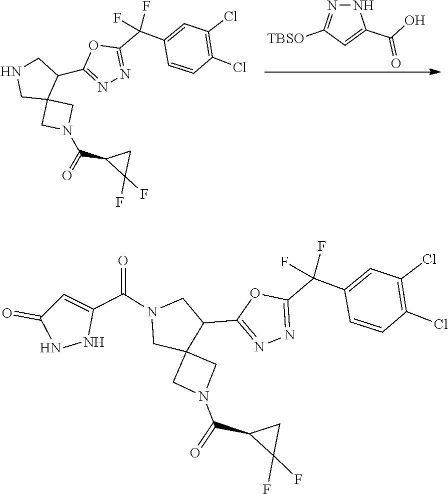 [SMILES: O=C([C@H]1CC1(F)F)N1CC2(CNCC2c2nnc(C(F)(F)c3ccc(Cl)c(Cl)c3)o2)C1 ; CC(C)(C)[Si](C)(C)Oc1cc(C(=O)O)[nH]n1 ; O=C(c1cc(=O)[nH][nH]1)N1CC(c2nnc(C(F)(F)c3ccc(Cl)c(Cl)c3)o2)C2(C1)CN(C(=O)[C@H]1CC1(F)F)C2]

### Step 1: (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(3-hydroxy-1H-pyrazol-5-yl)methanone

To a solution of 3-((tert-butyldimethylsilyl)oxy)-1H-pyrazole-5-carboxylic acid (90 mg, 0.37 mmol), (8-(5-((3,4-dichlorophenyl)difluoromethyl)-1,3,4-oxadiazol-2-yl)-2,6-diazaspiro[3.4]octan-2-yl)((R)-2,2-difluorocyclopropyl)methanone (150 mg, 1.69 mmol) and TEA (170 mg, 1.69 mmol) in dry DMF (2 mL) at 0° C. was added and T₃P (150 mg, 0.5 mmol, 50% in DMF). The mixture was heated at 100° C. under a N₂ atmosphere for 3 h. The mixture was purified by prep-HPLC to afford (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(3-hydroxy-1H-pyrazol-5-yl)methanone (4 mg, 2%) as white solid. LCMS m/z=589.0 [M+H]⁺; ¹HNMR (400 MHz, DMSO-d₆) δ 12.65-12.31 (m, 1H), 8.01 (s, 1H), 7.88 (d, J=8.2 Hz, 1H), 7.75-7.67 (m, 1H), 7.66-7.50 (m, 1H), 5.94-5.84 (m, 1H), 4.45-3.82 (m, 9H), 2.60 (s, 1H), 1.91-1.79 (m, 2H).

### Synthesis of (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(6-hydroxypyrazin-2-yl)methanone (I-112)

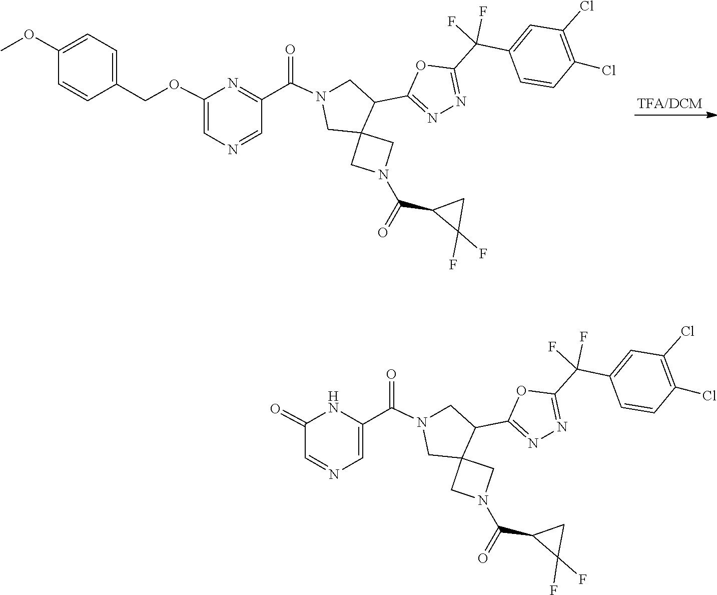 [SMILES: COc1ccc(COc2cncc(C(=O)N3CC(c4nnc(C(F)(F)c5ccc(Cl)c(Cl)c5)o4)C4(C3)CN(C(=O)[C@H]3CC3(F)F)C4)n2)cc1 ; O=C(c1cncc(=O)[nH]1)N1CC(c2nnc(C(F)(F)c3ccc(Cl)c(Cl)c3)o2)C2(C1)CN(C(=O)[C@H]1CC1(F)F)C2]

### Step 1: (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(6-hydroxypyrazin-2-yl)methanone

A solution of (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(6-((4-methoxybenzyl)oxy)pyrazin-2-yl)methanone (37 mg, 0.05 mmol) in a mixture of TFA (1 mL) and DCM (1 mL) was stirred at room temperature for 1 h. The solvent was removed under vacuum and the residue obtained purified by prep-HPLC to afford (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(6-hydroxypyrazin-2-yl)methanone (10.2 mg, 33%) as a white solid. LCMS m/z=601.1 [M+H]⁺; ¹H NMR (400 MHz, DMSO-d6) δ 8.20 (d, J=4.6 Hz, 2H), 8.01 (dd, J=18.2, 6.9 Hz, 1H), 7.88 (s, 1H), 7.73 (d, J=9.8 Hz, 1H), 4.31-3.93 (m, 8H), 3.45-3.43 (m, 1H), 1.86 (d, J=8.8 Hz, 2H).

### Synthesis of (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(morpholino)methanone (I-148)

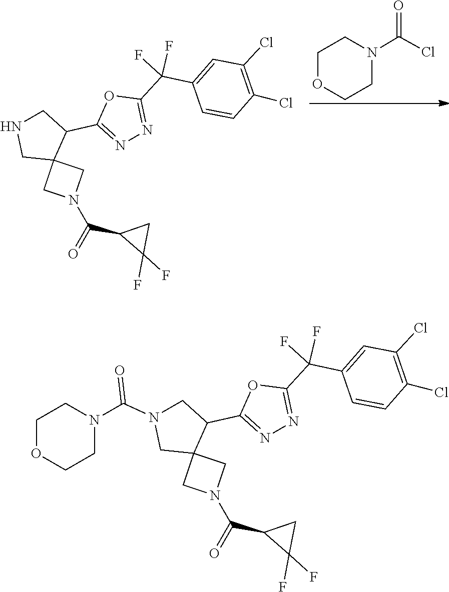

### Step 1: (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(morpholino)methanone

To a solution of (8-(5-((3,4-dichlorophenyl)difluoromethyl)-1,3,4-oxadiazol-2-yl)-2,6-diazaspiro[3.4]octan-2-yl)((R)-2,2-difluorocyclopropyl)methanone 50 mg, 0.10 mmol) in DCM (30 mL) was added DIPEA (17 mg, 0.13 mmol) and morpholine-4-carbonyl chloride (19 mg, 0.13 mmol). The reaction was stirred at room temperature for 10 min then diluted with water (10 mL) and extracted with DCM (20 mL×3). The combined organic layers were washed with brine, dried over Na₂SO₄, filtered and concentrated. The residue obtained was purified by prep-HPLC to afford (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(morpholino)methanone (25 mg, 42%) as a brown solid. LCMS m/z=592.2 [M+H]⁺; ¹H NMR (400 MHz, CDCl₃) δ 7.75 (s, 1H), 7.60 (d, J=8.4 Hz, 1H), 7.52-7.45 (m, 1H), 4.38-4.17 (m, 2H), 4.13-3.74 (m, 7H), 3.71-3.64 (m, 4H), 3.34-3.23 (m, 4H), 2.26-2.05 (m, 2H), 1.73-1.61 (m, 1H).

### Synthesis of (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(piperidin-4-yl)methanone (I-150)

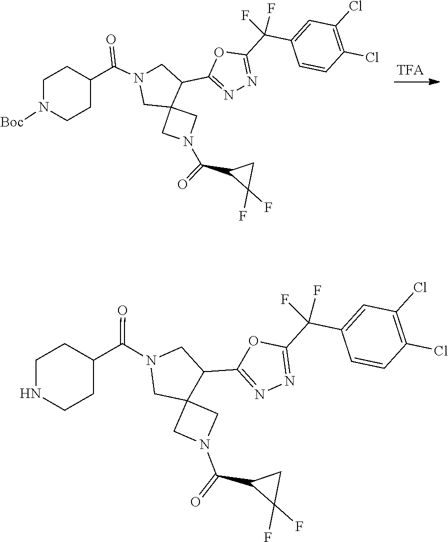

tert-butyl 4-(8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octane-6-carbonyl)piperidine-1-carboxylate was synthesized from (8-(5-((3,4-dichlorophenyl)difluoromethyl)-1,3,4-oxadiazol-2-yl)-2,6-diazaspiro[3.4]octan-2-yl)((R)-2,2-difluorocyclopropyl)methanone according to the procedures outlined for I-167 using the appropriate commercially available reagents and/or intermediates described elsewhere. LCMS m/z=689.8 [M+H]⁺; ¹H NMR (400 MHz, DMSO-d₆) δ 8.03-7.98 (m, 1H), 7.88 (d, J=8.4 Hz, 1H), 7.74-7.68 (m, 1H), 4.42-4.18 (m, 2H), 4.17-4.09 (m, 1H), 4.06-4.01 (m, 1H), 3.99-3.88 (m, 4H), 3.87-3.82 (m, 1H), 3.74-3.67 (m, 1H), 2.65-2.54 (m, 2H), 1.93-1.77 (m, 2H), 1.71-1.53 (m, 2H), 1.39 (s, 1 OH), 1.34 (d, J=6.0 Hz, 1H), 1.22 (d, J=4.0 Hz, 2H).

### Step 1: (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(piperidin-4-yl)methanone

To a solution of tert-butyl 4-(8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octane-6-carbonyl)piperidine-1-carboxylate (50 mg, 0.07 mmol) in DCM (2 mL) was added TFA (0.5 mL) and the reaction stirred at room temperature for 1 h. The solvent was removed under vacuum and the residue obtained purified by prep-HPLC to afford (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(piperidin-4-yl)methanone (31 mg, 52%) as a white solid. LCMS m/z=590.2 [M+H]⁺; ¹H NMR (400 MHz, DMSO-d₆) δ 8.05-7.96 (m, 1H), 7.89 (d, J=8.5 Hz, 1H), 7.75-7.67 (m, 1H), 4.44-4.09 (m, 3H), 4.08-3.86 (m, 4H), 3.86-3.61 (m, 3H), 2.88 (t, J=12.1 Hz, 2H), 2.78-2.69 (m, 1H), 2.65-2.55 (m, 1H), 1.94-1.57 (m, 7H).

Table 26: The compounds listed in Table 26 were synthesized from (8-(5-((3,4-dichlorophenyl)difluoromethyl)-1,3,4-oxadiazol-2-yl)-2,6-diazaspiro[3.4]octan-2-yl)((R)-2,2-difluorocyclopropyl)methanone according to the procedures outlined for I-150 using the appropriate commercially available reagents and/or intermediates described elsewhere

TABLE 26

| Example | | | |
|---|---|---|---|
| Number | Compound | ¹HNMR | LCMS |
| I-147 | 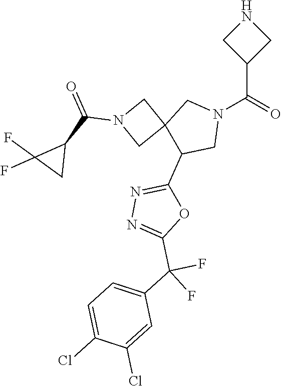 [SMILES: O=C(C1CNC1)N1CC(c2nnc(C(F)(F)c3ccc(Cl)c(Cl)c3)o2)C2(C1)CN(C(=O)[C@H]1CC1(F)F)C2] | ¹H NMR (400 MHz, DMSO-d₆) δ 8.04-7.98 (m, 1H), 7.92-7.87 (m, 1H), 7.75-7.67 (m, 1H), 4.36-4.09 (m, 3H), 4.06- 3.70 (m, 11H), 2.71- 2.60 (m, 1H), 1.92-1.77 (m, 2H). | m/z = 665.2 [M + H]⁺ |

### Synthesis of (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(4-methoxy-1H-pyrazol-5-yl)methanone (I-137)

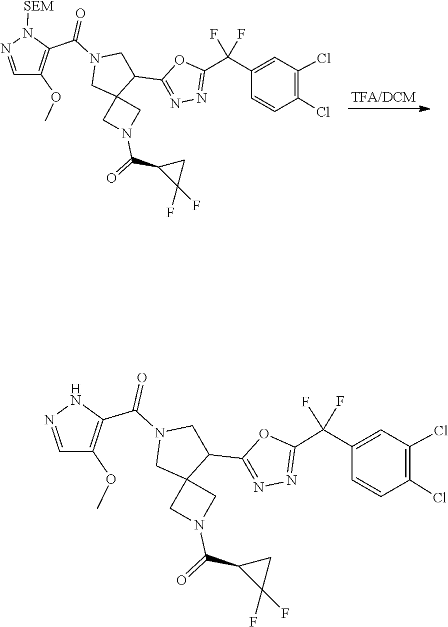 [SMILES: COc1cnn(COCC[Si](C)(C)C)c1C(=O)N1CC(c2nnc(C(F)(F)c3ccc(Cl)c(Cl)c3)o2)C2(C1)CN(C(=O)[C@H]1CC1(F)F)C2 ; COc1cn[nH]c1C(=O)N1CC(c2nnc(C(F)(F)c3ccc(Cl)c(Cl)c3)o2)C2(C1)CN(C(=O)[C@H]1CC1(F)F)C2]

(8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(4-methoxy-1-((2-(trimethylsilyl)ethoxy)methyl)-1H-pyrazol-5-yl)methanone was synthesized from (8-(5-((3,4-dichlorophenyl)difluoromethyl)-1,3,4-oxadiazol-2-yl)-2,6-diazaspiro[3.4]octan-2-yl)((R)-2,2-difluorocyclopropyl)methanone according to the procedures outlined for I-167 using the appropriate commercially available reagents and/or intermediates described elsewhere. LCMS m/z=733.0 [M+H]⁺; ¹H N MNR (400 MHz, DMSO-d₆) δ 8.12 (d, J=9.0 Hz, 1H), 8.01 (m, 1H), 7.88 (d, J=8.6 Hz, 1H), 7.71 (m, 1H), 5.25 (s, 2H), 4.41-4.15 (m, 3H), 3.98-3.80 (m, 9H), 3.55 (m, 2H), 2.67 (m, 1H), 1.85 (s, 2H), 0.84 (d, J=7.6 Hz, 3H), −0.05 (s, 9H).

### Step 1: (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(4-methoxy-1H-pyrazol-5-yl)methanone

To a solution of (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(4-methoxy-1-((2-(trimethylsilyl)ethoxy) methyl)-1H-pyrazol-5-yl)methanone (20 mg, 0.03 mmol) in DCM (2 mL) was added TFA (1 mL). The reaction mixture was stirred at room temperature for 2 h then the solvent was removed under vacuum and the residue purified by prep-TLC (eluent: DCM:MeOH=15:1) to afford (8-(5-((3,4-dichlorophenyl)difluoromethyl)-1,3,4-oxadiazol-2-yl)-2-((R)-2,2-difluorocylopropane-1-carbonyl)-2,6-diazaspiro[3.4]octan-6-yl)(4-methoxy-1H-pyrazol-5-yl)methanone (9.1 mg, 55%) as a white solid. LCMS m/z=603.1 [M+H]; ¹H NMR (400 MHz, Methanol-d₄) δ 7.87 (d, J=22.4 Hz, 2H), 7.74 (d, J=8.6 Hz, 1H), 7.62 (m, 1H), 4.66-4.25 (m, 2H), 4.23-3.87 (m, 9H), 2.55 (s, 1H), 1.87 (m, 2H).

### Synthesis of Azetidin-1-yl(8-(5-((3,4-dichlorophenyl)difluoromethyl)-1,3,4-oxadiazol-2-yl)-6-(thiazole-5-carbonyl)-2,6-diazaspiro[3.4]octan-2-yl)methanone (I-186)

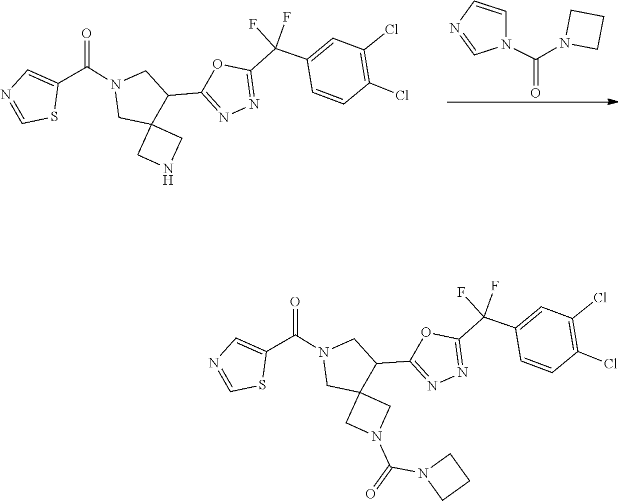 [SMILES: O=C(c1cncs1)N1CC(c2nnc(C(F)(F)c3ccc(Cl)c(Cl)c3)o2)C2(CNC2)C1 ; O=C(N1CCC1)n1ccnc1 ; O=C(c1cncs1)N1CC(c2nnc(C(F)(F)c3ccc(Cl)c(Cl)c3)o2)C2(C1)CN(C(=O)N1CCC1)C2]

### Step 1: azetidin-1-yl(1H-imidazol-1-yl)methanone

To a solution of azetidine (0.5 g, 8.76 mmol) in DMF (5 mL) and acetonitrile (1.5 mL) was added CDI (1.56 g, 9.63 mmol). The reaction was stirred at room temperature overnight then was diluted with water (50 mL) and extracted with EtOAc (75 mL×3). The combined organic layers were washed with water and brine, dried over Na₂SO₄, filtered and concentrated. The residue obtained was purified by column chromatography on silica gel (eluent: DCM/MeOH=15/1) to afford azetidin-1-yl(1H-imidazol-1-yl)methanone (500 mg, 38%) as a white solid. LCMS m/z=152.2 [M+H]⁺; ¹H NMR (400 MHz, Chloroform-d) δ 8.00 (d, J=1.2 Hz, 1H), 7.30 (s, 1H), 7.08 (s, 1H), 4.35 (t, J=8.2 Hz, 4H), 2.45 (dd, J=8.4, 7.2 Hz, 2H).

### Step 2: azetidin-1-yl(8-(5-((3,4-dichlorophenyl)difluoromethyl)-1,3,4-oxadiazol-2-yl)-6-(thiazole-5-carbonyl)-2,6-diazaspiro[3.4]octan-2-yl)methanone

To a solution of (8-(5-((3,4-dichlorophenyl)difluoromethyl)-1,3,4-oxadiazol-2-yl)-2,6-diazaspiro[3.4]octan-6-yl)(thiazol-5-yl)methanone (80.0 mg, 0.164 mmol) in DMF (3 mL) was added TEA (33.29 mg, 0.329 mmol) and azetidin-1-yl(1H-imidazol-1-yl)methanone (74.6 mg, 0.493 mmol). The reaction was heated under N₂ atmosphere at 100° C. overnight then was diluted with water (20 mL) and extracted with EtOAc (50 mL×3). The combined organic layers were washed with H₂O and brine, dried over Na₂SO₄, filtered and concentrated. The residue was purified by prep-HPLC to afford azetidin-1-yl(8-(5-((3,4-dichlorophenyl)difluoromethyl)-1,3,4-oxadiazol-2-yl)-6-(thiazole-5-carbonyl)-2,6-diazaspiro[3.4]octan-2-yl)methanone (14 mg, 15%) as a white solid. LCMS m/z=569.1 [M+H]⁺; ¹H NMR (400 MHz, Methanol-d₄) δ 9.17 (d, J=2.4 Hz, 1H), 8.37 (d, J=3.6 Hz, 1H), 7.92 (s, 1H), 7.75 (d, J=8.4 Hz, 1H), 7.64 (d, J=7.4 Hz, 1H), 4.35-3.86 (m, 13H), 2.30-2.16 (m, 2H).

### Synthesis of (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(1H-pyrazol-4-yl)methanone (I-191)

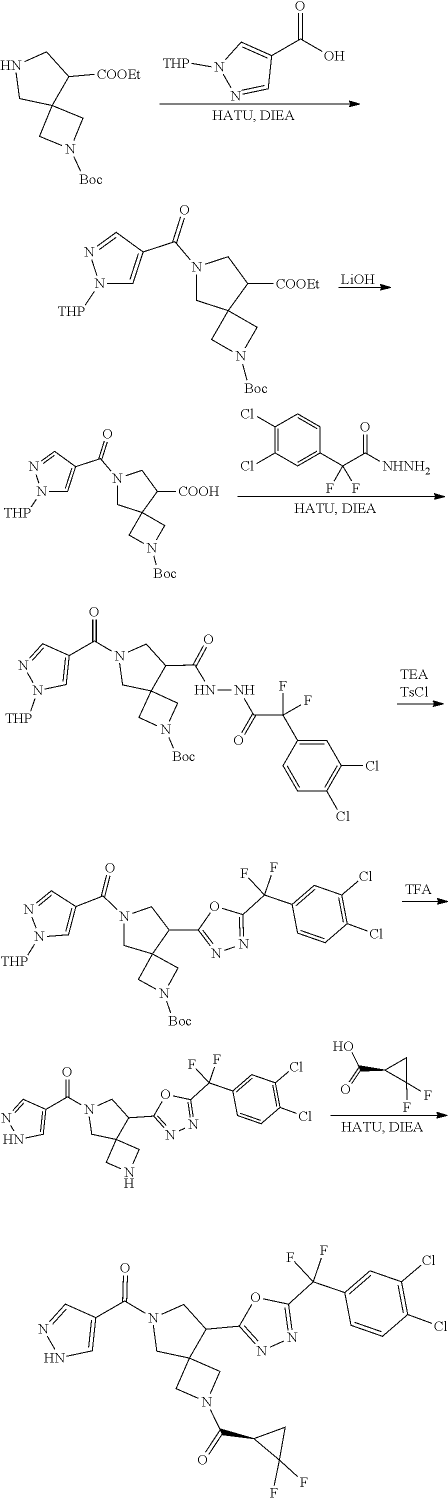

### Step 1: 2-(tert-butyl) 8-ethyl 6-(1-(tetrahydro-2H-pyran-2-yl)-1H-pyrazole-4-carbonyl)-2,6-diazaspiro[3.4]octane-2,8-dicarboxylate

To a solution of 1-(tetrahydro-2H-pyran-2-yl)-1H-pyrazole-4-carboxylic acid (502.0 mg, 2.56 mmol) in DMF (15 mL) was added HATU (973.0 mg, 2.56 mmol) and DIPEA (661.3 mg, 5.12 mmol). The mixture was stirred at room temperature for 30 min then 2-(tert-butyl) 8-ethyl 2,6-diazaspiro[3.4]octane-2,8-dicarboxylate (485.0 mg, 1.71 mmol) was added and stirring continued for another 2 h. The mixture was diluted with water (30 mL) and extracted with EtOAc (100 mL×3). The combined organic layers were washed with brine, dried over Na₂SO₄, filtered and concentrated. The mixture was purified by column chromatography on silica gel (eluent: DCM:MeOH=40:1) to afford 2-(tert-butyl) 8-ethyl 6-(1-(tetrahydro-2H-pyran-2-yl)-1H-pyrazole-4-carbonyl)-2,6-diazaspiro[3.4]octane-2,8-dicarboxylate (510.0 mg, 65%) as a white solid. LCMS m/z=463.3 [M+H]⁺; ¹H NMR (400 MHz, DMSO-d₆) δ 8.29 (d, J=13.2 Hz, 1H), 7.82 (d, J=14.4 Hz, 1H), 5.43 (d, J=10.0 Hz, 1H), 4.26-4.05 (m, 2H), 4.04-3.55 (m, 10H), 2.21-2.04 (m, 1H), 2.02-1.84 (m, 2H), 1.75-1.60 (m, 1H), 1.58-1.46 (m, 2H), 1.37 (s, 9H), 1.27-1.14 (m, 4H).

### Step 2: 2-(tert-butoxycarbonyl)-6-(1-(tetrahydro-2H-pyran-2-yl)-1H-pyrazole-4-carbonyl)-2,6-diazaspiro[3.4]octane-8-carboxylic acid

To a solution of 2-(tert-butyl) 8-ethyl 6-(1-(tetrahydro-2H-pyran-2-yl)-1H-pyrazole-4-carbonyl)-2,6-diazaspiro[3.4]octane-2,8-dicarboxylate (430 mg, 0.93 mmol) in a mixture of THF, water and EtOH (8 mL/2 mL/2 mL) was added lithium hydroxide monohydrate (118 mg, 2.79 mmol). The reaction mixture was stirred at room temperature for 4 h then diluted with water (25 mL) and extracted with ether (50 mL). The aqueous layer was collected, acidified to pH˜2 with 1M HCl and extracted with EtOAc (75 mL×3). The combined organic layers were washed with brine, dried over Na₂SO₄, filtered and concentrated to afford 2-(tert-butoxycarbonyl)-6-(1-(tetrahydro-2H-pyran-2-yl)-1H-pyrazole-4-carbonyl)-2,6-diazaspiro[3.4]octane-8-carboxylic acid (370 mg, 91%) as a yellow solid. LCMS m/z=379.1 [M−56+H]⁺.

### Step 3: tert-butyl 8-(2-(2-(3,4-dichlorophenyl)-2,2-difluoroacetyl)hydrazine-1-carbonyl)-6-(1-(tetrahydro-2H-pyran-2-yl)-1H-pyrazole-4-carbonyl)-2,6-diazaspiro[3.4]octane-2-carboxylate

To a solution of 2-(tert-butoxycarbonyl)-6-(1-(tetrahydro-2H-pyran-2-yl)-1H-pyrazole-4-carbonyl)-2,6-diazaspiro[3.4]octane-8-carboxylic acid (85.0 mg, 0.195 mmol) in DMF (2 mL) was added HATU (111.5 mg, 0.293 mmol) and DIPEA (76.0 mg, 0.586 mmol) and the mixture stirred at room temperature for 30 min. 2-(3,4-dichlorophenyl)-2,2-difluoroacetohydrazide (55 mg, 0.215 mmol) was added and stirring continued for another 2 h. The mixture was diluted with water (30 mL) and extracted with EtOAc (70 mL×3). The combined organic layers were washed with brine, dried over Na₂SO₄, filtered and concentrated. The residue obtained was purified by prep-TLC (eluent: DCM:MeOH=15:1) to afford tert-butyl 8-(2-(2-(3,4-dichlorophenyl)-2,2-difluoroacetyl)hydrazine-1-carbonyl)-6-(1-(tetrahydro-2H-pyran-2-yl)-1H-pyrazole-4-carbonyl)-2,6-diazaspiro[3.4]octane-2-carboxylate (44.0 mg, 34%) as a white solid. LCMS m/z=671.2 [M+H]⁺; ¹H NMR (400 MHz, DMSO-d₆) δ 11.11 (s, 1H), 10.39 (s, 1H), 8.38-8.21 (m, 1H), 7.95-7.73 (m, 3H), 7.62 (d, J=8.4 Hz, 1H), 5.42 (dd, J=9.8, 2.4 Hz, 1H), 4.10-3.55 (m, 11H), 3.23-3.08 (m, 1H), 2.21-2.03 (m, 1H), 2.01-1.84 (m, 2H), 1.74-1.59 (m, 1H), 1.58-1.48 (m, 2H), 1.36 (s, 9H).

### Step 4: tert-butyl 8-(5-((3,4-dichlorophenyl)difluoromethyl)-1,3,4-oxadiazol-2-yl)-6-(1-(tetrahydro-2H-pyran-2-yl)-1H-pyrazole-4-carbonyl)-2,6-diazaspiro[3.4]octane-2-carboxylate

To a solution of tert-butyl 8-(2-(2-(3,4-dichlorophenyl)-2,2-difluoroacetyl)hydrazine-1-carbonyl)-6-(1-(tetrahydro-2H-pyran-2-yl)-1H-pyrazole-4-carbonyl)-2,6-diazaspiro[3.4]octane-2-carboxylate (34.0 mg, 0.05 mmol) in DCM (2 mL) was added TsCl (29.0 mg, 0.151 mmol) and TEA (16.0 mg, 0.151 mmol). The reaction mixture was stirred at room temperature for 14 h, then was diluted with water (10 mL) and extracted with DCM (20 mL×2). The combined organic layers were washed with brine, dried over Na₂SO₄, filtered and concentrated. The mixture was purified by prep-TLC (eluent: DCM:MeOH=15:1) to afford tert-butyl 8-(5-((3,4-dichlorophenyl)difluoromethyl)-1,3,4-oxadiazol-2-yl)-6-(1-(tetrahydro-2H-pyran-2-yl)-1H-pyrazole-4-carbonyl)-2,6-diazaspiro[3.4]octane-2-carboxylate (20.0 mg, 60%) as a white solid. LCMS m/z=653.2 [M+H]⁺; ¹H NMR (400 MHz, Chloroform-d) δ 8.03 (s, 1H), 7.83 (s, 1H), 7.76 (d, J=2.2 Hz, 1H), 7.59 (d, J=8.4 Hz, 1H), 7.48 (d, J=7.6 Hz, 1H), 5.39 (dd, J=8.9, 3.4 Hz, 1H), 4.36-3.60 (m, 11H), 2.15-1.93 (m, 3H), 1.79-1.63 (m, 3H), 1.41 (s, 9H).

### Step 5: (8-(5-((3,4-dichlorophenyl)difluoromethyl)-1,3,4-oxadiazol-2-yl)-2,6-diazaspiro[3.4]octan-6-yl)(1H-pyrazol-4-yl)methanone 2,2,2-trifluoroacetate

To a solution of tert-butyl 8-(5-((3,4-dichlorophenyl)difluoromethyl)-1,3,4-oxadiazol-2-yl)-6-(1-(tetrahydro-2H-pyran-2-yl)-1H-pyrazole-4-carbonyl)-2,6-diazaspiro[3.4]octane-2-carboxylate (100 mg, 0.153 mmol) in DCM (2 mL) was added TFA (1 mL) and the reaction stirred at room temperature for 3 h. The solvent was removed under vacuum to afford (8-(5-((3,4-dichlorophenyl)difluoromethyl)-1,3,4-oxadiazol-2-yl)-2,6-diazaspiro[3.4]octan-6-yl)(1H-pyrazol-4-yl)methanone 2,2,2-trifluoroacetate (170 mg, 71%) as a yellow oil. LCMS m/z=469.1[M+H]⁺.

### Step 6: (8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(1H-pyrazol-4-yl)methanone

To a solution of (R)-2,2-difluorocyclopropane-1-carboxylic acid (18.5 mg, 0.151 mmol) in DMF (2 mL) was added HATU (87 mg, 0.226 mmol) and DIPEA (60 mg, 0.454 mmol) and the mixture stirred at room temperature for 30 min. (8-(5-((3,4-dichlorophenyl)difluoromethyl)-1,3,4-oxadiazol-2-yl)-2,6-diazaspiro[3.4]octan-6-yl)(1H-pyrazol-4-yl)methanone 2,2,2-trifluoroacetate (71 mg, 0.151 mmol) was added and stirring continued for another 2 h. The mixture was diluted with water (30 mL) and extracted with EtOAc (100 mL×3). The combined organic layers were washed with brine, dried over Na₂SO₄, filtered and concentrated. The mixture was purified by prep-TLC (eluent: DCM:MeOH=10:1) to afford 8-(5-((3,4-dichlorophenyl)difluoromethyl)-1,3,4-oxadiazol-2-yl)-2-((R)-2,2-difluorocyclopropane-1-carbonyl)-2,6-diazaspiro[3.4]octan-6-yl)(1H-pyrazol-4-yl)methanone (25 mg, 29%) as a white solid. LCMS m/z=573.1 [M+H]⁺; ¹H NMR (400 MHz, Methanol-d₄) δ 8.29-7.82 (m, 3H), 7.73 (d, J=8.4 Hz, 1H), 7.66-7.53 (m, 1H), 4.62-3.88 (m, 9H), 2.66-2.42 (m, 1H), 2.04-1.93 (m, 1H), 1.85-1.67 (m, 1H).

Table 27: The compounds listed in Table 27 were synthesized from (8-(5-((3,4-dichlorophenyl)difluoromethyl)-1,3,4-oxadiazol-2-yl)-2,6-diazaspiro[3.4]octan-6-yl)(1H-pyrazol-4-yl)methanone according to the procedures outlined for I-191 using the appropriate commercially available reagents and/or intermediates described elsewhere.

TABLE 27

| Example | | | |
|---|---|---|---|
| Number | Compound | ¹HNMR | LCMS |
| I-177 | 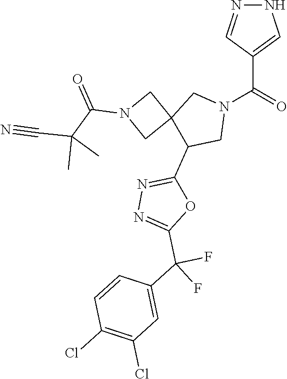 [SMILES: CC(C)(C#N)C(=O)N1CC2(CN(C(=O)c3cn[nH]c3)CC2c2nnc(C(F)(F)c3ccc(Cl)c(Cl)c3)o2)C1] | ¹H NMR (400 MHz, Methanol-d₄) δ 8.16 (s, 1H), 7.99 (s, 1H), 7.91- 7.84 (m, 1H), 7.76-7.67 (m, 1H), 7.66-7.57 (m, 1H), 4.83-4.51 (m, 2H), 4.36-3.94 (m, 7H), 1.62- 1.41 (m, 6H). | m/z = 564.1 [M + H]⁺ |

### Synthesis of (8-(5-((3,4-dichlorophenyl)difluoromethyl)-1,3,4-oxadiazol-2-yl)-2-((tetrahydrofuran-2-yl)methyl)-2,6-diazaspiro[3.4]octan-6-yl)(1H-pyrazol-5-yl)methanone formate (I-139

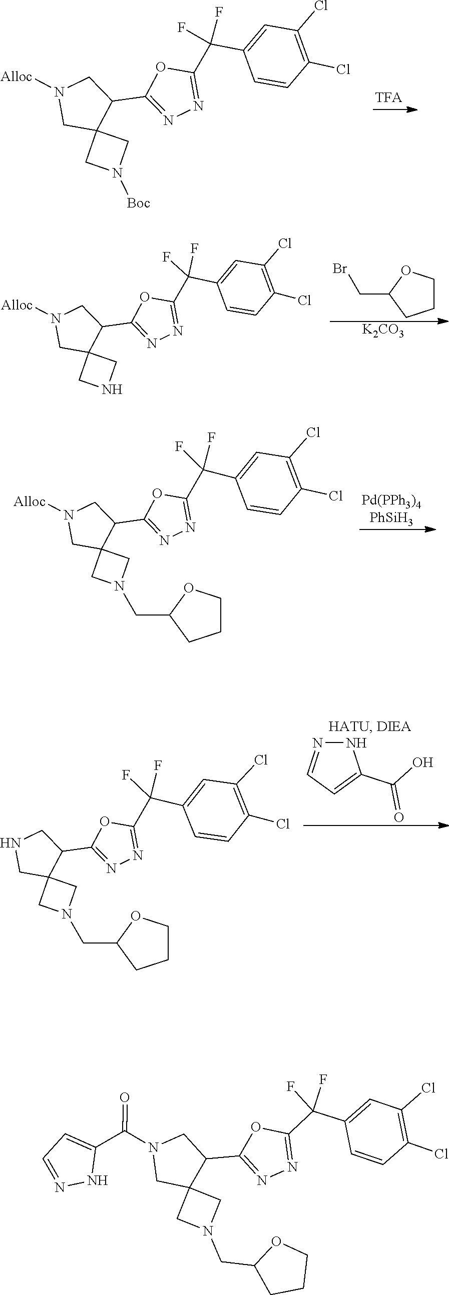

### Step 1: allyl 8-(5-((3,4-dichlorophenyl)difluoromethyl)-1,3,4-oxadiazol-2-yl)-2,6-diazaspiro[3.4]octane-6-carboxylate

To a solution of 6-allyl 2-(tert-butyl) 8-(5-((3,4-dichlorophenyl)difluoromethyl)-1,3,4-oxadiazol-2-yl)-2,6-diazaspiro[3.4]octane-2,6-dicarboxylate (5.8 g, 10.3 mmol) in DCM (50 mL) was added TFA (25 mL) and the reaction stirred for 2 h. The solvent was removed under vacuum to afford allyl 8-(5-((3,4-dichlorophenyl)difluoromethyl)-1,3,4-oxadiazol-2-yl)-2,6-diazaspiro[3.4]octane-6-carboxylate (5.2 g, quant.) as a yellow semi-solid. LCMS m/z=459.0 [M+H]⁺.

### Step 2: allyl 8-(5-((3,4-dichlorophenyl)difluoromethyl)-1,3,4-oxadiazol-2-yl)-2-((tetrahydrofuran-2-yl)methyl)-2,6-diazaspiro[3.4]octane-6-carboxylate

To a solution of allyl 8-(5-((3,4-dichlorophenyl)difluoromethyl)-1,3,4-oxadiazol-2-yl)-2,6-diazaspiro[3.4]octane-6-carboxylate (1.0 g, 2.2 mmol) in ACN (10 mL) was added K₂CO₃ (904 mg, 6.5 mmol) and KI (72 mg, 6.5 mmol). The reaction mixture was heated in a sealed tube at 80° C. overnight then was filtrated through celite and the filtrate concentrated. The residue was purified by column chromatography on silica gel (eluent: EtOAc) to afford allyl 8-(5-((3,4-dichlorophenyl)difluoromethyl)-1,3,4-oxadiazol-2-yl)-2-((tetrahydrofuran-2-yl)methyl)-2,6-diazaspiro[3.4]octane-6-carboxylate (600 mg, 51%) as a brown oil. LCMS m/z=543.1 [M+H]⁺; ¹H NMR (400 MHz, Chloroform-d) δ 7.77 (s, 1H), 7.60 (d, J=8.4 Hz, 1H), 7.51 (d, J=8.5 Hz, 1H), 6.00-5.85 (m, 1H), 5.30 (d, J=17.7 Hz, 1H), 5.22 (d, J=10.4 Hz, 1H), 4.59 (d, J=5.6 Hz, 2H), 3.93-3.56 (m, 8H), 3.56-3.00 (m, 4H), 2.43 (s, 2H), 1.81 (s, 3H).

### Step 3: 2-((3,4-dichlorophenyl)difluoromethyl)-5-(2-((tetrahydrofuran-2-yl)methyl)-2,6-diazaspiro[3.4]octan-8-yl)-1,3,4-oxadiazole

To a solution of allyl 8-(5-((3,4-dichlorophenyl)difluoromethyl)-1,3,4-oxadiazol-2-yl)-2-((tetrahydrofuran-2-yl)methyl)-2,6-diazaspiro[3.4]octane-6-carboxylate (800 mg, 1.47 mmol) in THE (20 mL) was added Pd(PPh₃)₄ (170 mg, 0.15 mmol) and phenylsilane (796 mg, 7.4 mmol). The reaction was stirred at room temperature for 4 h then was diluted with water (20 mL) and extracted with EtOAc (80 mL×2). The combined organic layers were washed with brine, dried over Na₂SO₄, filtered and concentrated. The residue was purified by column chromatography on silica gel (eluent: DCM:MeOH=10:1) to afford 2-((3,4-dichlorophenyl)difluoromethyl)-5-(2-((tetrahydrofuran-2-yl)methyl)-2,6-diazaspiro[3.4]octan-8-yl)-1,3,4-oxadiazole (550 mg, 50%) as a black oil. LCMS m/z=459.0 [M+H]⁺; ¹H NMR (400 MHz, Chloroform-d) δ 7.77 (s, 1H), 7.60 (d, J=8.3 Hz, 1H), 7.51 (d, J=8.4 Hz, 1H), 3.83-3.75 (m, 2H), 3.73-3.67 (m, 1H), 3.63-3.58 (m, 1H), 3.40-3.28 (m, 5H), 3.21 (t, J=8.0 Hz, 1H), 3.09-3.00 (m, 1H), 2.47 (d, J=5.6 Hz, 2H), 2.34 (t, J=6.6 Hz, 1H), 1.87-1.78 (m, 4H).

### Step 4: (8-(5-((3,4-dichlorophenyl)difluoromethyl)-1,3,4-oxadiazol-2-yl)-2-((tetrahydrofuran-2-yl)methyl)-2,6-diazaspiro[3.4]octan-6-yl)(1H-pyrazol-5-yl)methanone formate

To a solution of 1H-pyrazole-5-carboxylic acid (23.44 mg, 0.206 mmol) in DMF (2 mL) was added HATU (98.1 mg, 0.258 mmol) and DIPEA (67.0 mg, 0.516 mmol) and the mixture stirred at room temperature for 30 min. 2-((3,4-dichlorophenyl)difluoromethyl)-5-(2-((tetrahydrofuran-2-yl)methyl)-2,6-diazaspiro[3.4]octan-8-yl)-1,3,4-oxadiazole (80 mg, 0.172 mmol) was added and stirring continued for another 2 h. The mixture was diluted with water (10 mL) and extracted with EtOAc (30 mL×3). The combined organic layers were washed with brine, dried over Na₂SO₄, filtered and concentrated. The residue obtained was purified by prep-HPLC to afford (8-(5-((3,4-dichlorophenyl)difluoromethyl)-1,3,4-oxadiazol-2-yl)-2-((tetrahydrofuran-2-yl)methyl)-2,6-diazaspiro[3.4]octan-6-yl)(1H-pyrazol-5-yl)methanone (38 mg, 40%) as a white solid. LCMS m/z=553.1 [M+H]⁺; ¹H NMR (400 MHz, Methanol-d₄) δ 8.32 (s, 1H), 7.88 (dd, J=10.2, 2.0 Hz, 1H), 7.77-7.66 (m, 2H), 7.66-7.57 (m, 1H), 6.78 (dd, J=9.2, 2.4 Hz, 1H), 4.61-4.48 (m, 1H), 4.42-4.33 (m, 1H), 4.28-3.95 (m, 8H), 3.92-3.80 (m, 1H), 3.80-3.67 (m, 1H), 3.23-2.95 (m, 2H), 2.11-1.99 (m, 1H), 1.98-1.84 (m, 2H), 1.64-1.48 (m, 1H).

Table 28: The compounds listed in Table 28 were synthesized from 2-((3,4-dichlorophenyl)difluoromethyl)-5-(2-((tetrahydrofuran-2-yl)methyl)-2,6-diazaspiro[3.4]octan-8-yl)-1,3,4-oxadiazole according to the procedures outlined for I-139 using the appropriate commercially available reagents and/or intermediates described elsewhere.

TABLE 28

| Example | | | |
|---|---|---|---|
| Number | Compound | ¹HNMR | LCMS |
| I-136 | 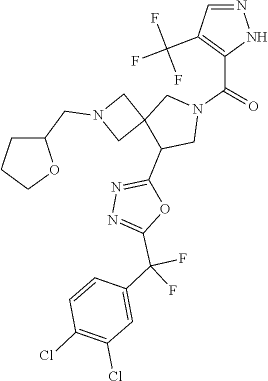 [SMILES: O=C(c1[nH]ncc1C(F)(F)F)N1CC(c2nnc(C(F)(F)c3ccc(Cl)c(Cl)c3)o2)C2(CN(CC3CCCO3)C2)C1] | ¹H NMR (400 MHz, CDCl₃) δ 7.83-7.78 (m, 1H), 7.78-7.71 (m, 1H), 7.62-7.55 (m, 1H), 7.53- 7.46 (m, 1H), 4.22- 3.11 (m, 13H), 2.61- 2.39 (m, 2H), 1.98-1.77 (m, 3H), 1.47-1.35 (m, 1H). | m/z = 621.1 [M + H]⁺ |
| I-142 | 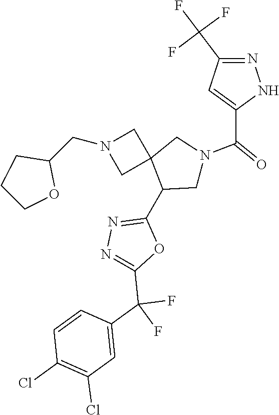 [SMILES: O=C(c1cc(C(F)(F)F)n[nH]1)N1CC(c2nnc(C(F)(F)c3ccc(Cl)c(Cl)c3)o2)C2(CN(CC3CCCO3)C2)C1] | ¹H NMR (400 MHz, CD₃OD) δ 7.87 (d, J = 6.4 Hz, 1H), 7.76-7.58 (m, 2H), 7.09 (d, J = 12.2 Hz, 1H), 4.52-4.00 (m, 10H), 3.88 (s, 1H), 3.81- 3.70 (m, 1H), 3.43- 3.32 (m, 1H), 3.26-3.13 (m, 1H), 2.15-2.04 (m, 1H), 1.99-1.86 (m, 2H), 1.65-1.51 (m, 1H), 1.39- 1.19 (m, 1H). | m/z = 621.1 [M + H]⁺ |
| I-143 | 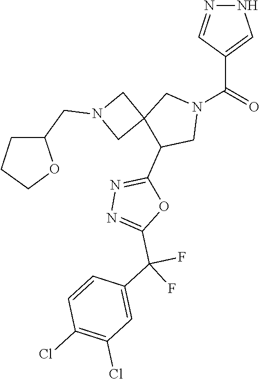 [SMILES: O=C(c1cn[nH]c1)N1CC(c2nnc(C(F)(F)c3ccc(Cl)c(Cl)c3)o2)C2(CN(CC3CCCO3)C2)C1] | ¹H NMR (400 MHz, Methanol-d₄) δ 8.41 (s, 1H), 8.05 (d, J = 10.4 Hz, 2H), 7.89 (d, J = 7.0 Hz, 1H), 7.74 (d, J = 8.4 Hz, 1H), 7.63 (t, J = 7.4 Hz, 1H), 4.30-3.65 (m, 12H), 3.01-2.71 (m, 2H), 2.04-1.78 (m, 3H), 1.59-1.40 (m, 1H). | m/z = 553.0 [M + H]⁺ |
| I-141 | 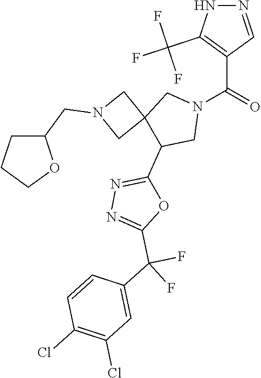 [SMILES: O=C(c1cn[nH]c1C(F)(F)F)N1CC(c2nnc(C(F)(F)c3ccc(Cl)c(Cl)c3)o2)C2(CN(CC3CCCO3)C2)C1] | ¹H NMR (400 MHz, CD₃OD) δ 8.11 (d, J = 9.0 Hz, 1H), 7.89 (d, J = 13.6 Hz, 1H), 7.78-7.69 (m, 1H), 7.68-7.59 (m, 1H), 4.46-3.70 (m, 12H), 3.27-2.82 (m, 2H), 2.14-1.99 (m, 1H), 1.92 (s, 2H), 1.66-1.45 (m, 1H). | m/z = 621.1 [M + H]⁺ |
| I-135 | 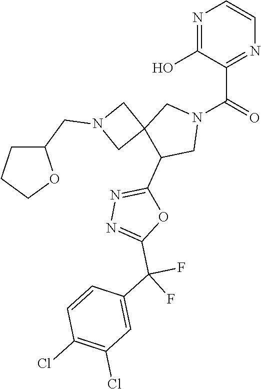 [SMILES: O=C(c1nccnc1O)N1CC(c2nnc(C(F)(F)c3ccc(Cl)c(Cl)c3)o2)C2(CN(CC3CCCO3)C2)C1] | ¹H NMR (400 MHz, CD₃OD) δ 7.91 (dd, J = 7.3, 2.1 Hz, 1H), 7.79- 7.73 (m, 1H), 7.71-7.49 (m, 3H), 4.62-3.74 (m, 13H), 3.43 (t, J = 14.9 Hz, 1H), 3.29-3.19 (m, 1H), 2.18-2.06 (m, 1H), 2.00-1.86 (m, 2H), 1.65- 1.53 (m, 1H). | m/z = 581.1 [M + H]⁺ |
| I-140 | 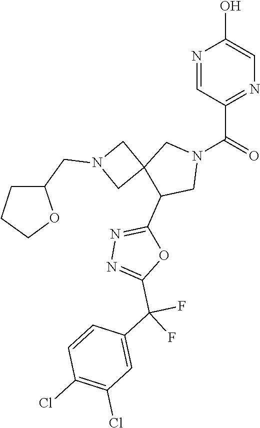 [SMILES: O=C(c1cnc(O)cn1)N1CC(c2nnc(C(F)(F)c3ccc(Cl)c(Cl)c3)o2)C2(CN(CC3CCCO3)C2)C1] | ¹H NMR (400 MHz, CD₃OD) δ 8.32 (d, J = 7.0 Hz, 1H), 8.08-7.98 (m, 2H), 7.89 (d, J = 8.3 Hz, 1H), 7.74 (d, J = 8.4 Hz, 1H), 7.69-7.60 (m, 1H), 4.55-3.65 (m, 11H), 3.20-2.77 (m, 3H), 2.08-1.81 (m, 3H), 1.60-1.45 (m, 1H). | m/z = 581.1 [M + H]⁺ |

### Synthesis of 1-(1-(3-(2-((S)-2,2-dimethylcyclopropane-1-carbonyl)-6-(thiazole-5-carbonyl)-2,6-diazaspiro[3.4]octan-8-yl)-1,2,4-oxadiazol-5-yl)ethyl)-3-methyl-1H-pyrazole-5-carboxylic acid (I-188)

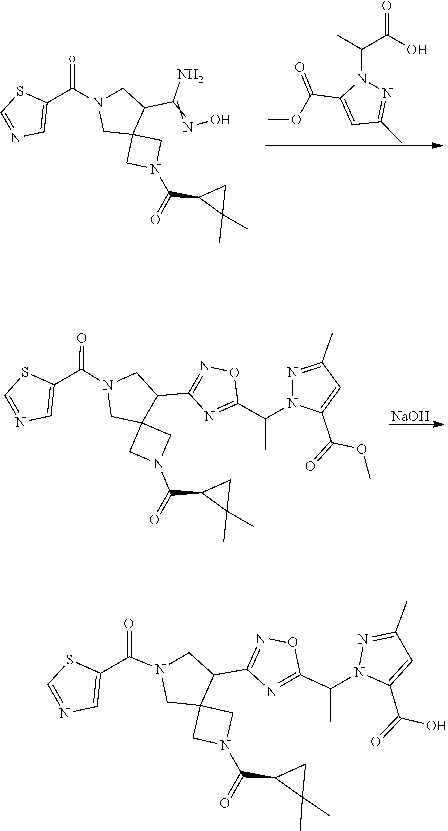

### Step 1: methyl 1-(1-(3-(2-((S)-2,2-dimethylcyclopropane-1-carbonyl)-6-(thiazole-5-carbonyl)-2,6-diazaspiro[3.4]octan-8-yl)-1,2,4-oxadiazol-5-yl)ethyl)-3-methyl-1H-pyrazole-5-carboxylate

To a solution of 2-(5-(methoxycarbonyl)-3-methyl-1H-pyrazol-1-yl)propanoic acid (200 mg, 3.6 mmol) in DMF (5.0 mL) was added 2-((S)-2,2-dimethylcyclopropane-1-carbonyl)-N′-hydroxy-6-(thiazole-5-carbonyl)-2,6-diazaspiro[3.4]octane-8-carboximidamide (377 mg, 1.0 mmol) and EDCI (360 mg, 1.89 mmol). The resulting mixture was heated at 60° C. for 3 h and then warmed to 110° C. and heating continued overnight. The mixture was diluted with water (30 mL) and extracted with EtOAc (50 mL×2). The combined organic layers were washed with brine, dried over Na₂SO₄, filtered and concentrated. The residue was purified by RP-column to afford methyl 1-(1-(3-(2-((S)-2,2-dimethylcyclopropane-1-carbonyl)-6-(thiazole-5-carbonyl)-2,6-diazaspiro [3.4]octan-8-yl)-1,2,4-oxadiazol-5-yl)ethyl)-3-methyl-1H-pyrazole-5-carboxylate (124 mg, 24%) as a white solid. LCMS m/z=554.2 [M+H]⁺; ¹H NMR (400 MHz, CD₃OD) δ 9.17 (s, 1H), 8.36 (q, J=7.4, 6.5 Hz, 1H), 6.59 (d, J=6.5 Hz, 1H), 6.09-6.00 (m, 1H), 4.59-3.81 (m, 11H), 3.76-3.69 (m, 1H), 2.44-2.35 (m, 3H), 2.05-1.96 (m, 3H), 1.22-1.00 (m, 8H), 0.83-0.70 (m, 1H).

### Step 2: 1-(1-(3-(2-((S)-2,2-dimethylcyclopropane-1-carbonyl)-6-(thiazole-5-carbonyl)-2,6-diazaspiro[3.4]octan-8-yl)-1,2,4-oxadiazol-5-yl)ethyl)-3-methyl-1H-pyrazole-5-carboxylic acid

To a solution of methyl 1-(1-(3-(2-((S)-2,2-dimethylcyclopropane-1-carbonyl)-6-(thiazole-5-carbonyl)-2,6-diazaspiro[3.4]octan-8-yl)-1,2,4-oxadiazol-5-yl)ethyl)-3-methyl-1H-pyrazole-5-carboxylate (130 mg, 0.23 mmol) in MeOH (1.0 mL) was added NaOH (10% w/v in water, 1 mL). The mixture was stirred at room temperature for 2 h then was diluted with water (20 mL), acidified with 1 M HCl to pH=3 and extracted with EtOAc (50 mL×3). The combined organic layers were washed with brine, dried over Na₂SO₄, filtered and concentrated to afford 1-(1-(3-(2-((S)-2,2-dimethylcyclopropane-1-carbonyl)-6-(thiazole-5-carbonyl)-2,6-diazaspiro[3.4]octan-8-yl)-1,2,4-oxadiazol-5-yl)ethyl)-3-methyl-1H-pyrazole-5-carboxylic acid (124 mg, 97%) as a white solid. LCMS m/z=554.2 [M+H]⁺; ¹H NMR (400 MHz, CD₃OD) δ 9.17 (s, 1H), 8.36 (q, J=7.4, 6.5 Hz, 1H), 6.59 (d, J=6.5 Hz, 1H), 6.09-6.00 (m, 1H), 4.59-3.81 (m, 8H), 3.76-3.69 (m, 1H), 2.44-2.35 (m, 3H), 2.05-1.96 (m, 3H), 1.22-1.00 (m, 8H), 0.83-0.70 (m, 1H).

### Synthesis of (2-((R)-2,2-difluorocyclopropane-1-carbonyl)-8-(6-phenoxybenzo[d]oxazol-2-yl)-2,6-diazaspiro[3.4]octan-6-yl)(thiazol-5-yl)methanone (I-144)

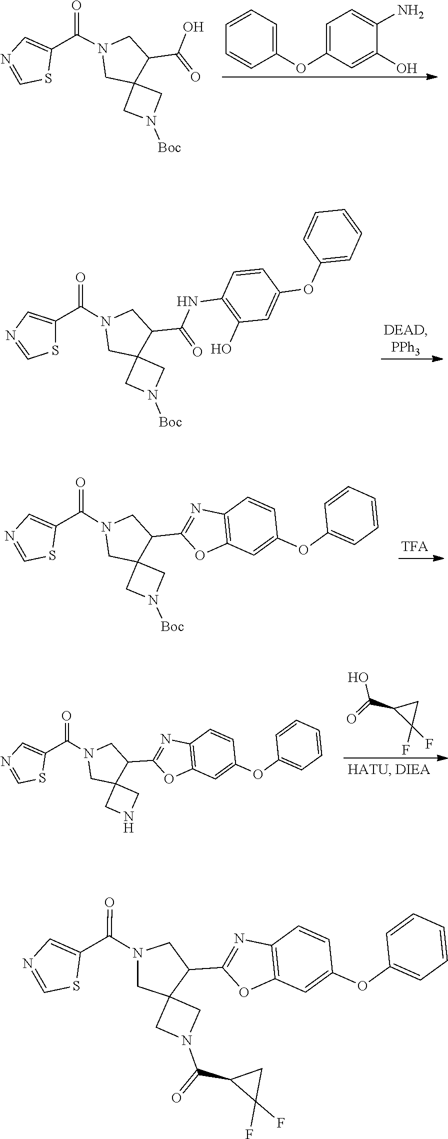

### Step 1: tert-butyl 8-((2-hydroxy-4-phenoxyphenyl)carbamoyl)-6-(thiazole-5-carbonyl)-2,6-diazaspiro[3.4]octane-2-carboxylate

To a solution of 2-(tert-butoxycarbonyl)-6-(thiazole-5-carbonyl)-2,6-diazaspiro[3.4]octane-8-carboxylic acid (0.5 g, 1.36 mmol) in DMF (5 mL) under a N₂ atmosphere was added 2-amino-5-phenoxyphenol (302 mg, 1.50 mmol) and TEA (688 mg, 6.80 mmol). The reaction was cooled to −20° C. and T₃P (50% solution in DMF) (1.3 g, 2.04 mmol) was added. The reaction mixture was heated at 110° C. overnight then was diluted with water (20 mL) and extracted with EtOAc (30 mL×3). The combined organic layers were washed with brine, dried over Na₂SO₄, filtered and concentrated. The mixture was purified by RP-column and prep-TLC (eluent: Pet. DCM:MeOH=15:1) to afford tert-butyl 8-((2-hydroxy-4-phenoxyphenyl)carbamoyl)-6-(thiazole-5-carbonyl)-2,6-diazaspiro[3.4]octane-2-carboxylate (120 mg, 16%) as a brown oil. LCMS m/z=551.2 [M+H]⁺; ¹H NMR (400 MHz, CD₃OD) δ 9.15 (s, 1H), 8.38 (d, J=8.4 Hz, 1H), 7.55 (t, J=9.3 Hz, 1H), 7.34 (t, J=7.8 Hz, 2H), 7.09 (t, J=7.4 Hz, 1H), 6.98 (d, J=8.0 Hz, 2H), 6.57-6.50 (m, 1H), 6.45 (d, J=8.6 Hz, 1H), 4.31-3.83 (m, 8H), 3.56-3.45 (m, 1H), 1.42 (d, J=3.2 Hz, 9H).

### Step 2: tert-butyl 8-(6-phenoxybenzo[d]oxazol-2-yl)-6-(thiazole-5-carbonyl)-2,6-diazaspiro[3.4]octane-2-carboxylate

To a solution of tert-butyl 8-((2-hydroxy-4-phenoxyphenyl)carbamoyl)-6-(thiazole-5-carbonyl)-2,6-diazaspiro[3.4]octane-2-carboxylate (60 mg, 0.11 mmol) in toluene (1.5 mL) under a N₂ atmosphere was added PPh₃ (58 mg, 0.22 mmol) and DEAD (38 mg, 0.22 mmol). The reaction mixture was heated at 120° C. for 2 h then was diluted with water (10 mL) and extracted with EtOAc (20 mL×3). The combined organic layers were washed with brine, dried over Na₂SO₄, filtered and concentrated. The mixture was purified by prep-TLC (eluent: Pet. DCM:MeOH=15:1) to afford tert-butyl 8-(6-phenoxybenzo[d]oxazol-2-yl)-6-(thiazole-5-carbonyl)-2,6-diazaspiro[3.4]octane-2-carboxylate (50 mg, 86%) as a colorless oil. LCMS m/z=533.2 [M+H]⁺; ¹H NMR (400 MHz, CD₃OD) δ 9.16 (s, 1H), 8.41 (s, 1H), 7.69-7.61 (m, 3H), 7.60-7.52 (m, 1H), 7.37 (t, J=8.0 Hz, 2H), 7.25-6.95 (m, 5H), 4.41-4.19 (m, 3H), 4.11-3.87 (m, 6H), 3.59-3.52 (m, 1H), 1.44-1.31 (m, 9H).

### Step 3: (8-(6-phenoxybenzo[d]oxazol-2-yl)-2,6-diazaspiro[3.4]octan-6-yl)(thiazol-5-yl) methanone

To a solution of tert-butyl 8-(6-phenoxybenzo[d]oxazol-2-yl)-6-(thiazole-5-carbonyl)-2,6-diazaspiro[3.4]octane-2-carboxylate (35 mg, 0.066 mmol) in DCM (1.5 mL) was added TFA (1 mL) and the reaction stirred at room temperature for 2 h. The solvent was removed under vacuum to afford (8-(6-phenoxybenzo[d]oxazol-2-yl)-2,6-diazaspiro[3.4]octan-6-yl)(thiazol-5-yl)methanone (28 mg, quant.) which was used directly in the next step. LCMS m/z=432.8 [M+H]⁺.

### Step 4: (2-((R)-2,2-difluorocyclopropane-1-carbonyl)-8-(6-phenoxybenzo[d]oxazol-2-yl)-2,6-diazaspiro[3.4]octan-6-yl)(thiazol-5-yl)methanone

To a solution of (R)-2,2-difluorocyclopropane-1-carboxylic acid (14 mg, 0.11 mmol) in DMF (1.0 mL) was added HATU (53 mg, 0.14 mmol) and the mixture stirred at room temperature for 30 min. (8-(6-phenoxybenzo[d]oxazol-2-yl)-2,6-diazaspiro[3.4]octan-6-yl)(thiazol-5-yl)methanone (40 mg, 0.09 mmol) and DIPEA (48 mg, 0.36 mmol) were added and the reaction stirred for another 3 h. The mixture was diluted with water (10 mL) and extracted with EtOAc (30 mL×2). The combined organic layers were washed with brine, dried over Na₂SO₄, filtered and concentrated. The residue obtained was purified by prep-TLC (eluent: Pet. DCM:MeOH=15:1) to afford (2-((R)-2,2-difluorocyclopropane-1-carbonyl)-8-(6-phenoxybenzo[d]oxazol-2-yl)-2,6-diazaspiro[3.4]octan-6-yl)(thiazol-5-yl)methanone (32 mg, 67%) as a white solid. LCMS m/z=537.1 [M+H]⁺; ¹H NMR (400 MHz, CD₃OD) δ 9.16 (s, 1H), 8.41 (s, 1H), 7.72-7.56 (m, 1H), 7.46-7.31 (m, 2H), 7.24-6.95 (m, 5H), 4.68-3.92 (m, 9H), 2.66-2.40 (m, 1H), 2.03-1.90 (m, 1H), 1.83-1.62 (m, 1H).

### Synthesis of (2-((R)-2,2-difluorocyclopropane-1-carbonyl)-8-(5-phenoxybenzo[d]oxazol-2-yl)-2,6-diazaspiro[3.4]octan-6-yl)(thiazol-5-yl)methanone (I-145)

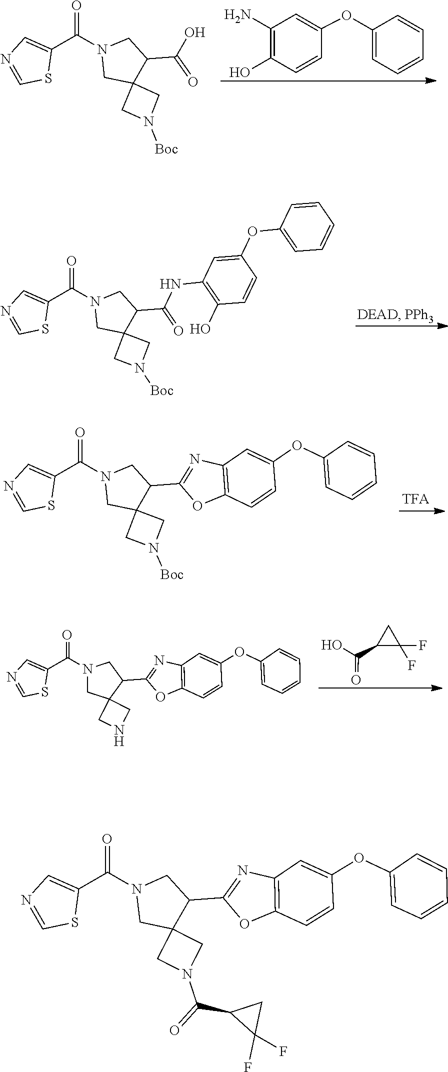

(2-((R)-2,2-difluorocyclopropane-1-carbonyl)-8-(5-phenoxybenzo[d]oxazol-2-yl)-2,6-diazaspiro[3.4]octan-6-yl)(thiazol-5-yl)methanone was synthesized from 2-(tert-butoxycarbonyl)-6-(thiazole-5-carbonyl)-2,6-diazaspiro[3.4]octane-8-carboxylic acid according to the procedures outlined for I-144 using the appropriate commercially available reagents and/or intermediates described elsewhere. LCMS m/z=537.0 [M+H]⁺; ¹H NMR (400 MHz, CD₃OD) δ 9.17 (s, 1H), 8.41 (s, 1H), 7.58 (q, J=7.1, 6.5 Hz, 1H), 7.38-7.21 (m, 3H), 7.13-7.04 (m, 2H), 6.96 (d, J=8.0 Hz, 2H), 4.68-3.95 (m, 9H), 2.67-2.38 (m, 1H), 2.01-1.63 (m, 2H).

### Synthesis of (8-(5-(1-(3-cyclopentyl-1H-pyrazol-1-yl)ethyl)-1,2,4-oxadiazol-3-yl)-2-((R)-2,2-difluorocyclopropane-1-carbonyl)-2,6-diazaspiro[3.4]octan-6-yl)(5-hydroxypyrazin-2-yl)methanone (I-134)

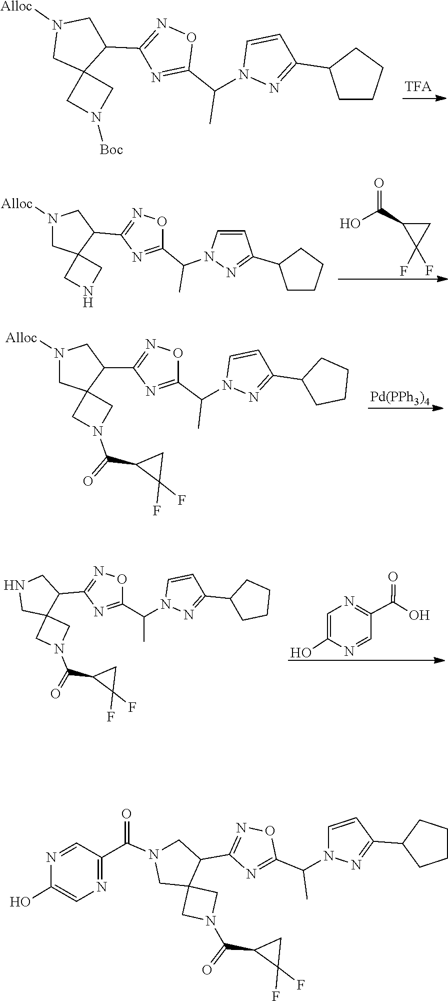

### Step 1: allyl 8-(5-(1-(3-cyclopentyl-1H-pyrazol-1-yl)ethyl)-1,2,4-oxadiazol-3-yl)-2,6-diazaspiro[3.4]octane-6-carboxylate

To a solution of 6-allyl 2-(tert-butyl) 8-(5-(1-(3-cyclopentyl-1H-pyrazol-1-yl)ethyl)-1,2,4-oxadiazol-3-yl)-2,6-diazaspiro[3.4]octane-2,6-dicarboxylate (910 mg, 1.73 mmol) in DCM (10 mL) was added TFA (5 mL) and the reaction stirred at room temperature for 2 h. The solvent was removed under vacuum to afford allyl 8-(5-(1-(3-cyclopentyl-1H-pyrazol-1-yl)ethyl)-1,2,4-oxadiazol-3-yl)-2,6-diazaspiro[3.4]octane-6-carboxylate (1.5 g) as a yellow oil. LCMS m/z=427.20 [M+H]⁺.

### Step 2: allyl 8-(5-(1-(3-cyclopentyl-1H-pyrazol-1-yl)ethyl)-1,2,4-oxadiazol-3-yl)-2-((R)-2,2-difluorocyclopropane-1-carbonyl)-2,6-diazaspiro[3.4]octane-6-carboxylate

To a solution of allyl 8-(5-(1-(3-cyclopentyl-1H-pyrazol-1-yl)ethyl)-1,2,4-oxadiazol-3-yl)-2,6-diazaspiro[3.4]octane-6-carboxylate (737 mg, 1.73 mmol) in DCM (7 mL) was added HATU (722 mg, 1.9 mmol) and DIPEA (1.8 g, 13.8 mmol) and the mixture stirred at room temperature for 30 min. (R)-2,2-difluorocyclopropane-1-carboxylic acid (232 mg, 1.9 mmol) was added and stirring continued overnight. The mixture was diluted with water (30 mL) and extracted with DCM (50 mL×3). The combined organic layers were washed with water and brine, dried over Na₂SO₄, filtered and concentrated. The residue obtained was purified by RP-column (C18, 40 g, 53% ACN in water) and then by column chromatography on silica gel (eluent: DCM:MeOH=50:1) to afford allyl 8-(5-(1-(3-cyclopentyl-1H-pyrazol-1-yl)ethyl)-1,2,4-oxadiazol-3-yl)-2-((R)-2,2-difluorocyclopropane-1-carbonyl)-2,6-diazaspiro[3.4]octane-6-carboxylate (570 mg, 62%) as colourless oil. LCMS m/z=531.3 [M+H]⁺; ¹H NMR (400 MHz, Chloroform-d) δ 7.46 (s, 1H), 6.14 (s, 1H), 6.02-5.87 (m, 1H), 5.84-5.64 (m, 1H), 5.36-5.19 (m, 3H), 4.61 (d, J=5.5 Hz, 2H), 4.40 (d, J=24.6 Hz, 1H), 4.31-4.15 (m, 1H), 4.12-3.98 (m, 2H), 3.92-3.79 (m, 4H), 3.77-3.69 (m, 1H), 3.66-3.57 (m, 1H), 3.18-3.01 (m, 1H), 2.07-1.89 (m, 5H), 1.78-1.54 (m, 8H).

### Step 3: (8-(5-(1-(3-cyclopentyl-1H-pyrazol-1-yl)ethyl)-1,2,4-oxadiazol-3-yl)-2,6-diazaspiro[3.4]octan-2-yl)((R)-2,2-difluorocyclopropyl)methanone

To a solution of allyl 8-(5-(1-(3-cyclopentyl-1H-pyrazol-1-yl)ethyl)-1,2,4-oxadiazol-3-yl)-2-((R)-2,2-difluorocyclopropane-1-carbonyl)-2,6-diazaspiro[3.4]octane-6-carboxylate (427 mg, 0.80 mmol) and Pd(PPh₃)₄ (93 mg, 0.08 mmol) in THF (5 mL) was added PhSiH₃ (435 mg, 4.0 mmol). The mixture was stirred at room temperature for 1 h then the solvent was removed and the residue obtained purified by reverse column (C18, 80 g, 35% ACN in water) to afford (8-(5-(1-(3-cyclopentyl-1H-pyrazol-1-yl)ethyl)-1,2,4-oxadiazol-3-yl)-2,6-diazaspiro[3.4]octan-2-yl)((R)-2,2-difluorocyclopropyl)methanone (317 mg, 72%) as a yellow oil. LCMS m/z=447.2 [M+H]⁺; ¹H NMR (400 MHz, Chloroform-d) δ 7.49-7.42 (m, 2H), 6.14 (d, J=2.6 Hz, 1H), 5.73 (d, J=8.2 Hz, 1H), 4.36-3.71 (m, 5H), 3.48 (s, 1H), 3.36-3.20 (m, 4H), 3.11-2.99 (m, 1H), 2.03-1.92 (m, 5H), 1.81-1.54 (m, 8H).

### Step 4: (8-(5-(1-(3-cyclopentyl-1H-pyrazol-1-yl)ethyl)-1,2,4-oxadiazol-3-yl)-2-((R)-2,2-difluorocyclopropane-1-carbonyl)-2,6-diazaspiro[3.4]octan-6-yl)(5-hydroxypyrazin-2-yl)methanone

To a solution of 5-hydroxypyrazine-2-carboxylic acid (90 mg, 0.64 mmol) in DCM (5 mL) was added HATU (245 mg, 0.64 mmol) and DIPEA (208 g, 1.6 mmol) and the mixture stirred at room temperature for 10 min. (8-(5-(1-(3-cyclopentyl-1H-pyrazol-1-yl)ethyl)-1,2,4-oxadiazol-3-yl)-2,6-diazaspiro[3.4]octan-2-yl)((R)-2,2-difluorocyclopropyl)methanone (240 mg, 0.54 mmol) was added and stirring continued for another 2 h. The mixture was diluted with water (20 mL) and extracted with DCM (20 mL×3). The combined organic layers were washed with water and brine, dried over Na₂SO₄, filtered and concentrated. The residue obtained was purified by prep-HPLC to afford (8-(5-(1-(3-cyclopentyl-1H-pyrazol-1-yl)ethyl)-1,2,4-oxadiazol-3-yl)-2-((R)-2,2-difluorocyclopropane-1-carbonyl)-2,6-diazaspiro[3.4]octan-6-yl)(5-hydroxypyrazin-2-yl)methanone (116 mg, 38%) as white solid. LCMS m/z=569.2 [M+H]⁺; ¹H NMR (400 MHz, DMSO-d₆) δ 7.95 (s, 2H), 7.82-7.73 (m, 1H), 6.13 (s, 1H), 5.98 (q, J=7.4 Hz, 1H), 4.49-3.69 (m, 9H), 3.01-2.86 (m, 1H), 2.68-2.56 (m, 1H), 2.01-1.76 (m, 7H), 1.65 (s, 2H), 1.56 (d, J=11.8 Hz, 4H).

Table 29: The compounds listed in Table 29 were synthesized from (8-(5-(1-(3-cyclopentyl-1H-pyrazol-1-yl)ethyl)-1,2,4-oxadiazol-3-yl)-2,6-diazaspiro[3.4]octan-2-yl)((R)-2,2-difluorocyclopropyl)methanone according to the procedures outlined for I-134 using the appropriate commercially available reagents and/or intermediates described elsewhere.

TABLE 29

| Example | | | |
|---|---|---|---|
| Number | Compound | ¹HNMR | LCMS |
| I-129 | 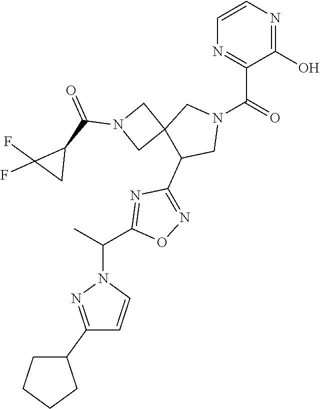 | ¹H NMR (400 MHz, Methanol-d₄) δ 7.80- 7.66 (m, 1H), 7.63-7.52 (m, 2H), 6.25-6.15 (m, 1H), 6.00-5.82 (m, 1H), 4.63-3.79 (m, 10H), 3.05 (d, J = 7.6 Hz, 1H), 2.61-2.43 (m, 1H), 2.06- 1.90 (m, 6H), 1.76 (d, J = 7.4 Hz, 3H), 1.67- 1.57 (m, 3H). | m/z = 596.2 [M + H]⁺ |

### Synthesis of (8-(5-(difluoro(4-(tetrahydro-2H-pyran-4-yl)phenyl)methyl)-1,3,4-oxadiazol-2-yl)-2-((R)-2,2-difluorocyclopropane-1-carbonyl)-2,6-diazaspiro[3.4]octan-6-yl)(5-hydroxypyrazin-2-yl)methanone (I-132)

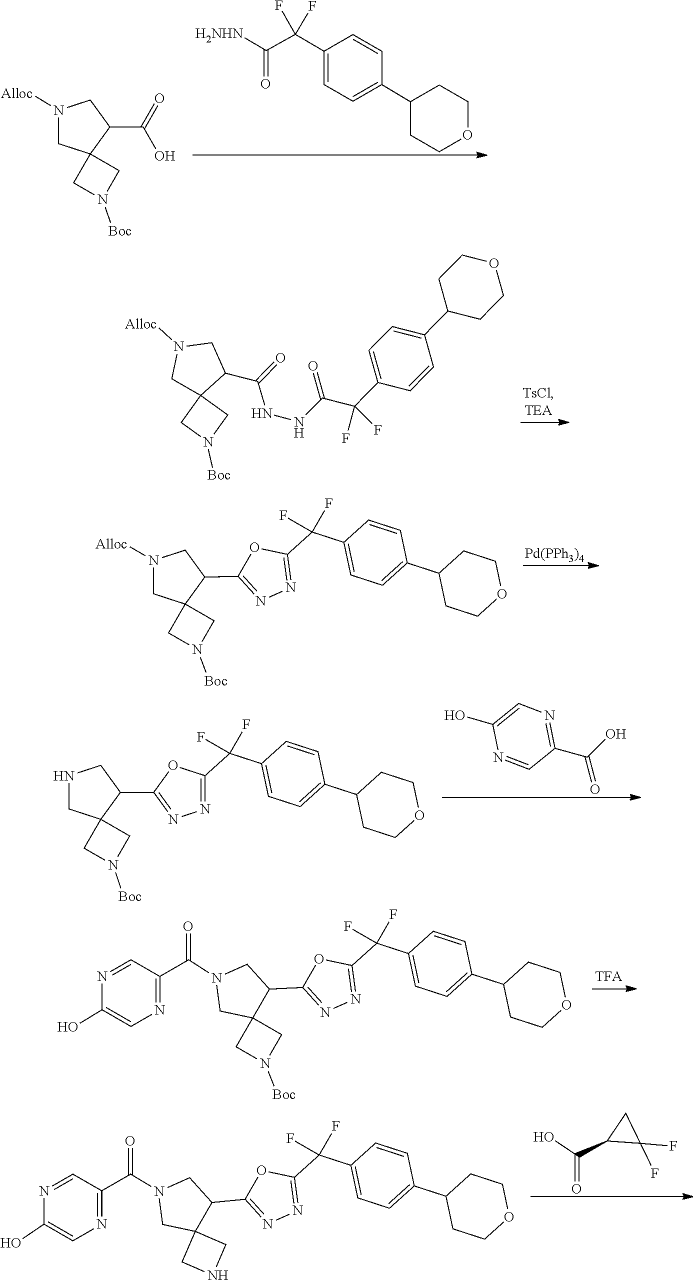

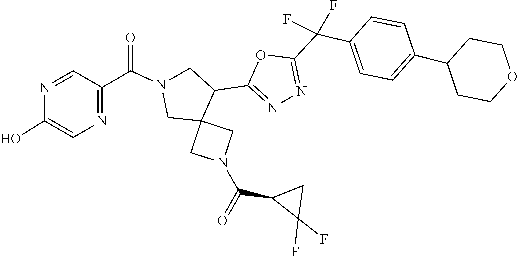 [SMILES: O=C(c1cnc(O)cn1)N1CC(c2nnc(C(F)(F)c3ccc(C4CCOCC4)cc3)o2)C2(C1)CN(C(=O)[C@H]1CC1(F)F)C2]

### Step 1: 6-allyl 2-(tert-butyl) 8-(2-(2,2-difluoro-2-(4-(tetrahydro-2H-pyran-4-yl)phenyl)acetyl)hydrazine-1-carbonyl)-2,6-diazaspiro[3.4]octane-2,6-dicarboxylate

To a solution of 6-((allyloxy)carbonyl)-2-(tert-butoxycarbonyl)-2,6-diazaspiro[3.4]octane-8-carboxylic acid (2.0 g, 5.88 mmol) in DCM (20 mL) was added HATU (3.35 g, 8.81 mmol) and DIPEA (3.1 mL, 17.36 mmol) and the mixture stirred at room temperature for 30 min. 2,2-difluoro-2-(4-(tetrahydro-2H-pyran-4-yl)phenyl)acetohydrazide (1.75 g, 6.46 mmol) was added and stirring continued another 2 h. The mixture was diluted with water (30 mL) and extracted with DCM (50 mL×2). The combined organic layers were washed with brine, dried over Na₂SO₄, filtered and concentrated. The residue obtained was purified by column chromatography on silica gel (eluent: DCM:MeOH=50:1 to DCM:MeOH=20:1) to afford 6-allyl 2-(tert-butyl) 8-(2-(2,2-difluoro-2-(4-(tetrahydro-2H-pyran-4-yl)phenyl)acetyl)hydrazine-1-carbonyl)-2,6-diazaspiro[3.4]octane-2,6-dicarboxylate (3.48 g, 75%) as a yellow solid. LCMS m/z=591.1 [M−H]⁻; ¹H NMR (400 MHz, CD₃OD) δ 7.70-7.53 (m, 2H), 7.43-7.31 (m, 2H), 6.05-5.85 (m, 1H), 5.39-5.16 (m, 2H), 4.63-4.51 (m, 2H), 3.88-3.50 (m, 9H), 3.03 (d, J=16.9 Hz, 2H), 2.95-2.82 (m, 3H), 1.87-1.72 (m, 4H), 1.45-1.40 (m, 9H).

### Step 2: 6-allyl 2-(tert-butyl) 8-(5-(difluoro(4-(tetrahydro-2H-pyran-4-yl)phenyl)methyl)-1,3,4-oxadiazol-2-yl)-2,6-diazaspiro[3.4]octane-2,6-dicarboxylate

To a solution of 6-allyl 2-(tert-butyl) 8-(2-(2,2-difluoro-2-(4-(tetrahydro-2H-pyran-4-yl)phenyl)acetyl)hydrazine-1-carbonyl)-2,6-diazaspiro[3.4]octane-2,6-dicarboxylate (2.6 g, 4.39 mmol) in DCM (30 mL) was added TEA (3.1 mL, 21.95 mmol) and TsCl (2.52 g, 13.17 mmol). The reaction mixture was stirred at room temperature overnight then was diluted with water (50 mL) and extracted with DCM (60 mL×3). The combined organic layers were washed with brine, dried over Na₂SO₄, filtered and concentrated. The residue obtained was purified by column chromatography on silica gel (eluent: DCM/MeOH=60/1) to afford 2-(tert-butyl) 8-(5-(difluoro(4-(tetrahydro-2H-pyran-4-yl)phenyl)methyl)-1,3,4-oxadiazol-2-yl)-2,6-diazaspiro[3.4]octane-2,6-dicarboxylate (1.3 g, 52%) as a brown solid. LCMS m/z=597.2 [M+Na]⁺; ¹H NMR 1H NMR (400 MHz, CD₃OD) δ 7.59 (d, J=8.1 Hz, 2H), 7.45 (d, J=8.1 Hz, 2H), 6.05-5.87 (m, 1H), 5.31 (d, J=17.3 Hz, 1H), 5.21 (d, J=10.6 Hz, 1H), 4.59 (d, J=5.4 Hz, 2H), 4.15-3.93 (m, 5H), 3.92-3.66 (m, 6H), 3.63-3.53 (m, 2H), 2.96-2.85 (m, 1H), 1.88-1.75 (m, 4H), 1.42 (s, 9H).

### Step 3: tert-butyl 8-(5-(difluoro(4-(tetrahydro-2H-pyran-4-yl)phenyl)methyl)-1,3,4-oxadiazol-2-yl)-2,6-diazaspiro[3.4]octane-2-carboxylate

To a solution of 2-(tert-butyl) 8-(5-(difluoro(4-(tetrahydro-2H-pyran-4-yl)phenyl)methyl)-1,3,4-oxadiazol-2-yl)-2,6-diazaspiro[3.4]octane-2,6-dicarboxylate (0.5 g, 0.87 mmol) in THF (10 mL) was added phenylsilane (0.47 g, 4.35 mmol) and Pd(PPh₃)₄ (2.52 g, 13.17 mmol). The reaction mixture was stirred at room temperature for 20 min then the solvent was removed and the residue obtained was purified by RP-column (eluent: CH₃CN:water=30:70) to afford tert-butyl 8-(5-(difluoro(4-(tetrahydro-2H-pyran-4-yl)phenyl)methyl)-1,3,4-oxadiazol-2-yl)-2,6-diazaspiro[3.4]octane-2-carboxylate (0.30 g, 70%) as a white solid. LCMS m/z=491.0 [M+H]⁺; ¹H NMR 1H NMR (400 MHz CD₃OD) δ 7.61 (d, J=8.0 Hz, 2H), 7.46 (d, J=8.0 Hz, 2H), 4.24-3.49 (m, 13H), 2.97-2.85 (m, 1H), 1.86-1.74 (m, 4H), 1.42 (d, J=1.6 Hz, 9H).

### Step 4: tert-butyl 8-(5-(difluoro(4-(tetrahydro-2H-pyran-4-yl)phenyl)methyl)-1,3,4-oxadiazol-2-yl)-6-(5-hydroxypyrazine-2-carbonyl)-2,6-diazaspiro[3.4]octane-2-carboxylate

To a solution of 5-hydroxypyrazine-2-carboxylic acid (17 mg, 0.12 mmol) in DCM (1.0 mL) was added HATU (46 mg, 0.12 mmol) and DIPEA (40 mg, 0.30 mmol) and the mixture stirred at room temperature for 30 min. Tert-butyl 8-(5-(difluoro(4-(tetrahydro-2H-pyran-4-yl)phenyl)methyl)-1,3,4-oxadiazol-2-yl)-2,6-diazaspiro[3.4]octane-2-carboxylate (50 mg, 0.10 mmol) was added and stirring continued for another 2 h. The solvent was removed under vacuum and the residue obtained was purified by RP-column (eluent: CH₃CN:water=60:40) to afford tert-butyl 8-(5-(difluoro(4-(tetrahydro-2H-pyran-4-yl)phenyl)methyl)-1,3,4-oxadiazol-2-yl)-6-(5-hydroxypyrazine-2-carbonyl)-2,6-diazaspiro[3.4]octane-2-carboxylate (42 mg, 69%) as a yellow solid. LCMS m/z=613.1 [M+H]⁺; ¹H NMR (400 MHz, CD₃OD) δ 8.09-8.00 (m, 1H), 7.64-7.54 (m, 2H), 7.50-7.39 (m, 2H), 4.45-4.29 (m, 2H), 4.18-3.77 (m, 9H), 3.65-3.52 (m, 2H), 2.90 (s, 1H), 1.87-1.76 (m, 4H), 1.50-1.39 (in, 9H).

### Step 5: (8-(5-(difluoro(4-(tetrahydro-2H-pyran-4-yl)phenyl)methyl)-1,3,4-oxadiazol-2-yl)-2,6-diazaspiro[3.4]octan-6-yl)(5-hydroxypyrazin-2-yl)methanone

To a solution of tert-butyl 8-(5-(difluoro(4-(tetrahydro-2H-pyran-4-yl)phenyl)methyl)-1,3,4-oxadiazol-2-yl)-6-(5-hydroxypyrazine-2-carbonyl)-2,6-diazaspiro[3.4]octane-2-carboxylate (130 mg, 0.21 mmol) in DCM (4.0 mL) was added TFA (0.5 mL) and the reaction stirred at room temperature for 2 h. The solvent was removed under vacuum to afford (8-(5-(difluoro(4-(tetrahydro-2H-pyran-4-yl)phenyl)methyl)-1,3,4-oxadiazol-2-yl)-2,6-diazaspiro[3.4]octan-6-yl)(5-hydroxypyrazin-2-yl)methanone (108 mg, quant) which was used directly in the next step. LCMS m/z=513.0 [M+H]⁺.

### Step 6: (8-(5-(difluoro(4-(tetrahydro-2H-pyran-4-yl)phenyl)methyl)-1,3,4-oxadiazol-2-yl)-2-((R)-2,2-difluorocyclopropane-1-carbonyl)-2,6-diazaspiro[3.4]octan-6-yl)(5-hydroxypyrazin-2-yl)methanone

To a solution of solution of (R)-2,2-difluorocyclopropane-1-carboxylic acid (28 mg, 0.23 mmol) in DCM (4.0 mL) was added HATU (87 mg, 0.23 mmol) and DTPEA (108 mg, 0.84 mmol) and the mixture stirred at room temperature for 30 min. (8-(5-(difluoro(4-(tetrahydro-2H-pyran-4-yl)phenyl)methyl)-1,3,4-oxadiazol-2-yl)-2,6-diazaspiro[3.4]octan-6-yl)(5-hydroxypyrazin-2-yl)methanone (108 mg, 0.21 mmol) was added and stirring continued for another 2 h. The solvent was removed under vacuum and the residue obtained was purified by prep-HPLC to afford 8-(5-(difluoro(4-(tetrahydro-2H-pyran-4-yl)phenyl)methyl)-1,3,4-oxadiazol-2-yl)-2-((R)-2,2-difluorocyclopropane-1-carbonyl)-2,6-diazaspiro[3.4]octan-6-yl)(5-hydroxypyrazin-2-yl)methanone (15 mg, 12%) as a white solid. LCMS m/z=617.1 [M+H]⁺; ¹H NMR (400 MHz, CD₃OD) δ 8.02 (d, J=10.0 Hz, 2H), 7.58 (t, J=7.9 Hz, 2H), 7.43 (d, J=7.8 Hz, 2H), 4.55-4.27 (m, 4H), 4.18-3.95 (m, 7H), 3.61-3.53 (m, 2H), 2.95-2.84 (m, 1H), 2.62-2.45 (m, 1H), 2.02-1.92 (m, 1H), 1.85-1.74 (m, 5H)

### Synthesis of 4-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)benzonitrile (I-133)

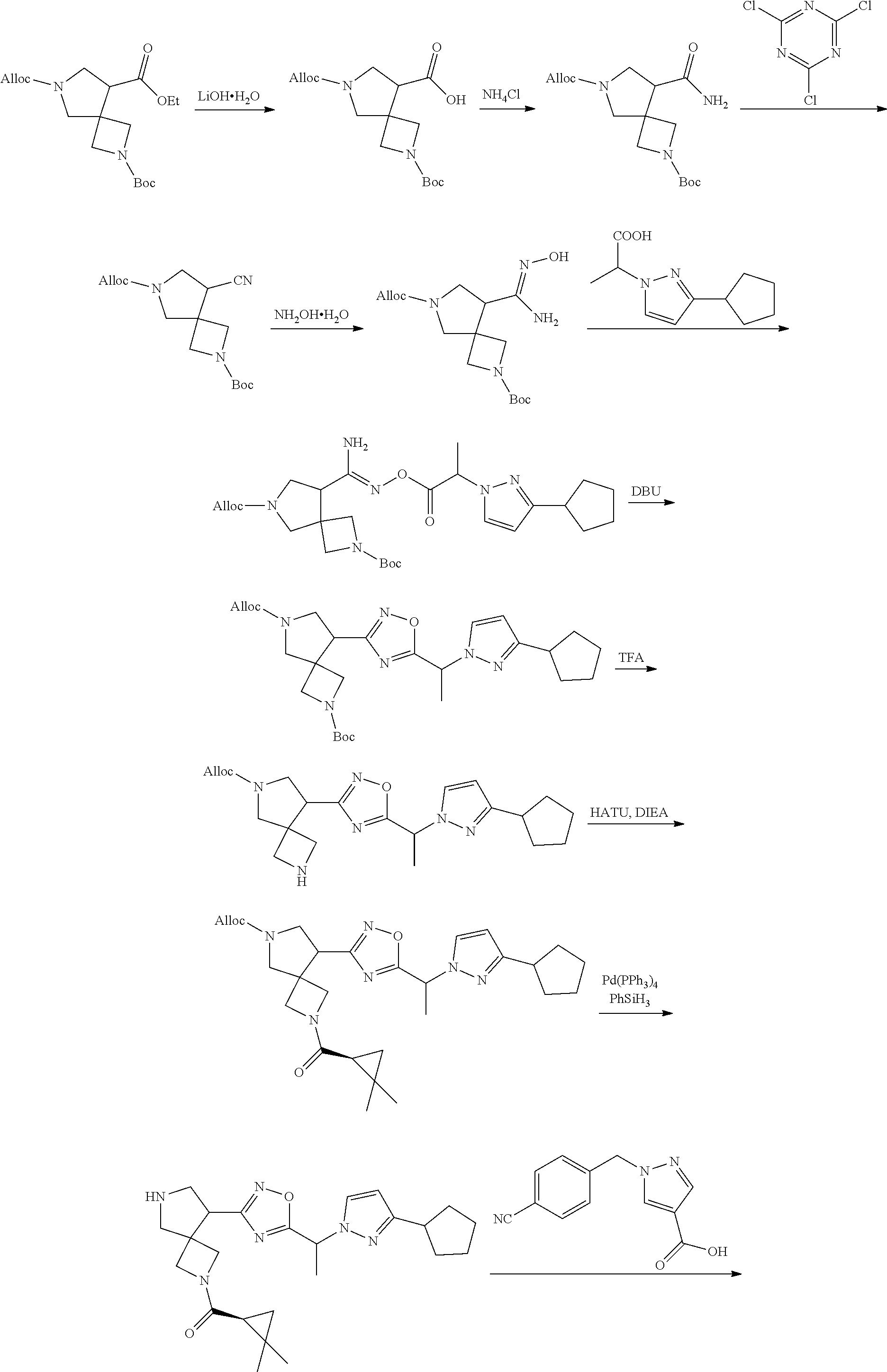

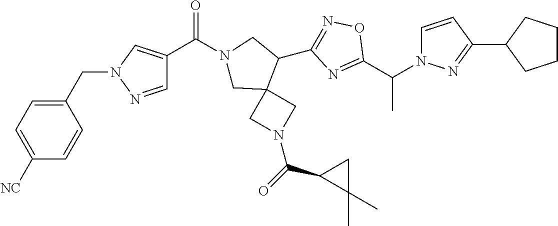 [SMILES: CC(c1nc(C2CN(C(=O)c3cnn(Cc4ccc(C#N)cc4)c3)CC23CN(C(=O)[C@H]2CC2(C)C)C3)no1)n1ccc(C2CCCC2)n1]

### Step 1: 6-((allyloxy)carbonyl)-2-(tert-butoxycarbonyl)-2,6-diazaspiro[3.4]octane-8-carboxylic acid

To a solution of 6-allyl 2-(tert-butyl) 8-ethyl 2,6-diazaspiro[3.4]octane-2,6,8-tricarboxylate (18.0 g, 48.86 mmol) in a mixture of THF and H₂O (160 mL/40 mL) was added LiOH (4.1 g, 97.71 mmol). The reaction was stirred at room temperature for 2 h then was diluted with water (100 mL) and extracted with ether (50 mL). The aqueous layer was collected, acidified to pH˜2 with 1M HCl and extracted with EtOAc (200 mL×3). The combined organic layers were washed with brine, dried over Na₂SO₄, filtered and concentrated to afford 6-((allyloxy)carbonyl)-2-(tert-butoxycarbonyl)-2,6-diazaspiro[3.4]octane-8-carboxylic acid (16.0 mg, 96%) as a white solid which was used directly in the next step. LCMS m/z=363.1 [M+Na]⁺; ¹H NMR (400 MHz, Chloroform-d) δ 9.34 (s, 1H), 6.02-5.82 (m, 1H), 5.38-5.15 (m, 2H), 4.59 (d, J=5.6 Hz, 2H), 4.08 (d, J=9.6 Hz, 1H), 4.02-3.91 (m, 1H), 3.90-3.58 (m, 6H), 3.11 (d, J=6.4 Hz, 1H), 1.43 (s, 9H).

### Step 2: 6-allyl 2-(tert-butyl) 8-carbamoyl-2,6-diazaspiro[3.4]octane-2,6-dicarboxylate

To a solution of 6-((allyloxy)carbonyl)-2-(tert-butoxycarbonyl)-2,6-diazaspiro[3.4]octane-8-carboxylic acid (16.0 g, 47.01 mmol) in DMF (200 mL) was added HATU (26.8 g, 70.5 mmol) and DIPEA (18.2 mg, 141.02 mmol) and the mixture stirred at room temperature for 30 min. NH₄Cl (7.5 g, 141.02 mmol) was added and the reaction stirred for another 3 h. The mixture was diluted with water (500 mL) and extracted with EtOAc (500 mL×4). The combined organic layers were washed with water and brine, dried over Na₂SO₄, filtered and concentrated to afford 6-allyl 2-(tert-butyl) 8-carbamoyl-2,6-diazaspiro[3.4]octane-2,6-dicarboxylate (20.0 g) as a brown solid. LCMS m/z=362.2 [M+Na]⁺; ¹H NMR (400 MHz, Chloroform-d) δ 6.24 (s, 1H), 6.04-5.89 (m, 1H), 5.45-5.20 (m, 2H), 4.61 (d, J=5.6 Hz, 2H), 4.11 (d, J=9.4 Hz, 1H), 3.94-3.57 (m, 6H), 3.02 (d, J=20.8 Hz, 1H), 2.91 (s, 1H), 2.83 (s, 1H), 1.46 (s, 9H).

### Step 3: 6-allyl 2-(tert-butyl) 8-cyano-2,6-diazaspiro[3.4]octane-2,6-dicarboxylate

To a solution of 6-allyl 2-(tert-butyl) 8-carbamoyl-2,6-diazaspiro[3.4]octane-2,6-dicarboxylate (15.9 g, 46.85 mmol) in DMF (200 mL) at 0° C. was added 2,4,6-trichloro-1,3,5-triazine (9.5 g, 51.53 mmol) and the reaction stirred for 2 h. The mixture was diluted with water (200 mL) and extracted with EtOAc (200 mL×3). The combined organic layers were washed with water and brine, dried over Na₂SO₄, filtered and concentrated. The mixture was purified by column chromatography on silica gel (eluent: Pet. Ether:EtOAc=1:1) to afford 6-allyl 2-(tert-butyl) 8-cyano-2,6-diazaspiro[3.4]octane-2,6-dicarboxylate (11.8 g, 79%) as a yellow oil. LCMS m/z=344.2 [M+Na]⁺; ¹H NMR (400 MHz, Chloroform-d) δ 5.99-5.84 (m, 1H), 5.37-5.17 (m, 2H), 4.62-4.57 (m, 2H), 4.21-4.06 (m, 1H), 4.02-3.80 (m, 3H), 3.80-3.63 (m, 4H), 3.21 (q, J=7.2 Hz, 1H), 1.44 (s, 9H).

### Step 4: 6-allyl 2-(tert-butyl) (Z)-8-(N′-hydroxycarbamimidoyl)-2,6-diazaspiro[3.4]octane-2,6-dicarboxylate

To a solution of 6-allyl 2-(tert-butyl) 8-cyano-2,6-diazaspiro[3, 4]octane-2, 6-dicarboxylate (11.8 g, 36.81 mmol) in EtOH (120 mL) was added NH₂OH H₂O (15.0 g, 147.24 mmol). The reaction was heated at 50° C. for 3 h then was diluted with water (100 mL) and extracted with EtOAc (150 mL×3). The combined organic layers were washed with brine, dried over Na₂SO₄, filtered and concentrated to afford 6-allyl 2-(tert-butyl) (Z)-8-(N′-hydroxycarbamimidoyl)-2,6-diazaspiro[3.4]octane-2,6-dicarboxylate (12.6 g, 97%) as a white solid. LCMS m/z=355.2 [M+H]⁺; ¹H NMR (400 MHz, Chloroform-d) δ 6.03-5.81 (m, 1H), 5.36-5.16 (m, 2H), 4.73-4.52 (m, 4H), 4.12 (d, J=9.0 Hz, 1H), 3.92-3.64 (m, 6H), 3.56 (dd, J=12.0, 7.6 Hz, 1H), 2.87 (s, 1H), 1.43 (s, 9H).

### Step 5: 6-allyl 2-(tert-butyl) (Z)-8-(N′-((2-(3-cyclopentyl-1H-pyrazol-1-yl)propanoyl) oxy)carbamimidoyl)-2,6-diazaspiro[3.4]octane-2,6-dicarboxylate

To a solution of 2-(3-cyclopentyl-1H-pyrazol-1-yl)propanoic acid (4.58 g, 22 mmol) in DMF (100 mL) was added HATU (8.37 g, 22 mmol) and DIPEA (6.56 g, 51 mmol) and the mixture stirred at room temperature for 30 min. 6-allyl 2-(tert-butyl) (Z)-8-(N′-hydroxycarbamimidoyl)-2,6-diazaspiro[3.4]octane-2,6-dicarboxylate (6.0 g, 17 mmol) was added and stirring continued for another 2 h. The mixture was diluted with water (250 mL) and extracted with EtOAc (300 mL×3). The combined organic layers were washed with brine, dried over Na₂SO₄, filtered and concentrated to afford crude 6-allyl 2-(tert-butyl) (Z)-8-(N′-((2-(3-cyclopentyl-1H-pyrazol-1-yl)propanoyl)oxy)carbamimidoyl)-2,6-diazaspiro[3.4]octane-2,6-dicarboxylate (9.4 g, quant.) which was used directly in the next step. LCMS m/z=545.2 [M+H]⁺.

### Step 6: 6-allyl 2-(tert-butyl) 8-(5-(1-(3-cyclopentyl-1H-pyrazol-1-yl)ethyl)-1,2,4-oxadiazol-3-yl)-2,6-diazaspiro[3.4]octane-2,6-dicarboxylate

To a solution of 6-allyl 2-(tert-butyl) 8-(N′-((2-(3-cyclopentyl-1H-pyrazol-1-yl)propanoyl)oxy)carbamimidoyl)-2,6-diazaspiro[3.4]octane-2,6-dicarboxylate (9.4 g, 17.3 mmol) in DMF (100 mL) was added DBU (3.15 g, 3.09 mmol). The reaction was heated at 90° C. for 2 h then was diluted with water (200 mL) and extracted with EtOAc (300 mL×3). The combined organic layers were washed with brine, dried over Na₂SO₄, filtered and concentrated. The residue was purified by column chromatography on silica gel (eluent: Pet. Ether:EtOAc=1:1, v/v) to afford 6-allyl 2-(tert-butyl) 8-(5-(1-(3-cyclopentyl-1H-pyrazol-1-yl)ethyl)-1,2,4-oxadiazol-3-yl)-2,6-diazaspiro[3.4]octane-2,6-dicarboxylate (7.2 g, 79%) as a colorless oil. LCMS n z=527.2 [M+H]⁺; ¹H NMR (400 MHz, DMSO-d₆) δ 7.78 (t, J=2.6 Hz, 1H), 6.13 (d, J=2.2 Hz, 1H), 6.06-5.85 (m, 2H), 5.38-5.15 (m, 2H), 4.52 (d, J=5.2 Hz, 2H), 4.04-3.92 (m, 1H), 3.89-3.77 (m, 2H), 3.77-3.51 (m, 6H), 3.00-2.86 (m, 1H), 2.01-1.77 (m, 5H), 1.72-1.47 (m, 6H), 1.35 (s, 9H).

### Step 7: allyl 8-(5-(1-(3-cyclopentyl-1H-pyrazol-1-yl)ethyl)-1,2,4-oxadiazol-3-yl)-2,6-diazaspiro [3.4]octane-6-carboxylate

To a solution of 6-allyl 2-(tert-butyl) 8-(5-(1-(3-cyclopentyl-1H-pyrazol-1-yl)ethyl)-1,2,4-oxadiazol-3-yl)-2,6-diazaspiro[3.4]octane-2,6-dicarboxylate (6.2 g, 11.8 mmol) in DCM (70 mL) was added TFA (35 mL) and the reaction stirred for 2 h. The solvent was removed under vacuum to afford allyl 8-(5-(1-(3-cyclopentyl-1H-pyrazol-1-yl)ethyl)-1,2,4-oxadiazol-3-yl)-2,6-diazaspiro[3.4]octane-6-carboxylate (10 g, quant.) as a yellow oil. LCMS m/z=427.2 [M+H]⁺.

### Step 8: allyl 8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carboxylate

To a solution of (S)-2,2-dimethylcyclopropane-1-carboxylic acid (1.67 g, 13.7 mmol) in DMF (60 mL) was added HATU (5.21 g, 13.7 mmol) and DIPEA (7.38 g, 57 mmol) and the mixture stirred at room temperature for 30 min. Allyl 8-(5-(1-(3-cyclopentyl-1H-pyrazol-1-yl)ethyl)-1,2,4-oxadiazol-3-yl)-2,6-diazaspiro[3.4]octane-6-carboxylate (4.87 g, 11.4 mmol) was added and stirring continued for another 2 h. The mixture was diluted with water (200 mL) and extracted with EtOAc (300 mL×3). The combined organic layers were washed with brine, dried over Na₂SO₄, filtered and concentrated. The residue was purified by column chromatography on silica gel (eluent: EtOAc) to afford allyl 8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carboxylate (4.8 g, 65%) as yellow semi-solid. LCMS m/z=523.2 [M+H]⁺; ¹H NMR (400 MHz, DMSO-d₆) δ 7.79 (d, J=7.2 Hz, 1H), 6.14 (d, J=2.2 Hz, 1H), 6.05-5.84 (m, 2H), 5.36-5.24 (m, 1H), 5.21-5.13 (m, 1H), 4.53 (d, J=5.2 Hz, 2H), 4.30-3.54 (m, 10H), 3.00-2.88 (m, 1H), 2.06-1.79 (m, 6H), 1.73-1.54 (m, 4H), 1.14-0.78 (m, 8H), 0.72-0.60 (m, 1H).

### Step 9: (8-(5-(1-(3-cyclopentyl-1H-pyrazol-1-yl)ethyl)-1,2,4-oxadiazol-3-yl)-2,6-diazaspiro[3.4]octan-2-yl)((S)-2,2-dimethylcyclopropyl)methanone

To a solution of allyl 8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carboxylate (1.0 g, 1.91 mmol) in THE (20 mL) was added Pd(PPh₃)₄ (0.221 g, 0.19 mmol) and phenylsilane (1.03 g, 9.5 mmol). The reaction was stirred at room temperature for 1 h then was diluted with water (20 mL) and extracted with EtOAc (80 mL×2). The combined organic layers were washed with brine, dried over Na₂SO₄, filtered and concentrated. The residue was purified by column chromatography on silica gel (eluent: DCM:MeOH=10:1) to afford (8-(5-(1-(3-cyclopentyl-1H-pyrazol-1-yl)ethyl)-1,2,4-oxadiazol-3-yl)-2,6-diazaspiro[3.4]octan-2-yl)((S)-2,2-dimethylcyclopropyl)methanone (520 mg, 56%) as a yellow solid. LCMS m/z=439.0 [M+H]⁺; ¹H NMR (400 MHz, Chloroform-d) δ 7.47 (dd, J=12.2, 2.2 Hz, 1H), 6.14 (d, J=2.6 Hz, 1H), 5.81-5.68 (m, 1H), 4.36-3.71 (m, 4H), 3.66-3.54 (m, 1H), 3.53-3.28 (m, 4H), 3.07 (q, J=8.0 Hz, 1H), 2.75-2.40 (m, 4H), 2.12-1.91 (m, 5H), 1.81-1.64 (m, 3H), 1.22-1.01 (m, 8H), 0.76-0.65 (m, 1H).

### Step 10: 4-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)benzonitrile

To a solution of 1-(4-cyanobenzyl)-1H-pyrazole-4-carboxylic acid (114 mg, 0.50 mmol) in DCM (2 mL) was added EDCI (121 mg, 0.63 mmol) and HOBt (85 mg, 0.63 mmol) and DIPEA (163 mg, 1.26 mmol). The mixture was stirred at room temperature for 30 min then (8-(5-(1-(3-cyclopentyl-1H-pyrazol-1-yl)ethyl)-1,2,4-oxadiazol-3-yl)-2,6-diazaspiro[3.4]octan-2-yl)((S)-2,2-dimethylcyclopropyl)methanone (185 mg, 0.42 mmol) was added. The reaction was stirred at room temperature overnight then was diluted with water (40 mL) and extracted with DCM (60 mL×2). The combined organic layers were washed with brine, dried over Na₂SO₄, filtered and concentrated. The residue obtained was purified by prep-HPLC to afford 4-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)benzonitrile (30 mg, 30%) as a white solid. LCMS m/z=648.3 [M+H]⁺; ¹H NMR (400 MHz, CD₃OD) δ 8.30 (d, J=11.2 Hz, 1H), 7.95 (d, J=10.8 Hz, 1H), 7.74-7.65 (m, 3H), 7.41 (t, J=7.4 Hz, 2H), 6.16 (s, 1H), 5.92-5.81 (m, 1H), 5.48 (d, J=2.6 Hz, 2H), 4.57-3.81 (m, 9H), 3.09-2.96 (m, 1H), 2.06-1.88 (m, 5H), 1.80-1.52 (m, 6H), 1.46-1.26 (m, 1H), 1.21-0.99 (m, 7H), 0.80-0.70 (m, 1H).

Table 30: The compounds listed in Table 30 were synthesized from (8-(5-(1-(3-cyclopentyl-1H-pyrazol-1-yl)ethyl)-1,2,4-oxadiazol-3-yl)-2,6-diazaspiro[3.4]octan-2-yl)((S)-2,2-dimethylcyclopropyl)methanone according to the procedures outlined for I-133 using the appropriate commercially available reagents and/or intermediates described elsewhere.

TABLE 30

| Example | | | |
|---|---|---|---|
| Number | Compound | ¹HNMR | LCMS |
| I-118 | 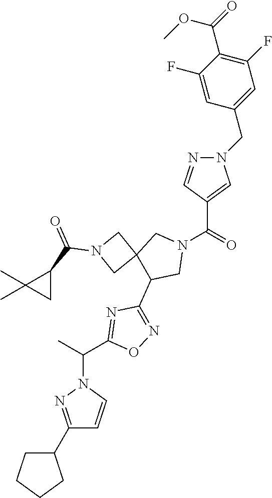 | ¹H NMR (400 MHz, Methanol-d₄) δ 8.31 (d, J = 9.4 Hz, 1H), 7.98 (d, J = 9.6 Hz, 1H), 7.77 − 7.67 (m, 1H), 6.96 (t, J = 8.2 Hz, 2H), 6.20 − 6.11 (m, 1H), 5.91 − 5.81 (m, 1H), 5.53 − 5.37 (m, 2H), 4.59 − 3.80 (m, 12H), 3.08 − 2.95 (m, 1H), 2.06 − 1.90 (m, 5H), 1.83 − 1.53 (m, 6H), 1.45 − 1.28 (m, 1H), 1.21 − 0.98 (m, 7H), 0.82 − 0.71 (m, 1H). | m/z = 703.1 [M + H]⁺ |
| I-117 | 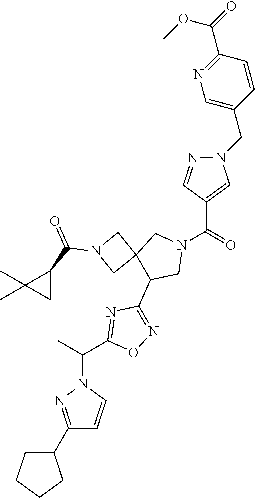 [SMILES: COC(=O)c1ccc(Cn2cc(C(=O)N3CC(c4noc(C(C)n5ccc(C6CCCC6)n5)n4)C4(C3)CN(C(=O)[C@H]3CC3(C)C)C4)cn2)cn1] | ¹H NMR (400 MHz, Chloroform-d) δ 8.68 (d, J = 4.0 Hz, 1H), 8.12 (d, J = 8.5 Hz, 1H), 7.93 (s, 1H), 7.85 (s, 1H), 7.69 (t, J = 6.3 Hz, 1H), 7.45 (s, 1H), 6.13 (s, 1H), 5.79 − 5.68 (m, 1H), 4.41 − 3.63 (m, 12H), 3.11 − 2.99 (m, 1H), 2.07 − 1.92 (m, 5H), 1.76 − 1.54 (m, 9H), 1.19 − 1.05 (m, 7H), 0.78 − 0.69 (m, 1H). | m/z = 682.0 [M + H]⁺ |
| I-113 | 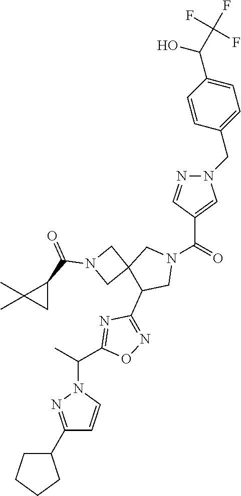 [SMILES: CC(c1nc(C2CN(C(=O)c3cnn(Cc4ccc(C(O)C(F)(F)F)cc4)c3)CC23CN(C(=O)[C@H]2CC2(C)C)C3)no1)n1ccc(C2CCCC2)n1] | ¹H NMR (400 MHz, DMSO-d₆) δ8.39 (d, J = 13.6 Hz, 1H), 7.88 − 7.74 (m, 2H), 7.46 (d, J = 7.8 Hz, 2H), 7.29 (d, J = 6.9 Hz, 2H), 6.81 (d, J = 5.4 Hz, 1H), 6.12 (d, J = 2.4 Hz, 1H), 5.99 (s, 1H), 5.38 (s, 2H), 5.14 (m, 1H), 4.41 − 4.05 (m, 3H), 3.99 (m, 2H), 3.80 (m, 4H), 2.95 (m, 1H), 1.93 − 1.81 (m, 5H), 1.60 (d, J = 41.2 Hz, 6H), 1.29 (m, 1H), 1.13 − 0.92 (m, 6H), 0.84 (m, 1H), 0.65 (m, 1H). | m/z = 721.3 [M + H]⁺ |

### Synthesis of 4-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)-2,6-difluorobenzoic acid (I-130)

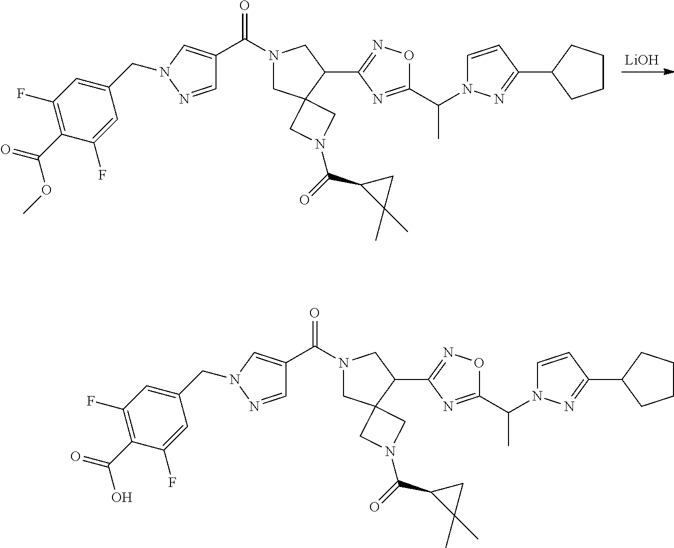

### Step 1: 4-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)-2,6-difluorobenzoic acid

To a solution of methyl 4-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)-2,6-difluorobenzoate (90 mg, 0.125 mmol) in a mixture of THE and H₂O (5 mL/1 mL) was added lithium hydroxide monohydrate (10.54 mg, 0.251 mmol). The reaction mixture was stirred at room temperature for 2 h, then was diluted with water (40 mL) and extracted with ether (50 mL). The aqueous layer was collected, acidified to pH˜2 with 1M HCl and extracted with EtOAc (50 mL×2). The combined organic layers were washed with brine, dried over Na₂SO₄, filtered and concentrated. The residue was purified by prep-HPLC to afford 4-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)-2,6-difluorobenzoic acid (65 mg, 74%) as a white solid. LCMS m/z=703.1 [M+H]⁺; ¹H NMR (400 MHz, DMSO-d₆) δ 8.55-8.38 (m, 1H), 7.96-7.83 (m, 1H), 7.82-7.72 (m, 1H), 7.05 (dd, J=9.2, 3.2 Hz, 2H), 6.17-6.09 (m, 1H), 6.05-5.93 (m, 1H), 5.52-5.34 (m, 2H), 4.40-3.93 (m, 5H), 3.91-3.69 (m, 4H), 3.02-2.87 (m, 1H), 1.95-1.79 (m, 5H), 1.71-1.47 (m, 6H), 1.39-1.21 (m, 1H), 1.13-0.91 (m, 6H), 0.87-0.80 (m, 1H), 0.72-0.61 (m, 1H).

Table 31: The compounds listed in Table 31 were synthesized from the corresponding esters in Table 30 according to the procedures outlined for I-130 using the appropriate commercially available reagents and/or intermediates described elsewhere.

TABLE 31

| Example | | | |
|---|---|---|---|
| Number | Compound | ¹HNMR | LCMS |
| I-126 | 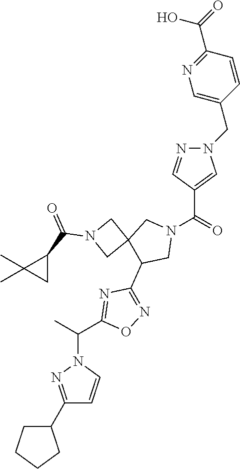 [SMILES: CC(c1nc(C2CN(C(=O)c3cnn(Cc4ccc(C(=O)O)nc4)c3)CC23CN(C(=O)[C@H]2CC2(C)C)C3)no1)n1ccc(C2CCCC2)n1] | ¹H NMR (400 MHz, Chloroform-d) δ 8.67 (s, 1H), 8.11 (s, 1H), 7.93 (s, 1H), 7.85 (s, 1H), 7.67 (s, 1H), 7.45 (s, 1H), 6.13 (s, 1H), 5.73 (s, 1H), 5.42 (s, 2H), 4.01 (s, 8H), 3.06 (s, 1H), 2.05-1.89 (m, 5H), 1.60 (s, 8H), 1.12 (s, 7H), 0.70 (s, 1H). | m/z = 668.2 [M + H]⁺ |
| I-127 | 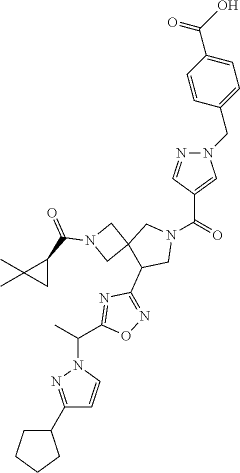 [SMILES: CC(c1nc(C2CN(C(=O)c3cnn(Cc4ccc(C(=O)O)cc4)c3)CC23CN(C(=O)[C@H]2CC2(C)C)C3)no1)n1ccc(C2CCCC2)n1] | ¹H NMR (400 MHz, DMSO-d₆) δ 12.96 (s, 1H), 8.42 (d, J = 15.0 Hz, 1H), 7.94 − 7.83 (m, 3H), 7.78 (s, 1H), 7.37 − 7.28 (m, 2H), 6.12 (d, J = 2.2 Hz, 1H), 5.99 (m, 1H), 5.45 (s, 2H), 4.35 − 3.73 (m, 9H), 2.96 (m, 1H), 1.92 − 1.81 (m, 5H), 1.60 (m, 6H), 1.39 − 1.24 (m, 1H), 1.12 − 0.93 (m, 6H), 0.84 (s, 1H), 0.66 (m, 1H). | m/z = 667.3 [M + H]⁺ |
| I-131 | 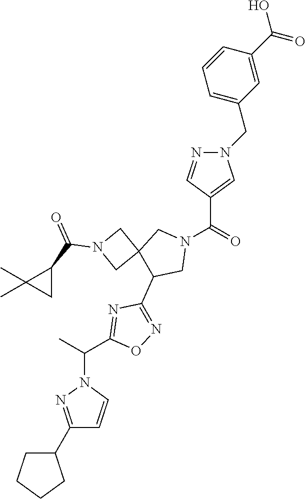 [SMILES: CC(c1nc(C2CN(C(=O)c3cnn(Cc4cccc(C(=O)O)c4)c3)CC23CN(C(=O)[C@H]2CC2(C)C)C3)no1)n1ccc(C2CCCC2)n1] | ¹H NMR (400 MHz, DMSO-d₆) δ 8.43 (d, J = 14.8 Hz, 1H), 7.91 − 7.82 (m, 3H), 7.78 (d, J = 6.8 Hz, 1H), 7.49 (d, J = 9.6 Hz, 2H), 6.12 (d, J = 2.4 Hz, 1H), 5.98 (s, 1H), 5.44 (s, 2H), 4.42 − 4.04 (m, 3H), 4.01 (d, J = 14.8 Hz, 2H), 3.83 (dd, J = 19.8, 13.3 Hz, 3H), 3.72 (s, 1H), 2.95 (s, 1H), 1.94 − 1.80 (m, 5H), 1.70 − 1.46 (m, 6H), 1.30 − 1.25 (m, 1H), 1.13 − 0.92 (m, 6H), 0.84 (s, 1H), 0.66 (d, J = 12.2 Hz, 1H). | m/z = 667.2 [M + H]⁺ |
| I-124 | 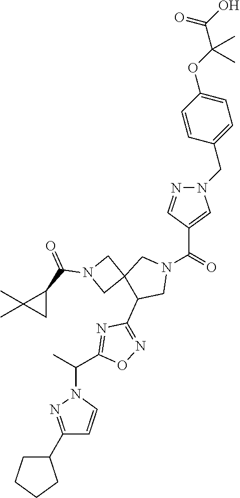 [SMILES: CC(c1nc(C2CN(C(=O)c3cnn(Cc4ccc(OC(C)(C)C(=O)O)cc4)c3)CC23CN(C(=O)[C@H]2CC2(C)C)C3)no1)n1ccc(C2CCCC2)n1] | ¹H NMR (400 MHz, DMSO-d6) δ 8.34 (d, J = 15.6 Hz, 1H), 7.86 − 7.74 (m, 2H), 7.23 − 7.15 (m, 2H), 6.78 (d, J = 8.2 Hz, 2H), 6.12 (s, 1H), 6.05 − 5.93 (m, 1H), 5.26 (s, 2H), 4.41 − 4.21 (m, 1H), 4.15 − 3.93 (m, 4H), 3.91 − 3.67 (m, 4H), 3.01 − 2.89 (m, 1H), 1.95 − 1.77 (m, 5H), 1.70 − 1.51 (m, 6H), 1.49 (s, 6H), 1.39 − 1.21 (m, 1H), 1.13 − 0.92 (m, 6H), 0.88 − 0.80 (m, 1H), 0.71 − 0.60 (m, 1H). | m/z = 725.3 [M + H]⁺ |
| I-123 | 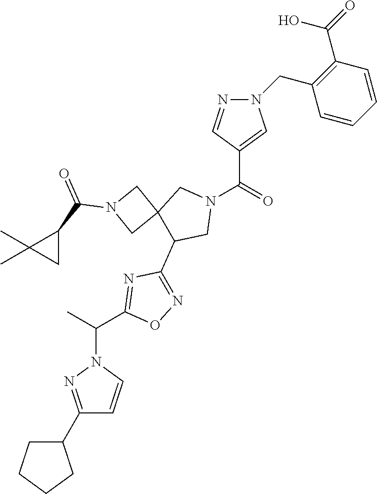 [SMILES: CC(c1nc(C2CN(C(=O)c3cnn(Cc4ccccc4C(=O)O)c3)CC23CN(C(=O)[C@H]2CC2(C)C)C3)no1)n1ccc(C2CCCC2)n1] | ¹HNMR (400 MHz, DMSO-d₆) δ 8.34 (d, J = 18.2 Hz, 1H), 7.98 − 7.83 (m, 2H), 7.77 (s, 1H), 7.51 (s, 1H), 7.43 (d, J = 7.6 Hz, 1H), 6.78 (t, J = 5.8 Hz, 1H), 6.13 (s, 1H), 6.00 (d, J = 6.4 Hz, 1H), 5.76 (s, 2H), 4.41 − 4.20 (m, 1H), 4.20 − 3.95 (m, 4H), 3.91 − 3.68 (m, 4H), 3.03 − 2.88 (m, 1H), 1.96 − 1.78 (m, 5H), 1.70 − 1.47 (m, 6H), 1.32 (d, J = 18.6 Hz, 1H), 1.14 − 0.91 (m, 6H), 0.84 (s, 1H), 0.66 (d, J = 10.4 Hz, 1H). | m/z = 667.2 [M + H]⁺ |

### Synthesis of 4-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)-2-hydroxybenzoic acid (I-122)

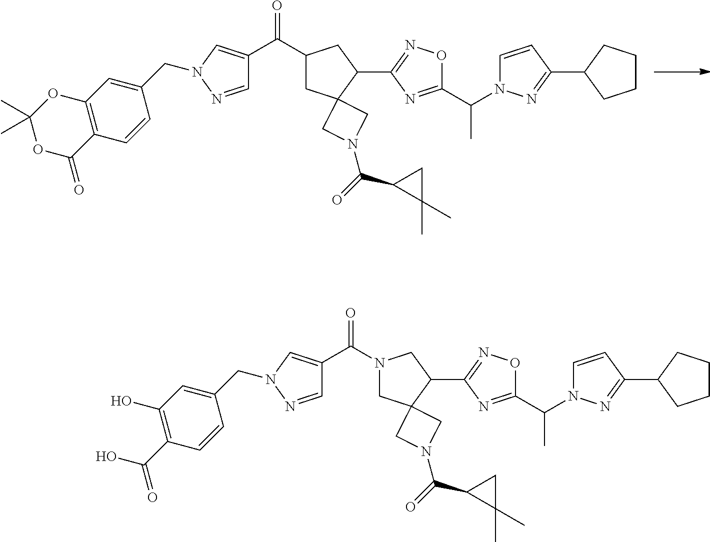 [SMILES: CC(c1nc(C2CC(C(=O)c3cnn(Cc4ccc5c(c4)OC(C)(C)OC5=O)c3)CC23CN(C(=O)[C@H]2CC2(C)C)C3)no1)n1ccc(C2CCCC2)n1 ; CC(c1nc(C2CN(C(=O)c3cnn(Cc4ccc(C(=O)O)c(O)c4)c3)CC23CN(C(=O)[C@H]2CC2(C)C)C3)no1)n1ccc(C2CCCC2)n1]

### 7-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)-2,2-dimethyl-4H-benzo[d][1,3]dioxin-4-one

7-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)-2,2-dimethyl-4H-benzo[d][1,3]dioxin-4-one was synthesized from (8-(5-(1-(3-cyclopentyl-1H-pyrazol-1-yl)ethyl)-1,2,4-oxadiazol-3-yl)-2,6-diazaspiro[3.4]octan-2-yl)((S)-2,2-dimethylcyclopropyl)methanone according to the procedures outlined for I-133 using the appropriate commercially available reagents and/or intermediates described elsewhere. ¹H NMR (400 MHz, Chloroform-d) δ 8.06-7.82 (m, 3H), 7.47 (d, J=6.4 Hz, 1H), 7.04-6.88 (m, 1H), 6.75 (d, J=10.4 Hz, 1H), 6.13 (s, 1H), 5.83-5.64 (m, 1H), 5.35 (d, J=7.1 Hz, 2H), 5.30-5.26 (m, 1H), 4.30-3.63 (m, 8H), 3.27-2.91 (m, 2H), 2.06-1.89 (m, 3H), 1.61 (s, 3H), 1.46-1.36 (m, 14H), 1.18-0.99 (m, 7H), 0.73 (s, 1H).

### Step 1: 4-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)-2-hydroxybenzoic acid

To a solution of 7-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)-2,2-dimethyl-4H-benzo[d][1,3]dioxin-4-one (70 mg, 0.1 mmol) in a mixture of THF and water (3 mL) was added NaOH (20 mg, 0.5 mmol). The reaction was heated at 60° C. for 4 h then the solvent was removed and the residue obtained purified by RP-column to afford 4-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)-2-hydroxybenzoic acid (36 mg, 55%) as white solid. LCMS: m/z=683.3 [M+H]⁺; ¹H NMR (400 MHz, CD₃OD) δ 8.26 (d, J=12.2 Hz, 1H), 7.96 (d, J=10.9 Hz, 1H), 7.82 (d, J=10.9 Hz, 1H), 7.68 (s, 1H), 6.82-6.71 (m, 2H), 6.16 (s, 1H), 5.92-5.80 (m, 1H), 5.38 (s, 2H), 4.39-3.80 (m, 9H), 3.04 (d, J=8.0 Hz, 1H), 2.07-1.86 (m, 5H), 1.82-1.52 (m, 6H), 1.44-1.27 (m, 1H), 1.19-0.99 (m, 7H), 0.74 (s, 1H).

### Synthesis of (1-(4-(1H-tetrazol-5-yl)benzyl)-1H-pyrazol-4-yl)(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)methanone (I-121)

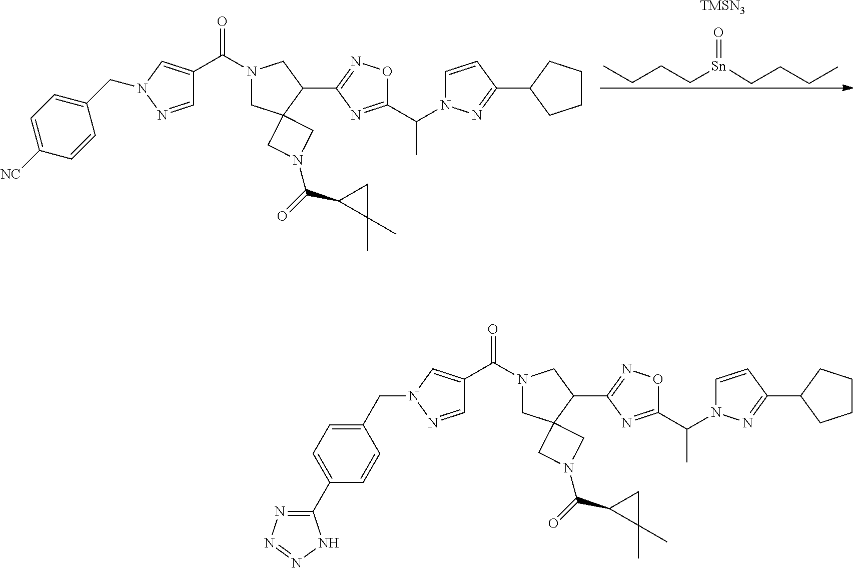

### Step 1: (1-(4-(1H-tetrazol-5-yl)benzyl)-1H-pyrazol-4-yl)(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)methanone

A mixture of 4-((4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)benzonitrile (100 mg, 0.20 mmol), dibutylstannanone (8 mg, 0.03 mmol) and TMSN₃ (35 mg, 0.30 mmol) in toluene (1 mL) was heated at 100° C. for 12 hours. The mixture was concentrated directly and purified by prep-HPLC to afford (1-(4-(1H-tetrazol-5-yl)benzyl)-1H-pyrazol-4-yl)(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)methanone (40 mg, 39%) as a white solid. LCMS m/z=691.2 [M+H]⁺; ¹H NMR (400 MHz, CD₃OD) δ 8.31 (d, J=13.2 Hz, 1H), 8.03-7.91 (m, 3H), 7.71-7.64 (m, 1H), 7.48-7.42 (m, 2H), 6.15 (s, 1H), 5.91-5.80 (m, 1H), 5.47 (s, 2H), 4.56-3.81 (m, 9H), 3.07-2.91 (m, 1H), 2.03-1.84 (m, 5H), 1.81-1.51 (m, 6H), 1.45-0.96 (m, 9H), 0.82-0.67 (m, 1H).

### Synthesis of (1-((1H-tetrazol-5-yl)methyl)-1H-pyrazol-4-yl)(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)methanone (I-114)

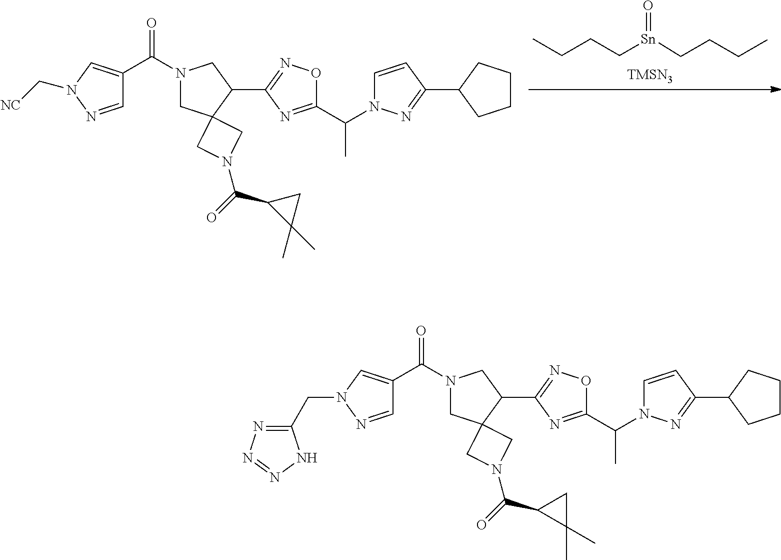

(1-((1H-tetrazol-5-yl)methyl)-1H-pyrazol-4-yl)(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)methanone was synthesized from 2-(4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)acetonitrile (110 mg, 0.19 mmol), dibutylstannanone according to the procedures outlined for I-121 using the appropriate commercially available reagents and/or intermediates described elsewhere. LCMS m/z=615.3 [M+H]⁺; ¹H NMR (400 MHz, CD₃OD) δ 8.34 (d, J=11.2 Hz, 1H), 7.95 (d, J=8.2 Hz, 1H), 7.72-7.65 (m, 1H), 6.17 (s, 1H), 5.92-5.82 (m, 1H), 5.78 (s, 2H), 4.58-3.80 (m, 9H), 3.11-2.96 (m, 1H), 2.07-1.90 (m, 5H), 1.81-1.54 (m, 6H), 1.47-1.27 (m, 2H), 1.21-0.99 (m, 7H), 0.81-0.71 (m, 1H).

### Synthesis of 2-(4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)acetonitrile (I-119)

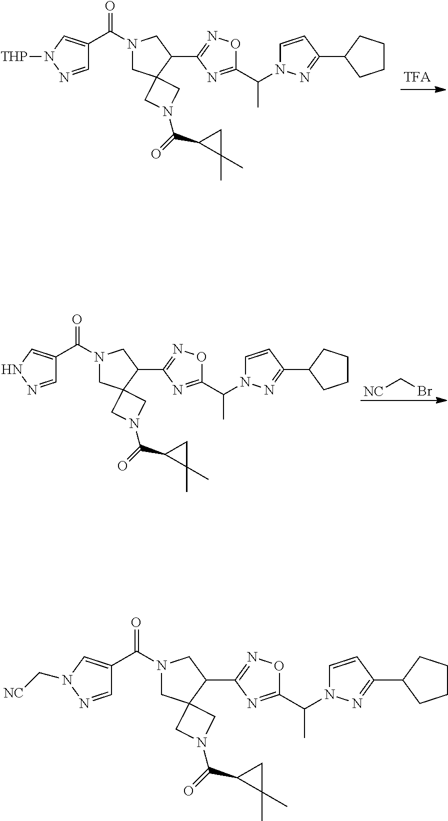

### (8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(1-(tetrahydro-2H-pyran-2-yl)-1H-pyrazol-4-yl)methanone

(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(1-(tetrahydro-2H-pyran-2-yl)-1H-pyrazol-4-yl)methanone was synthesized from (8-(5-(1-(3-cyclopentyl-1H-pyrazol-1-yl)ethyl)-1,2,4-oxadiazol-3-yl)-2,6-diazaspiro[3.4]octan-2-yl)((S)-2,2-dimethylcyclopropyl)methanone according to the procedures outlined for I-133 using the appropriate commercially available reagents and/or intermediates described elsewhere. LCMS m/z=617.2 [M+H]⁺; ¹H NMR (400 MHz, DMSO-d₆) δ 8.31 (d, J=12.7 Hz, 1H), 7.90-7.71 (m, 2H), 6.13 (s, 1H), 5.98 (s, 1H), 5.44 (d, J=9.8 Hz, 1H), 4.31-3.59 (m, 12H), 2.16-1.77 (m, 9H), 1.72-1.47 (m, 9H), 1.41-0.81 (m, 12H), 0.73-0.60 (m, 1H).

### Step 1: (8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(1H-pyrazol-4-yl)methanone

To a solution of (8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(1-(tetrahydro-2H-pyran-2-yl)-1H-pyrazol-4-yl)methanone (500 mg, 0.81 mmol) in DCM (5 mL) was added TFA (2.5 mL) and the reaction stirred at room temperature for 1 h. The reaction was concentrated under vacuum to afford (8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(1H-pyrazol-4-yl)methanone which was used directly in the next step. LCMS m/z=533.3 [M+H]⁺; ¹H NMR (400 MHz, DMSO-d₆) δ 8.02 (d, J=16.6 Hz, 2H), 7.77 (s, 1H), 6.13 (s, 1H), 5.99 (s, 1H), 5.74 (s, 2H), 1.94-1.43 (m, 15H), 1.37-1.17 (m, 7H), 1.14-0.80 (m, 9H), 0.72-0.59 (m, 1H).

### Step 2: 2-(4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)acetonitrile

To a solution of (8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(1H-pyrazol-4-yl)methanone (0.8 g, 0.65 mmol) and 2-bromoacetonitrile (117 mg, 0.97 mmol) in DMF (5 mL) was added K₂CO₃ (270 mg, 1.95 mmol). The reaction was stirred at room temperature under a N₂ atmosphere overnight then was concentrated. The residue obtained was purified by prep-HPLC to afford 2-(4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)acetonitrile (38 mg, 10%) as a white solid. LCMS m/z=572.3 [M+H]⁺; ¹H NMR (400 MHz, CD₃OD) δ 8.27 (d, J=10.2 Hz, 1H), 7.99 (d, J=9.8 Hz, 1H), 7.75-7.63 (m, 1H), 6.17 (s, 1H), 5.88 (s, 1H), 5.38 (s, 2H), 4.58-3.80 (m, 9H), 3.11-2.96 (m, 1H), 2.10-1.90 (m, 5H), 1.83-1.52 (m, 6H), 1.47-1.27 (m, 1H), 1.20-0.99 (m, 7H), 0.82-0.71 (m, 1H).

### Synthesis of (2-((R)-2,2-difluorocyclopropane-1-carbonyl)-8-(5-(1-(3-(tetrahydro-2H-pyran-4-yl)-1H-pyrazol-1-yl)ethyl)-1,2,4-oxadiazol-3-yl)-2,6-diazaspiro[3.4]octan-6-yl)(5-hydroxypyrazin-2-yl)methanone) (I-116)

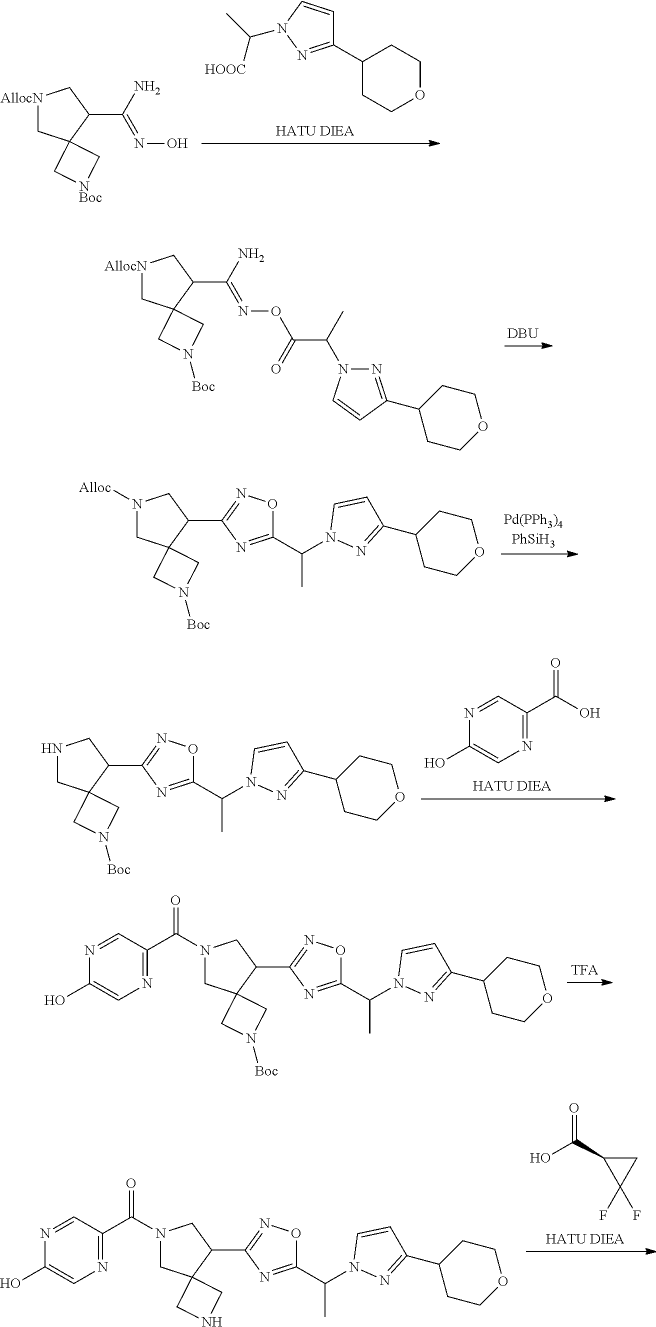

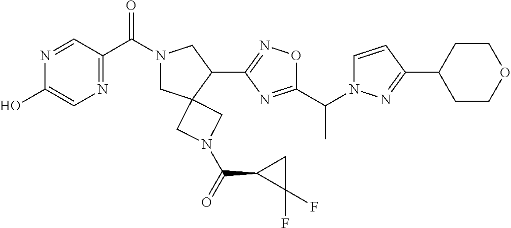 [SMILES: CC(c1nc(C2CN(C(=O)c3cnc(O)cn3)CC23CN(C(=O)[C@H]2CC2(F)F)C3)no1)n1ccc(C2CCOCC2)n1]

### Step 1: 6-allyl 2-(tert-butyl) (Z)-8-(N′-((2-(3-(tetrahydro-2H-pyran-4-yl)-1H-pyrazol-1-yl)propanoyl)oxy)carbamimidoyl)-2,6-diazaspiro[3.4]octane-2,6-dicarboxylate

To a solution of 2-(3-(tetrahydro-2H-pyran-4-yl)-1H-pyrazol-1-yl)propanoic acid (608 mg, 2.71 mmol) in DMF (10 mL) was added HATU (1.03 g, 2.71 mmol) and DIPEA (876 mg, 6.77 mmol) and the mixture stirred at room temperature for 30 min. 6-allyl 2-(tert-butyl) (Z)-8-(N′-hydroxycarbamimidoyl)-2,6-diazaspiro[3.4]octane-2,6-dicarboxylate (0.8 g, 2.26 mmol) was added and stirring continued for another 2 h. The mixture was diluted with water (30 mL) and extracted with EtOAc (50 mL×3). The combined organic layers were washed with brine, dried over Na₂SO₄, filtered and concentrated to afford crude 6-allyl 2-(tert-butyl) (Z)-8-(N′-((2-(3-(tetrahydro-2H-pyran-4-yl)-1H-pyrazol-1-yl)propanoyl)oxy)carbamimidoyl)-2,6-diazaspiro[3.4]octane-2,6-dicarboxylate (1.5 g, quant.) as a yellow oil which was used directly in the next step. LCMS m/z=561.3 [M+H]⁺.

### Step 2: 6-allyl 2-(tert-butyl) 8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2,6-dicarboxylate

To a solution of crude 6-allyl 2-(tert-butyl) (Z)-8-(N′-((2-(3-(tetrahydro-2H-pyran-4-yl)-1H-pyrazol-1-yl)propanoyl)oxy)carbamimidoyl)-2,6-diazaspiro[3.4]octane-2,6-dicarboxylate (1.27 g, 2.27 mmol) in DMF (10 mL) was added DBU (0.4 mL, 2.72 mmol). The reaction mixture was heated at 90° C. for 2 h then was diluted with water (30 mL) and extracted with EtOAc (50 mL×3). The combined organic layers was washed with brine, dried over Na₂SO₄, filtered and concentrated. The residue was purified by column chromatography on silica gel (eluent: Pet. Ether:EtOAc=1:2, v/v) to afford 6-allyl 2-(tert-butyl) 8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2,6-dicarboxylate (750 mg, 61%) as a white semi-solid. LCMS m/z=543.2[M+H]⁺; ¹H NMR (400 MHz, Chloroform-d) δ 7.55-7.49 (m, 1H), 6.19 (d, J=2.4 Hz, 1H), 6.01-5.89 (m, 1H), 5.77 (q, J=7.2 Hz, 1H), 5.33 (d, J=1.0 Hz, 1H), 5.25 (dd, J=10.6, 5.4 Hz, 1H), 4.63 (d, J=5.4 Hz, 2H), 4.14-3.99 (m, 3H), 3.98-3.89 (m, 2H), 3.89-3.71 (m, 5H), 3.66-3.49 (m, 3H), 3.01-2.88 (m, 1H), 2.01 (d, J=7.2 Hz, 3H), 1.95-1.71 (m, 4H), 1.45 (s, 9H).

### Step 3: tert-butyl 8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2-carboxylate

To a solution of 6-allyl 2-(tert-butyl) 8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2,6-dicarboxylate (700 mg, 1.29 mmol) in THE (10 mL) was added Pd(PPh₃)₄ (0.149 g, 0.129 mmol) and phenylsilane (0.7 g, 6.45 mmol). The reaction was stirred at room temperature for 1 h then was diluted with water (20 mL) and extracted with EtOAc (80 mL×2). The combined organic layers were washed with brine, dried over Na₂SO₄, filtered and concentrated. The residue was purified by column chromatography on silica gel (eluent: DCM:MeOH=10:1) to afford tert-butyl 8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2-carboxylate (350 mg, 59%) as a yellow solid. LCMS m/z=459.2 [M+H]⁺; ¹H NMR (400 MHz, Chloroform-d) δ 7.54-7.45 (m, 1H), 6.16 (d, J=2.4 Hz, 1H), 5.73 (q, J=7.2 Hz, 1H), 4.06-3.93 (m, 3H), 3.91-3.82 (m, 1H), 3.69 (q, J=9.2 Hz, 2H), 3.55-3.44 (m, 3H), 3.37-3.20 (m, 4H), 2.96-2.83 (m, 1H), 1.97 (d, J=7.2 Hz, 3H), 1.89-1.71 (m, 5H), 1.41 (s, 9H).

### Step 4: tert-butyl 6-(5-hydroxypyrazine-2-carbonyl)-8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2-carboxylate

To a solution of 5-hydroxypyrazine-2-carboxylic acid (128 mg, 0.916 mmol) in DMF (7 mL) was added HATU (348 mg, 0.916 mmol) and DIPEA (296 mg, 2.29 mmol) and the mixture stirred at room temperature for 30 min. tert-butyl 8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2-carboxylate (350 mg, 0.763 mmol) was added and stirring continued for another 2 h. The solvent was removed and the residue obtained purified by R.P column to afford tert-butyl 6-(5-hydroxypyrazine-2-carbonyl)-8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2-carboxylate (310 mg, 70%) as a yellow solid. LCMS m/z=459.2 [M+H]⁺; ¹H NMR (400 MHz, Chloroform-d) δ 7.54-7.45 (m, 1H), 6.16 (d, J=2.4 Hz, 1H), 5.73 (q, J=7.2 Hz, 1H), 4.06-3.93 (m, 3H), 3.91-3.82 (m, 1H), 3.69 (q, J=9.2 Hz, 2H), 3.55-3.44 (m, 3H), 3.37-3.20 (m, 4H), 2.96-2.83 (m, 1H), 1.97 (d, J=7.2 Hz, 3H), 1.89-1.71 (m, 5H), 1.41 (s, 9H).

### Step 5: (5-hydroxypyrazin-2-yl)(8-(5-(1-(3-(tetrahydro-2H-pyran-4-yl)-1H-pyrazol-1-yl)ethyl)-1,2,4-oxadiazol-3-yl)-2,6-diazaspiro[3.4]octan-6-yl)methanone

To a solution of tert-butyl 6-(5-hydroxypyrazine-2-carbonyl)-8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2-carboxylate in DCM (2 mL) was added TFA (1 mL) and the reaction was stirred for 2 h. The solvent was removed under vacuum to afford crude (5-hydroxypyrazin-2-yl)(8-(5-(1-(3-(tetrahydro-2H-pyran-4-yl)-1H-pyrazol-1-yl)ethyl)-1,2,4-oxadiazol-3-yl)-2,6-diazaspiro[3.4]octan-6-yl)methanone (150 mg, quant.) as a yellow oil. LCMS m/z=481.3 [M+H]⁺.

### Step 6: (2-((R)-2,2-difluorocyclopropane-1-carbonyl)-8-(5-(1-(3-(tetrahydro-2H-pyran-4-yl)-1H-pyrazol-1-yl)ethyl)-1,2,4-oxadiazol-3-yl)-2,6-diazaspiro[3.4]octan-6-yl)(5-hydroxypyrazin-2-yl)methanone)

To a solution of (R)-2,2-difluorocyclopropane-1-carboxylic acid (25.0 mg, 0.205 mmol) in DCM (5 mL) was added HATU (78 mg, 0.205 mmol) and DIPEA (89 mg, 0.683 mmol) and the mixture stirred at room temperature for 30 min. (5-hydroxypyrazin-2-yl)(8-(5-(1-(3-(tetrahydro-2H-pyran-4-yl)-1H-pyrazol-1-yl)ethyl)-1,2,4-oxadiazol-3-yl)-2,6-diazaspiro[3.4]octan-6-yl)methanone (82 mg, 0.17 mmol) was added and stirring continued for another 2 h. The reaction was quenched with water (20 mL) and extracted with DCM (30 mL×3). The combined organic layers were washed with brine, dried over Na₂SO₄, filtered and concentrated. The residue was purified by prep-HPLC to afford (2-((R)-2,2-difluorocyclopropane-1-carbonyl)-8-(5-(1-(3-(tetrahydro-2H-pyran-4-yl)-1H-pyrazol-1-yl)ethyl)-1,2,4-oxadiazol-3-yl)-2,6-diazaspiro[3.4]octan-6-yl)(5-hydroxypyrazin-2-yl)methanone) (22 mg, 22%) as yellow solid. LCMS m/z=585.2 [M+H]⁺; ¹H NMR (400 MHz, Methanol-d₄) δ 8.08-7.97 (m, 2H), 7.80-7.65 (m, 1H), 6.22 (s, 1H), 5.95-5.82 (m, 1H), 4.60-3.81 (m, 11H), 3.51 (t, J=11.6 Hz, 2H), 2.92-2.79 (m, 1H), 2.63-2.40 (m, 1H), 2.00-1.88 (m, 4H), 1.86-1.66 (m, 5H).

Table 32: The compounds listed in Table 32 were synthesized from (5-hydroxypyrazin-2-yl)(8-(5-(1-(3-(tetrahydro-2H-pyran-4-yl)-1H-pyrazol-1-yl)ethyl)-1,2,4-oxadiazol-3-yl)-2,6-diazaspiro[3.4]octan-6-yl)methanone according to the procedures outlined for I-116 using the appropriate commercially available reagents and/or intermediates described elsewhere.

TABLE 32

| Example | | | |
|---|---|---|---|
| Number | Compound | ¹HNMR | LCMS |
| I-115 | 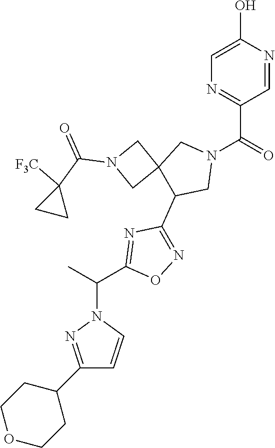 [SMILES: CC(c1nc(C2CN(C(=O)c3cnc(O)cn3)CC23CN(C(=O)C2(C(F)(F)F)CC2)C3)no1)n1ccc(C2CCOCC2)n1] | ¹H NMR (400 MHz, DMSO-d₆) δ 7.84 − 7.79 (m, 1H), 7.99 (s, 1H), 7.91 − 7.85 (m, 1H), 6.18 (d, J = 2.4 Hz, 1H), 6.10 − 5.93 (m, 1H), 4.50 − 3.73 (m, 11H), 3.43 − 3.34 (m, 3H), 2.84 − 2.69 (m, 1H), 1.90 − 1.79 (m, 3H), 1.78 − 1.69 (m, 2H), 1.64 − 1.51 (m, 2H), 1.30 − 0.98 (m, 4H). | m/z = 617.2 [M + H]⁺ |

### Synthesis of 4-((4-(8-(5-((3-cyclopentyl-1H-pyrazol-1-yl)methyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)benzoic acid (I-128)

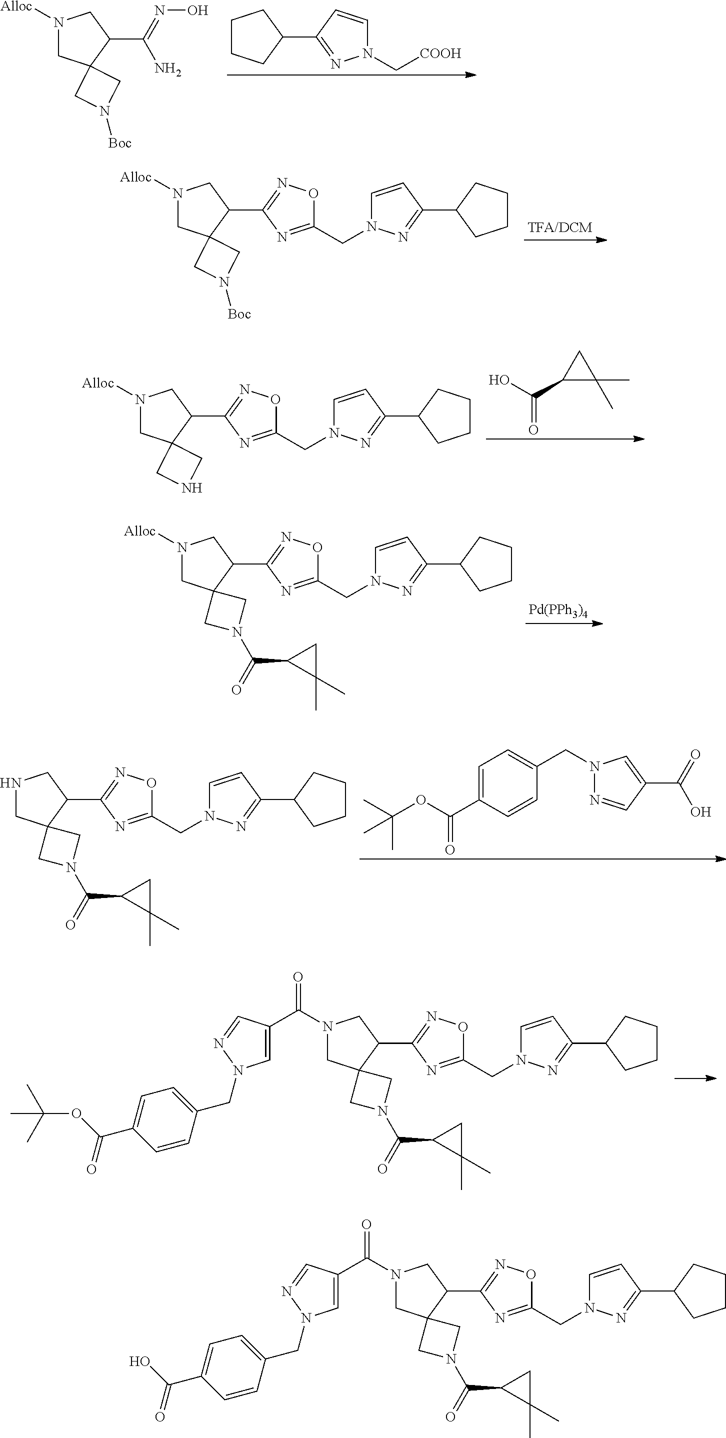

### Step 1: 6-allyl 2-(tert-butyl) 8-(5-((3-cyclopentyl-1H-pyrazol-1-yl)methyl)-1,2,4-oxadiazol-3-yl)-2,6-diazaspiro[3.4]octane-2,6-dicarboxylate

To a solution of 6-allyl 2-(tert-butyl) (Z)-8-(N′-hydroxycarbamimidoyl)-2,6-diazaspiro[3.4]octane-2,6-dicarboxylate (200 mg, 0.38 mmol) and 2-(3-cyclopentyl-1H-pyrazol-1-yl)acetic acid (132 mg, 0.68 mmol) in DCM (4 mL) was added HATU (215 mg, 0.56 mmol) and DIPEA (219 mg, 1.69 mmol). The mixture was stirred at room temperature for 2 h then was diluted with water (30 mL) and extracted with DCM (50 mL×2). The combined organic layers were washed with brine, dried over Na₂SO₄, filtered and concentrated. The residue was re-dissolved in DMF (5 mL) and DBU (69 mg, 0.45 mmol) was added. The reaction was heated at 90° C. for 2 h then was diluted with water (20 mL) and extracted with DCM (50 mL×2). The combined organic layers were washed with brine, dried over Na₂SO₄, filtered and concentrated. The residue obtained was purified by prep-TLC (eluent: DCM:MeOH=20:1) to afford 6-allyl 2-(tert-butyl) 8-(5-((3-cyclopentyl-1H-pyrazol-1-yl)methyl)-1,2,4-oxadiazol-3-yl)-2,6-diazaspiro[3.4]octane-2,6-dicarboxylate (100 mg, 52%) as a colorless oil. LCMS m/z=513.2 [M+H]⁺; ¹H NMR (400 MHz, DMSO-d₆) δ 7.74 (d, J=2.2 Hz, 1H), 6.14 (d, J=2.2 Hz, 1H), 5.97-5.86 (m, 1H), 5.69 (s, 2H), 5.34-5.24 (m, 1H), 5.18 (dd, J=10.6, 5.4 Hz, 1H), 4.52 (d, J=5.0 Hz, 2H), 3.96 (s, 1H), 3.82 (m, 2H), 3.73-3.55 (m, 7H), 2.97 (p, J=7.8 Hz, 1H), 1.91 (tq, J=7.0, 3.2 Hz, 2H), 1.68-1.52 (m, 6H), 1.35 (s, 9H).

### Step 2: allyl 8-(5-((3-cyclopentyl-1H-pyrazol-1-yl)methyl)-1,2,4-oxadiazol-3-yl)-2,6-diazaspiro [3.4]octane-6-carboxylate

To a solution of 6-allyl 2-(tert-butyl) 8-(5-((3-cyclopentyl-1H-pyrazol-1-yl)methyl)-1,2,4-oxadiazol-3-yl)-2,6-diazaspiro[3.4]octane-2,6-dicarboxylate (100 mg, 0.20 mmol) in DCM (2 mL) was added TFA (1 mL) and the reaction stirred at room temperature for 2 h. The solvent was removed under vacuum to afford allyl 8-(5-((3-cyclopentyl-1H-pyrazol-1-yl)methyl)-1,2,4-oxadiazol-3-yl)-2,6-diazaspiro[3.4]octane-6-carboxylate (80 mg, 100%) as a brown oil.

### Step 3: allyl 8-(5-((3-cyclopentyl-1H-pyrazol-1-yl)methyl)-1,2,4-oxadiazol-3-yl)-2-((S)-2,2-dimethylcyclopropane-1-carbonyl)-2,6-diazaspiro[3.4]octane-6-carboxylate

To a solution of (S)-2,2-dimethylcyclopropane-1-carboxylic acid (27 mg, 0.23 mmol) in DCM (2 mL) was added HATU (74 mg, 0.19 mmol) and DIPEA (75 mg, 0.58 mmol) and the mixture stirred at room temperature for 10 min. Allyl 8-(5-((3-cyclopentyl-1H-pyrazol-1-yl)methyl)-1,2,4-oxadiazol-3-yl)-2,6-diazaspiro[3.4]octane-6-carboxylate (80 mg, 0.19 mmol) was added and stirring continued for another 2 h. The mixture was diluted with water (20 mL) and extracted with DCM (30 mL×2). The combined organic layers were washed with brine, dried over Na₂SO₄, filtered and concentrated. The residue obtained was purified by prep-TLC (eluent: DCM:MeOH=25:1) to afford allyl 8-(5-((3-cyclopentyl-1H-pyrazol-1-yl)methyl)-1,2,4-oxadiazol-3-yl)-2-((S)-2,2-dimethylcyclopropane-1-carbonyl)-2,6-diazaspiro[3.4]octane-6-carboxylate (60 mg, 61%) as a colorless oil. ¹H NMR (400 MHz, DMSO-d₆) δ 7.74 (t, J=2.8 Hz, 1H), 6.14 (d, J=2.2 Hz, 1H), 5.93 (td, J=11.6, 5.4 Hz, 1H), 5.69 (t, J=4.2 Hz, 2H), 5.34-5.15 (m, 2H), 4.53 (d, J=5.0 Hz, 2H), 4.21-3.64 (m, 9H), 2.97 (p, J=8.0 Hz, 1H), 1.97-1.86 (m, 2H), 1.70-1.53 (m, 6H), 1.31 (m, 1H), 1.13-0.93 (m, 6H), 0.84 (q, J=4.6 Hz, 1H), 0.67 (m, 1H).

### Step 4: (8-(5-((3-cyclopentyl-1H-pyrazol-1-yl)methyl)-1,2,4-oxadiazol-3-yl)-2,6-diazaspiro[3.4]octan-2-yl)((S)-2,2-dimethylcyclopropyl)methanone

To a solution of allyl 8-(5-((3-cyclopentyl-1H-pyrazol-1-yl)methyl)-1,2,4-oxadiazol-3-yl)-2-((S)-2,2-dimethylcyclopropane-1-carbonyl)-2,6-diazaspiro[3.4]octane-6-carboxylate (400 mg, 0.79 mmol), Pd(PPh₃)₄ (91 mg, 0.08 mmol) and PPh₃ (52 mg, 0.20 mmol) in DCM (5 mL) was added pyrrolidine (67 mg, 0.94 mmol). The reaction was stirred at room temperature for 1 h then was diluted with water (20 mL) and extracted with DCM (50 mL×3). The combined organic layers were washed with brine, dried over Na₂SO₄, filtered and concentrated to afford (8-(5-((3-cyclopentyl-1H-pyrazol-1-yl)methyl)-1,2,4-oxadiazol-3-yl)-2,6-diazaspiro[3.4]octan-2-yl)((S)-2,2-dimethylcyclopropyl)methanone (550 mg, 100% crude) as a brown oil. LCMS m/z=425.2 [M+H]⁺.

### Step 5: tert-butyl 4-((4-(8-(5-((3-cyclopentyl-1H-pyrazol-1-yl)methyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)benzoate

To a solution of 1-(4-(tert-butoxycarbonyl)benzyl)-1H-pyrazole-4-carboxylic acid (300 mg, 0.99 mmol) in DCM (5 mL) was added HATU (378 mg, 0.99 mmol) and DIPEA (385 mg, 2.80 mmol) and the mixture stirred at room temperature for 10 min. (8-(5-((3-cyclopentyl-1H-pyrazol-1-yl)methyl)-1,2,4-oxadiazol-3-yl)-2,6-diazaspiro[3.4]octan-2-yl)((S)-2,2-dimethylcyclopropyl)methanone (506 mg, 1.19 mmol) was added and stirring continued for another 2 h. The mixture was diluted with water (30 mL) and extracted with DCM (50 mL×3). The combined organic layers were washed with brine, dried over Na₂SO₄, filtered and concentrated. The residue obtained was purified by column chromatography on silica gel (eluent: DCM:MeOH=60:1 to 30:1) to afford tert-butyl 4-((4-(8-(5-((3-cyclopentyl-1H-pyrazol-1-yl)methyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)benzoate (200 mg, 28%) as a colorless oil. LCMS m/z=709.2 [M+H]⁺; ¹H NMR (400 MHz, DMSO-d₆) δ 12.91 (s, 1H), 8.42 (d, J=14.8 Hz, 1H), 7.95-7.83 (m, 3H), 7.73 (s, 1H), 7.38-7.29 (m, 2H), 6.13 (s, 1H), 5.69 (d, J=4.6 Hz, 2H), 5.45 (d, J=4.0 Hz, 2H), 4.35-3.74 (m, 9H), 2.96 (m, 1H), 1.90 (s, 2H), 1.60 (m, 6H), 1.53 (s, 9H), 1.37-1.23 (m, 1H), 1.13-0.92 (m, 6H), 0.84 (s, 1H), 0.65 (m, 1H).

### Step 6: 4-((4-(8-(5-((3-cyclopentyl-1H-pyrazol-1-yl)methyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)benzoic acid

To a solution of tert-butyl 4-((4-(8-(5-((3-cyclopentyl-1H-pyrazol-1-yl)methyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)benzoate (150 mg, 0.21 mmol) in DCM (3 mL) was added TFA (3 mL). The reaction mixture was stirred at room temperature for 2 h then the solvent was removed under vacuum. The residue obtained was purified by prep-HPLC to afford 4-((4-(8-(5-((3-cyclopentyl-1H-pyrazol-1-yl)methyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)methyl)benzoic acid (30 mg, 22%) as a brown oil. LCMS m/z=653.3 [M+H]⁺; ¹H NMR (400 MHz, DMSO-d₆) δ 12.91 (s, 1H), 8.42 (d, J=14.8 Hz, 1H), 7.95-7.83 (m, 3H), 7.73 (s, 1H), 7.38-7.29 (m, 2H), 6.13 (s, 1H), 5.69 (d, J=4.6 Hz, 2H), 5.45 (d, J=4.0 Hz, 2H), 4.35-3.74 (m, 9H), 2.96 (m, 1H), 1.90 (s, 2H), 1.69-1.53 (m, 6H), 1.37-1.23 (m, 1H), 1.13-0.92 (m, 6H), 0.84 (s, 1H), 0.71-0.61 (m, 1H).

Table 33: The compounds listed in Table 33 were synthesized from (2-((S)-2,2-dimethylcyclopropane-1-carbonyl)-8-ethynyl-2,6-diazaspiro[3.4]octan-6-yl)(thiazol-5-yl)methanone according to the procedures outlined for I-51 using the appropriate commercially available reagents and/or intermediates described elsewhere.

TABLE 33

| Example | | | |
|---|---|---|---|
| Number | Compound | ¹HNMR | LCMS |
| I-202 | 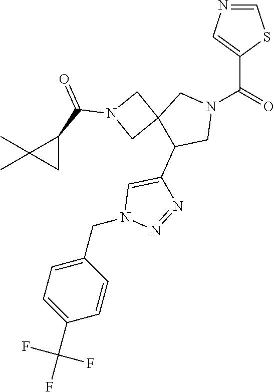 [SMILES: CC1(C)C[C@@H]1C(=O)N1CC2(CN(C(=O)c3cncs3)CC2c2cn(Cc3ccc(C(F)(F)F)cc3)nn2)C1] | ¹H NMR (400 MHz, Chloroform-d) δ 8.96 − 8.87 (m, 1H), 8.32 − 8.22 (m, 1H), 7.71 − 7.61 (m, 2H), 7.49 − 7.31 (m, 3H), 5.59 (s, 2H), 4.47 − 4.15 (m, 3H), 4.10 − 3.95 (m, 3H), 3.92 − 3.59 (m, 3H), 1.27 (s, 1H), 1.08 (s, 6H), 0.89 (s, 1H), 0.72 (s, 1H). | m/z = 545.2 [M + H]⁺ |
| I-195 | 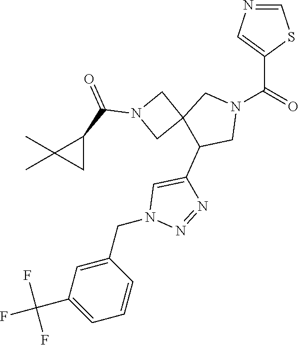 [SMILES: CC1(C)C[C@@H]1C(=O)N1CC2(CN(C(=O)c3cncs3)CC2c2cn(Cc3cccc(C(F)(F)F)c3)nn2)C1] | ¹H NMR (MeOD, 400 MHz) δ 9.17 (s, 1H), 8.41 (d, J = 9.6 Hz, 1H), 8.15 (d, J = 7.2 Hz, 1H), 7.72 − 7.47 (m, 4H), 5.71 (d, J = 8.6 Hz, 2H), 4.42 − 3.77 (m, 9H), 1.39 − 1.24 (m, 1H), 1.16 (s, 1H), 1.13 − 1.02 (m, 3H), 1.00 − 0.92 (m, 1H), 0.88 (d, J = 4.2 Hz, 1H), 0.77 − 0.63 (m, 2H). | m/z = 545.3 [M + H]⁺ |
| I-193 | 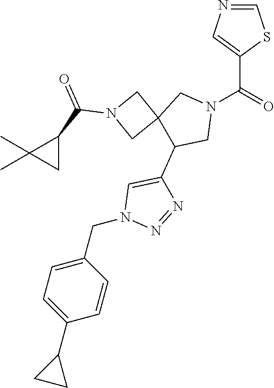 [SMILES: CC1(C)C[C@@H]1C(=O)N1CC2(CN(C(=O)c3cncs3)CC2c2cn(Cc3ccc(C4CC4)cc3)nn2)C1] | ¹H NMR (400 MHz, DMSO-d₆) δ 9.27 − 9.23 (m, 1H), 8.43 − 8.39 (m, 1H), 8.24 − 8.19 (m, 1H), 7.22 − 7.09 (m, 2H), 7.06 − 7.02 (m, 2H), 5.55 − 5.48 (m, 2H), 4.26 − 3.61 (m, 9H), 1.91 − 1.85 (m, 1H), 1.13 − 0.98 (m, 4H), 0.97 − 0.90 (m, 3H), 0.86 − 0.74 (m, 2H), 0.68 − 0.55 (m, 4H). | m/z = 517.3 [M + H]⁺ |
| I-194 | 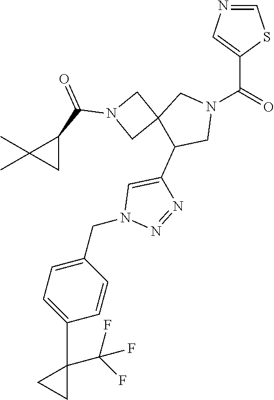 [SMILES: CC1(C)C[C@@H]1C(=O)N1CC2(CN(C(=O)c3cncs3)CC2c2cn(Cc3ccc(C4(C(F)(F)F)CC4)cc3)nn2)C1] | ¹H NMR (400 MHz, Chloroform-d) δ 8.91 (d, J = 8.1 Hz, 1H), 8.32 − 8.22 (m, 1H), 7.50 − 7.35 (m, 3H), 7.25 − 7.17 (m, 2H), 5.64 − 5.42 (m, 2H), 4.37 − 3.63 (m, 9H), 1.39 − 1.34 (m, 2H), 1.18 − 0.98 (m, 10H), 0.76 − 0.64 (m, 1H). | m/z = 585.3 [M + H]⁺ |

### Synthesis of 1-amino-3-(3-chloro-4-(trifluoromethyl)phenyl)propan-2-ol

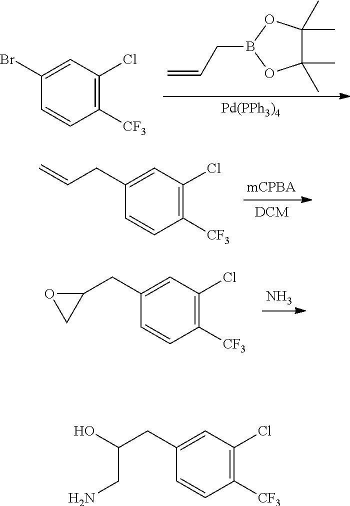

2-(3-chloro-4-(trifluoromethyl)benzyl)oxirane was synthesized from 4-bromo-2-chloro-1-(trifluoromethyl)benzene according to the procedures outlined for 2-(4-chloro-3-(trifluoromethyl)benzyl)oxirane using the appropriate commercially available reagents and/or intermediates described elsewhere. ¹H NMR (400 MHz, CD₃OD) δ 7.70 (d, J=8.1 Hz, 1H), 7.55 (s, 1H), 7.40 (d, J=8.1 Hz, 1H), 3.18 (ddt, J=6.7, 4.1, 2.1 Hz, 1H), 3.03 (dd, J=14.7, 4.3 Hz, 1H), 2.86-2.78 (m, 2H), 2.57 (dd, J=4.9, 2.6 Hz, 1H).

1-amino-3-(3-chloro-4-(trifluoromethyl)phenyl)propan-2-ol was synthesized from 2-(3-chloro-4-(trifluoromethyl)benzyl)oxirane according to the procedures outlined for 1-amino-3-(4-chloro-3-(trifluoromethyl)phenyl)propan-2-ol using the appropriate commercially available reagents and/or intermediates described elsewhere. LCMS m/z=254.0 [M+H]⁺.

### Synthesis of 2-(4-bromophenyl)-2,2-difluoroacetohydrazide

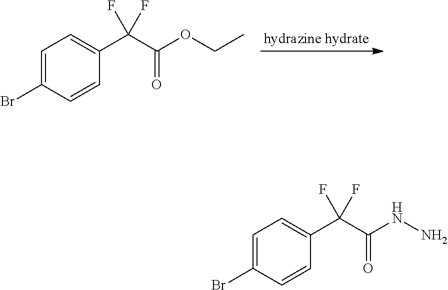

### Step 1: 2-(4-bromophenyl)-2,2-difluoroacetohydrazide

To a solution of ethyl 2-(4-bromophenyl)-2,2-difluoroacetate (280 mg, 1.0 mmol) in MeOH (3 mL) was added 98% hydrazine hydrate (0.5 mL). The mixture was stirred at room temperature for 4 h then was diluted with water (10 mL) and extracted with DCM/MeOH=5/1 (v/v, 50 mL×3). The combined organic layers was washed with brine, dried over Na₂SO₄, filtered and concentrated. The residue was purified by prep-TLC (eluent: DCM:MeOH=20:1) afford 2-(4-bromophenyl)-2,2-difluoroacetohydrazide (210 mg, 79%) as a white solid. ¹H NMR (400 MHz, DMSO-d₆) δ 10.29 (s, 1H), 7.74 (d, J=8.2 Hz, 2H), 7.52 (d, J=8.2 Hz, 2H), 4.55 (s, 2H).

### Synthesis of 2-(5-(methoxycarbonyl)-3-methyl-1H-pyrazol-1-yl)propanoic acid

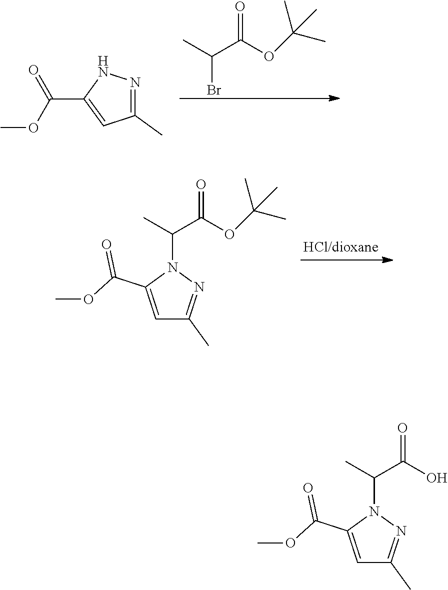

### Step 1: methyl 1-(1-(tert-butoxy)-1-oxopropan-2-yl)-3-methyl-1H-pyrazole-5-carboxylate

To a solution of methyl 3-methyl-1H-pyrazole-5-carboxylate (500 mg, 3.6 mmol) in DMF (5.0 mL) was added tert-butyl 2-bromopropanoate (821 mg, 3.9 mmol) and Cs₂CO₃ (2.3 g, 7.1 mmol). The resulting mixture was stirred at room temperature overnight then was diluted with water (30 mL) and extracted with EtOAc (50 mL×2). The combined organic layers was washed with brine, dried over Na₂SO₄, filtered and concentrated. The residue was purified by RP-column to afford methyl 1-(1-(tert-butoxy)-1-oxopropan-2-yl)-3-methyl-1H-pyrazole-5-carboxylate (680 mg, 71%) as a white solid. LCMS m/z=269.1 [M+H]⁺; ¹H NMR (400 MHz, CD₃OD) δ 6.57 (t, J=0.8 Hz, 1H), 5.12 (q, J=7.2 Hz, 1H), 3.86 (s, 3H), 2.31 (d, J=0.8 Hz, 3H), 1.75 (d, J=7.2 Hz, 3H), 1.41 (s, 9H).

### Step 2: 2-(5-(methoxycarbonyl)-3-methyl-1H-pyrazol-1-yl)propanoic acid

To a solution of methyl 1-(1-(tert-butoxy)-1-oxopropan-2-yl)-3-methyl-1H-pyrazole-5-carboxylate (300 mg, 1.1 mmol) in dioxane (1.0 mL) was added HCl/dioxane (2M solution 3 mL). The mixture was stirred at room temperature for 2 h then concentrated under vacuum to afford 2-(5-(methoxycarbonyl)-3-methyl-1H-pyrazol-1-yl)propanoic acid (237 mg, quant) as a yellow oil. LCMS m/z=213.1 [M+H]⁺.

### Synthesis of 2-nitro-5-phenoxyphenol

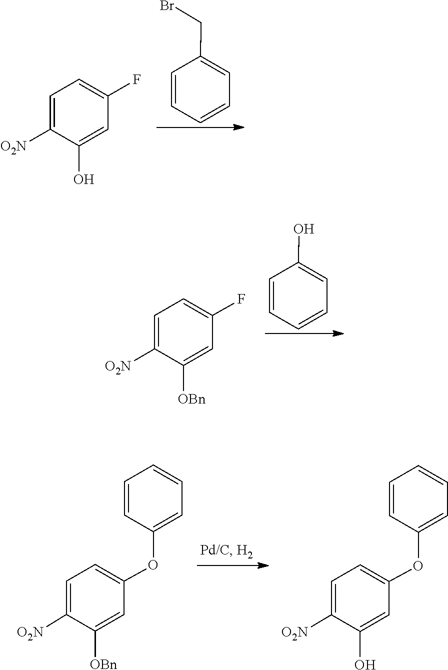

### Step 1: 2-(benzyloxy)-4-fluoro-1-nitrobenzene

To a solution of 5-fluoro-2-nitrophenol (1.0 g, 6.37 mmol) in DMF (10 mL) under a N₂ atmosphere was added potassium carbonate (1.06 g, 7.64 mmol) and benzyl bromide (0.83 mL, 7.00 mmol). The reaction mixture was heated at 60° C. for 2 h then was coiled to room temperature, diluted with water (30 mL) and extracted with EtOAc (50 mL×2). The combined organic layers were washed with brine, dried over Na₂SO₄, filtered and concentrated to afford 2-(benzyloxy)-4-fluoro-1-nitrobenzene (0.8 g, 50%) as a yellow solid. ¹H NMR (400 MHz, CDCl₃) δ 7.97 (dd, J=9.1, 6.0 Hz, 1H), 7.48-7.33 (m, 5H), 6.83 (dd, J=10.2, 2.5 Hz, 1H), 6.78-6.70 (m, 1H), 5.23 (s, 2H).

### Step 2: 2-(benzyloxy)-1-nitro-4-phenoxybenzene

To a solution of 2-(benzyloxy)-4-fluoro-1-nitrobenzene (0.5 g, 2.02 mmol) in DMF (5 mL) under a N₂ atmosphere was added potassium carbonate (0.28 g, 2.02 mmol) and phenol (0.19 g, 2.02 mmol). The reaction was heated at 100° C. overnight then was diluted with water (20 mL) and extracted with EtOAc (30 mL×3). The combined organic layers were washed with brine, dried over Na₂SO₄, filtered and concentrated to afford 2-(benzyloxy)-1-nitro-4-phenoxybenzene (0.8 g, 62%) as a yellow solid. ¹H NMR (400 MHz, CDCl₃) δ 7.95 (d, J=9.2 Hz, 1H), 7.46-7.31 (m, 8H), 7.26-7.21 (m, 1H), 7.07-6.80 (m, 3H), 6.66 (d, J=2.4 Hz, 1H), 6.52 (dd, J=9.0, 2.4 Hz, 1H), 5.15 (s, 2H).

### Step 3: 2-nitro-5-phenoxyphenol

To a solution of 2-(benzyloxy)-1-nitro-4-phenoxybenzene (0.4 g, 1.73 mmol) in ethanol (4.0 mL) under a H₂ atmosphere was added Pd/C (10%, 400 mg). The reaction mixture was heated at 40° C. overnight. The catalyst was removed by filtration through celite and the filtrate concentrated to afford 2-amino-5-phenoxypheno (200 mg, 57%) which was used without further purification. LCMS m/z=202.1 [M+H]⁺; ¹H NMR (400 MHz, CDCl₃) δ 7.35-7.28 (m, 11H), 7.26-7.21 (m, 11H), 7.04 (t, J=7.3 Hz, 11H), 6.95 (d, J=8.0 Hz, 2H), 6.76 (d, J=8.2 Hz, 1H), 6.55-6.43 (m, 2H).

### Synthesis of 2-amino-4-phenoxyphenol

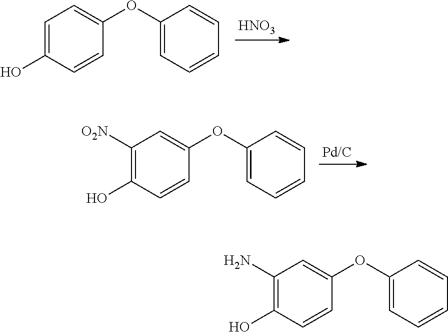

### Step 1: 2-nitro-4-phenoxyphenol

To a solution of 4-phenoxyphenol (2 g, 10.7 mmol) in AcOH (50 mL) was added 70% HNO₃ (720 mg, 10.7 mmol) dropwise. The mixture was stirred at room temperature for 15 min then was poured into ice water (100 mL) and extracted with EtOAc (150 mL×2). The combined organic layers were washed with brine, dried over Na₂SO₄, filtered and concentrated. The residue obtained was purified by column chromatography on silica gel (eluent: Pet. Ether:EtOAc=20:1-5:1) to afford 2-nitro-4-phenoxyphenol (1.3 g, 52%). LCMS m/z=459.1 [M+H]⁺.

### Step 2: 2-amino-4-phenoxyphenol

To a solution of 2-nitro-4-phenoxyphenol (1.3 g, 5.6 mmol) in ethanol (8.0 mL) was added 10% Pd/C (150 mg) an the reaction stirred under a H₂ atmosphere overnight. The catalyst was removed by filtration through celite and the filtrate concentrated to afford 2-amino-4-phenoxypheno (1.0 g, 91%) which was used without further purification. LCMS m/z=202.1 [M+H]⁺.

### Synthesis of 2,2-difluoro-2-(4-(tetrahydro-2H-pyran-4-yl)phenyl)acetohydrazide

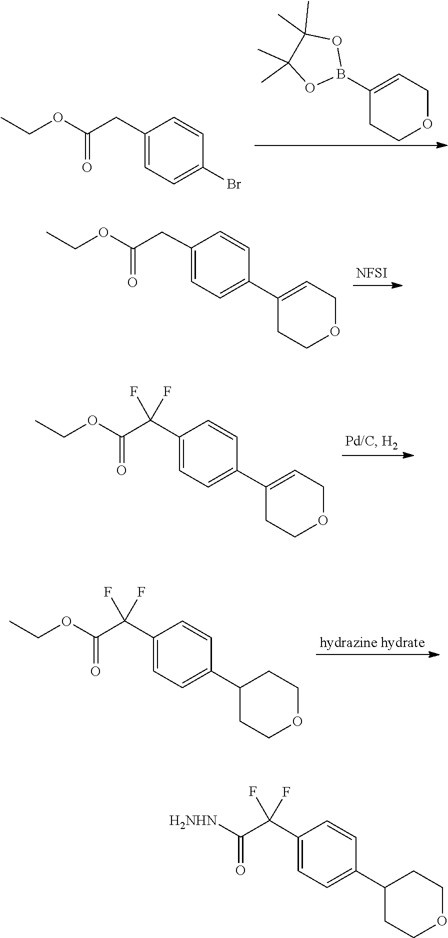

### Step 1: ethyl 2-(4-(3,6-dihydro-2H-pyran-4-yl)phenyl)acetate

To a solution of ethyl 2-(4-bromophenyl)acetate (5.0 g, 20.57 mmol), 2-(3,6-dihydro-2H-pyran-4-yl)-4,4,5,5-tetramethyl-1,3,2-dioxaborolane (5.2 g, 24.68 mmol) and sodium carbonate (6.54 g, 61.70 mmol) in a mixture of DME and H₂O (50 mL and 10 mL) was added Pd(PPh₃)₄ (1.19 g, 61.70 mmol). The reaction mixture was heated at reflux for 4 hours then was diluted with water (80 mL) and extracted with EtOAc (100 mL×2). The combined organic layers were washed with brine, dried over Na₂SO₄, filtered and concentrated. The residue was purified by column chromatography on silica gel (eluent: Pet. Ether:EtOAc=30:1) to afford ethyl 2-(4-(3,6-dihydro-2H-pyran-4-yl)phenyl)acetate (2.9 g, 57%) as a yellow solid. ¹H NMR (400 MHz, CDCl₃) δ 7.39-7.29 (m, 2H), 7.30-7.16 (m, 2H), 6.16-6.08 (m, 1H), 4.36-4.27 (m, 2H), 4.14 (q, J=7.2 Hz, 2H), 3.92 (t, J=5.5 Hz, 2H), 1.29-1.23 (m, 3H).

### Step 2: ethyl 2-(4-(3,6-dihydro-2H-pyran-4-yl)phenyl)-2,2-difluoroacetate

To a solution of ethyl 2-(4-(3,6-dihydro-2H-pyran-4-yl)phenyl)acetate (4.9 g, 19.89 mmol) in anhydrous THF (50 mL) at −78° C. under a N₂ atmosphere was added LiHDMS (1.0 M in THF, 43.76 mmol) dropwise. The reaction mixture was stirred at −78° C. for 30 min then a solution of NFSI (13.8 g, 43.76 mmol) in anhydrous THF (50 mL) was added. The reaction was allowed to warm to room temperature and stirred for 1 h. The reaction was quenched with 1.0 M HCl (100 mL) and extracted with EtOAc (150 mL×2). The combined organic layers were washed with brine, dried over Na₂SO₄, filtered and concentrated. The residue was purified by column chromatography on silica gel (eluent: Pet. Ether:EtOAc=30:1) to afford ethyl 2-(4-(3,6-dihydro-2H-pyran-4-yl)phenyl)-2,2-difluoroacetate (2.9 g, 52%) as a yellow solid. ¹H NMR (400 MHz, CD₃OD) δ 7.55 (s, 4H), 6.35-6.26 (m, 1H), 4.35-4.24 (m, 4H), 3.92 (t, J=5.5 Hz, 2H), 2.56-2.49 (m, 2H), 1.27 (t, J=7.1 Hz, 3H).

### Step 3: ethyl 2,2-difluoro-2-(4-(tetrahydro-2H-pyran-4-yl)phenyl)acetate

To a solution of ethyl 2-(4-(3,6-dihydro-2H-pyran-4-yl)phenyl)-2,2-difluoroacetate (2.9 g, 2.92 mmol) in methanol (30 mL) was added 10% Pd/C (500 mg) and the reaction was stirred under a H₂ atmosphere overnight. The catalyst was removed by filtration through celite and the filtrate was concentrated to afford ethyl 2,2-difluoro-2-(4-(tetrahydro-2H-pyran-4-yl)phenyl)acetate (2.8 g, 96%) as a white solid. ¹H NMR (400 MHz, CD₃OD) δ 7.52 (d, J=8.0 Hz, 2H), 7.39 (d, J=8.0 Hz, 2H), 4.28 (q, J=7.2 Hz, 2H), 4.09-3.98 (m, 2H), 3.62-3.51 (m, 2H), 2.92-2.80 (m, 1H), 1.83-1.71 (m, 4H), 1.27 (t, J=7.2 Hz, 3H).

### Step 4: 2,2-difluoro-2-(4-(tetrahydro-2H-pyran-4-yl)phenyl)acetohydrazide

To a solution of ethyl 2,2-difluoro-2-(4-(tetrahydro-2H-pyran-4-yl)phenyl)acetate (2.8 g, 9.86 mmol) in methanol (30 mL) was added 98% hydrazine hydrate (2.5 g, 39.44 mmol). The mixture was stirred at room temperature for 30 min then was diluted with water (30 mL) and extracted with DCM/MeOH=5/1 (60 mL×3). The combined organic layers were washed with brine, dried over Na₂SO₄, filtered and concentrated to afford 2,2-difluoro-2-(4-(tetrahydro-2H-pyran-4-yl)phenyl)acetohydrazide (2.2 g, 83%) as a white solid. LCMS m/z=271.1 [M+H]⁺; ¹H NMR (400 MHz, CD₃OD) δ 7.55 (d, J=8.1 Hz, 2H), 7.37 (d, J=8.1 Hz, 2H), 4.08-4.01 (m, 2H), 3.64-3.50 (m, 2H), 2.92-2.81 (m, 1H), 1.86-1.70 (m, 4H).

### Synthesis of 1-(4-cyanobenzyl)-1H-pyrazole-4-carboxylic acid

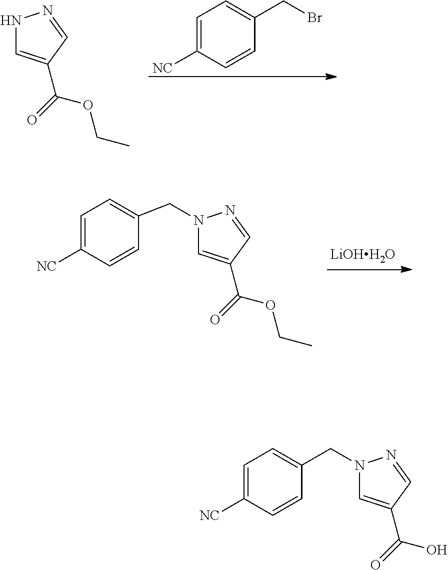

### Step 1: ethyl 1-(4-cyanobenzyl)-1H-pyrazole-4-carboxylate

To a solution of ethyl 1H-pyrazole-4-carboxylate (1 g, 7.14 mmol) in DMF (10 mL) was added 4-(bromomethyl)benzonitrile (1.47 g, 7.50 mmol) and K₂CO₃ (2.96 g, 21.41 mmol) and the reaction stirred at room temperature overnight. The mixture was diluted with water (60 mL) and extracted with DCM (80 mL×2). The combined organic layers was washed with brine, dried over Na₂SO₄, filtered and concentrated to afford ethyl 1-(4-cyanobenzyl)-1H-pyrazole-4-carboxylate (1.8 g, quant) as a white solid. LCMS m/z=256.1 [M+H]⁺; ¹H NMR (400 MHz, DMSO-d₆) δ 8.52 (s, 1H), 7.90 (s, 1H), 7.82 (d, 2H), 7.40 (d, J=8.0 Hz, 2H), 5.49 (s, 2H), 4.21 (q, J=7.2 Hz, 2H), 1.26 (t, J=7.2 Hz, 3H).

### Step 2: 1-(4-cyanobenzyl)-1H-pyrazole-4-carboxylic acid

To a solution of ethyl 1-(4-cyanobenzyl)-1H-pyrazole-4-carboxylate (200 mg, 0.78 mmol) in a mixture of MeOH and water (1.6 mL/0.4 mL) was added lithium hydroxide monohydrate (66 mg, 1.57 mmol). The reaction was stirred at room temperature for 3 h then was diluted with water (20 mL). The aqueous phase was adjusted to pH˜1 with 1N HCl, then extracted with EtOAc (50 mL×2). The combined organic layers were washed with brine, dried over Na₂SO₄, filtered and concentrated to afford 1-(4-cyanobenzyl)-1H-pyrazole-4-carboxylic acid (120 mg, 75%) as a white solid. LCMS m/z=228.2 [M+H]⁺; ¹H NMR (400 MHz, CD₃OD) δ 8.31-8.25 (m, 1H), 7.93-7.90 (m, 1H), 7.72 (d, 2H), 7.40 (d, 2H), 5.47 (s, 2H).

### Synthesis of 1-(3,5-difluoro-4-(methoxycarbonyl)benzyl)-1H-pyrazole-4-carboxylic acid

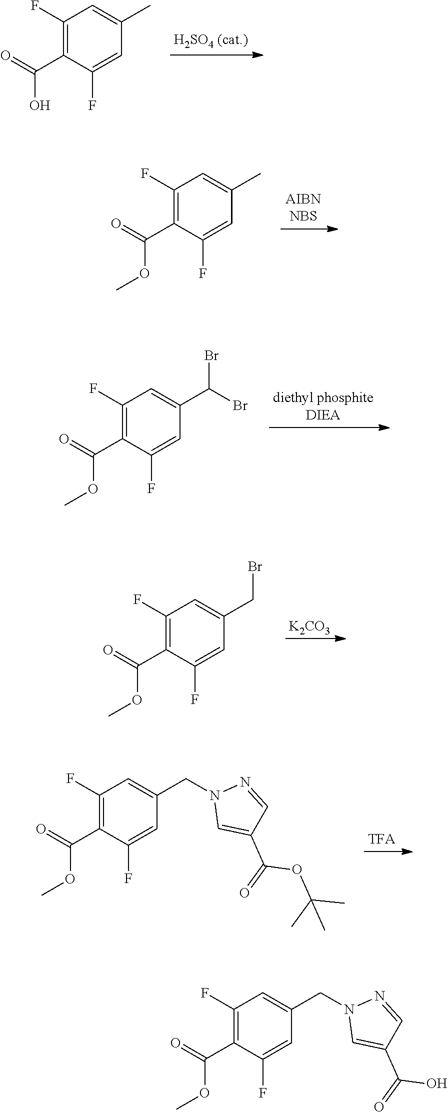

### Step 1: methyl 2,6-difluoro-4-methylbenzoate

To a solution of 2,6-difluoro-4-methylbenzoic acid (900 mg, 5.23 mmol) in MeOH (5 mL) was added H₂SO₄ (0.1 mL). The reaction mixture was heated at 70° C. for 50 hours then was diluted with water (50 mL), extracted with EtOAc (70 mL×3). The combined organic layers was washed with brine, dried over Na₂SO₄, filtered and concentrated. The residue was purified by column chromatography on silica gel (eluent: Pet. Ether:EtOAc=10:1) to afford methyl 2,6-difluoro-4-methylbenzoate (916 mg, 94%) as a colorless oil. ¹HNMR (400 MHz, Chloroform-d) δ 6.76 (d, J=9.6 Hz, 2H), 3.93 (s, 3H), 2.37 (s, 3H); ¹⁹F NMR (376 MHz, Chloroform-d) δ −111.05.

### Step 2: methyl 4-(dibromomethyl)-2,6-difluorobenzoate

To a solution of 2,6-difluoro-4-methylbenzoate (65 mg, 0.35 mmol) in CCl₄ (2 mL) at room temperature was added AIBN (23 mg, 0.14 mmol) and NBS (137 mg, 0.768 mmol). The reaction mixture was heated at reflux for 6 h then was diluted with water (10 mL) and extracted with DCM (20 mL×3). The combined organic layers were washed with brine, dried over Na₂SO₄, filtered and concentrated. The residue was purified by prep-TLC (eluent: Pet. Ether:EtOAc=25:1 v/v) to afford methyl 4-(dibromomethyl)-2,6-difluorobenzoate (30 mg, 25%) as a colorless oil. ¹H NMR (400 MHz, Chloroform-d) δ 7.18 (d, J=8.4 Hz, 2H), 6.51 (s, 1H), 3.96 (s, 3H); ¹⁹F NMR (376 MHz, Chloroform-d) δ −112.55.

### Step 3: methyl 4-(bromomethyl)-2,6-difluorobenzoate

To a solution of methyl 4-(dibromomethyl)-2,6-difluorobenzoate (500 mg, 1.45 mmol) in ACN (10 mL) was added DIEA (282 mg, 2.18 mmol) and diethyl phosphite (302 mg, 2.18 mmol). The reaction was stirred at room temperature overnight then was diluted with water (20 mL) and extracted with EtOAc (50 mL×2). The combined organic layers was dried over Na₂SO₄, filtered and concentrated. The residue was purified by prep-TLC (eluent: Pet. Ether/EtOAc=25/1, v/v) to afford methyl 4-(bromomethyl)-2,6-difluorobenzoate (370 mg, 96%) as a colorless oil. ¹H NMR (400 MHz, Chloroform-d) δ 7.00 (d, J=8.8 Hz, 2H), 4.39 (s, 2H), 3.95 (s, 3H). ¹⁹F NMR (376 MHz, Chloroform-d) δ −109.04.

### Step 4: tert-butyl 1-(3,5-difluoro-4-(methoxycarbonyl)benzyl)-1H-pyrazole-4-carboxylate

To a solution of tert-butyl 1H-pyrazole-4-carboxylate (381 mg, 2.26 mmol) in ACN (10 mL) was added K₂CO₃ (522 mg, 3.77 mmol) and methyl 4-(bromomethyl)-2,6-difluorobenzoate (500 mg, 1.89 mmol). The reaction was heated at 80° C. for 2 h then was filtered through celite and concentrated under reduced pressure. The residue obtained was purified by column chromatography on silica gel (eluent: Pet. Ether:EtOAc=3:1) to afford tert-butyl 1-(3,5-difluoro-4-(methoxycarbonyl)benzyl)-1H-pyrazole-4-carboxylate (410 mg, 62%) as a colorless oil. LCMS m/z=353.1 [M+H]⁻; ¹H NMR (400 MHz, Chloroform-d) δ 7.90 (s, 1H), 7.85 (s, 1H), 6.77 (d, J=8.8 Hz, 2H), 5.29 (s, 2H), 3.94 (s, 3H), 1.55 (s, 9H); ¹⁹F NMR (376 MHz, Chloroform-d) δ −108.39.

### Step 5: 1-(3,5-difluoro-4-(methoxycarbonyl)benzyl)-1H-pyrazole-4-carboxylic acid

To a solution of tert-butyl 1-(3,5-difluoro-4-(methoxycarbonyl)benzyl)-1H-pyrazole-4-carboxylate (200 mg, 0.57 mmol) in DCM (5 mL) was added TFA (2 mL) and the reaction stirred at room temperature for 2 hours. The solvent was removed under vacuum to afford 1-(3,5-difluoro-4-(methoxycarbonyl)benzyl)-1H-pyrazole-4-carboxylic acid (170 mg, quant.) which was used directly in the next step. LCMS m/z=297.0 [M+H]⁺; ¹H NMR (400 MHz, DMSO-d₆) δ 8.50 (s, 1H), 7.93 (s, 1H), 7.16 (d, J=9.4 Hz, 2H), 5.51 (s, 2H), 3.93 (s, 4H); ¹⁹F NMR (376 MHz, DMSO-d₆) δ −110.60.

### Synthesis of 1-((6-(methoxycarbonyl)pyridin-3-yl)methyl)-1H-pyrazole-4-carboxylic acid

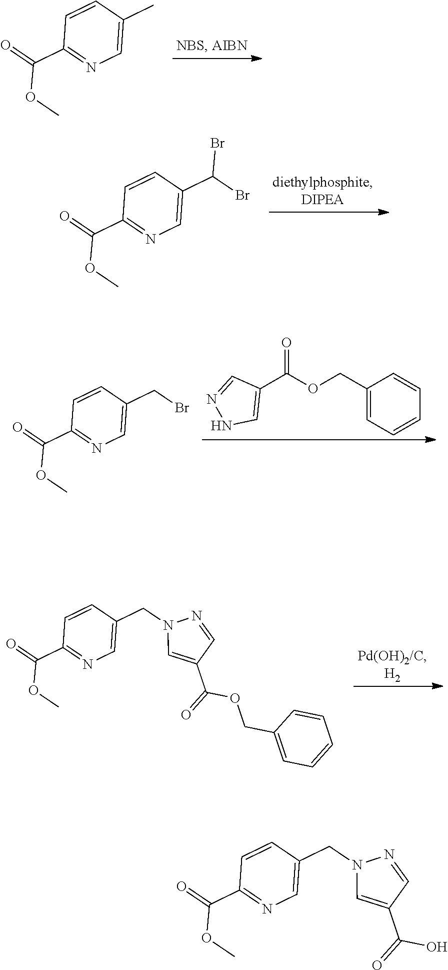

### Step 1: methyl 5-(dibromomethyl)picolinate

To a solution of methyl 5-methylpicolinate (1.0 g, 6.6 mmol) in CCl₄ (20 mL) was added AIBN (0.43 g, 2.64 mmol) and NBS (3.5 g, 19.7 mmol) and the reaction heated at reflux for 5 h. The mixture was diluted with water (30 mL) and extracted with DCM (50 mL×2). The combined organic layers were washed with brine, dried over Na₂SO₄, filtered and concentrated. The residue was purified by column chromatography on silica gel (eluent: Pet. Ether/EtOAc=20/1) to afford methyl 5-(dibromomethyl)picolinate (970 mg, 47%) as a white solid. ¹H NMR (400 MHz, Chloroform-d) δ 8.83 (d, J=2.2 Hz, 1H), 8.22-8.09 (m, 2H), 6.69 (s, 1H), 4.03 (s, 3H).

### Step 2: methyl 5-(bromomethyl)picolinate

To a solution of methyl 5-(dibromomethyl)picolinate (0.5 g, 1.62 mmol) in MeCN (10 mL) was added DIPEA (0.31 g, 2.43 mmol) and diethylphosphite (0.34 g, 2.43 mmol). The reaction was stirred at room temperature for 14 hours then was diluted with water (30 mL) and extracted with EtOAc (50 mL×2). The combined organic layers were washed with brine, dried over Na₂SO₄, filtered and concentrated. The residue was purified by column chromatography on silica gel (eluent: Pet. Ether/EtOAc=3/1) to afford afford methyl 5-(bromomethyl)picolinate (260 mg, 70%) as a white solid. ¹H NMR (400 MHz, Chloroform-d) δ 8.74 (s, 1H), 8.13 (d, J=8.1 Hz, 1H), 7.88 (dd, J=8.1, 2.2 Hz, 1H), 4.51 (s, 2H), 4.01 (s, 3H).

### Step 3: methyl 5-((4-((benzyloxy)carbonyl)-1H-pyrazol-1-yl)methyl)picolinate

To a solution of methyl 5-(bromomethyl)picolinate (120 mg, 0.52 mmol) in MeCN (5.0 mL) was added benzyl 1H-pyrazole-4-carboxylate (126 mg, 0.63 mmol) and K₂CO₃ (144 mg, 1.04 mmol). The reaction was stirred at room temperature for 14 h then was diluted with water (40 mL) and extracted with EtOAc (50 mL×2). The combined organic layers were washed with brine, dried over Na₂SO₄, filtered and concentrated. The residue was washed with diethyl ether (20 mL) to afford methyl 5-((4-((benzyloxy)carbonyl)-1H-pyrazol-1-yl)methyl)picolinate (118 mg, 65%) as a white solid. LCMS m/z=351.9 [M+H]⁺; ¹H NMR (400 MHz, Chloroform-d) δ 8.67 (d, J=2.2 Hz, 1H), 8.12 (d, J=8.1 Hz, 1H), 7.98 (d, J=13.1 Hz, 2H), 7.66 (dd, J=8.1, 2.2 Hz, 1H), 7.46-7.28 (m, 5H), 5.40 (s, 2H), 5.27 (s, 2H), 4.01 (s, 3H).

### Step 4: 1-((6-(methoxycarbonyl)pyridin-3-yl)methyl)-1H-pyrazole-4-carboxylic acid

To a solution of methyl 5-((4-((benzyloxy)carbonyl)-1H-pyrazol-1-yl)methyl)picolinate (200 mg, 0.57 mmol) in a mixture of MeOH and DCM (5.0 mL/1.0 mL) was added 10% Pd(OH)₂ on carbon (60 mg). The reaction mixture was stirred under a H₂ atmosphere for 3 h then the catalyst was removed by filtration through celite and the filtrate concentrated to afford 1-((6-(methoxycarbonyl)pyridin-3-yl)methyl)-1H-pyrazole-4-carboxylic acid (140 mg, 94%) as a white solid. LCMS m/z=261.7 [M+H]⁺; ¹H NMR (400 MHz, DMSO-d6) δ 8.66 (d, J=2.1 Hz, 1H), 8.45 (s, 1H), 8.04 (d, J=8.0 Hz, 1H), 7.85 (s, 1H), 7.80 (dd, J=8.1, 2.2 Hz, 1H), 5.52 (s, 2H), 3.87 (s, 3H).

### Synthesis of 1-((2,2-dimethyl-4-oxo-4H-benzo[d][1,3]dioxin-7-yl)methyl)-1H-pyrazole-4-carboxylic acid

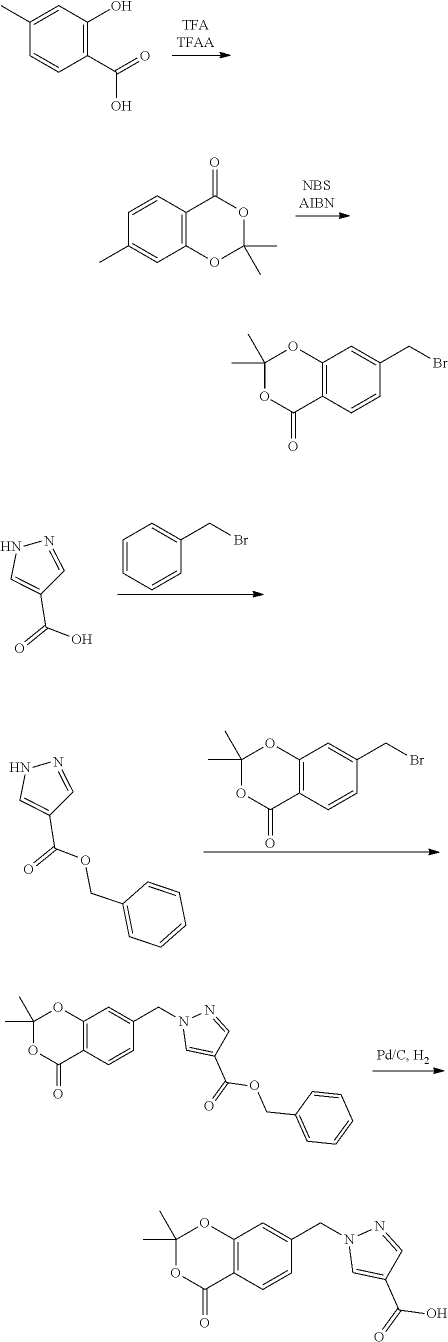

### Step 1: 2,2,7-trimethyl-4H-benzo[d][1,3]dioxin-4-one

To a solution of 2-hydroxy-4-methylbenzoic acid (5 g, 32.9 mmol) in acetone (6 mL) at 0° C. was added TFA (25 mL) and the mixture stirred for 5 min. TFAA (15 mL) was added and stirring continued for 3 days. The solvent was removed under reduced pressure and the residue obtained purified by column chromatography on silica gel (eluent: DCM) to afford 2,2,7-trimethyl-4H-benzo[d][1,3]dioxin-4-one (2.7 g, 43%) as yellow oil. LCMS: m/z=192.7 [M+H]⁺; ¹H NMR (400 MHz, CDCl₃) δ 7.80 (d, J=8.0 Hz, 1H), 6.88 (d, J=8.0 Hz, 1H), 6.74 (s, 1H), 2.36 (s, 3H), 1.69 (s, 7H).

### Step 2: 7-(bromomethyl)-2,2-dimethyl-4H-benzo[d][1,3]dioxin-4-one

To a solution of 2,2,7-trimethyl-4H-benzo[d][1,3]dioxin-4-one (200 mg, 1.0 mmol) in CCl₄ (2 mL) was added NBS (221 mg, 1.2 mmol) and AIBN (34 mg, 0.2 mmol). The reaction was heated at 75° C. for 4 h then was diluted with water (30 mL) and extracted with DCM (50 mL×3). The combined organic layers were washed with brine, dried over Na₂SO₄, filtered and concentrated. The mixture was purified by prep-TLC (eluent: Pet. ether/EtOAc=15:1) to afford 7-(bromomethyl)-2,2-dimethyl-4H-benzo[d][1,3]dioxin-4-one (163 mg, 58%) as yellow solid. ¹H NMR (400 MHz, CDCl₃) δ 7.92 (d, J=8.0 Hz, 1H), 7.14 (d, J=8.0 Hz, 1H), 7.00 (s, 1H), 4.43 (s, 2H), 1.75-1.71 (m, 9H).

### Step 3: benzyl 1H-pyrazole-4-carboxylate

To a solution of 1H-pyrazole-4-carboxylic acid (1 g, 8.9 mmol) in DMSO (10 mL) was added KHCO₃ (1.1 g, 10.7 mmol) and the mixture was stirred for 30 min. (bromomethyl)benzene (1.4 g, 8.5 mmol) was added and stirring continued overnight. The reaction was diluted with water (30 mL) and extracted with EtOAc (50 mL×3). The combined organic layers were washed with brine, dried over Na₂SO₄, filtered and concentrated to afford benzyl 1H-pyrazole-4-carboxylate (1.2 g, 67%) as yellow oil. LCMS: m/z=202.8 [M+H]⁺; ¹H NMR (400 MHz, CDCl₃) δ 8.09 (s, 2H), 7.43-7.32 (m, 5H), 5.31 (s, 2H).

### Step 4: benzyl 1-((2,2-dimethyl-4-oxo-4H-benzo[d][1,3]dioxin-7-yl)methyl)-1H-pyrazole-4-carboxylate

To a solution of benzyl 1H-pyrazole-4-carboxylate (80 mg, 0.4 mmol) in MeCN (2 mL) was added 7-(bromomethyl)-2,2-dimethyl-4H-benzo[d][1,3]dioxin-4-one (141 mg, 0.5 mmol) and K₂CO₃ (111 mg, 0.8 mmol) and the reaction stirred at room temperature overnight. The solvent was removed and the residue obtained was purified by prep-TLC (eluent: Pet. Ether/EtOAc=2:1) to afford benzyl 1-((2,2-dimethyl-4-oxo-4H-benzo[d][1,3]dioxin-7-yl)methyl)-1H-pyrazole-4-carboxylate (99 mg, 64%) as colorless oil. ¹H NMR (400 MHz, CDCl₃) δ 7.98 (d, J=14.0 Hz, 2H), 7.94 (d, J=8.0 Hz, 1H), 7.44-7.31 (m, 5H), 6.94 (d, J=8.0 Hz, 1H), 6.71 (s, 1H), 5.33 (s, 2H), 5.28 (s, 2H), 1.71 (s, 6H).

### Step 5: 1-((2,2-dimethyl-4-oxo-4H-benzo[d][1,3]dioxin-7-yl)methyl)-1H-pyrazole-4-carboxylic acid

To a solution of benzyl 1-((2,2-dimethyl-4-oxo-4H-benzo[d][1,3]dioxin-7-yl)methyl)-1H-pyrazole-4-carboxylate (50 mg, 0.1 mmol) in EtOAc (1 mL) was added 10% Pd/C (5 mg). The mixture was stirred under a H₂ atmosphere at room temperature for 5 h then the catalyst was removed by filtration through celite and the filtrate concentrate to afford 1-((2,2-dimethyl-4-oxo-4H-benzo[d][1,3]dioxin-7-yl)methyl)-1H-pyrazole-4-carboxylic acid (30 mg, 77%) as colorless oil. ¹H NMR (400 MHz, CDCl₃) δ 8.02 (d, J=8.7 Hz, 2H), 7.96 (d, J=8.0 Hz, 1H), 6.96 (d, J=8.2 Hz, 1H), 6.74 (s, 1H), 5.36 (s, 2H), 1.72 (s, 6H).

### Synthesis of 1-(tetrahydro-2H-pyran-2-yl)-1H-pyrazole-4-carboxylic acid

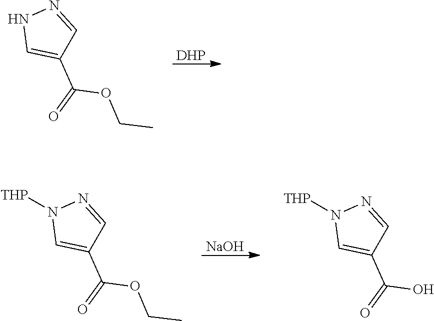

### Step 1: ethyl 1-(tetrahydro-2H-pyran-2-yl)-1H-pyrazole-4-carboxylate

To a solution of ethyl 1H-pyrazole-4-carboxylate (2.0 g, 23.78 mmol) in dry THE was added 3,4-dihydro-2H-pyran (5.0 g, 35.66 mmol) and TFA (2.8 g, 24.28 mmol) and the reaction heated at 80° C. overnight. The mixture was diluted with water and extracted with DCM. The combined organic layers were washed with brine, dried over Na₂SO₄, filtered and concentrated. The residue was purified by RP-column to afford ethyl 1-(tetrahydro-2H-pyran-2-yl)-1H-pyrazole-4-carboxylate (1.9 g, quant.) as a yellow oil. LCMS m/z=225.2 [M+H]⁺; ¹H NMR (400 MHz, CDCl₃) δ 8.10 (s, 1H), 7.92 (s, 1H), 5.41-5.33 (m, 1H), 4.28 (q, J=7.2 Hz, 2H), 3.74-3.64 (m, 1H), 2.12-1.97 (m, 4H), 1.74-1.58 (m, 3H), 1.32 (t, J=7.1 Hz, 3H).

### Step 2: 1-(tetrahydro-2H-pyran-2-yl)-1H-pyrazole-4-carboxylic acid

To a solution of ethyl 1-(tetrahydro-2H-pyran-2-yl)-1H-pyrazole-4-carboxylate (1.9 g, 8.47 mmol) in a mixture of THF, water and EtOH (12 mL/3 mL/3 mL) was added NaOH (6.8 g, 16.94 mmol). The reaction mixture was stirred at room temperature for 2 h then was diluted with water (50 mL) and extracted with ether (30 mL). The aqueous layer was collected and acidified to pH˜2 with 1M HCl then extracted with EtOAc (100 mL×3). The combined organic layers was washed with brine, dried over Na₂SO₄, filtered and concentrated to afford 1-(tetrahydro-2H-pyran-2-yl)-1H-pyrazole-4-carboxylic acid (940 mg, 40%) as a white solid. LCMS m/z=195.1 [M−H]⁻; ¹H NMR (400 MHz, CDCl₃) δ 8.19 (s, 1H), 8.00 (s, 1H), 5.45-5.39 (m, 1H), 4.12-4.02 (m, 1H), 3.78-3.64 (m, 1H), 2.17-1.98 (m, 3H), 1.76-1.59 (m, 3H).

### Synthesis of 2-(3-(tetrahydro-2H-pyran-4-yl)-1H-pyrazol-1-yl)propanoic acid

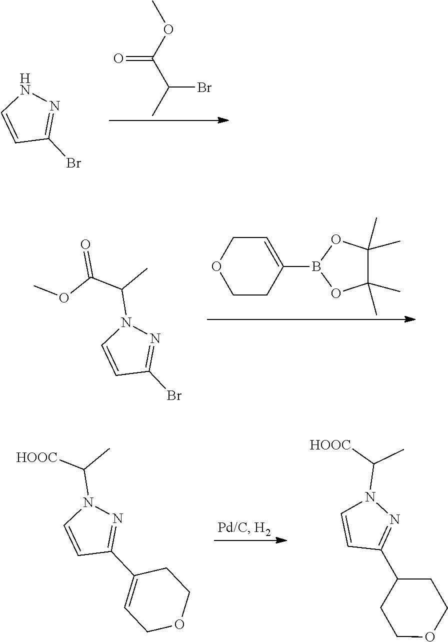

### Step 1: methyl 2-(3-bromo-1H-pyrazol-1-yl)propanoate

To a solution of 3-bromo-1H-pyrazole (5.0 g, 34.02 mmol) in DMF (50 mL) was added K₂CO₃ (9.4 g, 68.04 mmol) and methyl 2-bromopropanoate (6.25 g, 37.42 mmol). The reaction was stirred at room temperature for 4 h then was diluted with water (250 mL) and extracted with EtOAc (300 mL×3). The combined organic layers were washed with brine, dried over Na₂SO₄, filtered and concentrated. The residue was purified by column chromatography on silica gel (eluent: Pet. Ether:EtOAc=3:1, v/v) to afford methyl 2-(3-bromo-1H-pyrazol-1-yl)propanoate (7.0 g, 88%) as yellow oil. LCMS m/z=233.0 [M+H]⁺; ¹H NMR (400 MHz, Chloroform-d) δ 7.44 (d, J=2.4 Hz, 1H), 6.33 (d, J=2.4 Hz, 1H), 5.06 (q, J=7.4 Hz, 1H), 3.79-3.67 (m, 3H), 1.78 (d, J=7.4 Hz, 3H).

### Step 2: 2-(3-(3,6-dihydro-2H-pyran-4-yl)-1H-pyrazol-1-yl)propanoic acid

To a solution of methyl 2-(3-bromo-1H-pyrazol-1-yl)propanoate (1.0 g, 4.29 mmol), K₂CO₃ (1.19 g, 8.58 mmol) and Pd(dppf)Cl₂ (312.0 mg, 0.429 mmol) in a mixture of 1,4-dioxane (10 mL) and water (2 mL) was added 2-(3,6-dihydro-2H-pyran-4-yl)-4,4,5,5-tetramethyl-1,3,2-dioxaborolane (1.08 g, 5.15 mmol). The reaction was heated at 100° C. under N₂ atmosphere overnight. 10% aq. NaOH (5 mL) was added and the reaction stirred at room temperature for another 4 h. The mixture was diluted with water (50 mL) and extracted with Et₂O (50 mL×2). The aqueous layer was collected and acidified with 1M HCl to pH˜2 then extracted with EtOAc (100 mL×2). The combined organic layers were washed with brine, dried over Na₂SO₄, filtered and concentrated. The residue was purified by RP column to afford 2-(3-(3,6-dihydro-2H-pyran-4-yl)-1H-pyrazol-1-yl)propanoic acid (800 mg, 84%) as a yellow oil. LCMS m/z=223.1 [M+H]⁺; ¹H NMR (400 MHz, Chloroform-d) δ 7.47 (d, J=2.4 Hz, 1H), 6.39 (d, J=2.4 Hz, 1H), 6.33-6.25 (m, 1H), 5.04 (d, J=7.2 Hz, 1H), 4.31 (q, J=2.6 Hz, 2H), 3.91 (t, J=5.4 Hz, 2H), 2.55 (s, 2H), 1.81 (d, J=7.2 Hz, 3H).

### Step 3: 2-(3-(tetrahydro-2H-pyran-4-yl)-1H-pyrazol-1-yl)propanoic acid

To a solution of 2-(3-(3,6-dihydro-2H-pyran-4-yl)-1H-pyrazol-1-yl)propanoic acid (500 mg, 2.25 mmol) in MeOH (5 mL) was added 10% Pd/C (60 mg) and the reaction stirred under a H₂ atmosphere for 4 h. The catalyst was removed by filtration through celite and the filtrate concentrated to afford 2-(3-(tetrahydro-2H-pyran-4-yl)-1H-pyrazol-1-yl)propanoic acid (420 mg, 83%) as a yellow oil which was used directly in the next step. LCMS m/z=225.1 [M+H]⁺; ¹H NMR (400 MHz, Chloroform-d) δ 7.45 (d, J=2.4 Hz, 1H), 6.17 (d, J=2.4 Hz, 1H), 4.99 (q, J=7.2 Hz, 1H), 4.07-3.93 (m, 2H), 3.58-3.45 (m, 2H), 3.03-2.84 (m, 1H), 1.92-1.83 (m, 2H), 1.84-1.70 (m, 5H).

### Synthesis of 1-(azidomethyl)-4-(trifluoromethyl)benzene

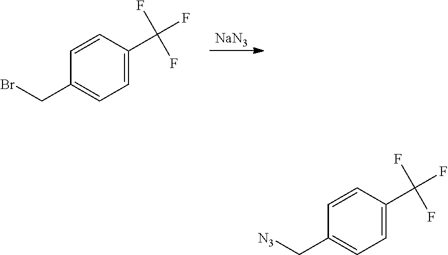 [SMILES: FC(F)(F)c1ccc(CBr)cc1 ; [N-]=[N+]=NCc1ccc(C(F)(F)F)cc1 ; "NaN3"]

### Step 1: 1-(azidomethyl)-4-(trifluoromethyl)benzene

To a solution of 1-(bromomethyl)-4-(trifluoromethyl)benzene (200 mg, 0.836 mmol) in DMF (3 mL) was added sodium azide (65.27 mg, 1.0 mmol) and the reaction heated at 80° C. overnight. The mixture was diluted with water (30 mL) and extracted with EtOAc (50 mL×2). The combined organic layers were washed with brine, dried over Na₂SO₄ and concentrated to afford 1-(azidomethyl)-4-(trifluoromethyl)benzene (114 mg, 68%) as a white oil. 1H NMR (400 MHz, DMSO-d6) δ 7.78 (d, J=8.0 Hz, 2H), 7.60 (d, J=7.9 Hz, 2H), 4.60 (s, 2H).

Table 34: The building blocks listed in Table 34 were synthesized from corresponding halides according to the procedures outlined for 1-(azidomethyl)-4-(trifluoromethyl)benzene using the appropriate commercially available reagents and/or intermediates described elsewhere.

TABLE 34

| Example | | | | |
|---|---|---|---|---|
| Number | Compound | Client No. | ¹HNMR | LCMS |
| | 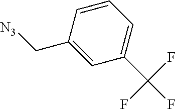 [SMILES: [N-]=[N+]=NCc1cccc(C(F)(F)F)c1] | ZJ-1583-035 | ¹H NMR (400 MHz, Chloroform-d) δ 7.63 − 7.57 (m, 2H), 7.51 (d, J = 4.4 Hz, 2H), 4.43 (s, 2H). | |
| | 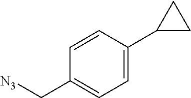 [SMILES: [N-]=[N+]=NCc1ccc(C2CC2)cc1] | SJR-1687-107 | ¹H NMR (400 MHz, DMSO-d₆) δ 7.26 − 7.22 (m, 2H), 7.12 − 7.08 (m, 2H), 4.36 (s, 2H), 1.96 − 1.87 (m, 1H), 0.97 − 0.91 (m, 2H), 0.69 − 0.64 (m, | |
| | | | 2H). | |
| | 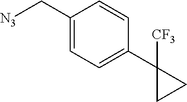 [SMILES: [N-]=[N+]=NCc1ccc(C2(C(F)(F)F)CC2)cc1] | LL-1544-129 | ¹H NMR (400 MHz, DMSO-d₆) δ 7.51 − 7.47 (m, 1H), 7.41 − 7.36 (m, 1H), 4.46 (s, 1H), 1.36 − 1.31 (m, 1H), 1.14 − 1.10 (m, 1H). | |

### Synthesis of 1-(bromomethyl)-4-cyclopropylbenzene

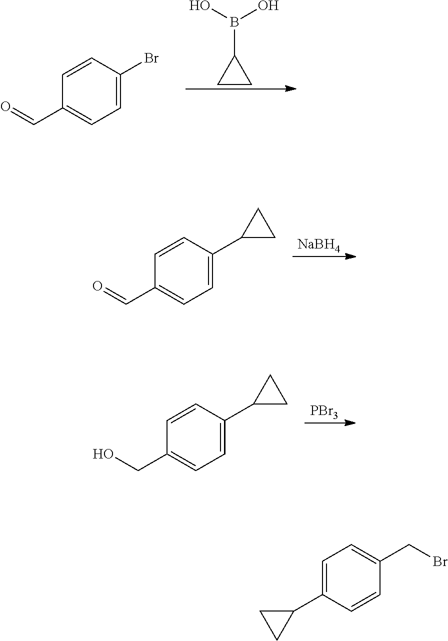

### Step 1: 4-cyclopropylbenzaldehyde

To a solution of 4-bromobenzaldehyde (1.0 g, 5.4 mmol) in a mixture of toluene and water (9 mL/3 mL) was added cyclopropylboronic acid (1.2 g, 8.11 mmol), tricyclohexyl phosphine (61 mg, 0.2 mmol) and K₂CO₃ (1.5 g, 10.81 mmol) and Pd(OAc)₂ (25 mg, 0.11 mmol). The reaction was heated at 90° C. overnight then cooled to room temperature, diluted with water (50 mL) and extracted with EtOAc (80 mL×2). The combined organic layers were washed with brine, dried over Na₂SO₄, filtered and concentrated. The mixture was purified by column chromatography on silica gel (eluent: Pet. Ether:EtOAc=100:1, v/v) to afford 4-cyclopropylbenzaldehyde (736 mg, 93%) as a white solid. LCMS m/z=147.1 [M+H]⁺.

### Step 2: (4-cyclopropylphenyl)methanol

To a solution of 4-cyclopropylbenzaldehyde (500 mg, 3.42 mmol) in a mixture of MeOH and THF (2.5 mL/2.5 mL) was added NaBH₄ (130 mg, 3.42 mmol). The reaction was stirred at room temperature for 30 min then was diluted with water (50 mL) and extracted with EtOAc (100 mL×2). The combined organic layers were washed with brine, dried over Na₂SO₄, filtered and concentrated. The residue was purified by column chromatography on silica gel (eluent: DCM/MeOH=50/1, v/v) to afford (4-cyclopropylphenyl)methanol (422 mg, 83%) as a yellow oil. ¹H NMR (400 MHz, DMSO-d₆) δ 7.19-7.15 (m, 2H), 7.03-6.99 (m, 2H), 5.05 (t, J=5.7 Hz, 1H), 4.42 (d, J=5.7 Hz, 2H), 1.88 (tt, J=8.4, 5.1 Hz, 1H), 0.94-0.88 (m, 2H), 0.65-0.60 (m, 2H).

### Step 3: 1-(bromomethyl)-4-cyclopropylbenzene

To a solution of (4-cyclopropylphenyl)methanol (20 mg, 0.13 mmol) in Et₂O (0.5 mL) at 0° C. was added PBr₃ (37 mg, 0.13 mmol). The reaction was allowed to warm to room temperature and was stirred for 2 h then diluted with water (20 mL) and extracted with EtOAc (30 mL×2). The combined organic layers were washed with brine, dried over Na₂SO₄, filtered and concentrated to afford 1-(bromomethyl)-4-cyclopropylbenzene (19.8 mg, 70%) as a yellow oil. ¹H NMR (400 MHz, DMSO-d₆) δ 7.33-7.29 (m, 2H), 7.08-7.03 (m, 2H), 4.67 (s, 2H), 1.95-1.86 (m, 1H), 0.98-0.92 (m, 2H), 0.69-0.64 (m, 2H).

### Synthesis of 1-(bromomethyl)-4-(1-(trifluoromethyl)cyclopropyl)benzene

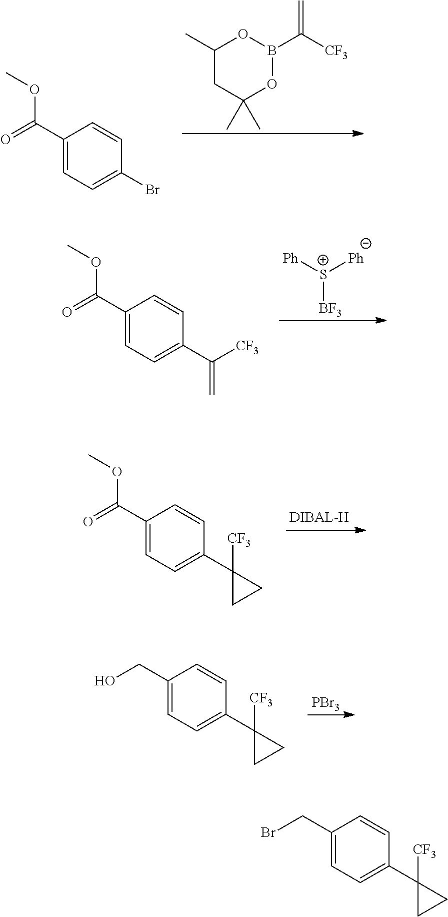

### Step 1: methyl 4-(3,3,3-trifluoroprop-1-en-2-yl)benzoate

To a solution of methyl 4-bromobenzoate (1.00 g, 4.65 mmol) in a mixture of 1,4-dioxane (8 mL) and water (4 mL) was added Pd(PPh₃)₄ (538 mg, 0.46 mmol), 4,4,6-trimethyl-2-(3,3,3-trifluoroprop-1-en-2-yl)-1,3,2-dioxaborinane (1.34 g, 6.05 mmol) and Na₂CO₃ (986 mg, 9.30 mmol) and the reaction heated at 110° C. in a microwave reactor for 1 hour. The mixture was diluted with water (50 mL) and extracted with EtOAc (80 mL×3). The combined organic layers were washed with brine, dried over Na₂SO₄, filtered and concentrated. The residue was purified by column chromatography on silica gel (eluent: Pet. Ether/EtOAc=25/1, v/v) to afford methyl 4-(3,3,3-trifluoroprop-1-en-2-yl)benzoate (0.90 g, 84%) as a yellow oil. ¹H NMR (400 MHz, DMSO-d₆) δ 8.06-7.99 (m, 2H), 7.66 (d, J=8.0 Hz, 2H), 6.28-6.19 (m, 2H), 3.87 (s, 3H).

### Step 2: methyl 4-(1-(trifluoromethyl)cyclopropyl)benzoate

To a solution of methyl 4-(3,3,3-trifluoroprop-1-en-2-yl)benzoate (800 mg, 3.48 mmol) and diphenyl(methyl)sulfonium tetrafluoroborate in (1.30 g, 4.52 mmol) in fresh distilled THF (8 mL) at 0° C. under N₂ atmosphere was added NaHMDS in THF (2.0 M, 2.8 mL). The mixture was stirred at room temperature for 2 h then was diluted with saturated aq. NH₄Cl (30 mL) and extracted with EtOAc (50 mL×3). The combined organic layers were washed with brine, dried over Na₂SO₄, filtered and concentrated. The residue was purified by column chromatography on silica gel (eluent: Pet. Ether/EtOAc=50/1, v/v) to afford methyl 4-(1-(trifluoromethyl)cyclopropyl)benzoate (100 mg, 11%) as a yellow oil. ¹H NMR (400 MHz, Chloroform-d) δ 8.01 (d, J=8.0 Hz, 2H), 7.53 (d, J=8.0 Hz, 2H), 3.92 (s, 3H), 1.41-1.38 (m, 2H), 1.09-1.02 (m, 2H).

### Step 3: (4-(1-(trifluoromethyl)cyclopropyl)phenyl)methanol

To a solution of methyl 4-(1-(trifluoromethyl)cyclopropyl)benzoate (500 mg, 2.05 mmol) in fresh distilled THF (8 mL) at 0° C. under N₂ atmosphere was added a solution of DIBAL-H in THF (1.0 M, 4.1 mL, 4.1 mmol). The mixture was stirred at room temperature overnight then diluted with saturated aq. NH₄Cl (30 mL) and extracted with EtOAc (50 mL×3). The combined organic layers were washed with brine, dried over Na₂SO₄, filtered and concentrated. The residue was purified by column chromatography on silica gel (eluent: Pet. Ether/EtOAc=20/1, v/v) to afford (4-(1-(trifluoromethyl)cyclopropyl)phenyl)methanol (350 mg, 79%) as a yellow oil. ¹H NMR (400 MHz, DMSO-d₆) δ 7.44-7.37 (m, 2H), 7.35-7.29 (m, 2H), 5.19 (t, J=5.8 Hz, 1H), 4.49 (d, J=5.8 Hz, 2H), 1.34-1.29 (m, 2H), 1.11-1.05 (m, 2H).

### Step 4: 1-(bromomethyl)-4-(1-(trifluoromethyl)cyclopropyl)benzene

To a solution of (4-(1-(trifluoromethyl)cyclopropyl)phenyl)methanol (10 mg, 0.04 mmol) in Et₂O (0.5 mL) at 0° C. was added PBr₃ (13 mg, 0.04 mmol). The mixture was stirred at room temperature for 2 h then the solvent removed to afford 1-(bromomethyl)-4-(1-(trifluoromethyl)cyclopropyl)benzene (10 mg, 83%) as a colorless oil. ¹H NMR (400 MHz, DMSO-d₆) δ 7.45 (s, 4H), 4.70 (s, 2H), 1.35-1.32 (m, 2H), 1.12 (s, 2H).

### Synthesis of 3-((tert-butyldimethylsilyl)oxy)-1H-pyrazole-5-carboxylic acid

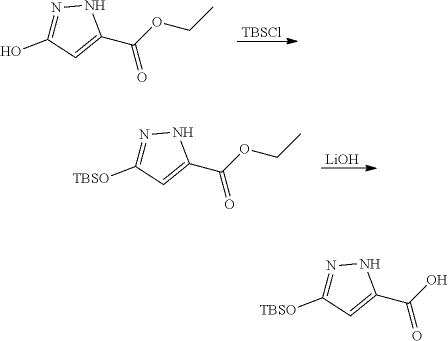

### Step 1: ethyl 3-((tert-butyldimethylsilyl)oxy)-1H-pyrazole-5-carboxylate

To a solution of ethyl 3-hydroxy-1H-pyrazole-5-carboxylate (500 mg, 3.2 mmol) in dry DMF (5 mL) at 0° C. was added 1H-imidazole (327 mg, 4.8 mmol) and TBS-Cl (579 mg, 3.8 mmol). The mixture was stirred at room temperature overnight then was diluted with water (30 mL) and extracted with EtOAc (50 mL×3). The combined organic layers were washed with brine, dried over Na₂SO₄, filtered and concentrated to afford ethyl 3-((tert-butyldimethylsilyl)oxy)-1H-pyrazole-5-carboxylate (770 mg, 89%) as a colourless oil. LCMS m/z=271.1 [M+H]⁺; ¹H NMR (400 MHz, DMSO-d₆) δ 13.04 (s, 1H), 4.27 (q, J=7.0 Hz, 2H), 1.28 (t, J=7.0 Hz, 3H), 0.93 (s, 9H), 0.21 (s, 6H).

### Step 2: 3-((tert-butyldimethylsilyl)oxy)-1H-pyrazole-5-carboxylic acid

To a solution of methyl ethyl 3-((tert-butyldimethylsilyl)oxy)-1H-pyrazole-5-carboxylate (150 mg, 0.55 mmol) in a mixture of THF (1 mL), EtOH (0.5 mL) and water (0.25 mL) was added lithium hydroxide monohydrate (70 mg, 1.66 mmol). The reaction was stirred at room temperature for 1 h then was concentrated to afford 3-((tert-butyldimethylsilyl)oxy)-1H-pyrazole-5-carboxylic acid (134 mg, 100%) which was used directly in the next step. LCMS m/z=241.1 [M−H]⁻.

### Synthesis of 6-((4-methoxybenzyl)oxy)pyrazine-2-carboxylic acid

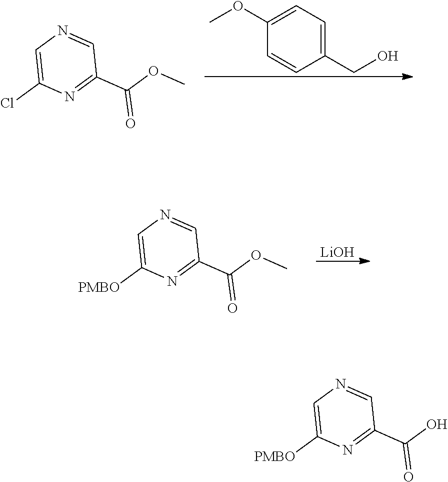

### Step 1: methyl 6-((4-methoxybenzyl)oxy)pyrazine-2-carboxylate

To a solution methyl 6-chloropyrazine-2-carboxylate (500 mg, 2.89 mmol) and t-BuXphos (246 mg, 0.58 mmol) in toluene (5 mL) was added (4-methoxyphenyl)methanol (480 mg, 3.48 mmol) and Pd(OAc)₂ (65 mg, 0.29 mmol). The reaction was heated at 100° C. for 4 h then was cooled to room temperature, diluted with water (50 mL) and extracted with EtOAc (80 mL×3). The combined organic layers were washed with brine, dried over Na₂SO₄, filtered and concentrated. The residue obtained was purified by column chromatography on silica gel (eluent: Pet. Ether:EtOAc=20:1 to 10:1) to afford methyl 6-((4-methoxybenzyl)oxy)pyrazine-2-carboxylate (226 mg, 29%) as a yellow oil. LCMS m/z=275 [M+H]⁺; ¹H NMR (400 MHz, DMSO-d6) δ 8.79 (d, J=5.6 Hz, 1H), 8.58-8.55 (m, 1H), 7.01-6.83 (m, 4H), 5.35 (s, 1H), 3.93 (s, 2H), 3.77-3.73 (m, 5H).

### Step 2: 6-((4-methoxybenzyl)oxy)pyrazine-2-carboxylic acid

To a solution of methyl 6-((4-methoxybenzyl)oxy)pyrazine-2-carboxylate (200 mg, 0.05 mmol) in a mixture of THE (2 mL), MeOH (0.5 mL) and H₂O (0.5 mL) was added LiOH (44 mg, 1.82 mmol). The reaction mixture was stirred at room temperature for 1.5 h then diluted with water (30 mL) and extracted with EtOAc (50 mL). The aqueous layer was collected and acidified with 2 M HCl to pH˜2 then extracted with EtOAc (60 mL×3). The combined organic layers were washed with brine, dried over Na₂SO₄, filtered and concentrated to afford 6-((4-methoxybenzyl)oxy)pyrazine-2-carboxylic acid (127 mg, 68%) as a white solid. LCMS m/z=261 [M+H]⁺; ¹H NMR (400 MHz, DMSO-d6) δ 8.76 (s, 1H), 8.52 (s, 1H), 7.47 (d, J=8.6 Hz, 2H), 6.95 (d, J=8.6 Hz, 2H), 5.36 (s, 2H), 3.76 (s, 3H).

### Synthesis of 4-methoxy-1-((2-(trimethylsilyl)ethoxy)methyl)-1H-pyrazole-5-carboxylic acid

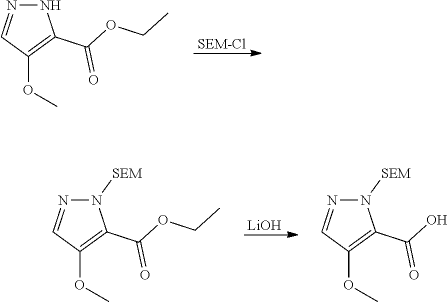

### Step 1: ethyl 4-methoxy-1-((2-(trimethylsilyl)ethoxy)methyl)-1H-pyrazole-5-carboxylate

To a solution of ethyl 4-methoxy-1H-pyrazole-5-carboxylate (50 mg, 0.29 mmol) in THE (2 mL) at 0° C. was added NaH (15 mg, 0.32 mmol) and the mixture stirred for 30 min. (2-(chloromethoxy)ethyl)trimethylsilane (0.06 mL, 0.35 mmol) was added and the reaction allowed to warm to room temperature then stirred for 1 h. The reaction was diluted with water (20 mL) and extracted with EtOAc (30 mL×2). The combined organic layers were washed with brine, dried over Na₂SO₄ filtered and concentrated to afford crude ethyl 4-methoxy-1-((2-(trimethylsilyl)ethoxy)methyl)-1H-pyrazole-5-carboxylate (100 mg, 100%) as a colorless oil. LCMS m/z=301.1 [M+H]⁺; ¹H NMR (400 MHz, DMSO-d₆) δ 8.31 (s, 1H), 5.26 (s, 2H), 4.16 (q, J=7.2 Hz, 2H), 3.85 (s, 3H), 3.55 (dd, J=10.2, 5.8 Hz, 2H), 1.23 (t, J=7.2 Hz, 3H), 0.84 (t, J=8.0 Hz, 2H), −0.04 (d, J=4.0 Hz, 9H).

### Step 2: 4-methoxy-1-((2-(trimethylsilyl)ethoxy)methyl)-1H-pyrazole-5-carboxylic acid

To a solution of ethyl 4-methoxy-1-((2-(trimethylsilyl)ethoxy)methyl)-1H-pyrazole-5-carboxylate (100 mg, 0.33 mmol) in a mixture of THF (4 mL), EtOH (1 mL) and H₂O (1 mL) was added LiOH (28 mg, 0.67 mmol) and the reaction heated at 40° C. for 4 h. The reaction was diluted with water (30 mL) and extracted with EtOAc (50 mL). The aqueous phase was acidified to pH˜2 with 1M HCl and extracted with EtOAc (50 mL×3). The combined organic layers were washed with brine, dried over Na₂SO₄ filtered and concentrated to afford 4-methoxy-1-((2-(trimethylsilyl)ethoxy)methyl)-1H-pyrazole-5-carboxylic acid (75 mg, 83%) as a white solid. LCMS m/z=273.1 [M+H]⁺; ¹H NMR (400 MHz, DMSO-d₆) δ 8.09 (s, 1H), 5.22 (s, 2H), 3.81 (s, 3H), 3.53 (t, J=8.0 Hz, 2H), 0.84 (d, J=8.0 Hz, 2H), −0.04 (d, J=4.0 Hz, 9H).

### Synthesis of 2-(3-cyclopentyl-1H-pyrazol-1-yl)acetic acid

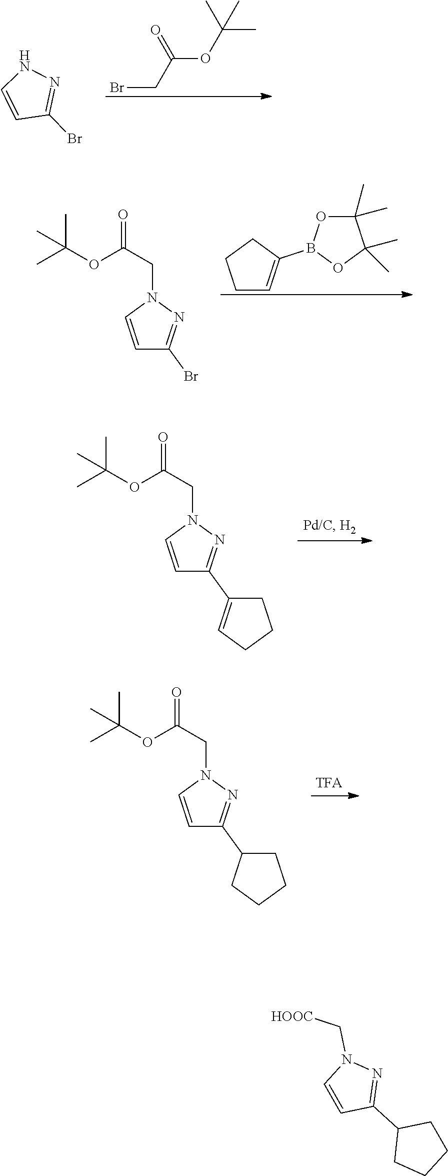

### Step 1: tert-butyl 2-(3-bromo-1H-pyrazol-1-yl)acetate

To a solution of 3-bromo-1H-pyrazole (5.0 g, 34.02 mmol) and tert-butyl 2-bromoacetate (8.0 g, 40.82 mmol) in DMF (40 mL) was added K₂CO₃ (9.4 g, 68.04 mmol). The reaction was stirred at room temperature overnight then diluted with water (100 mL) and extracted with EtOAc (200 mL×2). The combined organic layers were washed with brine, dried over Na₂SO₄ filtered and concentrated. The residue was purified by RP-column to afford tert-butyl 2-(3-bromo-1H-pyrazol-1-yl)acetate (7.4 g, 84%) as a white solid. ¹H NMR (400 MHz, DMSO-d₆) δ 7.74 (d, J=2.4 Hz, 1H), 6.40 (d, J=2.4 Hz, 1H), 4.94 (s, 2H), 1.42 (s, 9H).

### Step 2: tert-butyl 2-(3-(cyclopent-1-en-1-yl)-1H-pyrazol-1-yl)acetate

To a solution of tert-butyl 2-(3-bromo-1H-pyrazol-1-yl)acetate (500 mg, 1.92 mmol), 2-(cyclopent-1-en-1-yl)-4,4,5,5-tetramethyl-1,3,2-dioxaborolane (560 mg, 2.88 mmol) and K₂CO₃ (797 mg, 5.77 mmol) in a mixture of dioxane and water (2.5 mL/0.5 mL) was added Pd(dppf)Cl₂ (139.5 mg, 0.19 mmol). The reaction heated at 110° C. overnight then was diluted with water (30 mL) and extracted with EtOAc (50 mL×3). The combined organic layers were washed with brine, dried over Na₂SO₄ filtered and concentrated. The residue was purified by column chromatography on silica gel (eluent: Pet.Ether:EtOAc=25:1) to afford tert-butyl 2-(3-(cyclopent-1-en-1-yl)-1H-pyrazol-1-yl)acetate (320 mg, 67%) as a white solid. ¹H NMR (400 MHz, DMSO-d₆) δ 7.63 (d, J=2.2 Hz, 1H), 6.40 (d, J=2.2 Hz, 1H), 6.09 (p, J=2.2 Hz, 1H), 4.89 (s, 2H), 2.60 (td, J=7.6, 2.4 Hz, 2H), 2.43 (tq, J=7.6, 2.6 Hz, 2H), 1.90 (p, J=7.4 Hz, 2H), 1.42 (s, 9H).

### Step 3: tert-butyl 2-(3-cyclopentyl-1H-pyrazol-1-yl)acetate

To a solution of tert-butyl 2-(3-(cyclopent-1-en-1-yl)-1H-pyrazol-1-yl)acetate (1.5 g, 6.04 mmol) in MeOH (12 mL) was added 10% Pd/C (600 mg). The reaction was stirred under a H₂ atmosphere for 2 h then the catalyst was removed by filtration through celite and the filtrate concentrated to afford tert-butyl 2-(3-cyclopentyl-1H-pyrazol-1-yl)acetate (1.24 g, 82%) which was used without further purification. ¹H NMR (400 MHz, DMSO-d₆) δ 7.55 (d, J=2.2 Hz, 1H), 6.06 (d, J=2.2 Hz, 1H), 4.81 (s, 2H), 1.92 (tq, J=9.0, 4.6 Hz, 2H), 1.71-1.54 (m, 6H), 1.41 (s, 9H).

### Step 4: 2-(3-cyclopentyl-1H-pyrazol-1-yl)acetic acid

To a solution of tert-butyl 2-(3-cyclopentyl-1H-pyrazol-1-yl)acetate (1.5 g, 6.00 mmol) in DCM (8 mL) was added TFA (4 mL) and the reaction stirred at room temperature for 6 h. The solvent was removed under vacuum to afford 2-(3-cyclopentyl-1H-pyrazol-1-yl)acetic acid (1.2 g, 100%) as a brown oil, which was used in the next step without further purification.

### Synthesis of 1-(4-(tert-butoxycarbonyl)benzyl)-1H-pyrazole-4-carboxylic acid

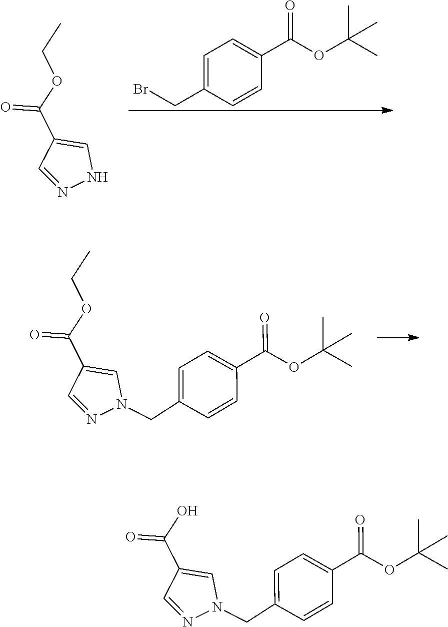

### Step 1: ethyl 1-(4-(tert-butoxycarbonyl)benzyl)-1H-pyrazole-4-carboxylate

To a solution of ethyl 1H-pyrazole-4-carboxylate (1.0 g, 7.14 mmol) and tert-butyl 4-(bromomethyl)benzoate (1.9 g, 7.14 mmol) in DMF (15 mL) was added potassium carbonate (2.9 g, 21.41 mmol). The reaction mixture was stirred at room temperature overnight. The mixture was diluted with water (100 mL) and extracted with EtOAc (200 mL×2). The combined organic layers were dried over Na₂SO₄, filtered and concentrated. The residue was purified by RP-column (40% MeCN in water) to afford ethyl 1-(4-(tert-butoxycarbonyl)benzyl)-1H-pyrazole-4-carboxylate (2.1 g, 89%) as a white solid. LCMS m/z=331.2 [M+H]⁺; ¹H NMR (400 MHz, DMSO-d6) δ 8.49 (s, 1H), 7.89-7.84 (m, 3H), 7.35 (d, J=8.0 Hz, 2H), 5.45 (s, 2H), 4.21 (q, J=7.0 Hz, 2H), 1.53 (s, 9H), 1.26 (t, J=7.0 Hz, 3H).

### Step 2: 1-(4-(tert-butoxycarbonyl)benzyl)-1H-pyrazole-4-carboxylic acid

To a solution of ethyl 1-(4-(tert-butoxycarbonyl)benzyl)-1H-pyrazole-4-carboxylate (1.5 g, 4.54 mmol) in a mixture of THF (4 mL), EtOH (1 mL) and H₂O (1 mL) was added LiOH (953 mg, 0.67 mmol). The reaction was stirred at room temperature for 2 h then was diluted with water (70 mL) and extracted with EtOAc (70 mL). The aqueous phase was acidified to pH˜2 with 1M HCl and extracted with EtOAc (100 mL×3). The combined organic layers were washed with brine, dried over Na₂SO₄ and concentrated to afford 1-(4-(tert-butoxycarbonyl)benzyl)-1H-pyrazole-4-carboxylic acid (1300 mg, 95%) as a white solid. LCMS m/z=391.15 [M+H]⁺; ¹H NMR (400 MHz, DMSO-d6) δ 12.30 (s, 1H), 8.40 (s, 1H), 7.89-7.82 (m, 3H), 7.34 (d, J=8.0 Hz, 2H), 5.44 (s, 2H), 1.53 (s, 9H).

### Synthesis of 1-(3-(methoxycarbonyl)benzyl)-1H-pyrazole-4-carboxylic acid

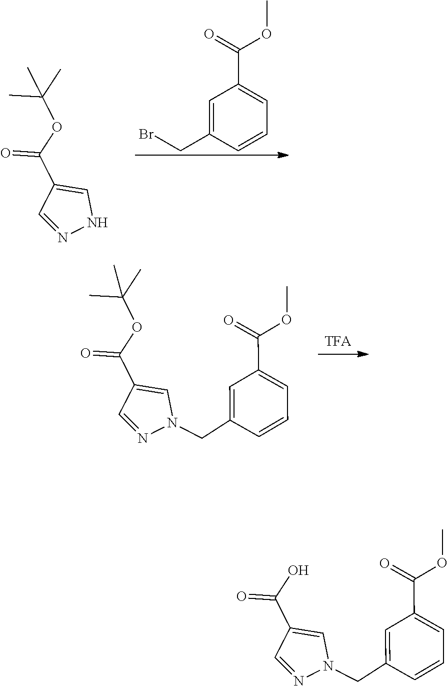 [SMILES: CC(C)(C)OC(=O)c1cn[nH]c1 ; COC(=O)c1cccc(CBr)c1 ; COC(=O)c1cccc(Cn2cc(C(=O)OC(C)(C)C)cn2)c1 ; COC(=O)c1cccc(Cn2cc(C(=O)O)cn2)c1]

### Step 1: tert-butyl 1-(3-(methoxycarbonyl)benzyl)-1H-pyrazole-4-carboxylate

To a solution of methyl 3-(bromomethyl)benzoate (200 mg, 0.88 mmol) and tert-butyl 1H-pyrazole-4-carboxylate (220 mg, 1.32 mmol) in DMF (5 mL) was added K₂CO₃ (364 mg, 2.64 mmol) and KI (2 mg, 0.01 mmol). The reaction mixture was stirred at room temperature for 2 h then was diluted with water (30 mL) and extracted with EtOAc (50 mL×2). The combined organic layers were washed with brine, dried over Na₂SO₄, filtered and concentrated. The residue was purified by column chromatography on silica gel (eluent: DCM:MeOH=50:1) to afford tert-butyl 1-(3-(methoxycarbonyl)benzyl)-1H-pyrazole-4-carboxylate (240 mg, 91%) as a yellow oil. LCMS m/z=317.1 [M+H]⁺; ¹H NMR (400 MHz, DMSO-d₆) δ 8.40 (s, 1H), 7.89 (dd, J=7.2, 1.6 Hz, 2H), 7.80 (s, 1H), 7.59-7.47 (m, 2H), 5.44 (s, 2H), 3.85 (s, 3H), 1.48 (s, 9H).

### Step 2: 1-(3-(methoxycarbonyl)benzyl)-1H-pyrazole-4-carboxylic acid

To a solution of tert-butyl 1-(3-(methoxycarbonyl)benzyl)-1H-pyrazole-4-carboxylate (30 mg, 0.095 mmol) in DCM (2 mL) was added TFA (0.5 mL) and the reaction stirred at room temperature for 2 h. The solvent was removed under vacuum to afford 1-(3-(methoxycarbonyl)benzyl)-1H-pyrazole-4-carboxylic acid (24 mg, 100%) which was used directly in the next step. LCMS m/z=261.10 [M+H]⁺.

### Synthesis of 1-(2-(methoxycarbonyl)benzyl)-1H-pyrazole-4-carboxylic acid

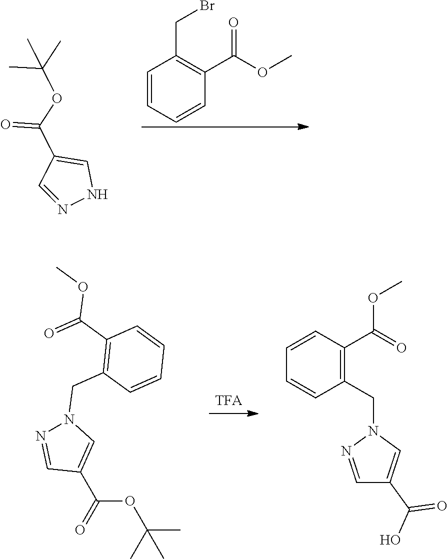 [SMILES: CC(C)(C)OC(=O)c1cn[nH]c1 ; COC(=O)c1ccccc1CBr ; COC(=O)c1ccccc1Cn1cc(C(=O)OC(C)(C)C)cn1 ; COC(=O)c1ccccc1Cn1cc(C(=O)O)cn1]

1-(2-(methoxycarbonyl)benzyl)-1H-pyrazole-4-carboxylic acid was synthesized from tert-butyl 1H-pyrazole-4-carboxylate according to the procedures outlined for 1-(3-(methoxycarbonyl)benzyl)-1H-pyrazole-4-carboxylic acid using the appropriate commercially available reagents and/or intermediates described elsewhere.

### Step 1: tert-butyl 1-(2-(methoxycarbonyl)benzyl)-1H-pyrazole-4-carboxylate

LCMS m/z=317.1 [M+H]⁺; ¹HNMR (400 MHz, Chloroform-d) δ 8.01 (dd, J=7.8, 1.4 Hz, 1H), 7.89 (d, J=14.4 Hz, 2H), 7.47 (td, J=7.6, 1.4 Hz, 1H), 7.37 (td, J=7.6, 1.3 Hz, 1H), 6.97 (d, J=7.8 Hz, 1H), 5.74 (s, 2H), 3.90 (s, 3H), 1.53 (s, 9H).

### Step 2: 1-(2-(methoxycarbonyl)benzyl)-1H-pyrazole-4-carboxylic acid

LCMS m/z=261.1 [M+H]⁺.

### Synthesis of 1-(4-((1-methoxy-2-methyl-1-oxopropan-2-yl)oxy)benzyl)-1H-pyrazole-4-carboxylic acid

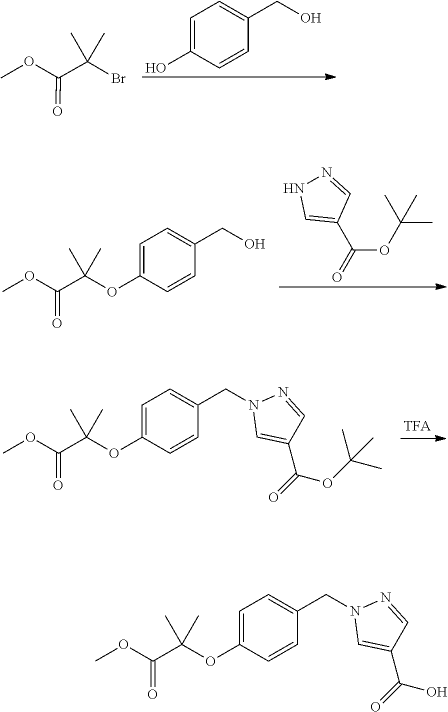

### Step 1: methyl 2-(4-(hydroxymethyl)phenoxy)-2-methylpropanoate

To a solution of 4-(hydroxymethyl)phenol (3.43 g, 27.60 mmol) in acetonitrile (50 mL) was added methyl 2-bromo-2-methylpropanoate (5.0 g, 27.60 mmol) and K₂CO₃ (11.45 g, 82.90 mmol). The reaction was heated at 80° C. overnight then was diluted with water (50 mL) and extracted with EtOAc (100 ml×2). The combined organic layers were washed with brine, dried over Na₂SO₄, filtered and concentrated. The residue obtained was purified by column chromatography on silica gel (eluent: Pet. Ether:EtOAc=8:1) to afford methyl 2-(4-(hydroxymethyl)phenoxy)-2-methylpropanoate (2.6 g, 42%) as colourless oil. ¹H NMR (400 MHz, DMSO-d6) δ 7.20 (d, J=8.2 Hz, 2H), 6.74 (d, J=8.6 Hz, 2H), 5.06 (s, 1H), 4.41 (d, J=5.6 Hz, 2H), 3.69 (s, 3H), 1.50 (s, 6H).

### Step 2: tert-butyl 1-(4-((1-methoxy-2-methyl-1-oxopropan-2-yl)oxy)benzyl)-1H-pyrazole-4-carboxylate

To a solution of methyl 2-(4-(hydroxymethyl)phenoxy)-2-methylpropanoate (500 mg, 2.23 mmol) and tert-butyl 1H-pyrazole-4-carboxylate (375 mg, 2.23 mmol) in dry THF (5.0 mL) at 0° C. under an atmosphere of N₂ was added PPh₃ (585 mg, 2.23 mmol) and DIAD (676 mg, 3.34 mmol). The reaction mixture was stirred at room temperature overnight then was diluted with water (30 mL) and extracted with EtOAc (50 mL×2). The combined organic layers were washed with brine, dried over Na₂SO₄, filtered and concentrated. The residue was purified by prep-TLC (eluent: Pet. Ether:EtOAc=2:1) to afford tert-butyl 1-(4-((1-methoxy-2-methyl-1-oxopropan-2-yl)oxy)benzyl)-1H-pyrazole-4-carboxylate (420 mg, 51%) as colourless oil. LCMS m/z=375.1 [M+H]⁺; ¹H NMR (400 MHz, DMSO-d6) δ58.30 (s, 1H), 7.76 (s, 1H), 7.18 (d, J=8.2 Hz, 2H), 6.75 (d, J=8.2 Hz, 2H), 5.26 (s, 2H), 3.68 (s, 3H), 1.49 (d, J=9.6 Hz, 15H).

### Step 3: 1-(4-((1-methoxy-2-methyl-1-oxopropan-2-yl)oxy)benzyl)-1H-pyrazole-4-carboxylic acid

To a solution of tert-butyl 1-(4-((1-methoxy-2-methyl-1-oxopropan-2-yl)oxy)benzyl)-1H-pyrazole-4-carboxylate (100 mg, 0.267 mmol) in DCM (2.0 mL) was added TFA (1.0 mL) and the reaction stirred at room temperature for 1 h. The solvent was removed under vacuum to afford 1-(4-((1-methoxy-2-methyl-1-oxopropan-2-yl)oxy)benzyl)-1H-pyrazole-4-carboxylic acid (85.0 mg, 100%) which was used directly in the next step. LCMS m/z=319.0 [M+H]⁺.

### Synthesis of 1-(4-(2,2,2-trifluoro-1-hydroxyethyl)benzyl)-1H-pyrazole-4-carboxylic acid

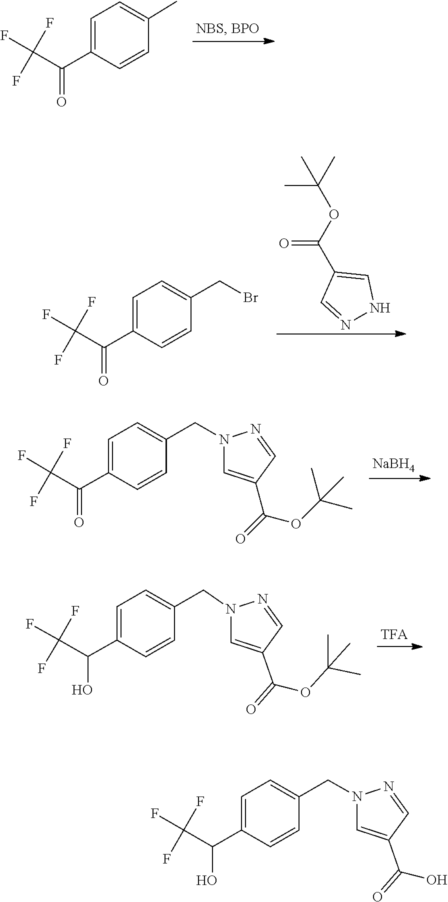

### Step 1: 1-(4-(bromomethyl)phenyl)-2,2,2-trifluoroethan-1-one

To a solution of 2,2,2-trifluoro-1-(p-tolyl)ethan-1-one (1 g, 5.31 mmol) in CCl₄ (12 mL) was added NBS (1.13 g, 6.38 mmol) and BPO (257 mg, 1.06 mmol). The reaction was heated at 80° C. overnight then diluted with water (30 mL) and extracted with DCM (50 mL×2). The combined organic layers were washed with brine, dried over Na₂SO₄, filtered and concentrated. The residue was purified by prep-TLC (eluent: Pet. ether:EtOAc=5:1, v/v) to afford 1-(4-(bromomethyl)phenyl)-2,2,2-trifluoroethan-1-one (800 mg, 57%) as a yellow oil. ¹H NMR (400 MHz, Chloroform-d) δ 8.06 (d, J=8.0 Hz, 2H), 7.57 (d, J=8.4, 2H), 4.51 (s, 2H).

### Step 2: tert-butyl 1-(4-(2,2,2-trifluoroacetyl)benzyl)-1H-pyrazole-4-carboxylate

To a solution of tert-butyl 1H-pyrazole-4-carboxylate (300 mg, 1.78 mmol) in DMF (4 mL) was added K₂CO₃ (739 mg, 5.35 mmol), KI (6 mg, 0.04 mmol) and 1-(4-(bromomethyl)phenyl)-2,2,2-trifluoroethan-1-one (600 mg, 1.78 mmol). The reaction mixture was stirred at room temperature for 3 h then was diluted with water (30 mL) and extracted with EtOAc (50 mL×2). The combined organic layers were washed with brine, dried over Na₂SO₄, filtered and concentrated. The residue was purified by prep-TLC (eluent: Petroleum ether:EtOAc=2:1, v/v) to afford tert-butyl 1-(4-(2,2,2-trifluoroacetyl)benzyl)-1H-pyrazole-4-carboxylate (350 mg, 44%) as a colorless oil. ¹H NMR (400 MHz, Chloroform-d) δ 8.06 (d, J=8.0 Hz, 2H), 7.89 (d, J=11.8 Hz, 2H), 7.35 (d, J=8.2 Hz, 2H), 5.40 (s, 2H), 1.54 (s, 9H).

### Step 3: tert-butyl 1-(4-(2,2,2-trifluoro-1-hydroxyethyl)benzyl)-1H-pyrazole-4-carboxylate

To a solution of tert-butyl 1-(4-(2,2,2-trifluoroacetyl)benzyl)-1H-pyrazole-4-carboxylate (300 mg, 0.85 mmol) in DCE (4 mL) at 0° C. was added NaBH₄ (40 mg, 1.02 mmol) and 15 drops of MeOH. The reaction was stirred at room temperature for 2 h then was diluted with water (30 mL) and extracted with DCM (50 mL×2). The combined organic layers were washed with brine, dried over Na₂SO₄, filtered and concentrated. The residue was purified by prep-TLC (eluent: Pet. ether:EtOAc=2:1, v/v) to afford tert-butyl 1-(4-(2,2,2-trifluoro-1-hydroxyethyl)benzyl)-1H-pyrazole-4-carboxylate (200 mg, 66%) as a colorless oil. LCMS m/z=357.1 [M+H]⁺; ¹H NMR (400 MHz, DMSO-d₆) δ 8.35 (s, 1H), 7.78 (s, 1H), 7.46 (d, J=8.0 Hz, 2H), 7.28 (d, J=8.0 Hz, 2H), 6.81 (d, J=5.4 Hz, 1H), 5.37 (s, 2H), 5.17-5.09 (m, 1H), 1.48 (s, 9H).

### Step 4: 1-(4-(2,2,2-trifluoro-1-hydroxyethyl)benzyl)-1H-pyrazole-4-carboxylic acid

To a solution of tert-butyl 1-(4-(2,2,2-trifluoro-1-hydroxyethyl)benzyl)-1H-pyrazole-4-carboxylate (90 mg, 0.25 mmol) in DCM (2 mL) was added TFA (1 mL) and the reaction stirred at room temperature for 1 h. The solvent was removed under vacuum to afford 1-(4-(2,2,2-trifluoro-1-hydroxyethyl)benzyl)-1H-pyrazole-4-carboxylic acid (76 mg, 100%) which was used directly in the next step. LCMS m/z=301.1 [M+H]⁺.

### Synthesis of 2-(4-cyclohexylphenyl)-2-(5-(6-(5-hydroxypyrazine-2-carbonyl)-2-(1-(trifluoromethyl)cyclopropane-1-carbonyl)-2,6-diazaspiro[3.4]octan-8-yl)-1,3,4-oxadiazol-2-yl)-1-(4-(thiazol-2-yl)piperidin-1-yl)ethan-1-one (I-109). (1021148)

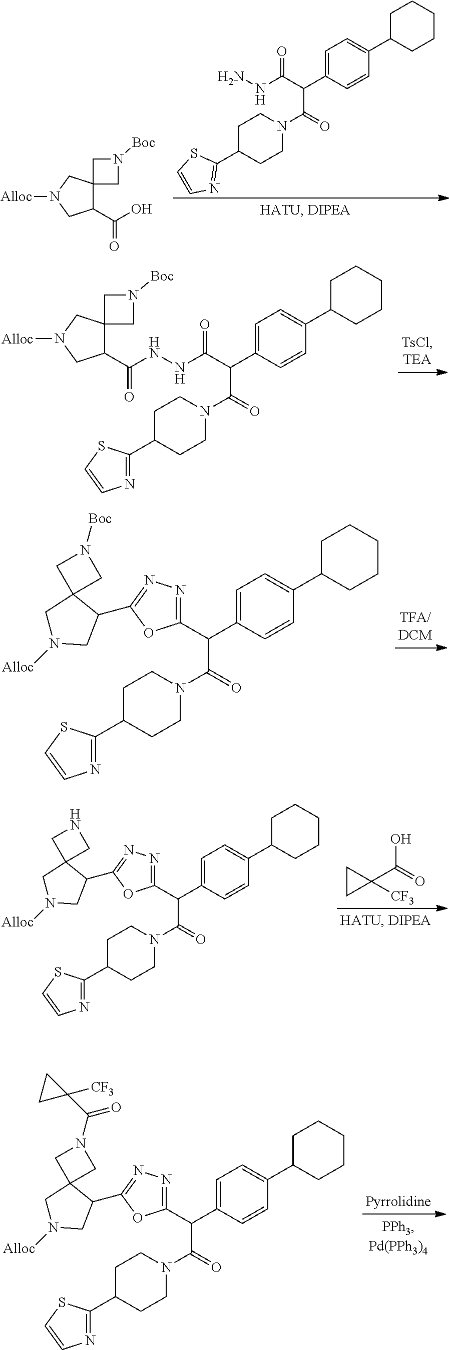

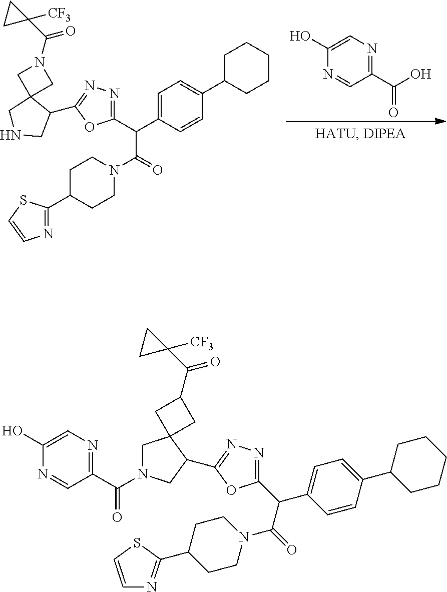 [SMILES: O=C(C(c1ccc(C2CCCCC2)cc1)c1nnc(C2CNCC23CN(C(=O)C2(C(F)(F)F)CC2)C3)o1)N1CCC(c2nccs2)CC1 ; O=C(O)c1cnc(O)cn1 ; O=C(c1cnc(O)cn1)N1CC(c2nnc(C(C(=O)N3CCC(c4nccs4)CC3)c3ccc(C4CCCCC4)cc3)o2)C2(CC(C(=O)C3(C(F)(F)F)CC3)C2)C1]

### Step 1: 6-allyl 2-(tert-butyl) 8-(2-(2-(4-cyclohexylphenyl)-3-oxo-3-(4-(thiazol-2-yl)piperidin-1-yl)propanoyl)hydrazine-1-carbonyl)-2,6-diazaspiro[3.4]octane-2,6-dicarboxylate

To a solution of 6-((allyloxy)carbonyl)-2-(tert-butoxycarbonyl)-2,6-diazaspiro[3.4]octane-8-carboxylic acid (862 mg, 2.53 mmol) in DCM (8 mL) was added HATU (964 mg, 2.53 mmol) and DIPEA (819 mg, 6.33 mmol) and the mixture stirred at room temperature for 20 mins. 2-(4-cyclohexylphenyl)-3-oxo-3-(4-(thiazol-2-yl)piperidin-1-yl)propanehydrazide (900 mg, 2.11 mmol) was added and stirring continued for 2 h. The reaction was diluted with water (40 mL) and extracted with DCM (40 mL×3). The combined organic layers were washed with brine, dried over Na₂SO₄, filtered and concentrated. The residue obtained was purified by RP column (eluent: MeCN:H₂O=55%:45%) to afford 6-allyl 2-(tert-butyl) 8-(2-(2-(4-cyclohexylphenyl)-3-oxo-3-(4-(thiazol-2-yl)piperidin-1-yl)propanoyl)hydrazine-1-carbonyl)-2,6-diazaspiro[3.4]octane-2,6-dicarboxylate (300 mg, 19%) as a white solid. LCMS m/z=749.2 [M+H]⁺; ¹H NMR (400 MHz, DMSO-d6) δ 10.31-10.09 (m, 2H), 7.73-7.66 (m, 1H), 7.58 (dd, J=7.6, 4.4 Hz, 1H), 7.23 (t, J=8.2 Hz, 2H), 7.18-7.15 (m, 2H), 5.91 (m, 1H), 5.27 (m, 1H), 5.16 (s, 1H), 4.97 (s, 1H), 4.49 (d, J=5.2 Hz, 2H), 4.46-4.35 (m, 1H), 3.97 (d, J=8.4 Hz, 1H), 3.81-3.41 (m, 9H), 3.27-3.13 (m, 2H), 3.08 (m, 1H), 2.77 (m, 1H), 2.19 (m, 1H), 2.04 (m, 1H), 1.72 (m, 7H), 1.44-1.37 (m, 5H), 1.35 (s, 9H).

### Step 2: 6-allyl 2-(tert-butyl) 8-(5-(1-(4-cyclohexylphenyl)-2-oxo-2-(4-(thiazol-2-yl)piperidin-1-yl)ethyl)-1,3,4-oxadiazol-2-yl)-2,6-diazaspiro[3.4]octane-2,6-dicarboxylate

To a solution of 6-allyl 2-(tert-butyl) 8-(2-(2-(4-cyclohexylphenyl)-3-oxo-3-(4-(thiazol-2-yl)piperidin-1-yl)propanoyl)hydrazine-1-carbonyl)-2,6-diazaspiro[3.4]octane-2,6-dicarboxylate (50 mg, 0.07 mmol) in DCM (1 mL) was added TEA (33.8 mg, 0.33 mmol) and TsCl (38.2 mg, 0.20 mmol). The reaction was stirred at room temperature for 1 h then the solvent was removed under vacuum and the residue obtained purified by prep-TLC (eluent: DCM:MeOH=20:1) to afford 6-allyl 2-(tert-butyl) 8-(5-(1-(4-cyclohexylphenyl)-2-oxo-2-(4-(thiazol-2-yl)piperidin-1-yl)ethyl)-1,3,4-oxadiazol-2-yl)-2,6-diazaspiro[3.4]octane-2,6-dicarboxylate (20 mg, 41%) as a white solid. LCMS m/z=731.2 [M+H]⁺.

### Step 3: allyl 8-(5-(1-(4-cyclohexylphenyl)-2-oxo-2-(4-(thiazol-2-yl)piperidin-1-yl)ethyl)-1,3,4-oxadiazol-2-yl)-2,6-diazaspiro[3.4]octane-6-carboxylate

To a solution of 6-allyl 2-(tert-butyl) 8-(5-(1-(4-cyclohexylphenyl)-2-oxo-2-(4-(thiazol-2-yl)piperidin-1-yl)ethyl)-1,3,4-oxadiazol-2-yl)-2,6-diazaspiro[3.4]octane-2,6-dicarboxylate (200 mg, 0.27 mmol) in DCM (2 mL) was added TFA (1 mL) and the reaction mixture stirred at room temperature for 2 h. The solvent was removed under vacuum to afford allyl 8-(5-(1-(4-cyclohexylphenyl)-2-oxo-2-(4-(thiazol-2-yl)piperidin-1-yl)ethyl)-1,3,4-oxadiazol-2-yl)-2,6-diazaspiro[3.4]octane-6-carboxylate (170 mg, 100%) which was used directly in the next step. LCMS m/z=631.3 [M+H]⁺.

### Step 4: allyl 8-(5-(1-(4-cyclohexylphenyl)-2-oxo-2-(4-(thiazol-2-yl)piperidin-1-yl)ethyl)-1,3,4-oxadiazol-2-yl)-2-(1-(trifluoromethyl)cyclopropane-1-carbonyl)-2,6-diazaspiro[3.4]octane-6-carboxylate

To a solution of 1-(trifluoromethyl)cyclopropane-1-carboxylic acid (51 mg, 0.33 mmol) in DCM (3 mL) was added HATU (125 mg, 0.33 mmol) and DIPEA (106 mg, 0.82 mmol) and the mixture stirred at room temperature for 20 mins. Allyl 8-(5-(1-(4-cyclohexylphenyl)-2-oxo-2-(4-(thiazol-2-yl)piperidin-1-yl)ethyl)-1,3,4-oxadiazol-2-yl)-2,6-diazaspiro[3.4]octane-6-carboxylate (173 mg, 0.27 mmol) was added and stirring continued for 2 h. The mixture was diluted with water (15 mL) and extracted with DCM (15 mL×3). The combined organic layers were washed with brine, dried over Na₂SO₄, filtered and concentrated. The residue obtained was purified by prep-TLC (eluent: Pet.Ether:EtOAc=1:1) to afford allyl 8-(5-(1-(4-cyclohexylphenyl)-2-oxo-2-(4-(thiazol-2-yl)piperidin-1-yl)ethyl)-1,3,4-oxadiazol-2-yl)-2-(1-(trifluoromethyl)cyclopropane-1-carbonyl)-2,6-diazaspiro[3.4]octane-6-carboxylate (180 mg, 86%) as a colorless oil. LCMS m/z=767.2 [M+H]⁺; ¹H NMR (400 MHz, DMSO-d6) δ 7.75-7.53 (m, 2H), 7.40-7.30 (m, 2H), 7.25 (dd, J=8.0, 4.4 Hz, 2H), 6.06-5.85 (m, 2H), 5.30 (m, 1H), 5.18 (d, J=10.4 Hz, 1H), 4.53 (d, J=5.2 Hz, 2H), 3.92 (d, J=14.0 Hz, 1H), 3.78-3.59 (m, 4H), 3.51-3.44 (m, 4H), 3.43-3.35 (m, 4H), 3.20 (m, 1H), 2.81 (t, J=12.2 Hz, 1H), 1.74 (m, 5H), 1.49-1.22 (m, 11H), 1.12 (s, 2H).

### Step 5: 2-(4-cyclohexylphenyl)-1-(4-(thiazol-2-yl)piperidin-1-yl)-2-(5-(2-(1-(trifluoromethyl)cyclopropane-1-carbonyl)-2,6-diazaspiro[3.4]octan-8-yl)-1,3,4-oxadiazol-2-yl)ethan-1-one

To a solution of allyl 8-(5-(1-(4-cyclohexylphenyl)-2-oxo-2-(4-(thiazol-2-yl)piperidin-1-yl)ethyl)-1,3,4-oxadiazol-2-yl)-2-(1-(trifluoromethyl)cyclopropane-1-carbonyl)-2,6-diazaspiro[3.4]octane-6-carboxylate (100 mg, 0.13 mmol) in DCM (1.5 mL) at 0° C. was added PPh₃ (8.6 mg, 0.033 mmol) and Pd(PPh₃)₄ (15.1 mg, 0.013 mmol) and the mixture was stirred for 20 mins. Pyrrolidine (11 mg, 0.16 mmol) was added and the reaction allowed to warm to room temperature and stirred for another 1 h. The mixture was diluted with water (5 mL) and extracted with DCM (5 mL×3). The combined organic layers were washed with brine, dried over Na₂SO₄, filtered and concentrated to afford 2-(4-cyclohexylphenyl)-1-(4-(thiazol-2-yl)piperidin-1-yl)-2-(5-(2-(1-(trifluoromethyl)cyclopropane-1-carbonyl)-2,6-diazaspiro[3.4]octan-8-yl)-1,3,4-oxadiazol-2-yl)ethan-1-one (90 mg, 100%) as a colorless oil which was used directly in the next step. LCMS m/z=683.2 [M+H]⁺.

### Step 6: 2-(4-cyclohexylphenyl)-2-(5-(6-(5-hydroxypyrazine-2-carbonyl)-2-(1-(trifluoromethyl)cyclopropane-1-carbonyl)-2,6-diazaspiro[3.4]octan-8-yl)-1,3,4-oxadiazol-2-yl)-1-(4-(thiazol-2-yl)piperidin-1-yl)ethan-1-one

To a solution of 5-hydroxypyrazine-2-carboxylic acid (18.3 mg, 0.13 mmol) in DCM (2 mL) was added HATU (50 mg, 0.13 mmol) and DIPEA (51 mg, 0.39 mmol) and the mixture stirred at room temperature for 20 mins. Allyl 8-(5-(1-(4-cyclohexylphenyl)-2-oxo-2-(4-(thiazol-2-yl)piperidin-1-yl)ethyl)-1,3,4-oxadiazol-2-yl)-2-(1-(trifluoromethyl)cyclopropane-1-carbonyl)-2,6-diazaspiro[3.4]octane-6-carboxylate (89 mg, 0.13 mmol) was added and stirring continued for 2 h. The mixture was diluted with water (15 mL) and extracted with DCM (15 mL×3). The combined organic layers were washed with brine, dried over Na₂SO₄, filtered and concentrated. The residue obtained was purified by prep-HPLC to afford 2-(4-cyclohexylphenyl)-2-(5-(6-(5-hydroxypyrazine-2-carbonyl)-2-(1-(trifluoromethyl)cyclopropane-1-carbonyl)-2,6-diazaspiro[3.4]octan-8-yl)-1,3,4-oxadiazol-2-yl)-1-(4-(thiazol-2-yl)piperidin-1-yl)ethan-1-one (6.4 mg, 6%) as a white solid. LCMS m/z=805.3 [M+H]⁺; ¹H NMR (400 MHz, Chloroform-d) δ 8.09 (d, J=17.0 Hz, 2H), 7.74-7.64 (m, 1H), 7.32 (s, 3H), 7.21 (s, 2H), 5.54 (s, 1H), 4.61-3.71 (m, 12H), 3.21 (s, 1H), 2.99-2.86 (m, 1H), 2.50 (s, 1H), 2.08 (m, 3H), 1.80-1.60 (m, 7H), 1.38 (d, J=10.0 Hz, 4H), 1.26-1.16 (m, 4H).

### Synthesis of 2-(4-(tetrahydro-2H-pyran-4-yl)phenyl)-1-(4-(thiazol-2-yl)piperidin-1-yl)-2-(5-(6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)-1,3,4-oxadiazol-2-yl)ethan-1-one (I-216)

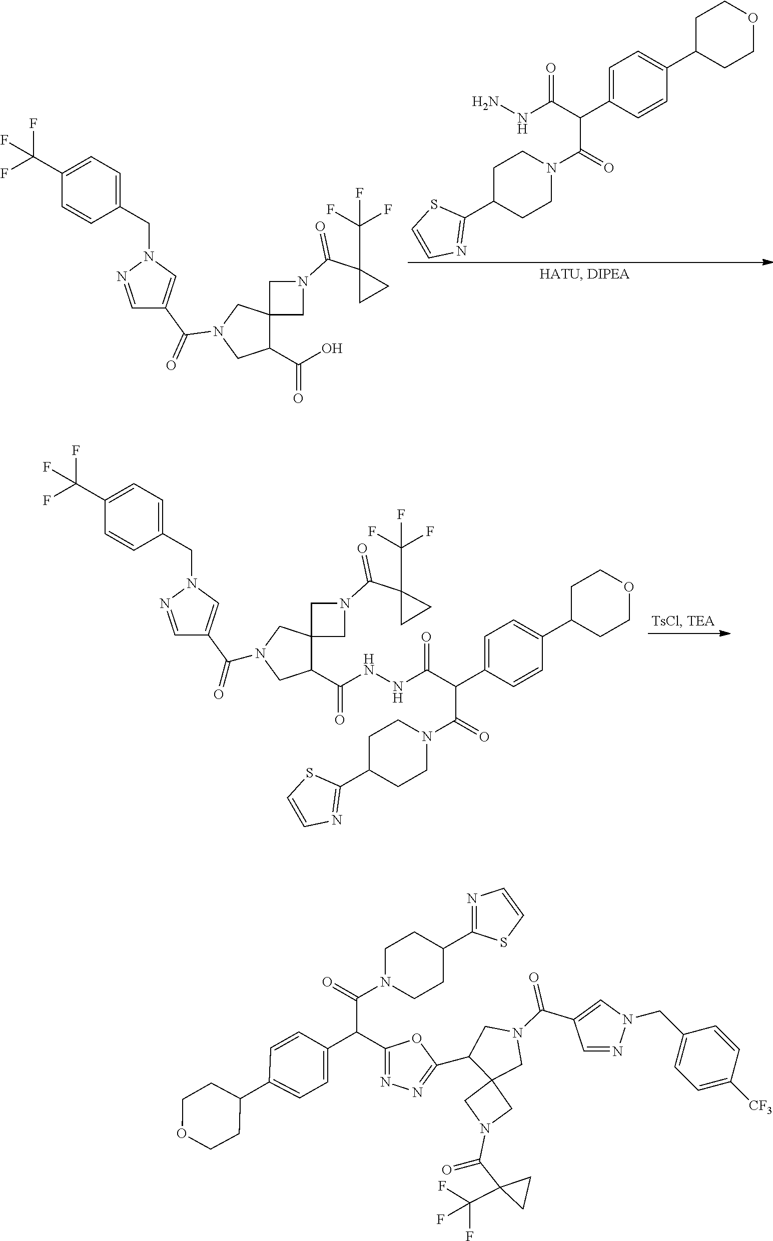

### Step 1: N′-(3-oxo-2-(4-(tetrahydro-2H-pyran-4-yl)phenyl)-3-(4-(thiazol-2-yl)piperidin-1-yl)propanoyl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbohydrazide

To a solution of 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (100 mg, 0.18 mmol) in DCM (4 mL) was added HATU (70 mg, 0.18 mmol) and DIPEA (71 mg, 0.55 mol) and the mixture stirred at room temperature for 20 mins. 3-oxo-2-(4-(tetrahydro-2H-pyran-4-yl)phenyl)-3-(4-(thiazol-2-yl)piperidin-1-yl)propanehydrazide (78.7 mg, 0.18 mmol) was added and stirring continued for 2 h. The mixture was diluted with water (20 mL) and extracted with DCM (20 mL×3). The combined organic layers were washed with brine, dried over Na₂SO₄, filtered and concentrated. The residue obtained was purified by prep-TLC (eluent: DCM:MeOH=15:1) to afford N′-(3-oxo-2-(4-(tetrahydro-2H-pyran-4-yl)phenyl)-3-(4-(thiazol-2-yl)piperidin-1-yl)propanoyl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbohydrazide (40 mg, 23%) as a white solid. LCMS m/z=955.2 [M+H]⁺.

### Step 2: 2-(4-(tetrahydro-2H-pyran-4-yl)phenyl)-1-(4-(thiazol-2-yl)piperidin-1-yl)-2-(5-(6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)-1,3,4-oxadiazol-2-yl)ethan-1-one

To a solution of N′-(3-oxo-2-(4-(tetrahydro-2H-pyran-4-yl)phenyl)-3-(4-(thiazol-2-yl)piperidin-1-yl)propanoyl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbohydrazide (230 mg, 0.24 mmol) in DCM (5 mL) was added TEA (122 mg, 1.21 mmol), and TsCl (138 mg, 0.72 mmol). The reaction was stirred at room temperature for 3 h then the solvent was removed under reduced pressure. The residue obtained was purified by prep-HPLC to afford 2-(4-(tetrahydro-2H-pyran-4-yl)phenyl)-1-(4-(thiazol-2-yl)piperidin-1-yl)-2-(5-(6-(1-(4-(trifluoromethyl)benzyl)-1H-pyrazole-4-carbonyl)-2-(1-(trifluoromethyl)cyclopropane-1-carbonyl)-2,6-diazaspiro[3.4]octan-8-yl)-1,3,4-oxadiazol-2-yl)ethan-1-one (100 mg, 44%) as a white solid. LCMS m/z=937.2 [M+H]⁺; ¹H NMR (400 MHz, Chloroform-d) δ 7.86 (m, 2H), 7.66-7.58 (m, 3H), 7.34 (d, J=7.8 Hz, 4H), 7.28 (s, 1H), 7.24 (s, 2H), 5.53 (m, 1H), 5.38 (s, 2H), 4.64-4.46 (m, 1H), 4.20-3.73 (m, 11H), 3.51 (s, 2H), 3.20 (d, J=13.0 Hz, 2H), 3.05-2.65 (m, 3H), 2.21-1.97 (m, 2H), 1.80 (m, 6H), 1.18 (m, 4H).

### Synthesis of (1-benzyl-1H-pyrazol-4-yl)(8-(4-(benzylamino)phenyl)-2-((S)-2,2-dimethylcyclopropane-1-carbonyl)-2,6-diazaspiro[3.4]octan-6-yl)methanone (I-93)

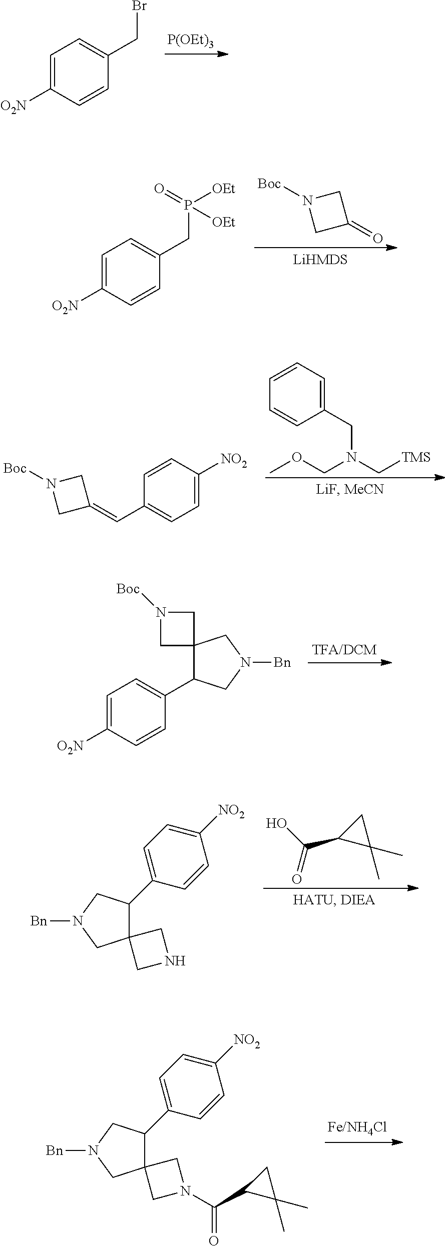

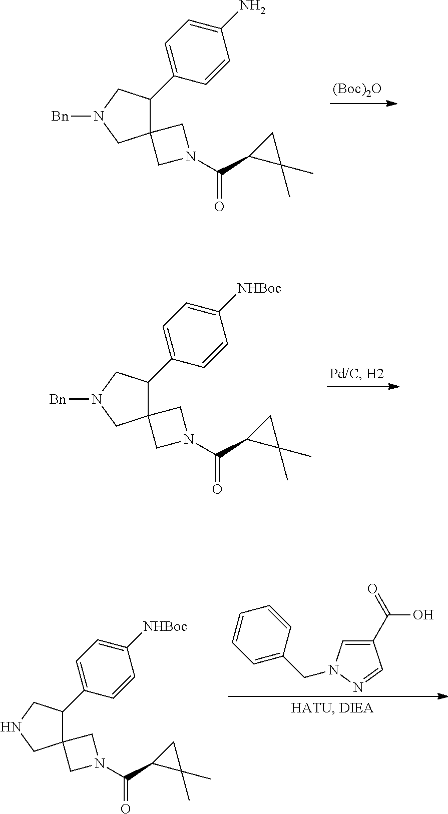 [SMILES: "(Boc)2O" ; CC1(C)C[C@@H]1C(=O)N1CC2(CN(Cc3ccccc3)CC2c2ccc(N)cc2)C1 ; CC(C)(C)OC(=O)Nc1ccc(C2CN(Cc3ccccc3)CC23CN(C(=O)[C@H]2CC2(C)C)C3)cc1 ; CC(C)(C)OC(=O)Nc1ccc(C2CNCC23CN(C(=O)[C@H]2CC2(C)C)C3)cc1 ; O=C(O)c1cnn(Cc2ccccc2)c1]

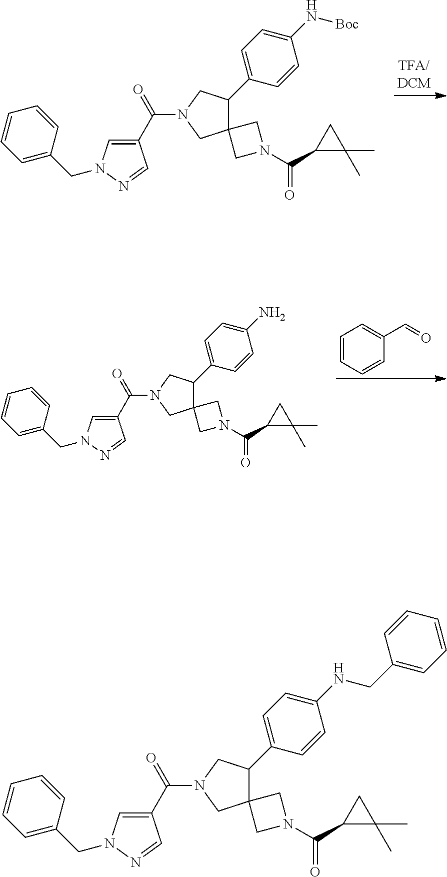 [SMILES: CC(C)(C)OC(=O)Nc1ccc(C2CN(C(=O)c3cnn(Cc4ccccc4)c3)CC23CN(C(=O)[C@H]2CC2(C)C)C3)cc1 ; CC1(C)C[C@@H]1C(=O)N1CC2(CN(C(=O)c3cnn(Cc4ccccc4)c3)CC2c2ccc(N)cc2)C1 ; O=Cc1ccccc1 ; CC1(C)C[C@@H]1C(=O)N1CC2(CN(C(=O)c3cnn(Cc4ccccc4)c3)CC2c2ccc(NCc3ccccc3)cc2)C1]

### Step 1: diethyl (4-nitrobenzyl)phosphonate

A solution of 1-(bromomethyl)-4-nitrobenzene (5 g, 23.14 mmol) in P(OEt)₃ (20 mL) was heated at 50° C. overnight. The solvent was removed under vacuum to afford diethyl (4-nitrobenzyl)phosphonate (4 g, 63%) as a red oil which was used directly in the next step. ¹H NMR (400 MHz, DMSO-d₆) δ 8.21-8.14 (m, 2H), 7.56 (m, 2H), 4.02-3.96 (m, 4H), 3.48 (s, 1H), 3.43 (s, 1H), 1.17 (t, J=7.2 Hz, 6H).

### Step 2: tert-butyl 3-(4-nitrobenzylidene)azetidine-1-carboxylate

To a solution of diethyl (4-nitrobenzyl)phosphonate (1 g, 3.66 mol) and tert-butyl 3-oxoazetidine-1-carboxylate (940 mg, 5.49 mol) in anhydrous THF (5 mL) at 0° C. under a N₂ atmosphere was added LiHMDS (1.0 mol in THF) (4.4 mL, 4.39 mmol). The reaction was allowed to warm to room temperature and for 2 h then was diluted with water (100 mL) and extracted with EtOAc (100 mL×2). The combined organic layers were washed with brine, dried over Na₂SO₄, filtered and concentrated. The residue was purified by silica gel column (eluent: DCM:MeOH=80:1) to afford tert-butyl 3-(4-nitrobenzylidene)azetidine-1-carboxylate (900 mg, 84%) as a yellow solid. ¹H NMR (400 MHz, DMSO-d₆) δ 8.19 (d, J=8.8 Hz, 2H), 7.43 (d, J=9.0 Hz, 2H), 6.54 (t, J=2.4 Hz, 1H), 4.87 (s, 2H), 4.62 (s, 2H), 1.42 (s, 9H).

### Step 3: tert-butyl 6-benzyl-8-(4-nitrophenyl)-2,6-diazaspiro[3.4]octane-2-carboxylate

To a solution of tert-butyl 3-(4-nitrobenzylidene)azetidine-1-carboxylate (1 g, 3.45 mmol) in MeCN (10 mL) was added LiF (268 mg, 10.34 mmol) and the reaction heated at 80° C. overnight. The mixture was diluted with water (100 mL) and extracted with EtOAc (100 mL×2). The combined organic layers were washed with brine, dried over Na₂SO₄, filtered and concentrated. The residue obtained was purified by prep-TLC (eluent: Pet.Ether:EtOAc=3:1) to afford tert-butyl 6-benzyl-8-(4-nitrophenyl)-2,6-diazaspiro[3.4]octane-2-carboxylate (180 mg, 12%) as a red oil. LCMS m/z=424.2 [M+H]⁺; ¹H NMR (400 MHz, Methanol-d₄) δ 8.21 (d, J=8.8 Hz, 2H), 7.52 (d, J=8.8 Hz, 2H), 7.39-7.31 (m, 4H), 7.28-7.24 (m, 1H), 3.98-3.87 (m, 2H), 3.72 (d, J=3.2 Hz, 2H), 3.52 (t, J=7.6 Hz, 2H), 3.28 (s, 1H), 3.12 (m, 1H), 3.07-2.97 (m, 2H), 2.76 (m, 1H), 1.33 (s, 9H).

### Step 4: 6-benzyl-8-(4-nitrophenyl)-2,6-diazaspiro[3.4]octane

To a solution of tert-butyl 6-benzyl-8-(4-nitrophenyl)-2,6-diazaspiro[3.4]octane-2-carboxylate (100 mg, 0.24 mmol) in DCM (2 mL) was added TFA (1 mL) and the reaction stirred at room temperature for 1 h. The solvent was removed under vacuum to afford crude 6-benzyl-8-(4-nitrophenyl)-2,6-diazaspiro[3.4]octane (75 mg, 100%) as a red oil which was used directly in the next step. LCMS m/z=324.2 [M+H]⁺.

### Step 5: (6-benzyl-8-(4-nitrophenyl)-2,6-diazaspiro[3.4]octan-2-yl)((S)-2,2-dimethylcyclopropyl)methanone

To a solution of (S)-2,2-dimethylcyclopropane-1-carboxylic acid (32 mg, 0.28 mmol) in DCM (2 mL) was added HATU (132 mg, 0.35 mmol) and DIPEA (90 mg, 0.70 mmol) and the mixture stirred at room temperature for 20 min. 6-benzyl-8-(4-nitrophenyl)-2,6-diazaspiro[3.4]octane (75 mg, 0.23 mmol) was added and stirring continued for 2 h. The mixture was diluted with water (15 mL) and extracted with DCM (15 mL×2). The combined organic layers were washed with brine, dried over Na₂SO₄, filtered and concentrated. The residue obtained was purified by prep-TLC (eluent: DCM:MeOH=20:1) to afford (6-benzyl-8-(4-nitrophenyl)-2,6-diazaspiro[3.4]octan-2-yl)((S)-2,2-dimethylcyclopropyl)methanone (105 mg, 24%) as a red oil. LCMS m/z=420.2 [M+H]⁺; ¹H NMR (400 MHz, DMSO-d₆) δ 8.22 (td, J=8.2, 5.2 Hz, 2H), 7.63-7.56 (m, 2H), 7.39-7.31 (m, 4H), 7.28-7.23 (m, 1H), 4.08-3.90 (m, 2H), 3.87-3.77 (m, 1H), 3.69 (s, 2H), 3.61 (m, 1H), 3.52-3.34 (m, 2H), 2.97 (m, 3H), 1.41-1.38 (m, 1H), 1.25-1.22 (m, 2H), 1.14 (s, 3H), 1.10 (s, 3H).

### Step 6: (8-(4-aminophenyl)-6-benzyl-2,6-diazaspiro[3.4]octan-2-yl)((S)-2,2-dimethylcyclopropyl)methanone

To a suspension of (6-benzyl-8-(4-nitrophenyl)-2,6-diazaspiro[3.4]octan-2-yl)((S)-2,2-dimethylcyclopropyl)methanone (420 mg, 1.00 mmol) and Fe powder (285 mg, 5.01 mmol) in EtOH (6 mL) was added saturated aqueous NH₄Cl (2 mL) and the reaction heated at 80° C. for 1 h. The solvent was removed under vacuum and the residue obtained resuspendened in water (30 mL) and extracted with EtOAc (30 mL×2). The combined organic layers were washed with brine, dried over Na₂SO₄, filtered and concentrated. The residue obtained was purified by prep-TLC (eluent: DCM:MeOH=15:1) to afford (8-(4-aminophenyl)-6-benzyl-2,6-diazaspiro[3.4]octan-2-yl)((S)-2,2-dimethylcyclopropyl)methanone (220 mg, 56%) as a yellow sold. LCMS m/z=390.2 [M+H]⁺.

### Step 7: tert-butyl (4-(6-benzyl-2-((S)-2,2-dimethylcyclopropane-1-carbonyl)-2,6-diazaspiro[3.4]octan-8-yl)phenyl)carbamate

To a solution of (8-(4-aminophenyl)-6-benzyl-2,6-diazaspiro[3.4]octan-2-yl)((S)-2,2-dimethylcyclopropyl)methanone (230 mg, 0.59 mmol) in a mixture of THF and 1.0 M NaOH (0.5 mL/0.5 mL) was added (Boc)₂O (142 mg, 0.65 mmol) and the reaction stirred at room temperature for 2 h. The mixture was diluted with water (30 mL) and extracted with EtOAc (30 mL×2). The combined organic layers were washed with brine, dried over Na₂SO₄, filtered and concentrated to afford tert-butyl (4-(6-benzyl-2-((S)-2,2-dimethylcyclopropane-1-carbonyl)-2,6-diazaspiro[3.4]octan-8-yl)phenyl)carbamate (200 mg, 69%) as a yellow oil. LCMS m/z=490.3 [M+H]f; ¹H NMR (400 MHz, DMSO-d6) δ 9.29 (d, J=9.8 Hz, 1H), 7.44-7.30 (m, 6H), 7.25 (m, 1H), 7.13 (q, J=8.4 Hz, 2H), 4.21-3.96 (m, 1H), 3.86-3.60 (m, 4H), 3.47-3.33 (m, 1H), 3.19 (dd, J=9.6, 7.6 Hz, 1H), 3.06-2.79 (m, 3H), 2.63 (m, 1H), 1.47 (s, 9H), 1.13-1.01 (m, 3H), 0.97-0.86 (m, 3H), 0.75 (m, 1H), 0.71 (s, 1H), 0.57 (m, 1H).

### Step 8: tert-butyl (4-(2-((S)-2,2-dimethylcyclopropane-1-carbonyl)-2,6-diazaspiro[3.4]octan-8-yl)phenyl)carbamate

To a solution of tert-butyl (4-(6-benzyl-2-((S)-2,2-dimethylcyclopropane-1-carbonyl)-2,6-diazaspiro[3.4]octan-8-yl)phenyl)carbamate (10 mg, 0.02 mmol) in a mixture of IPrOH and THF (0.5 mL/0.5 mL) was added 10% Pd/C (4 mg) and the reaction stirred under a H₂ atmosphere for 4 h. The catalyst was removed by filtration through Celite and the filtrate concentrated to afford tert-butyl (4-(2-((S)-2,2-dimethylcyclopropane-1-carbonyl)-2,6-diazaspiro[3.4]octan-8-yl)phenyl)carbamate (8 mg, 100%) as a brown oil. LCMS m/z=400.2 [M+H]f.

### Step 9: tert-butyl (4-(6-(1-benzyl-1H-pyrazole-4-carbonyl)-2-((S)-2,2-dimethylcyclopropane-1-carbonyl)-2,6-diazaspiro[3.4]octan-8-yl)phenyl)carbamate

To a solution of 1-benzyl-1H-pyrazole-4-carboxylic acid (150 mg, 0.74 mmol) in DCM (2 mL) was added HATU (282 mg, 0.74 mmol) and DIPEA (288 mg, 2.23 mmol) and the mixture stirred at room temperature for 20 mins. Tert-butyl (4-(2-((S)-2,2-dimethylcyclopropane-1-carbonyl)-2,6-diazaspiro[3.4]octan-8-yl)phenyl)carbamate (356 mg, 0.89 mmol) was added and stirring continued for 2 h. The mixture was diluted with water (30 mL) and extracted with DCM (30 mL×2). The combined organic layers were washed with brine, dried over Na₂SO₄, filtered and concentrated. The residue obtained was purified by prep-TLC (eluent: DCM:MeOH=20:1) to afford tert-butyl (4-(6-(1-benzyl-1H-pyrazole-4-carbonyl)-2-((S)-2,2-dimethylcyclopropane-1-carbonyl)-2,6-diazaspiro[3.4]octan-8-yl)phenyl)carbamate (105 mg, 24%) as a yellow solid. LCMS m/z=584.3 [M+H]⁺.

### Step 10: (8-(4-aminophenyl)-2-((S)-2,2-dimethylcyclopropane-1-carbonyl)-2,6-diazaspiro[3.4]octan-6-yl)(1-benzyl-1H-pyrazol-4-yl)methanone

To a solution of tert-butyl (4-(6-(1-benzyl-1H-pyrazole-4-carbonyl)-2-((S)-2,2-dimethylcyclopropane-1-carbonyl)-2,6-diazaspiro[3.4]octan-8-yl)phenyl)carbamate (100 mg, 0.17 mmol) in DCM (2 mL) was added TFA (1 mL) and the reaction stirred at room temperature for 2 h. The solvent was removed under vacuum to afford (8-(4-aminophenyl)-2-((S)-2,2-dimethylcyclopropane-1-carbonyl)-2,6-diazaspiro[3.4]octan-6-yl)(1-benzyl-1H-pyrazol-4-yl)methanone (83 mg, 100%) as a red oil which was used directly in the next step. LCMS m/z=484.3 [M+H]⁺.

### Step 11: (1-benzyl-1H-pyrazol-4-yl)(8-(4-(benzylamino)phenyl)-2-((S)-2,2-dimethylcyclopropane-1-carbonyl)-2,6-diazaspiro[3.4]octan-6-yl)methanone

To a solution of (8-(4-aminophenyl)-2-((S)-2,2-dimethylcyclopropane-1-carbonyl)-2,6-diazaspiro[3.4]octan-6-yl)(1-benzyl-1H-pyrazol-4-yl)methanone (82 mg, 0.17 mmol) in toluene (4 mL) was added TEA (0.5 mL×2), benzaldehyde (36 mg, 0.34 mmol), AcOH (1 mL) and titanium tetraisopropanolate (482 mg, 1.70 mmol). The mixture was heated at reflux overnight then was cooled to room temperature. NaBH₄ (26 mg, 0.68 mmol) was added and stirring continued for 4 h. The mixture was diluted with water (30 mL) and extracted with DCM (30 mL×2). The combined organic layers were washed with brine, dried over Na₂SO₄, filtered and concentrated. The residue obtained was purified by silica gel column (eluent: DCM:MeOH=10:1) and prep-TLC (eluent: DCM:MeOH=20:1) to afford (1-benzyl-1H-pyrazol-4-yl)(8-(4-(benzylamino)phenyl)-2-((S)-2,2-dimethylcyclopropane-1-carbonyl)-2,6-diazaspiro[3.4]octan-6-yl)methanone (25 mg, 26%) as a white solid. LCMS m/z=574.3 [M+H]⁺; ¹H NMR (400 MHz, Methanol-d₄) δ 8.25 (d, J=11.8 Hz, 1H), 7.96 (d, J=10.8 Hz, 1H), 7.37-7.25 (m, 9H), 7.23-7.17 (m, 1H), 7.05 (m, 2H), 6.63 (d, J=7.8 Hz, 2H), 5.38 (d, J=9.8 Hz, 2H), 4.30 (s, 2H), 4.27-3.79 (m, 8H), 3.50-3.34 (m, 1H), 1.31 (m, 1H), 1.17-1.02 (m, 4H), 0.94 (m, 1H), 0.82 (s, 1H), 0.72-0.59 (m, 2H).

### Synthesis of N-(4-(6-(1-benzyl-1H-pyrazole-4-carbonyl)-2-((S)-2,2-dimethylcyclopropane-1-carbonyl)-2,6-diazaspiro[3.4]octan-8-yl)phenyl)cyclohexanecarboxamide (I-87)

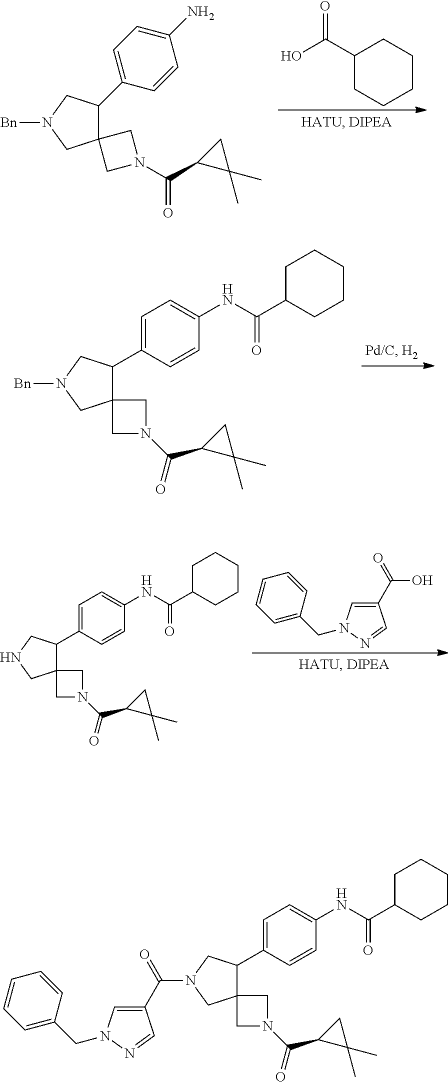

### Step 1: N-(4-(6-benzyl-2-((S)-2,2-dimethylcyclopropane-1-carbonyl)-2,6-diazaspiro[3.4]octan-8-yl)phenyl)cyclohexanecarboxamide

To a solution of cyclohexanecarboxylic acid (18 mg, 0.14 mmol) in DCM (2 mL) was added HATU (64 mg, 0.17 mmol) and DIPEA (42 mg, 0.32 mmol) and the mixture stirred at room temperature for 20 min. (8-(4-aminophenyl)-6-benzyl-2,6-diazaspiro[3.4]octan-2-yl)((S)-2,2-dimethylcyclopropyl)methanone (50 mg, 0.13 mmol) was added and stirring continued for 2 h. The mixture was diluted with water (5 mL) and extracted with DCM (5 mL×2). The combined organic layers were washed with brine, dried over Na₂SO₄, filtered and concentrated. The residue obtained was purified by prep-TLC (eluent: DCM:MeOH=20:1) to afford N-(4-(6-benzyl-2-((S)-2,2-dimethylcyclopropane-1-carbonyl)-2,6-diazaspiro[3.4]octan-8-yl)phenyl)cyclohexanecarboxamide (27 mg, 42%) as a yellow solid. LCMS m/z=500.3 [M+H]⁺; ¹H NMR (400 MHz, DMSO-d6) δ 9.76 (d, J=5.8 Hz, 1H), 7.61-7.51 (m, 2H), 7.35 (d, J=5.6 Hz, 4H), 7.26 (s, 1H), 7.16 (q, J=8.0 Hz, 2H), 4.21-4.02 (m, 1H), 3.78-3.54 (m, 4H), 3.04-2.83 (m, 3H), 2.30 (m, 1H), 1.75 (d, J=12.2 Hz, 5H), 1.68-1.62 (m, 2H), 1.43-1.32 (m, 3H), 1.28 (m, 3H), 1.10 (m, 2H), 1.02-0.95 (m, 2H), 0.87 (m, 2H), 0.78-0.67 (m, 2H), 0.63-0.50 (m, 1H).

### Step 2: N-(4-(2-((S)-2,2-dimethylcyclopropane-1-carbonyl)-2,6-diazaspiro[3.4]octan-8-yl)phenyl)cyclohexanecarboxamide

To a solution of N-(4-(6-benzyl-2-((S)-2,2-dimethylcyclopropane-1-carbonyl)-2,6-diazaspiro[3.4]octan-8-yl)phenyl)cyclohexanecarboxamide (100 mg, 0.20 mmol) in EtOAc (4 mL) was added 10% Pd/C (40 mg) and the reaction stirred under a H₂ atmosphere for 24 h. The catalyst was removed by filtration through Celite and the filtrate concentrated to afford N-(4-(2-((S)-2,2-dimethylcyclopropane-1-carbonyl)-2,6-diazaspiro[3.4]octan-8-yl)phenyl)cyclohexanecarboxamide (81 mg, 100%) as a yellow oil. LCMS m/z=410.3 [M+H]⁺.

### Step 3: N-(4-(6-(1-benzyl-1H-pyrazole-4-carbonyl)-2-((S)-2,2-dimethylcyclopropane-1-carbonyl)-2,6-diazaspiro[3.4]octan-8-yl)phenyl)cyclohexanecarboxamide

To a solution of 1-benzyl-1H-pyrazole-4-carboxylic acid (50 mg, 0.25 mmol) in DCM (2 mL) was added HATU (94 mg, 0.25 mmol) and DIPEA (96 mg, 0.74 mmol) and the mixture was stirred for 20 min. N-(4-(2-((S)-2,2-dimethylcyclopropane-1-carbonyl)-2,6-diazaspiro[3.4]octan-8-yl)phenyl)cyclohexanecarboxamide (121.5 mg, 0.29 mmol) was added and stirring continued for 2 h. The mixture was diluted with water (10 mL) and extracted with DCM (10 mL×2). The combined organic layers were washed with brine, dried over Na₂SO₄, filtered and concentrated. The residue obtained was purified by prep-TLC (eluent: DCM:MeOH=20:1) to afford N-(4-(6-(1-benzyl-1H-pyrazole-4-carbonyl)-2-((S)-2,2-dimethylcyclopropane-1-carbonyl)-2,6-diazaspiro[3.4]octan-8-yl)phenyl)cyclohexanecarboxamide (60 mg, 41%) as a white solid. LCMS m/z=594.4 [M+H]⁺; ¹H NMR (400 MHz, Methanol-d₄) δ 8.30-8.24 (m, 1H), 7.97 (d, J=9.5 Hz, 1H), 7.65-7.55 (m, 2H), 7.38-7.24 (m, 7H), 5.38 (d, J=9.2 Hz, 2H), 4.34-4.05 (m, 4H), 4.04-3.84 (m, 4H), 3.60 (m, 1H), 2.41-2.29 (m, 1H), 1.90-1.79 (m, 4H), 1.72 (d, J=12.0 Hz, 1H), 1.58-1.46 (m, 2H), 1.43-1.13 (m, 6H), 1.10-1.06 (m, 1H), 1.05-1.02 (m, 1H), 0.97-0.82 (m, 2H), 0.76-0.60 (m, 2H).

### Synthesis of (8-(5-(1-(3-(2-oxabicyclo[2.2.2]octan-4-yl)-1H-pyrazol-1-yl)ethyl)-1,2,4-oxadiazol-3-yl)-2-(1-(trifluoromethyl)cyclopropane-1-carbonyl)-2,6-diazaspiro[3.4]octan-6-yl)(1-(4-(trifluoromethyl)benzyl)-1H-pyrazol-4-yl)methanone (I-217)

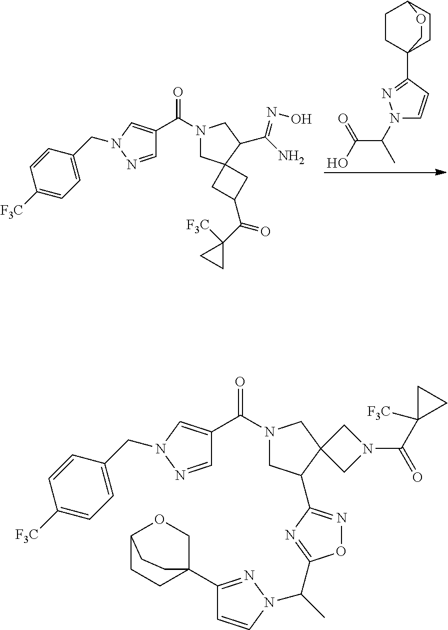 [SMILES: N/C(=N\O)C1CN(C(=O)c2cnn(Cc3ccc(C(F)(F)F)cc3)c2)CC12CC(C(=O)C1(C(F)(F)F)CC1)C2 ; CC(C(=O)O)n1ccc(C23CCC(CC2)OC3)n1 ; CC(c1nc(C2CN(C(=O)c3cnn(Cc4ccc(C(F)(F)F)cc4)c3)CC23CN(C(=O)C2(C(F)(F)F)CC2)C3)no1)n1ccc(C23CCC(CC2)OC3)n1]

To a solution of 2-(3-(2-oxabicyclo[2.2.2]octan-4-yl)-1H-pyrazol-1-yl)propanoic acid (25 mg, 0.1 mmol) in a mixture of DMF (0.5 mL) and dioxane (0.2 mL) in sealed tube was added (Z)—N′-hydroxy-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboximidamide (56 mg, 0.1 mmol) and EDCI (29 mg, 0.15 mmol) and the reaction heated at 60° C. for 3 h, the temperature was increased to 110° C. and stirring continued for 5 h. The mixture was purified by prep-HPLC to afford (8-(5-(1-(3-(2-oxabicyclo[2.2.2]octan-4-yl)-1H-pyrazol-1-yl)ethyl)-1,2,4-oxadiazol-3-yl)-2-(1-(trifluoromethyl)cyclopropane-1-carbonyl)-2,6-diazaspiro[3.4]octan-6-yl)(1-(4-(trifluoromethyl)benzyl)-1H-pyrazol-4-yl)methanone (16.5 mg, 43%) as a white solid. LCMS m/z=773.3 [M+H]⁺; ¹H NMR (400 MHz, DMSO-d₆) δ 8.42 (d, J=10.6 Hz, 1H), 7.89-7.78 (m, 2H), 7.72 (d, J=7.8 Hz, 2H), 7.44 (d, J=7.6 Hz, 2H), 6.18 (s, 1H), 6.04-5.84 (m, 1H), 5.48 (s, 2H), 4.53-4.24 (m, 1H), 4.11 (d, J=22.4 Hz, 3H), 4.02-3.76 (m, 5H), 3.73 (d, J=9.6 Hz, 3H), 1.97-1.75 (m, 9H), 1.62 (t, J=11.8 Hz, 2H), 1.17 (m, 4H).

### Synthesis of (8-(5-((3,4-dichlorobenzyl)((tetrahydro-2H-pyran-4-yl)methyl)amino)-1,2,4-oxadiazol-3-yl)-2-(1-(trifluoromethyl)cyclopropane-1-carbonyl)-2,6-diazaspiro[3.4]octan-6-yl)(1-(4-(trifluoromethyl)benzyl)-1H-pyrazol-4-yl)methanone (I-213)

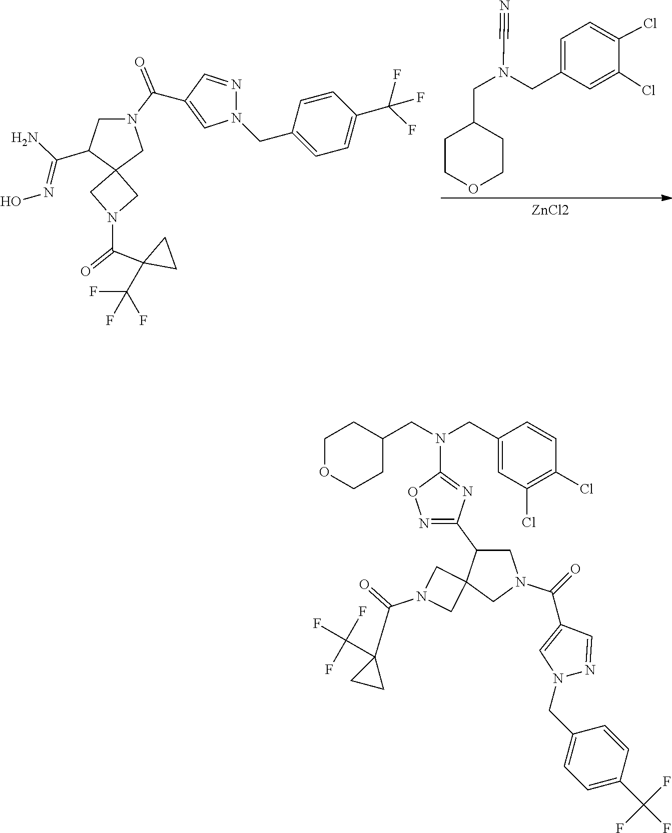

To a solution of N-(3,4-dichlorobenzyl)-N-((tetrahydro-2H-pyran-4-yl)methyl)cyanamide (240 mg, 0.83 mmol) in DMF (3 mL) was added TsOH (80 mg, 0.41 mmol), (Z)—N′-hydroxy-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboximidamide (466 mg, 0.83 mmol) and ZnCl₂ (1.0 M in solution in THF, 0.85 mL, 0.85 mmol). The reaction was heated at 50° C. under N₂ atmosphere for 3 h. The mixture was diluted with water (30 mL) and extracted with DCM (10 mL×3). The combine organic layers were washed with brine, dried over Na₂SO₄, filtered and concentrated. The mixture was purified by prep-HPLC to afford (8-(5-((3,4-dichlorobenzyl)((tetrahydro-2H-pyran-4-yl)methyl)amino)-1,2,4-oxadiazol-3-yl)-2-(1-(trifluoromethyl)cyclopropane-1-carbonyl)-2,6-diazaspiro[3.4]octan-6-yl)(1-(4-(trifluoromethyl)benzyl)-1H-pyrazol-4-yl)methanone (18.7 mg, 3%) as a white solid. LCMS m/z=840.0[M+H]⁺; ¹HNMR (400 MHz, DMSO-d6) δ 8.45-8.39 (m, 1H), 7.90-7.82 (m, 1H), 7.75-7.69 (m, 2H), 7.64-7.55 (m, 2H), 7.47-7.41 (m, 2H), 7.34-7.23 (m, 1H), 5.51-5.45 (m, 2H), 4.71-4.61 (m, 2H), 4.44-3.57 (m, 12H), 3.24-3.16 (m, 2H), 2.02-1.91 (m, 1H), 1.50-1.38 (m, 2H), 1.29-0.89 (m, 7H).

### Synthesis of (8-(5-(2-(benzyloxy)-1-(3-cyclohexylphenyl)ethyl)-1,3,4-oxadiazol-2-yl)-2-(1-(trifluoromethyl)cyclopropane-1-carbonyl)-2,6-diazaspiro[3.4]octan-6-yl)(5-hydroxypyrazin-2-yl)methanone (I-107)

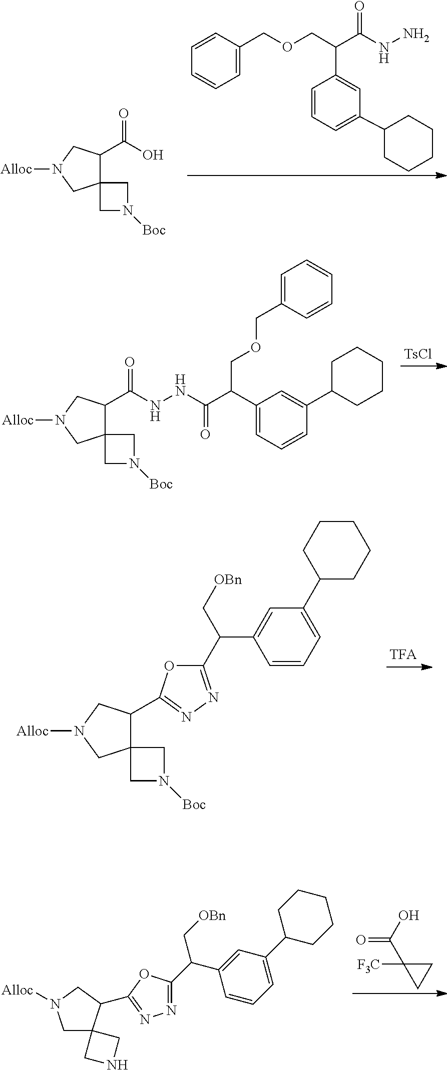

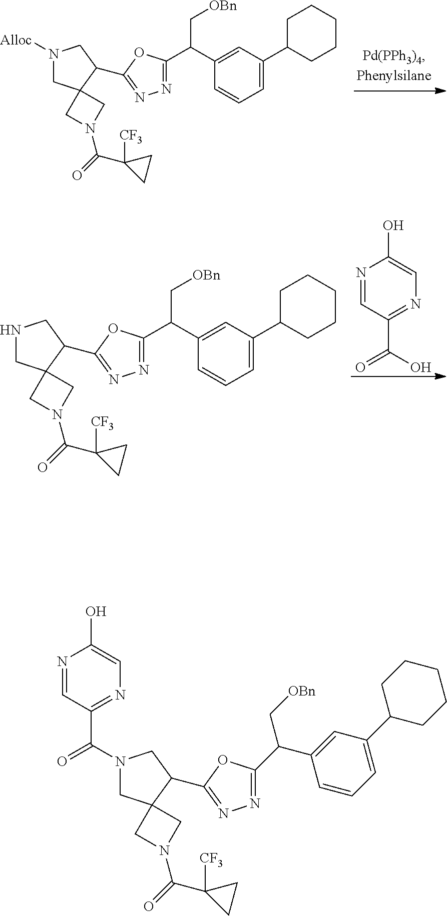

### Step 1: 6-allyl 2-(tert-butyl) 8-(2-(3-(benzyloxy)-2-(3-cyclohexylphenyl)propanoyl)hydrazine-1-carbonyl)-2,6-diazaspiro[3.4]octane-2,6-dicarboxylate

To a solution of 6-((allyloxy)carbonyl)-2-(tert-butoxycarbonyl)-2,6-diazaspiro[3.4]octane-8-carboxylic acid (488 mg, 1.4 mmol) in DCM (8 mL) was added HATU (596 mg, 1.5 mmol) and DIEA (505 mg, 3.9 mmol) and the mixture stirred for 30 min. 3-(benzyloxy)-2-(3-cyclohexylphenyl)propanehydrazide (460 mg, 1.3 mmol) was added and stirring continued for 3 h. The mixture was diluted with water (20 mL) and extracted with DCM (50 mL×2). The combined organic layers were washed with brine, dried over Na₂SO₄, filtered and concentrated. The residue was purified by RP-column to afford 6-allyl 2-(tert-butyl) 8-(2-(3-(benzyloxy)-2-(3-cyclohexylphenyl)propanoyl)hydrazine-1-carbonyl)-2,6-diazaspiro[3.4]octane-2,6-dicarboxylate (500 mg, 57%) as a white solid. LCMS m/z=LCMS m/z=675.1 [M+H]⁺; ¹H NMR (400 MHz, CD₃OD) δ 7.52-7.28 (m, 9H), 6.15 (s, 1H), 5.57-5.35 (m, 2H), 5.06 (s, 4H), 4.44-4.21 (m, 3H), 4.18-3.74 (m, 9H), 2.74-2.63 (m, 1H), 2.24-2.20 (m, 2H), 2.10-1.91 (m, 5H), 1.70-1.56 (m, 9H), 1.55-1.31 (m, 3H).

### Step 2: 6-allyl 2-(tert-butyl) 8-(5-(2-(benzyloxy)-1-(3-cyclohexylphenyl)ethyl)-1,3,4-oxadiazol-2-yl)-2,6-diazaspiro[3.4]octane-2,6-dicarboxylate

To a solution of 6-allyl 2-(tert-butyl) 8-(2-(3-(benzyloxy)-2-(3-cyclohexylphenyl)propanoyl)hydrazine-1-carbonyl)-2,6-diazaspiro[3.4]octane-2,6-dicarboxylate (500 mg, 0.74 mmol) in DCM (5 mL) was added TEA (374 mg, 3.7 mmol) and TsCl (704 mg, 3.7 mmol) and the reaction stirred for 2 h. The mixture was diluted with water (30 mL), extracted with DCM (50 mL×3). The combined organic layers were washed with brine, dried over Na₂SO₄, filtered and concentrated. The residue obtained was purified by column chromatography on silica gel (eluent: DCM:MeOH=40:1) to afford 6-allyl 2-(tert-butyl) 8-(5-(2-(benzyloxy)-1-(3-cyclohexylphenyl)ethyl)-1,3,4-oxadiazol-2-yl)-2,6-diazaspiro[3.4]octane-2,6-dicarboxylate (420 mg, 86%) as a yellow oil. ¹H NMR (400 MHz, CD₃OD) δ 7.35-7.06 (m, 9H), 5.94 (s, 1H), 5.35-5.15 (m, 2H), 4.65-4.53 (m, 4H), 4.19-3.66 (m, 12H), 1.90-1.71 (m, 6H), 1.50-1.36 (m, 14H).

### Step 3: allyl 8-(5-(2-(benzyloxy)-1-(3-cyclohexylphenyl)ethyl)-1,3,4-oxadiazol-2-yl)-2,6-diazaspiro[3.4]octane-6-carboxylate

To a solution of 6-allyl 2-(tert-butyl) 8-(5-(2-(benzyloxy)-1-(3-cyclohexylphenyl)ethyl)-1,3,4-oxadiazol-2-yl)-2,6-diazaspiro[3.4]octane-2,6-dicarboxylate (420 mg, 0.64 mmol) in DCM (6 mL) was added TFA (2 mL) and the reaction stirred at room temperature for 1 h. The solvent was removed under vacuum to afford allyl 8-(5-(2-(benzyloxy)-1-(3-cyclohexylphenyl)ethyl)-1,3,4-oxadiazol-2-yl)-2,6-diazaspiro[3.4]octane-6-carboxylate (355 mg, 100%) as a oil. LCMS m/z=557.1 [M+H]⁺.

### Step 4: allyl 8-(5-(2-(benzyloxy)-1-(3-cyclohexylphenyl)ethyl)-1,3,4-oxadiazol-2-yl)-2-(1-(trifluoromethyl)cyclopropane-1-carbonyl)-2,6-diazaspiro[3.4]octane-6-carboxylate

To a solution of 1-(trifluoromethyl)cyclopropane-1-carboxylic acid (108 mg, 0.7 mmol) in DCM (4 mL) was added HATU (291 mg, 0.77 mmol) and DIEA (247 mg, 1.9 mmol) and the mixture stirred for 30 min. Allyl 8-(5-(2-(benzyloxy)-1-(3-cyclohexylphenyl)ethyl)-1,3,4-oxadiazol-2-yl)-2,6-diazaspiro[3.4]octane-6-carboxylate (355 mg, 0.64 mmol) was added and stirring continued for 3 h. The mixture was diluted with water (20 mL) and extracted with DCM (50 mL×2). The combined organic layers were washed with brine, dried over Na₂SO₄, filtered and concentrated. The residue was purified by RP-column to afford allyl 8-(5-(2-(benzyloxy)-1-(3-cyclohexylphenyl)ethyl)-1,3,4-oxadiazol-2-yl)-2-(1-(trifluoromethyl)cyclopropane-1-carbonyl)-2,6-diazaspiro[3.4]octane-6-carboxylate (180 mg, 41%) as a yellow solid. ¹H NMR (400 MHz, CD₃OD) δ 7.32-7.06 (m, 9H), 6.02-5.88 (m, 1H), 5.36-5.17 (m, 2H), 4.65-4.52 (m, 4H), 4.15 (t, J=9.2 Hz, 2H), 3.98-3.67 (m, 10H), 2.50 (s, 1H), 1.90-1.72 (m, 9H), 1.37-1.22 (m, 2H), 1.14 (s, 3H)

### Step 5: (8-(5-(2-(benzyloxy)-1-(3-cyclohexylphenyl)ethyl)-1,3,4-oxadiazol-2-yl)-2,6-diazaspiro[3.4]octan-2-yl)(1-(trifluoromethyl)cyclopropyl)methanone

To a solution of allyl 8-(5-(2-(benzyloxy)-1-(3-cyclohexylphenyl)ethyl)-1,3,4-oxadiazol-2-yl)-2-(1-(trifluoromethyl)cyclopropane-1-carbonyl)-2,6-diazaspiro[3.4]octane-6-carboxylate (100 mg, 0.14 mmol) in DCM (1 mL) was added Pd(PPh₃)₄ (16 mg, 0.01 mmol) and phenylsilane (78 mg, 0.72 mmol). The reaction was stirred at room temperature for 2 h, the solvent removed and the residue obtained purified by RP-column to afford (8-(5-(2-(benzyloxy)-1-(3-cyclohexylphenyl)ethyl)-1,3,4-oxadiazol-2-yl)-2,6-diazaspiro[3.4]octan-2-yl)(1-(trifluoromethyl)cyclopropyl)methanone (50 mg, 57%). LCMS m/z=608.9[M+H]⁺.

### Step 6: (8-(5-(2-(benzyloxy)-1-(3-cyclohexylphenyl)ethyl)-1,3,4-oxadiazol-2-yl)-2-(1-(trifluoromethyl)cyclopropane-1-carbonyl)-2,6-diazaspiro[3.4]octan-6-yl)(5-hydroxypyrazin-2-yl)methanone (I-107)

To a solution of 5-hydroxypyrazine-2-carboxylic acid (14 mg, 0.1 mmol) in DCM (4 mL) was added HATU (38 mg, 0.1 mmol) and DIEA (31 mg, 0.25 mmol) and the mixture stirred for 30 min. (8-(5-(2-(benzyloxy)-1-(3-cyclohexylphenyl)ethyl)-1,3,4-oxadiazol-2-yl)-2,6-diazaspiro[3.4]octan-2-yl)(1-(trifluoromethyl)cyclopropyl)methanone (50 mg, 0.08 mmol) was added and stirring continued for 3 h. The mixture was diluted with water (20 mL) and extracted with DCM (50 mL×2). The combined organic layers were washed with brine, dried over Na₂SO₄, filtered and concentrated. The residue was purified by RP-column to afford (8-(5-(2-(benzyloxy)-1-(3-cyclohexylphenyl)ethyl)-1,3,4-oxadiazol-2-yl)-2-(1-(trifluoromethyl)cyclopropane-1-carbonyl)-2,6-diazaspiro[3.4]octan-6-yl)(5-hydroxypyrazin-2-yl)methanone (30 mg, 48%) as a white solid. LCMS m/z=731.2 [M+H]⁺; ¹H NMR (400 MHz, CD3OD) δ 8.05-7.94 (m, 2H), 7.33-7.03 (m, 9H), 4.61 (p, J=6.9, 6.4 Hz, 1H), 4.55-4.51 (m, 2H), 4.39-4.31 (m, 2H), 4.20-3.88 (m, 7H), 3.68-3.46 (m, 1H), 2.57-2.38 (m, 1H), 1.89-1.70 (m, 5H), 1.49-1.33 (m, 5H), 1.20-0.89 (m, 4H).

### Synthesis of (1-((1H-benzo[d]imidazol-4-yl)methyl)-1H-pyrazol-4-yl)(8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octan-6-yl)methanone (I-108)

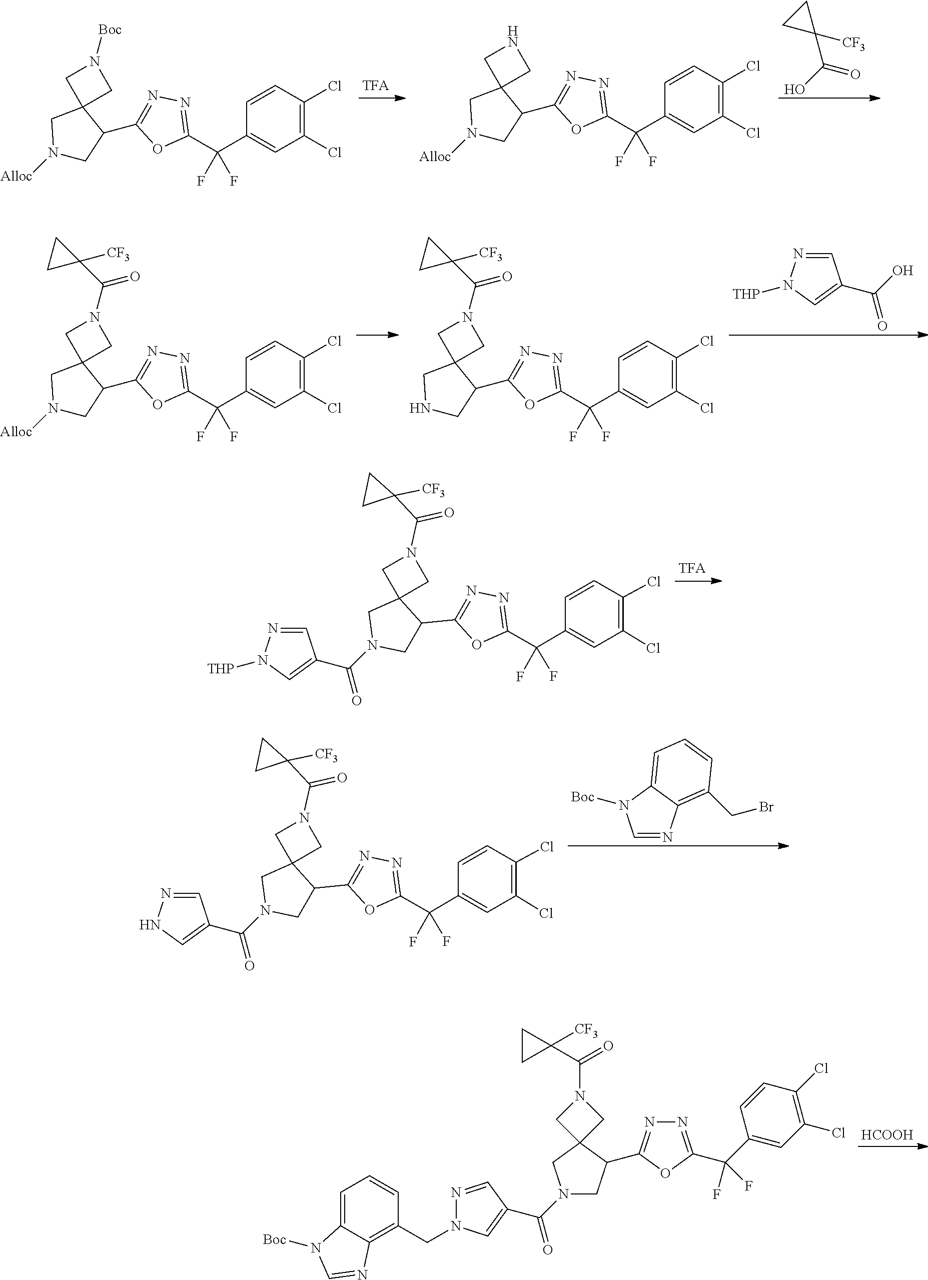

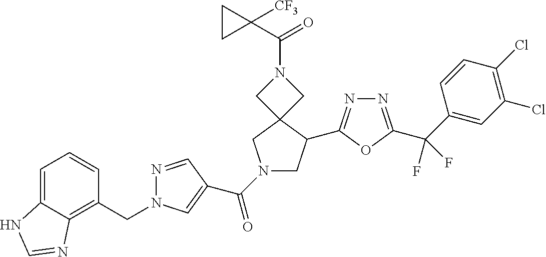 [SMILES: O=C(c1cnn(Cc2cccc3[nH]cnc23)c1)N1CC(c2nnc(C(F)(F)c3ccc(Cl)c(Cl)c3)o2)C2(C1)CN(C(=O)C1(C(F)(F)F)CC1)C2]

### Step 1: allyl 8-(5-((3,4-dichlorophenyl)difluoromethyl)-1,3,4-oxadiazol-2-yl)-2,6-diazaspiro[3.4]octane-6-carboxylate

To a solution of 6-allyl 2-(tert-butyl) 8-(5-((3,4-dichlorophenyl)difluoromethyl)-1,3,4-oxadiazol-2-yl)-2,6-diazaspiro[3.4]octane-2,6-dicarboxylate (500 mg, 0.89 mmol) in DCM (1.0 mL) was added TFA (5.0 mL) and the reaction stirred at room temperature for 2 h. The solvent was removed under vacuum to afford allyl 8-(5-((3,4-dichlorophenyl)difluoromethyl)-1,3,4-oxadiazol-2-yl)-2,6-diazaspiro[3.4]octane-6-carboxylate (410 mg, quant.) which was used directly for the next step. LCMS m/z=459.1[M+H]⁺.

### Step 2: allyl 8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octane-6-carboxylate

To a solution of solution of 1-(trifluoromethyl)cyclopropane-1-carboxylic acid (140 mg, 0.96 mmol) in DMF (5.0 mL) was added HATU (364 mg, 0.96 mmol) and DIPEA (337 mg, 2.61 mmol) and the mixture stirred for 30 min. Allyl 8-(5-((3,4-dichlorophenyl)difluoromethyl)-1,3,4-oxadiazol-2-yl)-2,6-diazaspiro[3.4]octane-6-carboxylate (400 mg, 0.87 mmol) was added and stirring continued for 2 h. The mixture was diluted with water (50 mL) and extracted with EtOAc (80 mL×2). The combined organic layers were washed with brine, dried over Na₂SO₄, filtered and concentrated. The crude product was purified by column chromatography on silica gel (DCM:MeOH=75:1) to afford allyl 8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octane-6-carboxylate (420 mg, 81%) as a yellow oil. LCMS m/z=595.1 [M+H]⁺; ¹H NMR (400 MHz, CDCl₃) δ 7.76 (d, J=2.1 Hz, 1H), 7.64-7.58 (m, 1H), 7.54-7.46 (m, 1H), 6.02-5.83 (m, 1H), 5.35-5.21 (m, 2H), 4.68-4.57 (m, 2H), 4.46-4.00 (m, 4H), 3.96-3.86 (m, 3H), 3.82-3.70 (m, 2H), 1.23-1.16 (m, 4H).

### Step 3: (8-(5-((3,4-dichlorophenyl)difluoromethyl)-1,3,4-oxadiazol-2-yl)-2,6-diazaspiro[3.4]octan-2-yl)(1-(trifluoromethyl)cyclopropyl)methanone

To a solution of allyl 8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octane-6-carboxylate (1.36 g, 2.28 mmol) in THF (15 mL) was added Phenylsilane (1.24 g, 11.42 mmol) and Pd(PPh₃)₄ (264 mg, 0.23 mmol). The reaction mixture was stirred at room temperature for 20 min then was diluted with water (20 mL) and extracted with EtOAc (40 mL×2). The combined organic layers were washed with brine, dried over Na₂SO₄, filtered and concentrated. The residue was purified by column chromatography on silica gel (DCM:MeOH=30:1) to afford (8-(5-((3,4-dichlorophenyl)difluoromethyl)-1,3,4-oxadiazol-2-yl)-2,6-diazaspiro[3.4]octan-2-yl)(1-(trifluoromethyl)cyclopropyl)methanone (700 mg, 60%) as a brown oil. LCMS m/z=510.8 [M+H]⁺; ¹H NMR (400 MHz, CD₃OD) δ 7.89 (d, J=2.1 Hz, 1H), 7.75 (d, J=8.4 Hz, 1H), 7.65-7.59 (m, 1H), 3.69-3.39 (m, 9H), 1.20 (s, 4H).

### Step 4: (8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octan-6-yl)(1-(tetrahydro-2H-pyran-2-yl)-1H-pyrazol-4-yl)methanone

To a solution of solution of 1-(tetrahydro-2H-pyran-2-yl)-1H-pyrazole-4-carboxylic acid (58 mg, 0.29 mmol) in DMF (1.0 mL) was added HATU (112 mg, 0.29 mmol) and DIPEA (101 mg, 0.78 mmol) and the mixture stirred for 30 min. (8-(5-((3,4-dichlorophenyl)difluoromethyl)-1,3,4-oxadiazol-2-yl)-2,6-diazaspiro[3.4]octan-2-yl)(1-(trifluoromethyl)cyclopropyl)methanone (100 mg, 0.20 mmol) was added and stirring continued for 2 h. The mixture was purified by RP-column (water:CH₃CN=30:70) to afford (8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octan-6-yl)(1-(tetrahydro-2H-pyran-2-yl)-1H-pyrazol-4-yl)methanone (72 mg, 51%) as a colorless oil. LCMS m/z=690.8 [M+H]⁺; ¹H NMR (400 MHz, CD₃OD) δ 8.27 (d, J=6.2 Hz, 1H), 7.93 (d, J=6.6 Hz, 1H), 7.91-7.85 (m, 1H), 7.74 (d, J=8.5 Hz, 1H), 7.65-7.58 (m, 1H), 5.46 (dd, J=9.7, 2.3 Hz, 1H), 4.55-4.01 (m, 10H), 3.81-3.70 (m, 1H), 2.10-1.61 (m, 6H), 1.21 (s, 4H).

### Step 5: (8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octan-6-yl)(1H-pyrazol-4-yl)methanone

To a solution of 8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octan-6-yl)(1-(tetrahydro-2H-pyran-2-yl)-1H-pyrazol-4-yl)methanone (629 mg, 0.91 mmol) in DCM (10.0 mL) was added TFA (5.0 mL) and the reaction stirred at room temperature for 2 h. The solvent was removed under reduced pressure and the residue obtained diluted with water (40 mL) and made basic with aq NaHCO₃ to pH˜9. The aqueous was extracted with EtOAc (50 mL×3) and the combined organic layers washed with brine, dried over Na₂SO₄, filtered and concentrated to afford (8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octan-6-yl)(1H-pyrazol-4-yl)methanone (550 mg, quant.) as a yellow oil which was used directly in the next step. LCMS m/z=604.9 [M+H]⁺.

### Step 6: tert-butyl 4-((4-(8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octane-6-carbonyl)-1H-pyrazol-1-yl)methyl)-1H-benzo[d]imidazole-1-carboxylate

To a solution of (8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octan-6-yl)(1H-pyrazol-4-yl)methanone (50 mg, 0.082 mmol) in acetonitrile (1.0 mL) was added cesium carbonate (81 mg, 0.25 mmol) and tert-butyl 4-(bromomethyl)-1H-benzo[d]imidazole-1-carboxylate (27 mg, 0.082 mmol). The reaction was stirred at room temperature for 3 h then was filtered through Celite and the filtrate concentrated. The residue obtained was purified by prep-TLC (DCM:MeOH=15:1) to afford tert-butyl 4-((4-(8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octane-6-carbonyl)-1H-pyrazol-1-yl)methyl)-1H-benzo[d]imidazole-1-carboxylate (50 mg, 72%) as a colorless solid. LCMS m/z=834.9 [M+H]⁺; ¹H NMR (400 MHz, CD₃OD) δ 8.61 (s, 1H), 8.33 (s, 1H), 8.02 (d, J=8.2 Hz, 1H), 7.94-7.85 (m, 2H), 7.72 (d, J=8.5 Hz, 1H), 7.64-7.58 (m, 1H), 7.41 (t, J=7.9 Hz, 1H), 7.24 (s, 1H), 5.78 (s, 2H), 4.29-3.97 (m, 9H), 1.72-1.69 (m, 9H), 1.21 (s, 4H).

### Step 7: (1-((1H-benzo[d]imidazol-4-yl)methyl)-1H-pyrazol-4-yl)(8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octan-6-yl)methanone (I-108)

A solution of tert-butyl 4-((4-(8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octane-6-carbonyl)-1H-pyrazol-1-yl)methyl)-1H-benzo[d]imidazole-1-carboxylate (40 mg, 0.048 mmol) in formic acid (2.0 mL) was stirred at room temperature for 2 h. The solvent was removed under vacuum and the residue obtained purified by RP-column (water:CH₃CN=30:70) to afford (1-((1H-benzo[d]imidazol-4-yl)methyl)-1H-pyrazol-4-yl)(8-(5-((3,4-dichlorophenyl)difluoromethyl)-1,3,4-oxadiazol-2-yl)-2-(1-(trifluoromethyl)cyclopropane-1-carbonyl)-2,6-diazaspiro[3.4]octan-6-yl)methanone (16 mg, 46%) as a white solid. LCMS m/z=735.2 [M+H]⁺; ¹H NMR (400 MHz, CD₃OD) δ 8.27 (d, J=10.9 Hz, 1H), 8.23 (s, 1H), 7.91 (d, J=6.1 Hz, 1H), 7.87 (d, J=2.6 Hz, 1H), 7.72 (d, J=8.4 Hz, 1H), 7.63-7.55 (m, 2H), 7.25 (t, J=7.8 Hz, 1H), 7.11 (d, J=7.4 Hz, 1H), 5.75 (s, 2H), 4.35-3.88 (m, 9H), 1.20 (s, 4H).

Table 35: The compounds listed in Table 35 were synthesized from according to the procedures outlined for I-108 using the appropriate commercially available reagents and/or intermediates described elsewhere.

| Example | | | |
|---|---|---|---|
| Number | Compound | ¹HNMR | LCMS |
| I-106 | 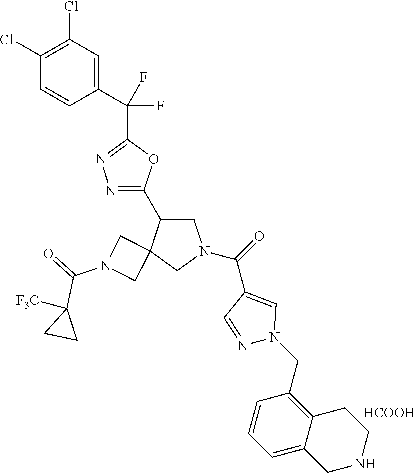 [SMILES: O=C(c1cnn(Cc2cccc3c2CCNC3)c1)N1CC(c2nnc(C(F)(F)c3ccc(Cl)c(Cl)c3)o2)C2(C1)CN(C(=O)C1(C(F)(F)F)CC1)C2 ; "HCOOH"] | ¹H NMR (400 MHz, Methanol-d₄) δ 8.50 (s, 1H), 8.14 (d, J = 8.4 Hz, 1H), 7.93 (d, J = 6.5 Hz, 1H), 7.91 − 7.85 (m, 1H), 7.75 (d, J = 8.5 Hz, 1H), 7.65 − 7.58 (m, 1H), 7.28 (t, J = 7.7 Hz, 1H), 7.20 (d, J = 7.7 Hz, 1H), 7.12 (d, J = 7.6 Hz, 1H), 5.43 (s, 2H), 4.42 − 3.86 (m, 10H), 3.46 (t, J = 6.4 Hz, 2H), 3.05 (t, J = 6.5 Hz, 2H), 1.27 − 1.16 (m, 4H). | m/z 750.1 [M + H]⁺ |

### Synthesis of 2-(4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)acetic acid (I-110)

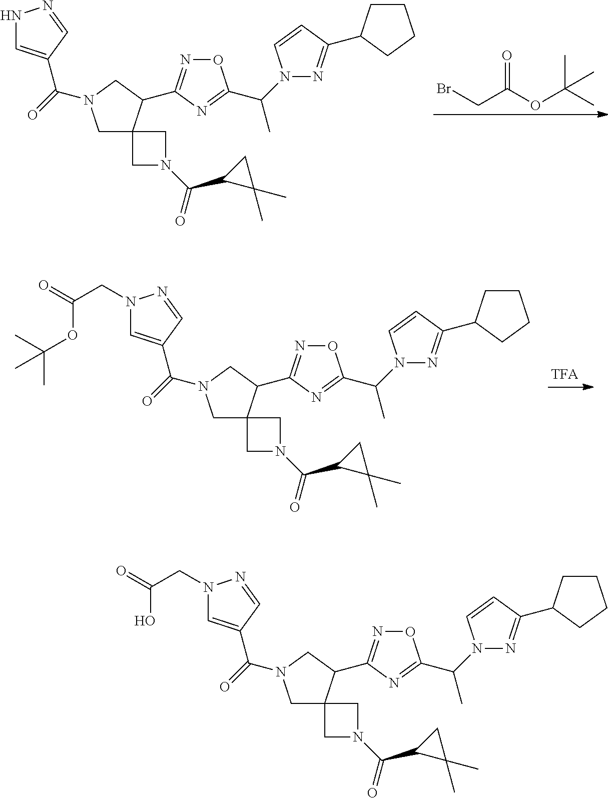

### Step 1: tert-butyl 2-(4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)acetate

To a solution of (8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(1H-pyrazol-4-yl)methanone (30 mg, 0.06 mmol) in DMF (1 mL) was added tert-butyl 2-bromoacetate (35 mg, 0.18 mmol) and Cs₂CO₃ (98 mg, 0.3 mmol). The reaction was heated at 50° C. overnight then the solvent was removed. The residue obtained was purified by RP-column to afford tert-butyl 2-(4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)acetate (40 mg, 70%) as a colorless oil. ¹H NMR (400 MHz, Chloroform-d) δ 7.92 (s, 1H), 7.85 (s, 1H), 7.46 (s, 1H), 6.14 (s, 1H), 5.74 (s, 1H), 4.83 (s, 2H), 4.26-3.62 (m, 9H), 3.07 (t, J=8.1 Hz, 1H), 2.08-1.93 (m, 5H), 1.76-1.61 (m, 6H), 1.48 (s, 9H), 1.16-1.03 (m, 8H), 0.78-0.68 (m, 1H).

### Step 2: 2-(4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)acetic acid

To a solution of tert-butyl 2-(4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)acetate (40 mg, 0.06 mmol) in DCM (1 mL) was added TFA (0.5 mL) and the reaction stirred at room temperature for 4 h. The mixture was purified by RP-column to afford 2-(4-(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octane-6-carbonyl)-1H-pyrazol-1-yl)acetic acid (13 mg, 36%) as a white solid. LCMS: m/z=591.2 [M+H]⁺; ¹H NMR (400 MHz, CD₃OD) δ 8.22 (d, J=10.2 Hz, 1H), 7.97-7.89 (m, 1H), 7.70 (s, 1H), 6.18 (s, 1H), 5.88 (s, 1H), 5.04 (d, J=4.2 Hz, 2H), 4.40-3.85 (m, 9H), 3.08-2.98 (m, 1H), 1.94 (d, J=6.8 Hz, 5H), 1.76 (s, 2H), 1.68-1.55 (m, 4H), 1.48-1.26 (m, 2H), 1.20-1.01 (m, 7H), 0.76 (s, 1H).

### Synthesis of (8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(1-((1-methyl-1H-tetrazol-5-yl)methyl)-1H-pyrazol-4-yl)methanone (I-1H)

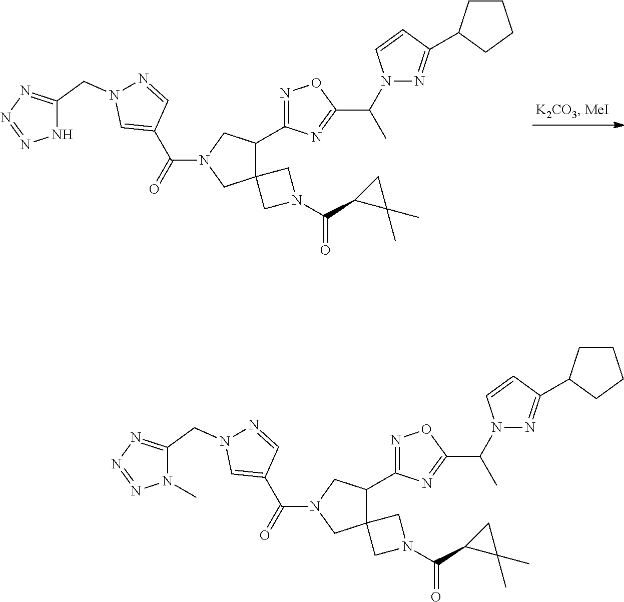 [SMILES: CC(c1nc(C2CN(C(=O)c3cnn(Cc4nnn[nH]4)c3)CC23CN(C(=O)[C@H]2CC2(C)C)C3)no1)n1ccc(C2CCCC2)n1 ; CC(c1nc(C2CN(C(=O)c3cnn(Cc4nnnn4C)c3)CC23CN(C(=O)[C@H]2CC2(C)C)C3)no1)n1ccc(C2CCCC2)n1]

### Step 1: (8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(1-((1-methyl-1H-tetrazol-5-yl)methyl)-1H-pyrazol-4-yl)methanone

To a solution of (1-((1H-tetrazol-5-yl)methyl)-1H-pyrazol-4-yl)(8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)methanone (20 mg, 32.5 umol) in DMF (0.5 ml) was added K₂CO₃ (14 mg, 97.60 umol) and Mel (14 mg, 97.60 umol) and the reaction stirred overnight at room temperature. The mixture was purified by prep-HPLC to afford (8-(5-(1-(3-cyclopentyl-1H-pyrazol-1-yl)ethyl)-1,2,4-oxadiazol-3-yl)-2-((S)-2,2-dimethylcyclopropane-1-carbonyl)-2,6-diazaspiro[3.4]octan-6-yl)(1-((1-methyl-1H-tetrazol-5-yl)methyl)-1H-pyrazol-4-yl)methanone (11 mg, 55%) as a white solid. LCMS m/z=629.3 [M+H]⁺; ¹H NMR (400 MHz, CD₃OD) δ 8.40-8.28 (m, 1H), 7.92 (t, J=9.1 Hz, 1H), 7.73-7.63 (m, 1H), 6.17 (s, 1H), 5.94-5.78 (m, 2H), 5.70-5.67 (m, 1H), 4.41-3.80 (m, 12H), 3.10-2.96 (m, 1H), 2.04-1.91 (m, 5H), 1.81-1.55 (m, 6H), 1.46-1.28 (m, 1H), 1.20-1.00 (m, 7H), 0.81-0.71 (m, 1H).

### Synthesis of methyl 5-(trifluoromethyl)-2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoate (I-219)

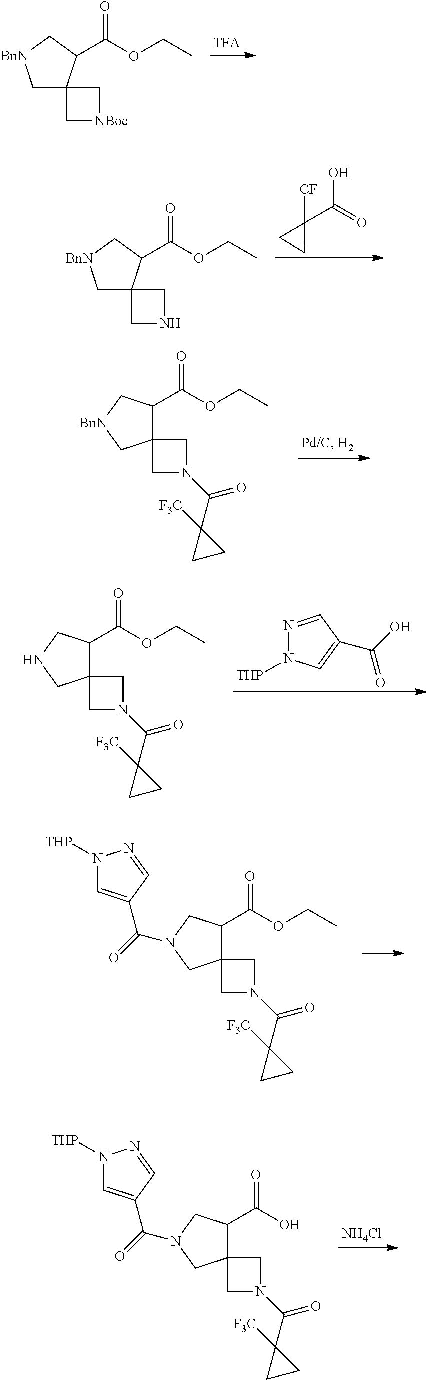

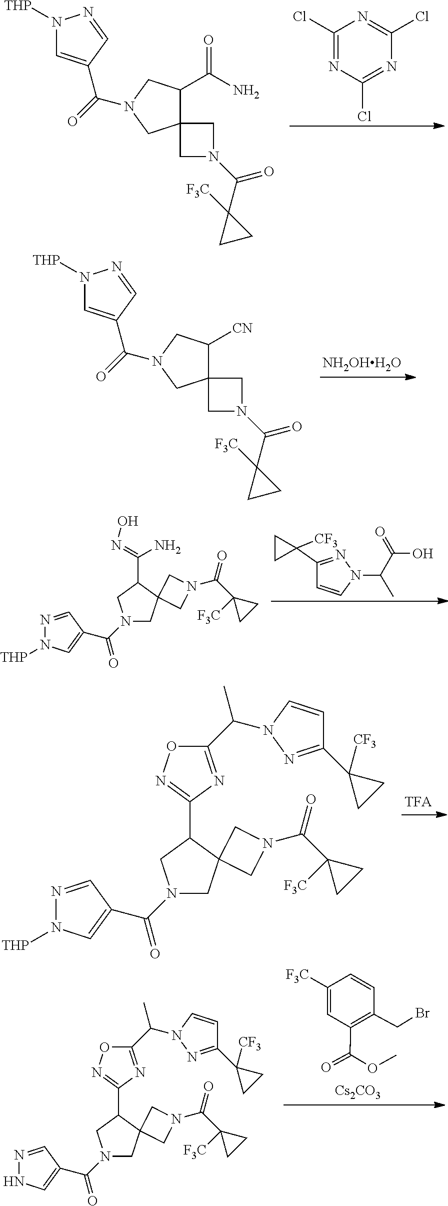

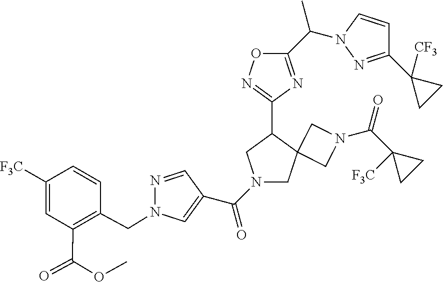 [SMILES: COC(=O)c1cc(C(F)(F)F)ccc1Cn1cc(C(=O)N2CC(c3noc(C(C)n4ccc(C5(C(F)(F)F)CC5)n4)n3)C3(C2)CN(C(=O)C2(C(F)(F)F)CC2)C3)cn1]

### Step 1: ethyl 6-benzyl-2,6-diazaspiro[3.4]octane-8-carboxylate

To a solution of 2-(tert-butyl) 8-ethyl 6-benzyl-2,6-diazaspiro[3.4]octane-2,8-dicarboxylate (10.0 g, 26.7 mmol) in DCM (100 mL) was added TFA (30.0 mL) and the reaction stirred at room temperature for 2. The solvent was removed under vacuum to afford ethyl 6-benzyl-2,6-diazaspiro[3.4]octane-8-carboxylate (7.33 g, quant.) which was used directly in the next step. LCMS m/z=274.7 [M+H]⁺; ¹HNMR (400 MHz, CD₃OD) δ 7.31-7.11 (m, 5H), 4.20-4.09 (m, 2H), 4.09-3.96 (m, 4H), 3.95-3.82 (m, 2H), 3.69-3.34 (m, 4H), 3.40-3.36 (m, 1H), 1.08-0.95 (m, 3H).

### Step 2: ethyl 6-benzyl-2-(1-(trifluoromethyl)cyclopropane-1-carbonyl)-2,6-diazaspiro[3.4]octane-8-carboxylate

To a solution of solution of 1-(trifluoromethyl)cyclopropane-1-carboxylic acid (4.53 g, 29.4 mmol) in DMF (70 mL) was added HATU (11.17 g, 29.39 mmol) and DIPEA (19.0 mL, 106.88 mmol) and the mixture was stirred at room temperature for 30 min. Ethyl 6-benzyl-2,6-diazaspiro[3.4]octane-8-carboxylate e (7.33 g, 26.72 mmol) was added and the reaction was stirred for another 2 h. The solvent was removed under vacuum and the residue obtained purified by RP-column (20% water in CH₃CN) to afford ethyl 6-benzyl-2-(1-(trifluoromethyl)cyclopropane-1-carbonyl)-2,6-diazaspiro[3.4]octane-8-carboxylate (10.8 g, 85%) as a yellow oil. LCMS m/z=410.9 [M+H]⁺; ¹H NMR (400 MHz, DMSO-d₆) δ 7.60-7.41 (m, 5H), 4.48-3.39 (m, 13H), 1.20 (dd, J=15.5, 7.4 Hz, 7H).

### Step 3: ethyl 2-(1-(trifluoromethyl)cyclopropane-1-carbonyl)-2,6-diazaspiro[3.4]octane-8-carboxylate

To a solution of ethyl 6-benzyl-2-(1-(trifluoromethyl)cyclopropane-1-carbonyl)-2,6-diazaspiro[3.4]octane-8-carboxylate (2.2 g, 5.35 mmol) in methanol (3.0 mL) was added 10% Pd/C (500 mg) and the reaction stirred under a H₂ atmosphere overnight. The catalyst was removed by filtration through Celite and the filtrate concentrated to afford ethyl 2-(1-(trifluoromethyl)cyclopropane-1-carbonyl)-2,6-diazaspiro[3.4]octane-8-carboxylate (1.7 g, quant.) as a yellow oil which was used without further purification. LCMS m/z=321.3 [M+H]⁺.

### Step 4: ethyl 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboxylate

To a solution of 1-(tetrahydro-2H-pyran-2-yl)-1H-pyrazole-4-carboxylic acid (1.31 g, 6.70 mmol) in DMF (20 mL) was added HATU (2.55 g, 6.70 mmol) and DIPEA (3.24 mL, 18.26 mmol) and the mixture stirred at room temperature for 30 min. Ethyl 2-(1-(trifluoromethyl)cyclopropane-1-carbonyl)-2,6-diazaspiro[3.4]octane-8-carboxylate (1.95 g, 6.09 mmol) was added and stirring continued for 2 h. The mixture was diluted with water (50 mL) and extracted with EtOAc (100 mL×2). The combined organic layers were washed with brine, dried over Na₂SO₄, filtered and concentrated. The residue obtained was purified by column chromatography on silica gel (eluent: DCM:methanol=40:1) to afford ethyl 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboxylate (2.0 g, 85%) as a white solid. LCMS m/z=499.1 [M+H]⁺; ¹H NMR (400 MHz, CD₃OD) δ 8.26 (d, J=6.2 Hz, 1H), 7.92 (d, J=6.4 Hz, 1H), 5.48-5.43 (m, 1H), 4.28-3.65 (m, 11H), 3.56-3.37 (m, 2H), 2.07-1.99 (m, 2H), 1.83-1.49 (m, 4H), 1.31-1.20 (m, 7H).

### Step 5: 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboxylic acid

To a solution of ethyl 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboxylate (1.90 g, 3.81 mmol) in a mixture of THF and H₂O (20 mL/4 mL) was added LiOH·H₂O (400 mg, 9.53 mmol) and the reaction stirred at room temperature for 2 h. The reaction was diluted with water (30 mL) and extracted with EtOAc (30 mL). The aqueous layer was collected, acidified with 1M HCl to pH˜2 and extracted with EtOAc (50 mL×3). The combined organic layers were washed with brine, dried over Na₂SO₄, filtered and concentrated to afford 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboxylic acid (1.4 g, 80%) as a white solid. LCMS m/z=471.2 [M+H]⁺; ¹H NMR (400 MHz, CD₃OD) δ 8.05 (d, J=6.3 Hz, 1H), 7.71 (d, J=5.8 Hz, 1H), 5.30-5.22 (m, 1H), 4.24-3.24 (m, 11H), 2.01-1.82 (m, 2H), 1.64-1.27 (m, 4H), 1.05-1.00 (m, 4H).

### Step 6: 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboxamide

To a solution of 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboxylic acid (4.3 g, 9.14 mmol) in DMF (4.5 mL) was added HATU (5.21 g, 13.71 mmol) and DIPEA (3.54 mg, 27.42 mmol) and the mixture stirred at room temperature for 30 min. NH₄Cl (1.47 g, 27.42 mmol) was added and stirring continued overnight. The solvent was removed under reduced pressure and the residue was purified by RP-column (30% water in CH₃CN) to afford 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboxamide (4.1 g, 96%) as a white solid. LCMS m/z=470.3 [M+H]⁺; ¹H NMR (400 MHz, CD₃OD) δ 8.26 (d, J=9.3 Hz, 1H), 7.92 (d, J=7.5 Hz, 1H), 5.45 (d, J=9.5 Hz, 1H), 4.36-3.60 (m, 11H), 2.06-2.02 (m, 2H), 1.75-1.61 (m, 4H), 1.25-1.21 (m, 4H).

### Step 7: 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carbonitrile

To a solution of 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboxamide (4.1 g, 8.73 mmol) in DMF (4.0 mL) at 0° C. was added 2,4,6-trichloro-1,3,5-triazine (1.93 g, 10.5 mmol) and the reaction stirred for 2 h. The mixture was diluted with water (50 mL) and extracted with EtOAc (80 mL×2). The combined organic layers were washed with brine, dried over Na₂SO₄, filtered and concentrated. The residue obtained was purified by column chromatography on silica gel (eluent: DCM:methanol=50:1) to afford 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carbonitrile (1.9 g, 48%) as a white solid. LCMS m/z=452.3 [M+H]⁺; ¹H NMR (400 MHz, CD₃OD) δ 8.28 (d, J=7.4 Hz, 1H), 7.93 (d, J=10.2 Hz, 1H), 5.48-5.43 (m, 1H), 4.34-3.46 (m, 11H), 2.03 (d, J=11.6 Hz, 2H), 1.88-1.50 (m, 4H), 1.27 (s, 4H). The aqueous layer contained some deprotected pyrazole. Concentration and purification by RP-column (40% water in CH₃CN) provided 6-(1H-pyrazole-4-carbonyl)-2-(1-(trifluoromethyl)cyclopropane-1-carbonyl)-2,6-diazaspiro[3.4]octane-8-carbonitrile (0.58 g, 14%) as a white solid. LCMS m/z=368.1 [M+H]⁺.

### Step 8: (Z)—N′-hydroxy-6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboximidamide

To a solution of 6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carbonitrile (950 mg, 2.1 mmol) in ethanol (10 mL) was added a solution of hydroxylamine (50% in water, 1.0 mL). The reaction mixture was heated at reflux for 2 h then was cooled to room temperature and diluted with water (10 mL). The aqueous was extracted with EtOAc (30 mL×2) and the combined organic layers were washed with brine, dried over Na₂SO₄, filtered and concentrated to afford (Z)—N′-hydroxy-6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboximidamide (900 mg, 90%) as a white solid. LCMS m/z=485.2 [M+H]⁺; ¹H NMR (400 MHz, CDCl₃) δ 8.05 (s, 1H), 7.84 (s, 1H), 5.40 (d, J=7.7 Hz, 1H), 4.67 (s, 2H), 4.10-3.56 (m, 8H), 3.05-2.87 (m, 1H), 2.14-2.05 (m, 2H), 1.74-1.53 (m, 7H), 1.21 (s, 4H).

### Step 9: (1-(tetrahydro-2H-pyran-2-yl)-1H-pyrazol-4-yl)(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octan-6-yl)methanone

To a solution of 2-(3-(1-(trifluoromethyl)cyclopropyl)-1H-pyrazol-1-yl)propanoic acid (300 mg, 0.81 mmol) in a mixture of DMF and dioxane (3 mL/1 mL) was added EDCI (180 mg, 0.93 mmol) and (Z)—N′-hydroxy-6-(1-(tetrahydro-2H-pyran-2-yl)-1H-pyrazole-4-carbonyl)-2-(1-(trifluoromethyl)cyclopropane-1-carbonyl)-2,6-diazaspiro[3.4]octane-8-carboximidamide (300 mg, 0.62 mmol). The reaction was heated at 60° C. for 6 h then the temperature was increased to 100° C. and stirring continued for 14 h. The solvent was removed under vacuum and the residue obtained was purified by RP-column (40% water in acetonitrile) to afford methyl 2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoate (300 mg, 70%) as a white solid. LCMS m/z=697.0 [M+H]⁺; ¹H NMR (400 MHz, CDCl₃) δ 8.06 (s, 1H), 7.85 (s, 1H), 7.53-7.49 (m, 1H), 6.44 (s, 1H), 5.83-5.71 (m, 1H), 5.41 (d, J=8.8 Hz, 1H), 4.19-3.94 (m, 8H), 3.81-3.59 (m, 3H), 2.12-2.01 (m, 4H), 1.96 (d, J=7.1 Hz, 3H), 1.72-1.65 (m, 2H), 1.35-1.31 (m, 2H), 1.24-1.15 (m, 6H).

### Step 10: (6-(1H-pyrazole-4-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octan-2-yl)(1-(trifluoromethyl)cyclopropyl)methanone

To a solution of (1-(tetrahydro-2H-pyran-2-yl)-1H-pyrazol-4-yl)(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octan-6-yl)methanone (35 mg, 0.072 mmol) in DCM (1 mL) was added TFA (0.5 mL) and the reaction stirred at room temperature for 2 h. The solvent was removed under vacuum to afford (6-(1H-pyrazole-4-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octan-2-yl)(1-(trifluoromethyl)cyclopropyl)methanone (35 mg, 80%) which was used directly in the next step. LCMS m/z=613.2 [M+H]⁺.

### Step 11: methyl 5-(trifluoromethyl)-2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoate

To a solution of 6-(1H-pyrazole-4-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octan-2-yl)(1-(trifluoromethyl)cyclopropyl)methanone (150 mg, 0.24 mmol) in acetonitrile (2 mL) was added Cs₂CO₃ (399 mg, 21.22 mmol) and methyl 2-(bromomethyl)-5-(trifluoromethyl)benzoate (73 mg, 0.24 mmol). The reaction mixture was stirred at room temperature for 2 h then the solvent was removed and the residue obtained purified by RP-column (40% water in acetonitrile) to afford methyl 5-(trifluoromethyl)-2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoate (100 mg, 50%) as a white solid. LCMS m/z=829.1 [M+H]⁺; ¹H NMR (400 MHz, CDCl₃) δ 8.30 (s, 1H), 8.02 (s, 1H), 7.86 (d, J=8.1 Hz, 1H), 7.76-7.68 (m, 1H), 7.50 (d, J=2.5 Hz, 1H), 7.20-7.06 (m, 1H), 6.43 (s, 1H), 5.83 (s, 2H), 5.73 (q, J=7.3 Hz, 1H), 4.18-3.83 (m, 11H), 3.72-3.64 (m, 1H), 1.96 (d, J=7.4 Hz, 3H), 1.33-1.29 (m, 2H), 1.24-1.14 (m, 6H).

Table 36: The compounds listed in Table 36 were synthesized from according to the procedures outlined for I-219 using the appropriate commercially available reagents and/or intermediates described elsewhere.

TABLE 36

| Example | | | |
|---|---|---|---|
| Number | Compound | ¹HNMR | LCMS |
| I-215 | 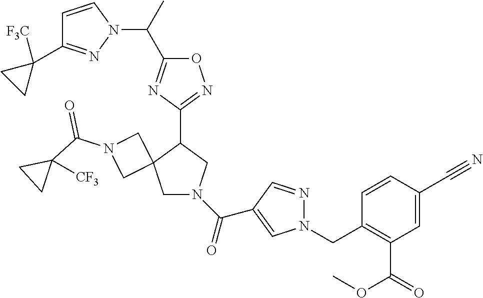 [SMILES: COC(=O)c1cc(C#N)ccc1Cn1cc(C(=O)N2CC(c3noc(C(C)n4ccc(C5(C(F)(F)F)CC5)n4)n3)C3(C2)CN(C(=O)C2(C(F)(F)F)CC2)C3)cn1] | ¹H NMR (400 MHz, CD₃OD) δ 8.36 − 8.26 (m, 2H), 8.00 − 7.92 (m, 1H), 7.88 − 7.83 (m, 1H), 7.79 − 7.74 (m, 1H), 7.07 − 7.01 (m, 1H), 6.41 (s, 1H), 5.96 − 5.89 (m, 1H), 5.89 − 5.85 (m, 2H), 4.50 − 3.83 (m, 12H), 1.97 − 1.91 (m, 3H), 1.30 − 1.16 (m, 8H). | m/z = 786.1 [M + H]⁺ |

### Synthesis of 5-(trifluoromethyl)-2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoic acid (I-218)

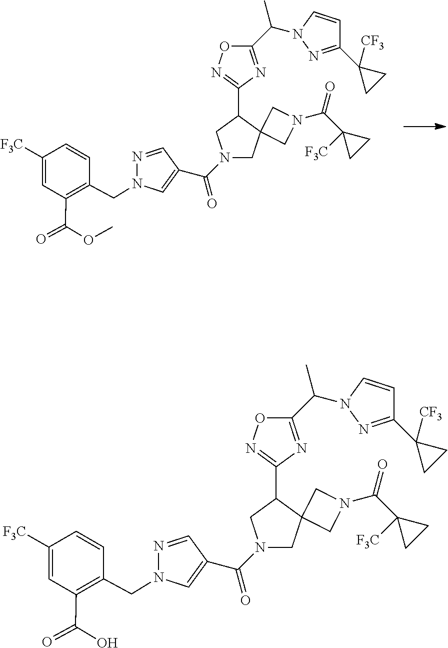 [SMILES: COC(=O)c1cc(C(F)(F)F)ccc1Cn1cc(C(=O)N2CC(c3noc(C(C)n4ccc(C5(C(F)(F)F)CC5)n4)n3)C3(C2)CN(C(=O)C2(C(F)(F)F)CC2)C3)cn1 ; CC(c1nc(C2CN(C(=O)c3cnn(Cc4ccc(C(F)(F)F)cc4C(=O)O)c3)CC23CN(C(=O)C2(C(F)(F)F)CC2)C3)no1)n1ccc(C2(C(F)(F)F)CC2)n1]

### Step 1: 5-(trifluoromethyl)-2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoic acid

To a solution of methyl 5-(trifluoromethyl)-2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoate (50 mg, 0.06 mmol) in methanol (1 mL) was added 10% aqueous NaOH (1.0 mL) and the reaction stirred at room temperature for 3 h. The reaction was diluted with water (10 mL) and extracted with ether (20 mL). The aqueous layer was collected, acidified with 1M HCl to pH˜2 and extracted with EtOAc (30 mL×3). The combined organic layers were washed with brine, dried over Na₂SO₄, filtered and concentrated. The residue was purified by RP-column (40% water in acetonitrile) to afford 5-(trifluoromethyl)-2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoic acid (25 mg, 50%) as a white solid. LCMS m/z=815.2 [M+H]⁺; ¹H NMR (400 MHz, CDCl₃) δ 8.30 (t, J=2.7 Hz, 2H), 7.87-7.69 (m, 2H), 7.57-7.50 (m, 1H), 7.34 (d, J=8.1 Hz, 1H), 6.43 (s, 1H), 5.86 (s, 2H), 5.76 (q, J=7.1 Hz, 1H), 4.16-3.94 (m, 8H), 3.73-3.65 (m, 1H), 1.97 (d, J=7.4 Hz, 3H), 1.34-1.29 (m, 2H), 1.24-1.13 (m, 6H).

Table 37: The compound listed in Table 37 were synthesized according to the procedures outlined for I-218 using the appropriate commercially available reagents and/or intermediates described elsewhere.

TABLE 37

| Example | | | |
|---|---|---|---|
| Number | Compound | ¹HNMR | LCMS |
| I-214 | 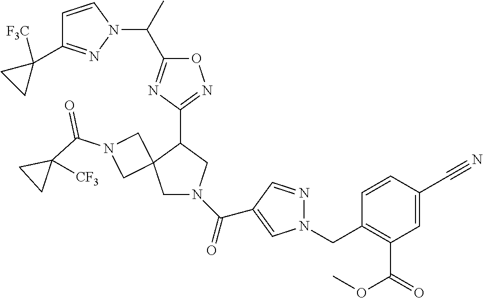 | ¹H NMR (400 MHz, CD₃OD) δ 8.32 − 8.28 (m, 1H), 7.99 − 7.92 (m, 1H), 7.85 − 7.66 (m, 2H), 7.17 − 6.97 (m, 1H), 6.41 (s, 1H), 5.96 − 5.82 (m, 3H), 4.66 − 4.50 (m, 1H), 4.34 − 3.82 (m, 9H), 1.97 − 1.92 (m, 3H), 1.37 − 1.26 (m, 4H), 1.22 − 1.17 (m, 4H). | m/z = 772.1 [M + H]⁺ |

### Synthesis of (1-(4-(trifluoromethyl)benzyl)-1H-pyrazol-4-yl)(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octan-6-yl)methanone (I-223)

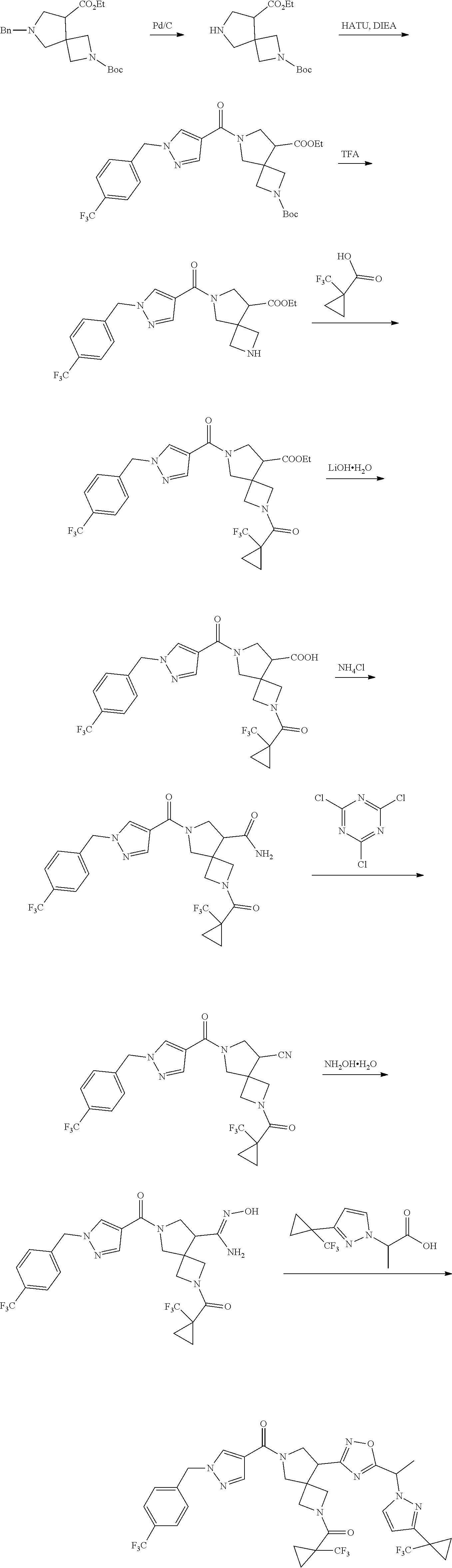

### Step 1: 2-(tert-butyl) 8-ethyl 2,6-diazaspiro[3.4]octane-2,8-dicarboxylate

To a solution of 2-(tert-butyl) 8-ethyl 6-benzyl-2,6-diazaspiro[3.4]octane-2,8-dicarboxylate (26.0 g, 69.4 mmol) in EtOAc (200 mL) was added 10% Pd/C (10.4 g) and the reaction heated at 50° C. under a H₂ atmosphere for 24 h. The catalyst was removed by filtration celite and the filtrate concentrated to afford 2-(tert-butyl) 8-ethyl 2,6-diazaspiro[3.4]octane-2,8-dicarboxylate (19.0 g, quant.) which was used directly in the next step. LCMS m/z=285.2 [M+H]⁺; ¹H NMR (400 MHz, DMSO-d₆) δ 4.19-4.00 (m, 2H), 3.84 (d, J=8.2 Hz, 1H), 3.76-3.56 (m, 3H), 3.12-2.81 (m, 5H), 1.36 (s, 9H), 1.19 (t, J=7.8, 6.4 Hz, 3H).

### Step 2: 2-(tert-butyl) 8-ethyl 6-(1-(4-(trifluoromethyl)benzyl)-1H-pyrazole-4-carbonyl)-2,6-diazaspiro[3.4]octane-2,8-dicarboxylate

To a solution of 1-(4-(trifluoromethyl)benzyl)-1H-pyrazole-4-carboxylic acid (16.0 g, 58.0 mmol) in DCM (150 mL) was added HATU (22.0 g, 58.0 mmol) and DIPEA (15.0 g, 116 mmol) and the mixture stirred at room temperature for 30 min. 2-(tert-butyl) 8-ethyl 2,6-diazaspiro[3.4]octane-2,8-dicarboxylate (15.0 g, 52.7 mmol) was added and stirring continued for 2 h. The mixture was diluted with water (100 mL) and extracted with DCM (150 mL×3). The combined organic layers were washed with brine, dried over Na₂SO₄, filtered and concentrated. The residue obtained was purified by column chromatography on silica gel (eluent: DCM/EtOAc=80/1) to afford 2-(tert-butyl) 8-ethyl 6-(1-(4-(trifluoromethyl)benzyl)-1H-pyrazole-4-carbonyl)-2,6-diazaspiro[3.4]octane-2,8-dicarboxylate (24 g, 86%) as a white solid. LCMS m/z=537.2 [M+H]⁺; ¹H NMR (400 MHz, DMSO-d₆) δ 8.42 (d, J=13.2 Hz, 1H), 7.85 (d, J=16.0 Hz, 1H), 7.72 (d, J=8.0 Hz, 2H), 7.44 (d, J=7.8 Hz, 2H), 5.48 (s, 2H), 4.24-3.63 (m, 11H), 1.37 (s, 9H), 1.23-1.17 (m, 3H).

### Step 3: ethyl 6-(1-(4-(trifluoromethyl)benzyl)-1H-pyrazole-4-carbonyl)-2,6-diazaspiro[3.4]octane-8-carboxylate

To a solution of 2-(tert-butyl) 8-ethyl 6-(1-(4-(trifluoromethyl)benzyl)-1H-pyrazole-4-carbonyl)-2,6-diazaspiro[3.4]octane-2,8-dicarboxylate (5.0 g, 9.3 mmol) in DCM (30 mL) was added TFA (15 mL) and the reaction mixture stirred at room temperature for 2 h. The solvent was removed under vacuum to afford ethyl 6-(1-(4-(trifluoromethyl)benzyl)-1H-pyrazole-4-carbonyl)-2,6-diazaspiro[3.4]octane-8-carboxylate (4.1 g, quant.) which was used directly in the next step. LCMS m % z=437.2 [M+H]f.

### Step 4: ethyl 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylate

To a solution of 1-(trifluoromethyl)cyclopropane-1-carboxylic acid (6.0 g, 38.8 mmol) in DCM (150 mL) was added HATU (14.8 g, 38.8 mmol) and DIPEA (13.7 g, 105.8 mmol) and the mixture stirred at room temperature for 30 min. Ethyl 6-(1-(4-(trifluoromethyl)benzyl)-1H-pyrazole-4-carbonyl)-2,6-diazaspiro[3.4]octane-8-carboxylate (15.4 g, 35.29 mmol) was added and stirring continued for 2 h. The mixture was diluted with water (150 mL) and extracted with DCM (200 mL×3). The combined organic layers were washed with brine, dried over Na₂SO₄, filtered and concentrated. The residue obtained was purified by column chromatography on silica gel (eluent: Pet.ether:EtOAc=1:1 to DCM/MeOH=80/1) to afford ethyl 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylate (18.5 g, 90%) as a white solid. LCMS m/z=573.1 [M+H]⁺; ¹H NMR (400 MHz, DMSO-d₆) δ 8.43 (d, J=6.4 Hz, 1H), 7.89 (d, 1H), 7.71 (d, J=8.0 Hz, 2H), 7.45 (d, J=8.0 Hz, 2H), 5.49 (s, 2H), 4.35-3.58 (m, 11H), 1.22-1.09 (m, 7H).

### Step 5: 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid

To a solution of ethyl 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylate (560 mg, 0.98 mmol) in a mixture of THE and H₂O (4 mL/1 mL) at 0° C. was added a solution of LiOH·H₂O (82 mg, 1.96 mmol) in H₂O (1 mL). The reaction was stirred at 0° C. for 2 h then was diluted with water (30 mL) and extracted with EtOAc (30 mL). The aqueous layer was collected, acidified with 1M HCl to pH˜2 and extracted with EtOAc (50 mL×2). The combined organic layers were washed with brine, dried over Na₂SO₄, filtered and concentrated to afford 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (350 mg, 66%) as a white solid. LCMS m/z=545.2 [M+H]⁺; ¹H NMR (400 MHz, DMSO-d₆) δ 8.42 (d, J=6.2 Hz, 1H), 7.86 (d, J=14.8 Hz, 1H), 7.72 (d, J=8.2 Hz, 2H), 7.44 (d, J=8.0 Hz, 2H), 5.49 (s, 2H), 4.38-3.62 (m, 9H), 1.38-1.29 (m, 1H), 1.21-1.10 (m, 3H).

### Step 6: 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide

To a solution of 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (350 mg, 0.64 mmol) in DMF (3.0 mL) was added HATU (366 mg, 0.96 mmol) and DIPEA (248 mg, 1.92 mmol) and the mixture stirred at room temperature for 30 min. NH₄Cl (103 mg, 1.92 mmol) was added and stirring continued for 3 h. The solvent was removed and the residue was purified by RP-column to afford 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (260 mg, 75%) as a yellow solid. LCMS m/z=544.2 [M+H]⁺; ¹H NMR (400 MHz, DMSO-d₆) δ 8.44-8.38 (m, 1H), 7.86 (d, J=10.4 Hz, 1H), 7.72 (d, J=8.2 Hz, 2H), 7.66 (s, 1H), 7.44 (d, J=8.0 Hz, 2H), 7.20 (d, J=20.2 Hz, 1H), 5.48 (s, 2H), 4.47-3.57 (m, 8H), 3.19-3.01 (m, 1H), 1.21-1.14 (m, 3H), 0.91-0.78 (m, 1H).

### Step 7: 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonitrile

To a solution of 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (2.0 g, 3.68 mmol) in DMF (20 mL) at 0° C. was added 2,4,6-trichloro-1,3,5-triazine (746 mg, 4.05 mmol) and the reaction stirred for 2 h. The reaction was diluted with water (80 mL) and extracted with EtOAc (100 mL×3). The combined organic layers were washed with water and brine, dried over Na₂SO₄, filtered and concentrated to afford 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonitrile (1.9 g, quant.) as a yellow solid. LCMS m/z=526.2 [M+H]⁺; ¹H NMR (400 MHz, DMSO-d₆) δ 8.44 (d, J=8.4 Hz, 1H), 7.97-7.83 (m, 1H), 7.73 (d, J=8.2 Hz, 2H), 7.45 (d, J=8.0 Hz, 2H), 5.49 (s, 2H), 4.39-3.64 (m, 9H), 1.28-1.13 (m, 4H).

### Step 8: (Z)—N′-hydroxy-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboximidamide

To a solution of 6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonitrile (8.7 g, 16.56 mmol) in EtOH (60 mL) was added NH₂OH·H₂O (15.0 g, 147.24 mmol) and the reaction heated at 50° C. for 3 h. The mixture was diluted with water (100 mL) and extracted with EtOAc (150 mL×3). The combined organic layers were washed with brine, dried over Na₂SO₄, filtered and concentrated to afford (Z)—N′-hydroxy-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboximidamide (9.2 g, quant) as a white solid. LCMS m/z=559.1 [M+H]⁺; ¹H NMR (400 MHz, DMSO-d₆) δ 9.14 (d, J=11.4 Hz, 1H), 8.38 (d, J=17.8 Hz, 1H), 7.84 (d, J=19.6 Hz, 1H), 7.72 (d, J=8.2 Hz, 2H), 7.44 (d, J=7.8 Hz, 2H), 5.61 (s, 2H), 5.48 (s, 2H), 4.39-3.53 (m, 8H), 3.09-2.94 (m, 1H), 1.25-1.13 (m, 4H).

### Step 9: (1-(4-(trifluoromethyl)benzyl)-1H-pyrazol-4-yl)(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octan-6-yl)methanone

To a solution of 2-(3-(1-(trifluoromethyl)cyclopropyl)-1H-pyrazol-1-yl)propanoic acid (39 mg, 0.16 mmol) in a mixture of DMF and dioxane (0.5 mL/0.2 mL) was added EDCI (40 mg, 0.21 mmol) and (Z)—N′-hydroxy-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboximidamide (80 mg, 0.14 mmol). The reaction mixture was heated at 60° C. for 3 h then the temperature increased to 110° C. and the reaction stirred overnight. The mixture was diluted with water (30 mL) and extracted with EtOAc (30 mL×3). The combined organic layers were washed with brine, dried over Na₂SO₄, filtered and concentrated. The residue obtained was purified by prep-HPLC to afford (1-(4-(trifluoromethyl)benzyl)-1H-pyrazol-4-yl)(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octan-6-yl)methanone (70 mg, 65%) as a white solid. LCMS m/z=771.1 [M+H]⁺; ¹H NMR (400 MHz, DMSO-d₆) δ 8.42 (d, J=10.6 Hz, 1H), 7.95-7.83 (m, 2H), 7.72 (d, J=7.8 Hz, 2H), 7.45 (d, J=7.8 Hz, 2H), 6.36 (s, 1H), 6.11-6.02 (m, 1H), 5.49 (s, 2H), 4.26-3.69 (m, 9H), 1.86-1.81 (m, 3H), 1.31-1.09 (m, 8H).

### Synthesis of (2-((S)-2,2-dimethylcyclopropane-1-carbonyl)-8-(6-(2-(thiazol-5-yl)phenoxy)benzo[d]oxazol-2-yl)-2,6-diazaspiro[3.4]octan-6-yl)(thiazol-5-yl)methanone (I-221)

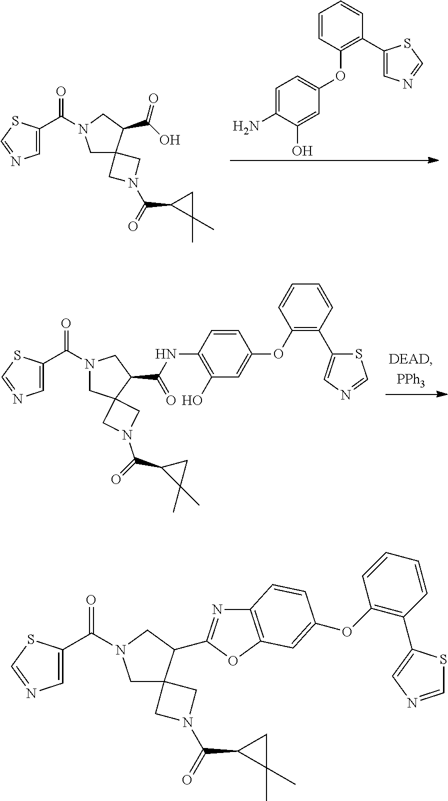 [SMILES: CC1(C)C[C@@H]1C(=O)N1CC2(CN(C(=O)c3cncs3)C[C@H]2C(=O)O)C1 ; Nc1ccc(Oc2ccccc2-c2cncs2)cc1O ; CC1(C)C[C@@H]1C(=O)N1CC2(CN(C(=O)c3cncs3)C[C@H]2C(=O)Nc2ccc(Oc3ccccc3-c3cncs3)cc2O)C1 ; CC1(C)C[C@@H]1C(=O)N1CC2(CN(C(=O)c3cncs3)CC2c2nc3ccc(Oc4ccccc4-c4cncs4)cc3o2)C1]

### Step 1: (S)-2-((S)-2,2-dimethylcyclopropane-1-carbonyl)-N-(2-hydroxy-4-(2-(thiazol-5-yl)phenoxy)phenyl)-6-(thiazole-5-carbonyl)-2,6-diazaspiro[3.4]octane-8-carboxamide

To a solution of (S)-2-((S)-2,2-dimethylcyclopropane-1-carbonyl)-6-(thiazole-5-carbonyl)-2,6-diazaspiro[3.4]octane-8-carboxylic acid (220 mg, 0.6 mmol) in DF (3 mL) was added 2-amino-5-(2-(thiazol-5-yl)phenoxy)phenol (187 mg, 0.7 mmol), EDCI (173 mg, 0.9 mmol), HOBt (122 mg, 0.9 mmol) and DIEA (233 mg, 1.8 mmol) and the reaction stirred at room temperature overnight. The mixture was diluted with water (20 mL) and extracted with EtOAc (30 mL×3). The combined organic layers were washed with brine, dried over Na₂SO₄, filtered and concentrated. The residue obtained was purified by prep-TLC (DCM/MeOH=10/1) to afford (S)-2-((S)-2,2-dimethylcyclopropane-1-carbonyl)-N-(2-hydroxy-4-(2-(thiazol-5-yl)phenoxy)phenyl)-6-(thiazole-5-carbonyl)-2,6-diazaspiro[3.4]octane-8-carboxamide (75 mg, 20%) as a yellow oil. LCMS m/z=630.1 [M+H]⁺; ¹H NMR (400 MHz, CDCl₃) δ 8.92 (s, 1H), 8.79-8.71 (m, 1H), 8.27 (s, 1H), 8.22 (s, 1H), 7.70 (d, J=7.4 Hz, 1H), 7.30 (d, J=6.6 Hz, 2H), 7.20 (t, J=7.8 Hz, 1H), 7.07-6.97 (m, 1H), 6.63-6.53 (m, 1H), 6.51-6.43 (m, 1H), 4.52-3.68 (m, 9H), 1.26 (s, 3H), 1.21-1.16 (m, 2H), 1.12-1.06 (m, 3H), 1.02-0.97 (m, 1H).

### Step 2: (2-((S)-2,2-dimethylcyclopropane-1-carbonyl)-8-(6-(2-(thiazol-5-yl)phenoxy)benzo[d]oxazol-2-yl)-2,6-diazaspiro[3.4]octan-6-yl)(thiazol-5-yl)

To a solution of (S)-2-((S)-2,2-dimethylcyclopropane-1-carbonyl)-N-(2-hydroxy-4-(2-(thiazol-5-yl)phenoxy)phenyl)-6-(thiazole-5-carbonyl)-2,6-diazaspiro[3.4]octane-8-carboxamide (40 mg, 0.1 mmol) in toluene (0.5 mL) was added PPh₃ (32 mg, 0.1 mmol) and DEAD (21 mg, 0.1 mmol). The mixture was heated at 120° C. for 2 h in a seal tube then the solvent removed and the residue obtained purified by prep-TLC (DCM/MeOH=15/1) to afford (2-((S)-2,2-dimethylcyclopropane-1-carbonyl)-8-(6-(2-(thiazol-5-yl)phenoxy)benzo[d]oxazol-2-yl)-2,6-diazaspiro[3.4]octan-6-yl)(thiazol-5-yl)methanone (26 mg, 65%) as a white solid. LCMS m/z=612.0 [M+H]⁺; ¹H NMR (400 MHz, CD₃OD) δ 9.18 (s, 1H), 8.95 (s, 1H), 8.42 (s, 1H), 8.32 (s, 1H), 7.91 (s, 1H), 7.68 (s, 1H), 7.43 (s, 1H), 7.31 (s, 1H), 7.15 (d, J=43.6 Hz, 3H), 4.43-3.96 (m, 9H), 1.39 (s, 1H), 1.17 (s, 3H), 0.94 (d, J=13.4 Hz, 3H), 0.70 (d, J=39.4 Hz, 1H), 0.57 (s, 1H).

### Synthesis of 2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoic acid (I-220)

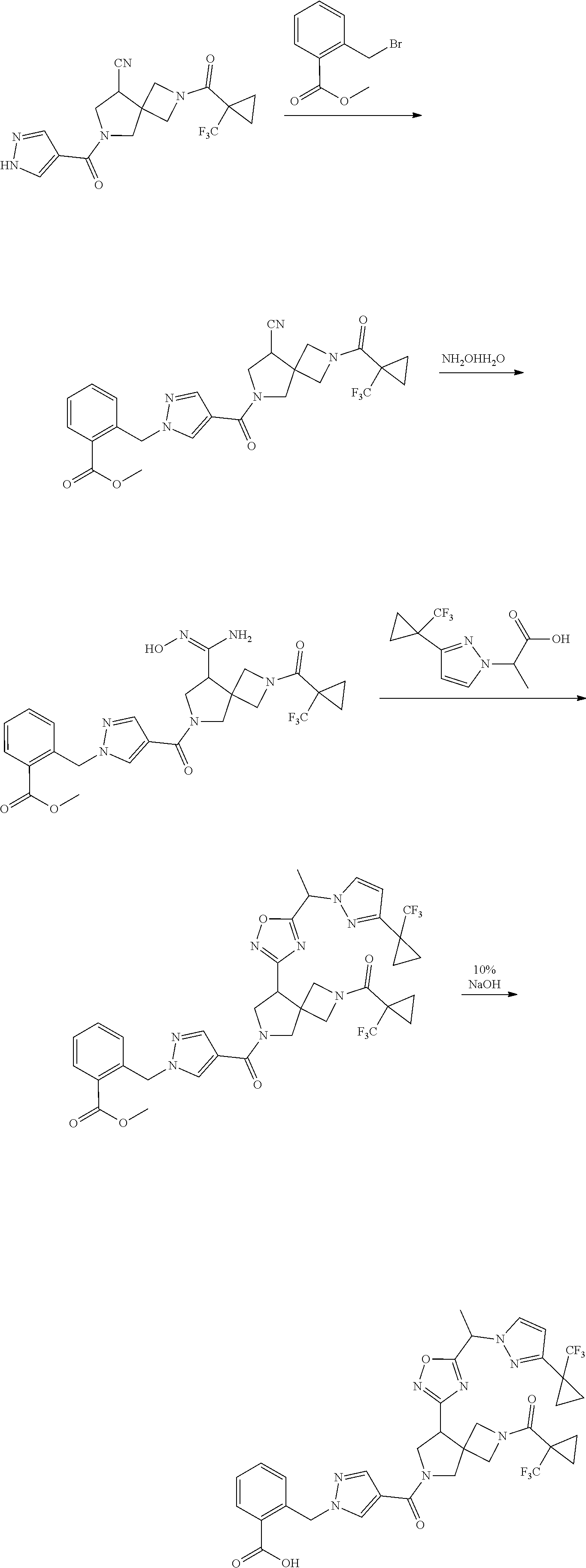

### Step 1: methyl 2-((4-(8-cyano-2-(1-(trifluoromethyl)cyclopropane-1-carbonyl)-2,6-diazaspiro[3.4]octane-6-carbonyl)-1H-pyrazol-1-yl)methyl)benzoate

To a solution of 6-(1H-pyrazole-4-carbonyl)-2-(1-(trifluoromethyl)cyclopropane-1-carbonyl)-2,6-diazaspiro[3.4]octane-8-carbonitrile (520 mg, 1.42 mmol) in acetonitrile (5 mL) was added potassium carbonate (392 mg, 2.84 mmol) and methyl 2-(bromomethyl)benzoate (357 mg, 1.56 mmol). The reaction was stirred at room temperature for 2 h then was diluted with water (30 mL), extracted with EtOAc (50 mL×2). The combined organic layers were washed with brine, dried over Na₂SO₄, filtered and concentrated. The residue obtained was purified by column chromatography on silica gel (eluent: DCM:MeOH=50:1) to afford methyl 2-((4-(8-cyano-2-(1-(trifluoromethyl)cyclopropane-1-carbonyl)-2,6-diazaspiro[3.4]octane-6-carbonyl)-1H-pyrazol-1-yl)methyl)benzoate (320 mg, 45%) as a white solid. LCMS m/z=516.1 [M+H]⁺; ¹H NMR (400 MHz, CDCl₃) δ 8.04 (dd, J=7.8, 1.4 Hz, 1H), 7.99 (s, 1H), 7.81 (s, 1H), 7.52 (td, J=7.6, 1.6 Hz, 1H), 7.44-7.39 (m, 1H), 7.15-7.09 (m, 1H), 5.77 (s, 2H), 4.68-3.92 (m, 8H), 3.92 (s, 3H), 3.36-3.24 (m, 1H), 1.29-1.25 (m, 4H).

### Step 2: methyl (E)-2-((4-(8-(N′-hydroxycarbamimidoyl)-2-(1-(trifluoromethyl)cyclopropane-1-carbonyl)-2,6-diazaspiro[3.4]octane-6-carbonyl)-1H-pyrazol-1-yl)methyl)benzoate

To a solution of methyl 2-((4-(8-cyano-2-(1-(trifluoromethyl)cyclopropane-1-carbonyl)-2,6-diazaspiro[3.4]octane-6-carbonyl)-1H-pyrazol-1-yl)methyl)benzoate (250 mg, 0.48 mmol) in ethanol (5.0 mL) was added hydroxylamine (50% in water, 1.0 mL) and the reaction heated at reflux for 2 h. th reaction was cooled to room temperature, diluted with water (20 mL) and extracted with EtOAc (50 mL×2). The combined organic layers were washed with brine, dried over Na₂SO₄, filtered and concentrated to afford methyl (E)-2-((4-(8-(N′-hydroxycarbamimidoyl)-2-(1-(trifluoromethyl)cyclopropane-1-carbonyl)-2,6-diazaspiro[3.4]octane-6-carbonyl)-1H-pyrazol-1-yl)methyl)benzoate (252 mg, 94%) as a white solid. LCMS m/z=549.1 [M+H]⁺; ¹H NMR (400 MHz, CDCl₃) δ 8.06-8.00 (m, 1H), 8.00-7.95 (m, 1H), 7.86-7.81 (m, 1H), 7.50 (t, J=7.9 Hz, 1H), 7.40 (t, J=7.6 Hz, 1H), 7.10-7.02 (m, 1H), 5.77 (s, 2H), 4.50-4.92 (m, 8H), 3.91 (s, 3H), 3.09-2.89 (m, 1H), 1.23-1.19 (m, 4H).

### Step 3: methyl 2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoate

To a solution of 2-(3-(1-(trifluoromethyl)cyclopropyl)-1H-pyrazol-1-yl)propanoic acid (49 mg, 0.20 mmol) in a mixture of DMF and dioxane (1 mL/0.2 mL) was added EDCI (52 mg, 0.27 mmol) and (methyl (E)-2-((4-(8-(N′-hydroxycarbamimidoyl)-2-(1-(trifluoromethyl)cyclopropane-1-carbonyl)-2,6-diazaspiro[3.4]octane-6-carbonyl)-1H-pyrazol-1-yl)methyl)benzoate (100 mg, 0.18 mmol). The mixture was heated at 60° C. for 6 h then heated to 100° C. and stirring continued for 14 h. The solvent was removed under vacuum and the residue obtained purified by RP-column (40% water in acetonitrile) to afford methyl 2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoate (90 mg, 66%) as a white solid. LCMS w/z=761.2 [M+H]⁺; ¹H NMR (400 MHz, CDCl₃) δ 8.07-8.01 (m, 2H), 7.87 (s, 1H), 7.56-7.48 (m, 2H), 7.45-7.38 (m, 1H), 7.14 (d, J=7.7 Hz, 1H), 6.43 (d, J=2.1 Hz, 1H), 5.80 (s, 2H), 5.79-5.71 (m, 1H), 4.47-3.99 (m, 7H), 3.92 (s, 3H), 3.72-3.63 (m, 1H), 3.10-2.86 (m, 1H), 1.96 (dd, J=7.2, 1.2 Hz, 3H), 1.33-1.14 (m, 8H).

### Step 4: 2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoic acid

To a solution of methyl 2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoate (65 mg, 0.09 mmol) in methanol (1.0 mL) was added 10% NaOH aqueous solution (0.1 mL) and the reaction stirred at room temperature for 3 h. The mixture was diluted with water (30 mL) and extracted with EtOAc (30 mL). The aqueous layer was collected, acidified with 1M HCl to pH˜2 and extracted with EtOAc (150 mL×3). The combined organic layers were washed with brine, dried over Na₂SO₄, filtered and concentrated. The residue obtained was purified by RP-column (40% water in acetonitrile) to afford 2-((4-(2-(1-(trifluoromethyl)cyclopropane-1-carbonyl)-8-(5-(1-(3-(1-(trifluoromethyl)cyclopropyl)-1H-pyrazol-1-yl)ethyl)-1,2,4-oxadiazol-3-yl)-2,6-diazaspiro[3.4]octane-6-carbonyl)-1H-pyrazol-1-yl)methyl)benzoic acid (35 mg, 42%) as a white solid. LCMS m/z=747.1 [M+H]⁺; ¹H NMR (400 MHz, CDCl₃) δ 8.20 (s, 1H), 8.04 (d, J=7.7 Hz, 1H), 7.87 (s, 1H), 7.56-7.48 (m, 2H), 7.45-7.37 (m, 1H), 7.26-7.23 (m, 1H), 6.43 (s, 1H), 5.80 (s, 2H), 5.79-5.72 (m, 1H), 4.46-3.86 (m, 8H), 3.74-3.62 (m, 1H), 1.96 (d, J=7.0 Hz, 3H), 1.35-1.30 (m, 2H), 1.24-1.21 (m, 2H), 1.21-1.09 (m, 4H).

### Synthesis of 2-(4-cyclohexylphenyl)-3-oxo-3-(4-(thiazol-2-yl)piperidin-1-yl)propanehydrazide

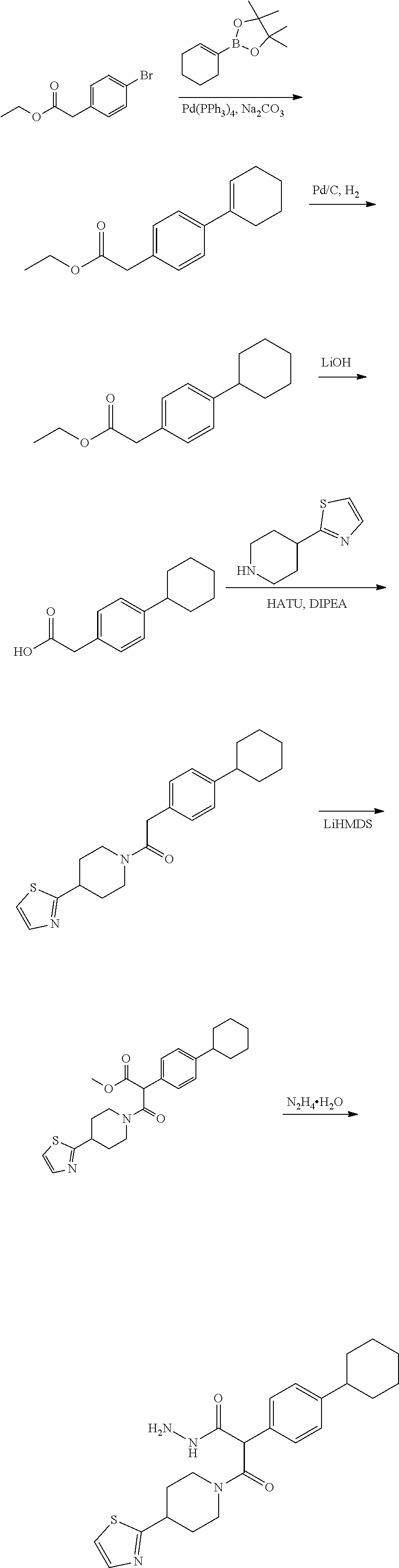

### Step 1: ethyl 2-(2′,3′,4′,5′-tetrahydro-[1,1′-biphenyl]-4-yl)acetate

To a solution of ethyl 2-(4-bromophenyl)acetate (5 g, 0.02 mol) in a mixture of ethylene glycol dimethyl ether (12 mL) and water (6 mL) was added 2-(cyclohex-1-en-1-yl)-4,4,5,5-tetramethyl-1,3,2-dioxaborolane (5.1 g, 0.02 mol), Na₂CO₃ (6.4 g, 0.06 mol) and Pd(PPh₃)₄ (2.4 g, 0.002 mol). The reaction was heated at 100° C. under a N₂ atmosphere overnight then was diluted with water (200 mL) and extracted with EtOAc (200 mL×3). The combined organic layers were dried over Na₂SO₄, filtered and concentrated. The residue was purified by column chromatography on silica gel (eluent: Pet. Ether:EtOAc=80:1, v/v) to afford ethyl 2-(2′,3′,4′,5′-tetrahydro-[1,1′-biphenyl]-4-yl)acetate (1.6 g, 32%) as a colorless oil. ¹H NMR (400 MHz, DMSO-d6) δ 7.34 (d, J=8.0 Hz, 2H), 7.19 (d, J=8.0 Hz, 2H), 6.13 (tt, J=3.8, 1.6 Hz, 1H), 4.07 (q, J=7.0 Hz, 2H), 3.62 (s, 2H), 2.34 (dp, J=6.4, 2.4 Hz, 2H), 2.16 (tt, J=6.0, 3.2 Hz, 2H), 1.71 (m, 2H), 1.60 (m, 2H), 1.18 (t, J=7.0 Hz, 3H).

### Step 2: ethyl 2-(4-cyclohexylphenyl)acetate

To a solution of ethyl 2-(2′,3′,4′,5′-tetrahydro-[1,1′-biphenyl]-4-yl)acetate (1.65 g, 6.76 mmol) in MeOH (10 mL) was added 10% Pd/C (660 mg) and the reaction stirred at 35° C. under H₂ atmosphere overnight. The catalyst was removed by filtration through Celite and the filtrate concentrated to afford the ethyl 2-(4-cyclohexylphenyl)acetate (1.4 g crude, 84%) as a colorless oil, which was used in the next step directly. ¹H NMR (400 MHz, DMSO-d6) δ 7.16 (d, J=1.0 Hz, 4H), 4.10-4.03 (m, 2H), 3.59 (s, 2H), 2.46 (s, 1H), 1.77 (m, 4H), 1.73-1.67 (m, 1H), 1.41-1.31 (m, 4H), 1.25 (m, 1H), 1.20-1.15 (m, 3H).

### Step 3: 2-(4-cyclohexylphenyl)acetic acid

To a solution of ethyl 2-(4-cyclohexylphenyl)acetate (1.55 g, 6.30 mmol) in a mixture of THF, water and MeOH (2.0 mL/0.5 mL/0.5 mL) was added LiOH (793 mg, 18.89 mmol) and the reaction stirred at room temperature for 2 h. The mixture was diluted with water (30 mL) and extracted with EtOAc (30 mL). The aqueous layer was collected, acidified to pH˜2 with 1M HCl and extracted with EtOAc (40 mL×3). The combined organic layers were washed with brine, dried over Na₂SO₄, filtered and concentrated to afford 2-(4-cyclohexylphenyl)acetic acid (1.2 g, 87) as a white solid. ¹H NMR (400 MHz, DMSO-d6) δ 12.26 (s, 1H), 7.15 (s, 4H), 3.50 (s, 2H), 2.45 (d, J=10.0 Hz, 1H), 1.81-1.65 (m, 5H), 1.43-1.21 (m, 5H).

### Step 4: 2-(4-cyclohexylphenyl)-1-(4-(thiazol-2-yl)piperidin-1-yl)ethan-1-one

To a solution of 2-(4-cyclohexylphenyl)acetic acid (270 mg, 1.24 mmol) in DCM (4 mL) was added HATU (471 mg, 1.24 mmol) and DIPEA (481 mg, 3.72 mmol) and the mixture stirred at room temperature for 20 min. 2-(piperidin-4-yl)thiazole (250 mg, 1.49 mmol) was added and stirring continued for 2 h. The mixture was diluted with water (30 mL) and extracted with DCM (30 mL×3). The combined organic layers were washed with brine, dried over Na₂SO₄, filtered and concentrated. The residue obtained was purified by RP-Column (eluent: MeCN:H₂O=60%:40%) to afford 2-(4-cyclohexylphenyl)-1-(4-(thiazol-2-yl)piperidin-1-yl)ethan-1-one (210 mg, 46%) as a yellow solid. LCMS m/z=369.10 [M+H]⁺; ¹H NMR (400 MHz, DMSO-d6) δ 7.70 (d, J=3.2 Hz, 1H), 7.59 (d, J=3.2 Hz, 1H), 7.14 (s, 4H), 4.43 (d, J=13.2 Hz, 1H), 4.01 (d, J=13.6 Hz, 1H), 3.68 (d, J=2.2 Hz, 2H), 3.25 (m, 1H), 3.18-3.10 (m, 1H), 2.74 (m, 1H), 2.47-2.41 (m, 1H), 2.04-1.94 (m, 2H), 1.76 (dd, J=10.2, 5.8 Hz, 4H), 1.69 (d, J=12.6 Hz, 1H), 1.41 (m, 6H), 1.21 (m, 1H).

### Step 5: methyl 2-(4-cyclohexylphenyl)-3-oxo-3-(4-(thiazol-2-yl)piperidin-1-yl)propanoate

To a solution of 2-(4-cyclohexylphenyl)-1-(4-(thiazol-2-yl)piperidin-1-yl)ethan-1-one (150 mg, 0.41 mol) and dimethyl carbonate (81 mg, 0.61 mol) in anhydrous THF (2 mL) at 0° C. under a N₂ atmosphere was added LiHMDS (1.0 mol in THF) (2 mL, 2.04 mmol). The reaction was stirred for 2.5 h then was diluted with water (15 mL) and extracted with EtOAc (15 mL×2). The combined organic layers were washed with brine, dried over Na₂SO₄, filtered and concentrated. The residue was purified by prep-TLC (eluent: Pet.Ether:EtOAc=1:2) to afford methyl 2-(4-cyclohexylphenyl)-3-oxo-3-(4-(thiazol-2-yl)piperidin-1-yl)propanoate (85 mg, 49%) as a white solid. LCMS m/z=427.2 [M+H]⁺; ¹H NMR (400 MHz, DMSO-d6) δ 7.68 (m, 1H), 7.58 (m, 1H), 7.26 (dd, J=8.2, 3.4 Hz, 2H), 7.19 (d, J=8.0 Hz, 2H), 5.30 (d, J=12.0 Hz, 1H), 4.39 (t, J=11.4 Hz, 1H), 3.92 (m, 1H), 3.63 (s, 3H), 3.22 (m, 1H), 2.82-2.68 (m, 1H), 2.01 (m, 2H), 1.83-1.54 (m, 7H), 1.43-1.16 (m, 7H).

### Step 6: 2-(4-cyclohexylphenyl)-3-oxo-3-(4-(thiazol-2-yl)piperidin-1-yl)propanehydrazide

To a solution of methyl 2-(4-cyclohexylphenyl)-3-oxo-3-(4-(thiazol-2-yl)piperidin-1-yl)propanoate (50 mg, 0.12 mmol) in EtOH (1 mL) was added 98% hydrazine hydrate (0.5 mL) and the reaction stirred at room temperature for 1 h. The mixture was diluted with water (10 mL) and extracted with EtOAc (10 mL×2). The combined organic layers were washed with brine, dried over Na₂SO₄, filtered and concentrated to afford 2-(4-cyclohexylphenyl)-3-oxo-3-(4-(thiazol-2-yl)piperidin-1-yl)propanehydrazide (50 mg, 100%) as a white solid. LCMS m/z=427.2 [M+H]⁺; ¹H NMR (400 MHz, DMSO-d6) δ 9.13 (m, 1H), 7.64 (m, 2H), 7.21 (d, J=8.0 Hz, 2H), 7.14 (d, J=8.0 Hz, 2H), 4.81 (d, J=4.6 Hz, 1H), 4.41 (d, J=13.0 Hz, 1H), 4.23 (s, 2H), 3.92 (s, 1H), 3.25 (s, 1H), 3.09 (m, 1H), 2.81-2.69 (m, 1H), 2.46 (s, 1H), 2.01 (s, 1H), 1.87-1.66 (m, 7H), 1.56 (m, 1H), 1.36 (s, 5H), 1.13-1.01 (m, 1H).

### Synthesis of 3-(benzyloxy)-2-(3-cyclohexylphenyl)propanehydrazide

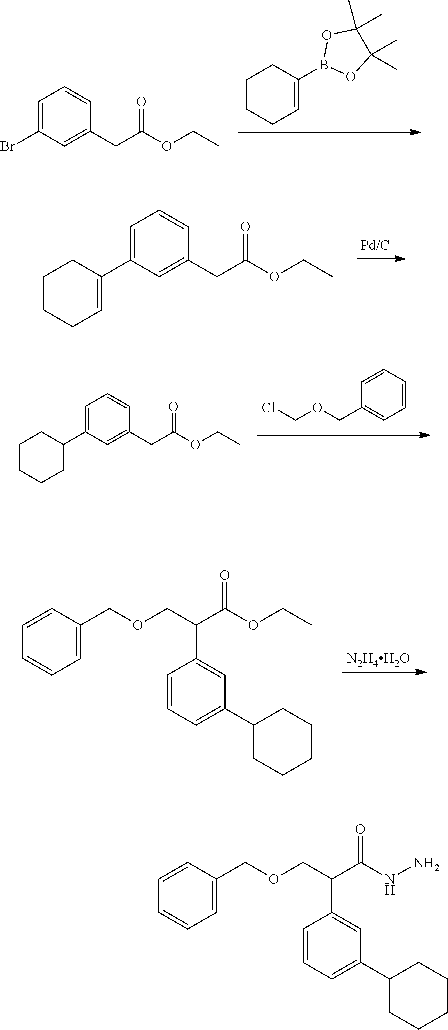

### Step 1: ethyl 2-(2′,3′,4′,5′-tetrahydro-[1,1′-biphenyl]-3-yl)acetate

To a solution of ethyl 2-(3-bromophenyl)acetate (500 mg, 2.1 mmol) in a mixture of dioxane (4 mL) and water (1 mL) under a N₂ atmosphere was added Pd(PPh₃)₄ (119 mg, 0.1 mmol), 2-(cyclohex-1-en-1-yl)-4,4,5,5-tetramethyl-1,3,2-dioxaborolane (472 mg, 2.2 mmol) and K₂CO₃ (570 mg, 4.1 mmol). The reaction was heated at 100° C. for 5 h then was diluted with water (20 mL) and extracted with EtOAc (30 mL×3). The combined organic layers were washed with brine, dried over Na₂SO₄, filtered and concentrated. The residue was purified by column chromatography on silica gel (eluent: Petroleum ether:EtOAc=20:1) to afford ethyl 2-(2′,3′,4′,5′-tetrahydro-[1,1′-biphenyl]-3-yl)acetate (320 mg, 64%) as a colorless oil. ¹H NMR (400 MHz, Chloroform-d) δ 7.31-7.22 (m, 3H), 7.16-7.10 (m, 1H), 6.14-6.08 (m, 1H), 4.14 (q, J=7.1 Hz, 2H), 3.59 (s, 2H), 2.43-2.35 (m, 2H), 2.24-2.15 (m, 2H), 1.81-1.72 (m, 2H), 1.70-1.61 (m, 2H), 1.25 (t, J=7.1 Hz, 3H).

### Step 2: ethyl 2-(3-cyclohexylphenyl)acetate

To a solution of ethyl 2-(2′,3′,4′,5′-tetrahydro-[1,1′-biphenyl]-3-yl)acetate (320 mg, 1.3 mmol) in EtOH (4 mL) was added 10% Pd/C (10 mg) and the reaction stirred under H₂ atmosphere for 2 h. The catalyst was removed by filtration through Celite and the filtrate concentrated to afford ethyl 2-(3-cyclohexylphenyl)acetate (320 mg, quant) as a colorless oil. ¹HNMR (400 MHz, Chloroform-d) δ 7.30-7.25 (m, 1H), 7.18-7.11 (m, 3H), 4.19 (q, J=7.1 Hz, 2H), 3.62 (s, 2H), 2.58-2.47 (m, 1H), 1.96-1.82 (m, 4H), 1.81-1.74 (m, 1H), 1.52-1.22 (m, 8H).

### Step 3: ethyl 3-(benzyloxy)-2-(3-cyclohexylphenyl)propanoate

To a solution of ethyl 2-(3-cyclohexylphenyl)acetate (5 g, 20.3 mmol) and ((chloromethoxy)methyl)benzene (3.4 g, 24.4 mmol) in dry THF (50 mL) at −78° C. was added LiHMDS (1 M in THF, 40.6 mL, 40.6 mmol) dropwise. The reaction was stirred at room temperature for 2 h then was diluted with water (30 mL) and extracted with EtOAc (50 mL×3). The combined organic layers were washed with brine, dried over Na₂SO₄, filtered and concentrated. The mixture was purified by RP-column to afford ethyl 3-(benzyloxy)-2-(3-cyclohexylphenyl)propanoate (4.7 g, 64%) as a colorless oil. ¹H NMR (400 MHz, Chloroform-d) δ 7.36-7.20 (m, 7H), 7.16-7.10 (m, 3H), 4.56 (q, J=12.2 Hz, 2H), 4.26-4.03 (m, 3H), 3.93-3.87 (m, 1H), 3.69-3.63 (m, 1H), 2.52-2.43 (m, 1H), 1.91-1.71 (m, 6H), 1.47-1.32 (m, 4H), 1.23 (t, J=7.1 Hz, 3H).

### Step 4: 3-(benzyloxy)-2-(3-cyclohexylphenyl)propanehydrazide

To a solution of ethyl 3-(benzyloxy)-2-(3-cyclohexylphenyl)propanoate (1 g, 2.7 mmol) in MeOH (3.0 mL) was added hydrazine hydrate (3.0 mL). The reaction was heated at 100° C. for 1 h in a sealed tube then was diluted with water (50 mL) and extracted with EtOAc (30 mL×3). The combined organic layers were washed with brine, dried over Na₂SO₄, filtered and concentrated. The residue was purified by RP-column to afford 3-(benzyloxy)-2-(3-cyclohexylphenyl)propanehydrazide (500 mg, 52%) as a yellow oil. LCMS m/z=352.7 [M+H]⁺.

### Synthesis of 2-amino-5-(2-(thiazol-5-yl)phenoxy)phenol

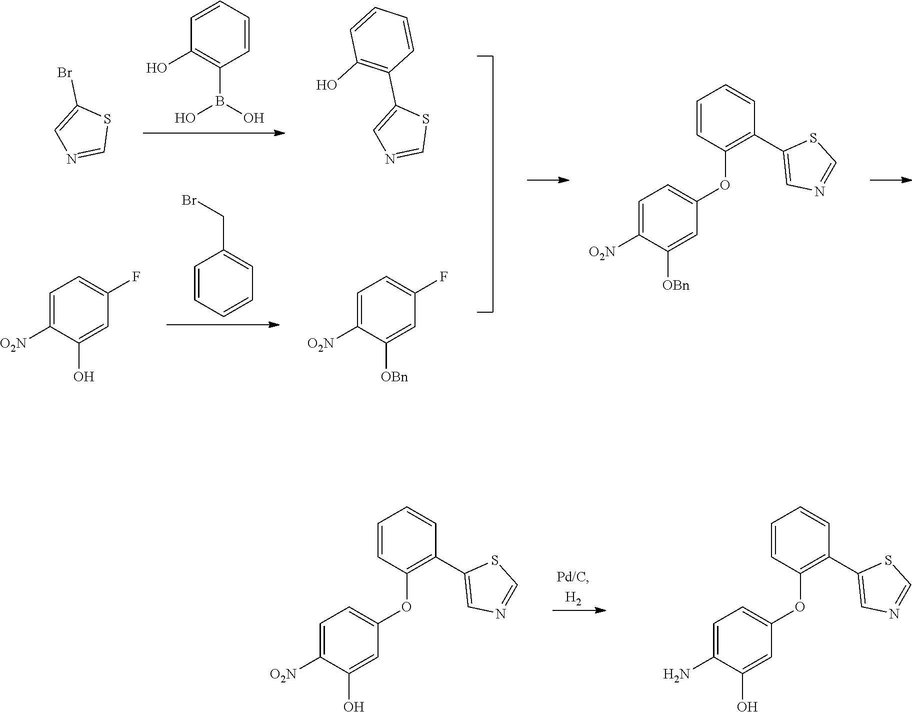

### Step 1: 2-(thiazol-5-yl)phenol

To a solution of 5-bromothiazole (1 g, 6.1 mmol) in a mixture of dioxane and water (9/3 mL) was added (2-hydroxyphenyl)boronic acid (919 mg, 6.7 mmol), Pd(PPh₃)₄ (352 mg, 0.3 mmol) and K₃PO₄ (3.9 g, 18.3 mmol). The reaction was heated at 80° C. under N₂ atmosphere overnight then was diluted with water (30 mL) and extracted with EtOAc (50 mL×3). The combined organic layers were washed with brine, dried over Na₂SO₄, filtered and concentrated to afford 2-(thiazol-5-yl)phenol (964 mg, 88%) as a white solid. LCMS m/z=178.1 [M+H]⁺; ¹H NMR (400 MHz, DMSO-d₆) δ 10.41 (s, 1H), 9.02 (s, 1H), 8.37 (s, 1H), 7.74-7.65 (m, 1H), 7.26-7.14 (m, 1H), 7.01-6.94 (m, 1H), 6.92-6.85 (m, 1H).

### Step 2: 2-(benzyloxy)-4-fluoro-1-nitrobenzene

To a solution of 5-fluoro-2-nitrophenol (1 g, 6.4 mmol) in DMF (10 mL) was added (bromomethyl)benzene (1.2 g, 7.0 mmol) and K₂CO₃ (1.1 g, 7.7 mmol). The mixture was stirred at 60° C. for 3 h then was diluted with water (30 mL) and extracted with EtOAc (50 mL×3). The combined organic layers were washed with brine, dried over Na₂SO₄, filtered and concentrated to afford 2-(benzyloxy)-4-fluoro-1-nitrobenzene (1.2 g, 86%) as a yellow oil. LCMS m/z=247.2 [M+H]⁺; ¹H NMR (400 MHz, CDCl₃) δ 8.00-7.93 (m, 1H), 7.49-7.32 (m, 5H), 6.83 (d, J=10.2 Hz, 1H), 6.74 (d, J=0.8 Hz, 1H), 5.23 (s, 2H).

### Step 3: 5-(2-(3-(benzyloxy)-4-nitrophenoxy)phenyl)thiazole

To a solution of 2-(benzyloxy)-4-fluoro-1-nitrobenzene (1.1 g, 4.7 mmol) in DMF (10 mL) was added 2-(thiazol-5-yl)phenol (832 mg, 4.7 mmol) and K₂CO₃ (650 mg, 4.7 mmol). The reaction was heated at 100° C. overnight then was diluted with water (30 mL) and extracted with EtOAc (30 mL×3). The combined organic layers were washed with brine, dried over Na₂SO₄, filtered and concentrated. The residue obtained was purified by silica gel column (Pet. ether/EtOAc=2/1) to afford 5-(2-(3-(benzyloxy)-4-nitrophenoxy)phenyl)thiazole (1.6 g, 85%) as a yellow oil. LCMS m/z=404.4 [M+H]⁺; ¹H NMR (400 MHz, CDCl₃) δ 8.75 (s, 1H), 8.14 (s, 1H), 7.91 (d, J=9.2 Hz, 1H), 7.80-7.70 (m, 1H), 7.42-7.29 (m, 7H), 7.06 (d, J=7.8 Hz, 1H), 6.62 (d, J=2.2 Hz, 1H), 6.52-6.42 (m, 1H), 5.15 (s, 2H).

### Step 4: 2-nitro-5-(2-(thiazol-5-yl)phenoxy)phenol

To a solution of 5-(2-(3-(benzyloxy)-4-nitrophenoxy)phenyl)thiazole (1.2 g, 3.0 mmol) was added HBr (48% in AcOH, 10 mL) and the reaction heated at 100° C. overnight. The mixture was diluted with water (30 mL) and extracted with EtOAc (30 mL×4). The combined organic layers were washed with brine, dried over Na₂SO₄, filtered and concentrated to afford 2-nitro-5-(2-(thiazol-5-yl)phenoxy)phenol (600 mg, 64%) as a yellow oil. LCMS m/z=314.5 [M+H]⁺; ¹H NMR (400 MHz, CD₃OD) δ 8.98 (s, 1H), 8.24 (s, 1H), 8.12 (d, J=9.4 Hz, 1H), 7.94-7.89 (m, 1H), 7.49-7.41 (m, 2H), 7.30-7.26 (m, 1H), 6.66-6.60 (m, 1H), 6.50 (d, J=2.6 Hz, 1H).

### Step 5: 2-amino-5-(2-(thiazol-5-yl)phenoxy)phenol

To a solution of 2-nitro-5-(2-(thiazol-5-yl)phenoxy)phenol (600 mg, 1.9 mmol) in EtOAc (10 mL) was added 10% Pd/C (300 mg, 1.2 mmol) and the reaction stirred at 45° C. overnight under a hydrogen atmosphere. The catalyst was removed by filtration through Celite and the filtrate concentrated to afford 2-amino-5-(2-(thiazol-5-yl)phenoxy)phenol (475 mg, 87%) as a yellow oil. LCMS m/z=284.6 [M+H]⁺; ¹H NMR (400 MHz, CD₃OD) δ 8.97 (s, 1H), 8.33 (s, 1H), 7.87-7.79 (m, 1H), 7.37-7.30 (m, 1H), 7.24-7.17 (m, 1H), 7.02-6.94 (m, 1H), 6.86 (d, J=8.4 Hz, 1H), 6.49 (d, J=2.6 Hz, 1H), 6.44-6.37 (m, 1H).

### Synthesis of 2-(3-(2-oxabicyclo[2.2.2]octan-4-yl)-1H-pyrazol-1-yl)propanoic acid

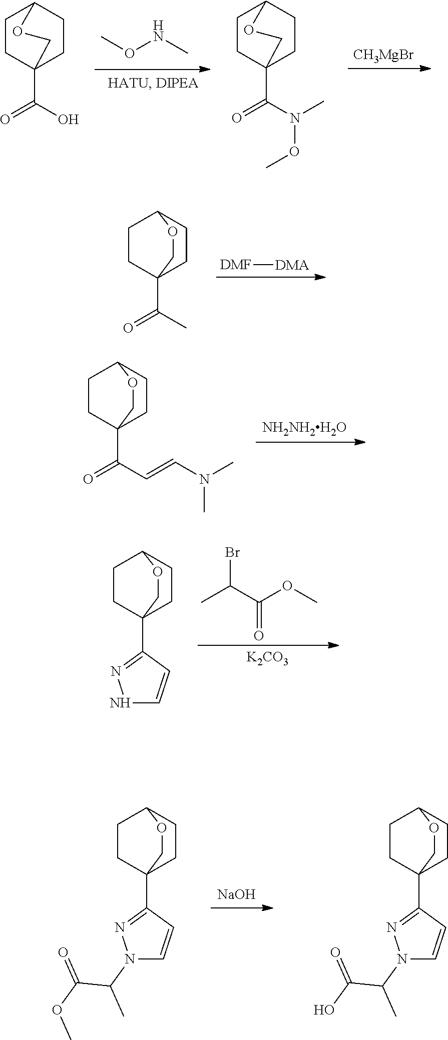

### Step 1: N-methoxy-N-methyl-2-oxabicyclo[2.2.2]octane-4-carboxamide

To a solution of N,O-dimethylhydroxylamine hydrochloride (350 mg, 3.52 mmol) in DCM (8 mL) was added HATU (1.46 g, 3.84 mmol) and the mixture was stirred at room temperature for 30 min. 2-oxabicyclo[2.2.2]octane-4-carboxylic acid (500 mg, 3.2 mmol) and DIPEA (1.65 g, 12.8 mmol) were added and stirring continued for 2 h. The mixture was diluted with water (20 mL) and extracted with DCM (50 mL×3). The combined organic layers were washed with brine, dried over Na₂SO₄, filtered and concentrated to afford N-methoxy-N-methyl-2-oxabicyclo[2.2.2]octane-4-carboxamide (700 mg, 100%) as a yellow oil. LCMS m/z=200.2 [M+H]⁺; ¹H NMR (400 MHz, DMSO-d₆) δ 3.83 (t, J=1.4 Hz, 2H), 3.69 (m, 1H), 3.65 (s, 3H), 3.05 (s, 3H), 2.10-2.01 (m, 2H), 1.93-1.84 (m, 2H), 1.78 (m, 2H), 1.59 (m, 2H).

### Step 2: 1-(2-oxabicyclo[2.2.2]octan-4-yl)ethan-1-one

To a solution of N-methoxy-N-methyl-2-oxabicyclo[2.2.2]octane-4-carboxamide (720 mg, 3.6 mmol) in THF (40 mL) at −78° C. under a N₂ atmosphere was added CH₃MgBr (1 M, 36 mL). The reaction was allowed to warm to room temperature and was stirred overnight. The reaction was diluted with NH₄Cl(aq) (50 mL) and extracted with EtOAc (50 mL×3). The combined organic layers were washed with brine, dried over Na₂SO₄, filtered and concentrated to afford 1-(2-oxabicyclo[2.2.2]octan-4-yl)ethan-1-one (460 mg, 82%) as a yellow oil. ¹H NMR (400 MHz, Chloroform-d) δ 3.92 (s, 2H), 3.82 (m, 1H), 2.12-2.08 (m, 1H), 2.08 (s, 3H), 2.07-2.04 (m, 1H), 1.86 (dd, J=9.0, 6.8 Hz, 4H), 1.69-1.61 (m, 2H).

### Step 3: (E)-1-(2-oxabicyclo[2.2.2]octan-4-yl)-3-(dimethylamino)prop-2-en-1-one

To a solution of 1-(2-oxabicyclo[2.2.2]octan-4-yl)ethan-1-one (160 mg, 1.04 mmol) in DMF (2 mL) in sealed tube was added 1,1-dimethoxy-N,N-dimethylmethanamine (0.5 mL) and the reaction was heated at 100° C. overnight. The mixture was diluted with water (30 mL) and extracted with EtOAc (20 mL×3). The combined organic layers were washed with brine, dried over Na₂SO₄, filtered and concentrated to afford (E)-1-(2-oxabicyclo[2.2.2]octan-4-yl)-3-(dimethylamino)prop-2-en-1-one (76 mg, 35%) as a yellow oil. LCMS m/z=210.2 [M+H]⁺; ¹H NMR (400 MHz, Chloroform-d) δ 7.63 (d, J=12.0 Hz, 1H), 5.06 (d, J=12.2 Hz, 1H), 3.95 (t, J=1.4 Hz, 2H), 3.83 (m, 1H), 3.04 (m, 3H), 2.84 (m, 3H), 2.06-2.02 (m, 2H), 1.95-1.89 (m, 2H), 1.86 (m, 2H), 1.65-1.61 (m, 2H).

### Step 4: 3-(2-oxabicyclo[2.2.2]octan-4-yl)-1H-pyrazole

To a solution of (E)-1-(2-oxabicyclo[2.2.2]octan-4-yl)-3-(dimethylamino)prop-2-en-1-one (215 mg, 1.03 mmol) in EtOH (4 mL) was added hydrazine hydrate (257 mg, 5.14 mmol) and the reaction heated at 80° C. for 3 h. The mixture was concentrated, diluted with water (30 mL) and extracted with EtOAc (20 mL×3). The combined organic layers were washed with brine, dried over Na₂SO₄, filtered and concentrated to afford 3-(2-oxabicyclo[2.2.2]octan-4-yl)-1H-pyrazole (100 mg, 54%) as a yellow oil. LCMS m/z=179.2 [M+H]⁺; ¹H NMR (400 MHz, Chloroform-d) δ 7.49 (d, J=2.2 Hz, 1H), 6.12 (d, J=2.2 Hz, 1H), 3.97 (t, J=1.6 Hz, 2H), 3.90 (m, 1H), 2.17 (m, 2H), 2.06 (m, 2H), 1.98-1.91 (m, 2H), 1.75 (m, 2H).

### Step 5: methyl 2-(3-(2-oxabicyclo[2.2.2]octan-4-yl)-1H-pyrazol-1-yl)propanoate

To a solution of 3-(2-oxabicyclo[2.2.2]octan-4-yl)-1H-pyrazole (70 mg, 0.39 mmol) in DMF (2 mL) was added K₂CO₃ (163 mg, 1.18 mmol) and methyl 2-bromopropanoate (197 mg, 1.18 mmol) and the reaction heated at 70° C. for 3 h. The mixture was concentrated, diluted with water (30 mL) and extracted with EtOAc (20 mL×3). The combined organic layers were washed with brine, dried over Na₂SO₄, filtered and concentrated. The residue was purified by prep-TLC (eluent: Pet.Ether:EtOAc=2:1) to afford methyl 2-(3-(2-oxabicyclo[2.2.2]octan-4-yl)-1H-pyrazol-1-yl)propanoate (45 mg, 43%) as a yellow solid. LCMS m/z=265.2 [M+H]⁺; ¹H NMR (400 MHz, DMSO-d₆) δ 7.69 (d, J=2.4 Hz, 1H), 6.13 (d, J=2.4 Hz, 1H), 5.16 (q, J=7.4 Hz, 1H), 3.77 (d, J=1.6 Hz, 2H), 3.72 (dd, J=3.8, 1.8 Hz, 1H), 3.63 (s, 3H), 1.98-1.93 (m, 2H), 1.88 (m, 2H), 1.81 (m, 2H), 1.69-1.65 (m, 2H), 1.61 (d, J=7.2 Hz, 3H).

### Step 6: 2-(3-(2-oxabicyclo[2.2.2]octan-4-yl)-1H-pyrazol-1-yl)propanoic acid

To a solution of methyl 2-(3-(2-oxabicyclo[2.2.2]octan-4-yl)-1H-pyrazol-1-yl)propanoate (55 mg, 0.21 mmol) in a mixture of THF, water and MeOH (4 mL/1 mL/1 mL) was added NaOH (25 mg, 0.62 mmol). The reaction mixture was stirred at room temperature for 4 h then was diluted with water (20 mL), acidified to pH˜2 with 1M HCl and extracted with EtOAc (50 mL×3). The combined organic layers were washed with brine, dried over Na₂SO₄, filtered and concentrated to afford 2-(3-(2-oxabicyclo[2.2.2]octan-4-yl)-1H-pyrazol-1-yl)propanoic acid (34 mg, 65%) as a yellow oil. LCMS m/z=251.2 [M+H]⁻; ¹H NMR (400 MHz, DMSO-d₆) δ 7.65 (d, J=2.4 Hz, 1H), 6.11 (d, J=2.4 Hz, 1H), 4.98 (q, J=7.2 Hz, 1H), 3.77 (t, J=1.4 Hz, 2H), 3.73 (m, 1H), 2.01-1.87 (m, 4H), 1.80 (m, 2H), 1.66 (m, 2H), 1.59 (d, J=7.2 Hz, 3H). Synthesis of N-(3,4-dichlorobenzyl)-N-((tetrahydro-2H-pyran-4-yl)methyl)cyanamide

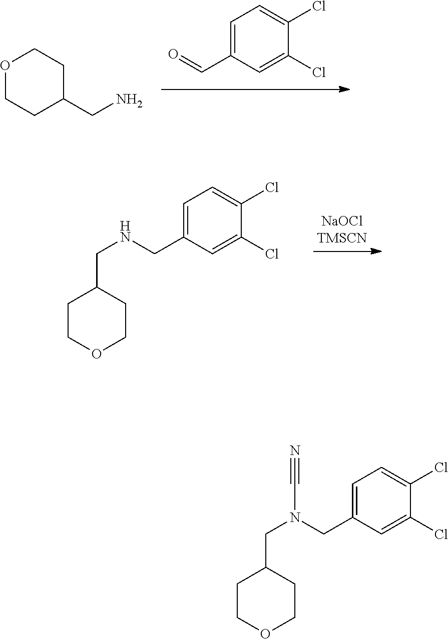 [SMILES: NCC1CCOCC1 ; O=Cc1ccc(Cl)c(Cl)c1 ; Clc1ccc(CNCC2CCOCC2)cc1Cl ; N#CN(Cc1ccc(Cl)c(Cl)c1)CC1CCOCC1]

### Step 1: N-(3,4-dichlorobenzyl)-1-(tetrahydro-2H-pyran-4-yl)methanamine

To a solution of (tetrahydro-2H-pyran-4-yl)methanamine (2.0 g, 17.36 mmol) and 3,4-dichlorobenzaldehyde (3.04 g, 17.4 mmol) in DCE (20 mL) was added sodium triacetoxyborohydride (5.52 g, 126 mmol). The reaction was stirred at room temperature for 5 h then was diluted with water (30 mL) and extracted with DCM (30 mL×2). The aqueous layers were filtered and concentrated to afford N-(3,4-dichlorobenzyl)-1-(tetrahydro-2H-pyran-4-yl)methanamine (3.8 g, 79%) as a yellow oil. LCMS m/z=274.2 [M+H]⁺; ¹HNMR (400 MHz, Methanol-d4) δ 6.85 (d, J=2.7 Hz, 1H), 6.74 (d, J=8.2 Hz, 1H), 6.61-6.53 (m, 1H), 3.21-3.17 (m, 2H), 3.17-3.08 (m, 2H), 2.66-2.55 (m, 2H), 1.96-1.90 (m, 2H), 0.94-0.84 (m, 2H), 0.57-0.43 (m, 3H).

### Step 2: N-(3,4-dichlorobenzyl)-N-((tetrahydro-2H-pyran-4-yl)methyl)cyanamide

To a solution of NaOCl (4% in H₂O, 20.36 g, 10.94 mmol) in MeCN (10 mL) was added TMSCN (960 mg, 7.29 mmol) and the mixture stirred at room temperature for 10 min. N-(3,4-dichlorobenzyl)-1-(tetrahydro-2H-pyran-4-yl)methanamine (1.0 g, 3.65 mmol) was added and stirring continued for 24 h. The mixture was diluted with water (50 mL) and extracted with DCM (20 mL×3). The combined organic layers were washed with brine, dried over Na₂SO₄, filtered and concentrated, The mixture was purified by RP column (eluent: 65% MeCN in H₂O) to afford N-(3,4-dichlorobenzyl)-N-((tetrahydro-2H-pyran-4-yl)methyl)cyanamide (240 mg, 20%) as red oil. LCMS m/z=299.1 [M+H]⁺; ¹H NMR (400 MHz, DMSO-d6) δ 7.74-7.64 (m, 2H), 7.31-7.27 (m, 1H), 4.52-4.47 (m, 2H), 3.93-3.89 (m, 2H), 3.32-3.26 (m, 2H), 3.14-3.09 (m, 2H), 1.97-1.85 (m, 1H), 1.57-1.52 (m, 2H), 1.33-1.27 (in, 2H).

Table 38: The compounds listed in Table 38 were synthesized from according to the procedures outlined for I-219 using the appropriate commercially available reagents and/or intermediates described elsewhere.

TABLE 38

| Example | | | |
|---|---|---|---|
| Number | Compound | ¹HNMR | LCMS |
| I-7 | 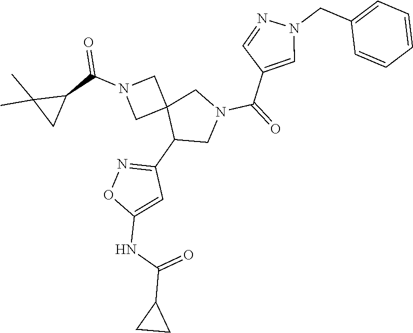 [SMILES: CC1(C)C[C@@H]1C(=O)N1CC2(CN(C(=O)c3cnn(Cc4ccccc4)c3)CC2c2cc(NC(=O)C3CC3)on2)C1] | ¹H NMR (400 MHz, CD₃OD) δ 8.24 − 7.94 (m, 2H), 7.42 − 7.21 (m, 5H), 6.41 − 5.37 (m, 3H), 4.46 − 3.63 (m, 10H), 1.78 − 1.30 (m, 2H), 1.18 − 1.07 (m, 4H), 1.01 − 0.97 (m, 3H), 0.94 − 0.68 (m, 4H). | m/z = 543.5 [M + H]⁺ |
| I-90 | 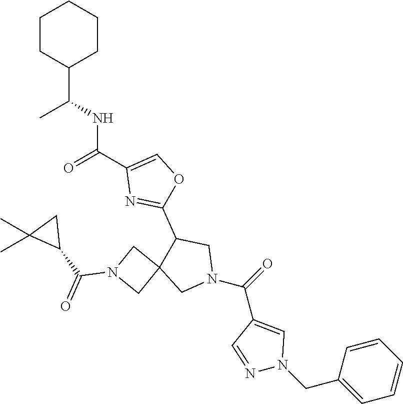 [SMILES: C[C@@H](NC(=O)c1coc(C2CN(C(=O)c3cnn(Cc4ccccc4)c3)CC23CN(C(=O)[C@H]2CC2(C)C)C3)n1)C1CCCCC1] | ¹H NMR (400 MHz, DMSO-d₆) δ 8.63 − 8.54 (m, 1H), 8.39 (d, J = 11.4 Hz, 1H), 7.86 (d, J = 13.4 Hz, 1H), 7.71 (t, J = 11.4 Hz, 1H), 7.41 − 7.23 (m, 5H), 5.37 (s, 2H), 4.39 − 3.74 (m, 10H), 1.64 (d, J = 33.0 Hz, 6H), 1.49 − 1.32 (m, 2H), 1.17 (d, J = 12.8 Hz, 1H), 1.13 − 1.11 (m, 2H), 1.09 (d, J = 6.8 Hz, 3H), 1.03 (dd, J = 13.4, 8.2 Hz, 3H), 0.96 − 0.77 (m, 5H), 0.63 (dd, J = 21.4, 6.4 Hz, 1H). | m/z = 613.3 [M + H]⁺ |
| I-10 | 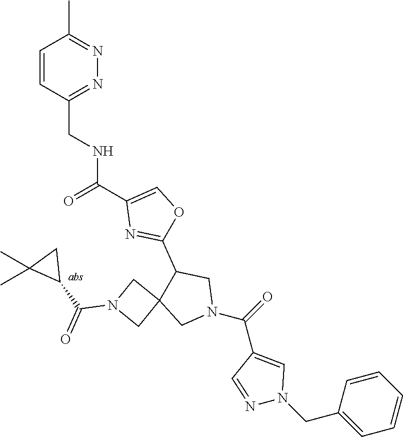 [SMILES: Cc1ccc(CNC(=O)c2coc(C3CN(C(=O)c4cnn(Cc5ccccc5)c4)CC34CN(C(=O)[C@H]3CC3(C)C)C4)n2)nn1] | ¹H NMR (400 MHz, DMSO-d₆) δ 8.91 (s, 1H), 8.67 (d, J = 14.4 Hz, 1H), 8.39 (d, J = 10 Hz, 1H), 7.86 (d, J = 14 Hz, 1H), 7.52 − 7.45 (m, 2H), 7.35 − 7.27 (m, 5H), 5.36 (s, 2H), 4.67 (d, J = 6 Hz, 2H), 4.38 − 4.15 (m, 2H), 4.12 − 3.92 (m, 6H), 3.79 (s, 1H), 2.58 (s, 3H), 1.35 − 1.34 (m, 1H), 1.28 − 1.25 (m, 2H), 1.11 − 1.10 (m, 1H), 1.08 − 1.03 (m, 2H), 0.97 − 0.92 (m, 1H), 0.84 − 0.82 (m, 1H), 0.67 − 0.64 (m, 1H). | m/z = 609.3 [M + H]⁺ |
| I-94 (B) | 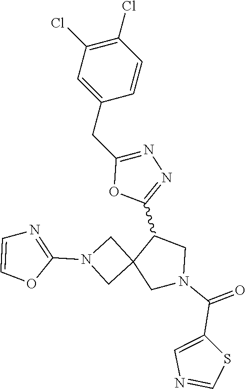 [SMILES: O=C(c1cncs1)N1CC(c2nnc(Cc3ccc(Cl)c(Cl)c3)o2)C2(C1)CN(c1ncco1)C2] | ¹H NMR (400 MHz, Methanol- d4) δ 9.16 (s, 1H), 8.37 (s, 1H), 7.51 (d, J = 7.0 Hz, 1H), 7.43 − 7.36 (m, 2H), 7.19 (t, J = 7.2 Hz, 1H), 6.81 (d, J = 5.2 Hz, 1H), 4.37 − 3.97 (m, 11H) | m/z = 517.1 [M + H]⁺ |
| I-94 (C) | 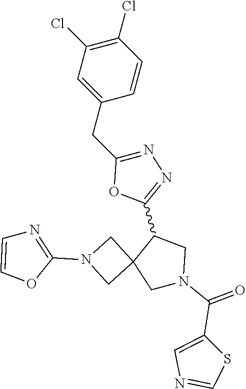 [SMILES: O=C(c1cncs1)N1CC(c2nnc(Cc3ccc(Cl)c(Cl)c3)o2)C2(C1)CN(c1ncco1)C2] | ¹H NMR (400 MHz, Methanol- d4) δ 9.15 (s, 1H), 8.36 (s, 1H), 7.49 (d, J = 6.8 Hz, 1H), 7.39 (d, J = 7.2 Hz, 2H), 7.19 (t, J = 7.2 Hz, 1H), 6.80 (d, J = 5.6 Hz, 1H), 4.34 − 3.96 (m, 11H). | m/z = 517.1 [M + H]⁺ |
| I-102 | 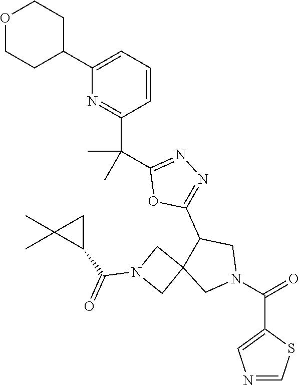 [SMILES: CC(C)(c1cccc(C2CCOCC2)n1)c1nnc(C2CN(C(=O)c3cncs3)CC23CN(C(=O)[C@H]2CC2(C)C)C3)o1] | ¹H NMR (400 MHz, Methanol- d₄) δ 9.16 (s, 1H), 8.43 − 8.30 (m, 1H), 7.75 − 7.60 (m, 1H), 7.21 − 7.14 (m, 1H), 7.12 (d, J = 7.7 Hz, 1H), 4.48 − 4.19 (m, 4H), 4.14 − 3.89 (m, 7H), 3.57 − 3.44 (m, 2H), 2.92 − 2.78 (m, 1H), 1.85 − 1.69 (m, 10H), 1.45 − 1.32 (m, 1H), 1.19 − 1.00 (m, 7H), 0.81 − 0.73 (m, 1H). | m/z = 591.3 [M + H]⁺ |
| I-57 | 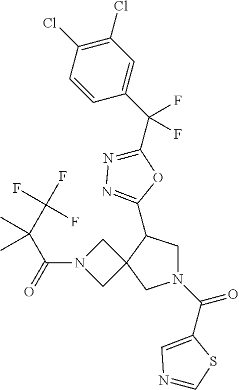 [SMILES: CC(C)(C(=O)N1CC2(CN(C(=O)c3cncs3)CC2c2nnc(C(F)(F)c3ccc(Cl)c(Cl)c3)o2)C1)C(F)(F)F] | ¹H NMR (400 MHz, CD₃OD) δ 9.17 (s, 1H), 8.38 (d, J = 2.8 Hz, 1H), 7.88 (s, 1H), 7.74 (d, J = 8.4 Hz, 1H), 7.66 − 7.58 (m, 1H), 4.70 − 3.96 (m, 9H), 1.37 (s, 6H). | m/z = 624.2 [M + H]⁺ |
| I-84 | 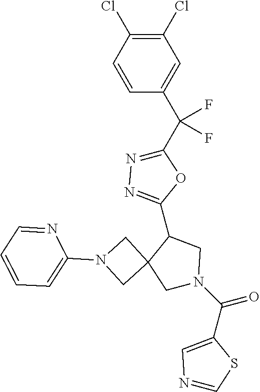 [SMILES: O=C(c1cncs1)N1CC(c2nnc(C(F)(F)c3ccc(Cl)c(Cl)c3)o2)C2(C1)CN(c1ccccn1)C2] | ¹H NMR (400 MHz, DMSO-d₆) δ 9.27 (s, 1H), 8.40 (d, J = 9.2 Hz, 1H), 8.05 (s, 1H), 7.97 (d, J = 7.8 Hz, 1H), 7.78 (d, J = 8.4 Hz, 1H), 7.64 (d, J = 8.0 Hz, 1H), 7.50 (s, 1H), 6.66 (d, J = 6.6 Hz, 1H), 6.34 (d, J = 8.2 Hz, 1H), 4.24 (dd, J = 20.5, 9.3 Hz, 3H), 4.03 (t, J = 11.6 Hz, 3H), 3.91 (t, J = 13.4 Hz, 3H). | m/z = 563.1 [M + H]⁺ |
| I-206 | 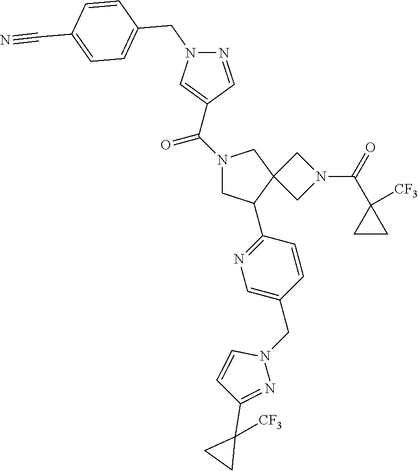 [SMILES: N#Cc1ccc(Cn2cc(C(=O)N3CC(c4ccc(Cn5ccc(C6(C(F)(F)F)CC6)n5)cn4)C4(C3)CN(C(=O)C3(C(F)(F)F)CC3)C4)cn2)cc1] | ¹H NMR (400 MHz, Chloroform-d) δ 8.56 (s, 1H), 8.04 (s, 1H), 7.91 (s, 1H), 7.68 (d, J = 7.6 Hz, 3H), 7.42 − 7.32 (m, 4H), 6.45 (s, 1H), 5.42 (s, 2H), 5.36 (s, 2H), 4.49 − 3.79 (m, 9H), 1.41 − 1.24 (m, 4H), 1.22 − 1.05 (m, 4H). | m/z = 723.0 [M + H]⁺ |
| I-237 | 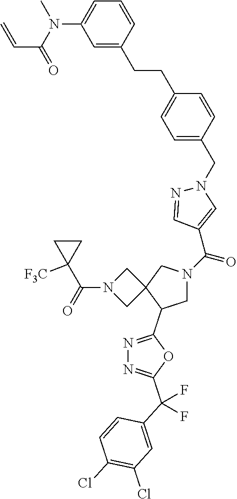 [SMILES: C=CC(=O)N(C)c1cccc(CCc2ccc(Cn3cc(C(=O)N4CC(c5nnc(C(F)(F)c6ccc(Cl)c(Cl)c6)o5)C5(C4)CN(C(=O)C4(C(F)(F)F)CC4)C5)cn3)cc2)c1] | ¹H NMR (400 MHz, Chloroform-d) δ 7.84 − 7.75 (m, 3H), 7.60 (d, J = 8.3 Hz, 1H), 7.48 (d, J = 8.5 Hz, 1H), 7.33 − 7.29 (m, 1H), 7.19 − 7.10 (m, 5H), 6.98 (d, J = 8.1 Hz, 1H), 6.83 (s, 1H), 6.31 (d, J = 16.9 Hz, 1H), 5.99 (s, 1H), 5.47 (d, J = 10.3 Hz, 1H), 5.27 (s, 2H), 4.15 (d, J = 55.3 Hz, 9H), 3.25 (s, 3H), 2.92 (s, 4H), 1.21 (s, 4H). | m/z = 882.4 [M + H]⁺ |
| I-211 | 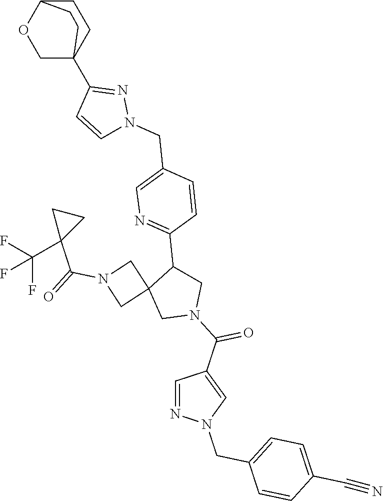 [SMILES: N#Cc1ccc(Cn2cc(C(=O)N3CC(c4ccc(Cn5ccc(C67CCC(CC6)OC7)n5)cn4)C4(C3)CN(C(=O)C3(C(F)(F)F)CC3)C4)cn2)cc1] | ¹H NMR (400 MHz, Methanol- d₄) δ 8.41 (s, 1H), 8.31 (d, J = 10.8 Hz, 1H), 7.97 (d, J = 10.4 Hz, 1H), 7.72 (t, J = 8.6 Hz, 2H), 7.64 − 7.58 (m, 2H), 7.41 (m, 3H), 6.18 (d, J = 2.4 Hz, 1H), 5.48 (d, J = 9.8 Hz, 2H), 5.33 (s, 2H), 4.57 (s, 1H), 4.31 − 3.97 (m, 6H), 3.94 (d, J = 1.8 Hz, 2H), 3.92 − 3.71 (m, 3H), 2.15 − 1.99 (m, 4H), 1.98 − 1.88 (m, 2H), 1.81 − 1.72 (m, 2H), 1.25 − 1.01 (m, 4H). | m/z = 725.3 [M + H]⁺ |
| I-212 | 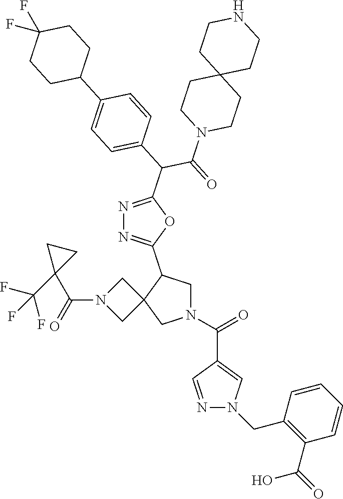 [SMILES: O=C(O)c1ccccc1Cn1cc(C(=O)N2CC(c3nnc(C(C(=O)N4CCC5(CCNCC5)CC4)c4ccc(C5CCC(F)(F)CC5)cc4)o3)C3(C2)CN(C(=O)C2(C(F)(F)F)CC2)C3)cn1] | ¹H NMR (400 MHz, Chloroform-d) δ 8.08 − 7.99 (m, 5H), 7.85 − 7.78 (m, 1H), 7.54 7.50 (m, 1H), 7.43 (d, J = 7.2 Hz, 1H), 7.32 (d, J = 7.4 Hz, 2H), 5.87 − 5.62 (m, 2H), 5.55 (s, 1H), 4.21 − 4.00 (m, 6H), 3.88 − 3.71 (m, 2H), 3.67 − 3.45 (m, 4H), 3.09 (s, 4H), 2.63 − 2.51 (m, 1H), 2.24 2.18 (m, 6H), 1.93 − 1.90 (m, 5H), 1.81 − 1.76 (m, 4H), 1.62 − 1.51 (m, 2H), 1.21 − 1.17 (m, 4H). | m/z = 933.5 [M + H]⁺ |
| I-207 | 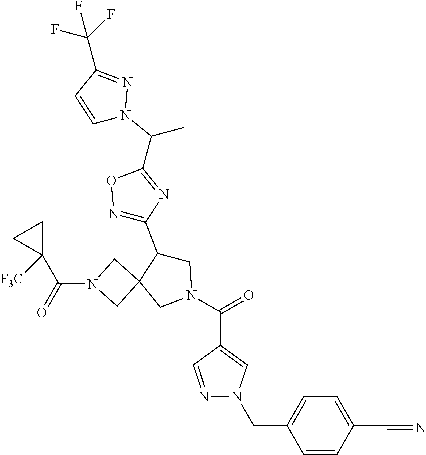 [SMILES: CC(c1nc(C2CN(C(=O)c3cnn(Cc4ccc(C=N)cc4)c3)CC23CN(C(=O)C2(C(F)(F)F)CC2)C3)no1)n1ccc(C(F)(F)F)n1] | ¹H NMR (400 MHz, DMSO-d6) δ 8.42 (d, J = 11.2 Hz, 1H), 8.23 (s, 1H), 7.89 − 7.81 (m, 3H), 7.40 (d, J = 7.9 Hz, 2H), 6.83 (d, J = 2.4 Hz, 1H), 6.29 (d, J = 7.6 Hz, 1H), 5.48 (d, J = 3.8 Hz, 2H), 4.22 3.74 (m, 9H), 1.93 − 1.88 (m, 3H), 1.27 − 1.08 (m, 4H). | m/z = 688.1 [M + H]⁺ |
| I-208 | 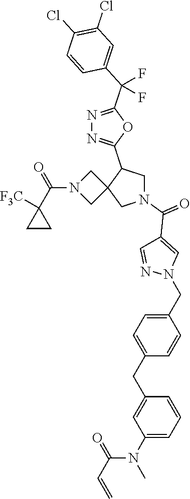 [SMILES: C=CC(=O)N(C)c1cccc(Cc2ccc(Cn3cc(C(=O)N4CC(c5nnc(C(F)(F)c6ccc(Cl)c(Cl)c6)o5)C5(C4)CN(C(=O)C4(C(F)(F)F)CC4)C5)cn3)cc2)c1] | ¹H NMR (400 MHz, CDCl₃) δ 7.87 (s, 1H), 7.78 (d, J = 17.8 Hz, 2H), 7.61 (d, J = 8.4 Hz, 1H), 7.48 (d, J = 8.0 Hz, 1H), 7.32 (t, J = 7.6 Hz, 2H), 7.18 (t, J = 5.8 Hz, 4H), 7.13 (d, J = 7.4 Hz, 1H), 7.04 − 6.95 (m, 2H), 6.36 − 6.28 (m, 1H), 6.05 (s, 1H), 5.49 (d, J = 9.8 Hz, 1H), 5.29 (s, 2H), 4.33 − 3.78 (m, 11H), 3.33 (s, 3H), 1.22 (s, 4H). | m/z = 868.1 [M + H]⁺ |
| I-209 | 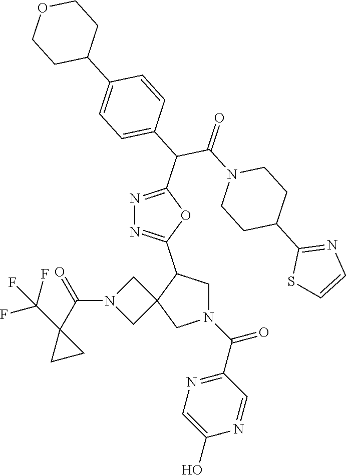 [SMILES: O=C(c1cnc(O)cn1)N1CC(c2nnc(C(C(=O)N3CCC(c4nccs4)CC3)c3ccc(C4CCOCC4)cc3)o2)C2(C1)CN(C(=O)C1(C(F)(F)F)CC1)C2] | ¹H NMR (400 MHz, DMSO-d6) δ 7.95 (s, 2H), 7.72 − 7.54 (m, 2H), 7.36 (d, J = 11.8 Hz, 2H), 7.29 (d, J = 8.0 Hz, 2H), 6.10 − 5.98 (m, 1H), 4.38 (d, J = 12.8 Hz, 1H), 4.20 − 3.85 (m, 12H), 3.42 (d, J = 9.2 Hz, 3H), 3.21 (s, 1H), 2.77 (s, 2H), 2.09 − 1.90 (m, 2H), 1.70 − 1.58 (m, 6H), 1.21 − 1.09 (m, 4H). | m/z = 807.1 [M + H]⁺ |
| I-210 | 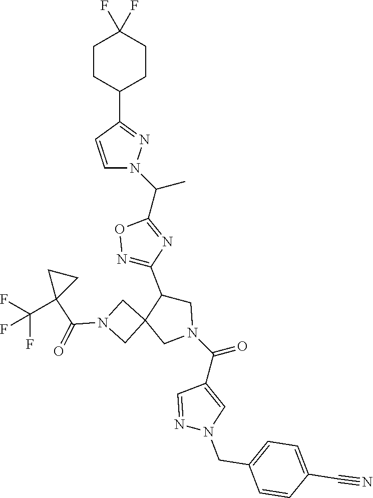 [SMILES: CC(c1nc(C2CN(C(=O)c3cnn(Cc4ccc(C#N)cc4)c3)CC23CN(C(=O)C2(C(F)(F)F)CC2)C3)no1)n1ccc(C2CCC(F)(F)CC2)n1] | ¹H NMR (400 MHz, DMSO-d₆) δ 8.42 (d, J = 11.2 Hz, 1H), 7.95 − 7.76 (m, 4H), 7.47 − 7.33 (m, 2H), 6.19 (d, 1H), 6.08 − 5.95 (m, 1H), 5.55 − 5.43 (m, 2H), 4.45 − 3.70 (m, 9H), 2.77 − 2.66 (m, 1H), 2.04 − 1.81 (m, 9H), 1.66 − 1.54 (m, 2H), 1.28 − 1.01 (m, 4H). | m/z = 738.2 [M + H]⁺ |

### Synthesis of (1-benzyl-1H-pyrazol-4-yl)(8-(4-(benzylamino)phenyl)-2-((S)-2,2-dimethylcyclopropane-1-carbonyl)-2,6-diazaspiro[3.4]octan-6-yl)methanone (I-93)

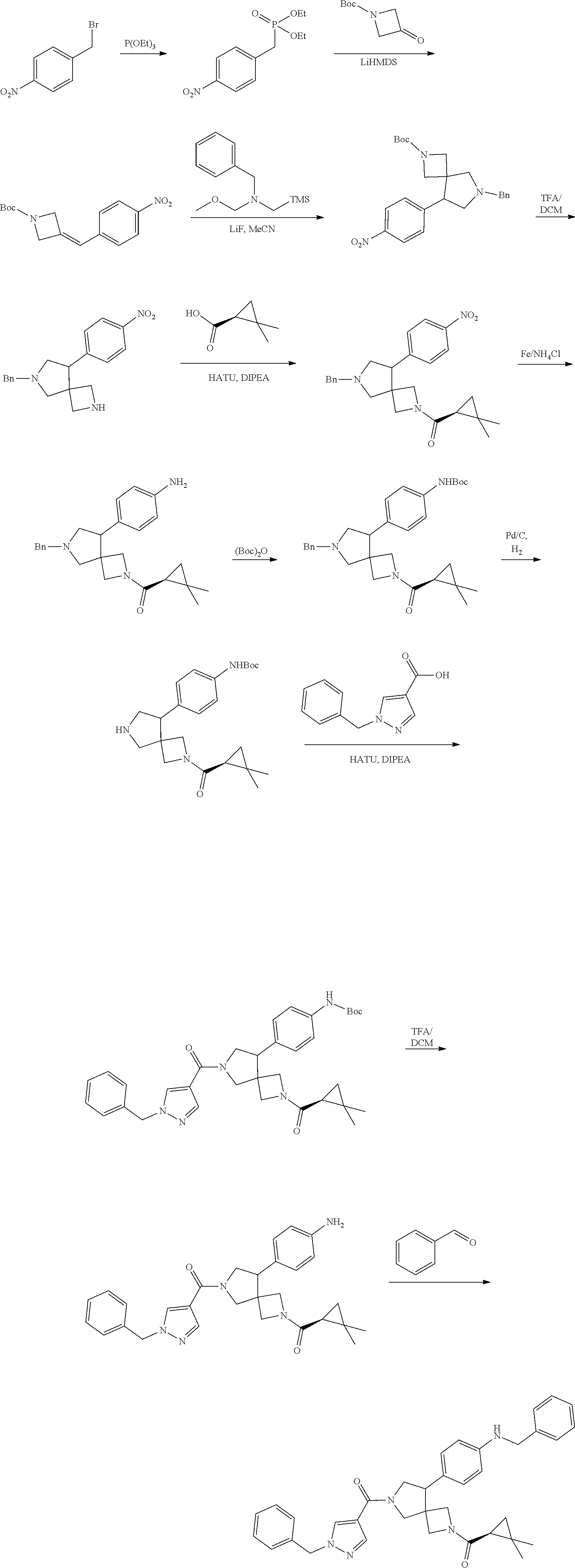

### Step 1: diethyl (4-nitrobenzyl)phosphonate

A solution of 1-(bromomethyl)-4-nitrobenzene (5 g, 23.14 mmol) in P(OEt)₃ (20 mL) was heated at 50° C. overnight. The solvent was removed under vacuum to afford diethyl (4-nitrobenzyl)phosphonate (4 g, 63%) as a red oil which was used directly in the next step. ¹H NMR (400 MHz, DMSO-d₆) δ 8.21-8.14 (m, 2H), 7.56 (m, 2H), 4.02-3.96 (m, 4H), 3.48 (s, 1H), 3.43 (s, 1H), 1.17 (t, J=7.2 Hz, 6H).

### Step 2: tert-butyl 3-(4-nitrobenzylidene)azetidine-1-carboxylate

To a solution of diethyl (4-nitrobenzyl)phosphonate (1 g, 3.66 mol) and tert-butyl 3-oxoazetidine-1-carboxylate (940 mg, 5.49 mol) in anhydrous THF (5 mL) at 0° C. under a N₂ atmosphere was added LiHMDS (1.0 mol in THF) (4.4 mL, 4.39 mmol). The reaction was allowed to warm to room temperature and for 2 h then was diluted with water (100 mL) and extracted with EtOAc (100 mL×2). The combined organic layers were washed with brine, dried over Na₂SO₄, filtered and concentrated. The residue was purified by silica gel column (eluent: DCM:MeOH=80:1) to afford tert-butyl 3-(4-nitrobenzylidene)azetidine-1-carboxylate (900 mg, 84%) as a yellow solid. ¹H NMR (400 MHz, DMSO-d₆) δ 8.19 (d, J=8.8 Hz, 2H), 7.43 (d, J=9.0 Hz, 2H), 6.54 (t, J=2.4 Hz, 1H), 4.87 (s, 2H), 4.62 (s, 2H), 1.42 (s, 9H).

### Step 3: tert-butyl 6-benzyl-8-(4-nitrophenyl)-2,6-diazaspiro[3.4]octane-2-carboxylate

To a solution of tert-butyl 3-(4-nitrobenzylidene)azetidine-1-carboxylate (1 g, 3.45 mmol) in MeCN (10 mL) was added LiF (268 mg, 10.34 mmol) and the reaction heated at 80° C. overnight. The mixture was diluted with water (100 mL) and extracted with EtOAc (100 mL×2). The combined organic layers were washed with brine, dried over Na₂SO₄, filtered and concentrated. The residue obtained was purified by prep-TLC (eluent: Pet.Ether:EtOAc=3:1) to afford tert-butyl 6-benzyl-8-(4-nitrophenyl)-2,6-diazaspiro[3.4]octane-2-carboxylate (180 mg, 12%) as a red oil. LCMS m/z=424.2 [M+H]⁺; ¹H NMR (400 MHz, Methanol-d₄) δ 8.21 (d, J=8.8 Hz, 2H), 7.52 (d, J=8.8 Hz, 2H), 7.39-7.31 (m, 4H), 7.28-7.24 (m, 1H), 3.98-3.87 (m, 2H), 3.72 (d, J=3.2 Hz, 2H), 3.52 (t, J=7.6 Hz, 2H), 3.28 (s, 1H), 3.12 (m, 1H), 3.07-2.97 (m, 2H), 2.76 (m, 1H), 1.33 (s, 9H).

### Step 4: 6-benzyl-8-(4-nitrophenyl)-2,6-diazaspiro[3.4]octane

To a solution of tert-butyl 6-benzyl-8-(4-nitrophenyl)-2,6-diazaspiro[3.4]octane-2-carboxylate (100 mg, 0.24 mmol) in DCM (2 mL) was added TFA (1 mL) and the reaction stirred at room temperature for 1 h. The solvent was removed under vacuum to afford crude 6-benzyl-8-(4-nitrophenyl)-2,6-diazaspiro[3.4]octane (75 mg, 100%) as a red oil which was used directly in the next step. LCMS m/z=324.2 [M+H]⁺.

### Step 5: (6-benzyl-8-(4-nitrophenyl)-2,6-diazaspiro[3.4]octan-2-yl)((S)-2,2-dimethylcyclopropyl)methanone

To a solution of (S)-2,2-dimethylcyclopropane-1-carboxylic acid (32 mg, 0.28 mmol) in DCM (2 mL) was added HATU (132 mg, 0.35 mmol) and DIPEA (90 mg, 0.70 mmol) and the mixture stirred at room temperature for 20 min. 6-benzyl-8-(4-nitrophenyl)-2,6-diazaspiro[3.4]octane (75 mg, 0.23 mmol) was added and stirring continued for 2 h. The mixture was diluted with water (15 mL) and extracted with DCM (15 mL×2). The combined organic layers were washed with brine, dried over Na₂SO₄, filtered and concentrated. The residue obtained was purified by prep-TLC (eluent: DCM:MeOH=20:1) to afford (6-benzyl-8-(4-nitrophenyl)-2,6-diazaspiro[3.4]octan-2-yl)((S)-2,2-dimethylcyclopropyl)methanone (105 mg, 24%) as a red oil. LCMS m/z=420.2 [M+H]⁺; ¹H NMR (400 MHz, DMSO-d₆) δ 8.22 (td, J=8.2, 5.2 Hz, 2H), 7.63-7.56 (m, 2H), 7.39-7.31 (m, 4H), 7.28-7.23 (m, 1H), 4.08-3.90 (m, 2H), 3.87-3.77 (m, 1H), 3.69 (s, 2H), 3.61 (m, 1H), 3.52-3.34 (m, 2H), 2.97 (m, 3H), 1.41-1.38 (m, 1H), 1.25-1.22 (m, 2H), 1.14 (s, 3H), 1.10 (s, 3H).

### Step 6: (8-(4-aminophenyl)-6-benzyl-2,6-diazaspiro[3.4]octan-2-yl)((S)-2,2-dimethylcyclopropyl)methanone

To a suspension of (6-benzyl-8-(4-nitrophenyl)-2,6-diazaspiro[3.4]octan-2-yl)((S)-2,2-dimethylcyclopropyl)methanone (420 mg, 1.00 mmol) and Fe powder (285 mg, 5.01 mmol) in EtOH (6 mL) was added saturated aqueous NH₄Cl (2 mL) and the reaction heated at 80° C. for 1 h. The solvent was removed under vacuum and the residue obtained resuspendened in water (30 mL) and extracted with EtOAc (30 mL×2). The combined organic layers were washed with brine, dried over Na₂SO₄, filtered and concentrated. The residue obtained was purified by prep-TLC (eluent: DCM:MeOH=15:1) to afford (8-(4-aminophenyl)-6-benzyl-2,6-diazaspiro[3.4]octan-2-yl)((S)-2,2-dimethylcyclopropyl)methanone (220 mg, 56%) as a yellow solid. LCMS m/z=390.2 [M+H]⁺.

### Step 7: tert-butyl (4-(6-benzyl-2-((S)-2,2-dimethylcyclopropane-1-carbonyl)-2,6-diazaspiro[3.4]octan-8-yl)phenyl)carbamate

To a solution of (8-(4-aminophenyl)-6-benzyl-2,6-diazaspiro[3.4]octan-2-yl)((S)-2,2-dimethylcyclopropyl)methanone (230 mg, 0.59 mmol) in a mixture of THE and 1.0 M NaOH (0.5 mL/0.5 mL) was added (Boc)₂O (142 mg, 0.65 mmol) and the reaction stirred at room temperature for 2 h. The mixture was diluted with water (30 mL) and extracted with EtOAc (30 mL×2). The combined organic layers were washed with brine, dried over Na₂SO₄, filtered and concentrated to afford tert-butyl (4-(6-benzyl-2-((S)-2,2-dimethylcyclopropane-1-carbonyl)-2,6-diazaspiro[3.4]octan-8-yl)phenyl)carbamate (200 mg, 69%) as a yellow oil. LCMS m/z=490.3 [M+H]⁺; ¹H NMR (400 MHz, DMSO-d6) δ 9.29 (d, J=9.8 Hz, 1H), 7.44-7.30 (m, 6H), 7.25 (m, 1H), 7.13 (q, J=8.4 Hz, 2H), 4.21-3.96 (m, 1H), 3.86-3.60 (m, 4H), 3.47-3.33 (m, 1H), 3.19 (dd, J=9.6, 7.6 Hz, 1H), 3.06-2.79 (m, 3H), 2.63 (m, 1H), 1.47 (s, 9H), 1.13-1.01 (m, 3H), 0.97-0.86 (m, 3H), 0.75 (m, 1H), 0.71 (s, 1H), 0.57 (m, 1H).

### Step 8: tert-butyl (4-(2-((S)-2,2-dimethylcyclopropane-1-carbonyl)-2,6-diazaspiro[3.4]octan-8-yl)phenyl)carbamate

To a solution of tert-butyl (4-(6-benzyl-2-((S)-2,2-dimethylcyclopropane-1-carbonyl)-2,6-diazaspiro[3.4]octan-8-yl)phenyl)carbamate (10 mg, 0.02 mmol) in a mixture of IPrOH and THE (0.5 mL/0.5 mL) was added 10% Pd/C (4 mg) and the reaction stirred under a H₂ atmosphere for 4 h. The catalyst was removed by filtration through Celite and the filtrate concentrated to afford tert-butyl (4-(2-((S)-2,2-dimethylcyclopropane-1-carbonyl)-2,6-diazaspiro[3.4]octan-8-yl)phenyl)carbamate (8 mg, 100%) as a brown oil. LCMS m/z=400.2 [M+H]⁺.

### Step 9: tert-butyl (4-(6-(1-benzyl-1H-pyrazole-4-carbonyl)-2-((S)-2,2-dimethylcyclopropane-1-carbonyl)-2,6-diazaspiro[3.4]octan-8-yl)phenyl)carbamate

To a solution of 1-benzyl-1H-pyrazole-4-carboxylic acid (150 mg, 0.74 mmol) in DCM (2 mL) was added HATU (282 mg, 0.74 mmol) and DIPEA (288 mg, 2.23 mmol) and the mixture stirred at room temperature for 20 mins. Tert-butyl (4-(2-((S)-2,2-dimethylcyclopropane-1-carbonyl)-2,6-diazaspiro[3.4]octan-8-yl)phenyl)carbamate (356 mg, 0.89 mmol) was added and stirring continued for 2 h. The mixture was diluted with water (30 mL) and extracted with DCM (30 mL×2). The combined organic layers were washed with brine, dried over Na₂SO₄, filtered and concentrated. The residue obtained was purified by prep-TLC (eluent: DCM:MeOH=20:1) to afford tert-butyl (4-(6-(1-benzyl-1H-pyrazole-4-carbonyl)-2-((S)-2,2-dimethylcyclopropane-1-carbonyl)-2,6-diazaspiro[3.4]octan-8-yl)phenyl)carbamate (105 mg, 24%) as a yellow solid. LCMS m/z=584.3 [M+H]⁺.

### Step 10: (8-(4-aminophenyl)-2-((S)-2,2-dimethylcyclopropane-1-carbonyl)-2,6-diazaspiro[3.4]octan-6-yl)(1-benzyl-1H-pyrazol-4-yl)methanone

To a solution of tert-butyl (4-(6-(1-benzyl-1H-pyrazole-4-carbonyl)-2-((S)-2,2-dimethylcyclopropane-1-carbonyl)-2,6-diazaspiro[3.4]octan-8-yl)phenyl)carbamate (100 mg, 0.17 mmol) in DCM (2 mL) was added TFA (1 mL) and the reaction stirred at room temperature for 2 h. The solvent was removed under vacuum to afford (8-(4-aminophenyl)-2-((S)-2,2-dimethylcyclopropane-1-carbonyl)-2,6-diazaspiro[3.4]octan-6-yl)(1-benzyl-1H-pyrazol-4-yl)methanone (83 mg, 100%) as a red oil which was used directly in the next step. LCMS m/z=484.3 [M+H]⁺.

### Step 11: (1-benzyl-1H-pyrazol-4-yl)(8-(4-(benzylamino)phenyl)-2-((S)-2,2-dimethylcyclopropane-1-carbonyl)-2,6-diazaspiro[3.4]octan-6-yl)methanone

To a solution of (8-(4-aminophenyl)-2-((S)-2,2-dimethylcyclopropane-1-carbonyl)-2,6-diazaspiro[3.4]octan-6-yl)(1-benzyl-1H-pyrazol-4-yl)methanone (82 mg, 0.17 mmol) in toluene (4 mL) was added TEA (0.5 mL×2), benzaldehyde (36 mg, 0.34 mmol), AcOH (1 mL) and titanium tetraisopropanolate (482 mg, 1.70 mmol). The mixture was heated at reflux overnight then was cooled to room temperature. NaBH₄ (26 mg, 0.68 mmol) was added and stirring continued for 4 h. The mixture was diluted with water (30 mL) and extracted with DCM (30 mL×2). The combined organic layers were washed with brine, dried over Na₂SO₄, filtered and concentrated. The residue obtained was purified by silica gel column (eluent: DCM:MeOH=10:1) and prep-TLC (eluent: DCM:MeOH=20:1) to afford (1-benzyl-1H-pyrazol-4-yl)(8-(4-(benzylamino)phenyl)-2-((S)-2,2-dimethylcyclopropane-1-carbonyl)-2,6-diazaspiro[3.4]octan-6-yl)methanone (25 mg, 26%) as a white solid. LCMS m/z=574.3 [M+H]⁺; ¹H NMR (400 MHz, Methanol-d₄) δ 8.25 (d, J=11.8 Hz, 1H), 7.96 (d, J=10.8 Hz, 1H), 7.37-7.25 (m, 9H), 7.23-7.17 (m, 1H), 7.05 (m, 2H), 6.63 (d, J=7.8 Hz, 2H), 5.38 (d, J=9.8 Hz, 2H), 4.30 (s, 2H), 4.27-3.79 (m, 8H), 3.50-3.34 (m, 1H), 1.31 (m, 1H), 1.17-1.02 (m, 4H), 0.94 (m, 1H), 0.82 (s, 1H).

### Synthesis of (8-(5-(1-(3-(2-oxabicyclo[2.2.2]octan-4-yl)-1H-pyrazol-1-yl)ethyl)-1,2,4-oxadiazol-3-yl)-2-(1-(trifluoromethyl)cyclopropane-1-carbonyl)-2,6-diazaspiro[3.4]octan-6-yl)(1-(4-(trifluoromethyl)benzyl)-1H-pyrazol-4-yl)methanone (I-217)

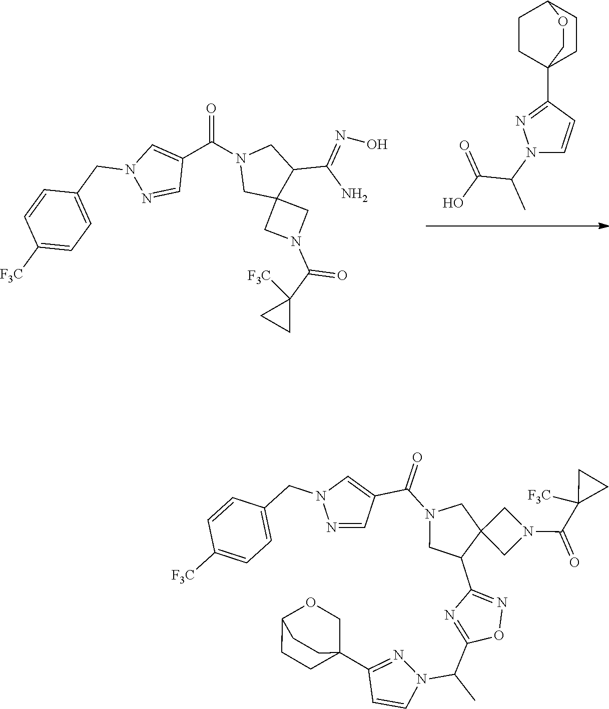 [SMILES: N/C(=N\O)C1CN(C(=O)c2cnn(Cc3ccc(C(F)(F)F)cc3)c2)CC12CN(C(=O)C1(C(F)(F)F)CC1)C2 ; CC(C(=O)O)n1ccc(C23CCC(CC2)OC3)n1 ; CC(c1nc(C2CN(C(=O)c3cnn(Cc4ccc(C(F)(F)F)cc4)c3)CC23CN(C(=O)C2(C(F)(F)F)CC2)C3)no1)n1ccc(C23CCC(CC2)OC3)n1]

To a solution of 2-(3-(2-oxabicyclo[2.2.2]octan-4-yl)-1H-pyrazol-1-yl)propanoic acid (25 mg, 0.1 mmol) in a mixture of DMF (0.5 mL) and dioxane (0.2 mL) in sealed tube was added (Z)—N′-hydroxy-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboximidamide (56 mg, 0.1 mmol) and EDCI (29 mg, 0.15 mmol) and the reaction heated at 60° C. for 3 h, the temperature was increased to 110° C. and stirring continued for 5 h. The mixture was purified by prep-HPLC to afford (8-(5-(1-(3-(2-oxabicyclo[2.2.2]octan-4-yl)-1H-pyrazol-1-yl)ethyl)-1,2,4-oxadiazol-3-yl)-2-(1-(trifluoromethyl)cyclopropane-1-carbonyl)-2,6-diazaspiro[3.4]octan-6-yl)(1-(4-(trifluoromethyl)benzyl)-1H-pyrazol-4-yl)methanone (16.5 mg, 43%) as a white solid. LCMS m/z=773.3 [M+H]⁺; ¹H NMR (400 MHz, DMSO-d₆) δ 8.42 (d, J=10.6 Hz, 1H), 7.89-7.78 (m, 2H), 7.72 (d, J=7.8 Hz, 2H), 7.44 (d, J=7.6 Hz, 2H), 6.18 (s, 1H), 6.04-5.84 (m, 1H), 5.48 (s, 2H), 4.53-4.24 (m, 1H), 4.11 (d, J=22.4 Hz, 3H), 4.02-3.76 (m, 5H), 3.73 (d, J=9.6 Hz, 3H), 1.97-1.75 (m, 9H), 1.62 (t, J=11.8 Hz, 2H), 1.17 (m, 4H).

### Building Blocks

### 2-(4-cyclohexylphenyl)-3-oxo-3-(4-(thiazol-2-yl)piperidin-1-yl)propanehydrazide

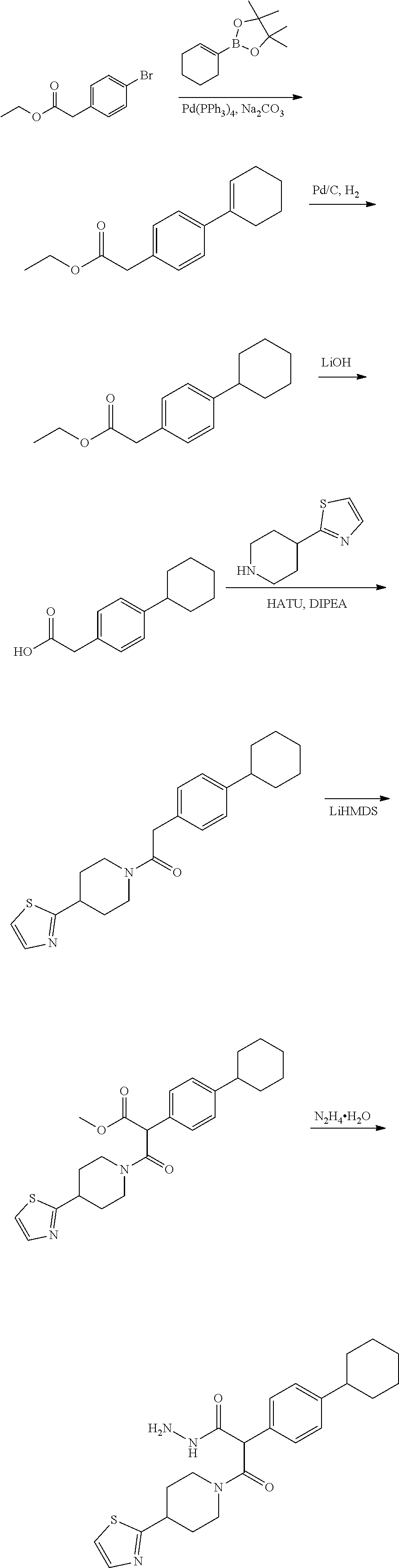

### Step 1: ethyl 2-(2′,3′,4′,5′-tetrahydro-[1,1′-biphenyl]-4-yl)acetate

To a solution of ethyl 2-(4-bromophenyl)acetate (5 g, 0.02 mol) in a mixture of ethylene glycol dimethyl ether (12 mL) and water (6 mL) was added 2-(cyclohex-1-en-1-yl)-4,4,5,5-tetramethyl-1,3,2-dioxaborolane (5.1 g, 0.02 mol), Na₂CO₃ (6.4 g, 0.06 mol) and Pd(PPh₃)₄ (2.4 g, 0.002 mol). The reaction was heated at 100° C. under a N₂ atmosphere overnight then was diluted with water (200 mL) and extracted with EtOAc (200 mL×3). The combined organic layers were dried over Na₂SO₄, filtered and concentrated. The residue was purified by column chromatography on silica gel (eluent: Pet. Ether:EtOAc=80:1, v/v) to afford ethyl 2-(2′,3′,4′,5′-tetrahydro-[1,1′-biphenyl]-4-yl)acetate (1.6 g, 32%) as a colorless oil. ¹H NMR (400 MHz, DMSO-d6) δ 7.34 (d, J=8.0 Hz, 2H), 7.19 (d, J=8.0 Hz, 2H), 6.13 (tt, J=3.8, 1.6 Hz, 1H), 4.07 (q, J=7.0 Hz, 2H), 3.62 (s, 2H), 2.34 (dp, J=6.4, 2.4 Hz, 2H), 2.16 (tt, J=6.0, 3.2 Hz, 2H), 1.71 (m, 2H), 1.60 (m, 2H), 1.18 (t, J=7.0 Hz, 3H).

### Step 2: ethyl 2-(4-cyclohexylphenyl)acetate

To a solution of ethyl 2-(2′,3′,4′,5′-tetrahydro-[1,1′-biphenyl]-4-yl)acetate (1.65 g, 6.76 mmol) in MeOH (10 mL) was added 10% Pd/C (660 mg) and the reaction stirred at 35° C. under H₂ atmosphere overnight. The catalyst was removed by filtration through Celite and the filtrate concentrated to afford the ethyl 2-(4-cyclohexylphenyl)acetate (1.4 g crude, 84%) as a colorless oil, which was used in the next step directly. ¹H NMR (400 MHz, DMSO-d6) δ 7.16 (d, J=1.0 Hz, 4H), 4.10-4.03 (m, 2H), 3.59 (s, 2H), 2.46 (s, 1H), 1.77 (m, 4H), 1.73-1.67 (m, 1H), 1.41-1.31 (m, 4H), 1.25 (m, 1H), 1.20-1.15 (m, 3H).

### Step 3: 2-(4-cyclohexylphenyl)acetic acid

To a solution of ethyl 2-(4-cyclohexylphenyl)acetate (1.55 g, 6.30 mmol) in a mixture of THF, water and MeOH (2.0 mL/0.5 mL/0.5 mL) was added LiOH (793 mg, 18.89 mmol) and the reaction stirred at room temperature for 2 h. The mixture was diluted with water (30 mL) and extracted with EtOAc (30 mL). The aqueous layer was collected, acidified to pH˜2 with 1M HCl and extracted with EtOAc (40 mL×3). The combined organic layers were washed with brine, dried over Na₂SO₄, filtered and concentrated to afford 2-(4-cyclohexylphenyl)acetic acid (1.2 g, 87) as a white solid. ¹H NMR (400 MHz, DMSO-d6) δ 12.26 (s, 1H), 7.15 (s, 4H), 3.50 (s, 2H), 2.45 (d, J=10.0 Hz, 1H), 1.81-1.65 (m, 5H), 1.43-1.21 (m, 5H).

### Step 4: 2-(4-cyclohexylphenyl)-1-(4-(thiazol-2-yl)piperidin-1-yl)ethan-1-one

To a solution of 2-(4-cyclohexylphenyl)acetic acid (270 mg, 1.24 mmol) in DCM (4 mL) was added HATU (471 mg, 1.24 mmol) and DIPEA (481 mg, 3.72 mmol) and the mixture stirred at room temperature for 20 min. 2-(piperidin-4-yl)thiazole (250 mg, 1.49 mmol) was added and stirring continued for 2 h. The mixture was diluted with water (30 mL) and extracted with DCM (30 mL×3). The combined organic layers were washed with brine, dried over Na₂SO₄, filtered and concentrated. The residue obtained was purified by RP-Column (eluent: MeCN:H₂O=60%:40%) to afford 2-(4-cyclohexylphenyl)-1-(4-(thiazol-2-yl)piperidin-1-yl)ethan-1-one (210 mg, 46%) as a yellow solid. LCMS m/z=369.10 [M+H]⁺; ¹H NMR (400 MHz, DMSO-d6) δ 7.70 (d, J=3.2 Hz, 1H), 7.59 (d, J=3.2 Hz, 1H), 7.14 (s, 4H), 4.43 (d, J=13.2 Hz, 1H), 4.01 (d, J=13.6 Hz, 1H), 3.68 (d, J=2.2 Hz, 2H), 3.25 (m, 1H), 3.18-3.10 (m, 1H), 2.74 (m, 1H), 2.47-2.41 (m, 1H), 2.04-1.94 (m, 2H), 1.76 (dd, J=10.2, 5.8 Hz, 4H), 1.69 (d, J=12.6 Hz, 1H), 1.41 (m, 6H), 1.21 (m, 1H).

### Step 5: methyl 2-(4-cyclohexylphenyl)-3-oxo-3-(4-(thiazol-2-yl)piperidin-1-yl)propanoate

To a solution of 2-(4-cyclohexylphenyl)-1-(4-(thiazol-2-yl)piperidin-1-yl)ethan-1-one (150 mg, 0.41 mol) and dimethyl carbonate (81 mg, 0.61 mol) in anhydrous THF (2 mL) at 0° C. under a N₂ atmosphere was added LiHMDS (1.0 mol in THF) (2 mL, 2.04 mmol). The reaction was stirred for 2.5 h then was diluted with water (15 mL) and extracted with EtOAc (15 mL×2). The combined organic layers were washed with brine, dried over Na₂SO₄, filtered and concentrated. The residue was purified by prep-TLC (eluent: Pet.Ether:EtOAc=1:2) to afford methyl 2-(4-cyclohexylphenyl)-3-oxo-3-(4-(thiazol-2-yl)piperidin-1-yl)propanoate (85 mg, 49%) as a white solid. LCMS m/z=427.2 [M+H]⁺; ¹H NMR (400 MHz, DMSO-d6) δ 7.68 (m, 1H), 7.58 (m, 1H), 7.26 (dd, J=8.2, 3.4 Hz, 2H), 7.19 (d, J=8.0 Hz, 2H), 5.30 (d, J=12.0 Hz, 1H), 4.39 (t, J=11.4 Hz, 1H), 3.92 (m, 1H), 3.63 (s, 3H), 3.22 (m, 1H), 2.82-2.68 (m, 1H), 2.01 (m, 2H), 1.83-1.54 (m, 7H), 1.43-1.16 (m, 7H).

### Step 6: 2-(4-cyclohexylphenyl)-3-oxo-3-(4-(thiazol-2-yl)piperidin-1-yl)propanehydrazide

To a solution of methyl 2-(4-cyclohexylphenyl)-3-oxo-3-(4-(thiazol-2-yl)piperidin-1-yl)propanoate (50 mg, 0.12 mmol) in EtOH (1 mL) was added 98% hydrazine hydrate (0.5 mL) and the reaction stirred at room temperature for 1 h. The mixture was diluted with water (10 mL) and extracted with EtOAc (10 mL×2). The combined organic layers were washed with brine, dried over Na₂SO₄, filtered and concentrated to afford 2-(4-cyclohexylphenyl)-3-oxo-3-(4-(thiazol-2-yl)piperidin-1-yl)propanehydrazide (50 mg, 100%) as a white solid. LCMS m/z=427.2 [M+H]⁺; ¹H NMR (400 MHz, DMSO-d6) δ 9.13 (m, 1H), 7.64 (m, 2H), 7.21 (d, J=8.0 Hz, 2H), 7.14 (d, J=8.0 Hz, 2H), 4.81 (d, J=4.6 Hz, 1H), 4.41 (d, J=13.0 Hz, 1H), 4.23 (s, 2H), 3.92 (s, 1H), 3.25 (s, 1H), 3.09 (m, 1H), 2.81-2.69 (m, 1H), 2.46 (s, 1H), 2.01 (s, 1H), 1.87-1.66 (m, 7H), 1.56 (m, 1H), 1.36 (s, 5H), 1.13-1.01 (m, 1H).

### 3-(benzyloxy)-2-(3-cyclohexylphenyl)propanehydrazide

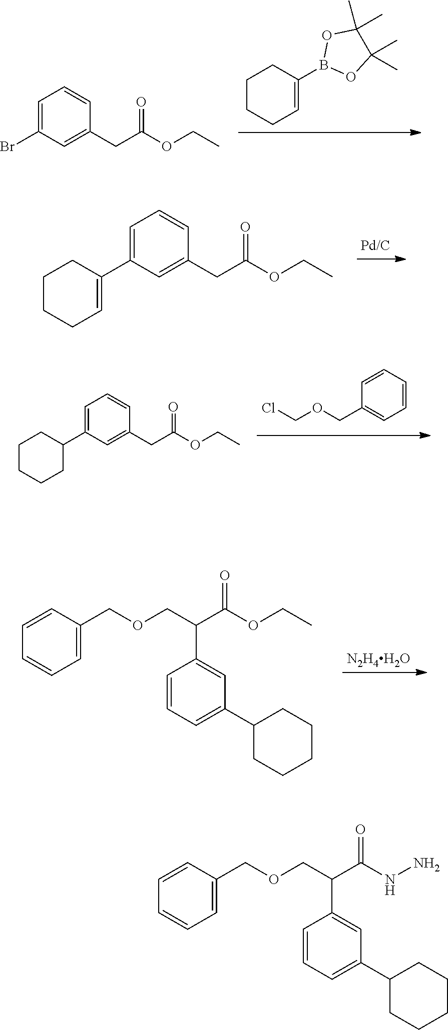

### Step 1: ethyl 2-(2′,3′,4′,5′-tetrahydro-[1,1′-biphenyl]-3-yl)acetate

To a solution of ethyl 2-(3-bromophenyl)acetate (500 mg, 2.1 mmol) in a mixture of dioxane (4 mL) and water (1 mL) under a N₂ atmosphere was added Pd(PPh₃)₄ (119 mg, 0.1 mmol), 2-(cyclohex-1-en-1-yl)-4,4,5,5-tetramethyl-1,3,2-dioxaborolane (472 mg, 2.2 mmol) and K₂CO₃ (570 mg, 4.1 mmol). The reaction was heated at 100° C. for 5 h then was diluted with water (20 mL) and extracted with EtOAc (30 mL×3). The combined organic layers were washed with brine, dried over Na₂SO₄, filtered and concentrated. The residue was purified by column chromatography on silica gel (eluent: Petroleum ether:EtOAc=20:1) to afford ethyl 2-(2′,3′,4′,5′-tetrahydro-[1,1′-biphenyl]-3-yl)acetate (320 mg, 64%) as a colorless oil. ¹H NMR (400 MHz, Chloroform-d) δ 7.31-7.22 (m, 3H), 7.16-7.10 (m, 1H), 6.14-6.08 (m, 1H), 4.14 (q, J=7.1 Hz, 2H), 3.59 (s, 2H), 2.43-2.35 (m, 2H), 2.24-2.15 (m, 2H), 1.81-1.72 (m, 2H), 1.70-1.61 (m, 2H), 1.25 (t, J=7.1 Hz, 3H).

### Step 2: ethyl 2-(3-cyclohexylphenyl)acetate

To a solution of ethyl 2-(2′,3′,4′,5′-tetrahydro-[1,1′-biphenyl]-3-yl)acetate (320 mg, 1.3 mmol) in EtOH (4 mL) was added 10% Pd/C (10 mg) and the reaction stirred under H₂ atmosphere for 2 h. The catalyst was removed by filtration through Celite and the filtrate concentrated to afford ethyl 2-(3-cyclohexylphenyl)acetate (320 mg, quant) as a colorless oil. ¹HNMR (400 MHz, Chloroform-d) δ 7.30-7.25 (m, 1H), 7.18-7.11 (m, 3H), 4.19 (q, J=7.1 Hz, 2H), 3.62 (s, 2H), 2.58-2.47 (m, 1H), 1.96-1.82 (m, 4H), 1.81-1.74 (m, 1H), 1.52-1.22 (m, 8H).

### Step 3: ethyl 3-(benzyloxy)-2-(3-cyclohexylphenyl)propanoate

To a solution of ethyl 2-(3-cyclohexylphenyl)acetate (5 g, 20.3 mmol) and ((chloromethoxy)methyl)benzene (3.4 g, 24.4 mmol) in dry THE (50 mL) at −78° C. was added LiHMDS (1 M in THF, 40.6 mL, 40.6 mmol) dropwise. The reaction was stirred at room temperature for 2 h then was diluted with water (30 mL) and extracted with EtOAc (50 mL×3). The combined organic layers were washed with brine, dried over Na₂SO₄, filtered and concentrated. The mixture was purified by RP-column to afford ethyl 3-(benzyloxy)-2-(3-cyclohexylphenyl)propanoate (4.7 g, 64%) as a colorless oil. ¹H NMR (400 MHz, Chloroform-d) δ 7.36-7.20 (m, 7H), 7.16-7.10 (m, 3H), 4.56 (q, J=12.2 Hz, 2H), 4.26-4.03 (m, 3H), 3.93-3.87 (m, 1H), 3.69-3.63 (m, 1H), 2.52-2.43 (m, 1H), 1.91-1.71 (m, 6H), 1.47-1.32 (m, 4H), 1.23 (t, J=7.1 Hz, 3H).

### Step 4: 3-(benzyloxy)-2-(3-cyclohexylphenyl)propanehydrazide

To a solution of ethyl 3-(benzyloxy)-2-(3-cyclohexylphenyl)propanoate (1 g, 2.7 mmol) in MeOH (3.0 mL) was added hydrazine hydrate (3.0 mL). The reaction was heated at 100° C. for 1 h in a sealed tube then was diluted with water (50 mL) and extracted with EtOAc (30 mL×3). The combined organic layers were washed with brine, dried over Na₂SO₄, filtered and concentrated. The residue was purified by RP-column to afford 3-(benzyloxy)-2-(3-cyclohexylphenyl)propanehydrazide (500 mg, 52%) as a yellow oil. LCMS m/z=352.7 [M+H]⁺.

### 2-amino-5-(2-(thiazol-5-yl)phenoxy)phenol

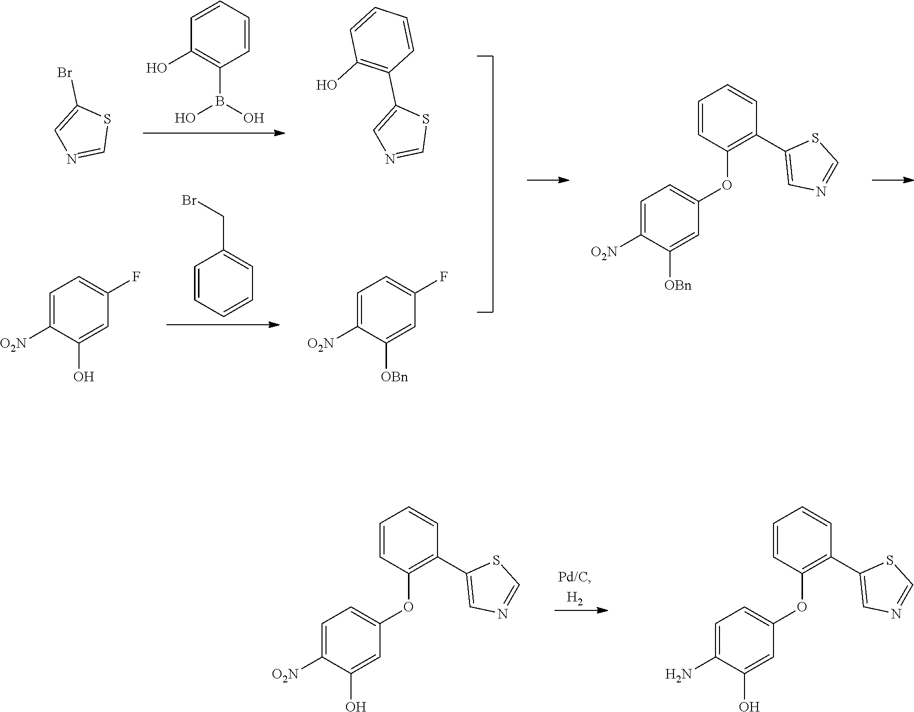

### Step 1: 2-(thiazol-5-yl)phenol

To a solution of 5-bromothiazole (1 g, 6.1 mmol) in a mixture of dioxane and water (9/3 mL) was added (2-hydroxyphenyl)boronic acid (919 mg, 6.7 mmol), Pd(PPh₃)₄ (352 mg, 0.3 mmol) and K₃PO₄ (3.9 g, 18.3 mmol). The reaction was heated at 80° C. under N₂ atmosphere overnight then was diluted with water (30 mL) and extracted with EtOAc (50 mL×3). The combined organic layers were washed with brine, dried over Na₂SO₄, filtered and concentrated to afford 2-(thiazol-5-yl)phenol (964 mg, 88%) as a white solid. LCMS m/z=178.1 [M+H]⁺; ¹H NMR (400 MHz, DMSO-d₆) δ 10.41 (s, 1H), 9.02 (s, 1H), 8.37 (s, 1H), 7.74-7.65 (m, 1H), 7.26-7.14 (m, 1H), 7.01-6.94 (m, 1H), 6.92-6.85 (m, 1H).

### Step 2: 2-(benzyloxy)-4-fluoro-1-nitrobenzene

To a solution of 5-fluoro-2-nitrophenol (1 g, 6.4 mmol) in DMF (10 mL) was added (bromomethyl)benzene (1.2 g, 7.0 mmol) and K₂CO₃ (1.1 g, 7.7 mmol). The mixture was stirred at 60° C. for 3 h then was diluted with water (30 mL) and extracted with EtOAc (50 mL×3). The combined organic layers were washed with brine, dried over Na₂SO₄, filtered and concentrated to afford 2-(benzyloxy)-4-fluoro-1-nitrobenzene (1.2 g, 86%) as a yellow oil. LCMS m/z=247.2 [M+H]⁺; ¹H NMR (400 MHz, CDCl₃) δ 8.00-7.93 (m, 1H), 7.49-7.32 (m, 5H), 6.83 (d, J=10.2 Hz, 1H), 6.74 (d, J=0.8 Hz, 1H), 5.23 (s, 2H).

### Step 3: 5-(2-(3-(benzyloxy)-4-nitrophenoxy)phenyl)thiazole

To a solution of 2-(benzyloxy)-4-fluoro-1-nitrobenzene (1.1 g, 4.7 mmol) in DMF (10 mL) was added 2-(thiazol-5-yl)phenol (832 mg, 4.7 mmol) and K₂CO₃ (650 mg, 4.7 mmol). The reaction was heated at 100° C. overnight then was diluted with water (30 mL) and extracted with EtOAc (30 mL×3). The combined organic layers were washed with brine, dried over Na₂SO₄, filtered and concentrated. The residue obtained was purified by silica gel column (Pet. ether/EtOAc=2/1) to afford 5-(2-(3-(benzyloxy)-4-nitrophenoxy)phenyl)thiazole (1.6 g, 85%) as a yellow oil. LCMS m/z=404.4 [M+H]⁺; ¹H NMR (400 MHz, CDCl₃) δ 8.75 (s, 1H), 8.14 (s, 1H), 7.91 (d, J=9.2 Hz, 1H), 7.80-7.70 (m, 1H), 7.42-7.29 (m, 7H), 7.06 (d, J=7.8 Hz, 1H), 6.62 (d, J=2.2 Hz, 1H), 6.52-6.42 (m, 1H), 5.15 (s, 2H).

### Step 4: 2-nitro-5-(2-(thiazol-5-yl)phenoxy)phenol

To a solution of 5-(2-(3-(benzyloxy)-4-nitrophenoxy)phenyl)thiazole (1.2 g, 3.0 mmol) was added HBr (48% in AcOH, 10 mL) and the reaction heated at 100° C. overnight. The mixture was diluted with water (30 mL) and extracted with EtOAc (30 mL×4). The combined organic layers were washed with brine, dried over Na₂SO₄, filtered and concentrated to afford 2-nitro-5-(2-(thiazol-5-yl)phenoxy)phenol (600 mg, 64%) as a yellow oil. LCMS m/z=314.5 [M+H]⁺; ¹H NMR (400 MHz, CD₃OD) δ 8.98 (s, 1H), 8.24 (s, 1H), 8.12 (d, J=9.4 Hz, 1H), 7.94-7.89 (m, 1H), 7.49-7.41 (m, 2H), 7.30-7.26 (m, 1H), 6.66-6.60 (m, 1H), 6.50 (d, J=2.6 Hz, 1H).

### Step 5: 2-amino-5-(2-(thiazol-5-yl)phenoxy)phenol

To a solution of 2-nitro-5-(2-(thiazol-5-yl)phenoxy)phenol (600 mg, 1.9 mmol) in EtOAc (10 mL) was added 10% Pd/C (300 mg, 1.2 mmol) and the reaction stirred at 45° C. overnight under a hydrogen atmosphere. The catalyst was removed by filtration through Celite and the filtrate concentrated to afford 2-amino-5-(2-(thiazol-5-yl)phenoxy)phenol (475 mg, 87%) as a yellow oil. LCMS m/z=284.6 [M+H]⁺; ¹H NMR (400 MHz, CD₃OD) δ 8.97 (s, 1H), 8.33 (s, 1H), 7.87-7.79 (m, 1H), 7.37-7.30 (m, 1H), 7.24-7.17 (m, 1H), 7.02-6.94 (m, 1H), 6.86 (d, J=8.4 Hz, 1H), 6.49 (d, J=2.6 Hz, 1H), 6.44-6.37 (m, 1H).

### 2-(3-(2-oxabicyclo[2.2.2]octan-4-yl)-1H-pyrazol-1-yl)propanoic acid

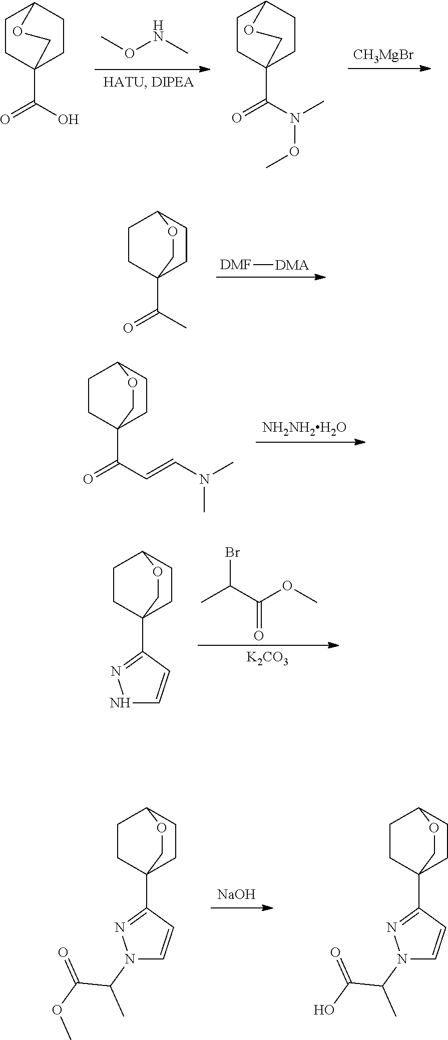

### Step 1: N-methoxy-N-methyl-2-oxabicyclo[2.2.2]octane-4-carboxamide

To a solution of N,O-dimethylhydroxylamine hydrochloride (350 mg, 3.52 mmol) in DCM (8 mL) was added HATU (1.46 g, 3.84 mmol) and the mixture was stirred at room temperature for 30 min. 2-oxabicyclo[2.2.2]octane-4-carboxylic acid (500 mg, 3.2 mmol) and DIPEA (1.65 g, 12.8 mmol) were added and stirring continued for 2 h. The mixture was diluted with water (20 mL) and extracted with DCM (50 mL×3). The combined organic layers were washed with brine, dried over Na₂SO₄, filtered and concentrated to afford N-methoxy-N-methyl-2-oxabicyclo[2.2.2]octane-4-carboxamide (700 mg, 100%) as a yellow oil. LCMS m/z=200.2 [M+H]⁺; ¹H NMR (400 MHz, DMSO-d₆) δ 3.83 (t, J=1.4 Hz, 2H), 3.69 (m, 1H), 3.65 (s, 3H), 3.05 (s, 3H), 2.10-2.01 (m, 2H), 1.93-1.84 (m, 2H), 1.78 (m, 2H), 1.59 (m, 2H).

### Step 2: 1-(2-oxabicyclo[2.2.2]octan-4-yl)ethan-1-one

To a solution of N-methoxy-N-methyl-2-oxabicyclo[2.2.2]octane-4-carboxamide (720 mg, 3.6 mmol) in THF (40 mL) at −78° C. under a N₂ atmosphere was added CH₃MgBr (1 M, 36 mL). The reaction was allowed to warm to room temperature and was stirred overnight. The reaction was diluted with NH₄Cl(aq) (50 mL) and extracted with EtOAc (50 mL×3). The combined organic layers were washed with brine, dried over Na₂SO₄, filtered and concentrated to afford 1-(2-oxabicyclo[2.2.2]octan-4-yl)ethan-1-one (460 mg, 82%) as a yellow oil. ¹H NMR (400 MHz, Chloroform-d) δ 3.92 (s, 2H), 3.82 (m, 1H), 2.12-2.08 (m, 1H), 2.08 (s, 3H), 2.07-2.04 (m, 1H), 1.86 (dd, J=9.0, 6.8 Hz, 4H), 1.69-1.61 (m, 2H).

### Step 3: (E)-1-(2-oxabicyclo[2.2.2]octan-4-yl)-3-(dimethylamino)prop-2-en-1-one

To a solution of 1-(2-oxabicyclo[2.2.2]octan-4-yl)ethan-1-one (160 mg, 1.04 mmol) in DMF (2 mL) in sealed tube was added 1,1-dimethoxy-N,N-dimethylmethanamine (0.5 mL) and the reaction was heated at 100° C. overnight. The mixture was diluted with water (30 mL) and extracted with EtOAc (20 mL×3). The combined organic layers were washed with brine, dried over Na₂SO₄, filtered and concentrated to afford (E)-1-(2-oxabicyclo[2.2.2]octan-4-yl)-3-(dimethylamino)prop-2-en-1-one (76 mg, 35%) as a yellow oil. LCMS m/z=210.2 [M+H]⁺; ¹H NMR (400 MHz, Chloroform-d) δ 7.63 (d, J=12.0 Hz, 1H), 5.06 (d, J=12.2 Hz, 1H), 3.95 (t, J=1.4 Hz, 2H), 3.83 (m, 1H), 3.04 (m, 3H), 2.84 (m, 3H), 2.06-2.02 (m, 2H), 1.95-1.89 (m, 2H), 1.86 (m, 2H), 1.65-1.61 (m, 2H).

### Step 4: 3-(2-oxabicyclo[2.2.2]octan-4-yl)-1H-pyrazole

To a solution of (E)-1-(2-oxabicyclo[2.2.2]octan-4-yl)-3-(dimethylamino)prop-2-en-1-one (215 mg, 1.03 mmol) in EtOH (4 mL) was added hydrazine hydrate (257 mg, 5.14 mmol) and the reaction heated at 80° C. for 3 h. The mixture was concentrated, diluted with water (30 mL) and extracted with EtOAc (20 mL×3). The combined organic layers were washed with brine, dried over Na₂SO₄, filtered and concentrated to afford 3-(2-oxabicyclo[2.2.2]octan-4-yl)-1H-pyrazole (100 mg, 54%) as a yellow oil. LCMS m/z=179.2 [M+H]⁺; ¹H NMR (400 MHz, Chloroform-d) δ 7.49 (d, J=2.2 Hz, 1H), 6.12 (d, J=2.2 Hz, 1H), 3.97 (t, J=1.6 Hz, 2H), 3.90 (m, 1H), 2.17 (m, 2H), 2.06 (m, 2H), 1.98-1.91 (m, 2H), 1.75 (m, 2H).

### Step 5: methyl 2-(3-(2-oxabicyclo[2.2.2]octan-4-yl)-1H-pyrazol-1-yl)propanoate

To a solution of 3-(2-oxabicyclo[2.2.2]octan-4-yl)-1H-pyrazole (70 mg, 0.39 mmol) in DMF (2 mL) was added K₂CO₃ (163 mg, 1.18 mmol) and methyl 2-bromopropanoate (197 mg, 1.18 mmol) and the reaction heated at 70° C. for 3 h. The mixture was concentrated, diluted with water (30 mL) and extracted with EtOAc (20 mL×3). The combined organic layers were washed with brine, dried over Na₂SO₄, filtered and concentrated. The residue was purified by prep-TLC (eluent: Pet.Ether:EtOAc=2:1) to afford methyl 2-(3-(2-oxabicyclo[2.2.2]octan-4-yl)-1H-pyrazol-1-yl)propanoate (45 mg, 43%) as a yellow solid. LCMS m/z=265.2 [M+H]⁺; ¹H NMR (400 MHz, DMSO-d₆) δ 7.69 (d, J=2.4 Hz, 1H), 6.13 (d, J=2.4 Hz, 1H), 5.16 (q, J=7.4 Hz, 1H), 3.77 (d, J=1.6 Hz, 2H), 3.72 (dd, J=3.8, 1.8 Hz, 1H), 3.63 (s, 3H), 1.98-1.93 (m, 2H), 1.88 (m, 2H), 1.81 (m, 2H), 1.69-1.65 (m, 2H), 1.61 (d, J=7.2 Hz, 3H).

### Step 6: 2-(3-(2-oxabicyclo[2.2.2]octan-4-yl)-1H-pyrazol-1-yl)propanoic acid

To a solution of methyl 2-(3-(2-oxabicyclo[2.2.2]octan-4-yl)-1H-pyrazol-1-yl)propanoate (55 mg, 0.21 mmol) in a mixture of THF, water and MeOH (4 mL/1 mL/1 mL) was added NaOH (25 mg, 0.62 mmol). The reaction mixture was stirred at room temperature for 4 h then was diluted with water (20 mL), acidified to pH˜2 with 1M HCl and extracted with EtOAc (50 mL×3). The combined organic layers were washed with brine, dried over Na₂SO₄, filtered and concentrated to afford 2-(3-(2-oxabicyclo[2.2.2]octan-4-yl)-1H-pyrazol-1-yl)propanoic acid (34 mg, 65%) as a yellow oil. LCMS m/z=251.2 [M+H]⁻; ¹H NMR (400 MHz, DMSO-d₆) δ 7.65 (d, J=2.4 Hz, 1H), 6.11 (d, J=2.4 Hz, 1H), 4.98 (q, J=7.2 Hz, 1H), 3.77 (t, J=1.4 Hz, 2H), 3.73 (m, 1H), 2.01-1.87 (m, 4H), 1.80 (m, 2H), 1.66 (m, 2H), 1.59 (d, J=7.2 Hz, 3H).

### N-(3,4-dichlorobenzyl)-N-((tetrahydro-2H-pyran-4-yl)methyl)cyanamide

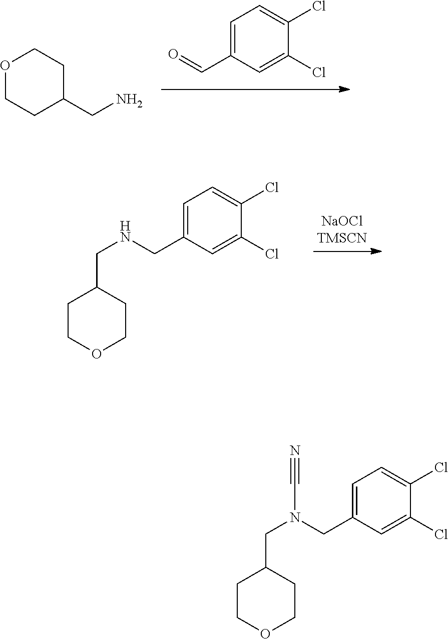 [SMILES: NCC1CCOCC1 ; O=Cc1ccc(Cl)c(Cl)c1 ; Clc1ccc(CNCC2CCOCC2)cc1Cl ; N#CN(Cc1ccc(Cl)c(Cl)c1)CC1CCOCC1]

### Step 1: N-(3,4-dichlorobenzyl)-1-(tetrahydro-2H-pyran-4-yl)methanamine

To a solution of (tetrahydro-2H-pyran-4-yl)methanamine (2.0 g, 17.36 mmol) and 3,4-dichlorobenzaldehyde (3.04 g, 17.4 mmol) in DCE (20 mL) was added sodium triacetoxyborohydride (5.52 g, 126 mmol). The reaction was stirred at room temperature for 5 h then was diluted with water (30 mL) and extracted with DCM (30 mL×2). The aqueous layers were filtered and concentrated to afford N-(3,4-dichlorobenzyl)-1-(tetrahydro-2H-pyran-4-yl)methanamine (3.8 g, 79%) as a yellow oil. LCMS m/z=274.2 [M+H]⁺; ¹HNMR (400 MHz, Methanol-d4) δ 6.85 (d, J=2.7 Hz, 1H), 6.74 (d, J=8.2 Hz, 1H), 6.61-6.53 (m, 1H), 3.21-3.17 (m, 2H), 3.17-3.08 (m, 2H), 2.66-2.55 (m, 2H), 1.96-1.90 (m, 2H), 0.94-0.84 (m, 2H), 0.57-0.43 (m, 3H).

### Step 2: N-(3,4-dichlorobenzyl)-N-((tetrahydro-2H-pyran-4-yl)methyl)cyanamide

To a solution of NaOCl (4% in H₂O, 20.36 g, 10.94 mmol) in MeCN (10 mL) was added TMSCN (960 mg, 7.29 mmol) and the mixture stirred at room temperature for 10 min. N-(3,4-dichlorobenzyl)-1-(tetrahydro-2H-pyran-4-yl)methanamine (1.0 g, 3.65 mmol) was added and stirring continued for 24 h. The mixture was diluted with water (50 mL) and extracted with DCM (20 mL×3). The combined organic layers were washed with brine, dried over Na₂SO₄, filtered and concentrated, The mixture was purified by RP column (eluent: 65% MeCN in H₂O) to afford N-(3,4-dichlorobenzyl)-N-((tetrahydro-2H-pyran-4-yl)methyl)cyanamide (240 mg, 20%) as red oil. LCMS m/z=299.1 [M+H]⁺; ¹HNMR (400 MHz, DMSO-d6) δ 7.74-7.64 (m, 2H), 7.31-7.27 (m, 1H), 4.52-4.47 (m, 2H), 3.93-3.89 (m, 2H), 3.32-3.26 (m, 2H), 3.14-3.09 (m, 2H), 1.97-1.85 (m, 1H), 1.57-1.52 (m, 2H), 1.33-1.27 (m, 2H).

### Synthesis of (5-hydroxy-6-methylpyrazin-2-yl)((8R)-8-(5-(1-(3-(tetrahydro-2H-pyran-4-yl)-1H-pyrazol-1-yl)ethyl)-1,2,4-oxadiazol-3-yl)-2-(1-(trifluoromethyl)cyclopropanecarbonyl)-2,6-diazaspiro[3.4]octan-6-yl)methanone (I-224)

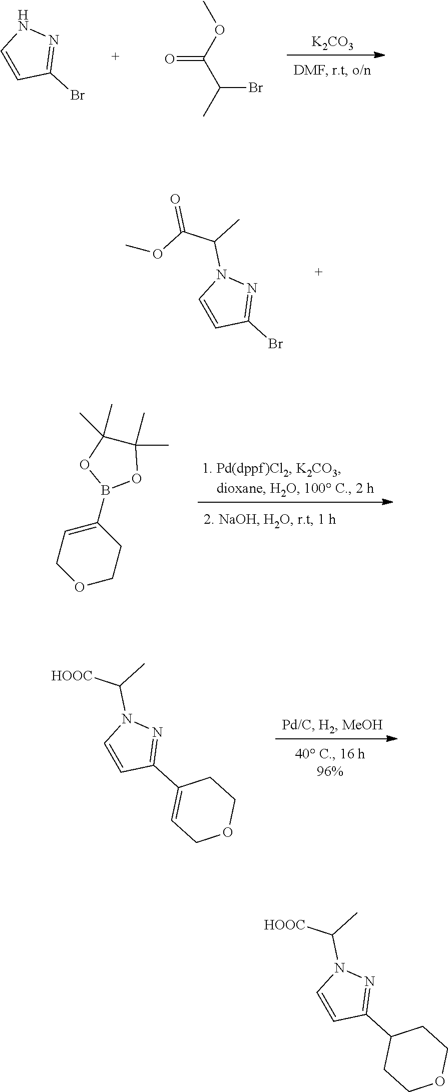

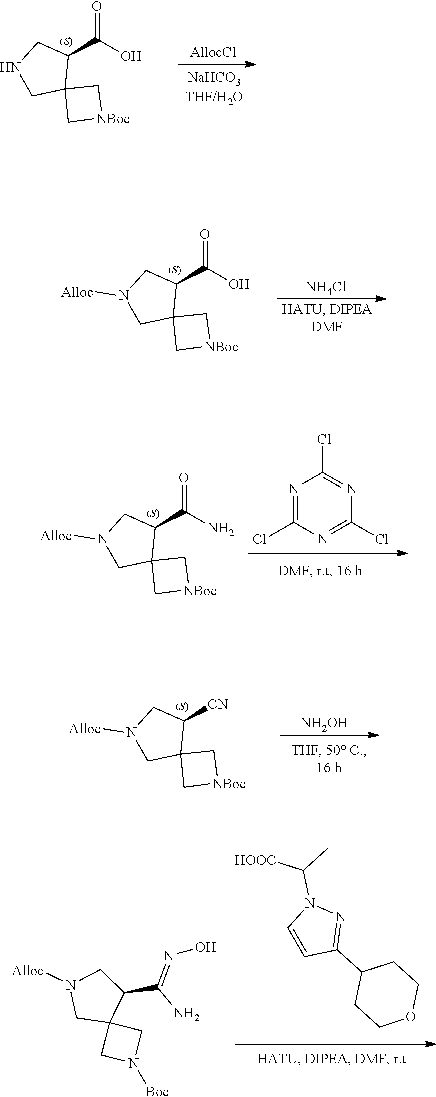

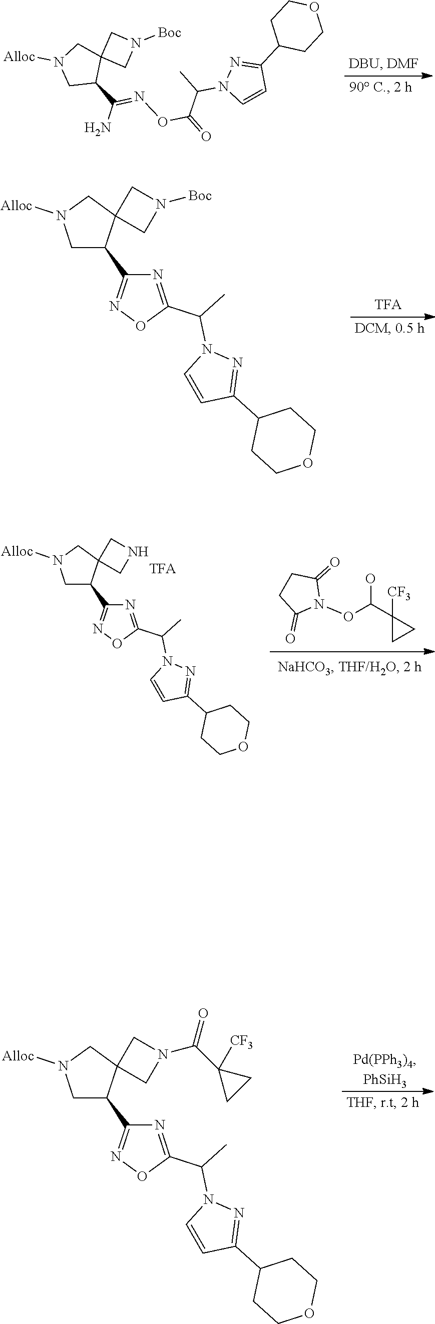

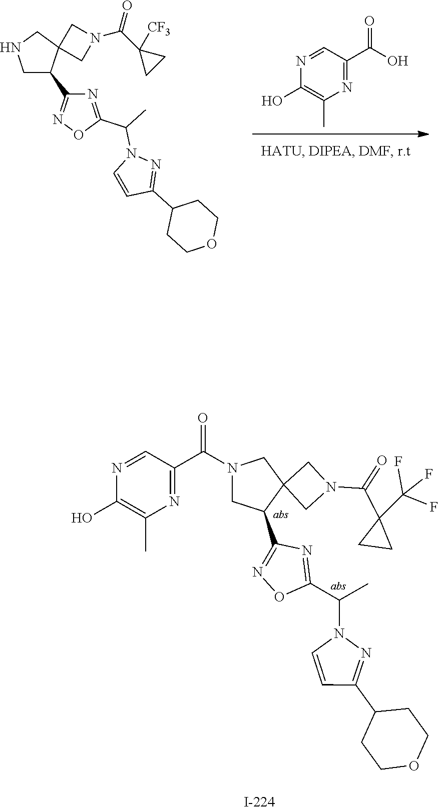

### Methyl 2-(3-bromo-1H-pyrazol-1-yl)propanoate

To a solution of 3-bromo-1H-pyrazole (5.000 g, 34.02 mmol) and K₂CO₃ (9.400 g, 68.04 mmol) in DMF (50 mL) was added methyl 2-bromopropanoate (6.250 g, 37.42 mmol). The resulting mixture was stirred at room temperature for 4 h. The reaction mixture was poured int0 water (75 mL) and extracted with EtOAc (100 mL×3). The combined organic layers were washed with brine (150 mL), dried over Na₂SO₄, and concentrated under reduced pressure to give a residue which was purified by silica gel column chromatography using a 20% EtOAc in hexane gradient to afford methyl 2-(3-bromo-1H-pyrazol-1-yl)propanoate (6.800 g, 83% yield) as a colorless oil. MS: [MH]⁺234.1

### 2-(3-(3,6-Dihydro-2H-pyran-4-yl)-1H-pyrazol-1-yl)propanoic acid

To a solution of methyl 2-(3-bromo-1H-pyrazol-1-yl)propanoate (3.000 g, 12.9 mmol), K₂CO₃ (1.190 g, 8.58 mmol) and 2-(3,6-dihydro-2H-pyran-4-yl)-4,4,5,5-tetramethyl-1,3,2-dioxaborolane (3.260 g, 15.50 mmol) in 1,4-dioxane (30 mL)-H₂O (6 mL) was added Pd(dppf)Cl₂ (0.944 g, 1.29 mmol) N₂ atmosphere. The resulting mixture was stirred at 100° C. under N₂ atmosphere for 4 hours. The reaction mixture was cooled to room temperature, followed by addition of aqueous NaOH solution (10%, 8 mL). The resulting mixture was stirred at room temperature for another 1 hour. The reaction mixture was diluted with water (75 mL) and extracted with TBME (75 mL×2). The aqueous layer was collected, acidified to pH˜3 with hydrochloric acid (2.0 M), and extracted with EtOAc (100 mL×2). The combined organic layers were washed with brine, dried over Na₂SO₄ and concentrated to afford 2-(3-(3,6-dihydro-2H-pyran-4-yl)-1H-pyrazol-1-yl)propanoic acid (2.800 g, 95% yield) as a yellow oil which was used in next step without further purification. MS: [MH]⁺223.0.

### 2-(3-(Tetrahydro-2H-pyran-4-yl)-1H-pyrazol-1-yl)propanoic acid

To a solution of 2-(3-(3,6-dihydro-2H-pyran-4-yl)-1H-pyrazol-1-yl)propanoic acid (2.800 g, 2.25 mmol) in MeOH (50 mL) was added Pd/C (10%, 300 mg). The resulting mixture was stirred at 40° C. under H₂ atmosphere for 16 h. The catalyst was removed through filtration, and the filtrate was concentrated to afford 2-(3-(tetrahydro-2H-pyran-4-yl)-1H-pyrazol-1-yl)propanoic acid (2.700 g, 96% yield) as a yellow oil which was used directly in next step without further purification. MS: [MH]⁺225.0.

### (S)-6-((allyloxy)carbonyl)-2-(tert-butoxycarbonyl)-2,6-diazaspiro[3.4]octane-8-carboxylic acid

To a solution of 2-(tert-butyl) 8-ethyl 2,6-diazaspiro[3.4]octane-2,8-dicarboxylate (3.500 g, 13.70 mmol) and NaHCO₃ (2.300 g, 27.40 mmol) in a mixture of THE (52 mL) and H₂O (52 mL) at 0° C. was added allylchloroformate (2.40 g, 20.2 mmol) dropwise. The resulting mixture was stirred at room temperature for 4 h. The reaction mixture was concentrated under reduce pressure to give a residue which was purified by silica gel column chromatography using a 10% MeOH in DCM gradient to afford (S)-6-((allyloxy)carbonyl)-2-(tert-butoxycarbonyl)-2,6-diazaspiro[3.4]octane-8-carboxylic acid (4.100 g, 88% yield) as a colourless oil. MS: [MH]⁺341.1

### (S)-6-allyl 2-tert-butyl 8-carbamoyl-2,6-diazaspiro[3.4]octane-2,6-dicarboxylate

To a solution of (S)-6-((allyloxy)carbonyl)-2-(tert-butoxycarbonyl)-2,6-diazaspiro[3.4]octane-8-carboxylic acid (2.700 g, 7.94 mmol), NH₄Cl (1.270 g, 23.80 mmol), and DIPEA (3.100 g, 23.80 mmol) in DMF (30 mL) at 0° C. was added HATU (4.520 g, 11.90 mmol). The resulting mixture was stirred at room temperature for 2 hours. The reaction mixture was poured into water (100 mL) and extracted with ethyl acetate (75 mL×3). The combined organic layers were washed with water (100 mL) and then brine (50 mL), dried over anhydrous sodium sulfate, and concentrated under reduced pressure to give a crude residue which was purified by silica gel column chromatography using a 2% MeOH in DCM gradient to afford (S)-6-allyl 2-tert-butyl 8-carbamoyl-2,6-diazaspiro[3.4]octane-2,6-dicarboxylate (1.900 g, 70% yield) as a brown solid. MS: [MH]⁺340.1

### (S)-6-allyl 2-tert-butyl 8-cyano-2,6-diazaspiro[3.4]octane-2,6-dicarboxylate

To a solution of (S)-6-allyl 2-tert-butyl 8-carbamoyl-2,6-diazaspiro[3.4]octane-2,6-dicarboxylate (1.300 g, 3.83 mmol) in DMF (20 mL) was added 2,4,6-trichloro-1,3,5-triazine (1.410 g, 7.66 mmol). The resulting mixture was stirred at room temperature for 2 hours. The reaction mixture was poured into water (50 mL) and extracted with ethyl acetate (35 mL×3). The combined organic layers were washed with water (50 mL) and then brine (30 mL), dried over anhydrous sodium sulfate, and concentrated under reduced pressure to give a crude residue which was purified by silica gel column chromatography using a 3% MeOH in DCM gradient to afford (S)-6-allyl 2-tert-butyl 8-cyano-2,6-diazaspiro[3.4]octane-2,6-dicarboxylate (1.100 g, 81% yield) as a yellow oil. MS: [MH]⁺322.2

### (S,Z)-6-allyl 2-tert-butyl 8-(N′-hydroxycarbamimidoyl)-2,6-diazaspiro[3.4]octane-2,6-dicarboxylate

To a solution of (S)-6-allyl 2-tert-butyl 8-cyano-2,6-diazaspiro[3.4]octane-2,6-dicarboxylate (1.100 g, 3.43 mmol) in EtOH (12 mL) was added NH₂OH H₂O (700 mg, 13.7 mmol). The resulting mixture was stirred at 50° C. for 3 h. The reaction mixture was poured into water (50 mL) and extracted with ethyl acetate (25 mL×3). The combined organic layers were washed with water (35 mL) and then brine (30 mL), dried over anhydrous sodium sulfate, and concentrated under reduced pressure to give a crude residue which was purified by silica gel column chromatography using a 5% MeOH in DCM gradient to afford (S,Z)-6-allyl 2-tert-butyl 8-(N′-hydroxycarbamimidoyl)-2,6-diazaspiro[3.4]octane-2,6-dicarboxylate (1.100 g, 84%) as a white solid. MS: [MH]⁺355.0

### (8R)-6-allyl 2-tert-butyl 8-((Z)—N′-((2-(3-(tetrahydro-2H-pyran-4-yl)-1H-pyrazol-1-yl)propanoyl)oxy)carbamimidoyl)-2,6-diazaspiro[3.4]octane-2,6-dicarboxylate

To a solution of 2-(3-(tetrahydro-2H-pyran-4-yl)-1H-pyrazol-1-yl)propanoic acid (0.380 g, 1.690 mmol), (S,Z)-6-allyl 2-tert-butyl 8-(N′-hydroxycarbamimidoyl)-2,6-diazaspiro[3.4]octane-2,6-dicarboxylate, and DIPEA (0.546 g, 4.23 mmol) in anhydrous DMF (6 mL) at 0° C. was added HATU (0.695 g, 1.83 mmol). The resulting mixture was stirred at room temperature for 1 hour. The reaction mixture was poured into water (30 mL) and extracted with ethyl acetate (15 mL×3). The combined organic layers were washed with water (35 mL) and then brine (30 mL), dried over anhydrous sodium sulfate, and concentrated under reduced pressure to give a crude residue which was purified by silica gel column chromatography using a DCM-EtOAc-Hexane (1:1:2 v/v/v) gradient to afford (8R)-6-allyl 2-tert-butyl 8-((Z)—N′-((2-(3-(tetrahydro-2H-pyran-4-yl)-1H-pyrazol-1-yl)propanoyl)oxy)carbamimidoyl)-2,6-diazaspiro[3.4]octane-2,6-dicarboxylate (0.680 g, 86 yield %) as a yellow oil. MS: [MH]⁺ 561.1

### (8R)-6-allyl 2-tert-butyl 8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2,6-dicarboxylate

To a solution of (8R)-6-allyl 2-tert-butyl 8-((Z)—N′-((2-(3-(tetrahydro-2H-pyran-4-yl)-1H-pyrazol-1-yl)propanoyl)oxy)carbamimidoyl)-2,6-diazaspiro[3.4]octane-2,6-dicarboxylate (0.680 g, 1.21 mmol) in DMF (10 mL) was added DBU (0.220 g, 1.45 mmol), the resulting mixture was stirred at 90° C. for 2 h. The reaction mixture was diluted with water (50 mL) and extracted with EtOAc (50 mL×3). The combined organic layers were washed with brine, dried over Na₂SO₄ and concentrated. The residue was purified by silica gel column chromatography using a 2% MeOH in DCM gradient to afford (8R)-6-allyl 2-tert-butyl 8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2,6-dicarboxylate (0.450 g, 70% yield) as a white solid.

MS: [MH]⁺543.2

### (8R)-allyl 8-(5-(1-(3-(tetrahydro-2H-pyran-4-yl)-1H-pyrazol-1-yl)ethyl)-1,2,4-oxadiazol-3-yl)-2-(1-(trifluoromethyl)cyclopropanecarbonyl)-2,6-diazaspiro[3.4]octane-6-carboxylate

To a solution of (8R)-6-allyl 2-tert-butyl 8-(5-(1-(3-(tetrahydro-2H-pyran-4-yl)-1H-pyrazol-1-yl)ethyl)-1,2,4-oxadiazol-3-yl)-2,6-diazaspiro[3.4]octane-2,6-dicarboxylate (0.450 g, 0.83 mmol) in DCM (8 mL) was added TFA (2 mL). The reaction was stirred for 2 h. The solvent was removed under vacuum to give a residue which was taken up in a mixture of THF (3 mL) and H₂O (3 mL), followed by addition of 2,5-dioxopyrrolidin-1-yl 1-(trifluoromethyl)cyclopropanecarboxylate (0.355 g, 1.40 mmol) and NaHCO₃ (0.392 g, 4.67 mmol). The resulting mixture was stirred at room temperature for 2 h. The reaction mixture was concentrated. The residue was partitioned between EtOAc (50 mL) and H₂O (30 mL). The organic layer was collected, washed with brine (20 mL), dried over Na₂SO₄, and concentrated to give a residue which was purified by silica gel column chromatography using a 2% MeOH in DCM gradient to afford (8R)-allyl 8-(5-(1-(3-(tetrahydro-2H-pyran-4-yl)-1H-pyrazol-1-yl)ethyl)-1,2,4-oxadiazol-3-yl)-2-(1-(trifluoromethyl)cyclopropanecarbonyl)-2,6-diazaspiro[3.4]octane-6-carboxylate (0.460 g, 64% two steps) as a colorless oil. MS: [MH]⁺ 579.1

### ((8R)-8-(5-(1-(3-(tetrahydro-2H-pyran-4-yl)-1H-pyrazol-1-yl)ethyl)-1,2,4-oxadiazol-3-yl)-2,6-diazaspiro[3.4]octan-2-yl)(1-(trifluoromethyl)cyclopropyl)methanone

To a solution of (8R)-allyl 8-(5-(1-(3-(tetrahydro-2H-pyran-4-yl)-1H-pyrazol-1-yl)ethyl)-1,2,4-oxadiazol-3-yl)-2-(1-(trifluoromethyl)cyclopropanecarbonyl)-2,6-diazaspiro[3.4]octane-6-carboxylate (0.460 g, 0.80 mmol) in THF (6 mL) was added Pd(PPh₃)₄ (0.092 g, 0.08 mmol) and phenylsilane (0.431 g, 3.98 mmol); the resulting mixture stirred at 20° C. under nitrogen atmosphere for 2 hours. The reaction mixture was poured into water (20 mL) and extracted with ethyl acetate (15 mL×3). The combined organic layers were washed with water (20 mL) and then brine (10 mL), dried over anhydrous sodium sulfate, and concentrated under reduced pressure to give a crude residue which was purified by silica gel column chromatography using a 5% MeOH in DCM gradient to afford ((8R)-8-(5-(1-(3-(tetrahydro-2H-pyran-4-yl)-1H-pyrazol-1-yl)ethyl)-1,2,4-oxadiazol-3-yl)-2,6-diazaspiro[3.4]octan-2-yl)(1-(trifluoromethyl)cyclopropyl)methanone (0.310 g, 79% yield) as a colorless oil. MS: [MH]⁺495.2

### (5-Hydroxy-6-methylpyrazin-2-yl)(8-(5-(1-(3-(tetrahydro-2H-pyran-4-yl)-1H-pyrazol-1-yl)ethyl)-1,2,4-oxadiazol-3-yl)-2-(1-(trifluoromethyl)cyclopropanecarbonyl)-2,6-diazaspiro[3.4]octan-6-yl)methanone

To a solution of ((8R)-8-(5-(1-(3-(tetrahydro-2H-pyran-4-yl)-1H-pyrazol-1-yl)ethyl)-1,2,4-oxadiazol-3-yl)-2,6-diazaspiro[3.4]octan-2-yl)(1-(trifluoromethyl)cyclopropyl)methanone (0.032 g, 0.06 mmol), 5-hydroxy-6-methylpyrazine-2-carboxylic acid (0.013 g, 0.08 mmol), and DIPEA (0.042 g, 0.33 mmol) in DMF (1 mL) at 20° C. was added HATU (0.030 g, 0.08 mmol); the resulting mixture was stirred at 20° C. for 1 h. The reaction mixture was poured into water (20 mL) and extracted with ethyl acetate (15 mL×3). The combined organic layers were washed with water (15 mL) and then brine (10 mL), dried over anhydrous sodium sulfate, and concentrated under reduced pressure to give a crude residue which was purified by silica gel column chromatography using a 2% MeOH in DCM gradient to afford (5-Hydroxy-6-methylpyrazin-2-yl)(8-(5-(1-(3-(tetrahydro-2H-pyran-4-yl)-1H-pyrazol-1-yl)ethyl)-1,2,4-oxadiazol-3-yl)-2-(1-(trifluoromethyl)cyclopropanecarbonyl)-2,6-diazaspiro[3.4]octan-6-yl)methanone (I-224) (0.016 g, 42%) as a white solid. ¹H NMR (400 MHz, CDCl₃) δ 11.64 (br, 1H), 8.04 (d, J=6.4 Hz, 1H), 7.49-7.48 (m, 1H), 6.17-6.16 (m, 1H), 5.77-5.70 (m, 1H), 4.43-3.91 (m, 10H), 3.64-3.60 (m, 1H), 3.50 (t, J=11.6 Hz, 2H), 2.93-2.86 (m, 1H), 2.47 (d, J=14.0 Hz, 3H), 1.97 (d, J=7.2 Hz, 3H), 1.87-1.74 (m, 4H), 1.19 (br, 4H). MS: [MH]⁺ 631.85

The following 2 compounds were prepared in a manner analogous to the procedures described above for (5-hydroxy-6-methylpyrazin-2-yl)(8-(5-(1-(3-(tetrahydro-2H-pyran-4-yl)-1H-pyrazol-1-yl)ethyl)-1,2,4-oxadiazol-3-yl)-2-(1-(trifluoromethyl)cyclopropanecarbonyl)-2,6-diazaspiro[3.4]octan-6-yl)methanone (I-224)

(5-Hydroxy-6-(trifluoromethyl)pyrazin-2-yl)(8-(5-(1-(3-(tetrahydro-2H-pyran-4-yl)-1H-pyrazol-1-yl)ethyl)-1,2,4-oxadiazol-3-yl)-2-(1-(trifluoromethyl)cyclopropane-1-carbonyl)-2,6-diazaspiro[3.4]octan-6-yl)methanone (I-225) (0.027 g, yield 20%) as a light yellow solid. ¹HNMR (400 MHz, CD₃OD): δ 8.43 (s, 1H), 7.71 (s, 1H), 6.21-6.20 (m, 1H), 5.92-5.84 (m, 1H), 4.61-4.31 (m, 4H), 4.04-3.81 (m, 6H), 3.54-3.48 (m, 2H), 2.90-2.81 (m, 1H), 1.95-1.92 (m, 3H), 1.83-1.67 (m, 4H), 1.35-1.33 (m, 1H), 1.21-1.95 (m, 4H). MS: [MH]⁺685.60

(6-Hydroxy-5-methylpyrazin-2-yl)(8-(5-(1-(3-(tetrahydro-2H-pyran-4-yl)-1H-pyrazol-1-yl)ethyl)-1,2,4-oxadiazol-3-yl)-2-(1-(trifluoromethyl)cyclopropanecarbonyl)-2,6-diazaspiro[3.4]octan-6-yl)methanone (I-226) (0.032 g, 25% yield) as a white solid. ¹HNMR (400 MHz, CD₃OD): δ 8.03 (s, 1H), 7.73-7.71 (m, 1H), 6.22 (s, 1H), 5.93-5.85 (m, 1H), 4.59-4.20 (m, 5H), 4.04-3.84 (m, 6H), 3.55-3.49 (m, 2H), 2.90-2.84 (m, 1H), 2.45 (d, J=3.2 Hz, 3H), 1.94 (t, J=8.0 Hz, 3H), 1.83-1.67 (m, 4H), 1.21 (d, J=6.8 Hz, 4H). MS: [MH]⁺ 631.55.

### Example A1: Caliper Assay

Inhibition of CDK2/Cyclin E1 activity in the presence of compounds of the present disclosure was evaluated using a Caliper LabChip® EZ Reader mobility shift assay. In the assay, activated CDK2/Cyclin E1 catalyzes the phosphorylation of a fluorescently tagged peptide 5-FAM-QSPKKG-CONH2 (PerkinElmer, FL Peptide 18) which induces a difference in capillary electrophoresis mobility. The peptide substrate and product were measured, and the conversion ratio was used to determine the inhibition (as % activity and IC₅₀ values) of CDK2/Cyclin E1. Reactions contained 50 mM HEPES pH 7.5, 10 mM MgCl₂, 1 mM EDTA, 2 mM DTT, 0.01% Brij35, 0.5 mg/mL BSA, 0.1% DMSO, 2.5 nM CDK2/Cyclin E1(14-475), 100 μM ATP, and 1.5 μM fluorescent peptide substrate.

Dose titrations of inhibitors in 100% DMSO were combined with 3.25 nM CDK2/Cyclin E1(14-475) and 130 μM of ATP in reaction buffer. The mixtures were incubated for 30 minutes before the addition of fluorescent peptide substrate to initiate the kinase reaction. The final conditions were 2.5 nM CDK2/Cyclin E1(14-475), 100 μM ATP, and 1.5 μM fluorescent peptide. The reactions were stopped after 100 minutes with the addition of EDTA (6 mM final EDTA concentration). The stopped reactions were analyzed on a Caliper LabChip© EZ Reader II. The conversion ratios were normalized to yield % activity, plotted against compound concentration, and fit to a four-parameter equation to determine the IC₅₀ for each compound.

The results of the Caliper Assay are reported in Table 41, below. Compounds with an IC₅₀ less than or equal to 0.01 μM are designated as “A”. Compounds with an IC₅₀ greater than 0.01 μM and less than or equal to 0.1 μM are designated as “B”. Compounds with an IC₅₀ greater than 0.1 μM and less than or equal to 1.0 μM are designated as “C”. Compounds with an IC₅₀ greater than 1.0 μM and less than or equal to 10.0 μM are designated as “D”. Compounds with an IC₅₀ greater than 10.0 μM are designated as “E”. Compounds with an IC₅₀ greater than 100.0 μM are designated as “F”.

### Example A2: IncuCyte Kuramochi Assay

IncuCyte® assay was used to measure the effect of disclosed compounds on cell proliferation. Fluorescent microscopy images of cells were taken immediately after compound treatment and 72 hours later. Image analysis software was used to obtain cell counts as a function of compound concentration. Kuramochi cells labeled with mApple-H2B were seeded on 384-well assay-ready plates. Plates were placed in an IncuCyte® (Sartorius) and scanned at 0 and 72 hours. IncuCyte® software was used to count the number of fluorescent nuclei in each well. The fold change in cell count from 0 to 72 hours in wells treated with increasing compounds concentrations (10 pts, ½ log dilution, 20 μM top concentration) was normalized to DMSO control wells. The normalized cell counts were fit with dose response curves and a G150 was calculated.

The results of the Kuramochi assay are reported in Table 41, below. Compounds with an IC₅₀ less than or equal to 0.5 μM are designated as “A”. Compounds with an IC₅₀ greater than 0.5 μM and less than or equal to 5.0 μM are designated as “B”. Compounds with an IC₅₀ greater than 5.0 μM and less than or equal to 10.0 μM are designated as “C”. Compounds with an IC₅₀ greater than 10.0 μM are designated as “D”.

### Example A3: ADPGLO (CDK2/E1-37 C)

Inhibition of CDK2/Cyclin E1 activity by the presence of small molecules was evaluated using ADP-Glo Luminescent Kinase Assay (Promega). Activated CDK2/Cyclin E1 was incubated with its substrate Histone H1 (SignalChem H10-54N) in the kinase reaction buffer (100 μM ATP, 50 mM HEPES pH 7.5, 10 mM MgCl2, 1 mM EDTA, 2 mM DTT, 0.01% Brij35, 0.5 mg/mL BSA). Luminescence was recorded with an Envision plate reader (PerkinElmer).

Dose titrations of inhibitors in 100% DMSO were combined with 0.36 nM CDK2/Cyclin E1 in reaction buffer. The mixtures were incubated for 60 minutes at 37° C. before the addition of ATP and Histone H1 substrate to initiate the kinase reaction. The final conditions were 0.18 nM CDK2/Cyclin E1, 100 μM ATP, and 1.5 μM Histone H1. The reactions were incubated at 37° C. for 90 minutes before being stopped with the addition of ADP-Glo reagent. This mixture was incubated at room temperature for 60 minutes before Kinase Detection Solution is added to generate luminescence. The stopped reactions were analyzed on an Envision plate reader. The conversion ratios were normalized to yield % activity, plotted against compound concentration, and fit to a four-parameter equation to determine the IC₅₀ for each compound.

The results of the ADPGLO assay are reported in Table 41 below. Compounds with an IC₅₀ less than or equal to 0.5 μM are designated as “A”. Compounds with an IC₅₀ greater than 0.5 μM and less than or equal to 5.0 μM are designated as “B”. Compounds with an IC₅₀ greater than 5.0 μM and less than or equal to 10.0 μM are designated as “C”. Compounds with an IC₅₀ greater than 10.0 μM are designated as “D”. Compounds with an IC₅₀ greater than 100.0 μM are designated as “E”.

### Table 41. Assay Results

Entries noted in Table 41 as either “first isomer,” “second isomer,” “third isomer,” etc., show the assay data for an isolated stereoisomer or mixture of stereoisomers of said compound, wherein the specific stereochemistry is believed to be one of the stereoisomers designated as “A”, “B,” “C,” “D,” or “E,” in Table 8.

| | | IncuCyte - | ADPGLO |
|---|---|---|---|
| | Caliper Assay | Kuramochi | (CDK2/E1- |
| | (CDK2/CCNE1): | RB-WT: | 37C): IC50 |
| Cmpd # | IC50 (μM) | IC50 (μM) | (μM) |
| I-125 | F | D | E |
| I-126 | C | D | D |
| I-127 | C | D | C |
| I-128 | C | D | D |
| I-129 | F | D | D |
| I-130 | B | D | B |
| I-131 | A | D | B |
| I-132 | D | D | D |
| I-133 | A | B | A |
| I-134 | C | D | B |
| I-135 | F | D | E |
| I-136 | F | D | E |
| I-137 | D | D | D |
| I-138 | E | D | E |
| I-139 | F | D | E |
| I-140 | E | D | D |
| I-141 | F | D | D |
| I-142 | F | D | D |
| I-143 | F | D | D |
| I-144 | D | D | |
| I-145 | E | D | |
| I-146 | F | D | |
| I-147 | F | D | |
| I-148 | E | D | |
| I-149 | E | D | |
| I-150 | E | D | |
| I-151 1st | E | D | |
| I-151 2nd | D | D | |
| I-152 | E | D | |
| I-153 | E | D | |
| I-154 | E | D | |
| I-155 | E | D | |
| I-156 | E | D | |
| I-157 | D | D | |
| I-158 | E | D | |
| I-159 | E | D | |
| I-160 | E | D | |
| I-161 | E | C | |
| I-162 | E | D | |
| I-163 | E | D | |
| I-164 | E | D | |
| I-165 | D | D | |
| I-166 | E | D | |
| I-167 | E | D | |
| I-168 | D | D | |
| I-169 1st | E | C | |
| I-169 2nd | E | D | |
| I-170 | E | D | |
| I-171 | E | D | |
| I-172 1st | E | | |
| I-172 2nd | F | D | D |
| I-173 | E | D | |
| I-174 | D | D | |
| I-175 | E | D | |
| I-176 | D | D | |
| I-177 | E | D | |
| I-178 | E | D | |
| I-179 | E | C | |
| I-180 | E | D | |
| I-181 | E | D | |
| I-182 | E | D | |
| I-183 | E | D | |
| I-184 | E | D | |
| I-185 | E | C | |
| I-186 | E | D | |
| I-187 | E | D | |
| I-188 | D | D | |
| I-189 | D | D | |
| I-190 | E | D | |
| I-191 | D | D | |
| I-192 | D | C | |
| I-193 | D | D | |
| I-194 | C | D | |
| I-195 | D | D | |
| I-196 | E | D | |
| I-197 | E | D | |
| I-198 | B | B | |
| I-199 | E | C | |
| I-200 | E | D | |
| I-201 | C | C | |
| I-202 | E | D | |
| I-203 | E | D | |
| I-204 | E | D | |
| I-205 | E | C | |
| I-62 | B | C | |
| I-61 | B | D | |
| I-60 | E | D | |
| I-59 | E | D | |
| I-58 1st | D | | |
| I-58 2nd | D | D | |
| I-57 | C | D | |
| I-56 | D | D | |
| I-55 | C | D | |
| I-54 | E | D | |
| I-53 | E | D | |
| I-52 | C | D | |
| I-51 | E | D | |
| I-50 | D | D | |
| I-48 | C | D | |
| I-49 | E | D | |
| I-47 | E | D | |
| I-46 | E | D | |
| I-45 1st | C | D | |
| I-45 2nd | C | D | D |
| [symbol] | | | |
| I-44 | D | D | |
| I-43 | D | D | |
| I-42 | D | D | |
| I-41 | E | D | |
| I-40 | C | D | |
| I-100 | C | C | |
| I-99 | E | D | |
| I-98 | E | D | |
| I-97 | C | D | |
| I-38 1st | E | D | |
| I-38 2nd | D | D | |
| I-38 3rd | E | D | |
| I-39 | C | D | |
| I-37 | D | D | |
| I-104 | C | D | |
| I-103 | E | D | |
| I-102 | B | D | |
| I-96 | B | D | |
| I-95 | B | C | |
| I-83 | C | D | |
| I-105 | E | D | |
| I-81 | D | B | |
| I-82 | C | D | |
| I-79 | D | D | |
| I-80 | E | D | |
| I-77 | D | D | |
| I-78 | C | D | |
| I-76 | C | D | |
| I-67 | D | D | |
| I-68 | C | D | |
| I-69 | C | D | |
| I-70 | D | D | |
| I-71 | D | D | |
| I-72 | D | D | |
| I-73 | C | D | |
| I-74 | D | D | |
| I-75 | C | D | |
| I-33 | D | D | |
| I-34 | D | D | |
| I-35 | C | D | |
| I-36 1st | B | D | |
| I-36 2nd | D | D | D |
| I-36 3rd | B | D | A |
| I-36 4th | B | D | |
| I-36 5th | A | D | |
| I-63 | D | D | |
| I-64 | B | D | |
| I-65 | D | D | |
| I-66 | C | D | |
| I-30 | D | D | |
| I-31 | C | D | |
| I-32 | C | D | B |
| I-28 | C | D | |
| I-29 | E | D | |
| I-27 | C | D | |
| I-26 | D | D | |
| I-25 | C | D | |
| I-94 1st | E | D | |
| I-94 2nd | E | D | |
| I-94 3rd | E | D | |
| I-24 | C | C | |
| I-23 | C | D | |
| I-21 | C | D | |
| I-22 | D | D | |
| I-15 | B | C | B |
| I-16 | D | D | |
| I-17 | C | D | |
| I-18 | C | D | |
| I-19 1st | C | D | |
| I-19 2nd | D | D | |
| I-19 3rd | C | D | |
| I-101 | E | D | |
| I-20 | D | D | |
| I-12 | D | D | |
| I-13 | D | D | |
| I-14 | D | D | |
| I-93 | D | C | |
| I-92 | B | C | |
| I-91 | C | D | |
| I-10 | E | D | |
| I-11 | D | A | |
| I-90 | B | C | |
| I-89 | C | C | |
| I-9 | D | D | |
| I-88 | C | D | |
| I-87 | D | C | |
| I-86 | E | D | |
| I-8 | C | C | |
| I-7 | C | C | |
| I-85 | D | D | |
| I-6 | D | C | |
| I-4 | D | D | |
| I-5 | D | D | |
| I-3 | D | D | |
| I-2 | C | C | |
| I-2 | E | C | |
| I-84 | D | D | |
| I-1 | C | C | |
| I-106 | | C | D |
| I-107 | | B | A |
| I-108 | | C | B |
| I-109 | | C | A |
| I-110 | | D | C |
| I-111 | | C | B |
| I-112 | | D | D |
| I-113 | | B | B |
| I-114 | | D | B |
| I-115 | | C | B |
| I-116 | | D | D |
| I-117 | | D | B |
| I-118 | | D | B |
| I-119 | | D | B |
| I-120 | | D | D |
| I-121 | | D | B |
| I-122 | | D | C |
| I-123 | | D | A |
| I-124 | | D | B |
| I-207 | | D | D |
| I-208 | | D | D |
| I-209 | | A | D |
| I-210 | | A | B |
| I-213 | | C | D |
| I-214 | | D | B |
| I-215 | | C | B |
| I-216 | | B | A |
| I-217 | | C | B |
| I-218 | | D | D |
| I-219 | | D | D |
| I-220 | | D | B |
| I-221 | | C | B |
| I-222 | | D | D |
| I-223 | | C | D |
| I-224 | | D | D |
| I-225 | | D | C |
| I-226 | | D | D |

[symbol]

indicates data missing or illegible when filed

1. A compound of Formula I: 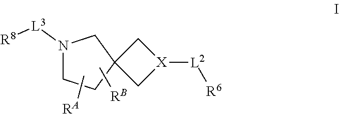
or a pharmaceutically acceptable salt thereof, wherein:
X is N or CR^{B};
R^{A} is 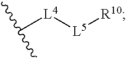
each R^{B} is independently a hydrogen, an optionally substituted C₁₋₆ aliphatic group, or a halogen;
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —C(R)₂—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy¹ group, or Cy¹;
L³ is a covalent bond or a saturated or unsaturated, straight or branched, optionally substituted bivalent C₁₋₄ hydrocarbon chain, wherein 0-4 methylene units of L³ are independently replaced by —O—, —NR—, —S—, —OC(O)—, —C(O)O—, —C(O)—, —S(O)—, —S(O)₂—, —C(S)—, —C(R)₂—, —NRS(O)₂—, —S(O)₂NR—, —NRC(O)—, —C(O)NR—, —OC(O)NR—, —NRC(O)O—, —NRC(O)NR—, or -Cy²-;
L⁴ is optionally substituted phenylene, an optionally substituted bivalent 5-6 membered monocyclic heteroarylene ring (having 1-4 heteroatoms independently selected from nitrogen, oxygen, and sulfur), or an optionally substituted bivalent 8-10 membered bicyclic heteroarylene ring (having 1-5 heteroatoms independently selected from nitrogen, oxygen, and sulfur);
L⁵ is a covalent bond or a saturated or unsaturated, straight or branched, optionally substituted bivalent C₁₋₄ hydrocarbon chain, wherein 0-2 methylene units of L⁵ are independently replaced by —O—, —NR—, —S—, —C(R)₂—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, -Cy²-, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted saturated or unsaturated C₁₋₆ aliphatic group, an optionally substituted C₁₋₆ aliphatic-Cy¹ group, or Cy¹;
R¹⁰ is hydrogen or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-3 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each Cy¹ is independently an optionally substituted and cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur);
each -Cy²- is independently an optionally substituted and bivalent cyclic group selected from a 3-8 membered saturated or partially unsaturated monocyclic carbocyclene, phenylene, a 3-8 membered saturated or partially unsaturated monocyclic heterocyclene ring (having 1-2 heteroatoms independently selected from nitrogen, oxygen, and sulfur), and a 5-6 membered monocyclic heteroarylene ring (having 1-4 heteroatoms independently selected from nitrogen, oxygen, and sulfur); and
each R is independently hydrogen, halogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), an optionally substituted 5-6 membered heteroaryl ring (having 1-4 heteroatoms independently selected from nitrogen, oxygen, and sulfur), two R groups on the same nitrogen atom or carbon atom are taken together with the nitrogen atom or carbon atom to form an optionally substituted 3-7 membered saturated, partially unsaturated, or heteroaryl ring (having 0-3 heteroatoms independently selected from nitrogen, oxygen, and sulfur), or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur).

2. The compound of claim 1, wherein X is N.

3. The compound of claim 1, wherein R^{B} is hydrogen.

4-26. (canceled)

27. The compound of claim 1, wherein the compound of Formula I is a compound of Formula II: 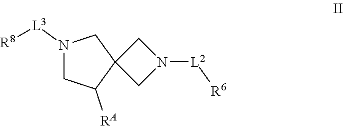
or a pharmaceutically acceptable salt thereof.

28. (canceled)

29. The compound of claim 1, wherein the compound of Formula I is a compound of Formula IIIc, IIId, IIIe, IIIf, IIIg, IIIh, IIIi, IIIj, IIIk IIIl, IIIm, IIIn, IIIo, IIIp, IIIq, IIIr, IIIs, IIIt, IIIu, IIIv, IIIw, IIIx, IIIy, or IIIz: 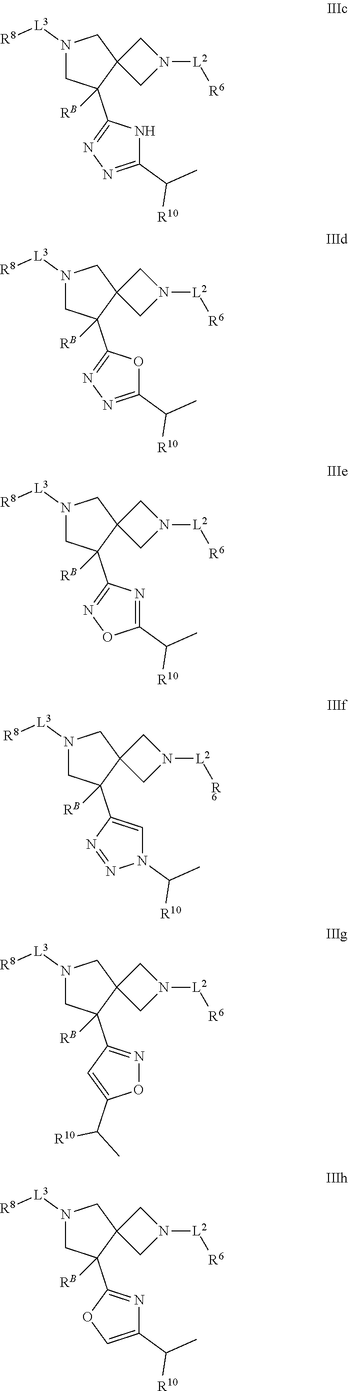 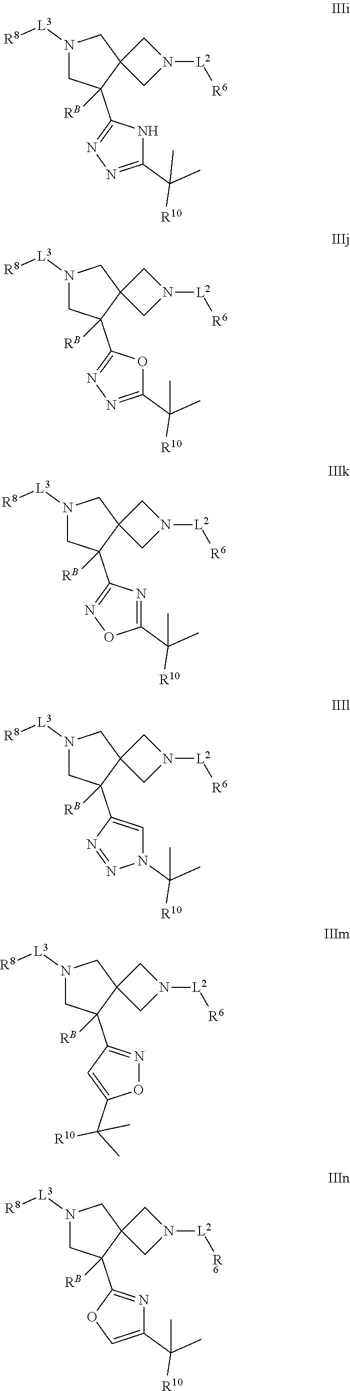 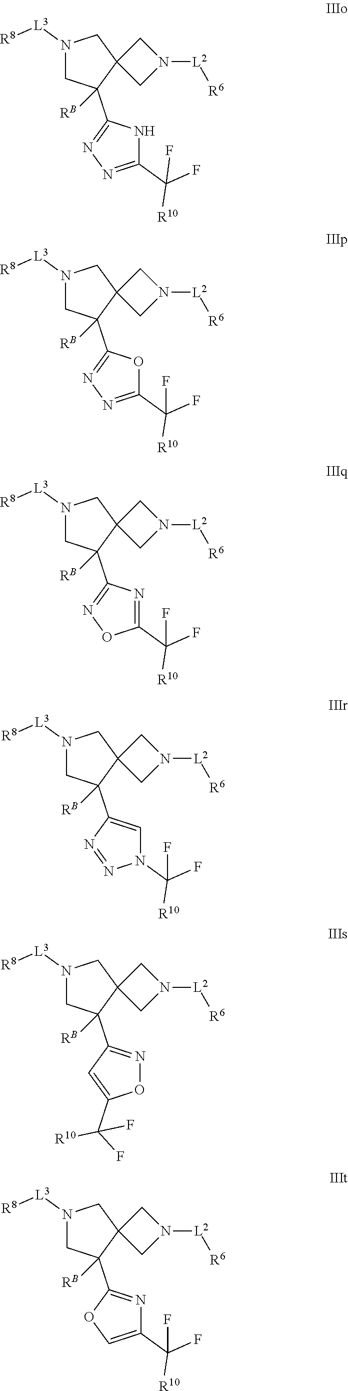 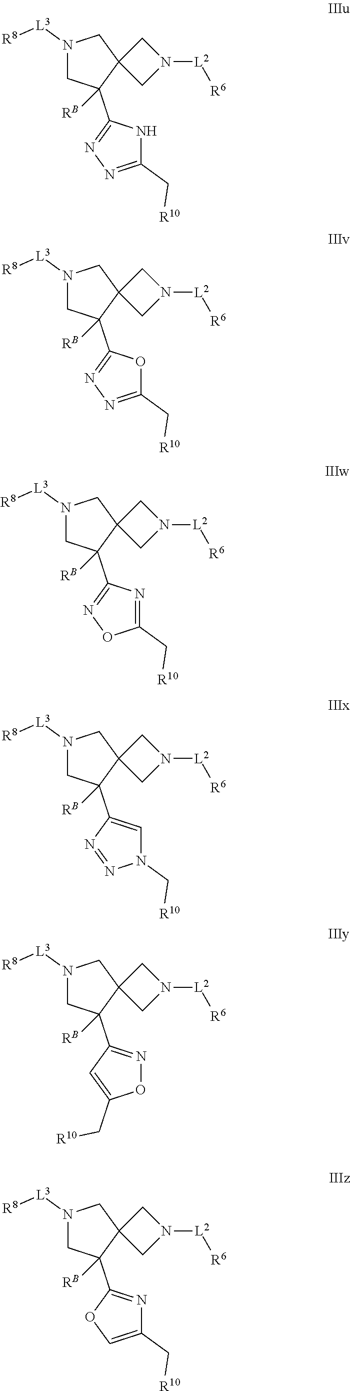
or a pharmaceutically acceptable salt thereof.

30. The compound of claim 1, wherein the compound of Formula I is a compound of Formula IVa: 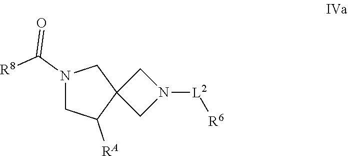
or a pharmaceutically acceptable salt thereof.

31. (canceled)

32. The compound of claim 1, wherein the compound of Formula I is a compound of Formula IVc: 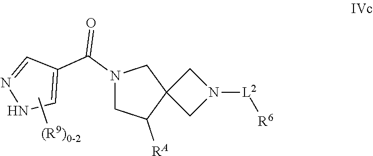
or a pharmaceutically acceptable salt thereof.

33. The compound of claim 1, wherein the compound of Formula I is a compound of Formula Va: 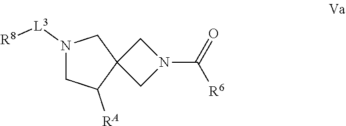
or a pharmaceutically acceptable salt thereof.

34. The compound of claim 1, wherein the compound of Formula I is a compound of Formula Vb: 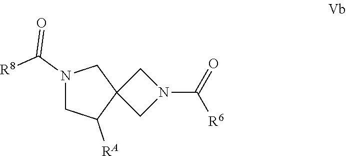
or a pharmaceutically acceptable salt thereof.

35. The compound of claim 1, wherein the compound of Formula I is a compound of Formula VIa, VIb VIc, VId, VIe, VIf, VIg, VIh, VIi, VIj, VIk, VII, VIm, VIn, VIo, VIp, VIq, VIr, VIs, VIt, VIu, VIv, VIw, VIx, VIy, VIz, VIaa, VIbb, VIcc, or VIdd: 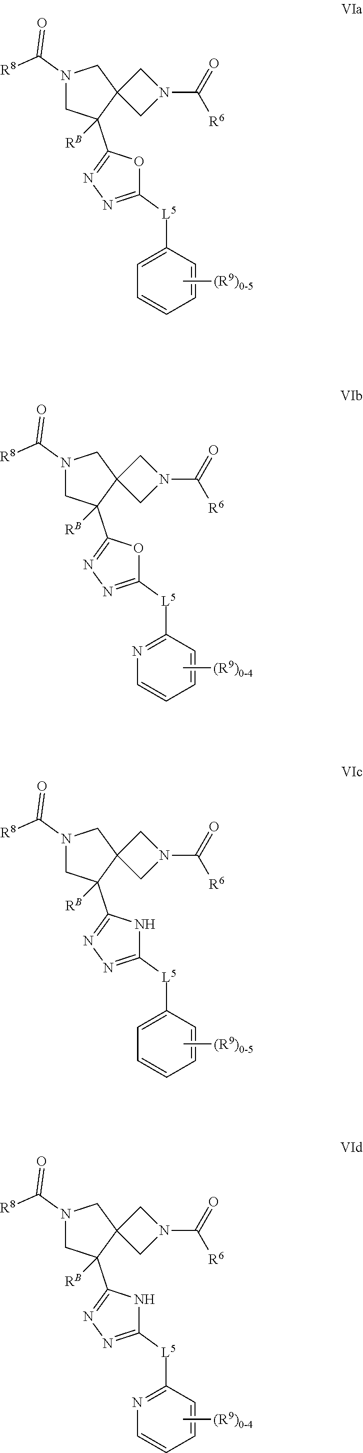 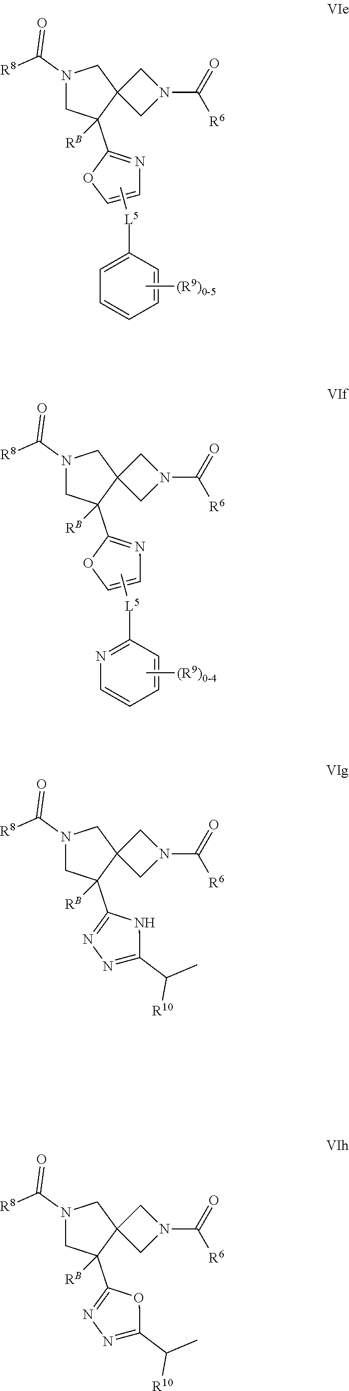 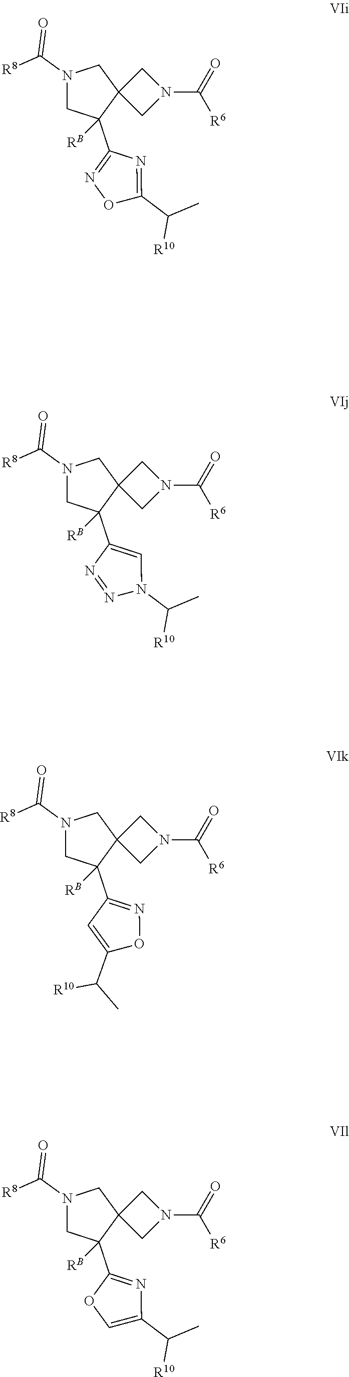 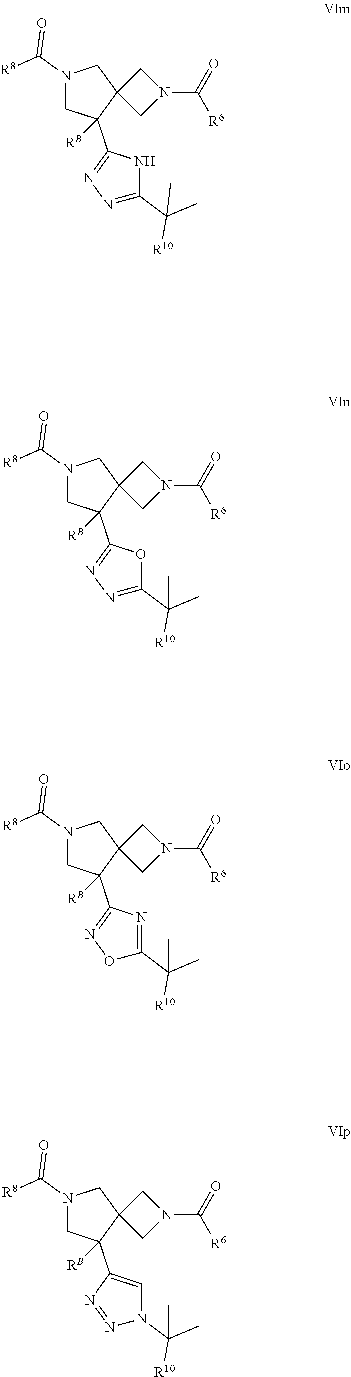 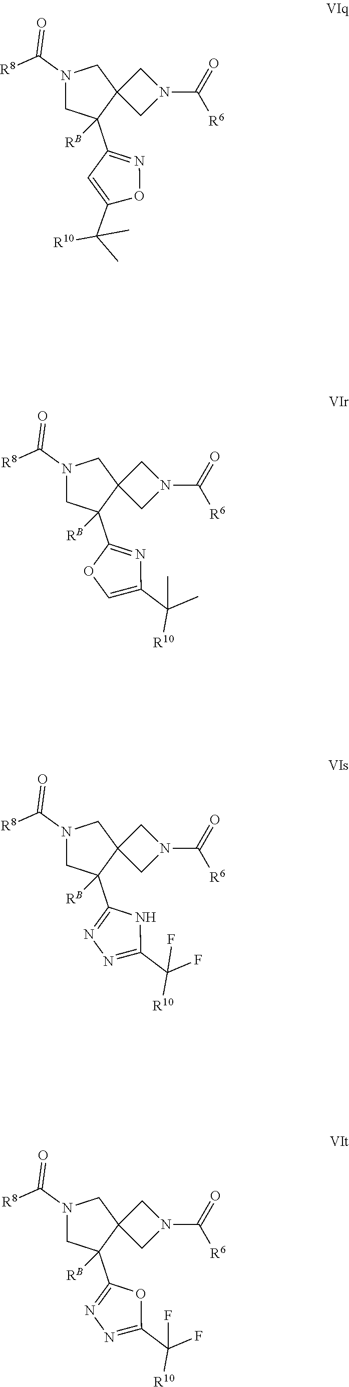 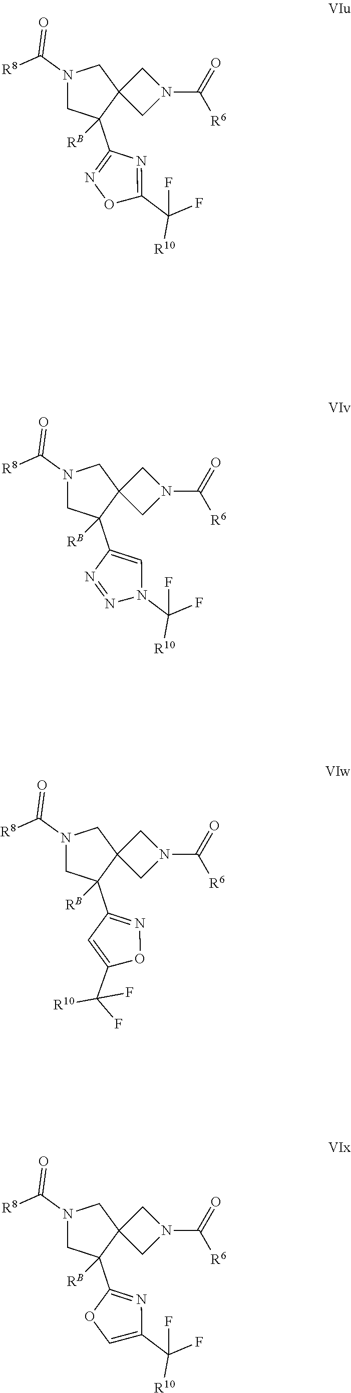 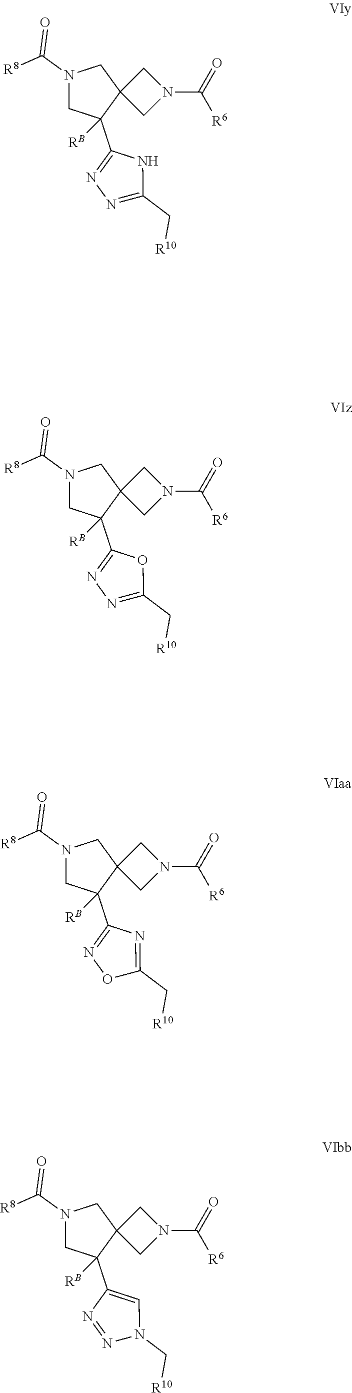 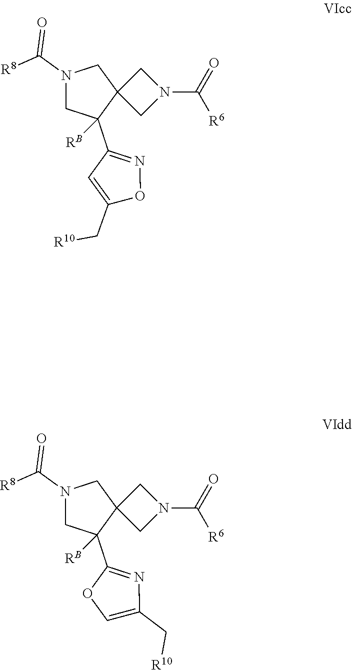
or a pharmaceutically acceptable salt thereof.

36. (canceled)

37. The compound of claim 1, wherein the compound of Formula I is a compound of Formula VIIIa, VIIIb VIIIc, VIIId, VIIIe, VIIIf, VIIIg, VIIIh, VIIIi, VIIIj, VIIIk, VIIII, VIIIm, VIIIn, VIIIo, VIIIp, VIIIq, VIIIr, VIIIs, VIIIt, VIIIu, VIIIv, VIIIw, VIIIx, VIIIy, VIIIz, VIIIaa, VIIIbb, VIIIcc, or VIIIdd: 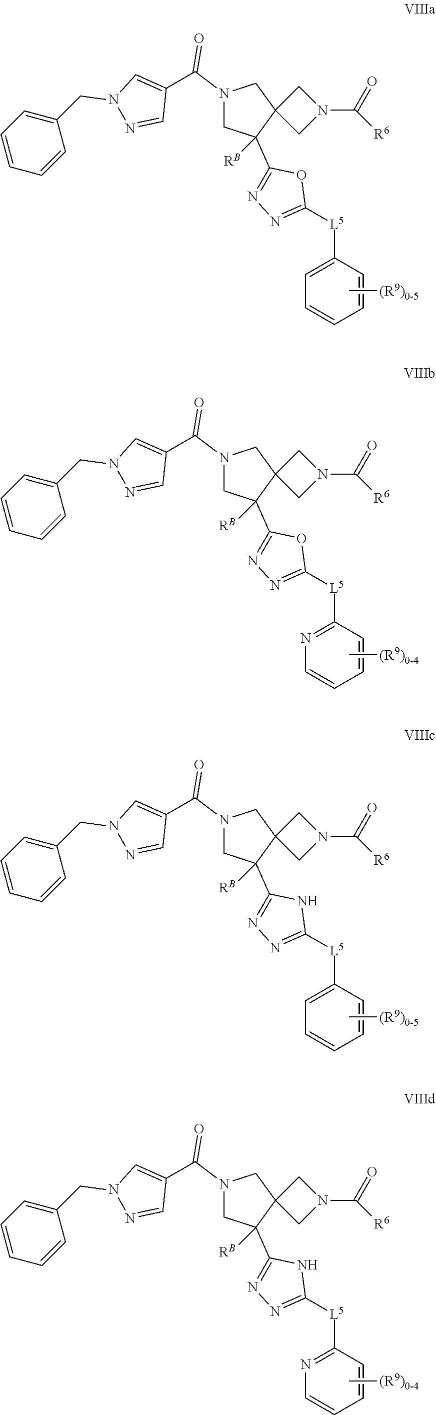 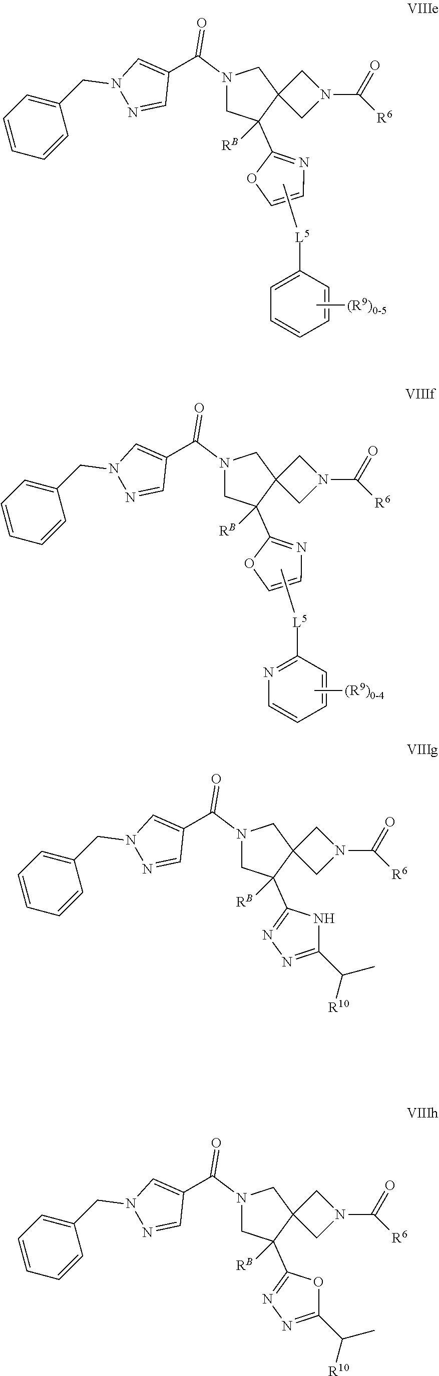 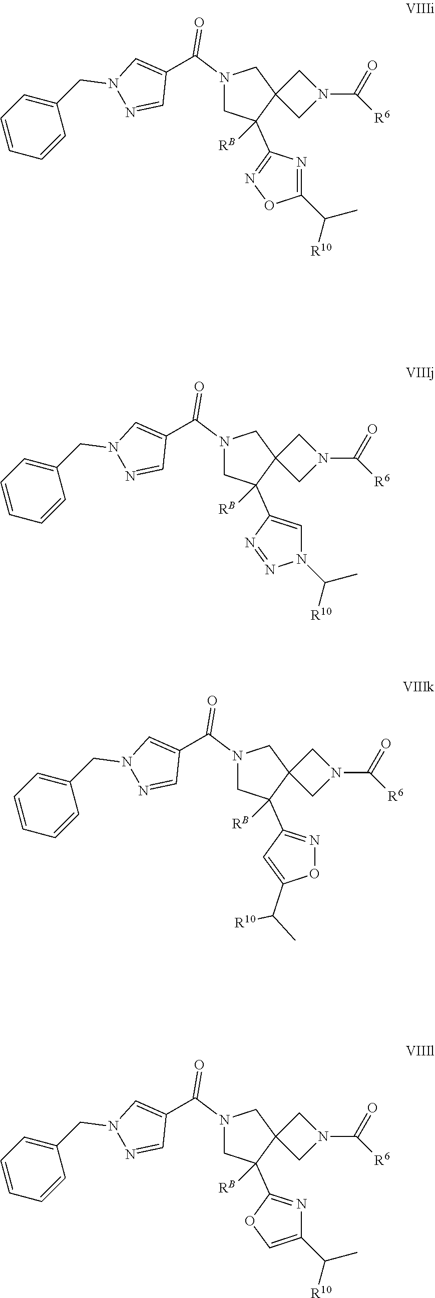 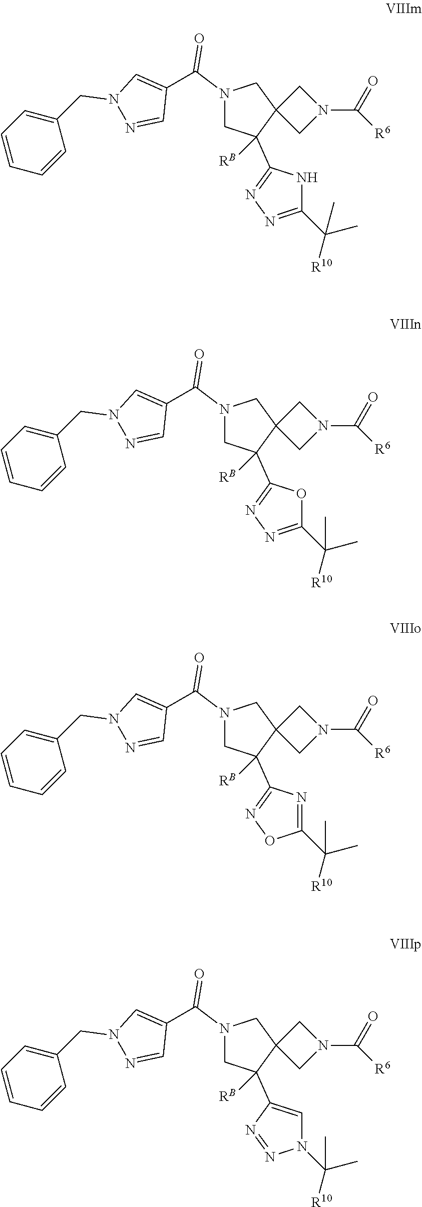 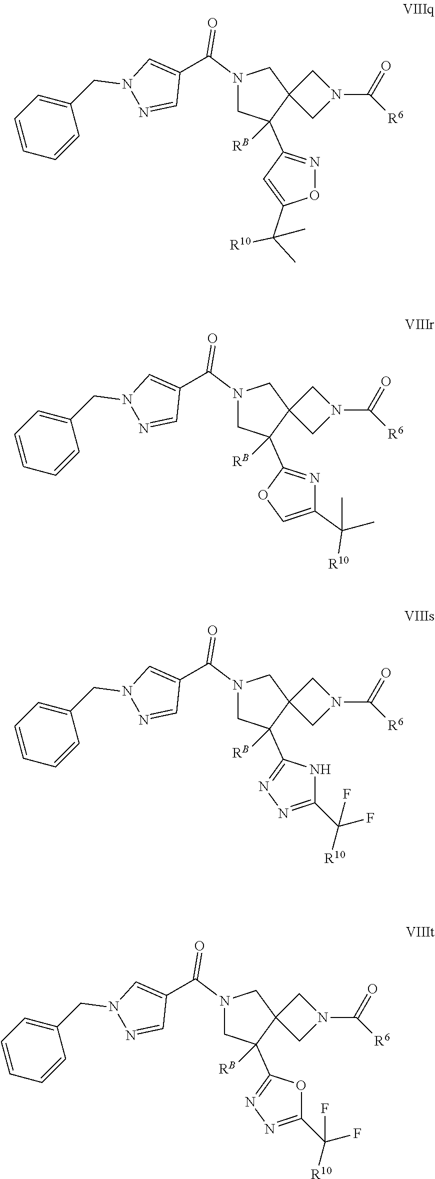 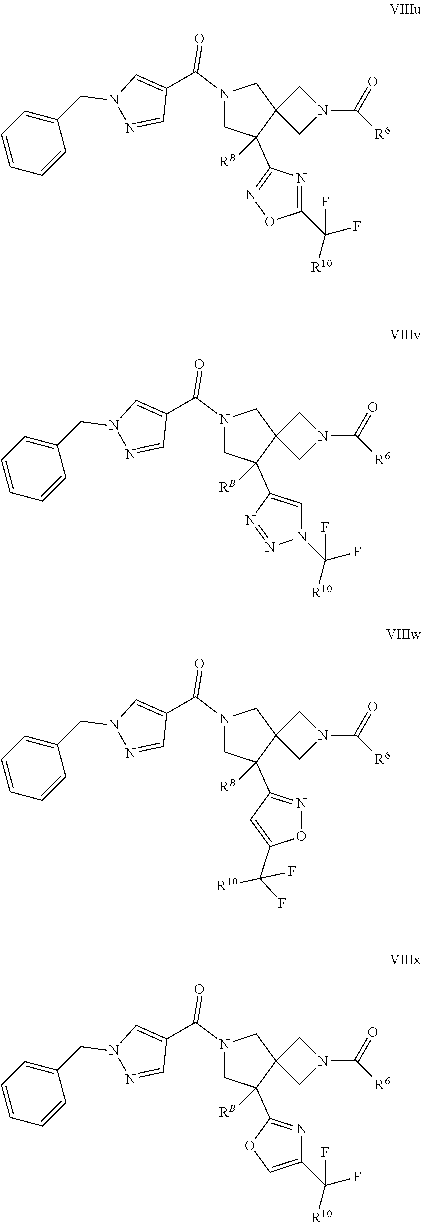 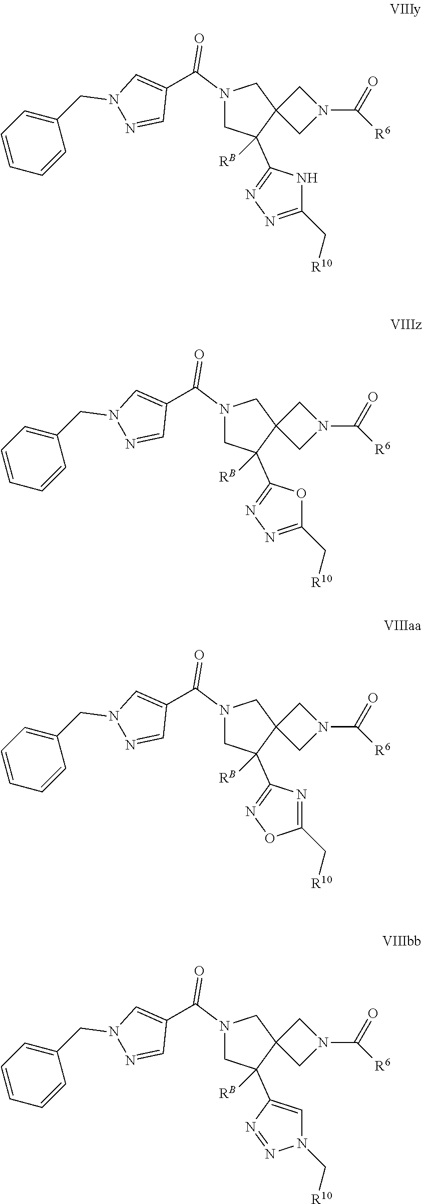 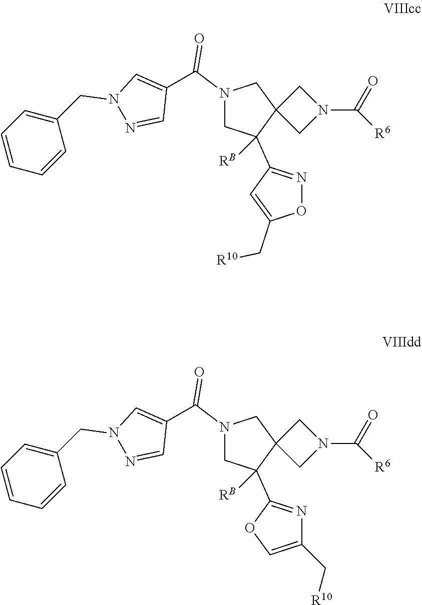
or a pharmaceutically acceptable salt thereof.

38. The compound of claim 1, wherein the compound of Formula I is a compound of Formula IXa, IXb IXc, IXd, IXe, IXf, IXg, IXh, IXi, IXj, IXk, IXl, IXm, IXn, IXo, IXp, IXq, IXr, IXs, IXt, IXu, IXv, IXw, IXx, IXy, IXz, IXaa, IXbb, IXcc, or IXdd: 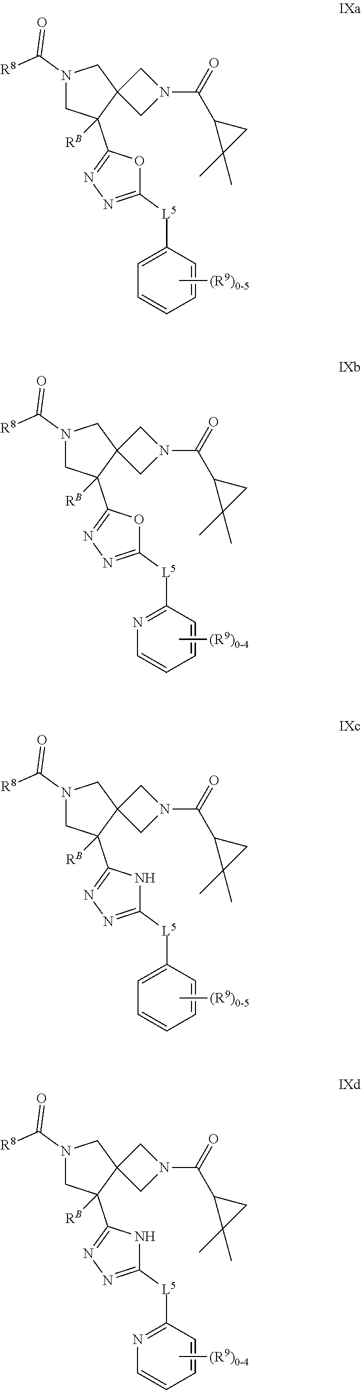 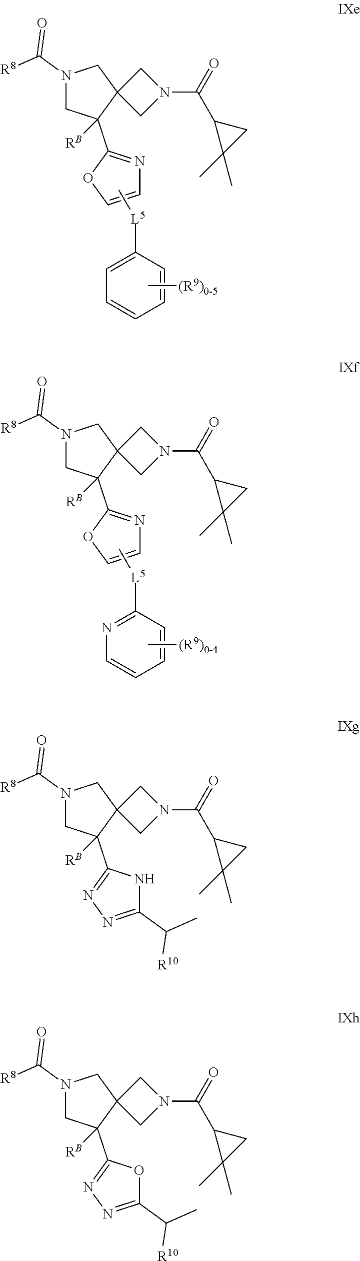 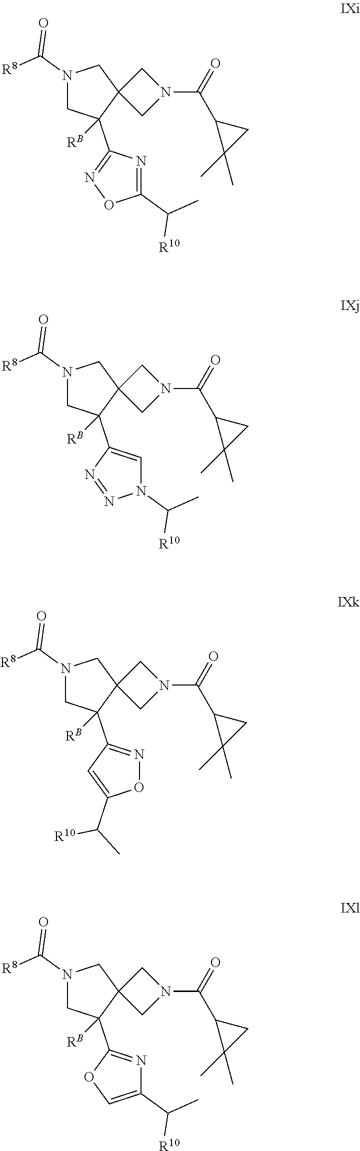 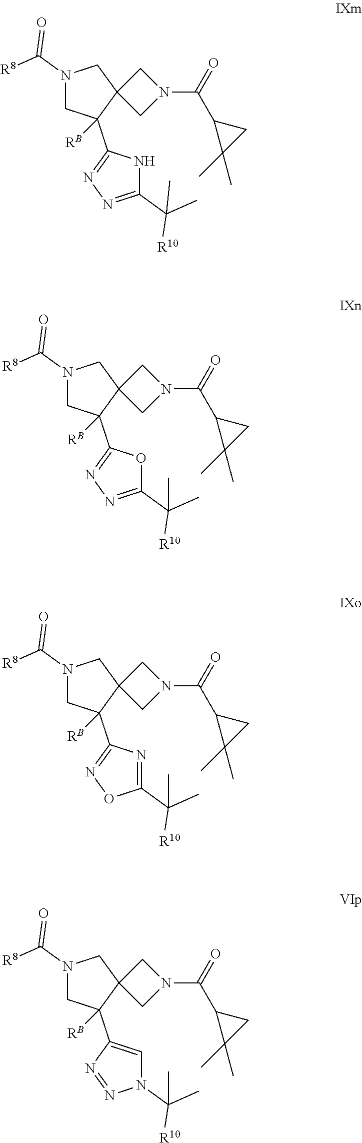 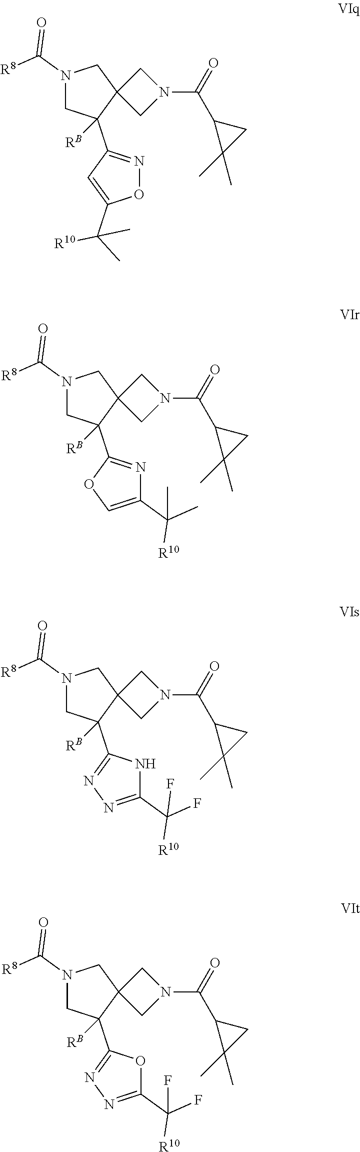 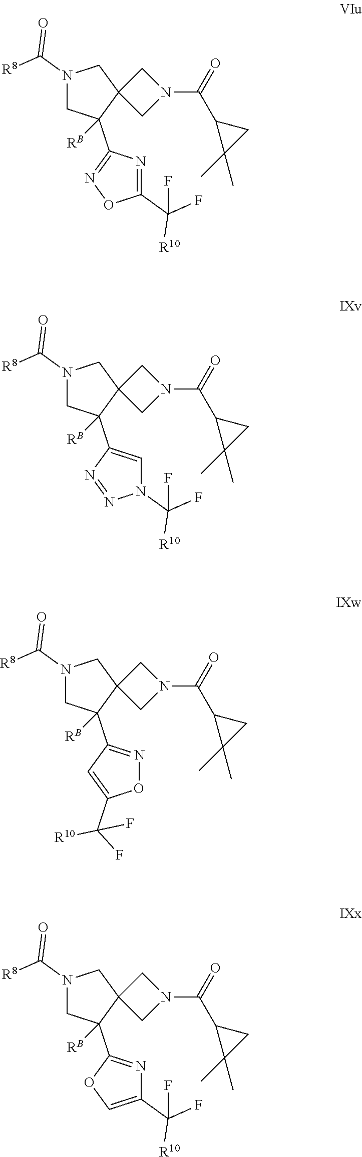 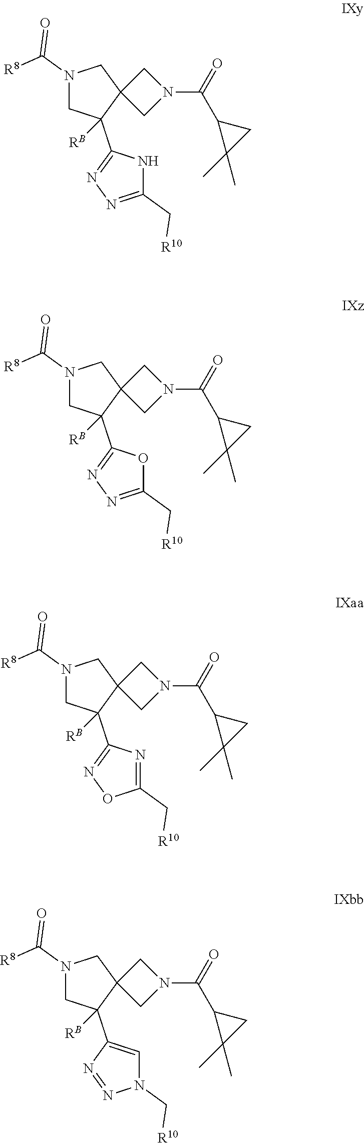 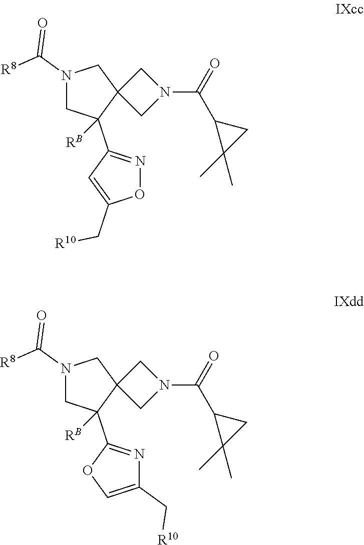
or a pharmaceutically acceptable salt thereof.

39. The compound of claim 1, wherein the compound is one of those in Table 8 or a pharmaceutically acceptable salt thereof.

40-42. (canceled)

43. A method of treating a disease or disorder associated with CDK2 activity in a patient comprising administering to the patient in need thereof a compound of claim 1, wherein the disease or disorder associated with CDK2 activity is a cancer selected from the group consisting of breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, melanoma and thyroid cancer.

44-51. (canceled)
